(19) United States
(12) Patent Application Publication
(10) Pub. No.: US 2025/0179047 A1
(43) Pub. Date: Jun. 5, 2025

(54) 5-[7-(3,4-DIHYDRO-1H-ISOQUINOLINE-2-CARBONYL)-1,2,3,4 TETRAHYDROISOQUINOLIN-6-YL]-1H-PYRROLE-3-CARBOXAMIDE DERIVATIVES, PHARMACEUTICAL COMPOSITIONS CONTAINING THEM AND THEIR USES AS PRO-APOPTOTIC AGENTS

(71) Applicants: LES LABORATOIRES SERVIER, SURESNES CEDEX (FR); VERNALIS (R&D) LTD, Cambridge (GB)

(72) Inventors: Annamária BALASSA, Budakalász (HU); Paul Andrew BROUGH, Haverhill Suffolk (GB); Audrey CLAPERON, Courbevoie (FR); Didier DEMARLES, Checy (FR); Imre FEJES, Budapest (HU); Eszter FÖRDÖS, Budapest (HU); Balázs KOMJÁTI, Budapest (HU); Daniel MADDOX, Upper Cambourne (GB); Balázs MOLNÁR, Isaszeg (HU); James Brooke MURRAY, Linton (GB); Stuart RAY, Cottenham (GB); Francesca ROCCHETTI, Vienna (AT); Emma SANDERS, London (GB); Melinda SIPOS, Budapest (HU); Zoltán SIPOS, Budapest (HU); Zoltán SZLÁVIK, Szentendre (HU); Claire Louise WALMSLEY, Ely (GB); Yikang WANG, Saffron Walden (GB)

(21) Appl. No.: 18/840,537

(22) PCT Filed: Feb. 23, 2023
(86) PCT No.: PCT/EP2023/054501

§ 371 Date: Aug. 22, 2024

(30) Foreign Application Priority Data
Feb. 24, 2022 (EP) 22305210.1
Apr. 27, 2022 (EP) 22305628.4

(51) Int. Cl. C07D 401/14; A61K 31/4725; A61K 31/496; A61K 31/5377; A61K 31/5383; A61K 31/5386; A61K 31/541; A61K 31/55; A61K 31/551; A61K 45/06; A61P 35/00; C07D 405/14; C07D 413/14; C07D 417/14; C07D 471/04; C07D 487/04; C07D 495/10; C07D 498/04

(52) U.S. Cl. CPC C07D 401/14; A61K 31/4725; A61K 31/496; A61K 31/5377; A61K 31/5383; A61K 31/5386; A61K 31/541; A61K 31/55; A61K 31/551; A61K 45/06; A61P 35/00; C07D 405/14; C07D 413/14; C07D 417/14; C07D 471/04; C07D 487/04; C07D 495/10; C07D 498/04

(57) ABSTRACT

A compound of formula (I):

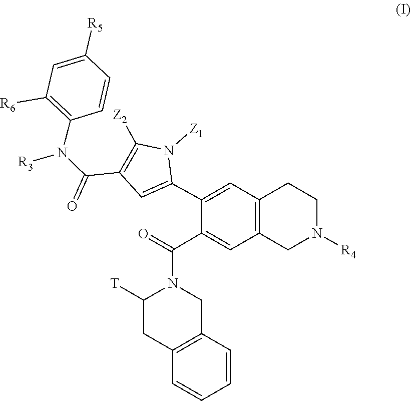

wherein R₃, R₄, R₅, R₆, Z₁, Z₂ and T are as defined in the description.

Medicinal products containing the same which are useful in treating conditions requiring anti-apoptotic inhibitors.

### FIELD OF THE INVENTION

The present invention relates to 5-[7-(3,4-dihydro-1H-isoquinoline-2-carbonyl)-1,2,3,4-tetrahydroisoquinolin-6-yl]-1H-pyrrole-3-carboxamide derivatives, to pharmaceutical compositions containing them and their uses as pro-apoptotic agents. The compounds of the present invention inhibit the activity of the Bcl-2 protein and may be of interest in the treatment of cancer, immune and autoimmune diseases.

### BACKGROUND OF THE INVENTION

Apoptosis, or programmed cell death, is a physiological process that is crucial for embryonic development and maintenance of tissue homeostasis. Apoptotic-type cell death involves morphological changes such as condensation of the nucleus, DNA fragmentation and also biochemical phenomena such as the activation of caspases which cause damage to key structural components of the cell, so inducing its disassembly and death. Regulation of the process of apoptosis is complex and involves the activation or repression of several intracellular signalling pathways (Cory S. et al., Nature Review Cancer, 2002, 2, 647-656). Deregulation of apoptosis is involved in certain pathologies. Accordingly, absence of apoptosis is one of the phenotypic signatures of cancer (Hanahan D. et al., Cell 2000, 100, 57-70). The anti-apoptotic proteins of the Bcl-2 family are associated with numerous pathologies. The involvement of proteins of the Bcl-2 family is described in numerous types of cancer, such as colon cancer, breast cancer, small-cell lung cancer, non-small-cell lung cancer, bladder cancer, ovarian cancer, prostate cancer, chronic lymphoid leukaemia, follicular lymphoma, myeloma, and prostate cancer. Overexpression of the anti-apoptotic proteins of the Bcl-2 family is involved in tumorigenesis, in resistance to chemotherapy and in the clinical prognosis of patients affected by cancer. There is, therefore, a therapeutic need for compounds that inhibit the anti-apoptotic activity of the proteins of the Bcl-2 family. Venetoclax (also known as ABT-199) is a selective Bcl-2 inhibitor that counteracts the interaction of Bcl-2 with BH3-only proteins thus inducing apoptosis. Venetoclax is approved (i) to treat adults with chronic lymphocytic leukemia (CLL) or small lymphocytic lymphoma (SLL), with or without 17p deletion, who have received at least one prior treatment and (ii) in combination with azacitidine, or decitabine, or low-dose cytarabine to treat adults with newly-diagnosed acute myeloid leukemia (AML) who are 75 years of age or older, or have other medical conditions that prevent the use of standard chemotherapy. However, since a significant number of relapses are observed in CLL, an acquired mechanism of resistance seems to appear. In other high-affinity targeted therapies, target mutations responsible for resistance have been demonstrated (Garraway & Janne, Cancer Discovery 2012, 2, 214-226). In different venetoclax-resistant derived leukemia and lymphoma cell lines, Bcl-2 mutations affecting its hydrophobic groove have been identified (Fresquet et al., Blood 2014, 123, 4111-4119; Tahir et al., BMC Cancer, 17:399). One mutation on Bcl-2 (Glycine substituted by a Valine in position 101: Gly101Val) was recently shown to be clinically relevant since identified in CLL samples from patients resistant to venetoclax. This Gly101Val (also called G101V) mutation was associated with reduced venetoclax binding to the hydrophobic groove of Bcl-2 and resistance to venetoclax (Blombery et al., Cancer Discovery 2019, 9, 342-353). Following the G101V identification, several other mutations in the BCL-2 protein were identified in patients treated with venetoclax in CLL (e.g. D103Y, D103V, D103E, F104I, F104L, D111A, A113G, R129L, V156D; Blombery et al., Blood 2020, 5; 135(10):773-777). D103E and V156D have also been detected in Relapsed refractory mantle cell lymphoma following sequential venetoclax/zanubutinib monotherapy (Thompson et al., Blood 2022, 25; 6(2):503-508). Acquired BCL-2 mutations (D111A) have also been disclosed in multiple myleoma patients treated with venetoclax (Neri et al., Blood, 134 (Supplement_1):572). F104I has also been described in a patient with follicular lymphoma (Blombery et al., Br J Hematol 2019, 186, pp. e188-e191). Based on these clinical data, there is a need to identify new therapeutic agents that can be used to treat cancer patients who carry the BCL-2 mutations following venetoclax treatment, and especially refractory and relapsed patients.

The generation of BCL-2-selective inhibitors is complicated by the high degree of similarity within the BH3-binding domains of BCL-2 and BCL-XL (Petros et al., Proc. Natl. Acad. Sci. USA 2001, 98, 3012-3017). Besides, it has been previously established that BCL-XL is also the primary survival factor in platelets (Zhang et al., Cell Death Diff 2007, May; 14(5):943-51; Mason et al., Cell 2007, 23; 128(6):1173-86). Genetic ablation, hypomorphic mutation, or pharmacologic inhibition of BCL-XL results in reduced platelet half-life and dose-dependent thrombocytopenia in vivo. Using a dual BCL-2/BCL-XL inhibitor (ABT263, navitoclax), early signs of clinical antitumor activity have been observed in lymphoid malignancies thought to be dependent on BCL-2 for survival (Wilson et al., Lancet Oncol 2010, December; 11(12):1149-59). As predicted by preclinical data, inhibition of BCL-XL by navitoclax induces a rapid, concentration-dependent decrease in the number of circulating platelets. This mechanism-based thrombocytopenia is the dose-limiting toxicity of single-agent navitoclax treatment in patients and limits the ability to drive drug concentrations into a highly efficacious range. Consequently, there is also a need to minimize the BCL-XL affinity in order to design drugs exhibiting an advantageous effectiveness-tolerability profile.

Last, drug interactions due to drug metabolism inhibition are frequent since CYP450-mediated metabolism is the major route of elimination for a large number of drugs (Deodhar et al., Pharmaceutics 2020, 4; 12(9):846). Among CYP450, CYP3A4 is the most abundant in the human liver (˜40%) and metabolizes more than 50% of clinically used drugs. Thus, drugs inhibiting CYP3A4 may cause severe drug toxicity through the enhanced exposure to co-administered drugs (Zhou et al., Ther Clin Risk Manag 2005, March; 1(1):3-13). This issue needs also to be addressed during the drug discovery process to mitigate the CYP3A4 drug-drug interaction risk.

Here, we claim the discovery of next generation BCL-2 inhibitors that are selective for BCL-2 protein with limited on-target affinity for BCL-XL and platelets loss, highly potent on BCL-2 wild type protein and showing good selectivity profile on the different BCL-2 mutations appearing in clinic following venetoclax treatment. The identified compounds also showed limited inhibition of CYP3A4.

### SUMMARY OF THE INVENTION

The present invention provides potent selective Bcl-2 inhibitors of formula (I) as defined below. We have shown that these compounds have a strong binding affinity on Bcl-2 wild-type receptor as well as on a panel of Bcl-2 mutants. The compounds of the invention demonstrated an overall better on-target affinity for the Bcl-2 proteins ac compared to Bcl-xL protein. We have also shown that the compounds of formula (I) have a cytotoxic profile in cell lines overexpressing Bcl-2 wild-type or Bcl-2 mutants. Furthermore, the compounds of formula (I) are able to induce apoptosis of cancer cells in vivo, triggering tumor regression in mice. Last, we have observed that the platelet loss in animals highly exposed to exemplary compounds is limited. Moreover, exemplary compounds also exhibited a limited inhibitory potential towards CYP3A4. Based on their pro-apoptotic properties, the compounds of the invention could be of interest for the treatment of pathologies involving a deregulation in apoptosis, such as, for example, cancer, auto-immune diseases and diseases of the immune system.

In a first aspect, the present invention provides compounds of formula (I):

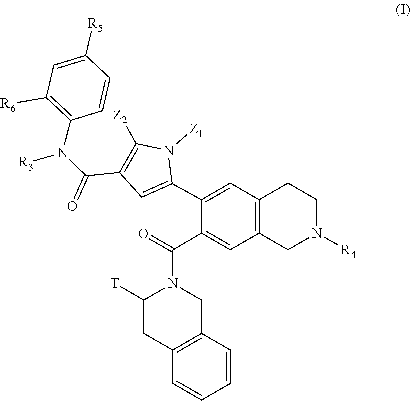

wherein:
Z₁ and Z₂ represent both a methyl group or they form together with the atoms carrying them a fused piperidine group,
T represents a hydrogen atom, a linear or branched (C₁-C₆)alkyl group optionally substituted by one to three halogen atoms, a (C₁-C₄)alkylene-NR₁R₂ group, a (C₁-C₄)alkylene-ORᵢ group,
R₁ and R₂ independently of one another represent a hydrogen atom or a linear or branched (C₁-C₆)alkyl group,
or R₁ and R₂ form with the nitrogen atom carrying them a heterocycloalkyl group, which heterocycloalkyl is optionally substituted by one to three groups selected from: (C₁-C₆)alkyl group and halogen atom,
R₃ represents a group selected from:

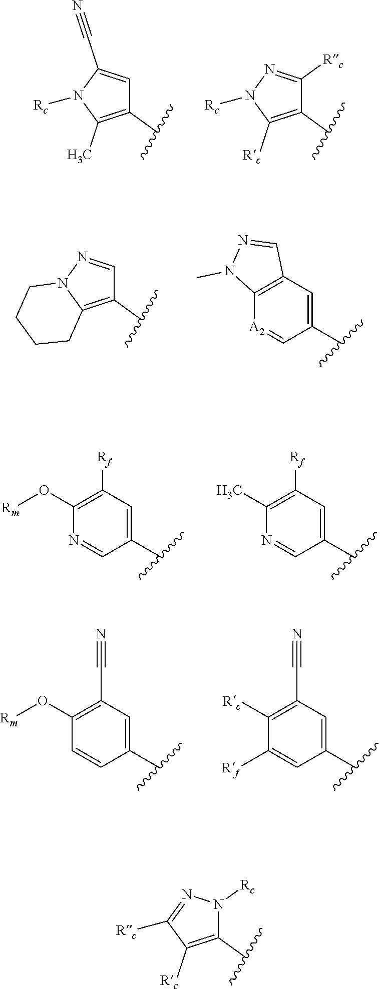

R₄ represents a group selected from:

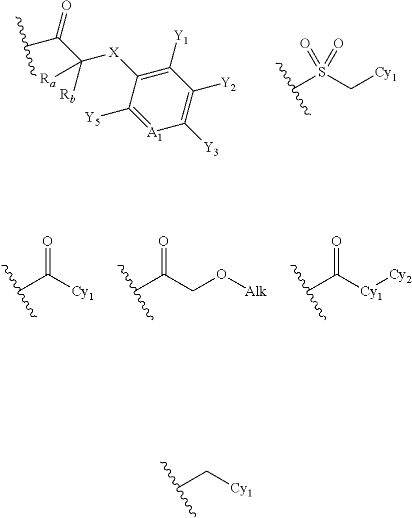

R₅ represents a hydrogen atom, a halogen atom or a hydroxy group,
R₆ represents a hydrogen, a linear or branched (C₁-C₆)alkyl group, or a halogen atom,
Alk represents a linear or branched (C₁-C₆)alkyl group,
A₁ represents a C—Y₄ or a nitrogen atom,
A₂ represents a C—H or a nitrogen atom,
Cy₁ represents a phenyl, a heteroaryl, a cycloalkyl or a heterocycloalkyl group, wherein the phenyl, the heteroaryl, the cycloalkyl and the heterocycloalkyl groups are optionally substituted by one to three groups selected from: linear or branched (C₁-C₆)alkyl group optionally substituted by 1 to 3 halogen atoms, hydroxy group, cycloalkyl group, and halogen atom and the heterocycloalkyl group is optionally further substituted by an oxo group,
Cy₂ represent a phenyl or a heteroaryl group, wherein the phenyl and the heteroaryl groups are optionally substituted by one to three groups selected from: linear or branched (C₁-C₆)alkyl group optionally substituted by 1 to 3 halogen atoms, hydroxy group, and halogen atom,
X represents a bond, —O—, —S— or NRₖ,
Y₁ and Y₅ independently of one another represent a group selected from: hydrogen atom, halogen atom, cyano, linear or branched (C₁-C₆)alkyl group, and linear or branched (C₁-C₆)alkoxy group,
Y₂ and Y₄ independently of one another represent a group selected from: hydrogen atom, halogen atom, linear or branched (C₁-C₆)alkyl group, linear or branched (C₁-C₆)alkoxy group, and heterocycloalkyl group optionally substituted by a linear or branched (C₁-C₆)alkyl group,
Y₃ represents a group selected from: hydrogen atom, halogen atom, linear or branched (C₁-C₆)alkyl, linear or branched (C₁-C₆)alkynyl, —(C₁-C₄)alkylene-OR₁, linear or branched (C₁-C₆)alkoxy group, —O-phenyl, —S-phenyl, —O—(C₁-C₄)alkylene-Cy₃, —O—(C₁-C₄)alkylene-Cy₄, —O-Cy₃, —O—(C₁-C₄)alkylene-NR_{g}Rh, —(C₁-C₄)alkylene-Cy₃, —(C₁-C₄)alkylene-Cy₄, Cy₃, Cy₄, and:

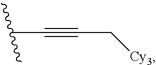

wherein the alkylene moiety of the preceding groups may be linear or branched,
Cy₃ represents a heterocycloalkyl optionally substituted by one to three groups selected from: linear or branched (C₁-C₆)alkyl group optionally substituted by 1 to 3 halogen atoms, hydroxy group, cycloalkyl group, heterocycloalkyl group, and halogen atom,
Cy₄ represents a cycloalkyl optionally substituted by one to three groups selected from: linear or branched (C₁-C₆)alkyl group optionally substituted by 1 to 3 halogen atoms, hydroxy group, cycloalkyl group, heterocycloalkyl group, and halogen atom
Rₐ and R_{b} independently of one another represent a hydrogen atom or a halogen atom,
R_{c} represents a group selected from: hydrogen, linear or branched (C₁-C₆)alkyl group optionally substituted by 1 to 3 halogen atoms, (C₁-C₆)alkylene-NR_{d}Rₑ, (C₁-C₆)alkylene-ORⱼ, cycloalkyl, heterocycloalkyl, and (C₁-C₆)alkylene-heterocycloalkyl group,
R′_{c} and R″_{c} independently of one another represent a hydrogen atom or a linear or branched (C₁-C₆)alkyl (preferably a methyl),
R_{d} and Rₑ independently of one another represent a hydrogen atom, a linear or branched (C₁-C₆)alkyl group, a cycloalkyl group or a heterocycloalkyl group,
R_{f} represents a hydrogen atom, a halogen atom or a cyano group,
R′_{f} represents a hydrogen atom or a halogen atom,
R_{g} and Rₕ independently of one another represent a hydrogen atom, a linear or branched (C₁-C₆)alkyl group optionally substituted by one to three halogen atoms, a cycloalkyl group, a heterocycloalkyl group, or a—(C₁-C₆)alkylene-heterocycloalkyl,
Rᵢ, Rⱼ, and Rₖ independently of one another represent a hydrogen atom, a linear or branched (C₁-C₆)alkyl group, or a—(C₁-C₆)alkylene-cycloalkyl group,
R₁ represents a hydrogen atom, a linear or branched (C₁-C₆)alkyl group or a linear or branched (C₁-C₆)alkylene-heterocycloalkyl group,
Rₘ represents a hydrogen or a linear or branched (C₁-C₆)alkyl group,
   its enantiomers and diastereoisomers, and addition salts thereof with a pharmaceutically acceptable acid or base.

In another aspect, the invention provides compounds of formula (I) as described herein, for use in the treatment of cancer, autoimmune diseases and the disease of immune system.

In a further aspect, the invention provides a pharmaceutical composition comprising the compounds of Formula (I) as described herein, and at least one pharmaceutically acceptable excipient.

### Definitions

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, etc.

Among the pharmaceutically acceptable bases there may be mentioned, without implying any limitation, sodium hydroxide, potassium hydroxide, triethylamine, tert-butylamine, etc.

“heteroaryl” means any monocyclic or fused bicyclic group composed of from 5 to 12 ring members, having at least one aromatic moiety and containing from 1 to 4 hetero atoms selected from oxygen, sulphur and nitrogen. Among the heteroaryl groups, there may be mentioned, without implying any limitation, furyl, thienyl, thiazolyl, isoxazolyl, pyrrolyl, pyrazolyl, pyridinyl (also known as pyridyl), pyrimidinyl, indolyl, dihydroindolyl, indazolyl, tetrahydroindazolyl, benzofuranyl, dihydrobenzofuranyl, benzimidazolyl, benzopyranyl, benzodioxolyl, quinolinyl, dihydroquinolinyl, tetrahydroquinolinyl, tetrahydroquinazolinyl, pyrrolopyridinyl, thienopyrimidinyl, furopyridinyl, benzothiazolyl, isothiazolyl, oxazolyl, imidazolyl, pyrazinyl, pyridazinyl, dihydroisoindolyl, benzothienyl, tetrahydrobenzothienyl, isoquinolinyl, tetrahydroisoquinolinyl, quinoxalinyl, dihydroquinoxalinyl, 4H,5H,6H,7H-pyrazolo[1,5-a]pyridinyl, pyrazolo[3,4-b]pyridinyl, etc.
“cycloalkyl” means any mono- or bi-cyclic non-aromatic carbocyclic group containing from 3 to 10 ring members, which may include fused, bridged or spiro ring systems. Among the cycloalkyl groups, there may be mentioned, without implying any limitation, cyclopropyl, cyclobutyl, cyclopentyl, cyclohexyl, etc.
“heterocycloalkyl” means any mono- or bi-cyclic non-aromatic carbocyclic group, composed of from 3 to 10 ring members, and containing from one to 3 hetero atoms or groups selected from oxygen, sulphur, SO, SO₂ and nitrogen, wherein the bicyclic group may be fused or spiro type. Heterocycloalkyl group may have one double bond. Among the heterocycloalkyl groups, there may be mentioned, without implying any limitation, azetidinyl, tetrahydrofuranyl, tetrahydropyridinyl, piperidinyl, piperazinyl, morpholinyl, thiomorpholinyl, pyrrolidinyl, oxanyl (also called tetrahydropyranyl), 1,4-dioxanyl, oxetanyl, azepanyl, 1,4-diazepanyl, (9aS)-hexahydro-1H-piperazino[2,1-c]morpholinyl (also called (9aS)-octahydropyrazino[2,1-c][1,4]oxazinyl), 1,1-dioxo-1)⁶-thia-6-azaspiro[3.3]heptanyl, (8aS)-hexahydropyrrolo[1,2-a]piperazinyl, 6-oxa-9-azaspiro[4.5]decanyl, 4-oxa-7-azaspiro[2.5]octanyl, 6-azaspiro[2.5]octanyl, 5-azaspiro[2.3]hexanyl, 2-azaspiro[3.3]heptanyl, 5-azaspiro[2.4]heptanyl, 2,6-diazaspiro[3.3]heptanyl, (1RS,5SR)-3-azabicyclo[3.1.0]hexanyl (also called cis-3-azabicyclo[3.1.0]hexanyl), etc.

“(C₁-C₆)alkylene” means a divalent linear or branched, saturated hydrocarbon radical having from 1 to 6 carbon atoms. Among the alkylene radicals, there may be mentioned, without implying any limitation, —CH₂—, —(CH₂)₂—, —(CH₂)₃—, —(CH₂)₄—, —CH(CH₃)—, —CH₂—CH(CH₃)—, —CH(CH₃)—CH₂—, —CH₂—CH(CH₃)—CH₂—, —CH₂—CH(CH₂—CH₃)—CH₂—, —CH₂—CH[CH(CH₃)₂]—CH₂—, —CH₂—C(CH₃)₂—CH₂—, —CH₂—CH(CH₃)—CH(CH₃)—, etc.

Among the pharmaceutical compositions according to the invention there may be mentioned more especially those that are suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels, and drinkable or injectable ampoules. The pharmaceutical compositions according to the invention comprise one or more excipients or carriers selected from diluents (such as lactose, dextrose, sucrose, mannitol, sorbitol, cellulose, glycerol . . . ), lubricants (such as silica, talc, stearic acid and its magnesium and calcium salts, polyethylene glycol . . . ), binders (such as magnesium aluminum silicate, starch, gelatin, tragacanth, methylcellulose, sodium carboxymethylcellulose and polyvinylpyrrolidone . . . ), disintegration agents (such as agar, alginic acid and its sodium salt, effervescent mixtures . . . ), stabilizers, preservatives, absorbents, colorants, sweeteners, flavorings, etc. The administration route is preferably the oral route or the intravenous route, and the corresponding pharmaceutical compositions may allow the instantaneous or delayed release of the active ingredients.

Among the combinations of a compound of formula (I) with an anticancer agent according to the invention, there may be mentioned more especially those that are suitable for a simultaneous administration or a sequential administration. The combinations according to the invention comprise a compound of formula (I) combined to anti-cancer agents selected from genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies. The compounds of the combination may moreover be administered in the form of two separate pharmaceutical compositions, each containing one of the active ingredients, or in the form of a single pharmaceutical composition, in which the active ingredients are in admixture.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both.

Among the cancer treatments envisaged there may be mentioned, without implying any limitation, the treatment of haematological malignancies and solid tumors. Haematological malignancies include myeloma, especially multiple myeloma, lymphoma, especially Non-Hodgkin Lymphoma (NHL) and Diffuse Large B-cell Lymphoma (DLBCL), and leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML). Solid tumors include the bladder, brain, breast, uterus, osophagus and liver cancers, colorectal cancer, renal cancer, melanoma, ovarian cancer, prostate cancer, pancreatic cancer and lung cancer, especially non-small-cell lung cancer and small-cell lung cancer.

Among the treatments of autoimmune diseases envisaged there may be mentioned, without implying any limitation, the treatment of rheumatoid arthritis (RA) and systemic lupus erythematosus (SLE).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts. A suitable daily dose of a compound of the invention will depend upon the factors described above and may range from 0.01 mg to 2.5 g per day in one or more administration(s).

### DETAILED DESCRIPTION

Advantageously, the compound of the invention has the formula (I-a):

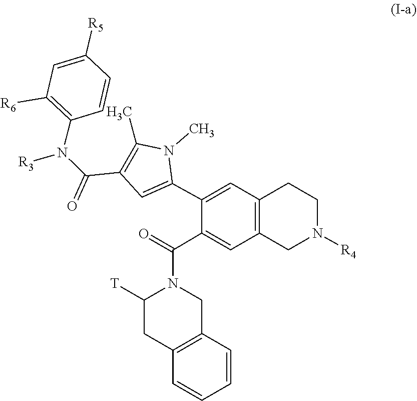

wherein R₃, R₄, R₅, R₆ and T are as defined in claim 1.

In a preferred embodiment, R₃ represents the following group:

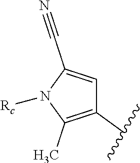

and R_{c} represents a group selected from: hydrogen, linear or branched (C₁-C₆)alkyl group optionally substituted by 1 to 3 halogen atoms, (C₁-C₆)alkylene-NR_{d}Rₑ, (C₁-C₆)alkylene-ORⱼ, cycloalkyl, heterocycloalkyl, and (C₁-C₆)alkylene-heterocycloalkyl group. Preferably, R, represents a group selected from: methyl, 2-hydroxyethyl, 2-[cyclopropyl(methyl)amino]ethyl, ethyl, isopropyl, 2-methoxyethyl, cyclopropyl, difluoromethyl, and tetrahydrofuran-3-ylmethyl. Even more preferably, R_{c} represents a methyl group.

In another embodiment, R₃ represents the following group:

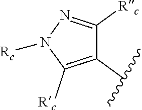

and R_{c} represents a group selected from: methyl, ethyl, oxanyl, 2-methoxyethyl, 2-methylpropyl, difluoromethyl, and 2-(dimethylamino)ethyl. Even more preferably, R_{c} represents a methyl group.

In some embodiments, R₄ represents the following group:

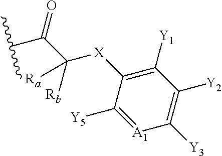

wherein Rₐ, R_{b}, X, A₁, Y₁, Y₂, Y₃, Y₅ are as defined in E1.

Described below are a number of embodiments of the invention, where for convenience E1 is identical to the first aspect of the invention hereinabove. Further enumerated embodiments (E) of the invention are described herein. It will be recognized that features specified in each embodiment may be combined with other specified features to provide further embodiments of the present invention.
E2. The compound according to E1 having the formula (I-b):

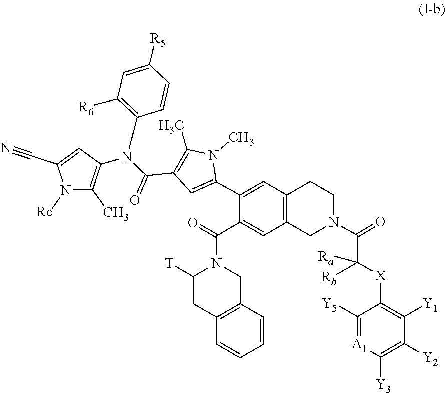

wherein R₅, R₆, Rₐ, R_{b}, R_{c}, X, A₁, Y₁, Y₂, Y₃, Y₅ and T are as defined in E1.

Advantageously, R_{c} represents a methyl group.
E3. The compound according to E1 having the formula (I-c).

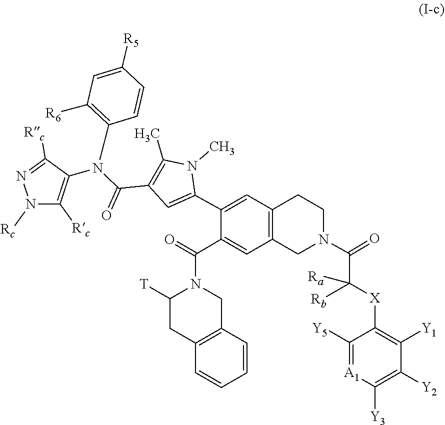

wherein R₅, R₆, Rₐ, R_{b}, R_{c}, R′_{c}, R″_{c}, X, A₁, Y₁, Y₂, Y₃, Y₅ and T are as defined in E1.
E4. The compound according to E1 having the formula (I-d):

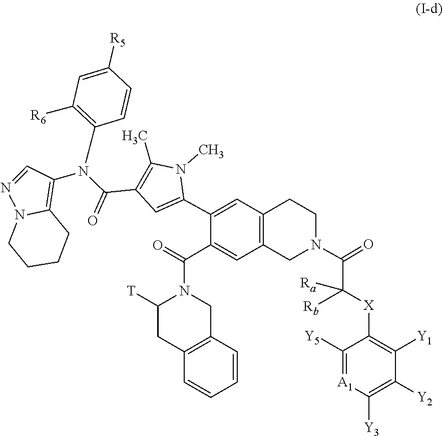

wherein R₅, R₆, Rₐ, R_{b}, X, A₁, Y₁, Y₂, Y₃, Y₅ and T are as defined in E1.
E5. The compound according to E1 having the formula (I-e).

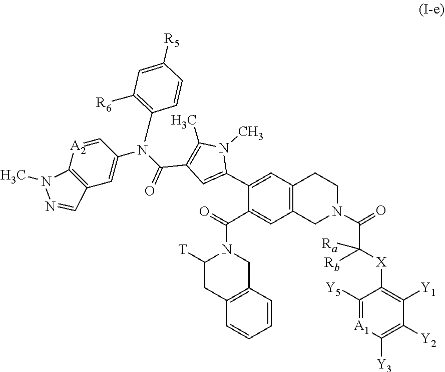

wherein R₅, R₆, Rₐ, R_{b}, X, A₁, A₂, Y₁, Y₂, Y₃, Y₅ and T are as defined in E1.
E6. The compound according to E1 having the formula (I-f):

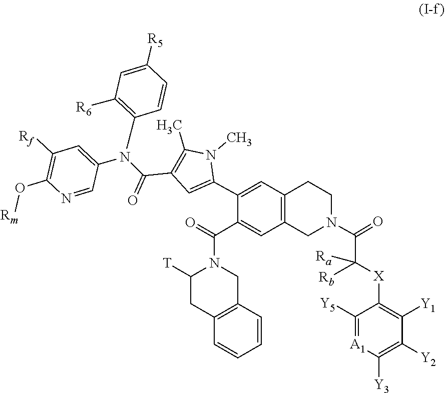

wherein R₅, R₆, Rₐ, R_{b}, R_{f}, Rₘ, X, A₁, Y₁, Y₂, Y₃, Y₅ and T are as defined in E1.

Preferably, Rₘ represents a methyl group.
E7. The compound according to E1 having the formula (I-g):

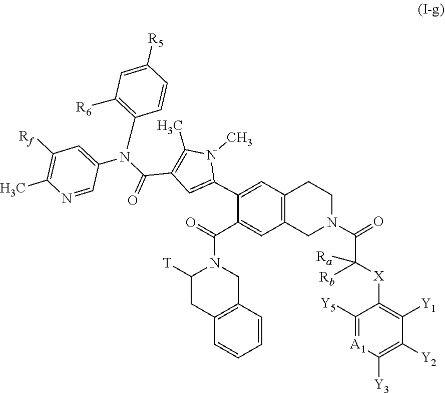

wherein R₅, R₆, Rₐ, R_{b}, R_{f}, X, A₁, Y₁, Y₂, Y₃, Y₅ and T are as defined in E1.
E8. The compound according to E1 having the formula (I-h):

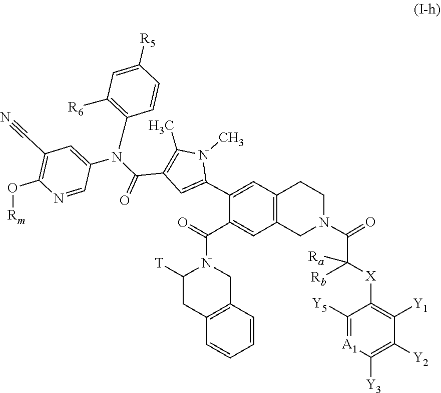

wherein R₅, R₆, Rₐ, R_{b}, Rₘ, X, A₁, Y₁, Y₂, Y₃, Y₅ and T are as defined in E1.

Preferably, Rₘ represents a methyl group.
E9. The compound according to E1 having the formula (I-i):

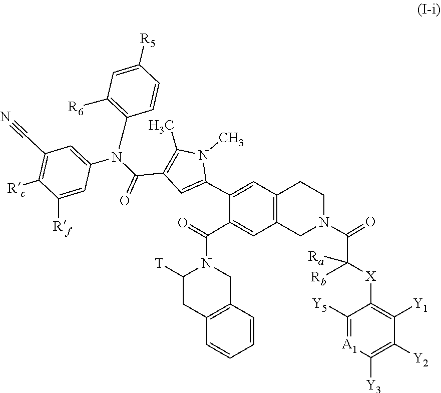

wherein R₅, R₆, Rₐ, R_{b}, R′_{c}, R′_{f}, X, A₁, Y₁, Y₂, Y₃, Y₅ and T are as defined in E1.
E10. The compound according to E1 having the formula (I-j):

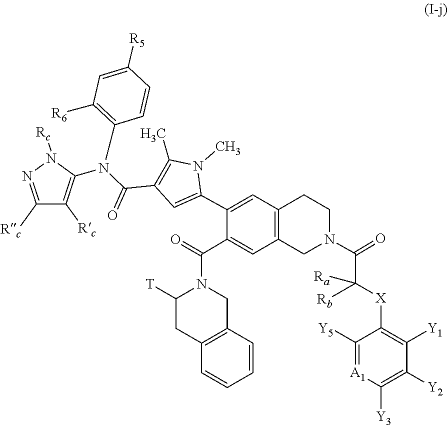

wherein R₅, R₆, Rₐ, R_{b}, R_{c}, R′_{c}, R″_{c}, X, A₁, Y₁, Y₂, Y₃, Y₅ and T are as defined in E1.
E11. The compound according to any of E2 to E10 wherein X represents a bond.
E12. The compound according to any of E2 to E10 wherein A₁ represents C—Y₄.
E13. The compound according to any of E2 to E10 wherein R % and R_{b} represent both a hydrogen atom.
E14. The compound according to any of E2 to E10 wherein R₅ represents a hydrogen atom, a hydroxy group or a fluorine atom, preferably a hydroxy group.
E15. The compound according to any of E2 to E10 wherein R₆ represents a hydrogen atom or a fluorine atom, preferably a hydrogen atom.
E16. The compound according to any of E2 to E10 wherein R₅ represents a hydroxy group and R₆ represents a hydrogen atom.
E17. The compound according to any of E2 to E10 wherein A₁ represents C—H and Y₂ represents a hydrogen atom.
E18. The compound according to any of E2 to E10 wherein Y₁ and Y₅ represent both a hydrogen atom,
   or: Y₁ and Y₅ represent a fluoro atom and a hydrogen atom, respectively.
E19. The compound according to any of E2 to E10 wherein Y₃ represents a—O—(C₁-C₄)alkylene-Cy₃ group. Preferably, the (C₁-C₄)alkylene group is ethylene.
E20. The compound according to any of E2 to E10 wherein Y₃ represents a—O—(C₁-C₄)alkylene-NR_{g}Rₕ. Preferably, the (C₁-C₄)alkylene group is ethylene.

Even more preferably, Y₃ represents a group selected from: 2-(morpholin-4-yl)ethoxy, 2-[4-(2,2-difluoroethyl)piperazin-1-yl]ethoxy, 2-(3-fluoroazetidin-1-yl)ethoxy, 2-(3,3-difluoropyrrolidin-1-yl)ethoxy, 2-(oxan-4-yl)ethoxy, 2-(4-fluoropiperidin-1-yl)ethoxy, 2-(thiomorpholin-4-yl)ethoxy, 2-(2-methylmorpholin-4-yl)ethoxy, 2-{6-oxa-9-azaspiro[4.5]decan-9-yl}ethoxy, 2-{4-oxa-7-azaspiro[2.5]octan-7-yl}ethoxy, 2,6-dimethylmorpholin-4-yl]ethoxy, 2-[cyclopropyl(methyl)amino]ethoxy, 2-{methyl[(oxetan-3-yl)methyl]amino}ethoxy, 2-[methyl(oxetan-3-yl)amino]ethoxy, 2-(4-fluoropiperidin-1-yl)ethoxy, 2-[(2-fluoroethyl)(methyl)amino]ethoxy, 2-[4-(2-fluoroethyl)piperazin-1-yl]ethoxy, 2-(4-methylpiperazin-1-yl)ethoxy, 2-(2,2-dimethylmorpholin-4-yl)ethoxy, 2-(morpholin-4-yl)propoxy, 2-(4,4-difluoropiperidin-1-yl)ethyl, [2-methyl-1-(morpholin-4-yl)propan-2-yl]oxy, 2-(3,3-dimethylmorpholin-4-yl)ethoxy, and [(oxan-4-yl)methoxy]methyl.

In some embodiments, Y₃ represents a group selected from: 2-(morpholin-4-yl)ethoxy, 2-(oxan-4-yl)ethoxy, 2-(4-hydroxypiperidin-1-yl)ethoxy, 2-(4-cyclopropylpiperazin-1-yl)ethoxy, 2-[4-(2,2,2-trifluoroethyl)piperazin-1-yl]ethoxy, 2-[(9aS)-octahydropyrazino[2,1-c][1,4]oxazin-8-yl]ethoxy, 2-{2-[4-(2-{1,1-dioxo-1λ⁶-thia-6-azaspiro[3.3]heptan-6-yl}ethoxy, 2-[2,6-dimethylmorpholin-4-yl]ethoxy, 2-[4-(2,2-difluoroethyl)piperazin-1-yl]ethoxy, 2-(3-fluoroazetidin-1-yl)ethoxy, 2-(3,3-difluoropyrrolidin-1-yl)ethoxy, 2-(4-fluoropiperidin-1-yl)ethoxy, 2-(thiomorpholin-4-yl)ethoxy, 2-(2-methylmorpholin-4-yl)ethoxy, 2-{6-oxa-9-azaspiro[4.5]decan-9-yl}ethoxy, 2-{4-oxa-7-azaspiro[2.5]octan-7-yl}ethoxy, 2-[4-(2-fluoroethyl)piperazin-1-yl]ethoxy, 2-(4-methylpiperazin-1-yl)ethoxy, 2-(2,2-dimethylmorpholin-4-yl)ethoxy, 2-(morpholin-4-yl)propoxy, [2-methyl-1-(morpholin-4-yl)propan-2-yl]oxy, 2-(3,3-dimethylmorpholin-4-yl)ethoxy, 2-(3-methylmorpholin-4-yl)ethoxy, 2-(1,4-dioxan-2-yl)ethoxy.

In some other embodiments, Y₃ represents a group selected from: 2-[cyclopropyl(methyl)amino]ethoxy, 2-[methyl(oxetan-3-yl)amino]ethoxy, 2-{methyl[(oxetan-3-yl)methyl]amino}ethoxy, 2-[(2-fluoroethyl)(methyl)amino]ethoxy, 2-(dimethylamino)ethoxy, 2-(morpholin-4-yl)ethyl, 2-(4,4-difluoropiperidin-1-yl)ethyl.

Another embodiment of the present disclosure relates to the compound of formula (I) wherein the group:

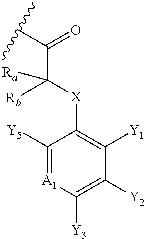

represents

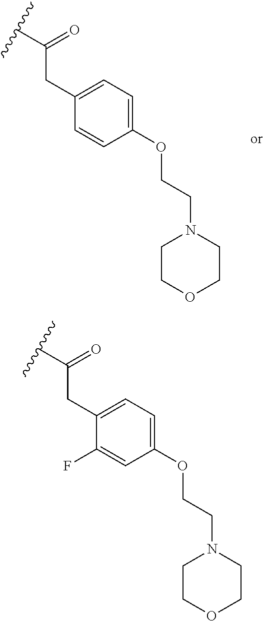 [SMILES: *C(=O)Cc1ccc(OCCN2CCOCC2)cc1 ; *C(=O)Cc1ccc(OCCN2CCOCC2)cc1F]

In some embodiments, T represents a linear or branched (C₁-C₆)alkyl group or a (C₁-C₄)alkylene-NR₁R₂ group. Advantageously, T represents a group selected from: methyl group, (piperidin-1-yl)methyl, (morpholin-4-yl)methyl, [(3R)-3-fluoropyrrolidin-1-yl]methyl, [methyl(propan-2-yl)amino]methyl, (azepan-1-yl)methyl, (pyrrolidin-1-yl)methyl, [(3S)-3-methylpiperidin-1-yl]methyl, [(3R)-3-methylpiperidin-1-yl]methyl, [(1RS,5SR)-3-azabicyclo[3.1.0]hexan-3-yl]methyl, [(2S)-2-methylpiperidin-1-yl]methyl, {6-azaspiro[2.5]octan-6-yl}methyl, (4,4-difluoropiperidin-1-yl)methyl, (4-methylpiperidin-1-yl)methyl, [ethyl(propan-2-yl)amino]methyl, [(3R)-3-methylpyrrolidin-1-yl]methyl, and [(3S)-3-methylpyrrolidin-1-yl]methyl. In an other embodiment, T represents a group selected from: methyl group, (piperidin-1-yl)methyl, (morpholin-4-yl)methyl, (piperidin-1-yl)ethyl, [(3R)-3-fluoropyrrolidin-1-yl]methyl, (4-fluoropiperidin-1-yl)methyl, [methyl(propan-2-yl)amino]methyl, (azepan-1-yl)methyl, (pyrrolidin-1-yl)methyl, [(3S)-3-methylpiperidin-1-yl]methyl, [(3R)-3-methylpiperidin-1-yl]methyl, [(1RS,5SR)-3-azabicyclo[3.1.0]hexan-3-yl]methyl, [(2S)-2-methylpiperidin-1-yl]methyl, {6-azaspiro[2.5]octan-6-yl}methyl, (4,4-difluoropiperidin-1-yl)methyl, (diethylamino)methyl, (4-methylpiperidin-1-yl)methyl, [ethyl(propan-2-yl)amino]methyl, {5-azaspiro[2.3]hexan-5-yl}methyl, (3,3-dimethylpyrrolidin-1-yl)methyl, (diisopropylamino)methyl, [ethyl(isopropyl) amino]methyl, [(3R)-3-methylpyrrolidin-1-yl]methyl, [(3S)-3-methylpyrrolidin-1-yl]methyl, [(2S)-2-methylpyrrolidin-1-yl]methyl, 5-azaspiro[2.4]heptan-5-ylmethyl, 2-azaspiro[3.3]heptan-2-ylmethyl, and aminomethyl.

In some embodiments, T represents a (C₁-C₄)alkylene-ORᵢ group. Advantageously, T represents a group selected from: methoxymethyl, ethoxymethyl, and cyclopropylmethoxymethyl.

Preferably, the compound according to E1 is selected from the following list:
5-[2-benzylsulfonyl-7-[(3R)-3-methyl-3,4-dihydro-1H-isoquinoline-2-carbonyl]-3,4-dihydro-1H-isoquinolin-6-yl]-N-(5-cyano-1,2-dimethyl-pyrrol-3-yl)-N-(4-hydroxyphenyl)-1,2-dimethyl-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(morpholin-4-yl)ethoxy]phenyl}acetyl)-1,2,3,4-tetrahydroisoquinolin-6-yl}-1H-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[4-(2,2,2-trifluoroethyl)piperazin-1-yl]phenyl}acetyl)-1,2,3,4-tetrahydroisoquinolin-6-yl}-1H-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(oxan-4-yl)ethoxy]phenyl}acetyl)-1,2,3,4-tetrahydroisoquinolin-6-yl}-1H-pyrrole-3-carboxamide,
N-(5-cyano-1,2-dimethylpyrrol-3-yl)-5-[2-(2-{3-fluoro-4-[1-(2-fluoroethyl)piperidin-4-yl]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide,
N-(5-cyano-1,2-dimethylpyrrol-3-yl)-5-{2-[2-(2-fluoro-4-methoxyphenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide,
N-(5-cyano-1,2-dimethylpyrrol-3-yl)-5-[2-(2-{2-fluoro-4-[2-(oxan-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide,
N-(5-cyano-1,2-dimethyl-1H-pyrrol-3-yl)-5-{2-[2-(4-{2-[cis-2,6-dimethylmorpholin-4-yl]ethoxy}phenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide,
N-(5-cyano-1,2-dimethylpyrrol-3-yl)-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide,
5-[2-(2-{4-[(9aS)-octahydropyrazino[2,1-c][1,4]oxazin-8-yl]-2-fluorophenyl}acetyl)-7-[(3R)-3-methyl-1,2,3,4-tetrahydroisoquinoline-2-carbonyl]-1,2,3,4-tetrahydroisoquinolin-6-yl]-N-(5-cyano-1,2-dimethyl-1H-pyrrol-3-yl)-N-(4-hydroxyphenyl)-1,2-dimethyl-1H-pyrrole-3-carboxamide,
N-(5-cyano-1,2-dimethylpyrrol-3-yl)-5-[2-(2-{2-fluoro-4-[2-(oxan-4-yl)ethoxy]phenyl}acetyl)-7-{[(3S)-3-(methoxymethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide,
N-(5-cyano-1,2-dimethyl-pyrrol-3-yl)-5-[2-[2-[2-fluoro-4-(2-tetrahydropyran-4-ylethoxy)phenyl]acetyl]-7-[(3S)-3-(morpholinomethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(thiomorpholin-4-yl)ethoxy]phenyl}acetyl)-1,2,3,4-tetrahydroisoquinolin-6-yl}-1H-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-((2R)-2-methylmorpholin-4-yl)ethoxy]phenyl}acetyl)-1,2,3,4-tetrahydroisoquinolin-6-yl}-1H-pyrrole-3-carboxamide.
5-{2-benzoyl-7-[(3R)-3-methyl-1,2,3,4-tetrahydroisoquinoline-2-carbonyl]-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide,
N-(5-cyano-1,2-dimethylpyrrol-3-yl)-N-(4-hydroxyphenyl)-1,2-dimethyl-5-[2-(2-{4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]pyrrole-3-carboxamide,
N-[5-cyano-1-(difluoromethyl)-2-methylpyrrol-3-yl]-N-(4-hydroxyphenyl)-1,2-dimethyl-5-[2-(2-{4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]pyrrole-3-carboxamide,
N-[5-cyano-1-(difluoromethyl)-2-methylpyrrol-3-yl]-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide,
N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[methyl(propan-2-yl)amino]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide,
N-(5-cyano-1,2-dimethylpyrrol-3-yl)-5-{2-[2-(4-{2-[cyclopropyl(methyl)amino]ethoxy}-2-fluorophenyl)acetyl]-7-{[(3S)-3-(pyrrolidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide,
N-(5-cyano-1,2-dimethyl-1H-pyrrol-3-yl)-5-[2-(2-{2-fluoro-4-[2-((2R or S)-2-methylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide,
N-[5-cyano-1-(difluoromethyl)-2-methylpyrrol-3-yl]-N-(4-hydroxyphenyl)-1,2-dimethyl-5-[2-(2-{2-fluoro-4-[2-(morpholin-4-yl)ethoxy]phenyl}acetyl)-7-{[(3S)-3-(pyrrolidin-1-ylmethyl)-3,4-dihydro-1H-isoquinolin-2-yl]carbonyl}-3,4-dihydro-1H-isoquinolin-6-yl]pyrrole-3-carboxamide,
N-(5-cyano-1,2-dimethylpyrrol-3-yl)-5-[2-(2-{4-[2-(4,4-difluoropiperidin-1-yl)ethyl]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide,
N-(5-cyano-1,2-dimethyl-1H-pyrrol-3-yl)-5-[2-(2-{4-[2-(4-fluoropiperidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide,
N-(5-cyano-1,2-dimethylpyrrol-3-yl)-N-(4-hydroxyphenyl)-1,2-dimethyl-5-[2-(2-{4-[2-(oxan-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]pyrrole-3-carboxamide,
N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(3S)-3-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide.

The invention relates also to a process for the preparation of a compound of formula (I) characterised in that there is used as starting material the compound of formula (II):

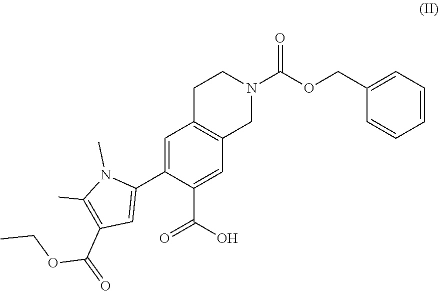 [SMILES: CCOC(=O)c1cc(-c2cc3c(cc2C(=O)O)CN(C(=O)OCc2ccccc2)CC3)n(C)c1C]

which is subjected to peptide coupling with a compound of formula (III):

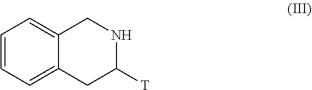

wherein T is as defined in formula (I),
to yield the compound of formula (IV):

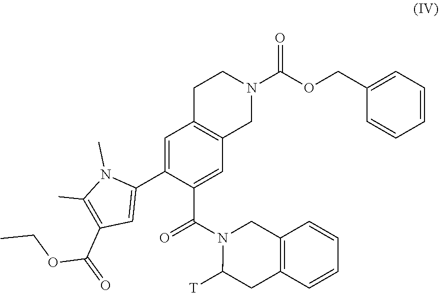

wherein T is as defined in formula (I),
the carboxybenzyl (Cbz) protecting group of which compound of formula (IV) is then replaced with a tert-butyloxycarbonyl (Boc) group before hydrolising the ester function of the pyrrole moiety to yield the compound of formula (V):

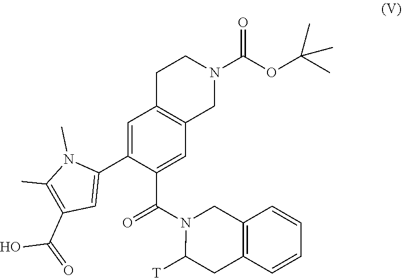

wherein T is as defined in formula (I),
compound of formula (V) which is coupled with the following amine:

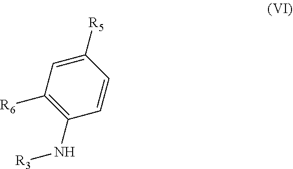

wherein R₃, R₅ and R₆ are as defined in formula (I),
to yield the compound of formula (VII):

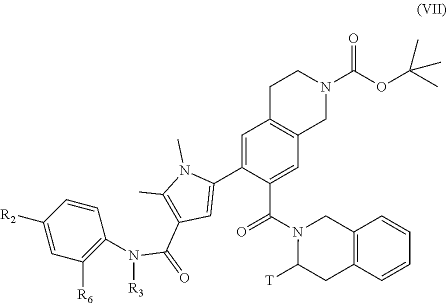

wherein T, R₃, R₅ and R₆ are as defined in formula (I),
which compound of formula (VII) is deprotected and subjected to an amidation or a sulfonylation reaction to yield the compound of formula (I), this latter compound may be purified according to a conventional separation technique, may be converted into its addition salts with a pharmaceutically acceptable acid or base and may be separated into its isomers according to a conventional separation technique,
it being understood that at any moment considered appropriate during the course of the process described above, some groups (hydroxy, amino) of the starting reagents or of the synthesis intermediates can be protected and subsequently deprotected, as required by the synthesis.

In another embodiment, the invention relates to a process for the preparation of a compound of formula (I) characterised in that there is used as starting material the compound of formula (II):

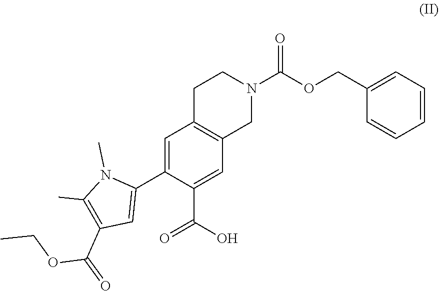 [SMILES: CCOC(=O)c1cc(-c2cc3c(cc2C(=O)O)CN(C(=O)OCc2ccccc2)CC3)n(C)c1C]

the ester function of which compound of formula (II) is hydrolysed to yield the corresponding carboxylic acid, which is then subjected to a reaction with 1,1-di-tert-butoxy-N,N-dimethyl-methanamine to yield the compound of formula (VIII):

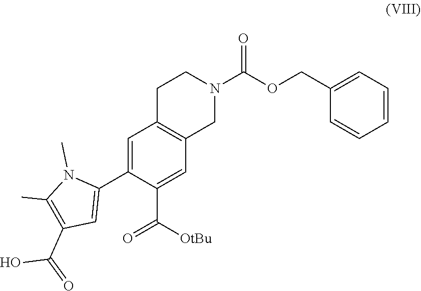 [SMILES: Cc1c(C(=O)O)cc(-c2cc3c(cc2C(=O)OC(C)(C)C)CN(C(=O)OCc2ccccc2)CC3)n1C]

wherein tBu represents a tert-butyl group,
compound of formula (VIII) which is coupled with the amine of formula (VI):

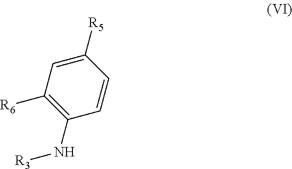

wherein R₃, R₅ and R₆ are as defined in formula (I),
to yield the compound of formula (IX):

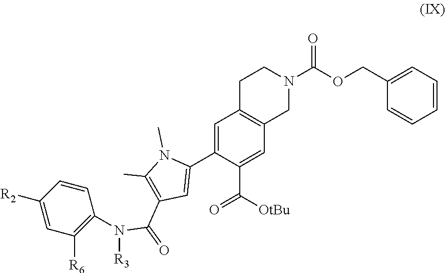

wherein R₃, R₅ and R₆ are as defined in formula (I),
the carboxybenzyl protecting group of which compound of formula (IX) being removed by a catalytic hydrogenation reaction to yield the compound of formula (X):

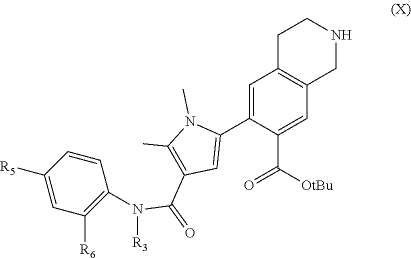

wherein R₃, R₅ and R₆ are as defined in formula (I),
which compound is subjected to an amidation or a sulfonylation reaction to yield the compound of formula (XI):

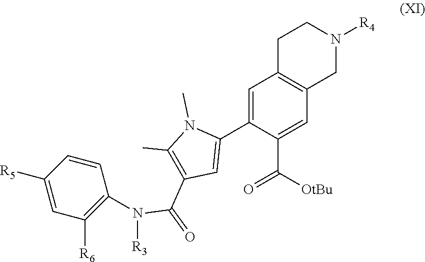

wherein R₃, R₄, R₅ and R₆ are as defined in formula (I),
the tert-butyl ester function of which compound of formula (XI) being deprotected, before being subjected to peptide coupling with a compound of formula (III):

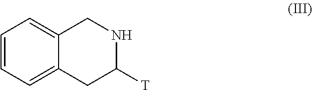

wherein T is as defined in formula (I),
to yield the compound of formula (I), this latter compound may be purified according to a conventional separation technique, may be converted into its addition salts with a pharmaceutically acceptable acid or base and may be separated into its isomers according to a conventional separation technique,
it being understood that at any moment considered appropriate during the course of the process described above, some groups (hydroxy, amino) of the starting reagents or of the synthesis intermediates can be protected and subsequently deprotected, as required by the synthesis.

Pharmacological studies of the compounds of the invention have shown that they have pro-apoptotic properties. The ability to reactivate the apoptotic process in cancerous cells is of major therapeutic interest in the treatment of cancer and of immune and auto-immune diseases. In particular, the compounds according to the invention will be useful in the treatment of chemo- or radio-resistant cancers.

In some embodiments of the present disclosure, the compounds of the invention are very potent on the BCL2 wild type protein (i.e. with a EKI_BCL2 wild type value lower than 1 nM in the TR-FRET assay) and on the BCL2 G101V mutant (i.e. with a [EKI BCL2 G101V]/[EKI BCL2 wild type] selectivity ratio lower than 10, more preferably lower than 5) whilst showing a limited on-target affinity for BCL-XL (i.e. with a [EKI BCL-XL]/[EKI BCL2 wild type] selectivity ratio higher than 10). In a further embodiment, the compounds of the invention also exhibit an overall better on-target affinity for the Bcl-2 wild type protein and the Bcl-2 mutants as compared to Bcl-xL protein. In some embodiments, the Bcl-2 mutants (also called BCL2 mutated proteins) are selected from the following group: BCL2 G101V, BCL2 D103Y, BCL2 F104I, BCL2 D111A, BCL2 D103E, BCL2 A113G, BCL2 R129L, BCL2 V156D, BCL2 D103V, and BCL2 F104L.

In some embodiments of the present disclosure, the compounds of the invention are highly potent on the BCL2 wild type protein (i.e. with a EKI BCL2 wild type value lower than 100 pM in the TR-FRET assay) whilst showing a limited on-target affinity for BCL-XL (i.e. with a [EKI BCL-XL]/[EKI BCL2 wild type] selectivity ratio higher than 100).

The present invention relates also to pharmaceutical compositions comprising at least one compound of formula (I) or an addition salt thereof with a pharmaceutically acceptable acid or base in combination with one or more pharmaceutically acceptable excipients. In particular, these pharmaceutical compositions are interesting for use as anti-apoptotic inhibitors, particularly, in the treatment of cancer (haematological malignancy and solid tumor) and of auto-immune and immune system diseases. Particularly, these pharmaceutical compositions are interesting for use as anti-apoptotic inhibitors in the treatment of cancer chemo-resistant or radio-resistant. Preferably, these pharmaceutical compositions can be used in the treatment of cancer (haematological malignancy and solid tumor) and of auto-immune and immune system diseases selected from myeloma, especially multiple myeloma, lymphoma, especially Non-Hodgkin Lymphoma (NHL) and Diffuse Large B-cell Lymphoma (DLBCL), leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML), bladder, brain, breast, uterus, oesophagus and liver cancers, colorectal cancer, renal cancer, melanoma, ovarian cancer, prostate cancer, pancreatic cancer, lung cancer, especially non-small-cell lung cancer and small-cell lung cancer, rheumatoid arthritis (RA) or systemic lupus erythematosus (SLE). In some embodiments, the compounds of formula (I) are used in the manufacture of medicaments for use in the treatment of cancer and of auto-immune and immune system diseases.

Furthermore, the present invention relates also to the combination of a compound of formula (I) with an anticancer agent selected from genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies, and also to pharmaceutical compositions comprising that type of combination and their use in the manufacture of medicaments for use in the treatment of cancer, particularly, haematological malignancy and solid tumors selected from myeloma, especially multiple myeloma, lymphoma, especially Non-Hodgkin Lymphoma (NHL) and Diffuse Large B-cell Lymphoma (DLBCL), leukemia, especially Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML), bladder, brain, breast, uterus, oesophagus and liver cancers, colorectal cancer, renal cancer, melanoma, ovarian cancer, prostate cancer, pancreatic cancer and lung cancer, especially non-small-cell lung cancer and small-cell lung cancer.

In another aspect, the compounds of the invention can be used in combination with radiotherapy in the treatment of cancer.

Alternatively, the compounds of the invention may be linked to monoclonal antibodies. Antibody Drug Conjugates (ADCs) represent a new class of therapeutics that is formed by chemically linking a cytotoxic drug to a monoclonal antibody through a linker. The monoclonal antibody of an ADC selectively binds to a target antigen of a cell (e.g. cancer cell) and releases the drug into the cell. ADCs have therapeutic potential because they combine the specificity of the antibody and the cytotoxic potential of the drug. Nonetheless, developing ADCs as therapeutic agents has thus far met with limited success owing to a variety of factors such as unfavorable toxicity profiles, low efficacies and poor pharmacological parameters. Accordingly, there is still a need for new ADCs that overcome these problems and can selectively deliver Bcl-2 to target cancer cells.

In another aspect, the compounds of the invention may be linked to fragments of monoclonal antibodies or linked to scaffold proteins that can be related or not to monoclonal antibodies. Antibody fragments must be understood as fragments of Fv, scFv, Fab, F(ab′)2, F(ab′), scFv-Fc type or diabodies, which generally have the same specificity of binding as the antibody from which they are descended. According to the present invention, antibody fragments of the invention can be obtained starting from antibodies by methods such as digestion by enzymes, such as pepsin or papain, and/or by cleavage of the disulfide bridges by chemical reduction. In another manner, the antibody fragments comprised in the present invention can be obtained by techniques of genetic recombination likewise well known to the person skilled in the art or else by peptide synthesis by means of, for example, automatic peptide synthesizers such as those supplied by the company Applied Biosystems, etc.

Scaffold proteins that can be related or not to monoclonal antibodies are understood to mean a protein that contains or not an immunoglobulin fold and that yields a binding capacity similar to a monoclonal antibody. The person skilled in the art knows how to select the protein scaffold. More particularly, it is known that, to be selected, such a scaffold should display several features as follow (Skerra, J. Mol. Recogn. 2000, 13, 167-187): phylogenetically good conservation, robust architecture with a well-known three-dimensional molecular organization (such as, for example, crystallography or NMR), small size, no or only a low degree of post-translational modifications, easy to produce, express and purify. Such a protein scaffold can be, but without limitation, a structure selected from the group consisting in fibronectin and preferentially the tenth fibronectin type III domain (FNfn10), lipocalin, anticalin (Skerra, J. Biotechnol. 2001, 74, 257-75), the protein Z derivative from the domain B of staphylococcal protein A, thioredoxin A or any protein with a repeated domain such as an “ankyrin repeat” (Kohl et al. PNAS 2003, 100, 1700-1705), “armadillo repeat”, “leucine-rich repeat” or “tetratricopeptide repeat”. There could also be mentioned a scaffold derivative from toxins (such as, for example, scorpion, insect, plant or mollusc toxins) or protein inhibitors of neuronal nitric oxide synthase (PIN).

The following Examples illustrate the invention but do not limit it in any way. All the reagents for preparing Examples are either commercially available or can be obtained by the person skilled in the art using conventional chemical reactions described in the literature.

### General Synthetic Remarks

All reagents obtained from commercial sources were used without further purification. Anhydrous solvents were obtained from commercial sources and used without further drying.

The reactions were monitored using LCMS and GCMS instruments and/or TLC.

### TLC

Thin layer chromatography was conducted with 5×10 cm plates coated with Merck Type 60 F254 silica-gel.

### Column Chromatography

Automated flash column chromatography was performed on ISCO CombiFlash® Rf 200 or CombiFlash® Rf+ Lumen™ using RediSep® RfNormal-phase Silica Flash Columns (35-70 μm, 60 Å), RediSep Rf Gold® Normal-phase Silica High Performance Columns (20-40 μm, 60 Å), RediSep® Rf Reversed-phase C18 Columns (40-63 μm, 60 Å), or RediSep Rf Gold® Reversed-phase C18 High Performance Columns (20-40 μm, 100 Å). For HILIC RediSep Rf Gold® Normal-phase Silica High Performance Columns (20-40 μm, 60 Å) were used.

### Microwave Reactions

Microwave heating was performed with a CEM Discover® SP, or with an Anton Paar Monowave Microwave Reactor.

### NMR

¹H-NMR and ¹³C-NMR measurements were performed on Bruker Avance III 500 MHz spectrometer, Bruker Avance III 400 MHz spectrometer, Bruker DPX 400 MHz spectrometer and Bruker Avance NEO 400 MHz spectrometer, using DMSO-d₆ or CDCl₃ as solvent. ¹H NMR data is in the form of delta values, given in part per million (ppm), using the residual peak of the solvent (2.50 ppm for DMSO-d₆ and 7.26 ppm for CDCl₃) as internal standard. Splitting patterns are designated as: s (singlet), d (doublet), t (triplet), q (quartet), quint (quintet), sept (septet), m (multiplet), br s (broad singlet), br d (broad doublet), br t (broad triplet), br m (broad multiplet), dd (doublet of doublets), td (triplet of doublets), dt (doublet of triplets), qd (quartet of doublets), ddd (doublet of doublet of doublets), dm (doublet of multiplets). In certain cases—due to the complexity—only the decisive signals of the carbon spectra were interpreted.

### Analytical GC-MS

Combination gas chromatography and low-resolution mass spectrometry (GC-MS) was performed on Agilent 6850 gas chromatograph and Agilent 5975C mass spectrometer using 15 m×0.25 mm column with 0.25 μm HP-5MS coating and helium as carrier gas. Ion source: EI+, 70 eV, 230° C., quadrupole: 150° C., interface: 300° C.

### Analytical LC-MS

The compounds of the present invention were characterized by high performance liquid chromatography-mass spectroscopy (HPLC-MS) using the following instruments:

Agilent HP1200 LC with Agilent MSD 6140 single quadrupole, operating in positive or negative ion electrospray ionisation mode. Molecular weight scan range is 100 to 1350 m/z. Parallel UV detection was done at 210 nm and 254 nm. Samples were supplied as a 1 mM solution in MeCN, or in THF/water (1:1) with 5 μL loop injection. LCMS analyses were performed on 2 instruments, one of which was operated with basic, and the other with acidic eluents.

Basic LCMS: Gemini-NX, 3 μm, C18, 50 mm×3.00 mm i.d. column at 23° C., at a flow rate of 1 mL min⁻¹ using 5 mM aq. NH₄HCO₃ solution (Solvent A) and MeCN (Solvent B) with a gradient starting from 100% Solvent A and finishing at 100% Solvent B over various duration of time.

Acidic LCMS: ZORBAX Eclipse XDB-C18, 1.8 μm, 50 mm×4.6 mm i.d. column at 40° C., at a flow rate of 1 mL min⁻¹ using 0.02% V/V aq. HCOOH solution (Solvent A) and 0.02% V/V HCOOH solution in MeCN (Solvent B) with a gradient starting from 100% Solvent A and finishing at 100% Solvent B over various duration of time.

Agilent HP1260 Infinity II LC with InfinityLab LC/MSD, operating in positive ion electrospray ionisation mode. Molecular weight scan range is 100 to 1350 m/z. Parallel UV detection was done at 210 nm and 254 nm. Samples were supplied as a 1 mM solution in MeCN, or in methanol with 1-5 μL loop injection operated with Kinetex® XB-C18, 2.6 μm, 50 mm×2.1 mm i.d. column at 40° C., at a flow rate of 1 mL min⁻¹ using 0.05% V/V TFA solution in 20/1 water/MeCN (Solvent A) and 0.05% V/V TFA solution in 1/20 water/MeCN (Solvent B) with a gradient starting from 100% Solvent A and finishing at 100% Solvent B over various duration of time.

Agilent HP1200 LC with Agilent MSD 6120 Quadrupole LC/MS, operating in positive or negative ion electrospray ionisation mode. Molecular weight scan range is 100 to 1350 m/z. Parallel UV detection was done at 210 nm and 254 nm. Samples were supplied as a 1 mM solution in MeCN, or in methanol with 1-5 μL loop injection operated with Gemini® NX-C18, 3 μm, 50 mm×3 mm i.d. column at 40° C., at a flow rate of 0.8 mL min⁻¹ using 5 mM NH₄HCO₃ solution in 20/1 water/MeCN (Solvent A) and MeCN (Solvent B) with a gradient starting from 100% Solvent A and finishing at 90% Solvent B over various duration of time.

### High-Resolution MS

Agilent 1200 SL series instrument linked to an Agilent MSD 6140 single quadrupole with an ESI-APCI multimode source or using an Agilent 1290 Infinity II series instrument connected to an Agilent TOF 6230 with an ESI-jet stream source; column: Thermo Accucore 2.6 μm, C18, 50 mm×2.1 mm at 55° C. or Agilent Zorbax Eclipse plus 3.5 μm, C18, 30 mm×2.1 mm at 35° C.; eluents: Solvent A: 10 mM aq. NH₄OAc solution+0.08% (v/v) HCOOH; Solvent B: MeCN+5% (v/v) Solvent A+0.08% (v/v) HCCOH, with a gradient starting from 95% Solvent A and finishing at 95% Solvent B over various duration of time; ionisation is recorded in positive mode, negative mode, or positive-negative switching mode.

TOF (basic): Agilent 6230 time-of-flight mass spectrometer equipped with a Jet Stream electrospray ion source in positive ion mode. Injections of 0.5 μl were directed to the mass spectrometer at a flow rate 0.7 ml/min (5 mM ammonium-bicarbonate in water and acetonitrile gradient program), using an Agilent 1290 Infinity HPLC system. Jet Stream parameters: drying gas (N2) flow and temperature: 8.0 l/min and 325° C., respectively; nebulizer gas (N2) pressure: 30 psi; capillary voltage: 3000 V; sheath gas flow and temperature: 325° C. and 10.0 l/min; TOFMS parameters: fragmentor voltage: 100 V; skimmer potential: 60 V; OCT 1 RF Vpp: 750 V. Full-scan mass spectra were acquired over the m/z range 105-1700 at an acquisition rate of 995.6 ms/spectrum and processed by Agilent MassHunter B.08.00/10.10 software.

Q-TOF (neutral): Agilent 6545 quadrupole-time-of-flight mass spectrometer equipped with a Duo-Jet Stream electrospray ion source in positive or negative ion mode. Injections of 0.5 μl were directed to the mass spectrometer at a flow rate 0.7 ml/min (5 mM ammonium-formate in water and acetonitrile gradient program), using an Agilent 1290 Infinity HPLC system. Jet Stream parameters: drying gas (N2) flow and temperature: 10.0 l/min and 300° C., respectively; nebulizer gas (N2) pressure: 40 psi; capillary voltage: 2500 V; sheath gas flow and temperature: 300° C. and 10.0 l/min; TOFMS parameters: fragmentor voltage: 100 V; skimmer potential: 65 V; OCT 1 RF Vpp:750 V. Full-scan mass spectra were acquired over the m/z range 105-1700 at an acquisition rate of 1000.0 ms/spectrum and processed by Agilent MassHunter B.08.00/10.10 software.

### Preparative HPLC

Certain compounds of the present invention were purified by high performance liquid chromatography (HPLC) on the following instruments:

Armen Spot Prep II Liquid Chromatography or Teledyne CombiFlash EZ Prep system with a Gemini NX® 5 pM C18, 250 mm×50 mm i.d. column running at a flow rate of 118 mL min-1 with UV diode array detection (210-400 nm) using 25 mM aqueous NH₄HCO₃ solution and MeCN or 0.1% TFA in water and MeCN as eluents.

Waters FractionLynx MS autopurification system, with a Gemini® 5 μm C18(2), 100 mm×20 mm i.d. column from Phenomenex, running at a flow rate of 20 mL min⁻¹ with UV diode array detection (210-400 nm) and mass-directed collection. The mass spectrometer was a Waters Micromass ZQ2000 spectrometer, operating in positive or negative electrospray ionisation modes, with a molecular weight scan range of 150 to 1000. pH4 eluents: Solvent A: 10 mM aq. NH₄OAc solution+0.08% (v/v) HCOOH; Solvent B: MeCN+5% (v/v) Solvent A+0.08% (v/v) HCOOH. pH9 eluents: Solvent A: 10 mM aq. NH₄OAc solution+0.08% (v/v) cc. aq. NH₃ solution; Solvent B: MeCN+5% (v/v) Solvent A+0.08% (v/v) cc. aq. NH₃ solution.

AccQPrep HP125 (Teledyne ISCO) system, with a Gemini® NX 5 μm C18(2), 150 mm×21.2 mm i.d. column from Phenomenex, running at a flow rate of 20 mL min⁻¹ or Gemini® NX 5 μm C18(2), 250 mm×30 mm i.d. column from Phenomenex, running at a flow rate of 40 mL min⁻¹ with UV (214 and 254 nm) and ELS detection. pH4 eluents: Solvent A: water+0.08% (v/v) HCOOH; solvent B: MeCN+0.08% (v/v) HCOOH. pH9 eluents: Solvent A: water+0.08% (v/v) cc. aq. NH₃ solution; solvent B: MeCN+0.08% (v/v) cc. aq. NH₃ solution. Neutral eluents: Solvent A: water; Solvent B: MeCN.

### Chemical Naming

IUPAC-preferred names were generated using ChemAxon's ‘Structure to Name’ (s2n) functionality within MarvinSketch or JChem for Excel (JChem versions 16.6.13-18.22.3), or with the chemical naming functionality provided by Biovia® Draw 4.2.

### Abbreviations

AcOH acid acetic
ACN acetonitrile
aq. aqueous
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate
Boc or tBu ester tert-butyloxycarbonyl
^{t}BuOH tert-butanol
Cbz carboxybenzyl
cc. concentrated
DAST diethylaminosulfur trifluoride
DavePhos 2-dicyclohexylphosphino-2″-(N,N-dimethylamino)biphenyl
DBU 1,8-diazabicyclo[5.4.0]undec-7-ene
DCE 1,2-dichloroethane
DCM dichloromethane
DIPEA diisopropylethylamine
DMF N,N-dimethylformamide
DTBAD di-tert-butyl azodicarboxylate
ee. enatiomeric excess
eq. equivalent
EtOAc ethyl acetate
Fmoc fluorenylmethoxycarbonyl
HATU 1-[bis(dimethylamino)methylene]-1H-1,2,3-triazolo[4,5-b]pyridinium 3-oxid hexafluorophosphate
hu human
IPA, i-PrOH or 2-PrOH Isopropyl alcohol
MeCN acetonitrile
MTBE methyl tert-butyl ether
Pd₂dba₃ tris(dibenzylideneacetone)dipalladium(0)
Pd(dppf)Cl₂ DCM [1,1′-bis(diphenylphosphino)ferrocene]dichloropalladium(II), complex with dichloromethane
PTFE polytetrafluoroethylene
PyBop [(1H-benzotriazol-1-yl)oxy][tri(pyrrolidin-1-yl)]phosphonium hexafluorophosphate
quant. quantitative
rt room temperature
sat. saturated
TBAF tetrabutyl ammonium fluoride
TBDMSCl tert-butyldimethylsilyl chloride
TBTU [benzotriazol-1-yloxy(dimethylamino)methylene]-dimethyl-ammonium, tetrafluoroborate (1:1)
TEA triethylamine
TFA trifluoroacetic acid
THE tetrahydrofuran
THIQ 1,2,3,4-tetrahydroisoquinoline
TIPSCl triisopropylsilyl chloride
X-Phos 2-(Dicyclohexylphosphino)-2′,4′,6′-triisopropylbiphenyl

### GENERAL PROCEDURES

The following are representative experimental procedures that are referred to by name in subsequent Preparations and Examples.

General Procedure 1a-Buchwald

The appropriate aryl bromide (1 eq.), the appropriate aniline (1.1 eq.), 2-di-tert-butylphosphino-2′,4′,6′-triisopropylbiphenyl (0.04 eq.) and NaO^{t}Bu (2 eq.) were suspended in toluene (3 mL/mmol aryl bromide). The mixture was sparged with N₂ and then Pd₂(dba)₃ (0.04 eq.) was added, and the mixture was heated for 1 h at 110° C. under microwave irradiation or at 70° C. under a N₂ atmosphere until complete conversion was observed. The mixture was allowed to cool to rt, diluted with water and filtered through a celite cartridge. The filtrate was partitioned between EtOAc and water. The phases were separated, and the aqueous phase was extracted with EtOAc. The combined organic phases were dried over MgSO₄ and concentrated in vacuo. The crude material was purified by automated flash chromatography using heptane and EtOAc as eluents.

General Procedure 1b-Buchwald

To a solution of the appropriate aryl bromide (1 eq.) and the appropriate aniline (1 eq.) in THE (4.5 mL/mmol aryl bromide) was added NaO^{t}Bu (1 eq.) and chloro(2-di-t-butylphosphino-2′,4′,6′-tri-i-propyl-1,1′-biphenyl)[2-(2-aminoethyl)phenyl]palladium(II) [t-BuXPhos Palladacycle Gen. 1] (0.04 eq.) and the mixture was stirred at rt under N₂ until complete conversion was observed. The mixture was diluted with water and the organic layer was extracted with EtOAc. The combined organic layers were washed with brine, dried over MgSO₄ and concentrated in vacuo. The crude material was purified by automated flash chromatography using heptane or MeOH and EtOAc as eluents.

General Procedure 2a—Amide Coupling

To a solution of the appropriate acid (1 eq.) in DCE (12 mL/mmol acid) was added 4 Å molecular sieves followed by 1-chloro-N,N,2-trimethyl-1-propenylamine (2 eq.) and then the mixture was stirred under N₂ at rt for 30 mins. A solution of the appropriate aniline (1.5 eq.) and pyridine (2 eq.) in DCE (2.5 mL/mmol aniline) was added and the mixture stirred at 80° C. under N₂ for 18 h. The mixture was allowed to cool to rt and partitioned between DCM and water. The phases were separated, and the organic phase was washed with sat. aq. NaHCO₃ solution, dried over MgSO₄ and concentrated in vacuo. The crude material was purified by automated flash chromatography using heptane and EtOAc or DCM and MeOH as eluents.

General Procedure 2b—Amide Coupling

To a solution of the appropriate acid (1 eq.) in DCM (12 mL/mmol acid) was added oxalyl dichloride (1.7 eq.) and then the mixture was stirred under N₂ at rt for 30 mins. The solution was concentrated in vacuo at rt then the appropriate aniline (1.2 eq.) in DCE (12 mL/mmol acid) was added and the mixture was stirred at 80° C. under N₂ for 4-18 h. The reaction mixture was concentrated in vacuo and the crude material was purified by automated flash chromatography using DCM and MeOH as eluents.

General Procedure 3a—tBu Ester Deprotection

To a solution of the appropriate tBu ester (1 eq.) in 1,4-dioxane (5 mL/mmol) was added a solution of trimethylsilyl trifluoromethanesulfonate (5 eq.) and TEA (5 eq) in 1,4-dioxane (1 mL/mmol TEA) and the mixture stirred at rt for 5-18 h. The reaction mixture was poured into ice-water, stirred for 10 mins, then extracted with EtOAc. The organic phase was dried over MgSO₄ and concentrated in vacuo. The crude material was purified by automated flash chromatography using DCM and MeOH as eluents.

General Procedure 3b—HCl Deprotection

To a solution of the appropriate silyl-derivative in MeOH (5 mL/mmol) was added a solution of 3 M HCl in MeOH (5 mL/mmol silyl derivative) and the mixture stirred at rt until complete conversion was observed. The mixture was concentrated in vacuo and then partitioned between DCM and sat. aq. NaHCO₃ solution. The phases were separated, and the organic phase was dried over MgSO₄ and concentrated in vacuo. The crude material was purified by automated flash chromatography using DCM and MeOH as eluents or preparative HPLC automated flash chromatography at pH 9 or pH 4, using water and MeCN as eluents.

General Procedure 3c—TBAF Deprotection

To a solution of the appropriate silyl-derivative (1 eq.) in THE (50 mL/mmol) under N₂ was added 1 M TBAF in THE (2 eq.) and the mixture was stirred at rt for 18 h. The mixture was partitioned between DCM and sat. aq. NaHCO₃ solution and then the phases were separated. The aqueous phase was extracted with DCM and the combined organic extracts were dried over MgSO₄ and concentrated in vacuo. Purification by automated flash chromatography using DCM and MeOH as eluents was followed by preparative HPLC automated flash chromatography at pH 4 or pH 9, using water and MeCN as eluents.

General Procedure 4a—Amide Coupling

To a solution of the appropriate amine (1 eq.) in DMF (6 mL/mmol amine) was added the appropriate carboxylic acid (1.5 eq.) followed by DIPEA (2 eq.) and HATU (1 eq.) and the mixture was stirred at rt until complete conversion was observed. The reaction mixture was partitioned between DCM and water. The phases were separated and the organic phase dried over MgSO₄ and concentrated. Purification by automated flash chromatography using DCM and MeOH as eluents was followed by preparative HPLC automated flash chromatography at pH 9 or pH 4, using water and MeCN as eluents.

General Procedure 4b—Amide Coupling

To a solution of the appropriate amine (1 eq.) in DMF (6 mL/mmol amine) was added the appropriate carboxylic acid (1.2 eq.) followed by DIPEA (4 eq.) and PyBop (1.2 eq.) and the mixture was stirred at rt until complete conversion was observed. DMF was removed in vacuo and the residue was partitioned between DCM and water. The phases were separated and the organic phase dried over MgSO₄ and concentrated in vacuo. The crude material was purified by preparative HPLC automated flash chromatography at pH 9 or pH 4, using water and MeCN as eluents.

General Procedure 4c—Consecutive Amide Coupling and Silyl Deprotection

To a solution of the carboxylic acid (1.5 eq.) in DMF (6 mL/mmol acid) was added TBTU (3 eq.) followed by DIPEA (4 eq.). After stirring for 10 mins the appropriate amine (1 eq.) was added and the mixture was stirred at rt until complete conversion was observed. 2M aq. NaOH solution (5 eq.) was added and the reaction mixture was further stirred until complete silyl deprotection was observed. The reaction mixture—after filtration—was directly injected to an RP C18 column and it was purified via preparative reversed phase chromatography. The appropriate fractions were combined, concentrated under reduced pressure to approximately 25 mL then extracted with DCM (2×20 mL). The combined organic extracts were dried over Na₂SO₄ and concentrated to furnish the targeted amide product which was further purified by automated flash chromatography using DCM and MeOH as eluents.

General Procedure 4d—Consecutive Amide Coupling and Silyl Deprotection

Modified General procedure 4c: carboxylic acid chloride (1.2 eq.), DIPEA or TEA (2 eq.) in DCM (3 mL/mmol) were used in the place of carboxylic acid (1.5 eq.), DIPEA (4 eq.) and TBTU were omitted.

### General Procedure 5—THIQ Amide Coupling

HATU (1.1 eq.) was added portionwise to a stirred solution of the appropriate amine (1.05 eq.), Boc-(3S)-1,2,3,4-tetrahydroisoquinoline-3-carboxylic acid (1 eq.), and TEA (2 eq.) in DMF (10 mL/mmol) under N₂. The mixture was stirred at rt for 12 h, and then partitioned between EtOAc and sat. aq. NaHCO₃ solution. The phases were separated, and the organic phase was washed with brine, dried over MgSO₄ and concentrated in vacuo. The crude material was purified by automated flash chromatography using DCM and MeOH as eluents.

General Procedure 5a—THIQ Amide Coupling

TBTU (1.5 eq.) was added portion-wise to a stirred solution of the Boc or Cbz protected (3S)-1,2,3,4-tetrahydroisoquinoline-3-carboxylic acid (1 eq.) and DIPEA (3 eq.) in DMF/DCM (5 mL/mmol acid) under N₂. After stirring for 10 mins the appropriate amine (1.4 eq.) was added and the mixture was stirred at rt until complete conversion was observed. The mixture was then partitioned between EtOAc and water. The phases were separated, and the organic phase was washed with brine, dried over Na₂SO₄ and concentrated in vacuo. The crude material was purified by automated flash chromatography using DCM and MeOH as eluents.

### General Procedure 6—THIQ Amide Reduction and Deprotection

To a solution of the appropriate THIQ amide (1 eq.) in THE (5-7 mL/mmol THIQ) was added 1 M BH₃·THF solution in THE (5 eq.) dropwise under an atmosphere of N₂ at rt. The mixture was then heated to reflux for 5 h. The mixture was allowed to cool to rt, quenched by the slow addition of MeOH and then stirred for 10 min at rt. The mixture was concentrated under reduced pressure and the residue suspended in MeOH (5-7 mL/mmol). Concentrated HCl (4 eq. (12M)) was added and the mixture was refluxed for 1.5 h. The reaction was allowed to cool to rt, concentrated under reduced pressure and azeotroped with toluene.

General Procedure 6a—THIQ Amide Reduction

A solution of the appropriate THIQ amide (1 eq.) in DCM (5-10 mL/mmol THIQ) was cooled to 0° C. with an ice-water bath, then 1M LiAlH₄ solution in THE (2 eq.) was added dropwise under an atmosphere of N₂. The cooling bath was removed then the mixture was stirred at rt until complete conversion was observed. The mixture was diluted with THF, cooled to 0° C. then quenched by the subsequent addition of water and 15% aq. NaOH solution according Fieser's workup method. Anhydrous MgSO₄ was added (5 g/mmol THIQ) then after 15 min stirring the salts were removed by filtration. The filtrate was concentrated in vacuo and the residue was purified by automated flash chromatography using heptane and IPA or DCM and MeOH as eluents (1% NH₃).

General Procedure 6b—Cbz Deprotection

To a N₂ flushed solution of the appropriate Cbz protected amine in methanol, THE or EtOAc (20 mL/mmol amine) 3-5 m/m % of Pd (10% on carbon) was added. The reaction mixture was hydrogenated at atmospheric pressure (by the aid of a H₂ balloon) until complete conversion was observed. The catalyst was filtered out and the filtrate was concentrated under reduced pressure.

General Procedure 6c—Boc Deprotection

To a N₂ flushed solution of the appropriate Boc protected amine in DCM (2.5-5 mL/mmol amine) HCl (2M solution in diethyl ether, 10 eq.) was added. The reaction mixture was stirred at rt overnight then concentrated in vacuo.

General Procedure 7a—O-Alkylation

A stirred suspension of the appropriate phenol (1 eq.) and K₂CO₃ (5 eq.) in MeCN (5-10 mL/mmol) was heated at 70° C. for 20 min. The appropriate alkyl halide (1.6 eq.) was added, and heating continued until complete conversion was observed. The mixture was allowed to cool to rt and filtered, washing with EtOAc. The filtrate was concentrated in vacuo and the residue was purified by automated flash chromatography using heptane and EtOAc or DCM and MeOH as eluents.

General Procedure 7b—Mitsunobu

To a solution of the appropriate phenol (1 eq.) in THE (5-10 mL/mmol) was added the appropriate alcohol (1 eq.) and PPh₃ (1.5 eq.) followed by DTBAD (1.5 eq.) and then stirred at rt under N₂ for 12 h. The mixture was diluted with EtOAc, washed with sat. aq. NaHCO₃ solution, dried over MgSO₄ and concentrated in vacuo. The residue was purified by automated flash chromatography using heptane and EtOAc or DCM and MeOH as eluents.

General Procedure 7c—Mitsunobu

In a pressure relief vial a solution of 4-bromo-5-methyl-1H-pyrrole-2-carbonitrile (1 eq.), the appropriate alcohol (2 eq.) and 2-(tributyl-λ5-phosphanylidene)acetonitrile (2 eq.) in toluene (2 mL/mmol) were stirred at 100° C. under N₂ for 1 h. The mixture was concentrated in vacuo and the residue was purified by automated flash chromatography using heptane and EtOAc or DCM as eluents.

General Procedure 7d—N-Alkylation

A stirred suspension of the appropriate ester of 4-(2-bromoethoxy)phenylacetic acid derivative (1 eq.) and K₂CO₃/Cs₂CO₃ (2-7 eq.) in MeCN (4-7 mL/mmol alkyl halide) and the appropriate amine (1.5-3 eq.) were heated at 80° C. until complete conversion was observed. The reaction mixture was concentrated in vacuo and the residue was purified by automated flash chromatography using heptane and EtOAc or DCM and MeOH as eluents.

### General Procedure 8—Ester Hydrolysis

A solution of the appropriate ester (1 eq.) in MeOH (3-11 mL/mmol) was treated with 2 M aq. NaOH solution (2 eq.) and stirred at rt for 24 h. The MeOH was removed in vacuo and the aqueous residue was neutralised with 2 M aq. HCl solution and then purified by reverse phase automated flash chromatography using water and MeCN as eluents.

### PREPARATIONS

The following experimental details describe the preparation of synthetic intermediates.

### Preparation I: 2-benzyloxycarbonyl-6-(4-ethoxycarbonyl-1,5-dimethyl-pyrrol-2-yl)-3,4-dihydro-1H-isoquinoline-7-carboxylic acid

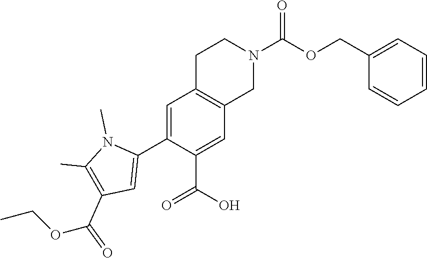 [SMILES: CCOC(=O)c1cc(-c2cc3c(cc2C(=O)O)CN(C(=O)OCc2ccccc2)CC3)n(C)c1C]

The synthesis of the title compound is described in WO2015/011164 A1, in Example 805 Step B.

### Preparation IIa: 5-[2-(tert-butoxycarbonyl)-7-{[(3R)-3-methyl-3,4-dihydro-1H-isoquinolin-2-yl]carbonyl}-3,4-dihydro-1H-isoquinolin-6-yl]-1,2-dimethylpyrrole-3-carboxylic acid

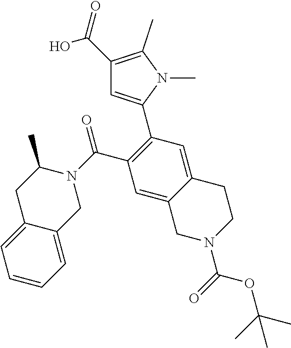 [SMILES: Cc1c(C(=O)O)cc(-c2cc3c(cc2C(=O)N2Cc4ccccc4C[C@H]2C)CN(C(=O)OC(C)(C)C)CC3)n1C]

### Step A: (3R)-3-methyl-1,2,3,4-tetrahydroisoquinoline hydrochloride

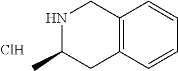

The synthesis of the title compound is described in WO2015/011164 Å1 at Preparation 2b.

### Step B: benzyl 6-[4-(ethoxycarbonyl)-1,5-dimethylpyrrol-2-yl]-7-{[(3R)-3-methyl-3,4-dihydro-1H-isoquinolin-2-yl]carbonyl}-3,4-dihydro-1H-isoquinoline-2-carboxylate

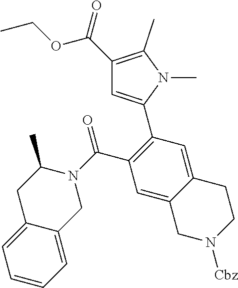 [SMILES: CCOC(=O)c1cc(-c2cc3c(cc2C(=O)N2Cc4ccccc4C[C@H]2C)CN(C(=O)OCc2ccccc2)CC3)n(C)c1C]

To a solution of Preparation I (3 g, 6.3 mmol, 1 eq.) and the product from Step A (1.27 g, 6.93 mmol, 1.1 eq.) in DMF (30 mL) was added DIPEA (3.13 mL, 18.89 mmol, 3 eq.) and PyBop (3.6 g, 6.93 mmol, 1.1 eq.) and the mixture stirred at rt for 12 h. The mixture was diluted with water (120 mL), stirred for 15 mins and the resultant cream precipitate was collected by filtration, washing with water. The filter cake was dissolved in DCM and washed with sat. aq. NaHCO₃ solution and water, dried over MgSO₄ and concentrated in vacuo to afford the title product (3.7 g, 6.11 mmol, 97%).

HRMS calcd for C₃₇H₃₉N₃O₅: 605.289, found 606.296 [M+H]⁺

¹H NMR (400 MHz, DMSO-d6) δ ppm: 7.52-6.75 (m, 11H), 6.43-6.00 (m, 1H), 5.39-1.95 (m, 21H), 1.31-0.49 (m, 6H).

### Step C: ethyl 1,2-dimethyl-5-(7-{[(3R)-3-methyl-3,4-dihydro-1H-isoquinolin-2-yl]carbonyl}-1,2,3,4-tetrahydroisoquinolin-6-yl)pyrrole-3-carboxylate

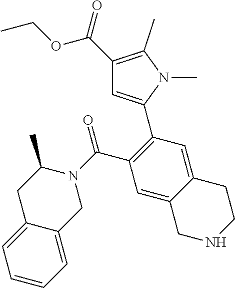 [SMILES: CCOC(=O)c1cc(-c2cc3c(cc2C(=O)N2Cc4ccccc4C[C@H]2C)CNCC3)n(C)c1C]

To a solution of the product from Step B (3.7 g, 6.11 mmol, 1 eq.) in MeOH (70 mL) and EtOH (20 mL) was added 10% Pd/C (100 mg). The mixture was evacuated and backfilled with N₂, then evacuated and flushed with H₂ and then shaken at rt for 6 h under an atmosphere of H₂. The mixture was filtered through a celite cartridge, washing with EtOH. The solvent was removed in vacuo to afford the title product (2.94 g, quant.).

HRMS calcd for C₂₉H₃₃N₃O₃: 471.252, found 472.259 [M+H]⁺

¹H NMR (400 MHz, DMSO-d6) δ ppm: 7.24-6.81 (m, 6H), 6.40-5.99 (m, 1H), 5.34-1.93 (m, 19H), 1.30-0.52 (m, 7H).

### Step D: tert-butyl 6-[4-(ethoxycarbonyl)-1,5-dimethylpyrrol-2-yl]-7-{[(3R)-3-methyl-3,4-dihydro-1H-isoquinolin-2-yl]carbonyl}-3,4-dihydro-1H-isoquinoline-2-carboxylate

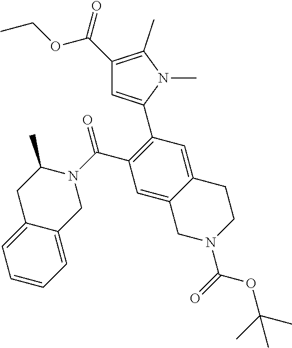 [SMILES: CCOC(=O)c1cc(-c2cc3c(cc2C(=O)N2Cc4ccccc4C[C@H]2C)CN(C(=O)OC(C)(C)C)CC3)n(C)c1C]

To a solution of the product from Step C (2.94 g, 6.23 mmol, 1 eq.) in THE (45 mL) and water (6 mL) was added bis(tert-butyl) dicarbonate (1.43 g, 6.55 mmol, 1.05 eq.) followed by TEA (1.73 mL, 12.47 mmol, 2 eq.) and the mixture stirred at rt for 12 h. The solvents were removed in vacuo and the residue partitioned between EtOAc and sat aq. NH₄Cl solution. The phases were separated, and the organic phase was washed with sat. aq. NaCl solution, dried over MgSO₄ and concentrated in vacuo to afford the title product (3.39 g, 5.93 mmol, 95%).

HRMS calcd for C₃₄H₄₁N₃O₅: 571.305, found 572.315 [M+H]⁺

¹H NMR (400 MHz, DMSO-d6) δ ppm: 7.33-6.79 (m, 6H), 6.41-6.00 (m, 1H), 5.34-1.99 (m, 19H), 1.51-1.37 (m, 9H), 1.28-0.52 (m, 6H).

### Step E: 5-[2-(tert-butoxycarbonyl)-7-{[(3R)-3-methyl-3,4-dihydro-1H-isoquinolin-2-yl]carbonyl}-3,4-dihydro-1H-isoquinolin-6-yl]-1,2-dimethylpyrrole-3-carboxylic acid

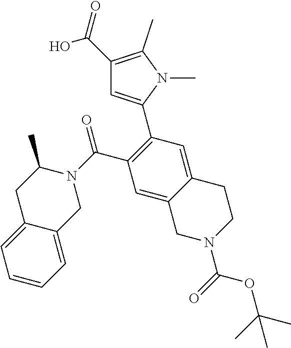 [SMILES: Cc1c(C(=O)O)cc(-c2cc3c(cc2C(=O)N2Cc4ccccc4C[C@H]2C)CN(C(=O)OC(C)(C)C)CC3)n1C]

LiOH·H₂O (995 mg, 23.72 mmol, 4 eq.) was added to a solution of the product from Step D (3.39 g, 5.93 mmol, 1 eq.) in a mixture of MeOH (40 mL) and water (20 mL) and heated at 100° C. for 24 h. The mixture was allowed to cool to rt and MeOH removed in vacuo. The aqueous residue was acidified with 1 M aq. HCl solution to pH 5, diluted with water (10 mL) and extracted with DCM. The combined organic extracts were dried over MgSO₄ and concentrated in vacuo. Purification by automated flash chromatography eluting with a gradient of 0-6% MeOH in DCM afforded the title product (3.1 g, 5.7 mmol, 96%).

HRMS calcd for C₃₂H₃₇N₃O₅: 543.273, found 544.283 [M+H]⁺

¹H NMR (400 MHz, DMSO-d6) δ ppm: 11.53 (s, 1H), 7.32-6.80 (m, 6H), 6.38-6.06 (m, 1H), 5.37-1.89 (m, 17H), 1.50-1.38 (m, 9H), 1.08-0.50 (m, 3H).

### Preparation IIb: 5-[2-(tert-butoxycarbonyl)-7-{[(3S)-3-(piperidin-1-ylmethyl)-3,4-dihydro-1H-isoquinolin-2-yl]carbonyl}-3,4-dihydro-1H-isoquinolin-6-yl]-1,2-dimethylpyrrole-3-carboxylic acid

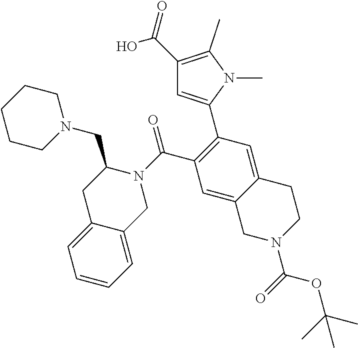 [SMILES: Cc1c(C(=O)O)cc(-c2cc3c(cc2C(=O)N2Cc4ccccc4C[C@H]2CN2CCCCC2)CN(C(=O)OC(C)(C)C)CC3)n1C]

### Step A: (3S)-3-[(piperidin-1-yl)carbonyl]-3,4-dihydro-1H-isoquinoline-2-carboxylate

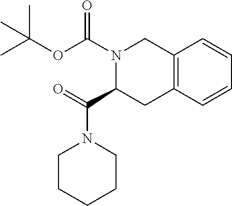 [SMILES: CC(C)(C)OC(=O)N1Cc2ccccc2C[C@H]1C(=O)N1CCCCC1]

Using General procedure 5 and Boc-(3S)-1,2,3,4-tetrahydroisoquinoline-3-carboxylic acid (5.56 g, 20.05 mmol, 1 eq.) and piperidine (1.99 mL, 20.05 mmol, 1 eq.) as the appropriate amine afforded the title product (6.11 g, 17.74 mmol, 88%).

LRMS calcd for C₂₀H₂₈N₂O₃: 344.2, found 345.2 [M+H]⁺

¹H NMR (400 MHz, DMSO-d6) δ ppm: 7.31-7.04 (m, 4H), 5.25-4.87 (m, 1H), 4.72-4.58 (m, 1H), 4.47-4.25 (m, 1H), 3.64-3.01 (m, 5H), 2.94-2.73 (m, 1H), 1.71-1.28 (m, 15H).

### Step B: (3S)-3-(piperidin-1-ylmethyl)-1,2,3,4-tetrahydroisoquinoline dihydrochloride

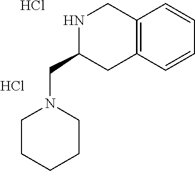 [SMILES: Cl ; c1ccc2c(c1)CN[C@H](CN1CCCCC1)C2 ; Cl]

Using General procedure 6 and the product from Step A (6.09 g, 17.69 mmol, 1 eq.) as the appropriate THIQ amide afforded the title product (5.73 g, quant.).

LRMS calcd for C₁₅H₂₂N₂: 230.2, found 231.0 [M+H]⁺

¹H NMR (400 MHz, D₂O) δ ppm: 7.43-7.24 (m, 4H), 4.54 (s, 2H), 4.32-4.21 (m, 1H), 3.78-3.54 (m, 4H), 3.36 (dd, J=17.3, 4.9 Hz, 1H), 3.20-3.02 (m, 3H), 2.09-1.73 (m, 5H), 1.63-1.44 (m, 1H).

### Step C: benzyl 6-[4-(ethoxycarbonyl)-1,5-dimethylpyrrol-2-yl]-7-{[(3S)-3-(piperidin-1-ylmethyl)-3,4-dihydro-1H-isoquinolin-2-yl]carbonyl}-3,4-dihydro-1H-isoquinoline-2-carboxylate

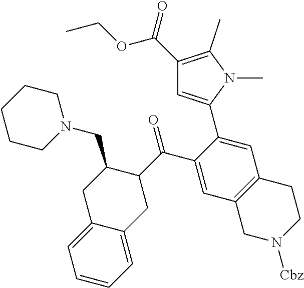 [SMILES: CCOC(=O)c1cc(-c2cc3c(cc2C(=O)C2Cc4ccccc4C[C@H]2CN2CCCCC2)CN(C(=O)OCc2ccccc2)CC3)n(C)c1C]

To a solution of Preparation I (3 g, 6.3 mmol, 1 eq.) and the product from Step B (2.1 g, 6.93 mmol, 1.1 eq.) in DMF (25 mL) was added DIPEA (4.17 mL, 25.18 mmol, 4 eq.) and PyBop (3.6 g, 6.93 mmol, 1.1 eq.) and the mixture stirred at rt for 3 h. The mixture was diluted with water (40 mL) and sat. aq. NaHCO₃ solution (10 mL) and stirred for 15 mins. The resultant cream precipitate was collected by filtration, washing with water. The filter cake was dissolved in EtOAc, washed with sat. aq. NaHCO₃ solution, dried over MgSO₄ and concentrated in vacuo to afford the title product (4.34 g, quant.).

LRMS calcd for C₄₂H₄₈N₄O₅: 688.4, found 689.6 [M+H]⁺

¹H NMR (400 MHz, DMSO-d6) δ ppm: 7.47-6.82 (m, 11H), 6.37-6.05 (m, 1H), 5.22-1.82 (m, 27H), 1.61-1.03 (m, 9H).

### Step D: ethyl 1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxylate

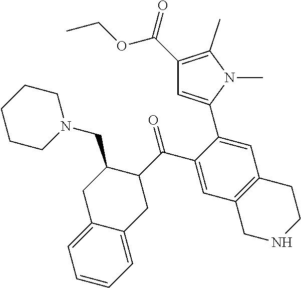 [SMILES: CCOC(=O)c1cc(-c2cc3c(cc2C(=O)C2Cc4ccccc4C[C@H]2CN2CCCCC2)CNCC3)n(C)c1C]

To a solution of the product from Step C (4.34 g, 6.3 mmol, 1 eq.) in MeOH (100 mL) was added 10% Pd/C (100 mg). The mixture was evacuated and backfilled with N₂, then evacuated and flushed with H₂ and then shaken at rt for 3 h under an atmosphere of H₂. The mixture was filtered through a celite cartridge, washing with MeOH. The solvent was removed in vacuo to afford the title product (3.69 g, quant.).

LRMS calcd for C₃₄H₄₂N₄O₃: 544.3, found 555.4 [M+H]⁺

¹H NMR (400 MHz, DMSO-d6) δ ppm: 7.22-6.81 (m, 6H), 6.36-6.01 (m, 1H), 5.14-1.65 (m, 25H), 1.57-1.05 (m, 10H).

### Step E: tert-butyl 6-[4-(ethoxycarbonyl)-1,5-dimethylpyrrol-2-yl]-7-{[(3S)-3-(piperidin-1-ylmethyl)-3,4-dihydro-1H-isoquinolin-2-yl]carbonyl}-3,4-dihydro-1H-isoquinoline-2-carboxylate

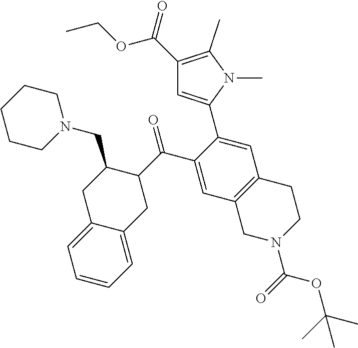 [SMILES: CCOC(=O)c1cc(-c2cc3c(cc2C(=O)C2Cc4ccccc4C[C@H]2CN2CCCCC2)CN(C(=O)OC(C)(C)C)CC3)n(C)c1C]

To a solution of the product from Step D (3.6 g, 6.49 mmol, 1 eq.) in THE (45 mL) and water (6 mL) was added bis(tert-butyl) dicarbonate (1.49 g, 6.81 mmol, 1.05 eq.) and TEA (1.8 mL, 12.98 mmol, 2 eq.) and the mixture stirred at rt for 5 h. The solvent was removed in vacuo and the residue partitioned between EtOAc and sat aq. NH₄Cl solution. Water was added, the phases were separated, and the organic phase was washed with brine, dried over MgSO₄ and concentrated in vacuo to afford the title product (4.27 g, quant.).

LRMS calcd for C₃₉H₅₀N₄O₅: 654.4, found 655.6 [M+H]⁺

¹H NMR (400 MHz, DMSO-d6) δ ppm: 7.31-6.81 (m, 6H), 6.37-6.03 (m, 1H), 5.24-1.65 (m, 25H), 1.59-1.06 (m, 18H).

### Step F. 5-[2-(tert-butoxycarbonyl)-7-{[(3S)-3-(piperidin-1-ylmethyl)-3,4-dihydro-1H-isoquinolin-2-yl]carbonyl}-3,4-dihydro-1H-isoquinolin-6-yl]-1,2-dimethylpyrrole-3-carboxylic acid

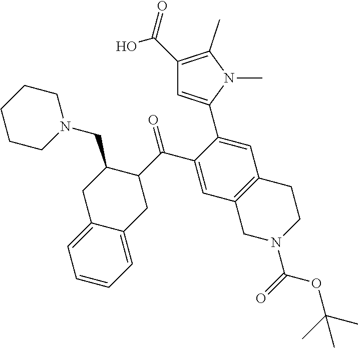 [SMILES: Cc1c(C(=O)O)cc(-c2cc3c(cc2C(=O)C2Cc4ccccc4C[C@H]2CN2CCCCC2)CN(C(=O)OC(C)(C)C)CC3)n1C]

To a heterogenous mixture of the product from Step E (4.27 g, 6.52 mmol, 1 eq.) in MeOH (40 mL) and water (10 mL) was added LiOH H₂O (1.09 g, 26.08 mmol, 4 eq.) and the mixture was refluxed at 100° C. for 24 h. The mixture was allowed to cool to rt and MeOH removed in vacuo. The aqueous residue was neutralised with 2 M aq. HCl solution and extracted with DCM. The combined organic extracts were dried over MgSO₄ and concentrated in vacuo. Purification by automated flash chromatography eluting with a gradient of 0-14% MeOH (1% NH₃) in DCM afforded the title product (2.56 g, 4.09 mmol, 63%).

LRMS calcd for (C₃₇H₄₆N₄O₅): 626.3, found 627.6 [M+H]⁺

¹H NMR (400 MHz, DMSO-d6) δ ppm: 11.29 (br s, 1H), 7.32-6.79 (m, 6H), 6.38-6.07 (m, 1H), 5.14-1.68 (m, 23H), 1.57-1.19 (m, 15H).

Preparation IIc: 5-[2-(tert-butoxycarbonyl)-7-{[(3S)-3-(pyrrolidin-1-ylmethyl)-3,4-dihydro-1H-isoquinolin-2-yl]carbonyl}-3,4-dihydro-1H-isoquinolin-6-yl]-1,2-dimethylpyrrole-3-carboxylic acid

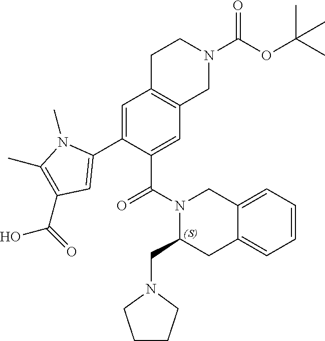

### Step A: tert-butyl (3S)-3-[(pyrrolidin-1-yl)carbonyl]-3,4-dihydro-1H-isoquinoline-2-carboxylate

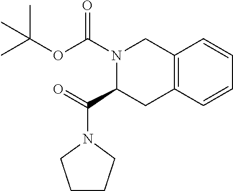 [SMILES: CC(C)(C)OC(=O)N1Cc2ccccc2C[C@H]1C(=O)N1CCCC1]

Using General procedure 5 and Boc-(3S)-1,2,3,4-tetrahydroisoquinoline-3-carboxylic acid (4 g, 14.42 mmol, 1 eq.) and pyrrolidine (0.92 mL, 15.14 mmol, 1.05 eq.) as the appropriate amine afforded the title product (4.33 g, 13.1 mmol, 91%).

LRMS calcd for C₁₉H₂₆N₂O₃: 330.2, found 331.0 [M+H]⁺

¹H NMR (400 MHz, DMSO-d6) δ ppm: 7.29-7.08 (m, 4H), 4.96-4.18 (m, 3H), 3.60-2.76 (m, 6H), 2.01-1.67 (m, 4H), 1.51-1.24 (m, 9H).

### Step B: (3S)-3-(pyrrolidin-1-ylmethyl)-1,2,3,4-tetrahydroisoquinoline dihydrochloride

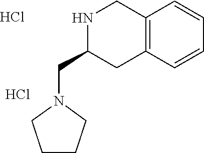 [SMILES: Cl ; Cl ; c1ccc2c(c1)CN[C@H](CN1CCCC1)C2]

Using General procedure 6 and the product from Step A (4.33 g, 13.1 mmol, 1 eq.) as the appropriate THIQ amide afforded the title product (3.79 g, 13.1 mmol, quant.).

LRMS calcd for C₁₄H₂₀N₂: 216.2, found 217.0 [M+H]⁺

¹H NMR (400 MHz, DMSO-d6) δ ppm: 11.03-10.76 (br m, 1H), 10.26-9.84 (br m, 2H), 7.35-7.16 (m, 4H), 4.50-4.35 (m, 2H), 4.13-4.00 (m, 1H), 3.91-2.95 (m, 8H), 2.14-1.80 (m, 4H).

### Step C: 5-[2-(tert-butoxycarbonyl)-7-{[(3S)-3-(pyrrolidin-1-ylmethyl)-3,4-dihydro-1H-isoquinolin-2-yl]carbonyl}-3,4-dihydro-1H-isoquinolin-6-yl]-1,2-dimethylpyrrole-3-carboxylic acid

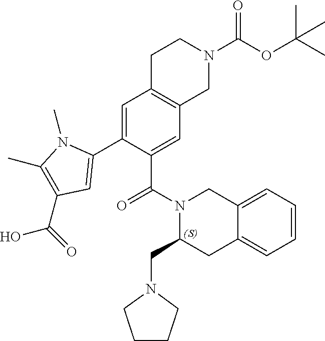

The title compound was prepared in an analogous fashion to Preparation IIb using the product from Step B in the next step.

LRMS calcd for C₃₆H₄₄N₄O₅: 612.3, found 613.6 [M+H]⁺

¹H NMR (400 MHz, DMSO-d6) δ ppm: 11.50 (br s, 1H), 7.30-6.79 (m, 6H), 6.38-6.08 (m, 1H), 5.24-1.27 (m, 36H).

### Preparation IIIa: 4-[(tert-butyldimethylsilyl)oxy]aniline

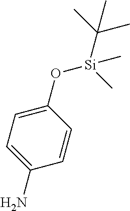

To a solution of imidazole (13.72 g, 201.6 mmol, 2.5 eq.) and 4-aminophenol (8.8 g, 80.64 mmol, 1 eq.) in MeCN (150 mL), cooled to 0° C. under N₂, was added TBDMSCl (12.76 g, 84.67 mmol, 1.05 eq.). The mixture was allowed to warm to rt and stirred for 4 h. The mixture was partitioned between EtOAc and water. The phases were separated, and the organic phase was washed with brine, dried over MgSO₄ and concentrated in vacuo. The residue was diluted with heptane (40 mL) and left to stir for 1 h. The precipitate was removed by filtration and the filtrate concentrated in vacuo to afford the title product (19.6 g, quant.).

HRMS calcd for (C₁₂H₂₁NOSi): 223.139, found 224.162 [M+H]⁺

¹H NMR (400 MHz, DMSO-d6) δ ppm: 6.56-6.48 (m, 2H), 6.48-6.40 (m, 2H), 4.87 (br s, 2H), 0.92 (s, 9H), 0.10 (s, 6H).

### Preparation IIIb: 4-[(triisopropylsilyl)oxy]aniline

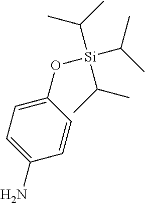 [SMILES: CC(C)[Si](Oc1ccc(N)cc1)(C(C)C)C(C)C]

The title product was prepared in accordance with the method described in Preparation IIa using 4-aminophenol (10.7 g, 98.05 mmol, 1 eq.) and TIPSCl (22.06 mL, 102.95 mmol, 1.05 eq.). The compound was obtained with a yield of 97% (25.3 g, 95.3 mmol).

LRMS calcd for (C₁₅H₂₇NOSi): 265.2, found 266.2 [M+H]⁺

¹H NMR (400 MHz, DMSO-d6) δ ppm: 6.62-6.51 (m, 2H), 6.49-6.41 (m, 2H), 4.60 (s, 2H), 1.22-1.09 (m, 3H), 1.04 (d, J=7.1 Hz, 18H).

### Preparation IVa: N-(5-cyano-1,2-dimethylpyrrol-3-yl)-N-(4-hydroxyphenyl)-1,2-dimethyl-5-(7-{[(3R)-3-methyl-3,4-dihydro-1H-isoquinolin-2-yl]carbonyl}-1,2,3,4-tetrahydroisoquinolin-6-yl)pyrrole-3-carboxamide hydrochloride

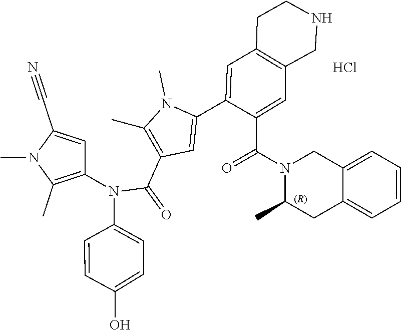

### Step A: 4-bromo-1,5-dimethylpyrrole-2-carbonitrile

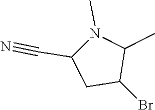

A solution of Br₂ (6.74 mL, 131.08 mmol, 1.05 eq.) in AcOH (70 mL) was added dropwise to a solution of 1,5-dimethylpyrrole-2-carbonitrile (15 g, 124.84 mmol, 1 eq.) in AcOH (250 mL), cooled to 10° C. The mixture was stirred at 10° C. for 1 h, and then allowed to warm to rt, and again stirred for 3 h. The mixture was poured into ice water (500 mL) and the resulting precipitate was collected by filtration, washed with water, and dried in vacuo to afford the title product, (23.7 g, 119.07 mmol, 95%).

¹H NMR (400 MHz, DMSO-d6) δ ppm: 7.05 (s, 1H), 3.65 (s, 3H), 2.22 (s, 3H).

### Step B: 4-({4-[(tert-butyldimethylsilyl)oxy]phenyl}amino)-1,5-dimethylpyrrole-2-carbonitrile

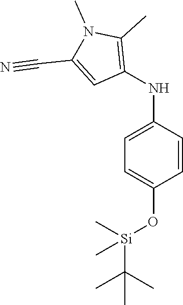 [SMILES: Cc1c(Nc2ccc(O[Si](C)(C)C(C)(C)C)cc2)cc(C#N)n1C]

Using General procedure 1a and the product from Step A (6.1 g, 30.65 mmol, 1 eq.) as the appropriate aryl bromide and Preparation IIIa (7.19 g, 32.18 mmol, 1.05 eq.) as the appropriate aniline afforded the title product (8.2 g, 24 mmol, 78%).

HRMS calcd for C₁₉H₂₇N₃OSi: 341.192, found 342.207 [M+H]⁺

¹H NMR (400 MHz, DMSO-d6) δ ppm: 6.87 (s, 1H), 6.73 (s, 1H), 6.64-6.56 (m, 2H), 6.54-6.46 (m, 2H), 3.61 (s, 3H), 2.09 (s, 3H), 0.92 (s, 9H), 0.12 (s, 6H).

### Step C: tert-butyl 6-[4-({4-[(tert-butyldimethylsilyl)oxy]phenyl}(5-cyano-1,2-dimethylpyrrol-3-yl)carbamoyl)-1,5-dimethylpyrrol-2-yl]-7-{[(3R)-3-methyl-3,4-dihydro-1H-isoquinolin-2-yl]carbonyl}-3,4-dihydro-1H-isoquinoline-2-carboxylate

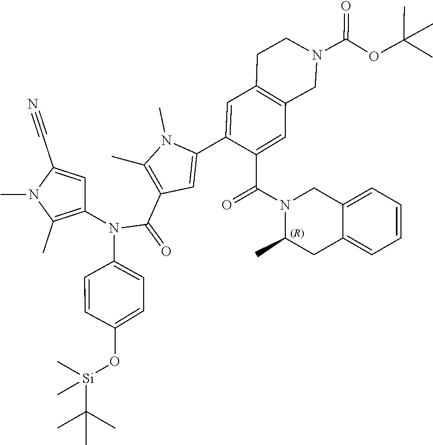

The title compound was prepared according to General procedure 2a using Preparation IIa (2.5 g, 4.6 mmol, 1 eq.) as the appropriate acid and the product from Step B (3.14 g, 9.2 mmol, 2 eq.) as the appropriate aniline. Purification by automated flash chromatography eluting with a gradient of 0-6% MeOH in DCM afforded the title product (2.49 g, 2.87 mmol, 62%).

HRMS calcd for C₅₁H₆₂N₆O₅Si: 866.455, found 867.465 [M+H]⁺

### Step D: N-(5-cyano-1,2-dimethylpyrrol-3-yl)-N-(4-hydroxyphenyl)-1,2-dimethyl-5-(7-{[(3R)-3-methyl-3,4-dihydro-1H-isoquinolin-2-yl]carbonyl}-1,2,3,4-tetrahydroisoquinolin-6-yl)pyrrole-3-carboxamide hydrochloride

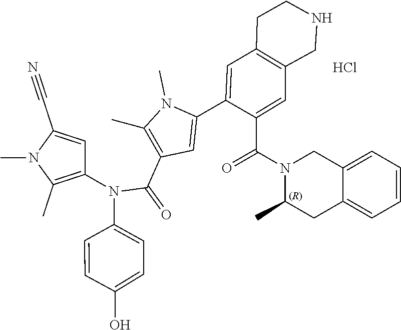

A solution of the product from Step C (2.8 g, 3.23 mmol, 1 eq.) in MeOH (5 mL) was treated with 3 M HCl in MeOH (10 mL, 30 mmol) and stirred at rt for 3 h. The solvents were removed in vacuo and then dried under high vacuum to afford the title product (2.53 g, quant.).

HRMS calcd for C₄₀H₄₀N₆O₃: 652.316, found 653.326 [M+H]⁺

### Preparation IVb: N-(5-cyano-1,2-dimethylpyrrol-3-yl)-N-(4-hydroxyphenyl)-1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxamide

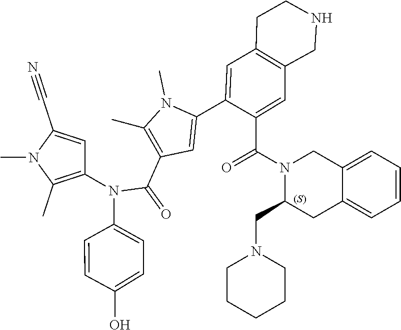

### Step A: 6-[4-({4-[(tert-butyldimethylsilyl)oxy]phenyl}(5-cyano-1,2-dimethylpyrrol-3-yl)carbamoyl)-1,5-dimethylpyrrol-2-yl]-7-{[(3S)-3-(piperidin-1-ylmethyl)-3,4-dihydro-1H-isoquinolin-2-yl]carbonyl}-3,4-dihydro-1H-isoquinoline-2-carboxylate

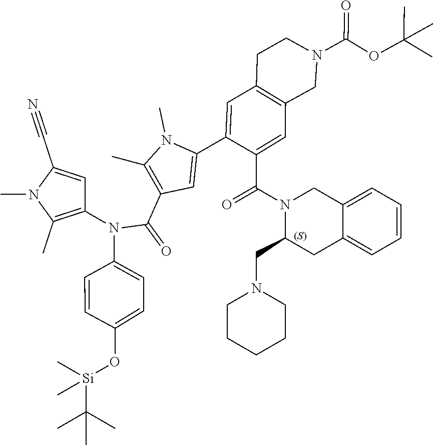

The title compound was prepared according to General procedure 2a using Preparation IIb (2.2 g, 3.51 mmol, 1 eq.) as the appropriate acid and the product from Preparation IVa, Step B (1.68 g, 4.91 mmol, 1.4 eq.) as the appropriate aniline. Purification by automated flash chromatography eluting with a gradient of 0-10% MeOH (1% NH₃) in DCM afforded the title product (2.29 g, 2.4 mmol, 69%).

LRMS calcd for C₅₆H₇₁N₇O₅Si: 949.5, found 950.6 [M+H]⁺

### Step B: N-(5-cyano-1,2-dimethylpyrrol-3-yl)-N-(4-hydroxyphenyl)-1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxamide

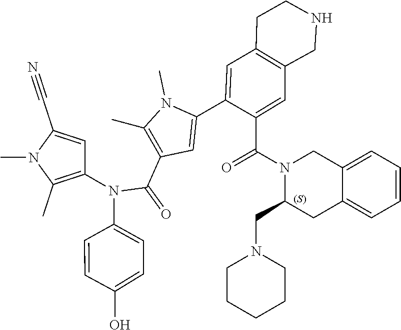

A solution of the product from Step A (2.3 g, 2.42 mmol, 1 eq.) in MeOH (10 mL) was treated with 3 M HCl in MeOH (7 mL, 21 mmol) and stirred at rt for 12 h. The solvents were removed in vacuo and the residue was dissolved in 1:1 DCM/MeOH followed by the addition of TEA (1.5 mL, 3 eq.) and then purified by reverse phase automated flash chromatography eluting with a gradient of 20-100% MeCN in water. The product was lyophilised to afford the title product (1.18 g, 1.6 mmol, 66%).

LRMS calcd for (C₄₅H₄₉N₇O₃): 735.4, found 736.6 [M+H]⁺

¹H NMR (400 MHz, DMSO-d6) δ ppm: 9.42-9.27 (m, 1H), 7.25-6.39 (m, 11H), 5.46-5.09 (m, 1H), 5.09-1.66 (m, 27H), 1.60-1.20 (m, 6H).

### Preparation IVc: N-(5-cyano-1,2-dimethylpyrrol-3-yl)-N-(4-hydroxyphenyl)-1,2-dimethyl-5-(7-{[(3S)-3-(pyrrolidin-1-ylmethyl)-3,4-dihydro-1H-isoquinolin-2-yl]carbonyl}-1,2,3,4-tetrahydroisoquinolin-6-yl)pyrrole-3-carboxamide

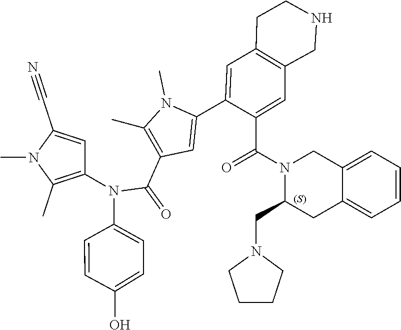

### Step A: tert-butyl 6-[4-({4-[(tert-butyldimethylsilyl)oxy]phenyl}(5-cyano-1,2-dimethylpyrrol-3-yl)carbamoyl)-1,5-dimethylpyrrol-2-yl]-7-{[(3S)-3-(pyrrolidin-1-ylmethyl)-3,4-dihydro-1H-isoquinolin-2-yl]carbonyl}-3,4-dihydro-1H-isoquinoline-2-carboxylate

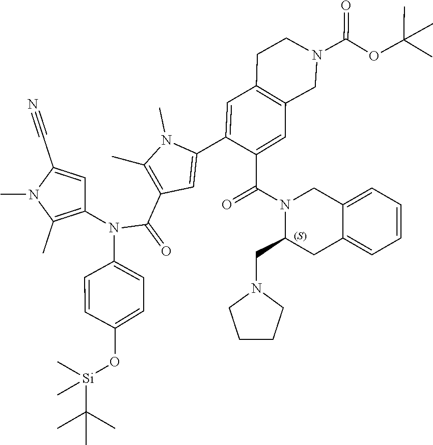

The title compound was prepared according to General procedure 2a using Preparation IIc (1.6 g, 2.61 mmol, 1 eq.) as the appropriate acid and the product from Preparation IVa, Step B (1.34 g, 3.92 mmol, 1.5 eq.) as the appropriate aniline. Purification by automated flash chromatography eluting with a gradient of 0-8% MeOH in DCM afforded the title product (1.9 g, 2.03 mmol, 78%).

LRMS calcd for C₅₅H₆₉N₇O₅Si: 935.5, found 936.6 [M+H]⁺

### Step B: N-(5-cyano-1,2-dimethylpyrrol-3-yl)-N-(4-hydroxyphenyl)-1,2-dimethyl-5-(7-{[(3S)-3-(pyrrolidin-1-ylmethyl)-3,4-dihydro-1H-isoquinolin-2-yl]carbonyl}-1,2,3,4-tetrahydroisoquinolin-6-yl)pyrrole-3-carboxamide

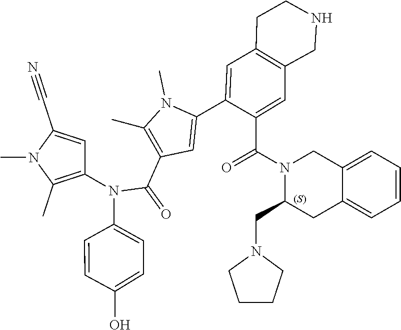

A solution of the product from Step A 1.9 g, 2.03 mmol, 1 eq.) in MeOH (19 mL) was treated with 3 M HCl in MeOH (2.28 mL, 6.83 mmol) and stirred at rt for 12 h. The mixture was diluted with DCM and basified by the addition of sat. aq. NaHCO₃ solution. The layers were separated, and the aqueous phase was extracted twice more with DCM. The combined organic extracts were dried over MgSO₄ and concentrated under reduced pressure. Purification by reverse phase automated flash chromatography at pH 4, eluting with a gradient of 20-100% MeCN in water afforded the title product (1.05 g, 1.45 mmol, 71%).

LRMS calcd for C₄₄H₄₇N₇O₃: 721.4, found 722.6 [M+H]⁺

¹H NMR (400 MHz, DMSO-d6) δ ppm: 9.41-9.28 (m, 1H), 7.26-6.42 (m, 11H), 5.45-5.11 (m, 1H), 5.03-1.91 (m, 29H), 1.86-1.47 (m, 4H).

### Preparation Va: {4-[2-(morpholin-4-yl)ethoxy]phenyl}acetic acid

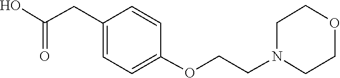 [SMILES: O=C(O)Cc1ccc(OCCN2CCOCC2)cc1]

### Step A: methyl 2-{4-[2-(morpholin-4-yl)ethoxy]phenyl}acetate

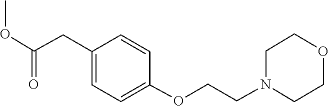 [SMILES: COC(=O)Cc1ccc(OCCN2CCOCC2)cc1]

Using General procedure 7a and methyl 4-hydroxyphenylacetate (2.5 g, 15.04 mmol, 1 eq.) as the appropriate phenol and 4-(2-chloroethyl)morpholine hydrochloride (4.48 g, 24.07 mmol, 1.6 eq.) as the appropriate alkyl halide afforded the title product (3.24 g, 11.59 mmol, 77%).

LRMS calcd for (C₁₅H₂₁NO₄): 279.2, found 280.2 [M+H]⁺

¹H NMR (400 MHz, DMSO-d6) δ ppm: 7.20-7.12 (m, 2H), 6.93-6.85 (m, 2H), 4.06 (t, J=5.8 Hz, 2H), 3.60 (s, 3H), 3.59 (s, 2H), 3.59-3.56 (m, 4H), 2.68 (t, J=5.8 Hz, 2H), 2.50-2.43 (m, 4H).

### Step B: {4-[2-(morpholin-4-yl)ethoxy]phenyl}acetic acid

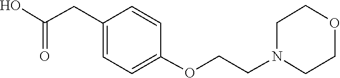 [SMILES: O=C(O)Cc1ccc(OCCN2CCOCC2)cc1]

The product from Step A (3.26 g, 11.67 mmol, 1 eq.) was hydrolysed using General procedure 8 to afford the title product (3.1 g, 11.63 mmol, quant).

LRMS calcd for (C₁₄H₁₉NO₄): 265.1, found 266.2 [M+H]⁺

¹H NMR (400 MHz, DMSO-d6) δ ppm: 7.15-7.09 (m, 2H), 6.87-6.79 (m, 2H), 4.04 (t, J=5.8 Hz, 2H), 3.60-3.54 (m, 4H), 3.34 (s, 2H), 2.66 (t, J=5.8 Hz, 2H), 2.49-2.42 (m, 4H).

### Preparation Vb: {2-fluoro-4-[2-(morpholin-4-yl)ethoxy]phenyl}acetic acid

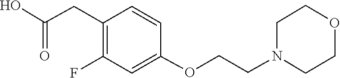 [SMILES: O=C(O)Cc1ccc(OCCN2CCOCC2)cc1F]

### Step A: methyl 2-(2-fluoro-4-hydroxyphenyl)acetate

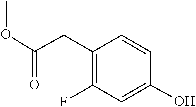

Concentrated H₂SO₄ (5 mL) was added dropwise to MeOH (100 mL), cooled to 0° C. After addition, 2-fluoro-4-hydroxyphenylacetic acid (5 g, 29.39 mmol, 1 eq.) was added in portions and then the mixture slowly heated to reflux where it was held for 3 h. The mixture was allowed to cool to rt and poured onto a mixture of ice/water (200 mL) with stirring. The mixture was extracted with DCM and the combined organic extracts were washed with brine, dried over MgSO₄ and concentrated in vacuo to afford the title product (6.1 g, 29.15 mmol, 99%).

¹H NMR (400 MHz, DMSO-d6) δ ppm: 9.80 (s, 1H), 7.10 (t, J=8.8 Hz, 1H), 6.60-6.50 (m, 2H), 3.60 (s, 3H), 3.57 (s, 2H).

### Step B: methyl 2-{2-fluoro-4-[2-(morpholin-4-yl)ethoxy]phenyl}acetate

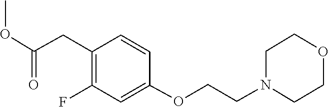 [SMILES: COC(=O)Cc1ccc(OCCN2CCOCC2)cc1F]

Using General procedure 7a and the product from Step A (4.22 g, 22.91 mmol, 1 eq.) as the appropriate phenol and 4-(2-chloroethyl)morpholine hydrochloride (8.53 g, 45.83 mmol, 2 eq.) as the appropriate alkyl halide afforded the title product (4.99 g, 16.78 mmol, 73%).

HRMS calcd for (C₁₅H₂₀NO₄F): 297.138, found 298.154 [M+H]⁺

¹H NMR (400 MHz, DMSO-d6) δ ppm: 7.22 (t, J=8.7 Hz, 1H), 6.83 (dd, J=12.0, 2.5 Hz, 1H), 6.78-6.71 (m, 1H), 4.08 (t, J=5.7 Hz, 2H), 3.63 (d, J=1.3 Hz, 2H), 3.61 (s, 3H), 3.59-3.55 (m, 4H), 2.67 (t, J=5.7 Hz, 2H), 2.49-2.42 (m, 4H).

### Step C: [2-fluoro-4-[2-(morpholin-4-yl)ethoxy]phenyl}acetic acid

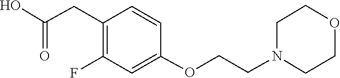 [SMILES: O=C(O)Cc1ccc(OCCN2CCOCC2)cc1F]

The product from Step B (4.99 g, 16.78 mmol) was hydrolysed using General procedure 8 to afford the title product (4.65 g, 16.41 mmol, 99%).

LRMS calcd for (C₁₄H₁₈NO₄F): 283.1, found 284.0 [M+H]⁺

¹H NMR (400 MHz, DMSO-d6) δ ppm: 7.17 (t, J=8.7 Hz, 1H), 6.75 (dd, J=12.0, 2.5 Hz, 1H), 6.72-6.67 (m, 1H), 4.06 (t, J=5.8 Hz, 2H), 3.60-3.53 (m, 4H), 3.37 (s, 2H), 2.68-2.65 (m, 2H), 2.49-2.42 (m, 4H).

### Preparation Vc: 2-[4-(2-tetrahydropyran-4-ylethoxy)phenyl]acetic acid

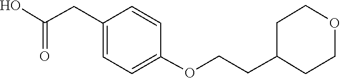 [SMILES: O=C(O)Cc1ccc(OCCC2CCOCC2)cc1]

### Step A: methyl 2-{4-[2-(oxan-4-yl)ethoxy]phenyl}acetate

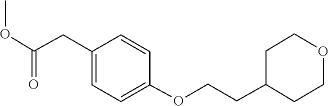 [SMILES: COC(=O)Cc1ccc(OCCC2CCOCC2)cc1]

Using General procedure 7a and methyl 4-hydroxyphenylacetate (2 g, 12.04 mmol, 1 eq.) as the appropriate phenol and 4-(2-bromoethyl)tetrahydro-2H-pyran (2.32 mL, 15.65 mmol, 1.3 eq.) as the appropriate alkyl halide afforded the title product (2.95 g, 10.61 mmol, 88%).

¹H NMR (400 MHz, DMSO-d6) δ ppm: 7.19-7.11 (m, 2H), 6.91-6.83 (m, 2H), 3.98 (t, J=6.3 Hz, 2H), 3.87-3.78 (m, 2H), 3.59 (s, 3H), 3.58 (s, 2H), 3.28 (td, J=11.8, 2.1 Hz, 2H), 1.77-1.56 (m, 5H), 1.29-1.14 (m, 2H).

### Step B: methyl 2-{4-[2-(oxan-4-yl)ethoxy]phenyl}acetate

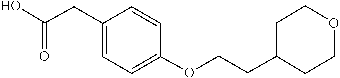 [SMILES: O=C(O)Cc1ccc(OCCC2CCOCC2)cc1]

The product from Step A (2.95 g, 10.61 mmol, 1 eq.) was hydrolysed using General procedure 8. Methanol was removed in vacuo and the aqueous residue was acidified with 2 M aq. HCl solution and extracted with DCM. The organic phase was dried over MgSO₄ and concentrated in vacuo to afford the title product (2.71 g, 10.27 mmol, 97%).

LRMS calcd for C₁₅H₂₀O₄: 264.1, found 263.2 [M−H]⁻

¹H NMR (400 MHz, DMSO-d6) δ ppm: 12.21 (s, 1H), 7.18-7.10 (m, 2H), 6.90-6.82 (m, 2H), 3.98 (t, J=6.4 Hz, 2H), 3.87-3.78 (m, 2H), 3.47 (s, 2H), 3.28 (td, J=11.8, 2.1 Hz, 2H), 1.77-1.56 (m, 5H), 1.29-1.12 (m, 2H).

### Preparation VIa: 1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxylic acid

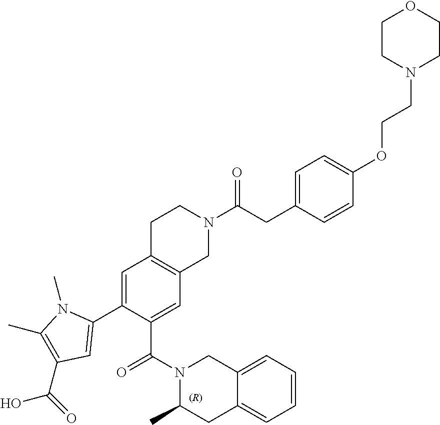

### Step A: 1,2-dimethyl-5-(7-{[(3R)-3-methyl-3,4-dihydro-1H-isoquinolin-2-yl]carbonyl}-1,2,3,4-tetrahydroisoquinolin-6-yl)pyrrole-3-carboxylic acid hydrochloride

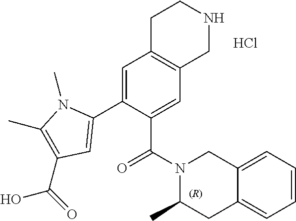

A solution of Preparation Ha (3 g, 5.52 mmol, 1 eq.) in MeOH (20 mL) was treated with 3 M HCl in MeOH (20 mL, 60 mmol) and stirred at rt for 3 h. The solvents were removed in vacuo and dried under vacuum to afford the title product (2.71 g, quant.).

LRMS calcd for C₂₇H₂₉N₃O₃: 443.2, found 444.4 [M+H]⁺

¹H NMR (400 MHz, DMSO-d6) δ ppm: 9.79-9.40 (br m, 2H), 7.42-6.79 (m, 6H), 6.43-6.04 (m, 1H), 5.37-1.87 (m, 17H), 1.14-0.46 (m, 3H).

### Step B: 1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxylic acid

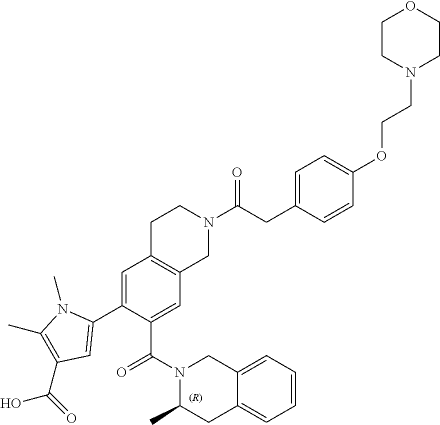

To a solution of Preparation Va (1 g, 3.77 mmol, 1 eq) in anhydrous DCM (10 mL) under N₂, was slowly added 2 M oxalyl chloride solution in DCM (0.43 mL, 4.52 mmol, 1.2 eq.) followed by DMF (1 drop), and the mixture stirred for 1 h. The solvents were removed in vacuo, the acid chloride intermediate was dissolved in anhydrous DCM (7 mL) and added dropwise to a stirred solution of the product from Step A (1.63 g, 3.39 mmol, 0.9 eq.) and DIPEA (2.5 mL, 15.08 mmol, 4 eq.) in anhydrous DCM (10 mL) under N₂. The mixture was stirred at rt for 2 h, then quenched with MeOH (5 mL) and concentrated in vacuo. Purification by automated flash chromatography eluting with a gradient of 0-8% MeOH in DCM afforded the title product (1.2 g, 1.74 mmol, 46%).

LRMS calcd for (C₄₁H₄₆N₄O₆): 690.3, found 691.6 [M+H]⁺

¹H NMR (400 MHz, DMSO-d6) δ ppm: 11.53 (s, 1H), 7.32-6.78 (m, 10H), 6.39-6.05 (m, 1H), 5.35-1.88 (m, 31H), 1.11-0.48 (m, 3H).

### Preparation VIb: 1,2-dimethyl-5-(7-{[(3R)-3-methyl-3,4-dihydro-1H-isoquinolin-2-yl]carbonyl}-2-(2-{4-[2-(oxan-4-yl)ethoxy]phenyl}acetyl)-3,4-dihydro-1H-isoquinolin-6-yl)pyrrole-3-carboxylic acid

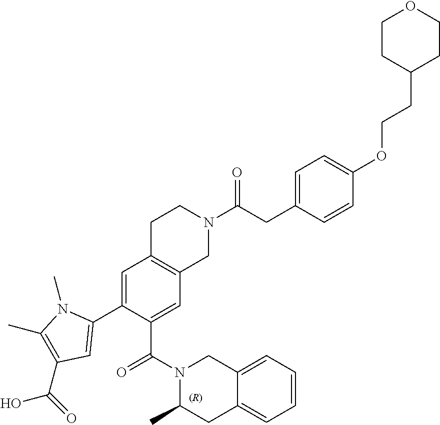

The title compound was prepared in an analogous fashion to Preparation VIa using Preparation Vc (700 mg, 2.65 mmol, 1 eq.) and the product from Preparation VIa, Step A (1.14 g, 2.38 mmol, 0.9 eq.) to afford the title product (1.04 g, 1.51 mmol, 57%).

LRMS calcd for (C₄₂H₄₇N₃O₆): 689.3, found 690.6 [M+H]⁺

¹H NMR (400 MHz, DMSO-d6) δ ppm: 11.52 (s, 1H), 7.32-6.77 (m, 10H), 6.39-6.07 (m, 1H), 5.35-1.90 (m, 25H), 1.78-1.53 (m, 5H), 1.30-1.12 (m, 2H), 1.10-0.49 (m, 3H).

### Preparation VIIa: N-[4-[tert-butyl(dimethyl)silyl]oxyphenyl]-N-(5-cyano-1,2-dimethyl-pyrrol-3-yl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

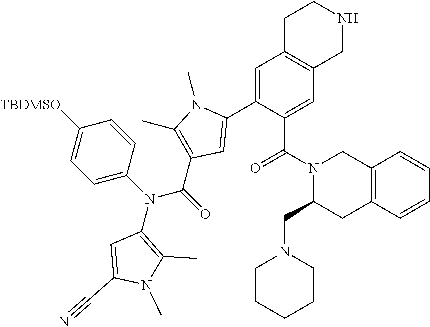 [SMILES: Cc1c(N(C(=O)c2cc(-c3cc4c(cc3C(=O)N3Cc5ccccc5C[C@H]3CN3CCCCC3)CNCC4)n(C)c2C)c2ccc(O[Si](C)(C)C(C)(C)C)cc2)cc(C#N)n1C]

### Step A: 1,2-dimethyl-5-[7-[(3S)-3-(1-piperidylmethyl)-3,4-dihydro-1H-isoquinoline-2-carbonyl]-1,2,3,4-tetrahydroisoquinolin-6-yl]pyrrole-3-carboxylic acid

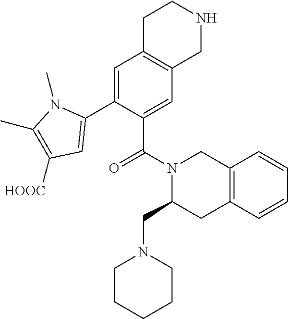 [SMILES: Cc1c(C(=O)O)cc(-c2cc3c(cc2C(=O)N2Cc4ccccc4C[C@H]2CN2CCCCC2)CNCC3)n1C]

To a solution of Preparation I (10.4 g, 21.8 mmol, 1 eq.) in DMF (60 mL) was added TBTU (7.71 g, 24.0 mmol, 1.1 eq.) followed by DIPEA (11.2 mL, 65.5 mmol, 3 eq.). After stirring for 10 mins the product from Step B of Preparation IIb (7.95 g, 26.2 mmol, 1.2 eq.) was added and the mixture was stirred at rt for 1 h then it was poured into water (600 mL) and the precipitates were filtered out, washed with water. This crude intermediate was dissolved in methanol (150 mL) and water (85 mL) then NaOH (9.18 g, 229.6 mmol, 10.5 eq.) was added and mixture was stirred at reflux temperature for 3 days. Evaporated at reduced pressure, then the pH was adjusted to 6 by the addition of cc. aq. HCl. The product was extracted with DCM/IPA=3/1 (3×100 mL). The organic phase was dried over MgSO₄ and concentrated in vacuo to afford the title product (10.97 g, 15.83 mmol, 73%).

¹H NMR (400 MHz, DMSO-d6) δ ppm 7.22-6.83 (m, 6H), 6.38-6.14 (s, 1H), 5.31-1.17 (m, 23H), 3.44-3.15 (s, 3H), 2.51-1.97 (s, 3H).

HRMS-ESI (m/z) [M+H]⁺ calcd for C₃₂H₃₉N₄O₃: 527.3017, found 527.3011

### Step B: 5-[2-(9H-fluoren-9-ylmethoxycarbonyl)-7-[(3S)-3-(1-piperidylmethyl)-3,4-dihydro-1H-isoquinoline-2-carbonyl]-3,4-dihydro-1H-isoquinolin-6-yl-1,2-dimethyl-pyrrole-3-carboxylic acid hydrochloride

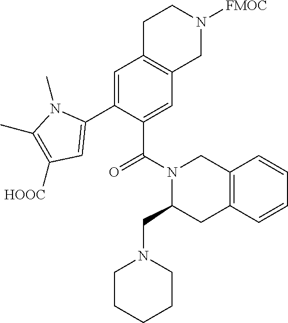

To the biphasic mixture of the product from Step A (8.63 g, 13.1 mmol, 1 eq.) dissolved in dioxane (75 mL) and NaHCO₃ (2.53 g, 30.1 mmol, 2.3 eq.) dissolved in water (75 mL) 9H-fluoren-9-ylmethyl carbonochloridate (3.73 g, 14.4 mmol, 1.1 eq.) was added dropwise and the mixture was stirred at rt for 24 h. To the reaction mixture 2M aq. HCl (131 mL; 262 mmol, 10 eq.) was added dropwise then after stirring for 10 min it was extracted with DCM. The phases were separated, and the organic phase was washed with sat. aq. NaCl solution, dried over MgSO₄ and concentrated in vacuo. The crude material was purified by automated flash chromatography using DCM and MeOH as eluents (1% NH₃) to afford the title product (7.33 g, 9.3 mmol, 71%).

¹H NMR (400 MHz, DMSO-d6) δ ppm 11.53 (br., 1H), 8.05-6.77 (m, 14H), 6.38-6.05 (br., 1H), 5.34 (br., 1H), 4.71-3.37 (br., 12H), 4.57-4.28 (br., 2H), 4.33 (t, 1H), 3.11 (br., 3H), 2.89-2.69 (br., 2H), 2.76/2.44 (br+d, 2H), 2.06-1.14 (br., 6H), 1.93 (s, 3H); ¹³C NMR (100 MHz, DMSO-d6) δ ppm 67.2, 47.3, 42.4, 31.8, 30, 28.2, 11.1.

HRMS-ESI (m/z) [M+H]⁺ calcd for C₄₇H₄₉N₄O₅: 749.3697, found 749.3702

### Step C: 9H-fluoren-9-ylmethyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-7-[(3S)-3-(1-piperidylmethyl)-3,4-dihydro-1H-isoquinoline-2-carbonyl]-3,4-dihydro-1H-isoquinoline-2-carboxylate

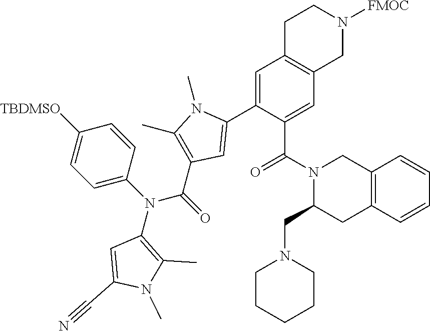

Using General procedure 2a and the product from Step B (5.33 g, 7.12 mmol, 1 eq.) as the appropriate acid, oxalyl dichloride (1.02 mL, 12.1 mmol, 1.7 eq.) and the product from Step B of Preparation IVa (2.92 g, 8.54 mmol, 1.2 eq.) as the appropriate aniline afforded the title product (4.67 g, 3.92 mmol, 55%).

¹H NMR (400 MHz, DMSO-d6) δ ppm 10.31 (brs, 1H), 8.06-6.4 (m, 19H), 5.5-1.1 (m, 26H), 5.14-4.9 (s, 1H), 3.7-2.98 (s, 6H), 2.44-1.72 (s, 6H), 0.88-0.76 (s, 9H), 0.11-0.04 (s, 6H).

HRMS-ESI (m/z) [M+H]⁺ calcd for C₆₆H₇₄N₇O₅Si: 1072.5515, found 1072.5531

### Step D: N-[4-[tert-butyl(dimethyl)silyl]oxyphenyl]-N-(5-cyano-1,2-dimethyl-pyrrol-3-yl)-1,2-dimethyl-5-[7-[(3S)-3-(1-piperidylmethyl)-3,4-dihydro-1H-isoquinoline-2-carbonyl]-1,2,3,4-tetrahydroisoquinolin-6-yl]pyrrole-3-carboxamide

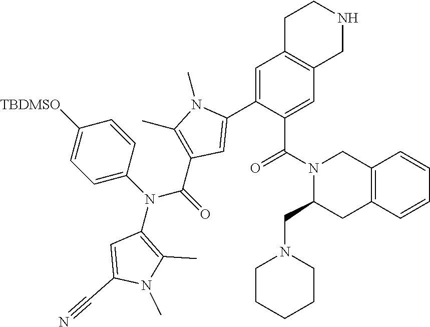 [SMILES: Cc1c(N(C(=O)c2cc(-c3cc4c(cc3C(=O)N3Cc5ccccc5C[C@H]3CN3CCCCC3)CNCC4)n(C)c2C)c2ccc(O[Si](C)(C)C(C)(C)C)cc2)cc(C#N)n1C]

To a solution of the product from Step C (1.84 g, 1.55 mmol, 1 eq.) in DCM (8 mL) morpholine (7.76 mL, 88.2 mmol, 57 eq.) was added then the mixture was stirred at rt for 18 h. The reaction mixture was concentrated in vacuo and the residue was partitioned between DCM and water. The phases were separated, and the organic phase was washed with sat. aq. NaCl solution, dried over MgSO₄ and concentrated in vacuo. The crude material was purified by automated flash chromatography using DCM and MeOH as eluents (1% NH₃) affording the title product as a yellow solid (0.762 g, 0.867 mmol, 56%).

¹H NMR (400 MHz, DMSO-d6) δ ppm 7.22-6.46 (m, 11H), 5.41-5.02 (s, 1H), 5.05-1.25 (m, 22H), 3.6-3.06 (s, 6H), 2.4-1.76 (s, 6H), 0.91-0.84 (s, 9H), 0.13-0.08 (s, 6H).

HRMS-ESI (m/z) [M+H]⁺ calcd for C₅₁H₆₄N₇O₃Si: 850.4834, found 850.4838

### Preparation VIIb: N-[4-[tert-butyl(dimethyl)silyl]oxyphenyl]-N-(5-cyano-1,2-dimethyl-pyrrol-3-yl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

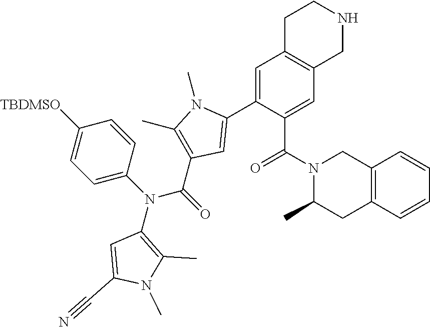 [SMILES: Cc1c(N(C(=O)c2cc(-c3cc4c(cc3C(=O)N3Cc5ccccc5C[C@H]3C)CNCC4)n(C)c2C)c2ccc(O[Si](C)(C)C(C)(C)C)cc2)cc(C#N)n1C]

### Step A: 1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxylic acid

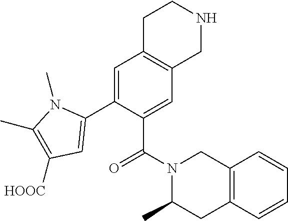 [SMILES: Cc1c(C(=O)O)cc(-c2cc3c(cc2C(=O)N2Cc4ccccc4C[C@H]2C)CNCC3)n1C]

To a solution of Preparation I (45.9 g, 96.3 mmol, 1 eq.) in DMF (240 mL) was added TBTU (34.1 g, 106 mmol, 1.1 eq.) followed by TEA (40.5 mL, 291 mmol, 3 eq.). After stirring for 10 mins the product from Step A of Preparation Ha (18.6 g, 101 mmol, 1.05 eq.) was added and the mixture was stirred at rt for 1 h then it was poured into water (1500 mL) and the precipitates were filtered out, washed with water. This crude intermediate was dissolved in methanol (250 mL) and water (25 mL) then NaOH (28 g, 700 mmol, 7.3 eq.) was added and mixture was stirred at reflux temperature for 18 h. Evaporated at reduced pressure, then the pH was adjusted to 6 by the addition of cc. aq. HCl. The product was extracted with DCM/IPA=3/1 (3×400 mL). The organic phase was dried over MgSO₄ and concentrated in vacuo. The residue was triturated in diethyl ether/acetonitrile. The formed yellow powder was filtered out then dried in vacuo to afford the title product (38.51 g, 86.8 mmol, 90%).

¹H NMR (500 MHz, DMSO-d6) δ ppm 7.35-6.8 (m, 6H), 6.35/6.28/6.13 (s/s/s, 1H), 5.34-3.23 (m, 6H), 5.01/4.88/3.78 (m/m/m, 1H), 3.46/3.43/3.38/3.15 (s/s/s/s, 3H), 3.12-2.98 (m, 2H), 3.02-2.05 (m, 2H), 2.49/2.41/1.96 (s/s/s, 3H), 1.03/0.85/0.74/0.56 (d/d/d/d, 3H); ¹³C NMR (125 MHz, DMSO-d6) δ ppm 168.9/168.4/168.2, 166.5/166.3/166.2, 111.3/111.2/110.8, 48.3/48.1/43/42.2, 34.4/34.2/34/32.6, 32.2/32.1/31.9, 25.4, 18.6/16.8/16.4, 11.8/11.7/11.2.

HRMS-ESI (m/z) [M+H]⁺ calcd for C₂₇H₃₀N₃O₃: 444.2282, found 444.2267

### Step B: 5-[2-(9H-fluoren-9-ylmethoxycarbonyl)-7-[(3R)-3-methyl-3,4-dihydro-1H-isoquinoline-2-carbonyl]-3,4-dihydro-1H-isoquinolin-6-yl]-1,2-dimethyl-pyrrole-3-carboxylic acid

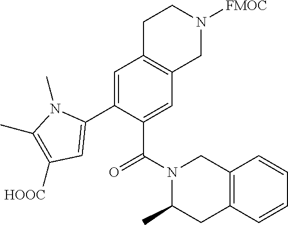

To the biphasic mixture of the product from Step A (13.96 g, 28.33 mmol, 1 eq.) dissolved in dioxane (160 mL) and NaHCO₃ (5.47 g, 65.2 mmol, 2.3 eq.) dissolved in water (160 mL) 9H-fluoren-9-ylmethyl carbonochloridate (8.06 g, 31.2 mmol, 1.1 eq.) was added dropwise and the mixture was stirred at rt for 24 h. To the reaction mixture 2M aq. HCl (42.5 mL; 85 mmol, 3 eq.) was added dropwise then after stirring for 10 min it was extracted with DCM. The phases were separated, and the organic phase was washed with sat. aq. NaCl solution, dried over MgSO₄ and concentrated in vacuo. The crude material was purified by automated flash chromatography using DCM and EtOAc as eluents affording the title product (14.77 g, 22.2 mmol, 78%).

¹H NMR (500 MHz, DMSO-d6) δ ppm 11.18 (br., 1H), 7.95-7.26 (br., 8H), 7.26-6.8 (br., 6H), 6.52-6.06 (br., 1H), 5.09-3.74 (br., 3H), 4.54 (br., 2H), 4.47 (d, 2H), 4.31 (t, 1H), 3.59 (br., 2H), 3.5-3.19 (br., 3H), 2.8 (t, 2H), 2.55-1.93 (br., 3H), 2.45 (br., 2H), 1.2-0.41 (br., 3H); ¹³C NMR (125 MHz, DMSO-d6) δ ppm 111.1, 67.2, 47.5, 45.7, 41.6, 34.6, 32, 28.3, 16.8, 11.6.

HRMS-ESI (m/z) [M+H]⁺ calcd for C₄₂H₄₀N₃O₅: 666.2962, found 666.2961

### Step C: 9H-fluoren-9-ylmethyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-7-[(3R)-3-methyl-3,4-dihydro-1H-isoquinoline-2-carbonyl]-3,4-dihydro-1H-isoquinoline-2-carboxylate

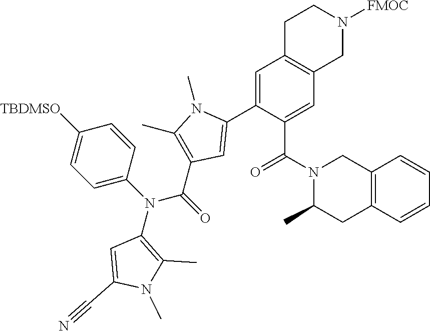

Using General procedure 2b and the product from Step B (10.67 g, 16.03 mmol, 1 eq.) as the appropriate acid, oxalyl dichloride (2.17 mL, 25.6 mmol, 1.6 eq.) and the product from Step B of Preparation IVa (6.57 g, 19.2 mmol, 1.2 eq.) as the appropriate aniline afforded the title product (8.12 g, 8.21 mmol, 51%).

¹H NMR (500 MHz, DMSO-d6) δ ppm 8.05-6.43 (m, 19H), 5.7-4.95 (s, 1H), 5.12-2.26 (m, 14H), 3.71-3 (s, 6H), 2.48-1.75 (s, 6H), 1.13-0.44 (d, 3H), 0.92-0.77 (s, 9H), 0.15-0.05 (s, 6H).

HRMS-ESI (m/z) [M+H]⁺ calcd for C₆₁H₆₅N₆O₅Si: 989.4780, found 989.4779

### Step D: N-[4-[tert-butyl(dimethyl)silyl]oxyphenyl]-N-(5-cyano-1,2-dimethyl-pyrrol-3-yl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

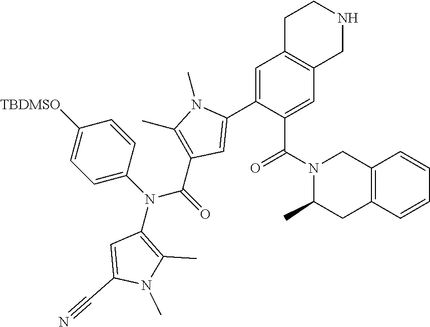 [SMILES: Cc1c(N(C(=O)c2cc(-c3cc4c(cc3C(=O)N3Cc5ccccc5C[C@H]3C)CNCC4)n(C)c2C)c2ccc(O[Si](C)(C)C(C)(C)C)cc2)cc(C#N)n1C]

To a solution of the product from Step C (8.12 g, 8.21 mmol, 1 eq.) in DCM (41 mL) morpholine (41 mL, 475 mmol, 58 eq.) was added then the mixture was stirred at rt for 18 h. The reaction mixture was concentrated in vacuo and the residue was partitioned between DCM and water. The phases were separated, and the organic phase was washed with sat. aq. NaCl solution, dried over MgSO₄ and concentrated in vacuo. The crude material was purified by automated flash chromatography using DCM and MeOH as eluents to afford the title product (4.51 g, 5.88 mmol, 72%).

¹H NMR (500 MHz, DMSO-d6) δ ppm 7.36-6.35 (m, 11H), 5.55-4.91 (s, 1H), 5.37-1.97 (m, 12H), 3.72-2.96 (s, 6H), 2.48-1.75 (s, 6H), 1.16-0.39 (d, 3H), 1-0.76 (s, 9H), 0.17-0 (s, 6H).

HRMS-ESI (m/z) [M+H]⁺ calcd for C₄₆H₅₅N₆O₃Si: 767.4099, found 767.4103

### Preparation VIII: 1,2-dimethyl-5-[7-[(3R)-3-methyl-3,4-dihydro-1H-isoquinoline-2-carbonyl]-2-(2-phenylacetyl)-3,4-dihydro-1H-isoquinolin-6-yl]pyrrole-3-carboxylic acid

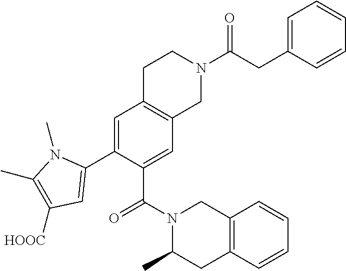 [SMILES: Cc1c(C(=O)O)cc(-c2cc3c(cc2C(=O)N2Cc4ccccc4C[C@H]2C)CN(C(=O)Cc2ccccc2)CC3)n1C]

To the ice-cooled solution of the product from Step A of Preparation VIIb (3.90 g, 8.80 mmol, 1 eq.) in water (13 mL) and NaOH (1.10 g, 26 mmol, 3 eq.) was added dropwise the solution of 2-phenylacetyl chloride (1.40 mL, 10.6 mmol, 1.2 eq.) in DCE (2.6 mL). The reaction mixture was stirred at rt for 2 h then the pH was adjusted to 2 by the addition of 2M aq. HCl. The mixture was diluted with water then it was extracted with DCM. The phases were separated, and the organic phase was washed with sat. aq. NaCl solution, dried over MgSO₄ and concentrated in vacuo. The crude material was purified by automated flash chromatography using DCM and EtOAc as eluents to afford the title product (3.14 g, 5.59 mmol, 64%).

¹H NMR (500 MHz, DMSO-d6) δ ppm 11.90-11.35 (br, 1H), 7.38-6.81 (m, 11H), 6.40-6.05 (m, 1H), 5.36-3.55 (m, 7H), 3.52-1.88 (m. 12H), 1.06-0.49 (m, 3H); ¹³C NMR (125 MHz, DMSO-d6) δ ppm 169.8, 168.4, 166.5/166.2, 131.0-124.8. 111.0/110.6, 48.2/42.9, 47.1, 44.0/43.3, 40.3/39.6, 34.3, 32.6, 32.1/31.8, 29.1/28.2, 18.6-16.4, 11.7/11.2. HRMS-ESI (m/z) [M+H]⁺ calcd for C₃₅H₃₆N₃O₄: 562.2700, found 562.2705

### Preparation IX: 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-2-[2-[2-fluoro-4-(2-morpholinoethoxy)phenyl]acetyl]-3,4-dihydro-1H-isoquinoline-7-carboxylic acid

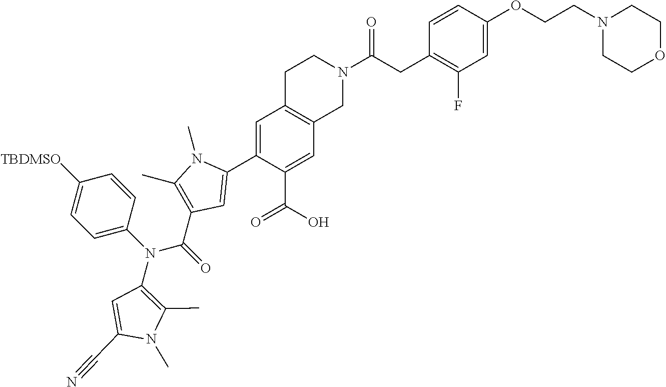 [SMILES: Cc1c(N(C(=O)c2cc(-c3cc4c(cc3C(=O)O)CN(C(=O)Cc3ccc(OCCN5CCOCC5)cc3F)CC4)n(C)c2C)c2ccc(O[Si](C)(C)C(C)(C)C)cc2)cc(C#N)n1C]

### Step A: 2-benzyloxycarbonyl-6-(4-carboxy-1,5-dimethyl-pyrrol-2-yl)-3,4-dihydro-1H-isoquinoline-7-carboxylic acid

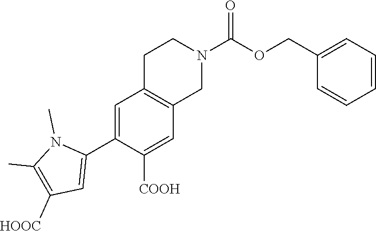 [SMILES: Cc1c(C(=O)O)cc(-c2cc3c(cc2C(=O)O)CN(C(=O)OCc2ccccc2)CC3)n1C]

To a suspension of Preparation I (20.01 g, 41.99 mmol, 1 eq.) in methanol (100 mL) and water (20 mL), NaOH (14.07 g, 352 mmol, 8.4 eq.) was added and mixture was stirred at reflux temperature for 2 days. The pH of the cooled mixture was adjusted to 6 by the addition of cc. aq. HCl then methanol was evaporated at reduced pressure. The resulting slurry was diluted with 2-propanol and the crude product was filtered out. This crude material was dissolved again in the mixture of ice-water (400 mL) and 2M aq. NaOH solution (41 mL, 82 mmol) then a solution of benzyl carbonochloridate (7.1 mL, 50.7 mmol, 1.19 eq.) in diethyl ether (100 mL) was added dropwise. The mixture was stirred for 24 h then the pH of the aqueous phase was adjusted to 2 by the addition of 2M aq. HCl and the resulting white precipitate was filtered off, washed with water and dried. The crude material was purified by automated flash chromatography using DCM and MeOH (1% NH₃) as eluents to afford the title product as a white solid (15.09 g, 32.2 mmol, 77%).

¹H NMR (500 MHz, DMSO-d6) δ ppm 7.71 (s, 1H), 7.44-7.11 (m, 5H), 7.16 (s, 1H), 6.19 (s, 1H), 5.14 (s, 2H), 4.71/4.65 (br/br., 2H), 3.67 (br., 2H), 3.21 (s, 3H), 2.86 (t, 2H), 2.48 (s, 3H); ¹³C NMR (125 MHz, DMSO-d6) δ ppm 168.5, 166.7, 155.1, 133.1, 128.2, 109.2, 66.8, 45.5, 41.4/41.1, 31.7, 28.6/28.4, 11.7.

HRMS-ESI (m/z) [M+H]⁺ calcd for C₂₅H₂₅N₂O₆: 449.1707, found 449.1708

### Step B: 5-(2-benzyloxycarbonyl-7-tert-butoxycarbonyl-3,4-dihydro-1H-isoquinolin-6-yl)-1,2-dimethyl-pyrrole-3-carboxylic acid

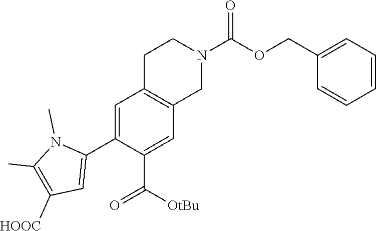 [SMILES: Cc1c(C(=O)O)cc(-c2cc3c(cc2C(=O)OC(C)(C)C)CN(C(=O)OCc2ccccc2)CC3)n1C]

A solution of the product from Step A (2.50 g, 5.57 mmol, 1 eq.) and 1,1-di-tert-butoxy-N,N-dimethyl-methanamine (13 mL, 55.7 mmol, 10 eq.) in THE (50 mL) was stirred at rt for 18 h. The reaction mixture was concentrated in vacuo and the crude material was purified by automated flash chromatography using heptane and EtOAc as eluents. The title product was isolated as a white solid (1.62 g, 3.21 mmol, 58%).

¹H NMR (500 MHz, DMSO-d6) δ ppm 11.58 (s, 1H), 7.62 (s, 3H), 7.45-7.29 (m, 5H), 7.16 (s, 1H), 6.16 (s, 1H), 5.14 (s, 2H), 4.71/4.64 (brs, 2H), 3.67 (brm, 2H), 3.2 (s, 3H), 2.86 (t, 2H), 2.49 (s, 3H), 1.25 (s, 9H).

HRMS-ESI (m/z) [M+H]⁺ calcd for C₂₉H₃₃N₂O₆: 505.2333, found 505.2335

### Step C: 02-benzyl 07-tert-butyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-3,4-dihydro-1H-isoquinoline-2,7-dicarboxylate

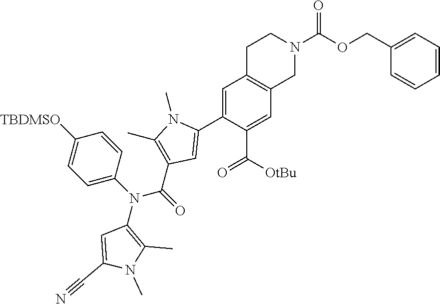 [SMILES: Cc1c(N(C(=O)c2cc(-c3cc4c(cc3C(=O)OC(C)(C)C)CN(C(=O)OCc3ccccc3)CC4)n(C)c2C)c2ccc(O[Si](C)(C)C(C)(C)C)cc2)cc(C#N)n1C]

Using General procedure 2a and the product from Step B (12.70 g, 25.2 mmol, 1 eq.) as the appropriate carboxylic acid and the product from Step B of Preparation IVa (10.30 g, 30.2 mmol, 1.2 eq.) as the appropriate aniline afforded the title product (14.2 g, 17.1 mmol, 68%).

¹H NMR (400 MHz, DMSO-d6) δ ppm 7.58 (s, 1H), 7.45-7.28 (m, 5H), 7.01 (dm, 2H), 6.9 (s, 1H), 6.76 (s, 1H), 6.73 (dm, 2H), 5.12 (s, 2H), 5.09 (s, 1H), 4.67/4.6 (br/br., 2H), 3.63 (br., 2H), 3.59 (s, 3H), 3.14 (s, 3H), 2.8 (t, 2H), 2.41 (s, 3H), 2.05 (s, 3H), 1.27 (s, 9H), 0.84 (s, 9H), 0.08 (s, 6H); ¹³C NMR (100 MHz, DMSO-d6) δ ppm 166.4, 155.1, 135.6, 133, 132.7, 130.8, 128.5, 128, 126.6, 120.5, 117.6, 114.4, 114, 108.6, 100.7, 66.8, 45.5, 41.4/41.1, 33.2, 31.4, 28.6/28.4, 27.8, 26, 12.1, 10.1, −4.2.

HRMS-ESI (m/z) [M+H]⁺ calcd for C₄₈H₅₈N₅O₆Si: 828.4151, found 828.4163

### Step D: tert-butyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-1,2,3,4-tetrahydroisoquinoline-7-carboxylate

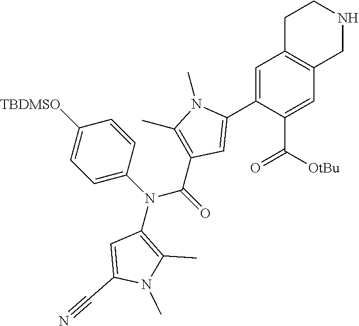 [SMILES: Cc1c(N(C(=O)c2cc(-c3cc4c(cc3C(=O)OC(C)(C)C)CNCC4)n(C)c2C)c2ccc(O[Si](C)(C)C(C)(C)C)cc2)cc(C#N)n1C]

According to General procedure 6b the product from Step C (1.00 g, 1.21 mmol, 1 eq.) in THE (13 mL) was hydrogenated using 26 mg Pd catalyst (10% on charcoal) affording the title product (0.836 g, 1.205 mmol, quant.).

¹H NMR (500 MHz, DMSO-d6) δ ppm 7.46/7.44 (s/s, 1H), 7.01 (d, 2H), 6.81 (s, 1H), 6.77 (s, 1H), 6.74/6.73 (d/d, 2H), 5.1 (s, 1H), 3.97 (s, 2H), 3.59 (s, 3H), 3.13/3.12 (s/s, 3H), 3.03 (t, 2H), 2.74 (t, 2H), 2.42/2.41 (s/s, 3H), 2.05/2.04 (s/s, 3H), 1.26 (s, 9H), 0.87 (s, 9H), 0.1 (s, 6H); ¹³C NMR (125 MHz, DMSO-d6) δ ppm 133.1, 128.4, 128, 120.5, 117.6, 108.3, 46.7, 42.6, 33.2, 31.4, 28, 27.8, 26, 12.1, 10.1, −4.2.

HRMS-ESI (m/z) [M+H]⁺ calcd for C₄₀H₅₂N₅O₄Si: 694.3783, found 694.3780

### Step E: tert-butyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-2-[2-[2-fluoro-4-(2-morpholinoethoxy)phenyl]acetyl]-3,4-dihydro-1H-isoquinoline-7-carboxylate

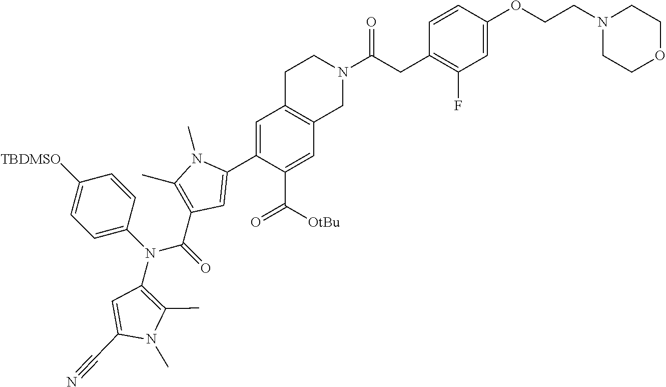 [SMILES: Cc1c(N(C(=O)c2cc(-c3cc4c(cc3C(=O)OC(C)(C)C)CN(C(=O)Cc3ccc(OCCN5CCOCC5)cc3F)CC4)n(C)c2C)c2ccc(O[Si](C)(C)C(C)(C)C)cc2)cc(C#N)n1C]

According to the amide coupling step of General Procedure 4c the product from Step C of Preparation Vb (0.511 g, 1.805 mmol, 1.5 eq.) in DMF (12 mL), TBTU (0.773 g, 2.406 mmol, 2 eq.), DIPEA (0.838 mL, 4.813 mmol, 4 eq.) and the product from Step D (0.835 g, 1.203 mmol, 1 eq.) was stirred at rt for 30 min. The reaction mixture was poured into water (120 mL) and the precipitates were filtered out, washed with water finally dried at 40° C. in vacuo to afford the title product as a yellow solid (1.145 g, 1.194 mmol, 99%).

¹H NMR (500 MHz, DMSO-d6) δ ppm 7.62-6.68 (m, 9H), 6.77 (s, 1H), 5.11/5.1 (s, 1H), 4.81/4.66 (s/s, 2H), 4.07 (t, 2H), 3.76/3.68 (t/t, 2H), 3.74 (s, 2H), 3.6/3.15/3.14 (s, 6H), 3.57 (br., 4H), 2.83/2.77 (t/t, 2H), 2.67 (t, 2H), 2.45 (br., 4H), 2.42/2.05 (s, 6H), 1.29/1.28 (s, 9H), 0.86/0.85 (s, 9H), 0.09 (s, 6H); ¹³C NMR (125 MHz, DMSO-d6) δ ppm 166.4, 117.6, 108.5, 66.6, 66.2, 57.4, 54.1, 46.7/43.9, 42.9/39.4, 33.4/33, 33.2/31.4, 29.1/28.3, 27.8, 26, 12.1/10.1, −4.2.

HRMS-ESI (m/z) [M+H]⁺ calcd for C₅₄H₆₈FN₆O₇Si: 959.4897, found 959.4897

### Step F. 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-2-[2-[2-fluoro-4-(2-morpholinoethoxy)phenyl]acetyl]-3,4-dihydro-1H-isoquinoline-7-carboxylic acid

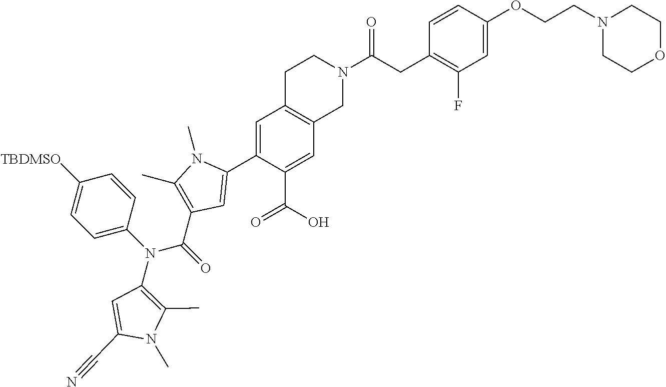 [SMILES: Cc1c(N(C(=O)c2cc(-c3cc4c(cc3C(=O)O)CN(C(=O)Cc3ccc(OCCN5CCOCC5)cc3F)CC4)n(C)c2C)c2ccc(O[Si](C)(C)C(C)(C)C)cc2)cc(C#N)n1C]

According to General Procedure 3a the product from Step E (1.145 g, 1.11 mmol, 1 eq.) in 1,4-dioxane (20 mL), trimethylsilyl trifluoromethanesulfonate (1.00 mL, 5.55 mmol, 5 eq.) and TEA (0.773 mL, 5.55 mmol, 5 eq) in 1,4-dioxane (8 mL) were reacted for 5 h to afford the title product as an orange solid (0.770 g, 0.724 mmol, 65%).

¹H NMR (400 MHz, DMSO-d6) δ ppm 7.71-6.61 (m, 9H), 6.77 (s, 1H), 5.11/5.1 (s, 1H), 4.81/4.66 (s/s, 2H), 4.07 (t, 2H), 3.76/3.68 (t/t, 2H), 3.74 (s, 2H), 3.57 (br., 4H), 3.56/3.15/3.14 (s, 6H), 2.83/2.77 (t/t, 2H), 2.67 (t, 2H), 2.45 (br., 4H), 2.38/1.99 (s, 6H), 0.86/0.85 (s, 9H), 0.09 (s, 6H); ¹³C NMR (100 MHz, DMSO-d6) δ ppm 168.3, 117.6, 108.5, 66.6, 66.2, 57.4, 54.1, 46.7/43.9, 42.9/39.4, 33.4/33, 33.2/31.4, 29.1/28.3, 26, 11.9/10.1, −4.2.

HRMS-ESI (m/z) [M+H]⁺ calcd for C₅₀H₆₀FN₆O₇Si: 903.4271, found 903.4269

### Preparation X: methyl 2-[4-(2-bromoethoxy)-2-fluoro-phenyl]acetate

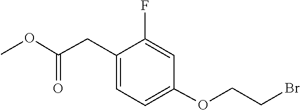 [SMILES: COC(=O)Cc1ccc(OCCBr)cc1F]

According to General Procedure 7b methyl 2-(2-fluoro-4-hydroxy-phenyl)acetate (2.000 g, 10.86 mmol, 1 eq.), 2-bromoethanol (4.343 g, 34.75 mmol, 3.2 eq.) were reacted to obtain the desired product (3.15 g, 10.8 mmol, quant.). ¹H NMR (500 MHz, DMSO-d6) δ ppm 7.25 (t, 1H), 6.86 (dd, 1H), 6.77 (dd, 1H), 4.33 (t, 2H), 3.8 (t, 2H), 3.65 (s, 2H), 3.61 (s, 3H); ¹³C NMR (125 MHz, DMSO-d6) δ ppm 171.4, 161.5, 158.8, 132.8, 114.3, 111.2, 102.6, 68.6, 52.3, 33.6, 31.7.

### EXAMPLES

### Example 1: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-[2-(3-methylphenyl)acetyl]-1,2,3,4-tetrahydroisoquinolin-6-yl}-1H-pyrrole-3-carboxamide

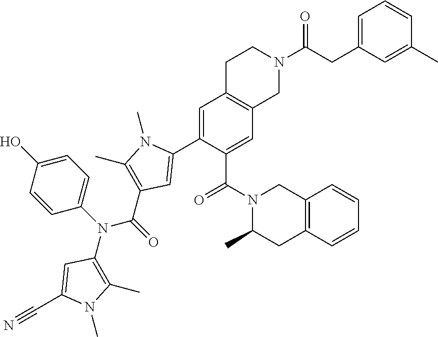 [SMILES: Cc1cccc(CC(=O)N2CCc3cc(-c4cc(C(=O)N(c5ccc(O)cc5)c5cc(C#N)n(C)c5C)c(C)n4C)c(C(=O)N4Cc5ccccc5C[C@H]4C)cc3C2)c1]

Using General procedure 4c, starting from 2-(m-tolyl)acetic acid (34.5 mg, 1.5 eq.) as the appropriate carboxylic acid and Preparation VIIb (117 mg, 0.15 mmol) as the appropriate amine, afforded the title compound (51.8 mg, 43%).

HRMS-ESI (m/z) [M+H]⁺ calcd for C₄₉H₄₈N₆O₄: 785.3737, found 785.3825.

### Example 2: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-phenoxyacetyl)-1,2,3,4-tetrahydroisoquinolin-6-yl}-1H-pyrrole-3-carboxamide

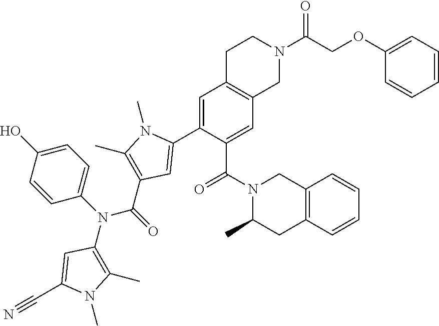 [SMILES: Cc1c(N(C(=O)c2cc(-c3cc4c(cc3C(=O)N3Cc5ccccc5C[C@H]3C)CN(C(=O)COc3ccccc3)CC4)n(C)c2C)c2ccc(O)cc2)cc(C#N)n1C]

Using General procedure 4d, starting from 2-phenoxyacetyl chloride (37 mg, 1.2 eq.) as the appropriate carboxylic acid chloride and Preparation VIIb (140 mg, 0.18 mmol) as the appropriate amine, afforded the title compound (67 mg, 73%).

HRMS-ESI (m/z) [M+H]⁺ calcd for C₄₈H₄₇N₆O₅: 787.3608, found 787.3604.

### Example 3: N-(5-cyano-1,2-dimethylpyrrol-3-yl)-1,2-dimethyl-5-(7-{[(3R)-3-methyl-3,4-dihydro-1H-isoquinolin-2-yl]carbonyl}-2-{2-[3-(4-methylpiperazin-1-yl)phenyl]acetyl}-3,4-dihydro-1H-isoquinolin-6-yl)-N-phenylpyrrole-3-carboxamide

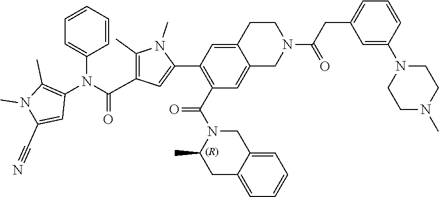

### Step A: methyl 2-[3-(4-methylpiperazin-1-yl)phenyl]acetate

A mixture of methyl 3-bromophenylacetate (536 μL, 3.27 mmol, 1 eq.), 1-methylpiperazine, (545 μL, 4.91 mmol, 1.5 eq.) and Cs₂CO₃ (2.13 g, 6.55 mmol, 2 eq.) in toluene (20 mL) was sparged with N₂. DavePhos (129 mg, 327 μmol, 0.1 eq.) and Pd₂(dba)₃ (150 mg, 163.7 μmol, 0.05 eq.) were added, the mixture was sparged again and then heated at 120° C. under N₂ for 20 h. The mixture was allowed to cool to rt and filtered through a celite cartridge, washing with EtOAc. The filtrate was washed with sat. aq. NaHCO₃ solution and brine, dried over MgSO₄ and concentrated in vacuo. Purification by automated flash chromatography eluting with a gradient of 0-7% MeOH in DCM afforded the title product (203 mg, 0.82 mmol, 25%).

¹H NMR (400 MHz, DMSO-d6) δ ppm: 7.17-7.10 (m, 1H), 6.84-6.78 (m, 2H), 6.68-6.63 (m, 1H), 3.60 (s, 3H), 3.59 (s, 2H), 3.14-3.07 (m, 4H), 2.47-2.40 (m, 4H), 2.22 (s, 3H).

### Step B: 2-[3-(4-methylpiperazin-1-yl)phenyl]acetic acid

Using General procedure 8 and the product from Step A (400 mg, 1.61 mmol, 1 eq.) as the appropriate ester afforded the title product (310 mg, 1.32 mmol, 82%).

LRMS calcd for C₁₃H₁₈N₂O₂: 234.1, found 235.0 [M+H]⁺

¹H NMR (400 MHz, DMSO-d6) δ ppm: 7.14-7.07 (m, 1H), 6.83-6.79 (m, 1H), 6.79-6.74 (m, 1H), 6.68-6.63 (m, 1H), 3.41 (s, 2H), 3.13-3.05 (m, 4H), 2.47-2.39 (m, 4H), 2.21 (s, 3H).

### Step C: tert-butyl 6-{4-[(5-cyano-1,2-dimethylpyrrol-3-yl)(phenyl)carbamoyl]-1,5-dimethylpyrrol-2-yl}-7-{[(3R)-3-methyl-3,4-dihydro-1H-isoquinolin-2-yl]carbonyl}-3,4-dihydro-1H-isoquinoline-2-carboxylate

Using General procedure 2a and Preparation Ha (1.2 g, 2.21 mmol, 1 eq.) as the appropriate acid and Example 65, Step A (933 mg, 4.41 mmol, 2 eq.) as the appropriate aniline afforded the title product (1.27 g, 1.72 mmol, 78%).

LRMS calcd for C₄₅H₄₈N₆O₄: 736.4, found 737.4 [M+H]⁺

### Step D: N-(5-cyano-1,2-dimethylpyrrol-3-yl)-1,2-dimethyl-5-(7-{[(3R)-3-methyl-3,4-dihydro-1H-isoquinolin-2-yl]carbonyl}-1,2,3,4-tetrahydroisoquinolin-6-yl)-N-phenylpyrrole-3-carboxamide hydrochloride

A solution of 3 M HCl in MeOH (15 mL) was added to the product from Step C (1.28 g, 1.74 mmol, 1 eq.) and the mixture stirred at rt for 5 h. The solvent was removed in vacuo and the residue dried to afford the title product, which was used directly in the subsequent step without further purification (1.17 g, 1.74 mmol, quant.).

LRMS calcd for C₄₀H₄₀N₆₂: 636.3, found 637.3 [M+H]⁺

### Step E: N-(5-cyano-1,2-dimethylpyrrol-3-yl)-1,2-dimethyl-5-(7-{[(3R)-3-methyl-3,4-dihydro-1H-isoquinolin-2-yl]carbonyl}-2-{2-[3-(4-methylpiperazin-1-yl)phenyl]acetyl}-3,4-dihydro-1H-isoquinolin-6-yl)-N-phenylpyrrole-3-carboxamide

Using General procedure 4b and the product from Step D (120 mg, 0.18 mmol, 1 eq.) as the appropriate amine and the product from Step B (50 mg, 0.21 mmol, 1.2 eq) as the appropriate carboxylic acid afforded the title product (46.2 mg, 0.05 mmol, 30%).

HRMS calcd for C₅₃H₅₆N₈O₃: 852.4475, found 853.4550 [M+H]⁺

### Example 4: N-[5-cyano-1-(2-hydroxyethyl)-2-methyl-pyrrol-3-yl]-1,2-dimethyl-5-[7-[(3R)-3-methyl-3,4-dihydro-1H-isoquinoline-2-carbonyl]-2-(2-phenylacetyl)-3,4-dihydro-1H-isoquinolin-6-yl]-N-phenyl-pyrrole-3-carboxamide

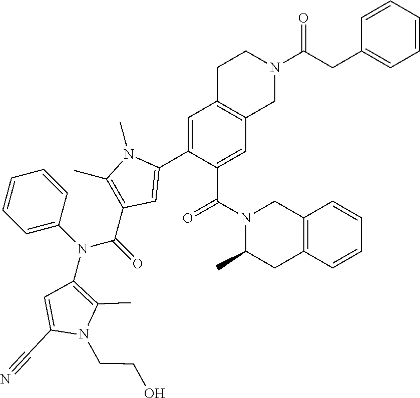 [SMILES: Cc1c(C(=O)N(c2ccccc2)c2cc(C#N)n(CCO)c2C)cc(-c2cc3c(cc2C(=O)N2Cc4ccccc4C[C@H]2C)CN(C(=O)Cc2ccccc2)CC3)n1C]

### Step A: 4-bromo-1-[2-[tert-butyl(dimethyl)silyl]oxyethyl]-5-methyl-pyrrole-2-carbonitrile

Using the General procedure 7c, starting from the 2-[tert-butyl(dimethyl)silyl]oxyethanol (705 mg, 2.0 eq.) as the appropriate alcohol, afforded the title product (625 mg, 91%).

¹H NMR (500 MHz, DMSO-d6) δ ppm 7.07 (s, 1H), 4.15 (t, 2H), 3.81 (t, 2H), 2.24 (s, 3H), 0.78 (s, 9H), −0.11 (s, 6H); ¹³C NMR (125 MHz, DMSO-d6) δ ppm 135.1, 120.6, 113.9, 103.5, 95.7, 62.1, 49.5, 26.1, 18.4, 11.3, −5.4.

HRMS-ESI (m/z) [M+H]⁺ calcd for C₁₄H₂₄BrN₂OSi: 343.0836, found 343.0843.

### Step B: 4-anilino-1-[2-[tert-butyl(dimethyl)silyl]oxyethyl]-5-methyl-pyrrole-2-carbonitrile

Using General procedure 1b, starting from the product of Step A (500 mg, 1.46 mmol) as the appropriate aryl bromide and aniline (0.199 mL, 1.5 eq.), afforded the title product (405 mg, 78%).

¹H NMR (500 MHz, DMSO-d6) δ ppm 7.17 (s, 1H), 7.05 (t, 2H), 6.78 (s, 1H), 6.57 (m, 2H), 6.57 (m, 1H), 4.12 (t, 2H), 3.85 (t, 2H), 2.10 (s, 3H), 0.82 (s, 9H), −0.05 (s, 6H); ¹³C NMR (125 MHz, DMSO-d6) δ ppm 148.3, 131.5, 129.3, 123.6, 117.1, 116.5, 114.9, 113.1, 100.6, 62.4, 48.7, 26.2, 18.4, 10.0, −5.3.

HRMS-ESI (m/z) [M+H]⁺ calcd for C₂₀H₃₀N₃OSi: 356.2153, found 356.2142.

### Step C. N-[5-cyano-1-(2-hydroxyethyl)-2-methyl-pyrrol-3-yl]-1,2-dimethyl-5-[7-[(3R)-3-methyl-3,4-dihydro-1H-isoquinoline-2-carbonyl]-2-(2-phenylacetyl)-3,4-dihydro-1H-isoquinolin-6-yl]-N-phenyl-pyrrole-3-carboxamide

Using General procedure 2b, starting from the Preparation VIII as the appropriate acid (200 mg, 0.36 mmol) and the product of Step B (152 mg, 1.2 eq.) as the appropriate aniline followed by General procedure 3c for silyl deprotection the title compound was obtained (25 mg, 9%).

HRMS-ESI (m/z) [M+H]⁺ calcd for C₄₉H₄₉N₆O₄: 785.3810, found 785.3814.

### Example 5: N-(5-cyano-1,2-dimethyl-1H-pyrrol-3-yl)-5-{2-[2-(4-fluorophenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

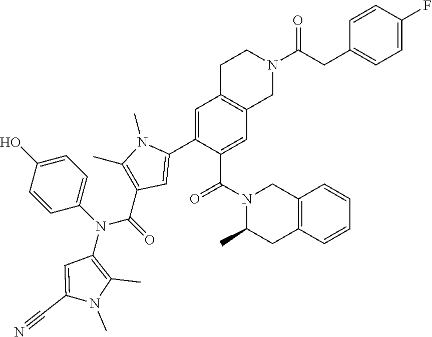 [SMILES: Cc1c(N(C(=O)c2cc(-c3cc4c(cc3C(=O)N3Cc5ccccc5C[C@H]3C)CN(C(=O)Cc3ccc(F)cc3)CC4)n(C)c2C)c2ccc(O)cc2)cc(C#N)n1C]

Using General procedure 4d, starting from 2-(4-fluorophenyl)acetyl chloride (36 mg, 1.1 eq.) as the appropriate carboxylic acid chloride and Preparation VIIb (145 mg, 0.19 mmol) as the appropriate amine, afforded the title compound (41.6 mg, 28%).

HRMS-ESI (m/z) [M+H]⁺ calcd for C₄₈H₄₆FN₆O₄: 789.3564, found 789.3560.

### Example 6: N-(5-cyano-1,2-dimethylpyrrol-3-yl)-N-(4-hydroxyphenyl)-1,2-dimethyl-5-(7-{[(3R)-3-methyl-3,4-dihydro-1H-isoquinolin-2-yl]carbonyl}-2-{2-[3-(morpholin-4-yl)phenyl]acetyl}-3,4-dihydro-1H-isoquinolin-6-yl)pyrrole-3-carboxamide

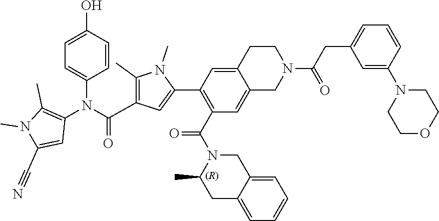

### Step A: methyl 2-[3-(morpholin-4-yl)phenyl]acetate

A mixture of methyl 3-bromophenylacetate (0.36 mL, 2.18 mmol, 1 eq.), morpholine (0.29 mL, 3.27 mmol, 1.5 eq.) and Cs₂CO₃ (1.42 g, 4.37 mmol, 2 eq.) in toluene (10 mL) was sparged with N₂. DavePhos (86 mg, 0.22 mmol, 0.1 eq.) and Pd₂(dba)₃ (100 mg, 0.11 mmol, 0.05 eq.) were added, the mixture was sparged again and then heated at 120° C. under N₂ for 20 h. The mixture was allowed to cool to rt and filtered through a celite cartridge, washing with EtOAc. The filtrate was washed with sat. aq. NaHCO₃ solution and brine, dried over MgSO₄ and concentrated in vacuo. Purification by automated flash chromatography eluting with a gradient of 0-50% EtOAc in heptane afforded the title product (359 mg, 1.53 mmol, 70%).

LRMS calcd for C₁₃H₁₇NO₃: 235.1, found 236.0 [M+H]⁺

¹H NMR (400 MHz, DMSO-d6) δ ppm: 7.19-7.13 (m, 1H), 6.85-6.80 (m, 2H), 6.71-6.67 (m, 1H), 3.75-3.68 (m, 4H), 3.60 (s, 3H), 3.60 (s, 2H), 3.11-3.05 (m, 4H).

### Step B: 2-[3-(morpholin-4-yl)phenyl]acetic acid

Using General procedure 8 and the product from Step A (359 mg, 1.53 mmol, 1 eq.) as the appropriate ester afforded the title product (174 mg, 0.79 mmol, 52%).

LRMS calcd for C₁₂H₁₅NO₃: 221.1, found 222.0 [M+H]⁺

¹H NMR (400 MHz, DMSO-d6) δ ppm: 7.30-7.20 (m, 1H), 7.13-6.96 (m, 2H), 6.94-6.80 (m, 1H), 3.85-3.77 (m, 4H), 3.53 (s, 2H), 3.25-3.15 (m, 4H).

### Step C: N-(5-cyano-1,2-dimethylpyrrol-3-yl)-N-(4-hydroxyphenyl)-1,2-dimethyl-5-(7-{[(3R)-3-methyl-3,4-dihydro-1H-isoquinolin-2-yl]carbonyl}-2-{2-[3-(morpholin-4-yl)phenyl]acetyl}-3,4-dihydro-1H-isoquinolin-6-yl)pyrrole-3-carboxamide

Using General procedure 4b and Preparation IVa (120 mg, 0.17 mmol, 1 eq.) as the appropriate amine and the product from Step B (46 mg, 0.21 mmol, 1.2 eq.) as the appropriate carboxylic acid afforded the title product (61.3 mg, 0.07 mmol, 41%).

HRMS calcd for C₅₂H₅₃N₇O₅: 855.4108, found 856.4181 [M+H]⁺

### Example 7: N-(5-cyano-1,2-dimethyl-1H-pyrrol-3-yl)-N-(4-hydroxyphenyl)-5-{2-[2-(3-methoxyphenyl)acetyl]-7-[(3R)-3-methyl-1,2,3,4-tetrahydroisoquinoline-2-carbonyl]-1,2,3,4-tetrahydroisoquinolin-6-yl}-1,2-dimethyl-1H-pyrrole-3-carboxamide

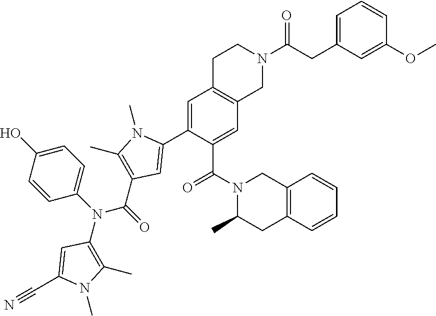 [SMILES: COc1cccc(CC(=O)N2CCc3cc(-c4cc(C(=O)N(c5ccc(O)cc5)c5cc(C#N)n(C)c5C)c(C)n4C)c(C(=O)N4Cc5ccccc5C[C@H]4C)cc3C2)c1]

Using General procedure 4d, starting from 2-(3-methoxyphenyl)acetyl chloride (45 mg, 1.2 eq.) as the appropriate carboxylic acid chloride and Preparation VIIb (155 mg, 0.20 mmol) as the appropriate amine, afforded the title compound (30 mg, 19%).

HRMS-ESI (m/z) [M+H]⁺ calcd for C₄₉H₄₉N₆O₅: 801.3764, found 801.3764.

### Example 8: 5-[2-(2-{3-[(9aS)-hexahydro-1H-piperazino[2,1-c]morpholin-8-yl]phenyl}acetyl)-7-{[(3R)-3-methyl-3,4-dihydro-1H-isoquinolin-2-yl]carbonyl}-3,4-dihydro-1H-isoquinolin-6-yl]-N-(5-cyano-1,2-dimethylpyrrol-3-yl)-1,2-dimethyl-N-phenylpyrrole-3-carboxamide

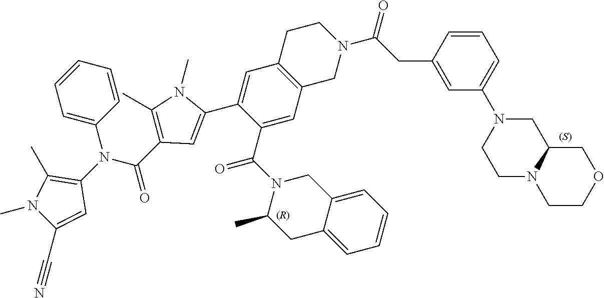

### Step A: methyl 2-{3-[(9aS)-hexahydro-1H-piperazino[2,1-c]morpholin-8-yl]phenyl}acetate

Methyl 3-bromophenylacetate (500 mg, 2.18 mmol, 1 eq.), (9aS)-octahydropiperazino[2,1-c]morpholine dihydrochloride (704 mg, 3.27 mmol, 1.5 eq.) and Cs₂CO₃ (2.84 g, 8.73 mmol, 4 eq.) were combined in toluene (10 mL) and the mixture sparged with N₂. DavePhos (86 mg, 0.22 mmol, 0.1 eq.) and Pd₂dba₃ (100 mg, 0.11 mmol, 0.05 eq.) were added and the mixture sparged with N₂ and then heated at 120° C. under microwave irradiation for 1 h. The mixture was allowed to cool to rt and filtered through a celite cartridge, washing with EtOAc. The filtrate was washed with sat. aq. NaHCO₃ solution and brine solution and then dried over MgSO₄ and concentrated in vacuo. Purification by automated flash chromatography eluting with a gradient of 0-100% EtOAc in heptane and then 0-32% MeOH in EtOAc afforded the title product (73 mg, 0.25 mmol, 12%).

LRMS calcd for C₁₆H₂₂N₂O₃: 290.2, found 291.0 [M+H]⁺

¹H NMR (400 MHz, MeOD) δ ppm: 7.21-7.14 (m, 1H), 6.88-6.82 (m, 2H), 6.77-6.73 (m, 1H), 3.87-3.81 (m, 1H), 3.80-3.74 (m, 1H), 3.71-3.63 (m, 4H), 3.63-3.55 (m, 3H), 3.47-3.41 (m, 1H), 3.32-3.24 (m, 1H), 2.90-2.81 (m, 2H), 2.77-2.69 (m, 1H), 2.49-2.32 (m, 4H).

### Step B: 2-{3-[(9aS)-hexahydro-1H-piperazino[2,1-c]morpholin-8-yl]phenyl}acetic acid

To a solution of the product from Step A (73 mg, 0.25 mmol, 1 eq.) in MeOH (15 mL) was added 2 M aq. NaOH solution (0.25 mL, 0.5 mmol, 2 eq.) and the mixture was heated at 80° C. under microwave irradiation for 0.5 h. The solvent was removed under reduced pressure and the residue was diluted with water, the pH adjusted to 2 using 2 M aq. HCl solution, and then freeze-dried to afford the title product, which was used directly in the subsequent step without further purification (69 mg, 0.25 mmol, 99%).

LRMS calcd for C₁₅H₂₀N₂O₃: 276.1, found 277.0 [M+H]⁺

¹H NMR (400 MHz, DMSO-d6) δ ppm: 12.40 (br s, 1H), 7.23-7.15 (m, 1H), 6.93-6.84 (m, 2H), 6.80-6.72 (m, 1H), 4.07-3.92 (m, 3H), 3.87-3.68 (m, 3H), 3.56-3.39 (m, 4H), 3.39-3.07 (m, 4H), 2.98-2.87 (m, 1H).

### Step C: 5-[2-(2-{3-[(9aS)-hexahydro-1H-piperazino[2,1-c]morpholin-8-yl]phenyl}acetyl)-7-{[(3R)-3-methyl-3,4-dihydro-1H-isoquinolin-2-yl]carbonyl}-3,4-dihydro-1H-isoquinolin-6-yl]-N-(5-cyano-1,2-dimethylpyrrol-3-yl)-1,2-dimethyl-N-phenylpyrrole-3-carboxamide

Using General procedure 4b and Example 3, Step D (100 mg, 0.15 mmol, 1 eq.) as the appropriate amine and the product from Step B (57 mg, 0.21 mmol, 1.4 eq.) as the appropriate carboxylic acid afforded the title product (22.5 mg, 0.03 mmol, 17%).

HRMS calcd for C₅₅H₅₈N₈O₄: 894.4581, found 895.4669 [M+H]⁺

### Example 9: N-(5-cyano-1,2-dimethyl-1H-pyrrol-3-yl)-N-(4-hydroxy-2-methylphenyl)-1,2-dimethyl-5-{7-[(3R)-3-methyl-1,2,3,4-tetrahydroisoquinoline-2-carbonyl]-2-(2-phenylacetyl)-1,2,3,4-tetrahydroisoquinolin-6-yl}-1H-pyrrole-3-carboxamide

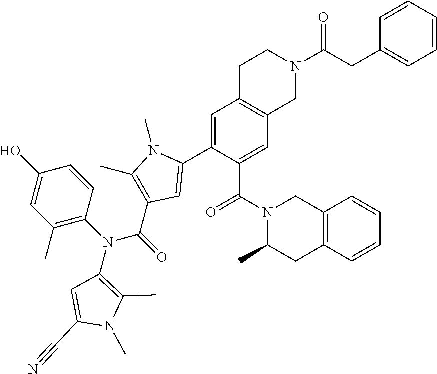 [SMILES: Cc1cc(O)ccc1N(C(=O)c1cc(-c2cc3c(cc2C(=O)N2Cc4ccccc4C[C@H]2C)CN(C(=O)Cc2ccccc2)CC3)n(C)c1C)c1cc(C#N)n(C)c1C]

### Step A: 4-[4-[tert-butyl(dimethyl)silyl]oxy-2-methyl-anilino]-1,5-dimethyl-pyrrole-2-carbonitrile

Using General procedure 1b, starting from 4-bromo-1,5-dimethyl-pyrrole-2-carbonitrile (430 mg, 2.16 mmol) as the appropriate aryl bromide and 4-[tert-butyl(dimethyl)silyl]oxy-2-methyl-aniline (615 mg, 1.2 eq.) as the appropriate aniline, afforded the title product (88 mg, 12%).

¹H NMR (500 MHz, DMSO-d6) δ ppm 6.67 (s, 1H), 6.55 (d, 1H), 6.42 (dd, 1H), 6.22 (d, 1H), 6.06 (s, 1H), 3.62 (s, 3H), 2.14 (s, 3H), 2.04 (s, 3H), 0.92 (s, 9H), 0.11 (s, 6H); ¹³C NMR (125 MHz, DMSO-d6) δ ppm 146.9, 140.6, 131.6, 125.1, 124.6, 122.1, 117.6, 116.3, 115.0, 113.2, 99.9, 33.2, 26.1, 18.3, 9.9, −4.0.

HRMS-ESI (m/z) [M+H]⁺ calcd for C₂₀H₃₀N₃OSi: 356.2153, found 356.2152.

### Step B: N-(5-cyano-1,2-dimethyl-1H-pyrrol-3-yl)-N-(4-hydroxy-2-methylphenyl)-1,2-dimethyl-5-{7-[(3R)-3-methyl-1,2,3,4-tetrahydroisoquinoline-2-carbonyl]-2-(2-phenylacetyl)-1,2,3,4-tetrahydroisoquinolin-6-yl}-1H-pyrrole-3-carboxamide

Using General procedure 2b, starting from Preparation VIII as the appropriate acid (120 mg, 0.214 mmol) and the product from Step A (84 mg, 1.1 eq.) as the appropriate amine, followed by General procedure 3c for silyl deprotection, the title compound was obtained (20 mg, 12%).

HRMS-ESI (m/z) [M+H]⁺ calcd for C₄₉H₄₉N₆O₄: 785.3810, found 785.3812.

### Example 10: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-[2-(4-phenoxyphenyl)acetyl]-1,2,3,4-tetrahydroisoquinolin-6-yl}-1H-pyrrole-3-carboxamide

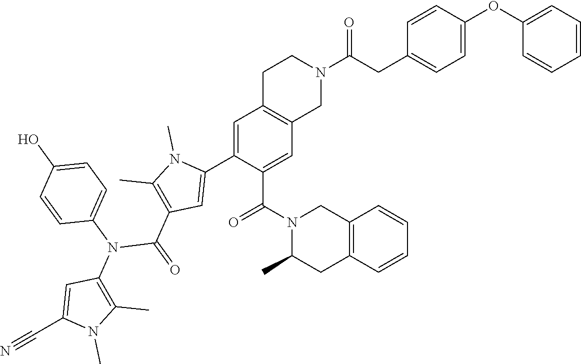 [SMILES: Cc1c(N(C(=O)c2cc(-c3cc4c(cc3C(=O)N3Cc5ccccc5C[C@H]3C)CN(C(=O)Cc3ccc(Oc5ccccc5)cc3)CC4)n(C)c2C)c2ccc(O)cc2)cc(C#N)n1C]

Using General procedure 4c, starting from 2-(4-phenoxyphenyl)acetic acid (70 mg, 2.4 eq.) as the appropriate carboxylic acid and Preparation VIIb (97 mg, 0.13 mmol) as the appropriate amine, afforded the title compound (35 mg, 32%).

HRMS-ESI (m/z) [M+H]⁺ calcd for C₅₄H₅₁N₆O₅: 863.3915, found 863.3921.

### Example 11: N-(5-cyano-1,2-dimethyl-1H-pyrrol-3-yl)-5-{2-[2-(2,4-difluorophenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

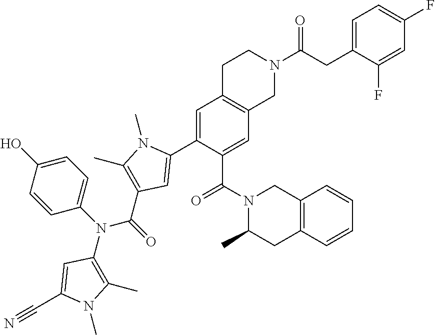 [SMILES: Cc1c(N(C(=O)c2cc(-c3cc4c(cc3C(=O)N3Cc5ccccc5C[C@H]3C)CN(C(=O)Cc3ccc(F)cc3F)CC4)n(C)c2C)c2ccc(O)cc2)cc(C#N)n1C]

Using General procedure 4c, starting from 2,4-difluorophenylacetic acid (33.7 mg, 1.5 eq.) as the appropriate carboxylic acid and Preparation VIIb (100 mg, 0.13 mmol) as the appropriate amine, afforded the title compound (60 mg, 57%).

HRMS-ESI (m/z) [M+H]⁺ calcd for C₄₈H₄₅F₂N₆O₄: 807.3465, found 807.3468.

### Example 12: N-(5-cyano-1,2-dimethyl-1H-pyrrol-3-yl)-5-{2-[2-(2-cyanophenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

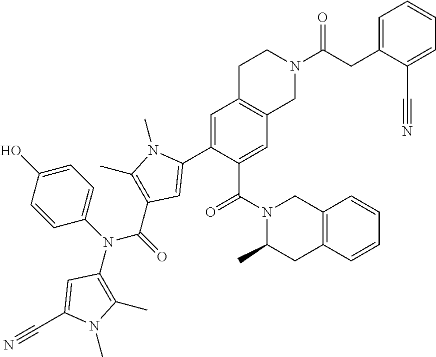 [SMILES: Cc1c(N(C(=O)c2cc(-c3cc4c(cc3C(=O)N3Cc5ccccc5C[C@H]3C)CN(C(=O)Cc3ccccc3C#N)CC4)n(C)c2C)c2ccc(O)cc2)cc(C#N)n1C]

Using General procedure 4c, starting from 2-(2-cyanophenyl)acetic acid (31.5 mg, 1.5 eq.) as the appropriate carboxylic acid and Preparation VIIb (100 mg, 0.13 mmol) as the appropriate amine, afforded the title compound (40 mg, 39%).

HRMS-ESI (m/z) [M+H]⁺ calcd for C₄₉H₄₆N₇O₄: 796.3606, found 796.3609.

### Example 13: N-(5-cyano-1,2-dimethyl-1H-pyrrol-3-yl)-N-(2-fluoro-4-hydroxyphenyl)-1,2-dimethyl-5-{7-[(3R)-3-methyl-1,2,3,4-tetrahydroisoquinoline-2-carbonyl]-2-(2-phenylacetyl)-1,2,3,4-tetrahydroisoquinolin-6-yl}-1H-pyrrole-3-carboxamide

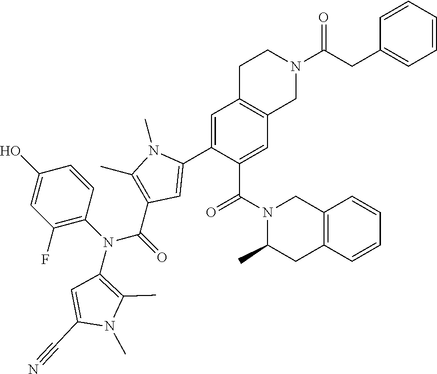 [SMILES: Cc1c(N(C(=O)c2cc(-c3cc4c(cc3C(=O)N3Cc5ccccc5C[C@H]3C)CN(C(=O)Cc3ccccc3)CC4)n(C)c2C)c2ccc(O)cc2F)cc(C#N)n1C]

### Step A: 4-[tert-butyl(dimethyl)silyl]oxy-2-fluoro-aniline

A solution of tert-butyl-chloro-dimethyl-silane (3.9 g, 1.1 eq.) in THF (7 mL) was added dropwise to the solution of 4-amino-3-fluoro-phenol (3.0 g, 23.6 mmol, 2.33 mL) and imidazole (2.41 g, 1.5 eq.) in THE (30 mL). After the conversion was complete, the volatiles of the reaction mixture were removed by evaporation. Water (20 mL) was added and the mixture was extracted with Et₂O (2×20 mL). The combined organic layers were washed with brine, dried over MgSO₄ then concentrated. The residue was purified by column chromatography (heptane:EtOAc=9:1) to give the title compound (3.2 g, 55.8%).

¹H NMR (500 MHz, DMSO-d6) δ ppm 6.64 (dd, 1H), 6.52 (dd, 1H), 6.42 (ddd, 1H), 4.65 (s, 2H), 0.92 (s, 9H), 0.12 (s, 6H); ¹³C NMR (125 MHz, DMSO-d6) δ ppm 150.8, 145.8, 130.9, 117, 116.1, 107.8, 26.1, −4.2.

HRMS-ESI (m/z) [M+H]⁺ calcd for C₁₂H₂₁FNOSi: 242.1371, found 242.1373.

### Step B: 4-[4-[tert-butyl(dimethyl)silyl]oxy-2-fluoro-anilino]-1,5-dimethyl-pyrrole-2-carbonitrile

Using General procedure 1b, starting from the product from Step A of Preparation IVa (430 mg, 2.16 mmol) as the appropriate aryl bromide and product from Step A (625.7 mg, 1.2 eq.) as the appropriate aniline, afforded the title product (95.7 mg, 12%).

¹H NMR (500 MHz, DMSO-d6) δ ppm 6.72 (s, 1H), 6.70 (br., 1H), 6.62 (dd, 1H), 6.44 (dd, 1H), 6.39 (t, 1H), 3.62 (s, 3H), 2.07 (s, 3H), 0.92 (s, 9H), 0.14 (s, 6H); ¹³C NMR (125 MHz, DMSO-d6) δ ppm 151.5, 146.6, 131.8, 130.6, 123.6, 116.2, 116, 114.8, 114.5, 108.0, 100.1, 33.2, 26, 9.9, −4.2.

HRMS-ESI (m/z) [M+H]⁺ calcd for C₁₉H₂₇FN₃OSi: 360.1902, found 360.1904.

### Step C: N-(5-cyano-1,2-dimethyl-1H-pyrrol-3-yl)-N-(2-fluoro-4-hydroxyphenyl)-1,2-dimethyl-5-{7-[(3R)-3-methyl-1,2,3,4-tetrahydroisoquinoline-2-carbonyl]-2-(2-phenylacetyl)-1,2,3,4-tetrahydroisoquinolin-6-yl}-1H-pyrrole-3-carboxamide

Using General procedure 2b, starting from Preparation VIII as the appropriate acid (120 mg, 0.214 mmol) and the product of Step B (84 mg, 1.1 eq.) as the appropriate amine, followed by General procedure 3c for silyl deprotection, the desired product was obtained (20 mg, 14%).

HRMS-ESI (m/z) [M+H]⁺ calcd for C₄₈H₄₆FN₆O₄: 789.3559, found 789.3562.

### Example 14: 5-[2-benzylsulfonyl-7-[(3R)-3-methyl-3,4-dihydro-1H-isoquinoline-2-carbonyl]-3,4-dihydro-1H-isoquinolin-6-yl]-N-(5-cyano-1,2-dimethyl-pyrrol-3-yl)-N-(4-hydroxyphenyl)-1,2-dimethyl-pyrrole-3-carboxamide

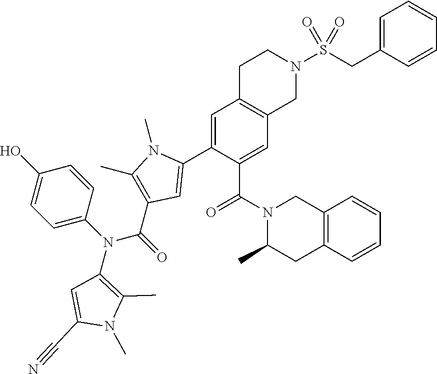 [SMILES: Cc1c(N(C(=O)c2cc(-c3cc4c(cc3C(=O)N3Cc5ccccc5C[C@H]3C)CN(S(=O)(=O)Cc3ccccc3)CC4)n(C)c2C)c2ccc(O)cc2)cc(C#N)n1C]

To a solution of Preparation VIIb (100 mg, 0.13 mmol) in DCE (2 mL) was added DIPEA (37 mg, 2.2 eq.) and phenylmethanesulfonyl chloride (40 mg, 1.6 eq.) and the mixture was stirred until complete conversion was observed. The TBDMS protecting group was removed using 2 M aq. NaOH (according to the protocol described in General procedure 4c) and the desired product was obtained (32 mg, 30%).

HRMS-ESI (m/z) [M+H]⁺ calcd for C₄₇H₄₆N₆O₅S: 807.3323, found 807.3325.

### Example 15: N-(5-cyano-1,2-dimethyl-1H-pyrrol-3-yl)-N-(4-hydroxyphenyl)-5-[2-(1H-indazole-3-carbonyl)-7-[(3R)-3-methyl-1,2,3,4-tetrahydroisoquinoline-2-carbonyl]-1,2,3,4-tetrahydroisoquinolin-6-yl]-1,2-dimethyl-1H-pyrrole-3-carboxamide

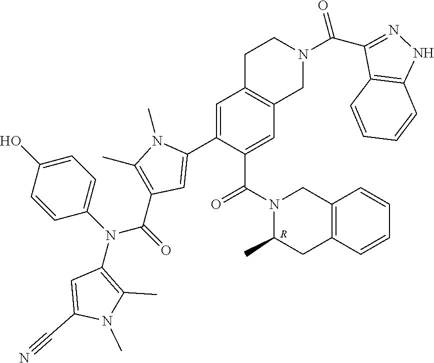

Using General procedure 4c, starting from 1H-indazole-3-carboxylic acid (48 mg, 1.5 eq.) as the appropriate carboxylic acid and Preparation VIIb (150 mg, 0.20 mmol) as the appropriate amine, afforded the title compound (50 mg, 32%).

HRMS-ESI (m/z) [M+H]⁺ calcd for C₄₈H₄₅N₈O₄: 797.3558, found 797.3560.

### Example 17: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-phenyl-1,3-thiazole-5-carbonyl)-1,2,3,4-tetrahydroisoquinolin-6-yl}-1H-pyrrole-3-carboxamide

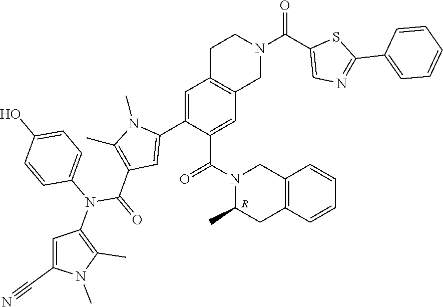

Using General procedure 4c, starting from 2-phenylthiazole-5-carboxylic acid (60 mg, 1.5 eq.) as the appropriate carboxylic acid and Preparation VIIb (150 mg, 0.20 mmol) as the appropriate amine, afforded the title compound as a white powder (39.7 mg, 24%).

HRMS-ESI (m/z) [M+H]⁺ calcd for C₅₀H₄₆N₇O₄S: 840.3326, found 840.3323.

### Example 18: N-(5-cyano-1,2-dimethylpyrrol-3-yl)-5-[2-(2,2-difluoro-2-phenylacetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

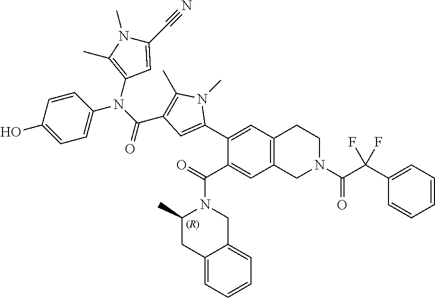

Using General procedure 4a and Preparation IVa (110 mg, 0.16 mmol, 1 eq.) as the appropriate amine and 2,2-difluoro-2-phenylacetic acid (27 mg, 0.16 mmol, 1 eq.) as the appropriate carboxylic acid afforded the title product (15.8 mg, 0.02 mmol, 12%).

HRMS calcd for C₄₈H₄₄F₂N₆O₄: 806.3392, found 807.3466 [M+H]⁺

### Example 19: 5-{2-[2-(4-chloro-2-fluorophenyl)acetyl]-7-[(3R)-3-methyl-1,2,3,4-tetrahydroisoquinoline-2-carbonyl]-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

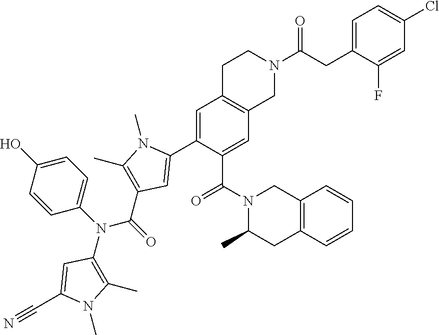 [SMILES: Cc1c(N(C(=O)c2cc(-c3cc4c(cc3C(=O)N3Cc5ccccc5C[C@H]3C)CN(C(=O)Cc3ccc(Cl)cc3F)CC4)n(C)c2C)c2ccc(O)cc2)cc(C#N)n1C]

Using General procedure 4c, starting from 2-(4-chloro-2-fluoro-phenyl)acetic acid (50 mg, 2.0 eq.) as the appropriate carboxylic acid and Preparation VIIb (100 mg, 0.13 mmol) as the appropriate amine, afforded the title compound (40 mg, 37%).

HRMS-ESI (m/z) [M+H]⁺ calcd for C₄₈H₄₅ClFN₆O: 823.3169, found 823.3176.

### Example 20: N-(5-cyano-1,2-dimethylpyrrol-3-yl)-5-{2-[(2,3-dihydro-1-benzofuran-2-yl)carbonyl]-7-{[(3R)-3-methyl-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

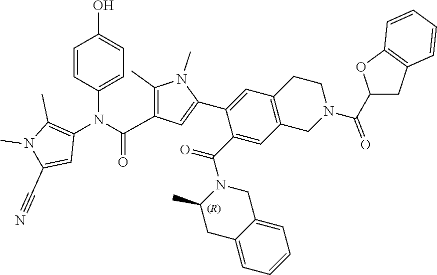

Using General procedure 4a and Preparation IVa (120 mg, 0.18 mmol, 1.2 eq.) as the appropriate amine and 2,3-dihydrobenzo[b]furan-2-carboxylic acid (25 mg, 0.15 mmol, 1 eq.) as the appropriate carboxylic acid afforded the title product (75 mg, 0.09 mmol, 63%).

HRMS calcd for C₄₉H₄₆N₆O₅: 798.3530, found 799.3610 [M+H]⁺

### Example 21: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(morpholin-4-yl)ethoxy]phenyl}acetyl)-1,2,3,4-tetrahydroisoquinolin-6-yl}-1H-pyrrole-3-carboxamide

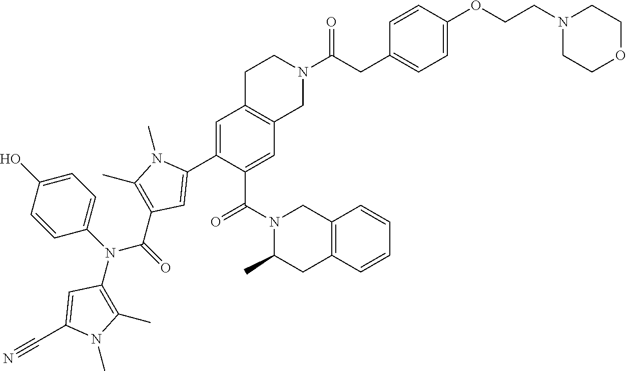 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(O)cc2)cc(C#N)n1C]

Using General procedure 4c, starting from the product of Preparation Va (80 mg, 2.5 eq.) as the appropriate carboxylic acid and Preparation VIIb (80 mg, 0.10 mmol) as the appropriate amine, afforded the title compound (59 mg, 63%).

HRMS-ESI (m/z) [M+H]⁺ calcd for C₅₄H₅₁N₇O₆: 900.4443, found 900.4446.

### Example 22: N-[5-cyano-1-[2-[cyclopropyl(methyl)amino]ethyl]-2-methyl-pyrrol-3-yl]-N-(4-hydroxyphenyl)-1,2-dimethyl-5-[7-[(3R)-3-methyl-3,4-dihydro-1H-isoquinoline-2-carbonyl]-2-(2-phenylacetyl)-3,4-dihydro-1H-isoquinolin-6-yl]pyrrole-3-carboxamide

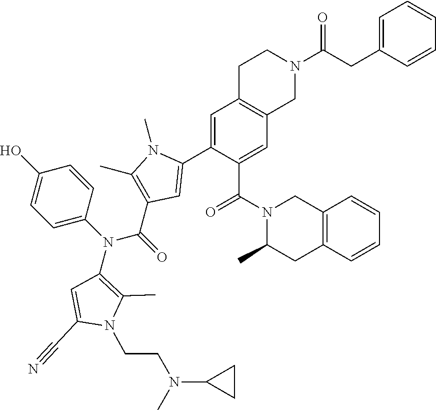 [SMILES: Cc1c(C(=O)N(c2ccc(O)cc2)c2cc(C#N)n(CCN(C)C3CC3)c2C)cc(-c2cc3c(cc2C(=O)N2Cc4ccccc4C[C@H]2C)CN(C(=O)Cc2ccccc2)CC3)n1C]

### Step A: 4-bromo-1-[2-[cyclopropyl(methyl)amino]ethyl]-5-methyl-pyrrole-2-carbonitrile

Using the General procedure 7c, starting from the 2-[cyclopropyl(methyl)amino]ethanol (1.2 g, 2.0 eq.) as the appropriate alcohol, afforded the title product (690 mg, 45%).

¹H NMR (500 MHz, DMSO-d6) δ ppm 7.05 (s, 1H), 4.06 (t, 2H), 2.74 (t, 2H), 2.31 (s, 3H), 2.23 (s, 3H), 1.78 (qn, 1H), 0.34 (m, 2 h), −0.02 (m, 2H).

LRMS (m/z) [M+H]⁺ calcd for C₁₂H₁₇BrN₃: 282.1, found 282.0.

### Step B: 4-[4-[tert-butyl(dimethyl)silyl]oxyanilino]-1-[2-[cyclopropyl(methyl)amino]ethyl]-5-methyl-pyrrole-2-carbonitrile

Using General procedure 1b, starting from the product of Step A (640 mg, 2.27 mmol) as the appropriate aryl bromide and Preparation IIIa (760 mg, 1.5 eq.) as the appropriate aniline, afforded the title compound (680 mg, 70%).

¹H NMR (500 MHz, DMSO-d6) δ ppm 6.87 (s, 1H), 6.72 (s, 1H), 6.59 (m, 2H), 6.46 (m, 2H), 4.02 (t, 2H), 2.77 (t, 2H), 2.33 (s, 3H), 2.07 (s, 3H), 1.79 (m, 1H), 0.92 (s, 9H), 0.37/0.06 (m+m, 4H), 0.12 (s, 6H); ¹³C NMR (125 MHz, DMSO-d6) δ ppm 130.7, 124.6, 120.6, 115.9, 114.9, 114.1, 99.9, 57.8, 44.8, 42.3, 39.0, 26.1, 9.9, 7.3, −4.1.

HRMS-ESI (m/z) [M+H]⁺ calcd for C₂₄H₃₇N₄OSi: 425.2731, found 425.2741.

### Step C. N-[5-cyano-1-[2-[cyclopropyl(methyl)amino]ethyl]-2-methyl-pyrrol-3-yl]-N-(4-hydroxyphenyl)-1,2-dimethyl-5-[7-[(3R)-3-methyl-3,4-dihydro-1H-isoquinoline-2-carbonyl]-2-(2-phenylacetyl)-3,4-dihydro-1H-isoquinolin-6-yl]pyrrole-3-carboxamide

Using General procedure 2b, starting from Preparation VIII (210 mg, 0.37 mmol) as the appropriate acid and the product of Step B (230 mg, 1.4 eq.) as the appropriate aniline, followed by General procedure 3c for silyl deprotection the title compound was obtained (75 mg, 23%).

HRMS-ESI (m/z) [M+H]⁺ calcd for C₅₃H₅₆N₇O₄: 854.4388, found 854.4385.

### Example 23: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[4-(2,2,2-trifluoroethyl)piperazin-1-yl]phenyl}acetyl)-1,2,3,4-tetrahydroisoquinolin-6-yl}-1H-pyrrole-3-carboxamide

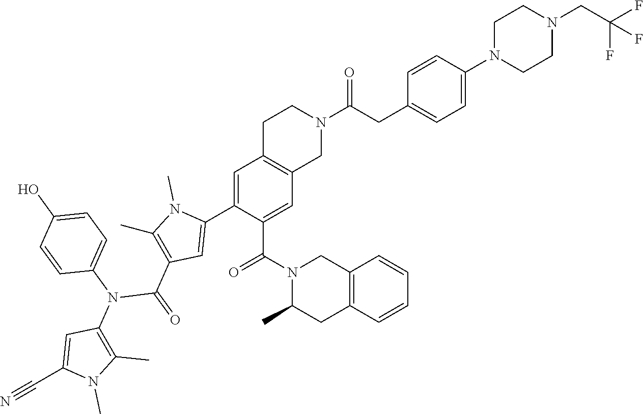 [SMILES: Cc1c(N(C(=O)c2cc(-c3cc4c(cc3C(=O)N3Cc5ccccc5C[C@H]3C)CN(C(=O)Cc3ccc(N5CCN(CC(F)(F)F)CC5)cc3)CC4)n(C)c2C)c2ccc(O)cc2)cc(C#N)n1C]

### Step A: 2-[4-[4-(2,2,2-trifluoroethyl)piperazin-1-yl]phenyl]acetic acid

To a solution of the methyl 2-(4-bromophenyl)acetate (0.20 mL, 1.4 eq.) and 1-(2,2,2-trifluoroethyl)piperazine (0.40 mL, 1.02 mmol) in 1,4-dioxane (5.0 mL) was added Cs₂CO₃ (439 mg, 1.0 eq.) and [2-(2-aminoethyl)phenyl]-chloro-palladium, dicyclohexyl-[2-(2,4,6-triisopropylphenyl)phenyl]phosphane (1:1) (37 mg, 0.04 eq.), and the mixture was stirred until complete conversion was observed. After filtration, the crude product was hydrolysed using the General procedure 8, to give the desired product (314 mg, 74%).

¹H NMR (500 MHz, DMSO-d6) δ ppm 10.05 (brs, 1H), 7.08 (d, 2H), 6.86 (d, 2H), 3.40 (s, 2H), 3.23 (q, 2H), 3.10 (t, 4H), 2.74 (t, 4H); ¹³C NMR (125 MHz, DMSO-d6) δ ppm 173.6, 150.1, 130.3, 126.5, 126.4, 115.9, 57.3, 53.5, 49, 40.7.

HRMS-ESI (m/z) [M+H]⁺ calcd for C₁₄H₁₈F₃N₂O₃: 303.1320, found 303.1315.

### Step B: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[4-(2,2,2-trifluoroethyl)piperazin-1-yl]phenyl}acetyl)-1,2,3,4-tetrahydroisoquinolin-6-yl}-1H-pyrrole-3-carboxamide

Using General procedure 4c, starting from the product of Step A (100 mg, 3.4 eq.) as the appropriate carboxylic acid and Preparation VIIb (75 mg, 0.10 mmol) as the appropriate amine, afforded the title product (62 mg, 68%).

HRMS-ESI (m/z) [M+H]⁺ calcd for C₅₄H₅₆F₃N₈O₄: 937.4371, found 937.4375.

### Example 24: 5-{2-[2-(4-bromo-2-fluorophenyl)acetyl]-7-[(3R)-3-methyl-1,2,3,4-tetrahydroisoquinoline-2-carbonyl]-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

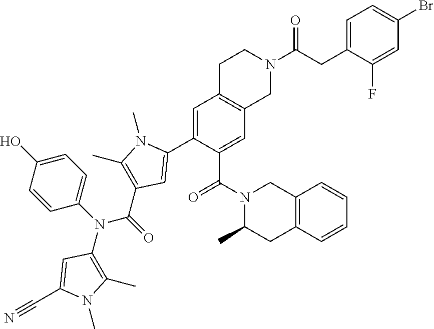 [SMILES: Cc1c(N(C(=O)c2cc(-c3cc4c(cc3C(=O)N3Cc5ccccc5C[C@H]3C)CN(C(=O)Cc3ccc(Br)cc3F)CC4)n(C)c2C)c2ccc(O)cc2)cc(C#N)n1C]

Using General procedure 4c, starting from 2-(4-bromo-2-fluorophenyl)acetic acid (30 mg, 1.3 eq.) as the appropriate carboxylic acid and Preparation VIIb (75 mg, 0.10 mmol, as the appropriate amine, afforded the title compound (41.2 mg, 49%).

HRMS-ESI (m/z) [M+H]⁺ calcd for C₄₈H₄₅BrFN₆O₄: 867.2664, found 867.2661.

### Example 25: 5-{2-benzoyl-7-[(3R)-3-methyl-1,2,3,4-tetrahydroisoquinoline-2-carbonyl]-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

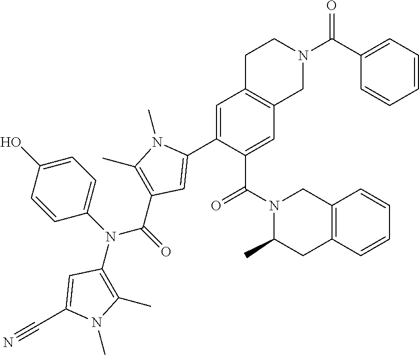 [SMILES: Cc1c(N(C(=O)c2cc(-c3cc4c(cc3C(=O)N3Cc5ccccc5C[C@H]3C)CN(C(=O)c3ccccc3)CC4)n(C)c2C)c2ccc(O)cc2)cc(C#N)n1C]

Using General procedure 4d, starting from benzoyl chloride (30 mg, 1.6 eq.) as the appropriate carboxylic acid and Preparation VIIb (100 mg, 0.13 mmol) as the appropriate amine, afforded the title compound (55 mg, 56%).

HRMS-ESI (m/z) [M+H]⁺ calcd for C₄₇H₄₅N₆O₄: 757.3502, found 757.3494.

### Example 26: N-(5-cyano-1,2-dimethylpyrrol-3-yl)-5-(2-{2,2-difluoro-2-[4-(4-methylpiperazin-1-yl)phenyl]acetyl}-7-{[(3R)-3-methyl-3,4-dihydro-1H-isoquinolin-2-yl]carbonyl}-3,4-dihydro-1H-isoquinolin-6-yl)-N-(4-hydroxyphenyl)-1,2-dimethylpyrrole-3-carboxamide

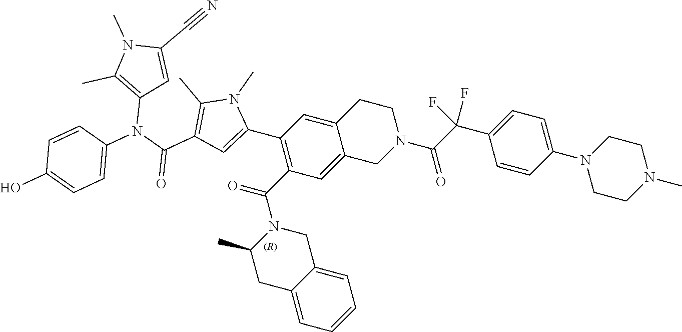

### Step A: ethyl 2-(4-bromophenyl)-2,2-difluoroacetate

Ethyl 2-(4-bromophenyl)-2-oxoacetate (3 g, 11.67 mmol, 1 eq.) was cooled to 0° C. under N₂ followed by the dropwise addition of DAST (3.08 mL, 23.34 mmol, 2 eq.). On complete addition, the mixture was stirred at rt under N₂ for 2 h. The mixture was poured on to ice with stirring and then extracted with EtOAc. The organic phase was washed with brine, dried over MgSO₄ and concentrated in vacuo to afford the title product (3.06 g, 10.95 mmol, 94%).

¹H NMR (400 MHz, DMSO-d6) δ ppm: 7.80-7.73 (m, 2H), 7.58-7.51 (m, 2H), 4.31 (q, J=7.1 Hz, 2H), 1.22 (t, J=7.1 Hz, 3H).

### Step B: difluoro[4-(4-methylpiperazin-1-yl)phenyl]acetic acid

The product from Step A (1 g, 3.58 mmol, 1 eq.), 1-methylpiperazine, (0.6 mL, 5.37 mmol, 1.5 eq.) and Cs₂CO₃ (2.33 g, 7.17 mmol, 2 eq.) were combined in toluene (15 mL) and the mixture sparged with N₂. DavePhos (141 mg, 0.36 mmol, 0.1 eq.) and Pd₂dba₃ (164 mg, 0.18 mmol, 0.05 eq.) were added and the mixture sparged with N₂ and then heated at 120° C. under microwave irradiation for 1 h. The mixture was allowed to cool to rt and filtered through a celite cartridge, washing with EtOAc. The filtrate was washed with sat. aq. NaHCO₃ solution and brine and then dried over MgSO₄ and concentrated in vacuo. Purification by automated flash chromatography eluting with a gradient of 0-100% EtOAc in heptane and then 0-90% MeOH in EtOAc afforded the title product, which was used directly in the subsequent step without further purification (0.86 g, 3.18 mmol, 89%).

LRMS calcd for C₁₃H₁₆F₂N₂O₂: 270.1, found 270.8 [M+H]⁺

¹H NMR (400 MHz, DMSO-d6) δ ppm: 7.36-7.28 (m, 2H), 6.93-6.86 (m, 2H), 3.19-3.11 (m, 4H), 2.47-2.41 (m, 4H), 2.21 (s, 3H).

### Step C: N-(5-cyano-1,2-dimethylpyrrol-3-yl)-5-(2-{2,2-difluoro-2-[4-(4-methylpiperazin-1-yl)phenyl]acetyl}-7-{[(3R)-3-methyl-3,4-dihydro-1H-isoquinolin-2-yl]carbonyl}-3,4-dihydro-1H-isoquinolin-6-yl)-N-(4-hydroxyphenyl)-1,2-dimethylpyrrole-3-carboxamide

Using General procedure 4a and Preparation IVa (200 mg, 0.29 mmol, 1.1 eq.) as the appropriate amine and the product from Step B (102 mg, 0.33 mmol, 1.3 eq.) as the appropriate carboxylic acid afforded the title product (6.7 mg, 0.01 mmol, 3%).

HRMS calcd for C₅₃H₅₄F₂N₈O₄: 904.4236, found 905.4309 [M+H]⁺

### Example 27: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(oxan-4-yl)ethoxy]phenyl}acetyl)-1,2,3,4-tetrahydroisoquinolin-6-yl}-1H-pyrrole-3-carboxamide

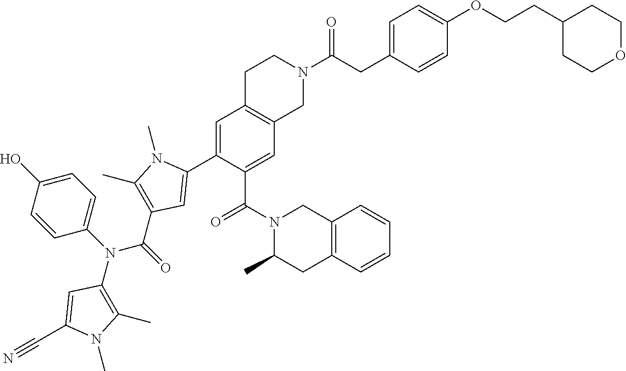 [SMILES: Cc1c(N(C(=O)c2cc(-c3cc4c(cc3C(=O)N3Cc5ccccc5C[C@H]3C)CN(C(=O)Cc3ccc(OCCC5CCOCC5)cc3)CC4)n(C)c2C)c2ccc(O)cc2)cc(C#N)n1C]

Using General procedure 4c, starting from Preparation Vc (49 mg, 1.5 eq.) as the appropriate carboxylic acid and Preparation VIIb (94 mg, 0.12 mmol) as the appropriate amine, afforded the title compound (64 mg, 58%).

HRMS-ESI (m/z) [M+H]⁺ calcd for C₅₅H₅₉N₆O₆: 899.4496, found 899.4498.

### Example 28: N-(5-cyano-1,2-dimethylpyrrol-3-yl)-5-[2-(2-{3-fluoro-4-[1-(2-fluoroethyl)piperidin-4-yl]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

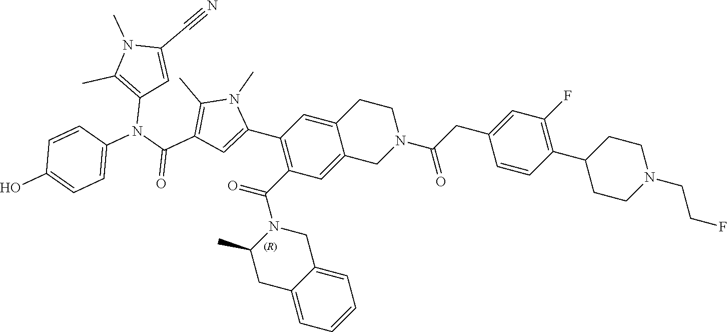

### Step A: tert-butyl 4-[2-fluoro-4-(2-methoxy-2-oxoethyl)phenyl]-3,6-dihydro-2H-pyridine-1-carboxylate

A solution of methyl 2-(4-bromo-3-fluorophenyl)acetate (1.5 g, 6.07 mmol, 1 eq.), 3,6-dihydro-2H-pyridine-1-N-Boc-4-boronic acid, pinacol ester (2.07 g, 6.68 mmol, 1.1 eq.) and K₂CO₃ (2.52 g, 18.21 mmol, 3 eq.) in THE (10 mL) and water (1 mL) was sparged with N₂ followed by the addition of Pd(dppf)Cl₂ DCM (496 mg, 0.61 mmol, 0.1 eq.). The mixture was sparged with N₂ and then heated at 110° C. under microwave irradiation for 1 h. The mixture was allowed to cool to rt, diluted with DCM and washed with water. The phases were separated, and the organic phase was washed with brine, dried over MgSO₄ and concentrated in vacuo. Purification by automated flash chromatography eluting with a gradient of 0-35% EtOAc in heptane afforded the title product (1.52 g, 4.36 mmol, 72%).

LRMS calcd for C₁₉H₂₄FNO₄: 349.2, found 250.0 [M-Boc+H]⁺

¹H NMR (400 MHz, DMSO-d6) δ ppm: 7.29 (t, J=8.1 Hz, 1H), 7.14-7.05 (m, 2H), 6.01-5.92 (m, 1H), 3.98 (d, J=3.4 Hz, 2H), 3.71 (s, 2H), 3.62 (s, 3H), 3.52 (t, J=5.7 Hz, 2H), 2.45-2.37 (m, 2H), 1.43 (s, 9H).

### Step B: tert-butyl 4-[2-fluoro-4-(2-methoxy-2-oxoethyl)phenyl]piperidine-1-carboxylate

To a solution of the product from Step A (1.52 g, 4.36 mmol, 1 eq.) in MeOH (30 mL) was added 10% Pd/C (catalytic). The mixture was evacuated and backfilled with N₂, then evacuated and flushed with H₂ and shaken at rt for 16 h under an atmosphere of H₂. The mixture was filtered through a celite cartridge, washing with MeOH. The solvent was removed in vacuo to afford the title product (1.36 g, 3.88 mmol, 89%).

LRMS calcd for C₁₉H₂₆FNO₄: 351.2, found 252.0 [M+H]⁺

¹H NMR (400 MHz, DMSO-d6) δ ppm: 7.27 (t, J=8.1 Hz, 1H), 7.10-7.01 (m, 2H), 4.12-3.96 (m, 2H), 3.67 (s, 2H), 3.61 (s, 3H), 3.00-2.68 (m, 3H), 1.75-1.64 (m, 2H), 1.60-1.45 (m, 2H), 1.41 (s, 9H).

### Step C: methyl 2-[3-fluoro-4-(piperidin-4-yl)phenyl]acetate hydrochloride

A solution of the product from Step B (682 mg, 1.94 mmol, 1 eq.) in 3 M HCl in MeOH (30 mL) was stirred at rt for 18 h. The solvents were removed in vacuo and then purified by reverse phase automated flash chromatography (Combiflash R_{f}, C18 43 g RediSep column) eluting with a gradient of 0-14% MeCN in water to afford the title product (140 mg, 0.49 mmol, 25%).

LRMS calcd for C₁₄H₁₈FNO₂: 251.1, found 252.0 [M+H]⁺

¹H NMR (400 MHz, DMSO-d6) δ ppm: 8.91 (br s, 2H), 7.13-7.06 (m, 2H), 7.09 (s, 1H), 3.69 (s, 2H), 3.62 (s, 3H), 3.39-3.31 (m, 2H), 3.18-2.95 (m, 3H), 1.98-1.80 (m, 4H).

### Step D: {3-fluoro-4-[1-(2-fluoroethyl)piperidin-4-yl]phenyl}acetic acid

To a solution of the product from Step C (140 mg, 0.49 mmol, 1 eq.) in DMF (5 mL), cooled to 0° C., was added TEA (68 μL, 0.49 mmol, 1 eq.) followed by NaH (60% in mineral oil, 23 mg, 0.58 mmol, 1.2 eq.) and the mixture was stirred for 15 mins. 1-Fluoro-2-iodoethane (44 μL, 0.54 mmol, 1.1 eq.) was added and the mixture was allowed to warm to rt. The reaction was stirred for 18 h. The solvent was removed in vacuo to afford an intermediate which was dissolved in MeOH (10 mL) and treated with 2 M aq. NaOH solution (0.49 mL, 0.97 mmol, 2 eq.). The mixture was heated at 80° C. under microwave irradiation for 0.5 h. The solvent was removed under reduced pressure and the residue diluted with water and the pH was adjusted to 2 using 2 M aq. HCl solution. Purification by reverse phase automated flash chromatography eluting with a gradient of 0-40% MeCN in water afforded the title product (144 mg, 0.46 mmol, 94%).

LRMS calcd for C₁₅H₁₉F₂NO₂: 283.1, found 284.0 [M+H]⁺

### Step E: N-(5-cyano-1,2-dimethylpyrrol-3-yl)-5-[2-(2-{3-fluoro-4-[1-(2-fluoroethyl)piperidin-4-yl]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and Preparation IVa (200 mg, 0.29 mmol, 1 eq.) as the appropriate amine and the product from Step D (107 mg, 0.38 mmol, 1.3 eq.) as the appropriate carboxylic acid afforded the title product (42.5 mg, 0.05 mmol, 16%).

HRMS calcd for C₅₅H₅₇F₂N₇O₄: 917.4440, found 918.4511 [M+H]⁺

### Example 29-a: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-((3R or S)-2-oxo-1-phenylpyrrolidine-3-carbonyl)-1,2,3,4-tetrahydroisoquinolin-6-yl}-1H-pyrrole-3-carboxamide (Diastereoisomer 1)

### Example 29-b: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-((3S or R)-2-oxo-1-phenylpyrrolidine-3-carbonyl)-1,2,3,4-tetrahydroisoquinolin-6-yl}-1H-pyrrole-3-carboxamide (Diastereoisomer 2)

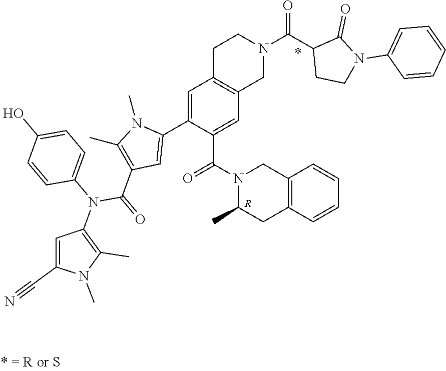

Using General procedure 4c, starting from 2-oxo-1-phenyl-pyrrolidine-3-carboxylic acid (57 mg, 1.5 eq.) as the appropriate carboxylic acid and Preparation VIIb (141 mg, 0.18 mmol) as the appropriate amine, afforded the product as a mixture of diastereoisomers as a white powder (89.4 mg, 58%). The diastereoisomers of the desired product were separated on an AD-3 chiral column (50×500 mm, 20 m) using EtOH as eluent to provide Diastereoisomer 1 (39.5 mg, 26%) and Diastereoisomer 2 (40.1 mg, 27%), the title products.

HRMS-ESI (m/z) [M+H]⁺ calcd for C₅₁H₅₀N₇O₅: 840.3868, found 840.3865 (Diastereosiomer 1)

HRMS-ESI (m/z) [M+H]⁺ calcd for C₅₁H₅₀N₇O₅: 840.3868, found 840.3868 (Diastereosiomer 2)

### Example 30: N-(5-cyano-1,2-dimethyl-1H-pyrrol-3-yl)-N-(4-hydroxyphenyl)-5-[2-(2-{4-[2-(4-hydroxypiperidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-1,2-dimethyl-1H-pyrrole-3-carboxamide

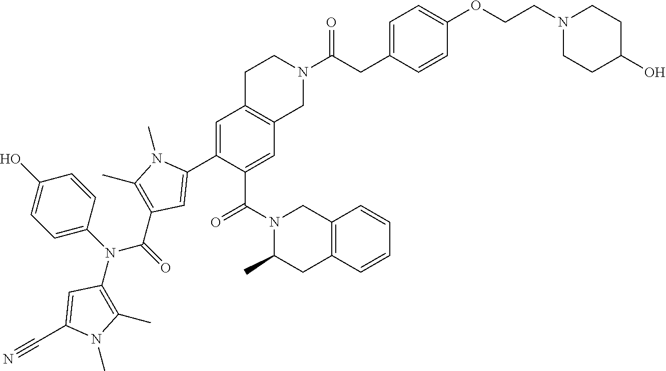 [SMILES: Cc1c(N(C(=O)c2cc(-c3cc4c(cc3C(=O)N3Cc5ccccc5C[C@H]3C)CN(C(=O)Cc3ccc(OCCN5CCC(O)CC5)cc3)CC4)n(C)c2C)c2ccc(O)cc2)cc(C#N)n1C]

### Step A: 1-(2-trityloxymethyl)piperidin-4-ol

To a solution of 1-(2-hydroxyethyl)piperidin-4-ol (1.0 g, 6.887 mmol, 1 eq.) in DCM (10 mL) were added TEA (1.4 mL, 10.0 mmol, 1.5 eq.) and N,N-dimethylpyridin-4-amine (40 mg, 0.327 mmol, 0.05 eq.). The reaction mixture was stirred at rt for 10 min. At 0° C. trityl chloride (2.0 g, 7.174 mmol, 1.04 eq.) was added to the reaction mixture, which was allowed to warm up and was stirred at rt for overnight. The mixture was concentrated in vacuo and the residue was purified by automated flash chromatography using DCM and MeOH as eluents to afford the title product (1.20 g, 45%).

¹H NMR (500 MHz, DMSO-d6) δ ppm 7.44-7.21 (m, 15H), 4.52 (d, 1H), 3.39 (m, 1H), 3.01 (t, 2H), 2.64/2.01 (m+m, 4H), 2.49 (m, 2H), 1.67/1.35 (m+m, 4H); ¹³C NMR (500 MHz, DMSO-d6) δ ppm 144.5, 86.4, 66.7, 62.2, 57.9, 52.1, 35.0.

HRMS-ESI (m/z) [M+H]⁺ calcd for C₂₆H₃₀NO₂: 388.2271, found 388.2278

### Step B: tert-butyl-dimethyl-[[1-(2-trityloxyethyl)-4-piperidyl]oxy]silane

The product of Step A (1.20 g, 3.100 mmol, 1 eq.) in DCM (6.45 mL/mmol) was treated with imidazole (500 mg, 7.344 mmol, 2.4 eq.) and tert-butyl-chloro-dimethyl-silane (860 mg, 5.706 mmol, 1.8 eq.) at 0° C. The reaction mixture was allowed to warm up and stirred at rt overnight. The mixture was concentrated in vacuo and the residue was purified by automated flash chromatography using DCM and MeOH as eluents to afford the title product (1.40 g, 90%).

¹H NMR (400 MHz, DMSO-d6) δ ppm 7.44-7.22 (m, 15H), 3.66 (m, 1H), 3.03 (t, 2H), 2.61 (br, 2H), 2.12 (t, 2H), 1.66 (m, 2H), 1.41 (m, 2H), 0.86 (s, 9H), 0.03 (s, 6H).

HPLC-MS (m/z) [M+H]⁺ calcd for C₃₂H₄₄NO₂Si: 502.3, found 502.2.

### Step C. 2-[4-[tert-butyl(dimethyl)silyl]oxy-1-piperidyl]ethanol

The product of Step B (1.40 g, 2.800 mmol, 1 eq.) in DCM (3.57 mL/mmol) was treated with acetic acid (20 mL, 349.4 mmol, 30 eq.) and stirred at 100° C. for overnight. The mixture was concentrated in vacuo and the residue was dissolved in MeOH, treated with K₂CO₃ (2.0 g, 14.471 mmol, 5.2 eq.) and stirred at rt for 2 h. The mixture was concentrated in vacuo. The crude product was taken up in H₂O (15 mL), extracted with DCM (3×10 mL), the combined organic layers were washed with brine (20 mL), dried over sodium sulphate, filtered and evaporated. The crude product was purified by automated flash chromatography using DCM and MeOH as eluents (Visualization on TLC: KMnO₄) to afford the title product (380 mg, 52%).

¹H NMR (400 MHz, DMSO-d6) δ ppm 4.34 (t, 1H), 3.65 (m, 1H), 3.45 (m, 2H), 2.64 (m, 2H), 2.34 (t, 2H), 2.13 (t, 2H), 1.66 (m, 2H), 1.39 (m, 2H), 0.86 (s, 9H), 0.04 (s, 6H).

HPLC-MS (m/z) [M+H]⁺ calcd for C₁₃H₃₀NO₂Si: 260.2, found 260.2

### Step D: methyl 2-[4-[2-[4-[tert-butyl(dimethyl)silyl]oxy-1-piperidyl]ethoxy]phenyl]acetate

Using General procedure 7b the product of Step C (380 mg, 1.464 mmol, 1.0 eq.), methyl 2-(4-hydroxyphenyl)acetate (330 mg, 1.986 mmol, 1.4 eq.), PPh₃ (660 mg, 2.516 mmol, 1.7 eq.) and DTBAD (580 mg, 2.519 mmol, 1.7 eq.) in THE (8.0 mL) were stirred at rt for 1 h. The reaction mixture was evaporated. To the crude product was added H₂O (15 mL), extracted with DCM (3×10 mL), the combined organic layers were washed with brine (20 mL), dried over sodium sulphate, filtered and evaporated. The crude product was triturated with MeCN. The precipitate was filtered. The mother liquor was concentrated in vacuo and the residue was purified by reverse phase automated flash chromatography using MeCN and water as eluents to afford the title product (140 mg, 23%).

¹H NMR (400 MHz, DMSO-d6) δ ppm 7.15 (m, 2H), 6.87 (m, 2H), 4.01 (t, 2H), 3.69 (m, 1H), 3.60 (s, 3H), 3.59 (s, 2H), 2.72 (m, 2H), 2.66 (t, 2H). 2.23 (t, 2H), 1.70 (m, 2H), 1.42 (m, 2H), 0.86 (s, 9H), 0.04 (s, 6H).

HPLC-MS (m/z) [M+H]⁺ calcd for C₂₂H₃₈NO₄Si: 408.3, found 408.2

### Step E: 2-[4-[2-(4-hydroxy-1-piperidyl)ethoxy]phenyl]acetic acid

The product of Step D (120 mg, 0.294 mmol, 1 eq.) in 1,4-dioxane (2 mL) was treated with 2M aq. NaOH solution (1.0 mL, 2.0 mmol, 6.8 eq.) and stirred at 90° C. for 1 h. The reaction mixture was acidified with 2M aq. HCl solution (few drops) to pH-3 and evaporated. Toluene was added then the residual water was removed by azeotropic distillation. The compound was isolated (it contains inorganic salts) and used as a crude product in the next coupling step (75 mg, 91%).

¹H NMR (400 MHz, DMSO-d6) δ ppm 12.33 (br, 1H), 10.95 (brs, 1H), 7.19 (m, 2H), 6.94 (m, 2H), 4.40 (t, 2H), 3.93 (m, 1H), 3.63 (m, 2H), 3.51 (s, 2H), 3.25 (m, 2H), 3.05 (m, 2H), 2.11-1.67 (m, 4H).

HPLC-MS (m/z) [M+H]⁺ calcd for C₁₅H₂₂NO₄: 280.1, found 280.2

### Step F. N-(5-cyano-1,2-dimethyl-1H-pyrrol-3-yl)-N-(4-hydroxyphenyl)-5-[2-(2-{4-[2-(4-hydroxypiperidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-1,2-dimethyl-1H-pyrrole-3-carboxamide

Using General procedure 4c, starting from the product of Step E (75 mg, 2.2 eq.) as the appropriate carboxylic acid and Preparation VIIb (80 mg, 0.12 mmol) as the appropriate amine, afforded the title compound (15 mg, 13%).

HRMS-ESI (m/z) [M+H]⁺ calcd for C₅₅H₆₀N₇O₆: 914.4605, found 914.4599.

### Example 31: N-(5-cyano-1,2-dimethyl-1H-pyrrol-3-yl)-5-[2-(2-{4-[2-(4-cyclopropylpiperazin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

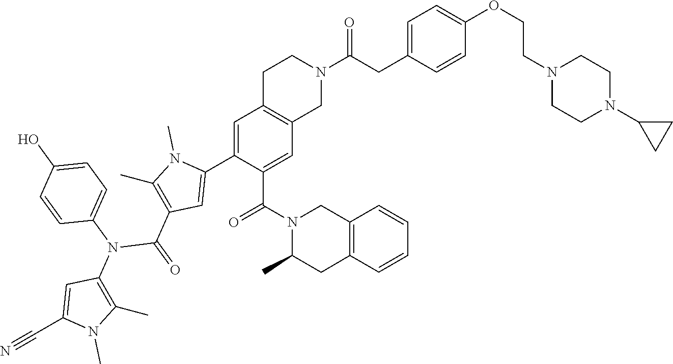 [SMILES: Cc1c(N(C(=O)c2cc(-c3cc4c(cc3C(=O)N3Cc5ccccc5C[C@H]3C)CN(C(=O)Cc3ccc(OCCN5CCN(C6CC6)CC5)cc3)CC4)n(C)c2C)c2ccc(O)cc2)cc(C#N)n1C]

### Step A: 2-[4-[2-(4-cyclopropylpiperazin-1-yl)ethoxy]phenyl]acetic acid

The suspension of 1-cyclopropylpiperazine (164 mg, 1.30 mmol), 2-bromoethanol (0.14 mL, 1.5 eq.), and K₂CO₃ (350 mg, 1.9 eq.) in ethanol (3 mL) were stirred at 100° C. until complete conversion was observed. After the reaction mixture was diluted with MTBE, filtered, and concentrated, the residue was reacted according to General procedure 7b, using methyl 2-(4-hydroxyphenyl)acetate (190 mg, 1.1 eq.) as the appropriate phenol. The mixture was further hydrolysed according to the General procedure 8 to give the title compound (120 mg, 27%).

¹H NMR (500 MHz, DMSO-d6) δ ppm 7.20 (m, 2H), 6.94 (m, 2H), 4.40 (brt, 2H), 3.90-3.00 (brs, 11H), 3.50 (s, 2H), 0.93/0.69 (brs+m, 2H).

HRMS-ESI (m/z) [M+H]⁺ calcd for C₁₇H₂₅N₂O₃: 305.1865, found 305.1860.

### Step B: N-(5-cyano-1,2-dimethyl-1H-pyrrol-3-yl)-5-[2-(2-{4-[2-(4-cyclopropylpiperazin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from the product of Step A (100 mg, 2.8 eq.) as the appropriate carboxylic acid and Preparation VIIb (80 mg, 0.10 mmol) as the appropriate amine, afforded the title compound (15 mg, 15%).

HRMS-ESI (m/z) [M+H]⁺ calcd for C₅₇H₆₃N₈O₅: 939.4916, found 939.4925.

### Example 32: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-[2-(4-{2-[4-(2,2,2-trifluoroethyl)piperazin-1-yl]ethoxy}phenyl)acetyl]-1,2,3,4-tetrahydroisoquinolin-6-yl}-1H-pyrrole-3-carboxamide

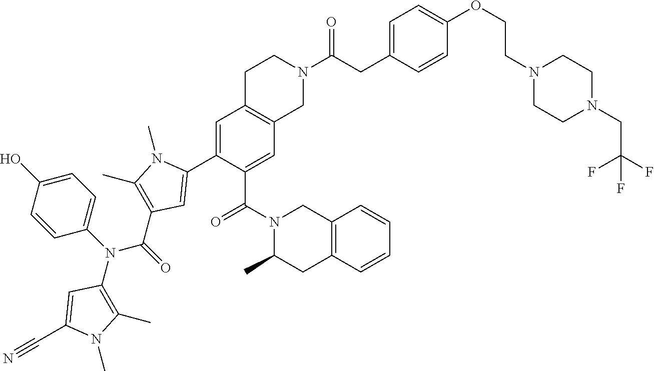 [SMILES: Cc1c(N(C(=O)c2cc(-c3cc4c(cc3C(=O)N3Cc5ccccc5C[C@H]3C)CN(C(=O)Cc3ccc(OCCN5CCN(CC(F)(F)F)CC5)cc3)CC4)n(C)c2C)c2ccc(O)cc2)cc(C#N)n1C]

### Step A: methyl 2-[4-(2-hydroxyethoxy)phenyl]acetate

A suspension of methyl 2-(4-hydroxyphenyl)acetate (1.9 g, 11.0 mmol), 2-bromoethanol (1.2 mL, 1.5 eq), and K₂CO₃ (3 g, 1.9 eq) in DMF (10 mL) was stirred at 100° C. until complete conversion was observed. The reaction mixture was concentrated and the residue was purified by flash chromatography using DCM and MeOH as eluents, to give the title compound (1.2 g, 50%).

¹H NMR (500 MHz, DMSO-d6) δ ppm 7.16 (m, 2H), 6.88 (m, 2H), 3.95 (t, 2H), 3.69 (t, 2H), 3.59 (s, 3H), 3.59 (s, 2); ¹³C NMR (125 MHz, DMSO-d6) δ ppm 172.4, 158.1, 130.8, 126.6, 114.8, 69.9, 60.0, 52.1, 39.7.

HRMS-ESI (m/z) [M+H]⁺ calcd for C₁₁H₁₅O₄: 211.0965, found 211.0965.

### Step B: methyl 2-[4-(2-methylsulfonyloxyethoxy)phenyl]acetate

To the product of Step A (1.18 g, 5.6 mmol) and DIPEA (1.76 mL, 1.8 equiv.) in THE (20 ml) was added methanesulfonyl chloride (0.65 ml, 1.5 eq.) dropwise, and the reaction mixture was stirred until the reaction was completed. After dilution with DCM (50 mL), the organic phase was washed with water (20 mL) and brine (20 ml), dried, and concentrated to give the desired product (1.52 g, 94%).

¹H NMR (500 MHz, DMSO-d6) δ ppm 7.19 (d, 2H), 6.92 (d, 2H), 4.52 (t, 2H), 4.23 (t, 2H), 3.61 (s, 2H), 3.60 (s, 3H), 3.23 (s, 3H); ¹³C NMR (125 MHz, DMSO-d6) δ ppm 172.3, 157.3, 131.0, 127.3, 114.9, 69.2, 66.2, 52.1, 39.7, 37.3.

HRMS-ESI (m/z) [M−H]⁻ calcd for C₁₂H₁₇O₆S: 289.0740, found 289.0743.

### Step C: 2-[4-[2-[4-(2,2,2-trifluoroethyl)piperazin-1-yl]ethoxy]phenyl]acetic acid

To a suspension of]-(2,2,2-trifluoroethyl)piperazine (247 mg, 2.6 eq.) in 1,4-dioxane (3 mL), the product of Step B (164 mg, 0.57 mmol) and DIPEA (0.15 mL, 1.5 eq.) were added. The mixture was stirred at 100° C. until complete conversion was observed, then the product was hydrolysed according to General procedure 8 to give the title compound (150 mg, 76%).

¹H NMR (500 MHz, DMSO-d6) δ ppm 11.83 (brs, 1H), 7.16 (m, 2H), 6.87 (m, 2H), 4.06 (t, 2H), 3.47 (s, 2H), 3.12 (q, 2H), 2.69 (t, 2H), 2.63 (m, 4H), 2.51 (m, 4H); ¹³C NMR (125 MHz, DMSO-d6) δ ppm 173.1, 130.7, 126.5, 115.0, 66.4, 57.6, 57.0, 53.6, 53.4, 40.4.

HRMS-ESI (m/z) [M+H]⁺ calcd for C₁₆H₂₂F₃N₂O₃: 347.1582, found 347.1582.

### Step D: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-[2-(4-{2-[4-(2,2,2-trifluoroethyl)piperazin-1-yl]ethoxy]phenyl)acetyl]-1,2,3,4-tetrahydroisoquinolin-6-yl}-1H-pyrrole-3-carboxamide

Using General procedure 4c, starting from the product of Step C (150 mg, 3.3 eq.) as the appropriate carboxylic acid and Preparation VIIb (100 mg, 0.13 mmol) as the appropriate amine, afforded the title compound (72 mg, 56%).

HRMS-ESI (m/z) [M+H]⁺ calcd for C₅₆H₆₀F₃N₈O₅: 981.4633, found 981.4630.

### Example 33: N-(5-cyano-1,2-dimethylpyrrol-3-yl)-5-{2-[2-(2-fluoro-4-methoxyphenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

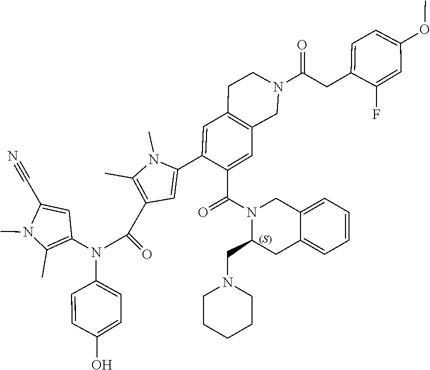

### Step A: methyl 2-(2-fluoro-4-methoxyphenyl)acetate

Using General procedure 7b and Preparation Vb, Step A (940 mg, 5.1 mmol, 1 eq.) as the appropriate phenol and MeOH (4 mL) as the appropriate alcohol afforded the title product (543 mg, 2.74 mmol, 54%).

LRMS calcd for C₁₀H₁₁FO₃: 198.1, found 199.2 [M+H]⁺

¹H NMR (400 MHz, DMSO-d6) δ ppm: 7.27-7.21 (m, 1H), 6.81 (dd, J=12.0, 2.6 Hz, 1H), 6.74 (ddd, J=8.4, 2.6, 0.7 Hz, 1H), 3.75 (s, 3H), 3.65-3.62 (m, 2H), 3.61 (s, 3H).

### Step B: 2-(2-fluoro-4-methoxyphenyl)acetic acid

To a solution of the product from Step A (543 mg, 2.74 mmol, 1 eq.) in MeOH (5 mL) was added LiOH·H₂O (70 mg, 1.66 mmol, 2 eq.) and the mixture was heated at 100° C. under microwave irradiation for 30 mins. The mixture was allowed to cool to rt, diluted with DCM and washed with 2 M aq. HCl solution. The phases were separated and the organic phase dried (PTFE phase separator) and concentrated in vacuo to afford the title product (450 mg, 2.44 mmol, 89%).

LRMS calcd for C₉H₉FO₃: 184.1, found 183.0 [M−H]⁻

¹H NMR (400 MHz, DMSO-d6) δ ppm: 12.35 (s, 1H), 7.25-7.19 (m, 1H), 6.79 (dd, J=12.0, 2.6 Hz, 1H), 6.73 (ddd, J=8.4, 2.6, 0.7 Hz, 1H), 3.75 (s, 3H), 3.54-3.50 (m, 2H).

### Step C: N-(5-cyano-1,2-dimethylpyrrol-3-yl)-5-{2-[2-(2-fluoro-4-methoxyphenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

Using General procedure 4a and Preparation IVb (130 mg, 0.18 mmol, 1 eq.) as the appropriate amine and the product from Step B (36 mg, 0.19 mmol, 1.1 eq.) as the appropriate carboxylic acid afforded the title product (56.7 mg, 0.06 mmol, 36%).

HRMS calcd for C₅₄H₅₆FN₇O₅: 901.4327, found 902.4407 [M+H]⁺

### Example 34: N-(5-cyano-1,2-dimethylpyrrol-3-yl)-5-[2-(2-{2-fluoro-4-[2-(oxan-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

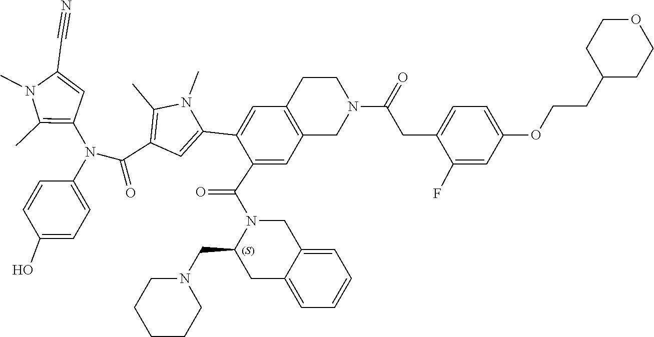

### Step A: methyl 2-{2-fluoro-4-[2-(oxan-4-yl)ethoxy]phenyl}acetate

To a solution of Preparation Vb, Step A (220 mg, 1.19 mmol, 1 eq.) in DMF (3 mL) was added 4-(2-bromoethyl)tetrahydropyran (231 mg, 1.19 mmol, 1 eq.) and K₂CO₃ (330 mg, 2.39 mmol, 2 eq.) and the mixture was stirred at 60° C. under N₂ for 18 h. The mixture was allowed to cool to rt, diluted with DCM and washed with water. The phases were separated, and the organic phase was dried (PTFE phase separator) and concentrated in vacuo. Purification by automated flash chromatography eluting with a gradient of 0-50% EtOAc in heptane afforded the title product (246 mg, 0.83 mmol, 69%).

LRMS calcd for C₁₆H₂₁F₄: 296.1, found 297.2 [M+H]⁺

¹H NMR (400 MHz, DMSO-d6) δ ppm: 7.22 (t, J=8.7 Hz, 1H), 6.81 (dd, J=12.1, 2.5 Hz, 1H), 6.75-6.71 (m, 1H), 4.01 (t, J=6.3 Hz, 2H), 3.86-3.78 (m, 2H), 3.64-3.62 (m, 2H), 3.61 (s, 3H), 3.28 (td, J=11.8, 2.1 Hz, 2H), 1.76-1.56 (m, 5H), 1.28-1.12 (m, 2H).

### Step B: 2-{2-fluoro-4-[2-(oxan-4-yl)ethoxy]phenyl}acetic acid

To a solution of the product from Step A (246 mg, 0.83 mmol, 1 eq.) in MeOH (3 mL) was added LiOH·H₂O (70 mg, 1.66 mmol, 2 eq.) and the mixture was heated at 100° C. under microwave irradiation for 30 mins. The mixture was allowed to cool to rt and partitioned between DCM and 2 M aq. HCl solution. The phases were separated and the organic phase dried (PTFE phase separator) and concentrated in vacuo to afford the title product (209 mg, 0.74 mmol, 89%).

LRMS calcd for C₁₅H₁₉FO₄: 282.1, found 281.2 [M−H]⁻

¹H NMR (400 MHz, DMSO-d6) δ ppm: 12.38 (s, 1H), 7.23-7.16 (m, 1H), 6.79 (dd, J=12.0, 2.5 Hz, 1H), 6.72 (dd, J=8.4, 2.5 Hz, 1H), 4.01 (t, J=6.3 Hz, 2H), 3.86-3.78 (m, 2H), 3.54-3.48 (m, 2H), 3.28 (td, J=11.7, 2.1 Hz, 2H), 1.76-1.56 (m, 5H), 1.28-1.14 (m, 2H).

### Step C: N-(5-cyano-1,2-dimethylpyrrol-3-yl)-5-[2-(2-{2-fluoro-4-[2-(oxan-4-yl)ethoxy]phenyl]acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and Preparation IVb (130 mg, 0.18 mmol, 1 eq.) as the appropriate amine and the product from Step B (55 mg, 0.19 mmol, 1.1 eq.) as the appropriate carboxylic acid afforded the title product (69.5 mg, 0.07 mmol, 39%).

HRMS calcd for C₆₀H₆₆FN₇O₆: 999.5059, found 1000.5136 [M+H]⁺

### Example 35: N-(5-cyano-1,2-dimethyl-pyrrol-3-yl)-3-[2-[2-[4-[2-[cyclopropyl(methyl)amino]ethoxy]phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-5,6,7,8-tetrahydroindolizine-1-carboxamide

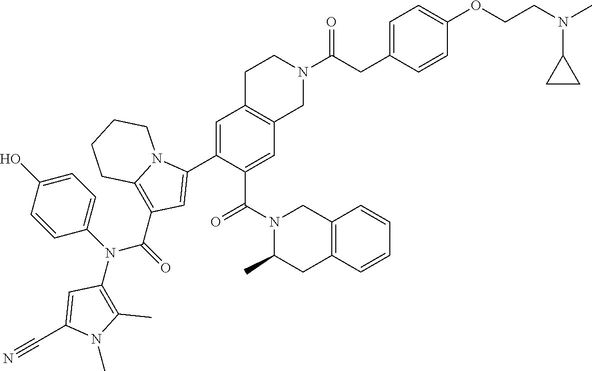 [SMILES: Cc1c(N(C(=O)c2cc(-c3cc4c(cc3C(=O)N3Cc5ccccc5C[C@H]3C)CN(C(=O)Cc3ccc(OCCN(C)C5CC5)cc3)CC4)n3c2CCCC3)c2ccc(O)cc2)cc(C#N)n1C]

### Step A: benzyl 7-formyl-6-(1-methoxycarbonyl-5,6,7,8-tetrahydroindolizin-3-yl)-3,4-dihydro-1H-isoquinoline-2-carboxylate

To benzyl 6-bromo-7-formyl-3,4-dihydro-]H-isoquinoline-2-carboxylate (5.2 g, 14.03 mmol) and methyl 5,6,7,8-tetrahydroindolizine-1-carboxylate (3.8 g, 1.5 eq.) in DMF (18 mL) was added K₂CO₃ (3.9 g, 2.0 eq.) and diacetoxypalladium (315 mg, 0.1 eq.) and the mixture was stirred at 100° C. until complete conversion was observed. The reaction mixture was concentrated and purified by flash chromatography using heptane and EtOAc as eluents, to give the title compound (3.7 g, 56%).

¹H NMR (500 MHz, DMSO-d6) δ ppm 9.78 (s, 1H), 7.76 (s, 1H), 7.44-7.28 (m, 5H), 7.37 (s, 2H), 6.41 (s, 1H), 5.14 (s, 2H), 4.86/4.75 (brs, 2H), 3.69 (s, 3H), 3.68 (brm, 2H), 3.68 (brm, 2H), 3.04 (t, 2H), 2.92 (t, 2H), 1.90-1.74 (m, 4H); ¹³C NMR (125 MHz, DMSO-d6) δ ppm 191.8, 165.0, 138.1, 132.5, 127.3, 126.2, 112.7, 110.2, 66.9, 50.9, 45.6, 44.6, 41.2, 28.9, 24.2.

HRMS-ESI (m/z) [M+H]⁺ calcd for C₂₈H₂₉N₂O₅: 473.2072, found 473.2074.

### Step B: 2-benzyloxycarbonyl-6-(1-methoxycarbonyl-5,6,7,8-tetrahydroindolizin-3-yl)-3,4-dihydro-1H-isoquinoline-7-carboxylic acid

To the product of Step A (3.7 g, 7.87 mmol), 2-methylbut-2-ene (6.3 mL, 7.5 eq.) in 2-methylpropan-2-ol (36 mL) and THE (24 mL) was added NaH₂PO₄ (4.7 g, 5.0 eq.) in water (31 mL) dropwise. After cooling to 14° C. and dropwise addition of NaClO₂ (2.1 g, 3.0 eq.) in water (31 mL), the mixture was stirred at 20° C. until complete conversion was observed. After cooling to 5-10° C., dropwise addition of Na₂S₂O₃ (1.6 g, 1.3 eq.) in water (24 mL), and dilution with MTBE (150 mL), the organic phase was washed with brine (150 mL), dried, concentrated, and purified by flash chromatography using heptane and EtOAc as eluents, to give the title compound (2.5 g, 64%).

¹H NMR (500 MHz, DMSO-d6) δ ppm 12.71 (s, 1H), 7.69 (s, 1H), 7.45-7.28 (m, 5H), 7.18 (s, 1H), 6.22 (s, 1H), 5.14 (s, 2H), 4.70/4.64 (brs/brs., 2H), 3.67 (s, 3H), 3.67 (br., 2H), 3.54 (t, 2H), 3.00 (t, 2H), 2.86 (t, 2H), 1.85-1.67 (m, 4H); ¹³C NMR (125 MHz, DMSO-d6) δ ppm 168.6, 165.2, 155.1, 137.4, 136.4, 134.0, 133.1, 131.5, 131.3, 128.1, 109.6, 108.8, 66.8, 50.8, 45.5, 44.1, 41.3, 28.4, 24.1, 22.9/19.9.

HRMS-ESI (m/z) [M+H]⁺ calcd for C₂₈H₂₉N₂O₆: 489.2021, found 489.2023.

### Step C: 3-[7-[(3R)-3-methyl-3,4-dihydro-1H-isoquinoline-2-carbonyl]-1,2,3,4-tetrahydroisoquinolin-6-yl]-5,6,7,8-tetrahydroindolizine-1-carboxylic acid

Using the procedure described in Step A of Preparation VIIb, starting from the product of Step B (3.1 g, 6.45 mmol) as the appropriate carboxylic acid and the product from Step A of Preparation Ha (1.4 mg, 1.2 eq.) as the appropriate amine, the title compound was obtained (3.1 g, 83%).

¹H NMR (500 MHz, DMSO-d6) δ ppm 9.85 (brs, 2H), 7.36-6.87 (m, 6H), 6.39-6.18 (s, 1H), 5.32-1.33 (m, 19H), 1.04-0.54 (d, 3H).

HRMS-ESI (m/z) [M+H]⁺ calcd for C₂₉H₃₂N₃O₃: 470.2437, found 470.2439.

### Step D: 3-[2-(9H-fluoren-9-ylmethoxycarbonyl)-7-[(3R)-3-methyl-3,4-dihydro-1H-isoquinoline-2-carbonyl]-3,4-dihydro-1H-isoquinolin-6-yl]-5,6,7,8-tetrahydroindolizine-1-carboxylic acid

To the product of Step C (3.1 g, 5.3 mmol), NaHCO₃ (1.5 g, 2.3 eq.) in 1,4-dioxane (30 mL) and water (30 mL) was added 9H-fluoren-9-ylmethyl carbonochloridate (208 mg, 1.1 eq.), and the reaction mixture was stirred until complete conversion was observed. After the addition of a 2 M solution of HCl (53 mL), stirring for 15 min, and dilution with DCM (150 mL), the organic phase was washed with brine (150 ml), dried, concentrated, and purified by flash chromatography using DCM and MeOH as eluents to give the title compound (3.0 g, 82%).

¹H NMR (400 MHz, DMSO-d6) δ ppm 8.01-6.85 (m, 14H), 6.44-6.14 (br., 1H), 5.42-3.31 (br., 9H), 4.53-4.29 (br., 2H), 4.32 (br., 1H), 3.14-1.27 (br., 10H), 1.04/0.86/0.75/0.54 (br/br/br/d, 3H); ¹³C NMR (100 MHz, DMSO-d6) δ ppm 111.2, 67.2, 47.3, 18.6/16.9/16.7/16.3.

HRMS-ESI (m/z) [M+H]⁺ calcd for C₄₄H₄₂N₃O₅: 692.3119, found 692.3118.

### Step E: 9H-fluoren-9-ylmethyl 6-[1-[[4-[tert-butyl(dimethyl)silyl]oxyphenyl]-(5-cyano-1,2-dimethyl-pyrrol-3-yl)carbamoyl]-5,6,7,8-tetrahydroindolizin-3-yl]-7-[(3R)-3-methyl-3,4-dihydro-1H-isoquinoline-2-carbonyl]-3,4-dihydro-1H-isoquinoline-2-carboxylate

Using General procedure 2b, starting from the product of Step D (2.5 g, 3.6 mmol) as the appropriate acid and 4-[4-[tert-butyl(dimethyl)silyl]oxyanilino]-1,5-dimethyl-pyrrole-2-carbonitrile (1.5 g, 1.2 eq.) as the appropriate aniline, the title compound was obtained (2.3 g, 62%).

¹H NMR (400 MHz, DMSO-d6) δ ppm 7.93-6.56 (m, 19H), 5.41-5.15 (s, 1H), 5.03-1.42 (m, 22H), 3.67-3.54 (s, 3H), 2.16-1.81 (s, 3H), 1.11-0.45 (d, 3H), 0.90-0.83 (s, 9H), 0.14-0.08 (s, 6H).

HRMS-ESI (m/z) [M+H]⁺ calcd for C₆₃H₆₇N₆O₅Si: 1015.4939, found 1015.4940.

### Step F. N-[4-[tert-butyl(dimethyl)silyl]oxyphenyl]-N-(5-cyano-1,2-dimethyl-pyrrol-3-yl)-3-[7-[(3R)-3-methyl-3,4-dihydro-1H-isoquinoline-2-carbonyl]-1,2,3,4-tetrahydroisoquinolin-6-yl]-5,6,7,8-tetrahydroindolizine-1-carboxamide

The product of Step E (2.26 g, 2.23 mmol) in morpholine (11 mL) and DCM (11 mL) were stirred until complete conversion was observed. The reaction mixture was concentrated and purified by flash chromatography using DCM and MeOH as eluents, to give the title compound (1.7 g, 97%).

¹H NMR (400 MHz, DMSO-d6) δ ppm 7.31-6.53 (m, 11H), 5.41-4.98 (s, 1H), 5.38-1.20 (m, 20H), 3.71-3.51 (s, 3H), 2.18-1.77 (s, 3H), 1.07-0.43 (d, 3H), 0.92-0.87 (s, 9H), 0.16-0.10 (s, 6H).

HRMS-ESI (m/z) [M+H]⁺ calcd for C₄₈H₅₇N₆O₃Si: 793.4255, found 793.4256.

### Step G: 2-[4-[2-[cyclopropyl(methyl)amino]ethoxy]phenyl]acetic acid

Using the General procedures 7d and then General procedure 8, starting from methyl 2-[4-(2-bromoethoxy)phenyl]acetate (250 mg, 0.91 mmol) as the appropriate alkyl halide and N-methylcyclopropanamine (130 mg, 2.0 eq.) as the appropriate amine, the desired product was obtained (190 mg, 59%).

¹H NMR (500 MHz, DMSO-d6) δ ppm 11.85 (brs, 1H), 7.10 (dd, 1H), 7.09 (t, 1H), 6.99 (dm, 1H), 4.11 (t, 2H), 3.51 (s, 2H), 2.87 (t, 2H), 2.34 (s, 3H), 1.75 (m, 1H), 0.42/0.30 (m+m, 4H); ¹³C NMR (125 MHz, DMSO-d6) δ ppm 126.0, 117.5, 115.1, 67.2, 56.4, 43.3, 39.9, 38.7, 7.1.

HRMS-ESI (m/z) [M+H]⁺ calcd for C₁₄H₂₀NO₃: 250.1438, found 250.1437.

### Step H: N-(5-cyano-1,2-dimethyl-pyrrol-3-yl)-3-[2-[2-[4-[2-[cyclopropyl(methyl)amino]ethoxy]phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-5,6,7,8-tetrahydroindolizine-1-carboxamide

Using General procedure 4c, starting from the product of Step G (65 mg, 2.0 eq.) as the appropriate carboxylic acid and the product of Step F (100 mg, 0.13 mmol) as the appropriate amine, the title compound was obtained (58 mg, 50%).

HRMS-ESI (m/z) [M+H]⁺ calcd for C₅₆H₆₀N₇O₅: 910.4652, found 910.4653.

### Example 36: 5-{2-[2-(4-{2-[(9aS)-octahydropyrazino[2,1-c][1,4]oxazin-8-yl]ethoxy}phenyl)acetyl]-7-[(3R)-3-methyl-1,2,3,4-tetrahydroisoquinoline-2-carbonyl]-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

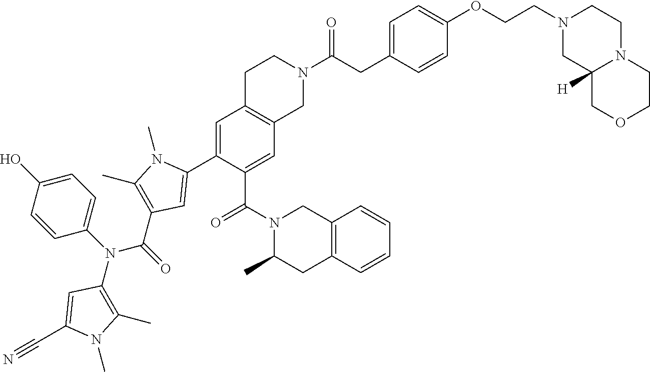 [SMILES: Cc1c(N(C(=O)c2cc(-c3cc4c(cc3C(=O)N3Cc5ccccc5C[C@H]3C)CN(C(=O)Cc3ccc(OCCN5CCN6CCOC[C@@H]6C5)cc3)CC4)n(C)c2C)c2ccc(O)cc2)cc(C#N)n1C]

### Step A: 2-[4-[2-[(9aS)-3,4,6,7,9,9a-hexahydro-1H-pyrazino[2,1-c][1,4]oxazin-8-yl]ethoxy]phenyl]acetic acid

Using the General procedure 7d and then General procedure 8, starting from methyl 2-[4-(2-bromoethoxy)phenyl]acetate (317 mg, 1.16 mmol) as the appropriate alkyl halide and (9aS)-1,3,4,6,7,8,9,9a-octahydropyrazino[2,1-c][1,4]oxazine dihydrochloride (250 mg, 1.0 eq.) as the appropriate amine, the desired product was obtained (90 mg, 25%).

¹H NMR (500 MHz, DMSO-d6) δ ppm 7.12 (dm, 2H), 6.82 (dm, 2H), 4.01 (t, 2H), 3.71/3.47 (brd+m, 2H), 3.57/3.05 (m+m, 2H), 3.35 (s, 2H), 2.84/2.19 (d+m, 2H), 2.69/1.73 (dm+t, 2H), 2.65 (t, 2H), 2.63/2.18 (m+m, 2H), 2.57/2.15 (m+m, 2H), 2.09 (m, 1H); ¹³C NMR (125 MHz, DMSO-d6) δ ppm 173.8, 157.2, 130.7, 129.3, 114.5, 68.9, 66.8, 65.8, 60.0, 57.2, 54.5, 54.1, 54.0, 53.4, 42.1.

HRMS-ESI (m/z) [M+H]⁺ calcd for C₁₇H₂₅N₂O₄: 321.1814, found 321.1807.

### Step B: 5-{2-[2-(4-{2-[(9aS)-octahydropyrazino[2,1-c][1,4]oxazin-8-yl]ethoxy]phenyl)acetyl]-7-[(3R)-3-methyl-1,2,3,4-tetrahydroisoquinoline-2-carbonyl]-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

Using General procedure 4c, starting from the product of Step A (150 mg, 4.0 eq.) as the appropriate carboxylic acid and Preparation VIIb (90 mg, 0.12 mmol) as the appropriate amine, afforded the title compound (54 mg, 48%).

HRMS-ESI (m/z) [M+H]⁺ calcd for C₅₇H₆₃N₈O₆: 955.4865, found 955.4858.

### Example 37: N-(5-cyano-1,2-dimethyl-1H-pyrrol-3-yl)-5-(2-{2-[4-(2-{1,1-dioxo-1λ⁶-thia-6-azaspiro[3.3]heptan-6-yl}ethoxy)phenyl]acetyl}-7-[(3R)-3-methyl-1,2,3,4-tetrahydroisoquinoline-2-carbonyl]-1,2,3,4-tetrahydroisoquinolin-6-yl)-N-(4-hydroxyphenyl)-1,2-dimethyl-1H-pyrrole-3-carboxamide

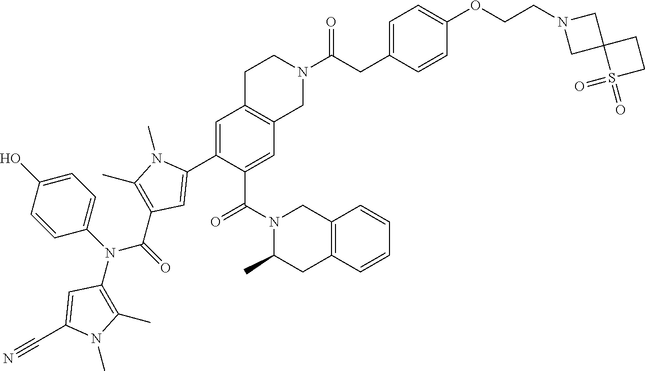 [SMILES: Cc1c(N(C(=O)c2cc(-c3cc4c(cc3C(=O)N3Cc5ccccc5C[C@H]3C)CN(C(=O)Cc3ccc(OCCN5CC6(CCS6(=O)=O)C5)cc3)CC4)n(C)c2C)c2ccc(O)cc2)cc(C#N)n1C]

### Step A: 2-[4-[2-(1,1-dioxo-1λ\hat{ }{6}-thia-6-azaspiro[3.3]heptan-6-yl)ethoxy]phenyl]acetic acid

To the suspension of 3λ\hat{ }{6}-thia-6-azaspiro[3.3]heptane 3,3-dioxide, HCl salt (213 mg, 1.7 eq.) in 1,4-dioxane (1 mL), the product of Step B of Example 32 (200 mg, 0.69 mmol) and DIPEA (0.4 mL, 3.0 eq.) were added. The reaction mixture was stirred at 80° C. until complete conversion was observed, then the product was hydrolysed according to the General procedure 8, to give the title compound (149 mg, 66%).

¹H NMR (500 MHz, DMSO-d6) δ ppm 11.84 (brs, 1H), 7.15 (d, 2H), 6.84 (d, 2H), 3.99 (t, 2H), 3.92 (t, 2H), 3.82/3.34 (brm+brm, 4H), 3.50 (brs, 2H), 2.76 (brm, 2H), 2.21 (t, 2H); ¹³C NMR (125 MHz, DMSO-d6) δ ppm 172.4, 157.5, 130.9, 127.7, 114.6, 78.8, 67.0, 62.4, 60.7, 57.0, 40.5, 17.7.

HRMS-ESI (m/z) [M+H]⁺ calcd for C₁₅H₂₀NO₅S: 326.1062, found 326.1054.

### Step B: N-(5-cyano-1,2-dimethyl-1H-pyrrol-3-yl)-5-(2-{2-[4-(2-{1,1-dioxo-12,6-thia-6-azaspiro[3.3]heptan-6-yl}ethoxy)phenyl]acetyl}-7-[(3R)-3-methyl-1,2,3,4-tetrahydroisoquinoline-2-carbonyl]-1,2,3,4-tetrahydroisoquinolin-6-yl)-N-(4-hydroxyphenyl)-1,2-dimethyl-1H-pyrrole-3-carboxamide

Using General procedure 4c, starting from the product of Step A (120 mg, 3.1 eq.) as the appropriate carboxylic acid and Preparation VIIb (90 mg, 0.12 mmol) as the appropriate amine, afforded the title compound (24 mg, 21%).

HRMS-ESI (m/z) [M+H]⁺ calcd for C₅₅H₅₈N₇O₇S: 960.4113, found 960.4110.

### Example 38: N-(5-cyano-1,2-dimethylpyrrol-3-yl)-N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethyl]phenyl}acetyl)-3,4-dihydro-1H-isoquinolin-6-yl)pyrrole-3-carboxamide

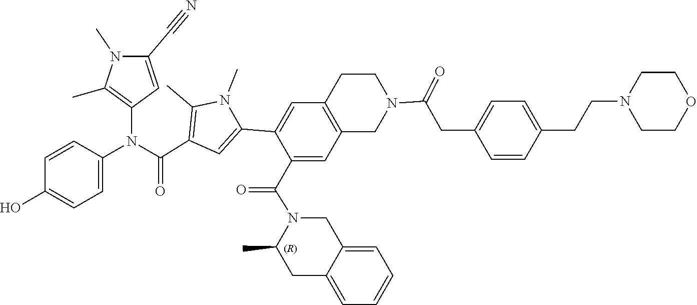

### Step A: methyl 2-{4-[(E)-2-ethoxyethenyl]phenyl}acetate

To a solution of methyl 4-bromophenylacetate (224 mg, 0.98 mmol, 1 eq.), (E)-1-ethoxyethene-2-boronic acid pinacol ester (213 mg, 1.08 mmol, 1.1 eq.) in THE (4 mL) and water (0.4 mL) was added K₂CO₃ (405 mg, 2.93 mmol, 3 eq.), and the mixture was sparged with N₂. Pd(dppf)Cl₂ DCM (80 mg, 0.1 mmol, 0.1 eq.) was added and then the reaction was heated at 110° C. under microwave irradiation for 1 h. The mixture was allowed to cool to rt and partitioned between EtOAc and water. The phases were separated, and the organic phase was dried over MgSO₄ and concentrated in vacuo. Purification by automated flash chromatography eluting with a gradient of 0-36% EtOAc in heptane afforded the title product (173 mg, 0.79 mmol, 80%).

¹H NMR (400 MHz, DMSO-d6) δ ppm: 7.24-7.16 (m, 3H), 7.15-7.10 (m, 2H), 5.80 (d, J=13.0 Hz, 1H), 3.88 (q, J=7.0 Hz, 2H), 3.63-3.58 (m, 5H), 1.25 (t, J=7.0 Hz, 3H).

### Step B: 2-{4-[2-(morpholin-4-yl)ethyl]phenyl}acetic acid

To a solution of the product from Step A (173 mg, 0.79 mmol, 1 eq.) in acetone (5 mL) was added 2 M aq. HCl solution (0.79 mL, 1.57 mmol, 2 eq.) and the mixture was stirred at 45° C. for 2 h. The mixture was allowed to cool to rt, diluted with DCM, dried (PTFE phase separator) and concentrated in vacuo to afford an intermediate which was dissolved in DCM (5 mL). Morpholine (82 μL, 0.94 mmol, 1.2 eq.) and NaBH(OAc)₃ (331 mg, 1.56 mmol, 2 eq.) were added and the mixture was stirred at rt for 2 h. A mixture of ester and acid was observed. Purification by automated flash chromatography eluting with a gradient of 0-20% MeOH in DCM afforded the ester, and then the acid was eluted with MeOH, and the solvent removed in vacuo. The residue was dissolved in DCM and loaded onto a pre-wetted (DCM) SCX-2 cartridge, washing with DCM, MeOH and eluting with 10% NH₃/MeOH in DCM to afford the title product (54 mg, 0.22 mmol, 28%).

¹H NMR (400 MHz, DMSO-d6) δ ppm: 7.13 (s, 4H), 3.59-3.54 (m, 4H), 3.45 (s, 2H), 2.73-2.65 (m, 2H), 2.50-2.45 (m, 2H), 2.44-2.68 (m, 4H).

### Step C: N-(5-cyano-1,2-dimethylpyrrol-3-yl)-N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethyl]phenyl}acetyl)-3,4-dihydro-1H-isoquinolin-6-yl)pyrrole-3-carboxamide

Using General procedure 4a and Preparation IVa (130 mg, 0.2 mmol, 1 eq.) as the appropriate amine and the product from Step B (65 mg, 0.26 mmol, 1.3 eq.) as the appropriate carboxylic acid afforded the title product (67 mg, 0.08 mmol, 38%).

HRMS calcd for C₅₄H₅₇N₇O₅: 883.4421, found 884.4513 [M+H]⁺

### Example 39: N-(5-cyano-1,2-dimethyl-1H-pyrrol-3-yl)-5-{2-[2-(4-{2-[cis-2,6-dimethylmorpholin-4-yl]ethoxy}phenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

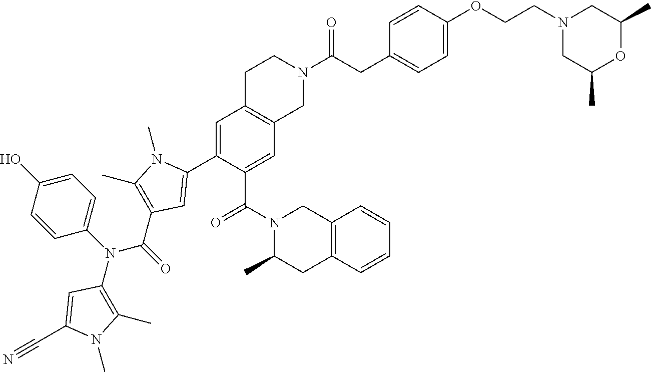 [SMILES: Cc1c(N(C(=O)c2cc(-c3cc4c(cc3C(=O)N3Cc5ccccc5C[C@H]3C)CN(C(=O)Cc3ccc(OCCN5C[C@@H](C)O[C@@H](C)C5)cc3)CC4)n(C)c2C)c2ccc(O)cc2)cc(C#N)n1C]

### Step A: methyl 2-[4-[2-(cis-2,6-dimethylmorpholin-4-yl)ethoxy]phenyl]acetate, methyl 2-[4-[2-((2R,6R)-2,6-dimethylmorpholin-4-yl)ethoxy]phenyl]acetate and methyl 2-[4-[2-((2S,6S)-2,6-dimethylmorpholin-4-yl)ethoxy]phenyl]acetate (mixture of diastereomers)

Using the General procedure 7d, starting from methyl 2-[4-(2-bromoethoxy)phenyl]acetate (1.4 g, 5.10 mmol) as the appropriate alkyl halide and 2,6-dimethylmorpholine (1.8 g, 3.0 eq.) as the appropriate amine, the desired product was obtained (1.4 g, 90%). ¹H NMR (500 MHz, DMSO-d6) δ ppm 7.15 (dm, 2H), 6.88 (dm, 2H), 4.04 (t, 2H), 3.88/3.54 (m/m, 2H), 3.59 (s, 3H), 2.81/2.49/2.17/1.70 (m+dd/m+dd, 4H), 2.65 (t, 2H), 2.59 (s, 2H), 1.11/1.03 (d/d, 6H); ¹³C NMR (500 MHz, DMSO-d6) δ ppm 172.4, 157.9/157.8, 130.8, 126.7, 114.9/114.8, 71.4/66.2, 65.8/65.6, 59.8/59.2, 57.4/57.1, 52.1, 39.7, 19.5/18.6.

HRMS-ESI (m/z) [M+H]⁺ calcd for C₁₇H₂₆NO₄: 308.1856, found 308.1860.

Diastereomers of the desired product was separated on a Daicel AS-V chiral column (100*500 mm, 20 m) using 7:93 i-PrOH-heptane+0.05% DEA as eluents to give the Diastereomer 1, Diastereomer 2 Enantiomer 1 (99.9% ee) and Diastereomer 2 Enantiomer 2 (99.9% ee).

### Step B: 2-[4-[2-[cis-2,6-dimethylmorpholin-4-yl]ethoxy]phenyl]acetic acid (from Diastereomer 1)

Using the General procedure 8, starting from the Diastereomer 1 of Step A (420 mg, 1.37 mmol), the desired product was obtained (360 mg, 67%).

¹H NMR (500 MHz, DMSO-d6) δ ppm 7.14 (d, 2H), 6.85 (d, 2H), 4.04 (t, 2H), 3.55 (m, 2H), 3.42 (s, 2H), 2.81/1.71 (dt+dd, 4H), 2.65 (t, 2H), 1.03 (d, 6H); ¹³C NMR (125 MHz, DMSO-d6) δ ppm 173.8, 157.6, 130.8, 128.2, 114.6, 71.4, 65.6, 59.8, 57.1, 41.0, 19.5.

HRMS-ESI (m/z) [M+H]⁺ calcd for C₁₆H₂₄NO₄: 294.1705, found 294.1702.

### Step C. N-(5-cyano-1,2-dimethyl-1H-pyrrol-3-yl)-5-{2-[2-(4-{2-[cis-2,6-dimethylmorpholin-4-yl]ethoxy]phenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step B (141 mg, 4.8 eq.) as the appropriate carboxylic acid and Preparation VIIb (80 mg, 0.10 mmol) as the appropriate amine, afforded the title compound (60 mg, 62%).

HRMS-ESI (m/z) [M+H]⁺ calcd for C₅₆H₆₂N₇O₆: 928.4761, found 928.4755.

### Example 40: 5-[2-(2-{4-[(8aS)-7,7-difluoro-hexahydropyrrolo[1,2-a]piperazin-2-yl]-2-fluorophenyl}acetyl)-7-{[(3R)-3-methyl-3,4-dihydro-1H-isoquinolin-2-yl]carbonyl}-3,4-dihydro-1H-isoquinolin-6-yl]-N-(5-cyano-1,2-dimethylpyrrol-3-yl)-N-(4-hydroxyphenyl)-1,2-dimethylpyrrole-3-carboxamide

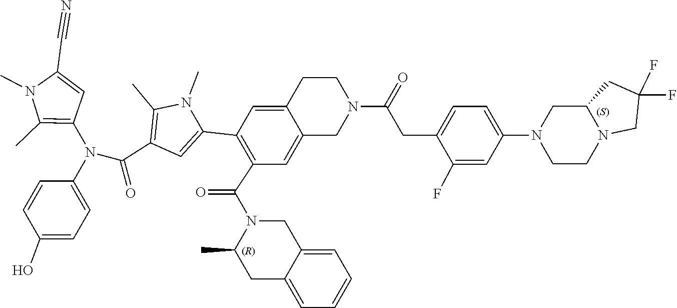

### Step A: methyl 2-{4-[(8aS)-7,7-difluoro-hexahydropyrrolo[1,2-a]piperazin-2-yl]-2-fluorophenyl}acetate

Methyl 2-(4-bromo-2-fluorophenyl)acetate (0.3 mL, 1.82 mmol, 1 eq.), (8aS)-7,7-difluoro-octahydropyrrolo[1,2-a]pyrazine dihydrochloride (535 mg, 2.28 mmol, 1.25 eq.) and Cs₂CO₃ (2.37 g, 7.29 mmol, 4 eq.) were combined in toluene (10 mL) and the mixture was sparged with N₂. DavePhos (72 mg, 0.18 mmol, 0.1 eq.) and Pd₂dba₃ (83 mg, 0.09 mmol, 0.05 eq.) were added and the mixture was heated at 130° C. under microwave irradiation for 1 h. Further DavePhos (36 mg, 0.09 mmol, 0.05 eq.) and Pd₂dba₃ (42 mg, 0.045 mmol, 0.025 eq.) were added, the mixture sparged with N₂ and then heated at 130° C. under microwave irradiation for 2 h. The mixture was allowed to cool to rt and partitioned between DCM and sat. aq. NaHCO₃ solution. The phases were separated (PTFE phase separator) and the organic phase concentrated in vacuo. Purification by automated flash chromatography eluting with a gradient of 0-65% EtOAc in heptane afforded the title product (181 mg, 0.55 mmol, 30%).

LRMS calcd for C₁₆H₁₉F₃N₂O₂: 328.1, found 329.2 [M+H]⁺

¹H NMR (400 MHz, DMSO-d6) δ ppm: 7.16-7.10 (m, 1H), 6.79-6.69 (m, 2H), 3.86-3.76 (m, 1H), 3.71-3.64 (m, 1H), 3.60 (s, 3H), 3.59-3.57 (m, 2H), 3.47-3.37 (m, 1H), 3.04-2.96 (m, 1H), 2.79 (td, J=11.8, 3.3 Hz, 1H), 2.63-2.28 (m, 5H), 2.06-1.87 (m, 1H).

### Step B: {4-[(8aS)-7,7-difluoro-hexahydropyrrolo[1,2-a]piperazin-2-yl]-2-fluorophenyl}acetic acid

To a solution of the product from Step A (181 mg, 0.55 mmol, 1 eq.) in MeOH (4 mL) was added LiOH·H₂O (46 mg, 1.1 mmol, 2 eq.) and the mixture was heated at 100° C. under microwave irradiation for 30 mins. The mixture was allowed to cool to rt, neutralised with 2 M aq. HCl solution and concentrated in vacuo. Purification by reverse phase automated flash chromatography eluting with a gradient of 10-95% MeCN in water afforded the title product (124 mg, 0.39 mmol, 72%).

LRMS calcd for C₁₅H₁₇F₃N₂O₂: 314.1, found 315.2 [M+H]⁺

¹H NMR (400 MHz, DMSO-d6) δ ppm: 7.14-7.05 (m, 1H), 6.74-6.64 (m, 2H), 3.83-3.73 (m, 1H), 3.69-3.61 (m, 1H), 3.48-3.33 (m, 3H), 3.03-2.96 (m, 1H), 2.77 (td, J=11.8, 3.3 Hz, 1H), 2.63-2.45 (m, 3H), 2.43-2.28 (m, 2H), 2.06-1.86 (m, 1H).

### Step C.: 5-[2-(2-{4-[(8aS)-7,7-difluoro-hexahydropyrrolo[1,2-a]piperazin-2-yl]-2-fluorophenyl}acetyl)-7-{[(3R)-3-methyl-3,4-dihydro-1H-isoquinolin-2-yl]carbonyl}-3,4-dihydro-1H-isoquinolin-6-yl]-N-(5-cyano-1,2-dimethylpyrrol-3-yl)-N-(4-hydroxyphenyl)-1,2-dimethylpyrrole-3-carboxamide

Using General procedure 4a and Preparation IVa (130 mg, 0.2 mmol, 1 eq.) as the appropriate amine and the product from Step B (69 mg, 0.22 mmol, 1.1 eq.) as the appropriate carboxylic acid afforded the title product (80.3 mg, 0.08 mmol, 42%).

HRMS calcd for C₅₅H₅₅F₃N₈O₄: 948.4298, found 949.4376 [M+H]⁺

### Example 41: N-(5-cyano-1-ethyl-2-methyl-pyrrol-3-yl)-N-(4-hydroxyphenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

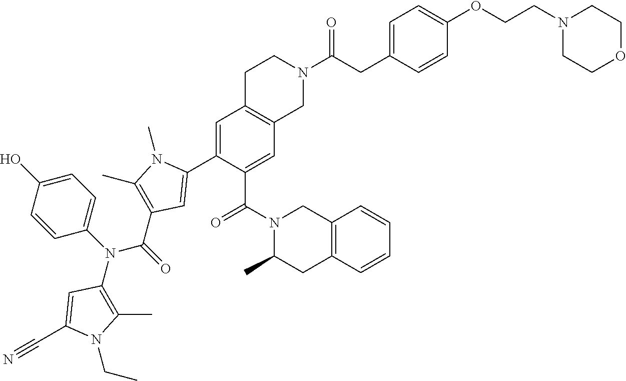 [SMILES: CCn1c(C#N)cc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(O)cc2)c1C]

### Step A: 4-bromo-1-ethyl-5-methyl-pyrrole-2-carbonitrile

Using General procedure 7c, starting from 4-bromo-5-methyl-1H-pyrrole-2-carbonitrile (500 mg, 2.7 mmol.) and ethanol (0.3 ml, 1.9 eq) as the appropriate alcohol, afforded the title product (500 mg, 87%).

GC-MS (m/z) [M+H]⁺ calcd for C₈H₁₀BrN₂: 213, found 212/215.

### Step B: 4-[4-[tert-butyl(dimethyl)silyl]oxyanilino]-1-ethyl-5-methyl-pyrrole-2-carbonitrile

Using General procedure 1b, starting from the product of Step A (400 mg, 1.87 mmol) as the appropriate aryl bromide and Preparation IIIa (629 mg, 1.5 eq.) as the appropriate amine, afforded the title product (570 mg, 85%).

¹H NMR (500 MHz, DMSO-d6) δ ppm 6.87 (s, 1H), 6.75 (s, 1H), 6.60 (m, 2H), 6.50 (m, 2H), 4.02 (q, 2H), 1.28 (t, 3H), 0.92 (s, 9H), 0.12 (s, 6H); ¹³C NMR (125 MHz, DMSO-d6) δ ppm 129.9, 124.9, 120.6, 115.7, 114.9, 114.2, 98.7, 41.3, 26.1, 16.5, −4.1.

HRMS-ESI (m/z) [M+H]⁺ calcd for C₂₀H₃₀N₃OSi: 356.2153, found 356.2152.

### Step C. N-[4-[tert-butyl(dimethyl)silyl]oxyphenyl]-N-(5-cyano-1-ethyl-2-methyl-pyrrol-3-yl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

The title compound was obtained using the method described in Preparation VIIb and the product from Step B as the appropriate aniline for the amide formation.

¹H NMR (500 MHz, DMSO-d6) δ ppm 7.40-6.32 (m, 11H), 5.54-4.89 (s, 1H), 5.35-2.08 (m, 14H), 3.41-3 (s, 3H), 2.44-1.81 (s, 6H), 1.36-0.44 (m, 6H), 0.88 (s, 9H), 0.12 (s, 6H).

HPLC-MS-ESI: (m/z) [M+H]⁺ calcd for C₄₇H₅₇N₆O₃Si: 781.4, found 781.4.

### Step D: N-(5-cyano-1-ethyl-2-methyl-pyrrol-3-yl)-N-(4-hydroxyphenyl)-1,2-dimethyl-5-[7-[(3R)-3-methyl-3,4-dihydro-1H-isoquinoline-2-carbonyl-2-[2-[4-(2-morpholinoethoxy)phenyl]acetyl]-3,4-dihydro-1H-isoquinolin-6-yl]pyrrole-3-carboxamide

Using General procedure 4c, starting from Preparation Va as the appropriate acid (65 mg, 2.5 eq.) and the product of Step C (90 mg, 0.097 mmol) as the appropriate amine, the desired product was obtained (15.4 mg, 17%).

HRMS-ESI (m/z) [M+H]⁺ calcd for C₅₅H₆₀N₇O₆: 914.4600, found 914.4600.

### Example 42: N-(5-cyano-1,2-dimethyl-1H-pyrrol-3-yl)-5-{2-[2-(4-{2-[(2R or S,6R or S)-2,6-dimethylmorpholin-4-yl]ethoxy}phenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

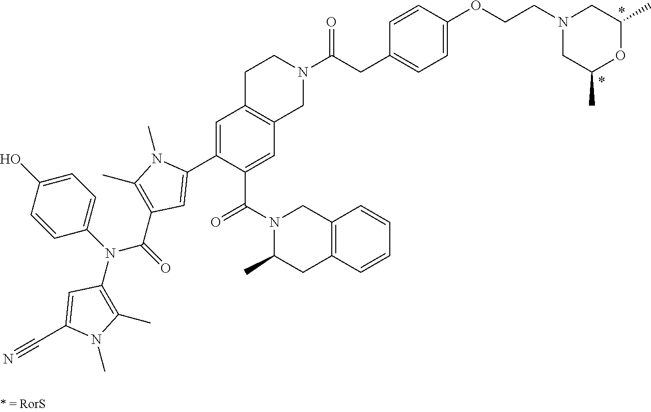 [SMILES: Cc1c(N(C(=O)c2cc(-c3cc4c(cc3C(=O)N3Cc5ccccc5C[C@H]3C)CN(C(=O)Cc3ccc(OCCN5C[C@H](C)O[C@@H](C)C5)cc3)CC4)n(C)c2C)c2ccc(O)cc2)cc(C#N)n1C]

### Step A: 2-[4-[2-[(2R or S,6R or S)-2,6-dimethylmorpholin-4-yl]ethoxy]phenyl]acetic acid

Using the General procedure 8, starting from the Diastereomer 2 Enantiomer 2 of Step A of Example 39 (210 mg, 0.68 mmol), the desired product was obtained (150 mg, 56%).

¹H NMR (500 MHz, DMSO-d6) δ ppm 11.90 (brs, 1H), 7.15 (d, 2H), 6.87 (d, 2H), 4.04 (t, 2H), 3.88 (m, 2H), 3.47 (s, 2H), 2.61 (m, 2H), 2.49/2.17 (dd+dd, 4H), 1.12 (d, 6H); ¹³C NMR (125 MHz, DMSO-d6) δ ppm 173.5, 157.7, 130.8, 127.5, 114.8, 66.2, 65.8, 59.2, 57.4, 40.3, 18.6.

HRMS-ESI (m/z) [M+H]⁺ calcd for C₁₆H₂₄NO₄: 294.1705, found 294.1722.

### Step B: N-(5-cyano-1,2-dimethyl-1H-pyrrol-3-yl)-5-{2-[2-(4-{2-[(2R or S,6R or S)-2,6-dimethylmorpholin-4-yl]ethoxy]phenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step A (111 mg, 3.6 eq.) as the appropriate carboxylic acid and Preparation VIIb (80 mg, 0.10 mmol) as the appropriate amine, afforded the title compound (60 mg, 62%).

HRMS-ESI (m/z) [M+H]⁺ calcd for C₅₆H₆₂N₇O₆: 928.4762, found 928.4757.

### Example 43: N-(5-cyano-1,2-dimethylpyrrol-3-yl)-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

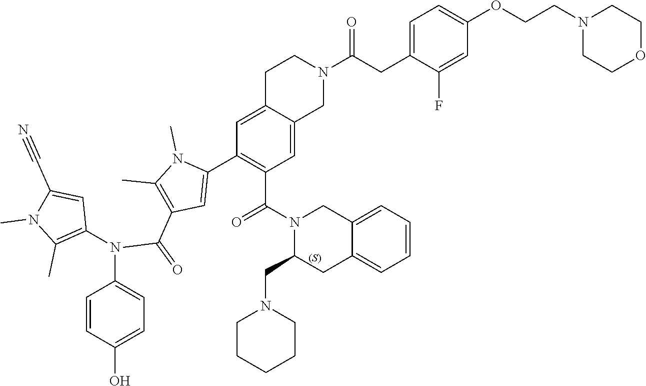

Using General procedure 4a and Preparation IVb (130 mg, 0.18 mmol, 1 eq.) as the appropriate amine and Preparation Vb (55 mg, 0.19 mmol, 1.1 eq.) as the appropriate carboxylic acid afforded the title product (45.4 mg, 0.05 mmol, 26%).

HRMS calcd for C₅₉H₆₅FN₈O₆: 1000.5011, found 1001.5094 [M+H]⁺

### Example 44: 5-{2-[2-(4-chloro-2-fluorophenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(5-cyano-1,2-dimethylpyrrol-3-yl)-N-(4-hydroxyphenyl)-1,2-dimethylpyrrole-3-carboxamide

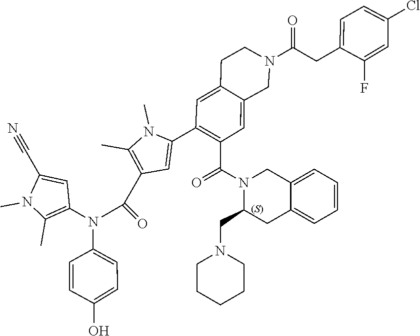

Using General procedure 4a and Preparation IVb (130 mg, 0.18 mmol, 1 eq.) as the appropriate amine and 4-chloro-2-fluorophenylacetic acid (37 mg, 0.19 mmol, 1.1 eq.) as the appropriate carboxylic acid afforded the title product (72.7 mg, 0.08 mmol, 45%).

HRMS calcd for C₅₃H₅₃ClFN₇O₄: 905.3832, found 906.3913 [M+H]⁺

### Example 45: 5-[2-(2-{4-[(9aS)-octahydropyrazino[2,1-c][1,4]oxazin-8-yl]-2-fluorophenyl}acetyl)-7-[(3R)-3-methyl-1,2,3,4-tetrahydroisoquinoline-2-carbonyl]-1,2,3,4-tetrahydroisoquinolin-6-yl]-N-(5-cyano-1,2-dimethyl-1H-pyrrol-3-yl)-N-(4-hydroxyphenyl)-1,2-dimethyl-1H-pyrrole-3-carboxamide

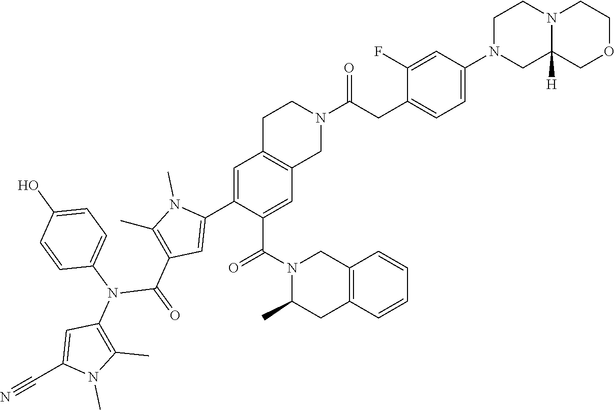 [SMILES: Cc1c(N(C(=O)c2cc(-c3cc4c(cc3C(=O)N3Cc5ccccc5C[C@H]3C)CN(C(=O)Cc3ccc(N5CCN6CCOC[C@@H]6C5)cc3F)CC4)n(C)c2C)c2ccc(O)cc2)cc(C#N)n1C]

### Step A: 2-[4-[(9aS)-3,4,6,7,9,9a-hexahydro-1H-pyrazino[2,1-c][1,4]oxazin-8-yl]-2-fluoro-phenyl]acetic acid hydrochloride

To a mixture of methyl 2-(4-bromo-2-fluoro-phenyl)acetate (240 mg, 0.8 eq.) and (9aS)-1,3,4,6,7,8,9,9a-octahydropyrazino[2,1-c][1,4]oxazine, hydrogen chloride (1:2) (225 mg, 1.26 mmol) in 1,4-dioxane (5.0 mL) were added Cs₂CO₃ (469 mg, 1.1 eq.) and [2-(2-aminoethyl)phenyl]-chloro-palladium, dicyclohexyl-[2-(2,4,6-triisopropylphenyl)phenyl]phosphane (1:1) (37 mg, 0.04 eq.). The reaction was stirred until complete conversion was observed. After filtration, the crude product was hydrolysed using the General procedure 8, purified, treated with 2 mL of 2 M HCl solution, and freeze dried to give the title compound (279 mg, 75%).

¹H NMR (500 MHz, DMSO-d6) δ ppm 12.19 (brs, 1H), 7.18 (t, 1H), 6.84 (dd, 1H), 6.76 (dd, 1H), 4.00/3.70 (dd+dd, 2H), 3.97 (dd, 2H), 3.90/3.30 (dt+dd, 2H), 3.85/2.94 (dt+dd, 2H), 3.50 (s, 2H), 3.48 (brm, 1H), 3.43/3.22 (dd+dd, 2H), 3.35/3.20 (dd+dd, 2H); ¹³C NMR (125 MHz, DMSO-d6) δ ppm 172.5, 161.8, 150.5, 132.6, 113.3, 111.5, 103.0, 65.5, 63.9, 60.2, 51.7, 51.4, 46.3, 45.4, 34.0.

HRMS-ESI (m/z) [M+H]⁺ calcd for C₁₅H₁₉FN₂O₃: 295.1452, found 295.1453.

### Step B: 5-[2-(2-{4-[(9aS)-octahydropyrazino[2,1-c][1,4]oxazin-8-yl]-2-fluorophenyl}acetyl)-7-[(3R)-3-methyl-1,2,3,4-tetrahydroisoquinoline-2-carbonyl]-1,2,3,4-tetrahydroisoquinolin-6-yl]-N-(5-cyano-1,2-dimethyl-1H-pyrrol-3-yl)-N-(4-hydroxyphenyl)-1,2-dimethyl-1H-pyrrole-3-carboxamide

Using General procedure 4c, starting from the product of Step A (150 mg, 4.3 eq.) as the appropriate carboxylic acid and Preparation VIIb (80 mg, 0.10 mmol) as the appropriate amine, afforded the title compound (51 mg, 53%).

HRMS-ESI (m/z) [M+H]⁺ calcd for C₅₅H₅₈FN₈O₅: 929.4509, found 929.4513.

### Example 46: 5-[2-(2-{2-chloro-4-[2-(morpholin-4-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(5-cyano-1,2-dimethyl-1H-pyrrol-3-yl)-N-(4-hydroxyphenyl)-1,2-dimethyl-1H-pyrrole-3-carboxamide

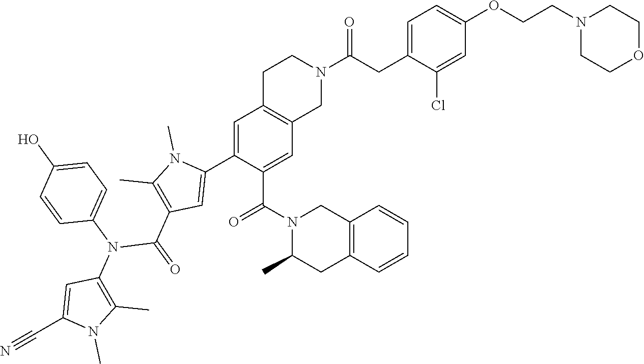 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3Cl)CC4)n(C)c2C)c2ccc(O)cc2)cc(C#N)n1C]

### Step A: 2-[2-chloro-4-(2-morpholinoethoxy)phenyl]acetic acid, TEA salt

Using General procedure 7b and then General procedure 8, starting from methyl 2-(2-chloro-4-hydroxy-phenyl)acetate (150 mg, 0.75 mmol) as the appropriate phenol and 2-morpholinoethanol (147 mg, 1.5 eq.) as the appropriate alcohol, followed with a purification step by automated flash chromatography using DCM and MeOH (1% TEA) as eluents afforded the TEA salt of the title product (236 mg, 79%).

¹H NMR (500 MHz, DMSO-d6) δ ppm 12.24 (brs, 1H), 7.27 (d, 1H), 7.04 (d, 1H), 6.88 (dd, 1H), 4.09 (t, 2H), 3.62 (s, 2H), 3.57 (m, 4H), 2.67 (t, 2H), 2.6-2.3 (brs, 4H); ¹³C NMR (125 MHz, DMSO-d6) δ ppm 133.1, 115.3, 114.2, 66.6, 66.2, 57.3, 54, 38.2.

HRMS-ESI (m/z) [M+H]⁺ calcd for C₁₄H₁₉ClNO₄:300.0997, found 300.0998.

### Step B: 5-[2-(2-{2-chloro-4-[2-(morpholin-4-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl-N-(5-cyano-1,2-dimethyl-1H-pyrrol-3-yl)-N-(4-hydroxyphenyl)-1,2-dimethyl-1H-pyrrole-3-carboxamide

Using General procedure 4c, starting from the product of Step A (122 mg, 2.6 eq.) as the appropriate carboxylic acid and Preparation VIIb (90 mg, 0.12 mmol) as the appropriate amine, afforded the title compound (68.8 mg, 63%).

HRMS-ESI (m/z) [M+H]⁺ calcd for C₅₄H₅₇C₁N₇O₆: 934.4053, found 934.4058.

### Example 47: N-(5-cyano-1,2-dimethyl-1H-pyrrol-3-yl)-5-{2-[2-(4-{2-[4-(2,2-difluoroethyl)piperazin-1-yl]ethoxy}phenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

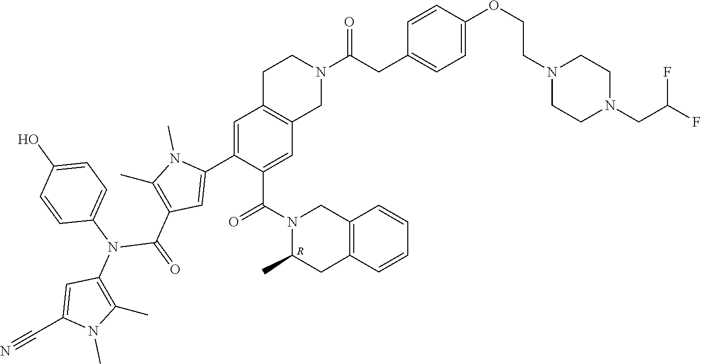

### Step A: 2-[4-[2-[4-(2,2-difluoroethyl)piperazin-1-yl]ethoxy]phenyl]acetic acid

Using General procedure 7d and then General procedure 8, starting from methyl 2-[4-(2-bromoethoxy)phenyl]acetate (160 mg, 0.58 mmol) as the appropriate alkyl halide and 1-(2,2-difluoroethyl)piperazine (245 mg, 1.2 eq) as the appropriate amine, the title product was obtained (145 mg, 76%).

¹H NMR (500 MHz, DMSO-d6) δ ppm 12.2 (brs, 1H), 7.16 (d, J=8.5 Hz, 2H), 8.87 (d, 8.5 Hz, 2H), 6.27 (t, J=4.2 Hz, 0.25H), 6.13 (t, J=4.2 Hz, 0.5H), 5.99 (t, J=4.2 Hz, 0.25H), 4.05 (t, J=5.6 Hz, 2H), 3.34 (s, 8H), 2.70 (m+m, 4H), 2.53 (m, 2H).

HPLC-MS-ESI (m/z) [M+H]⁺ calcd for C₁₆H₂₃F₂N₂O₃: 329.1671, found 329.2.

### Step B: N-(5-cyano-1,2-dimethyl-1H-pyrrol-3-yl)-5-{2-[2-(4-{2-[4-(2,2-difluoroethyl)piperazin-1-yl]ethoxy]phenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step A (60 mg, 1.5 eq.) as the appropriate carboxylic acid and Preparation VIIb (100 mg, 0.10 mmol) as the appropriate amine, afforded the title product (38.8 mg, 30%).

HRMS-ESI (m/z) [M+H]⁺ calcd for C₅₆H₆₁F₂N₈O₅: 963.4727, found 963.4729.

### Example 48: N-(5-cyano-1,2-dimethylpyrrol-3-yl)-5-[2-(2-{4-[1-(2,2-difluoroethyl)piperidin-4-yl]-2-fluorophenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

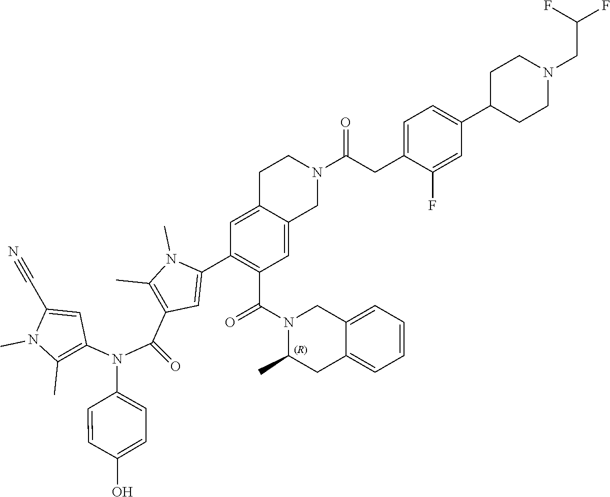

### Step A: tert-butyl 4-[3-fluoro-4-(2-methoxy-2-oxoethyl)phenyl]-3,6-dihydro-2H-pyridine-1-carboxylate

Methyl 2-(4-bromo-2-fluorophenyl)acetate (0.33 mL, 2.02 mmol, 1 eq.), 3,6-dihydro-2H-pyridine-1-N-Boc-4-boronic acid, pinacol ester (688 mg, 2.23 mmol, 1.1 eq.) and K₂CO₃ (839 mg, 6.07 mmol, 3 eq.) were combined in THE (10 mL) and water (1 mL) and the mixture was sparged with N₂. Pd(dppf)Cl₂ DCM (83 mg, 0.1 mmol, 0.05 eq.) was added and the mixture was heated at 110° C. under microwave irradiation for 1 h. The mixture was allowed to cool to rt and partitioned between DCM and brine. The phases were separated, and the organic phase was dried (PTFE phase separator) and concentrated in vacuo. Purification by automated flash chromatography eluting with a gradient of 0-31% EtOAc in heptane afforded the title product (707 mg, 2.03 mmol, quant.).

LRMS calcd for C₁₉H₂₄FNO₄: 349.2, found 250.1 [M-Boc+H]⁺

¹H NMR (400 MHz, DMSO-d6) δ ppm: 7.36-7.20 (m, 3H), 6.28-6.18 (m, 1H), 4.06-3.94 (m, 2H), 3.73-3.70 (m, 2H), 3.62 (s, 3H), 3.53 (t, J=5.7 Hz, 2H), 2.48-2.42 (m, 2H), 1.42 (s, 9H).

### Step B: tert-butyl 4-[3-fluoro-4-(2-methoxy-2-oxoethyl)phenyl]piperidine-1-carboxylate

To a solution of the product from Step A (707 mg, 2.02 mmol, 1 eq.) in MeOH (30 mL) was added 10% Pd/C (25 mg). The mixture was evacuated and backfilled with N₂, then evacuated and flushed with H₂ and then shaken at rt for 4 h under an atmosphere of H₂. The mixture was filtered through a celite cartridge, washing with MeOH and the solvent removed in vacuo to afford the title product (679 mg, 1.93 mmol, 95%).

¹H NMR (400 MHz, DMSO-d6) δ ppm: 7.25 (t, J=7.9 Hz, 1H), 7.11-7.02 (m, 2H), 4.13-3.99 (m, 2H), 3.70-3.65 (m, 2H), 3.61 (s, 3H), 2.92-2.63 (m, 3H), 1.79-1.70 (m, 2H), 1.54-1.37 (m, 11H).

### Step C: methyl 2-[2-fluoro-4-(piperidin-4-yl)phenyl]acetate

TFA (3 mL) was added to a solution of the product from Step B (679 mg, 1.93 mmol, 1 eq.) in DCM (10 mL) and the reaction mixture was stirred at rt for 2 h. The solvents were removed in vacuo, and the residue was partitioned between DCM and sat. aq. NaHCO₃ solution. The organic phase was collected (PTFE phase separator) and concentrated in vacuo to afford the title product (488 mg, 1.94 mmol, 100%).

LRMS calcd for C₁₄H₁₈FNO₂: 251.1, found 252 [M+H]⁺

¹H NMR (400 MHz, DMSO-d6) δ ppm: 7.25 (t, J=8.1 Hz, 1H), 7.07-6.99 (m, 2H), 3.69-3.66 (m, 2H), 3.62 (s, 3H), 3.07-2.99 (m, 2H), 2.65-2.54 (m, 3H), 1.73-1.65 (m, 2H), 1.56-1.43 (m, 2H).

### Step D: methyl 2-{4-[1-(2,2-difluoroethyl)piperidin-4-yl]-2-fluorophenyl}acetate

2-Bromo-1,1-difluoroethane (90 μL, 1.05 mmol, 1.1 eq.) was added to a stirred suspension of the product from Step C (240 mg, 0.96 mmol, 1 eq.) and K₂CO₃ (528 mg, 3.82 mmol, 4 eq.) in DMF (2 mL) under a N₂ atmosphere and the mixture was heated at 100° C. under microwave irradiation for 1 h. The mixture was allowed to cool to rt and partitioned between DCM and sat. aq. NaHCO₃ solution. The organic phase was separated (PTFE phase separator) and concentrated in vacuo. Purification by automated flash chromatography eluting with a gradient of 0-80% EtOAc in heptane afforded the title product (164 mg, 0.52 mmol, 54%).

LRMS calcd for C₁₆H₂₀F₃NO₂: 315.1, found 316.2 [M+H]⁺

¹H NMR (400 MHz, DMSO-d6) δ ppm: 7.24 (t, J=8.1 Hz, 1H), 7.10-7.01 (m, 2H), 6.31-5.98 (m, 1H), 3.70-3.65 (m, 2H), 3.61 (s, 3H), 3.03-2.95 (m, 2H), 2.74 (td, J=15.7, 4.4 Hz, 2H), 2.56-2.44 (m, 1H), 2.29-2.20 (m, 2H), 1.77-1.69 (m, 2H), 1.69-1.56 (m, 2H).

### Step E: {4-[1-(2,2-difluoroethyl)piperidin-4-yl]-2-fluorophenyl}acetic acid

To a solution of the product from Step D (164 mg, 0.52 mmol, 1 eq.) in MeOH (4 mL) was added LiOH·H₂O (44 mg, 1.04 mmol, 2 eq.) and the mixture was heated at 100° C. under microwave irradiation for 30 mins. The mixture was allowed to cool to rt, neutralised with 2 M aq. HCl solution and concentrated in vacuo. Purification by reverse phase automated flash chromatography eluting with a gradient of 10-100% MeCN in water afforded the title product (139 mg, 0.46 mmol, 89%).

LRMS calcd for C₁₅H₁₈F₃NO₂: 301.1, found 302.2 [M+H]⁺

¹H NMR (400 MHz, DMSO-d6) δ ppm: 12.42 (s, 1H), 7.23 (t, J=8.1 Hz, 1H), 7.09-6.99 (m, 2H), 6.15 (tt, J=55.8, 4.3 Hz, 1H), 3.58-3.54 (m, 2H), 3.03-2.95 (m, 2H), 2.74 (td, J=15.7, 4.4 Hz, 2H), 2.55-2.45 (m, 1H), 2.29-2.20 (m, 2H), 1.77-1.57 (m, 4H).

### Step F: N-(5-cyano-1,2-dimethylpyrrol-3-yl)-5-[2-(2-{4-[1-(2,2-difluoroethyl)piperidin-4-yl]-2-fluorophenyl}acetyl)-7-{[(3R)-3-methyl-3,4-dihydro-1H-isoquinolin-2-yl]carbonyl}-3,4-dihydro-1H-isoquinolin-6-yl-N-(4-hydroxyphenyl)-1,2-dimethylpyrrole-3-carboxamide

Using General procedure 4a and Preparation IVa (130 mg, 0.2 mmol, 1 eq.) as the appropriate amine and the product from Step E (66 mg, 0.22 mmol, 1.1 eq.) as the appropriate carboxylic acid afforded the title product (97.2 mg, 0.1 mmol, 52%).

HRMS calcd for C₅₅H₅₆F₃N₇O₄: 935.4346, found 936.4436 [M+H]⁺

### Example 49: N-(5-cyano-1,2-dimethyl-1H-pyrrol-3-yl)-5-[2-(2-{4-[2-(3-fluoroazetidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

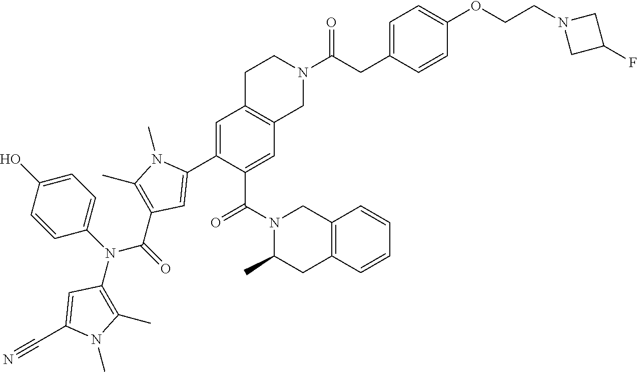 [SMILES: Cc1c(N(C(=O)c2cc(-c3cc4c(cc3C(=O)N3Cc5ccccc5C[C@H]3C)CN(C(=O)Cc3ccc(OCCN5CC(F)C5)cc3)CC4)n(C)c2C)c2ccc(O)cc2)cc(C#N)n1C]

### Step A: 2-[4-[2-(3-fluoroazetidin-1-yl)ethoxy]phenyl]acetic acid

Using General procedure 7d and then General procedure 8, starting from methyl 2-[4-(2-bromoethoxy)phenyl]acetate (150 mg, 0.55 mmol) as the appropriate alkyl halide and 3-fluoroazetidine hydrochloride (122.5 mg, 2 eq) as the appropriate amine, the title product was obtained (108 mg, 77.6%).

¹H NMR (500 MHz, DMSO-d6) δ ppm 12.88 (brs, 1H), 7.13 (m, 2H), 6.81 (m, 2H), 5.14 (dm, 1H), 3.91 (t, 2H), 3.61/3.17 (m+m, 2H), 3.39 (s, 2H), 2.79 (t, 2H); ¹³C NMR (125 MHz, DMSO-d6) δ ppm 173.8, 130.8, 114.5, 83.4, 66.8, 62.1, 57.9, 41.4.

HRMS-ESI (m/z) [M+H]⁺ calcd for C₁₃H₁₇FNO₃: 254.1186, found 254.1187.

### Step B: N-(5-cyano-1,2-dimethyl-1H-pyrrol-3-yl)-5-[2-(2-{4-[2-(3-fluoroazetidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from the product of Step A (64 mg, 2.5 eq.) as the appropriate carboxylic acid and Preparation VIIb (70 mg, 0.091 mmol) as the appropriate amine, afforded the title product (40.1 mg, 50%).

HRMS-ESI (m/z) [M+H]⁺ calcd for C₅₃H₅₅FN₇O: 888.4243, found 888.4247.

### Example 50: N-(5-cyano-1,2-dimethyl-1H-pyrrol-3-yl)-5-[2-(2-{4-[2-(3,3-difluoropyrrolidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

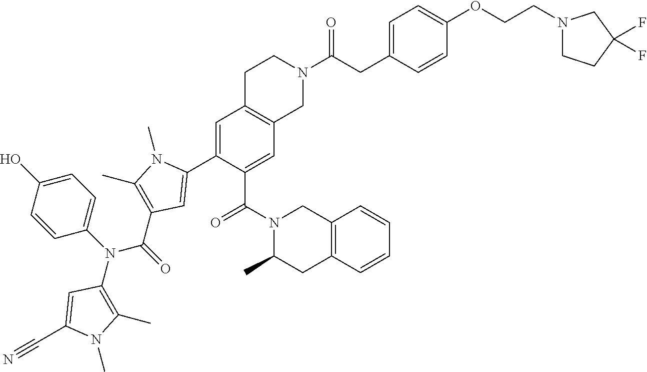 [SMILES: Cc1c(N(C(=O)c2cc(-c3cc4c(cc3C(=O)N3Cc5ccccc5C[C@H]3C)CN(C(=O)Cc3ccc(OCCN5CCC(F)(F)C5)cc3)CC4)n(C)c2C)c2ccc(O)cc2)cc(C#N)n1C]

### Step A: 2-[4-[2-(3,3-difluoropyrrolidin-1-yl)ethoxy]phenyl]acetic acid

Using General procedure 7d and then General procedure 8, starting from methyl 2-[4-(2-bromoethoxy)phenyl]acetate (150 mg, 0.55 mmol) and 3,3-difluoropyrrolidine hydrochloride (157.7 mg, 2 eq.) as reactants, the title product was obtained (48 mg, 31%).

¹H NMR (500 MHz, DMSO-d6) δ ppm 12.26 (brs, 1H), 7.15 (m, 2H), 6.87 (m, 2H), 4.04 (t, 2H), 3.47 (s, 2H), 2.97 (t, 2H), 2.81 (t, 2H), 2.78 (t, 2H), 2.23 (m, 2H); ¹³C NMR (125 MHz, DMSO-d6) δ ppm 173.5, 131.1, 130.9, 114.7, 66.6, 62.1, 54, 52.6, 40.3, 35.7.

HRMS-ESI (m/z) [M+H]⁺ calcd for C₁₄H₁₈F₂NO₃: 286.1249, found 286.1253.

### Step B: N-(5-cyano-1,2-dimethyl-1H-pyrrol-3-yl)-5-[2-(2-{4-[2-(3,3-difluoropyrrolidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from the product of Step A (52 mg, 2.5 eq.) as the appropriate carboxylic acid and Preparation VIIb (50 mg, 0.065 mmol.) as the appropriate amine, afforded the title product (25.1 mg, 42%).

HRMS-ESI (m/z) [M+H]⁺ calcd for C₅₄H₅₆F₂N₇O₅: 920.4306, found 920.4304.

### Example 51: N-(5-cyano-1,2-dimethylpyrrol-3-yl)-5-[2-(2-{4-[2-(4,4-difluoropiperidin-1-yl)ethyl]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

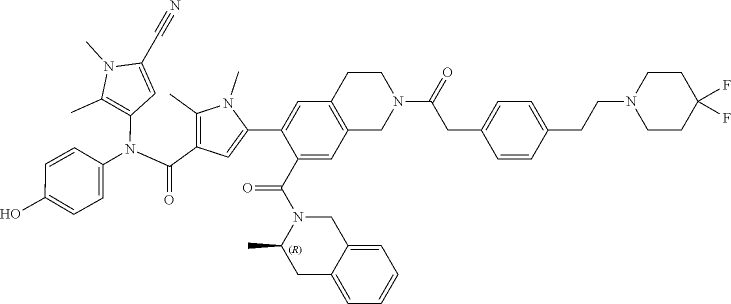

### Step A: {4-[(E)-2-ethoxyethenyl]phenyl}acetic acid

To a solution of the product from Example 38, Step A (360 mg, 1.63 mmol, 1 eq.) in MeOH (5 mL) was added 2 M aq. NaOH solution (0.82 mL, 1.63 mmol, 1 eq.) and the mixture was stirred at rt for 18 h. The mixture was concentrated in vacuo, acidified with 2 M aq. HCl solution and extracted with DCM. The combined organic extracts were dried over MgSO₄ and concentrated in vacuo to afford the title product (311 mg, 1.51 mmol, 92%).

¹H NMR (400 MHz, DMSO-d6) δ ppm: 7.24-7.14 (m, 3H), 7.14-7.08 (m, 2H), 5.80 (d, J=13.0 Hz, 1H), 3.87 (q, J=7.0 Hz, 2H), 3.48 (s, 2H), 1.24 (t, J=7.0 Hz, 3H).

### Step B: {4-[2-(4,4-difluoropiperidin-1-yl)ethyl]phenyl}acetic acid

To a solution of the product from Step A (222 mg, 1.08 mmol, 1 eq.) in acetone (5 mL) was added 2 M aq. HCl solution (0.54 mL, 1.08 mmol, 1 eq.) and the mixture stirred at rt for 18 h. The mixture was diluted with DCM and dried over MgSO₄ and concentrated in vacuo. To a solution of the intermediate in DCM (5 mL) was added 4,4-difluoropiperidine (249 mg, 1.58 mmol, 1.2 eq.) and TEA (220 μL, 1.58 mmol, 1.2 eq.) followed by NaBH(OAc)₃ (559 mg, 2.64 mmol, 2 eq.) and the mixture was stirred at rt for 18 h. The mixture was quenched with MeOH, concentrated in vacuo and purified by reverse phase automated flash chromatography eluting with a gradient of 10-100% MeCN in water to afford the title product (183 mg, 0.65 mmol, 49%).

¹H NMR (400 MHz, DMSO-d6) δ ppm: 7.15-7.08 (m, 4H), 3.36 (s, 2H), 2.72-2.65 (m, 2H), 2.59-2.52 (m, 6H), 2.00-1.87 (m, 4H).

### Step C: N-(5-cyano-1,2-dimethylpyrrol-3-yl)-5-[2-(2-{4-[2-(4,4-difluoropiperidin-1-yl)ethyl]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a, and Preparation IVa (110 mg, 0.17 mmol, 1 eq) as the appropriate amine and the product from Step B (95 mg, 0.34 mmol, 2 eq.) as the appropriate carboxylic acid afforded the title product (23 mg, 0.02 mmol, 15%).

HRMS calcd for C₅₅H₅₇F₂N₇O₄: 917.4440, found 918.4522 [M+H]⁺

### Example 52: N-(5-cyano-1,2-dimethylpyrrol-3-yl)-5-[2-(2-{2-fluoro-4-[2-(oxan-4-yl)ethoxy]phenyl}acetyl)-7-{[(3S)-3-(methoxymethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

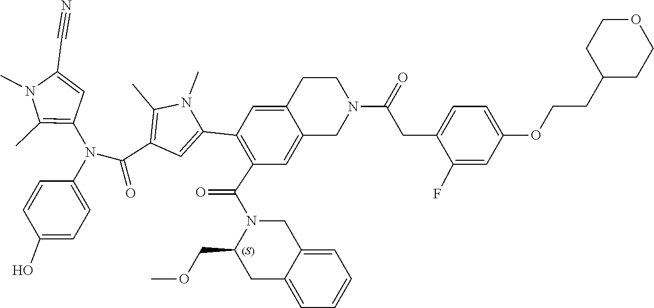

### Step A: tert-butyl (3S)-3-(hydroxymethyl)-3,4-dihydro-1H-isoquinoline-2-carboxylate

To a solution of (S)-1,2,3,4-tetrahydroisoquinoline-3-methanol (2 g, 12.25 mmol, 1 eq.) in DCM (50 mL) was added TEA (6.14 mL, 44.11 mmol, 3.6 eq.) and di-tert-butyl dicarbonate, (3.15 mL, 14.7 mmol, 1.2 eq.) and the mixture was stirred at rt under N₂ for 18 h. The mixture was diluted with sat. aq. NaHCO₃ solution and the organic layer was separated, washed with brine, dried over MgSO₄ and concentrated in vacuo. Purification by automated flash chromatography eluting with a gradient of 0-100% EtOAc in heptane afforded the title product (2.96 g, 11.23 mmol, 92%).

LRMS calcd for C₁₅H₂₁NO₃: 263.2, found 164.0 [M-Boc+H]⁺

¹H NMR (400 MHz, DMSO-d6) δ ppm: 7.21-7.12 (m, 4H), 4.77 (t, J=5.4 Hz, 1H), 4.61 (d, J=16.7 Hz, 1H), 4.37-4.08 (br m, 2H), 3.38-3.24 (br m, 1H), 3.18-3.06 (br m, 1H), 2.93-2.79 (m, 2H), 1.43 (s, 9H).

### Step B: tert-butyl (3S)-3-(methoxymethyl)-3,4-dihydro-1H-isoquinoline-2-carboxylate

To a solution of the product from Step A (440 mg, 1.67 mmol, 1 eq.) in DMF (10 mL), cooled to 0° C., was added NaH (60% dispersion in mineral oil; 100 mg, 2.51 mmol, 1.5 eq.) and the mixture was stirred for 10 mins. Iodomethane (156 μL, 2.51 mmol, 1.5 eq.) was added and the reaction was allowed to warm to rt. Then, it was stirred for 18 h. The mixture was partitioned between EtOAc and sat. aq. NaHCO₃, the layers were separated, and the organic phase was washed with brine, dried over MgSO₄ and concentrated in vacuo to afford the title product (450 mg, 1.62 mmol, 97%).

LRMS calcd for C₁₆H₂₃NO₃: 277.2, found 178.0 [M-Boc+H]⁺

¹H NMR (400 MHz, DMSO-d6) δ ppm: 7.20-7.13 (m, 4H), 4.62 (d, J=16.8 Hz, 1H), 4.56-4.33 (br m, 1H), 4.28-4.08 (br m, 1H), 3.29-3.17 (m, 4H), 3.13-3.04 (m, 1H), 3.00-2.89 (m, 1H), 2.80-2.71 (m, 1H), 1.43 (s, 9H).

### Step C: (3S)-3-(methoxymethyl)-1,2,3,4-tetrahydroisoquinoline hydrochloride

To a solution of the product from Step B (450 mg, 1.62 mmol, 1 eq.) in THE (10 mL) was added 4 M HCl in dioxane (10 mL) and the mixture was stirred at rt for 18 h. The mixture was concentrated in vacuo and then triturated with Et₂O and heptane to afford the title product (257 mg, 1.45 mmol, 89%).

LRMS calcd for C₁₁H₁₅NO: 177.1, found 178.0 [M+H]⁺

¹H NMR (400 MHz, DMSO-d6) δ ppm: 9.85-9.37 (br m, 2H), 7.30-7.18 (m, 4H), 4.36-4.17 (m, 2H), 3.75-3.59 (m, 3H), 3.38 (s, 3H), 3.03-2.89 (m, 2H).

### Step D: benzyl 6-[4-(ethoxycarbonyl)-1,5-dimethylpyrrol-2-yl]-7-{[(3S)-3-(methoxymethyl)-3,4-dihydro-1H-isoquinolin-2-yl]carbonyl}-3,4-dihydro-1H-isoquinoline-2-carboxylate

Using General procedure 4b and Preparation I (450 mg, 0.94 mmol, 1 eq) as the appropriate carboxylic acid and the product from Step C (222 mg, 1.04 mmol, 1.1 eq.) as the appropriate amine afforded the title product which was used directly in the subsequent step without further purification.

LRMS calcd for C₃₈H₄₁N₃O₆: 635.3, found 636.4 [M+H]⁺

### Step E: ethyl 5-(7-{[(3S)-3-(methoxymethyl)-3,4-dihydro-1H-isoquinolin-2-yl]carbonyl}-1,2,3,4-tetrahydroisoquinolin-6-yl)-1,2-dimethylpyrrole-3-carboxylate

To a solution of the product from Step D (600 mg, 0.94 mmol, 1 eq.) in MeOH (25 mL) was added 10% Pd/C (25 mg). The mixture was evacuated and backfilled with N₂, then evacuated and flushed with H₂ and then shaken at rt for 18 h under an atmosphere of H₂. The mixture was filtered through a celite cartridge, washing with MeOH. The solvent was removed in vacuo and the residue was loaded onto a pre-washed (MeOH) 10 g SCX-2 cartridge, washing with DCM and MeOH and then the product was eluted with 4:1 DCM/7 M NH₃ in MeOH. The solvents were removed in vacuo to afford the title product which was used directly in the subsequent step without further purification.

LRMS calcd for C₃₀H₃₅N₃O₄: 501.3, found 502.4 [M+H]⁺

### Step F: tert-butyl 6-[4-(ethoxycarbonyl)-1,5-dimethylpyrrol-2-yl]-7-{[(3S)-3-(methoxymethyl)-3,4-dihydro-1H-isoquinolin-2-yl]carbonyl}-3,4-dihydro-1H-isoquinoline-2-carboxylate

To a solution of the product from Step E (480 mg, 0.96 mmol, 1 eq.) in THE (10 mL) and water (2 mL) was added di-tert-butyl dicarbonate (0.21 mL, 1 mmol, 1.05 eq.) and TEA (0.27 mL, 1.91 mmol, 2 eq.) and the mixture was stirred at rt for 24 h. The solvent was evaporated under reduced pressure and the residue partitioned between EtOAc and sat. aq. NH₄Cl solution. Water was added, the phases were separated, and the organic phase was washed with brine. The organic phase was dried over MgSO₄ and concentrated in vacuo to afford the title product (476 mg, 0.79 mmol, 83%).

LRMS calcd for C₃₅H₄₃N₃O₆: 601.3, found 602.4 [M+H]⁺

### Step G: 5-[2-(tert-butoxycarbonyl)-7-{[(3S)-3-(methoxymethyl)-3,4-dihydro-1H-isoquinolin-2-yl]carbonyl}-3,4-dihydro-1H-isoquinolin-6-yl]-1,2-dimethylpyrrole-3-carboxylic acid

To a solution of the product from Step F (476 mg, 0.79 mmol, 1 eq.) in MeOH (10 mL) and water (2 mL) was added LiOH·H₂O (133 mg, 3.16 mmol, 4 eq.) and the mixture was refluxed at 100° C. for 48 h. The mixture was allowed to cool to rt and concentrated in vacuo. The aqueous residue was neutralised with 1 M aq. HCl solution and extracted with DCM. The combined organic extracts were dried over MgSO₄ and concentrated in vacuo. Purification by automated flash chromatography eluting with a gradient of 20-100% EtOAc in heptane afforded the title product (318 mg, 0.55 mmol, 70%).

LRMS calcd for C₃₃H₃₉N₃O₆: 573.3, found 574.4 [M+H]⁺

### Step H: 1,5-dimethyl-4-({4-[(triisopropylsilyl)oxy]phenyl}amino)pyrrole-2-carbonitrile

Using General procedure 1a, and the product from Preparation IVa, Step A (7.99 g, 40.14 mmol, 1 eq) and Preparation IIIb (12.79 g, 48.17 mmol, 1.2 eq) as the appropriate aniline afforded the title product (10.68 g, 27.84 mmol, 69%).

LRMS calcd for C₂₂H₃₃N₃OSi: 383.2, found: 384.4 [M+H]⁺

¹H NMR (400 MHz, DMSO-d6) δ ppm: 6.86 (s, 1H), 6.72 (s, 1H), 6.67-6.58 (m, 2H), 6.55-6.46 (m, 2H), 3.61 (s, 3H), 2.08 (s, 3H), 1.26-1.10 (m, 3H), 1.04 (d, J=7.2 Hz, 18H).

### Step I.: tert-butyl 6-{4-[(5-cyano-1,2-dimethylpyrrol-3-yl)([4-[(triisopropylsilyl)oxy]phenyl})carbamoyl]-1,5-dimethylpyrrol-2-yl}-7-{[(3S)-3-(methoxymethyl)-3,4-dihydro-1H-isoquinolin-2-yl]carbonyl}-3,4-dihydro-1H-isoquinoline-2-carboxylate

Using General procedure 2a, and the product from Step G (300 mg, 0.52 mmol, 1 eq.) as the appropriate acid and the product from Step H (321 mg, 0.84 mmol, 1.6 eq.) as the appropriate aniline afforded the title product (318 mg, 0.34 mmol, 65%.

LRMS calcd for C₅₅H₇₀N₆O₆Si: 938.5, found 939.6 [M+H]⁺

### Step J. N-(5-cyano-1,2-dimethylpyrrol-3-yl)-5-(7-{[(3S)-3-(methoxymethyl)-3,4-dihydro-1H-isoquinolin-2-yl]carbonyl}-1,2,3,4-tetrahydroisoquinolin-6-yl)-1,2-dimethyl-N-{4-[(triisopropylsilyl)oxy]phenyl}pyrrole-3-carboxamide

Using General procedure 3b, and the product from Step I (318 mg, 0.34 mmol, 1 eq.) as the appropriate silyl-derivative afforded the title product (176 mg, 0.21 mmol, 62%).

LRMS calcd for C₅₀H₆₂N₆O₄Si: 838.5, found 839.6 [M+H]⁺

### Step K: N-(5-cyano-1,2-dimethylpyrrol-3-yl)-5-[2-(2-{2-fluoro-4-[2-(oxan-4-yl)ethoxy]phenyl}acetyl)-7-{[(3S)-3-(methoxymethyl)-3,4-dihydro-1H-isoquinolin-2-yl]carbonyl}-3,4-dihydro-1H-isoquinolin-6-yl]-1,2-dimethyl-N-{4-[(triisopropylsilyl)oxy]phenyl}pyrrole-3-carboxamide

Using General procedure 4a, and the product from Step J (88 mg, 0.1 mmol, 1 eq) as the appropriate amine and the product from Example 34, Step B (36 mg, 0.13 mmol, 1.2 eq.) as the appropriate carboxylic acid afforded the title product (106 mg, 0.1 mmol, 92%).

LRMS calcd for C₆₅H₇₉FN₆O₇Si) 1102.6, found 1103.4 [M+H]⁺

### Step L: N-(5-cyano-1,2-dimethylpyrrol-3-yl)-5-[2-(2-{2-fluoro-4-[2-(oxan-4-yl)ethoxy]phenyl}acetyl)-7-{[(3S)-3-(methoxymethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 3c and the product from Step K (106 mg, 0.1 mmol, 1 eq.) as the appropriate silyl-derivative afforded the title product (41.7 mg, 0.04 mmol, 46%).

HRMS calcd for C₅₆H₅₉FN₆O₇: 946.4429, found 947.4509 [M+H]⁺

### Example 53: N-(5-cyano-1,2-dimethyl-1H-pyrrol-3-yl)-5-[2-(2-{4-[2-(4-fluoropiperidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

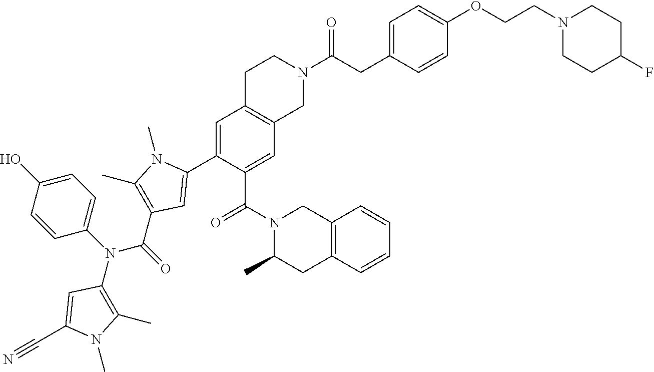 [SMILES: Cc1c(N(C(=O)c2cc(-c3cc4c(cc3C(=O)N3Cc5ccccc5C[C@H]3C)CN(C(=O)Cc3ccc(OCCN5CCC(F)CC5)cc3)CC4)n(C)c2C)c2ccc(O)cc2)cc(C#N)n1C]

### Step A: 2-[4-[2-(4-fluoro-1-piperidyl)ethoxy]phenyl]acetic acid

Using the General procedure 7d and then General procedure 8, starting from methyl 2-[4-(2-bromoethoxy)phenyl]acetate (160 mg, 0.59 mmol) as the appropriate alkyl halide and 4-fluoropiperidine hydrochloride (245 mg, 3.0 eq.) as the appropriate amine, followed with purification step by automated flash chromatography using DCM and MeOH (1% TEA) as eluents afforded the title product (157 mg, 95%).

¹H NMR (500 MHz, DMSO-d6) δ ppm 12.07 (br., 1H), 7.15 (dm, 2H), 6.87 (dm, 2H), 4.67 (m, 1H), 4.04 (t, 2H), 3.47 (s, 2H), 2.70 (t, 2H), 2.63/2.41 (br+br., 4H), 1.85/1.70 (m+m, 4H); ¹³C NMR (125 MHz, DMSO-d6) δ ppm 173.5, 157.7, 130.8, 127.5, 114.7, 88.9, 66, 56.8, 50.1, 40.2, 31.6.

HRMS-ESI (m/z) [M+H]⁺ calcd for C₁₅H₂₁FNO₃: 282.1499, found 282.1500.

### Step B: N-(5-cyano-1,2-dimethyl-1H-pyrrol-3-yl)-5-[2-(2-{4-[2-(4-fluoropiperidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from the product of Step A (73 mg, 2.5 eq.) as the appropriate carboxylic acid and Preparation VIIb (80 mg, 0.10 mmol) as the appropriate amine, afforded the title product (69 mg, 72%).

HRMS-ESI (m/z) [M+H]⁺ calcd for C₅₅H₅₉FN₇O₅: 916.4556, found 916.4561.

### Example 54: N-(5-cyano-1,2-dimethyl-pyrrol-3-yl)-5-[2-[2-[2-fluoro-4-(2-tetrahydropyran-4-ylethoxy)phenyl]acetyl]-7-[(3S)-3-(morpholinomethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

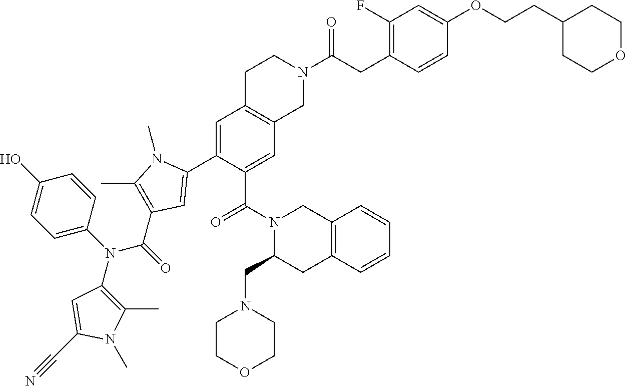 [SMILES: Cc1c(N(C(=O)c2cc(-c3cc4c(cc3C(=O)N3Cc5ccccc5C[C@H]3CN3CCOCC3)CN(C(=O)Cc3ccc(OCCC5CCOCC5)cc3F)CC4)n(C)c2C)c2ccc(O)cc2)cc(C#N)n1C]

### Step A: 1,2-dimethyl-5-[7-[(3S)-3-(morpholinomethyl)-3,4-dihydro-1H-isoquinoline-2-carbonyl]-1,2,3,4-tetrahydroisoquinolin-6-yl]pyrrole-3-carboxylic acid hydrochloride

Using the procedure described at Step A of Preparation VIIa, starting from Preparation I (3.0 g, 6.3 mmol) as the appropriate carboxylic acid and 4-[[(3S)-1,2,3,4-tetrahydroisoquinolin-3-yl]methyl]morpholine dihydrochloride (2.3 g, 1.2 eq., the synthesis is disclosed in WO2015/011164 Å1 as Preparation 2d) as the appropriate amine, the title compound was obtained (2.6 g, 69%).

¹H NMR (500 MHz, DMSO-d6) δ ppm 11.54/11.44 (br/br., 1H), 9.67/9.55 (br+br., 2H), 7.66 (s, 1H), 7.33/7.22 (br/s, 1H), 7.13-6.85 (m, 4H), 6.32/6.11 (br/s, 1H), 5.38 (m, 1H), 4.65/4.05 (d+d, 2H), 4.39/4.25 (dm+dm, 2H), 4.11-3.78 (m, 4H), 3.76-2.94 (m, 4H), 3.46/3.09 (s/s, 3H), 3.40 (m, 2H), 3.30/3.13 (m+m, 2H), 3.15-2.99 (m, 2H), 2.77/2.41 (dd+d, 2H), 1.92 (s, 3H); ¹³C NMR (125 MHz, DMSO-d6) δ ppm 131.5/129.9, 127.6, 111.1, 63.1, 55.3, 52.6/50.4, 44.4, 43.8, 41.7, 40.8, 32.2/31.8, 29.8, 25.1, 11.1.

HRMS-ESI (m/z) [M+H]⁺ calcd for C₃₁H₃₇N₄O₄: 529.2809, found 529.2814.

### Step B: 5-[2-(9H-fluoren-9-ylmethoxycarbonyl)-7-[(3S)-3-(morpholinomethyl)-3,4-dihydro-1H-isoquinoline-2-carbonyl]-3,4-dihydro-1H-isoquinolin-6-yl-1,2-dimethyl-pyrrole-3-carboxylic acid

To the product of Step A (1.36 g, 2.3 mmol.), NaHCO₃ (437 mg, 2.3 eq.) in 1,4-dioxane (13 mL) and water (13 mL) was added 9H-fluoren-9-ylmethyl carbonochloridate (643 mg, 1.1 eq.), and the reaction mixture was stirred until complete conversion was observed. After the addition of a 2 M solution of HCl (23 mL) and stirring for 15 min, the reaction mixture was diluted with DCM (100 mL). The organic phase was washed with brine (100 ml), dried, concentrated, and purified by flash chromatography using DCM and MeOH as eluents, to give the title compound (1.1 g, 65%).

¹H NMR (500 MHz, DMSO-d6) δ ppm 11.54 (brs, 1H), 11.07 (brd, 1H), 7.96-6.85 (m, 14H), 6.38-6.08 (s, 1H), 5.44-2.39 (m, 24H), 3.18-3.08 (s, 3H), 2.45-1.90 (s, 3H).

HRMS-ESI (m/z) [M+H]⁺ calcd for C₄₆H₄₇N₄O₆: 751.3490, found 751.3493.

### Step C: 9H-fluoren-9-ylmethyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-7-[(3S)-3-(morpholinomethyl)-3,4-dihydro-1H-isoquinoline-2-carbonyl]-3,4-dihydro-1H-isoquinoline-2-carboxylate

Using General procedure 2b, starting from the product of Step B (1.0 g, 1.3 mmol) as the appropriate acid and 4-[4-[tert-butyl(dimethyl)silyl]oxyanilino]-1,5-dimethyl-pyrrole-2-carbonitrile (548 mg, 1.2 eq.) as the appropriate aniline, the title compound was obtained (1.18 g, 82%).

¹H NMR (400 MHz, DMSO-d6) δ ppm 10.79 (brs, 1H), 8.01-6.45 (m, 19H), 5.45-1.86 (m, 24H), 5.16-4.91 (s, 1H), 3.67-2.96 (s, 6H), 2.46-1.67 (s, 6H), 0.82 (s, 9H), 0.13-0.04 (s, 6H).

HRMS-ESI (m/z) [M+H]⁺ calcd for C₆₅H₇₂N₇O₆Si: 1074.5308, found 1074.5307.

### Step D: N-[4-[tert-butyl(dimethyl)silyl]oxyphenyl]-N-(5-cyano-1,2-dimethyl-pyrrol-3-yl)-1,2-dimethyl-5-[7-[(3S)-3-(morpholinomethyl)-3,4-dihydro-1H-isoquinoline-2-carbonyl]-1,2,3,4-tetrahydroisoquinolin-6-yl]pyrrole-3-carboxamide

The product of Step C (1.1 g, 1.0 mmol) in morpholine (5 mL) and DCM (5 mL) was stirred until complete conversion was observed. The reaction mixture was concentrated and purified by flash chromatography using DCM and MeOH as eluents, to give the title compound (847 mg, 97%).

¹H NMR (500 MHz, DMSO-d6) δ ppm 7.25-6.43 (m, 11H), 5.42-4.98 (s, 1H), 5.13-1.83 (m, 12H), 3.65-3.38 (m, 4H), 3.62-3.02 (s, 6H), 2.57-1.67 (m, 6H), 2.42-1.74 (s, 6H), 0.94-0.79 (s, 9H), 0.16-0.05 (s, 6H).

HRMS-ESI (m/z) [M+H]⁺ calcd for C₅₀H₆₂N₇O₄Si: 852.4624, found 852.4619.

### Step E: N-(5-cyano-1,2-dimethyl-pyrrol-3-yl)-5-[2-[2-[2-fluoro-4-(2-tetrahydropyran-4-ylethoxy)phenyl]acetyl]-7-[(3S)-3-(morpholinomethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

Using General procedure 4c, starting from 2-[2-fluoro-4-(2-tetrahydropyran-4-ylethoxy)phenyl]acetic acid (56 mg, 2.0 eq.) as the appropriate carboxylic acid and the product of Step D (85 mg, 0.10 mmol) as the appropriate amine, the title compound was obtained (37 mg, 37%).

HRMS-ESI (m/z) [M+H]⁺ calcd for C₅₉H₆₅FN₇O₇: 1002.4924, found 1002.4926.

### Example 55: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(thiomorpholin-4-yl)ethoxy]phenyl}acetyl)-1,2,3,4-tetrahydroisoquinolin-6-yl}-1H-pyrrole-3-carboxamide

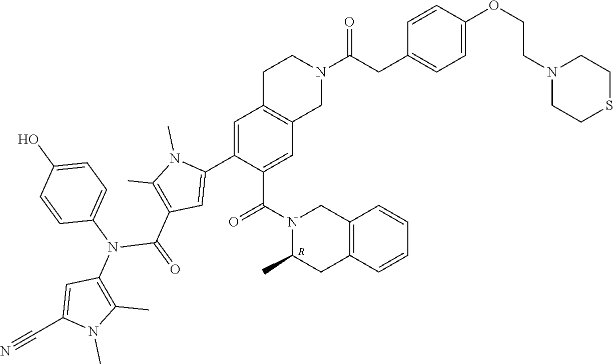

### Step A: methyl 2-[4-(2-thiomorpholinoethoxy)phenyl]acetate

Using the General procedure 7d and then General procedure 8, starting from methyl 2-[4-(2-bromoethoxy)phenyl]acetate (121 mg, 0.44 mmol) as the appropriate alkyl halide and thiomorpholine (55 mg, 3.0 eq.) as the appropriate amine, afforded the title product (88 mg, 53%).

LCMS-ESI (m/z) [M+H]⁺ calcd for C₁₄H₂₀NO₄S: 282.1, found 282.2.

### Step B: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(thiomorpholin-4-yl)ethoxy]phenyl}acetyl)-1,2,3,4-tetrahydroisoquinolin-6-yl}-1H-pyrrole-3-carboxamide

Using General procedure 4c, staring from the product of Step A (83 mg, 1.5 eq.) as the appropriate carboxylic acid and Preparation VIIb (150 mg, 0.20 mmol) as the appropriate amine, afforded the expected product (115 mg, 64%).

HRMS-ESI (m/z) [M+H]⁺ calcd for C₅₄H₅₈N₇O₅S: 916.4215, found 916.4216.

### Example 56: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-((2R)-2-methylmorpholin-4-yl)ethoxy]phenyl}acetyl)-1,2,3,4-tetrahydroisoquinolin-6-yl}-1H-pyrrole-3-carboxamide (Diastereomer 1)

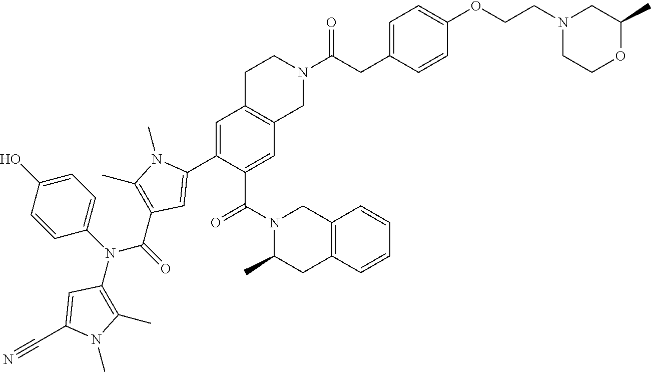 [SMILES: Cc1c(N(C(=O)c2cc(-c3cc4c(cc3C(=O)N3Cc5ccccc5C[C@H]3C)CN(C(=O)Cc3ccc(OCCN5CCO[C@H](C)C5)cc3)CC4)n(C)c2C)c2ccc(O)cc2)cc(C#N)n1C]

### Step A: methyl 2-[4-[2-((2R)-2-methylmorpholin-4-yl)ethoxy]phenyl]acetate and methyl 2-[4-[2-((2S)-2-methylmorpholin-4-yl)ethoxy]phenyl]acetate

Using General procedure 7d, starting from methyl 2-[4-(2-bromoethoxy)phenyl]acetate (320 mg, 1.17 mmol) as the appropriate alkyl halide and 2-methylmorpholine (355 mg, 3 eq.) as the appropriate amine, the desired product was obtained (351 mg, quant.). ¹H NMR (500 MHz, DMSO-d6) δ ppm 7.15 (dm, 2H), 6.88 (dm, 2H), 4.05 (t, 2H), 3.72/3.48 (dm+td, 2H), 3.59 (s, 3H), 3.59 (s, 2H), 3.49 (m, 1H), 2.81/1.78 (d+t, 2H), 2.74/2.08 (d+td, 2H), 2.66 (t, 2H), 1.03 (d, 3H); ¹³C NMR (125 MHz, DMSO-d6) δ ppm 172.4, 157.8, 130.8, 126.8, 114.8, 71.5, 66.3, 65.7, 60.5, 57.3, 53.4, 52.1, 39.7, 19.5.

HRMS-ESI (m/z) [M+H]⁺ calcd for C₁₆H₂₄NO₄: 294.1700, found 294.1702.

Enantiomers of the desired product were separated on an AD-3 chiral column (50×500 mm, 20 m) using 50:50 EtOH-heptane+0.05% DEA as eluents to give the Enantiomer 1 and Enantiomer 2. Enantiomer 1 was identified as (R) and Enantiomer 2 was identified as (S) based on the comparison of the products of test reactions starting from (2R)-2-methylmorpholine and (2S)-2-methylmorpholine.

### Step B: 2-[4-[2-[(2R)-2-methylmorpholin-4-yl]ethoxy]phenyl]acetic acid (from Enantiomer 1)

A solution of Enantiomer 1 (product of Step A) (160 mg, 0.545 mmol) in THE (5.5 mL) and water (2.7 mL) was treated with lithium hydroxide (46 mg, 2 eq.). The reaction mixture was stirred for 18 h. The THF was removed in vacuo and the aqueous residue was acidified with 2 M aq. HCl solution to pH 5.5 then evaporated. Purification by automated flash chromatography using DCM and MeOH (1% TEA) as eluents afforded the title product (184 mg, quant., partial TEA salt).

¹H NMR (500 MHz, DMSO-d6) δ ppm 12.21 (brs, 1H), 7.16 (d, 2H), 6.88 (d, 2H), 4.09 (br, 2H), 3.75/3.51 (m+m, 2H), 3.54 (m, 1H), 3.48 (s, 2H), 2.83/2.72 (m+m, 4H), 2.12/1.81 (m+m, 2H), 1.05 (d, 3H); ¹³C NMR (125 MHz, DMSO-d6) δ ppm 130.9, 114.8, 71.6, 66.2, 65.5, 57.4, 56.7, 40.2, 19.3.

HRMS-ESI (m/z) [M+H]⁺ calcd for C₁₅H₂₂NO₄: 280.1543, found 280.1544.

### Step C. N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-((2R)-2-methylmorpholin-4-yl)ethoxy]phenyl}acetyl)-1,2,3,4-tetrahydroisoquinolin-6-yl}-1H-pyrrole-3-carboxamide (Diastereomer 1)

Using General procedure 4c, starting from the product of Step B (79 mg, 2 eq.) as the appropriate carboxylic acid and Preparation VIIb (80 mg, 0.10 mmol) as the appropriate amine, afforded the desired product (45 mg, 47%).

HRMS-ESI (m/z) [M+H]⁺ calcd for C₅₅H₆₀N₇O₆: 914.4600, found 914.4600.

### Example 57: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-((2S)-2-methylmorpholin-4-yl)ethoxy]phenyl}acetyl)-1,2,3,4-tetrahydroisoquinolin-6-yl}-1H-pyrrole-3-carboxamide (Diastereomer 2)

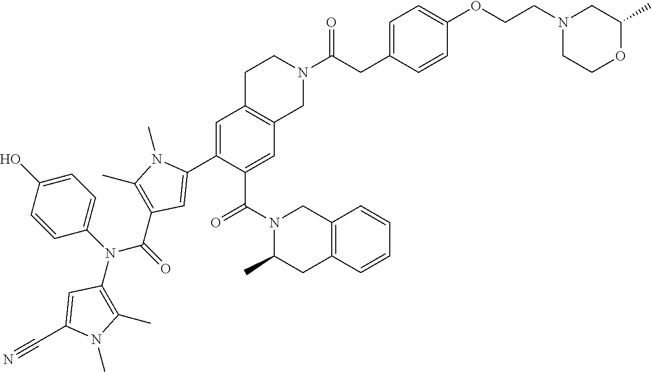 [SMILES: Cc1c(N(C(=O)c2cc(-c3cc4c(cc3C(=O)N3Cc5ccccc5C[C@H]3C)CN(C(=O)Cc3ccc(OCCN5CCO[C@@H](C)C5)cc3)CC4)n(C)c2C)c2ccc(O)cc2)cc(C#N)n1C]

### Step A: 2-[4-[2-[(2S)-2-methylmorpholin-4-yl]ethoxy]phenyl]acetic acid (from Enantiomer 2)

Using General procedure 8, starting from Enantiomer 2 product of Example 56, Step A (167 mg, 0.569 mmol) afforded the title product (150 mg, quant., partial TEA salt).

HRMS-ESI (m/z) [M+H]⁺ calcd for C₁₅H₂₂NO₄: 280.1543, found 280.1545.

### Step B: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-((2S)-2-methylmorpholin-4-yl)ethoxy]phenyl}acetyl)-1,2,3,4-tetrahydroisoquinolin-6-yl}-1H-pyrrole-3-carboxamide (Diastereomer 2)

Using General procedure 4c, starting from the product of Step A (85 mg, 2 eq.) as the appropriate carboxylic acid and Preparation VIIb (80 mg, 0.10 mmol.) as the appropriate amine, afforded the title product (72 mg, 76%).

HRMS-ESI (m/z) [M+H]⁺ calcd for C₅₅H₆₀N₇O₆: 914.4600, found 914.4600.

### Example 58: N-(5-cyano-1,2-dimethylpyrrol-3-yl)-N-(4-hydroxyphenyl)-5-[2-(isoquinolin-3-ylmethyl)-7-{[(3R)-3-methyl-3,4-dihydro-1H-isoquinolin-2-yl]carbonyl}-3,4-dihydro-1H-isoquinolin-6-yl]-1,2-dimethylpyrrole-3-carboxamide

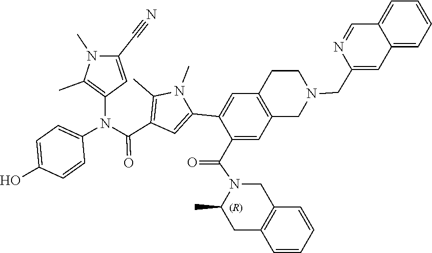

To a solution of Preparation IVa (90 mg, 0.14 mmol, 1 eq.) in DCM (3 mL) was added isoquinoline-3-carboxaldehyde (33 mg, 0.21 mmol, 1.5 eq.) and NaBH(OAc)₃ (58 mg, 0.28 mmol, 2 eq.) and the mixture was stirred at rt for 18 h. The mixture was partitioned between DCM and water. The phases were separated, and the organic phase was dried over MgSO₄ and concentrated in vacuo. Purification by preparative HPLC automated flash chromatography at pH 4 and then at pH 9, eluting with a gradient of 5-95% MeCN in water afforded the title product (16.3 mg, 0.02 mmol, 15%).

HRMS calcd for C₅₀H₄₇N₇O₃: 793.3740, found 794.3817 [M+H]⁺

### Example 59: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{6-[2-(oxan-4-yl)ethoxy]pyridin-3-yl}acetyl)-1,2,3,4-tetrahydroisoquinolin-6-yl}-1H-pyrrole-3-carboxamide

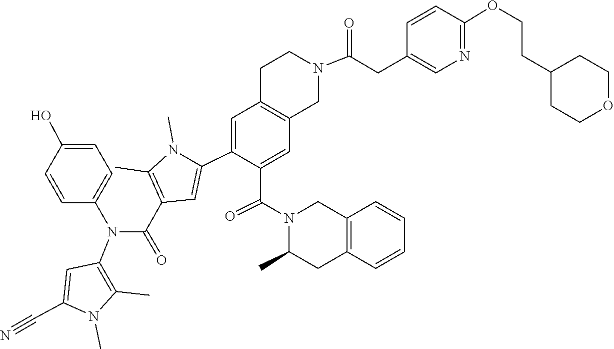 [SMILES: Cc1c(N(C(=O)c2cc(-c3cc4c(cc3C(=O)N3Cc5ccccc5C[C@H]3C)CN(C(=O)Cc3ccc(OCCC5CCOCC5)nc3)CC4)n(C)c2C)c2ccc(O)cc2)cc(C#N)n1C]

### Step A: 2-[6-(2-tetrahydropyran-4-ylethoxy)-3-pyridyl]acetic acid

To a solution of the 2-(6-chloro-3-pyridyl)acetic acid (163 mg, 0.95 mmol) and 2-tetrahydropyran-4-ylethanol (239 mg, 1.9 eq.) in 1,4-dioxane (1.0 mL) was added sodium tert-butoxide (280 mg, 3.0 eq.). Then the mixture was heated to 150° C. and stirred under microwave irradiation until complete conversion was observed. The reaction mixture was purified by HILIC chromatography (using ACN and 5 mM aq. NH₄HCO₃ solution as eluent) to give the title compound (158 mg, 63%).

¹H NMR (500 MHz, DMSO-d6) δ ppm 7.99 (d, 1H), 7.57 (dd, 1H), 6.74 (d, 1H), 4.27 (t, 2H), 3.81/3.27 (dd+td, 4H), 3.51 (s, 2H), 1.65 (m, 1H), 1.64 (q, 2H), 1.60/1.20 (d+dd, 4H); ¹³C NMR (125 MHz, DMSO-d6) δ ppm 173.2, 162.7, 147.4, 140.9, 124.3, 110.5, 67.5, 63.3, 37.3, 35.9, 33.1, 32.0.

HRMS-ESI (m/z) [M+H]⁺ calcd for C₁₄H₂₀NO₄: 266.1387, found 266.1386.

### Step B: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{6-[2-(oxan-4-yl)ethoxy]pyridin-3-yl}acetyl)-1,2,3,4-tetrahydroisoquinolin-6-yl}-1H-pyrrole-3-carboxamide

Using General procedure 4c, starting from the product of Step A (150.0 mg, 4.3 eq.) as the appropriate carboxylic acid and Preparation VIIb (100 mg, 0.13 mmol) as the appropriate amine, afforded the title compound (70.0 mg, 60%).

HRMS-ESI (m/z) [M+H]⁺ calcd for C₅₄H₅₈N₇O₆: 900.4443, found 900.4444.

### Example 60: N-(5-cyano-1,2-dimethyl-1H-pyrrol-3-yl)-N-(4-hydroxyphenyl)-1,2-dimethyl-5-{2-[2-(4-{2-[methyl(oxetan-3-yl)amino]ethoxy}phenyl)acetyl]-7-[(3R)-3-methyl-1,2,3,4-tetrahydroisoquinoline-2-carbonyl]-1,2,3,4-tetrahydroisoquinolin-6-yl}-1H-pyrrole-3-carboxamide

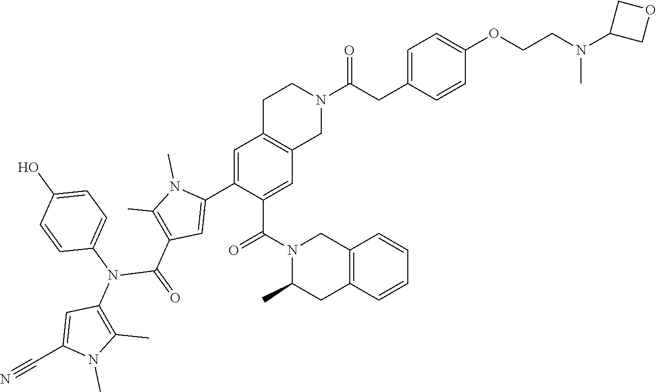 [SMILES: Cc1c(N(C(=O)c2cc(-c3cc4c(cc3C(=O)N3Cc5ccccc5C[C@H]3C)CN(C(=O)Cc3ccc(OCCN(C)C5COC5)cc3)CC4)n(C)c2C)c2ccc(O)cc2)cc(C#N)n1C]

### Step A: 2-[4-[2-[methyl(oxetan-3-yl)amino]ethoxy]phenyl]acetic acid

Using the General procedure 7d and then General procedure 8, starting from methyl 2-[4-(2-bromoethoxy)phenyl]acetate (250 mg, 0.91 mmol) as the appropriate alkyl halide and N-methyloxetan-3-amine (250 mg, 3.1 eq.) as the appropriate amine, the desired product was obtained (180 mg, 90%).

¹H NMR (500 MHz, DMSO-d6) δ ppm 12.08 (br., 1H), 7.16 (m, 2H), 6.87 (m, 2H), 4.53/4.42 (t+t, 4H), 4.00 (t, 2H), 3.65 (br., 1H), 3.48 (s, 2H), 2.63 (br., 2H), 2.17 (s, 3H); ¹³C NMR (125 MHz, DMSO-d6) δ ppm 173.5, 130.9, 114.7, 75.3, 65.7, 59.1, 53.3, 40.2, 38.7.

HRMS-ESI (m/z) [M+H]⁺ calcd for C₁₄H₂₀NO₄: 266.1387, found 266.1387.

### Step B: N-(5-cyano-1,2-dimethyl-1H-pyrrol-3-yl)-N-(4-hydroxyphenyl)-1,2-dimethyl-5-{2-[2-(4-{2-[methyl(oxetan-3-yl)amino]ethoxy]phenyl)acetyl]-7-[(3R)-3-methyl-1,2,3,4-tetrahydroisoquinoline-2-carbonyl]-1,2,3,4-tetrahydroisoquinolin-6-yl}-1H-pyrrole-3-carboxamide

Using General procedure 4c, starting from the product of Step A (170.0 mg, 4.9 eq.) as the appropriate carboxylic acid and Preparation VIIb (100 mg, 0.13 mmol) as the appropriate amine, afforded the title compound (65.0 mg, 55%).

HRMS-ESI (m/z) [M+H]⁺ calcd for C₅₄H₅₈N₇O₆: 900.4448, found 900.4436.

### Example 61: N-(5-cyano-1,2-dimethyl-1H-pyrrol-3-yl)-5-{2-[2-(6-{2-[cyclopropyl(methyl)amino]ethoxy}pyridin-3-yl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

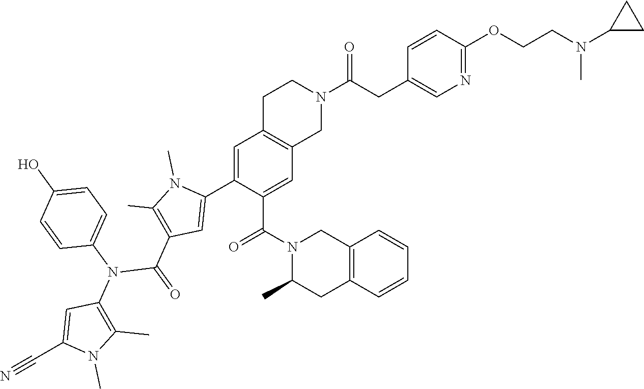 [SMILES: Cc1c(N(C(=O)c2cc(-c3cc4c(cc3C(=O)N3Cc5ccccc5C[C@H]3C)CN(C(=O)Cc3ccc(OCCN(C)C5CC5)nc3)CC4)n(C)c2C)c2ccc(O)cc2)cc(C#N)n1C]

### Step A: 2-[6-[2-[cyclopropyl(methyl)amino]ethoxy]-3-pyridyl]acetic acid

To a solution of the 2-(6-chloro-3-pyridyl)acetic acid (193 mg, 1.12 mmol) and 2-[cyclopropyl(methyl)amino]ethanol (666 mg, 5.1 eq.) in 1,4-dioxane (0.5 mL) was added sodium tert-butoxide (288 mg, 2.7 eq.), then the mixture was heated at 150° C. under microwave irradiation until complete conversion was observed. The reaction mixture was purified by HILIC chromatography (using ACN:NH₄HCO₃ as eluent) to give the title compound (120 mg, 43%).

¹H NMR (500 MHz, DMSO-d6) δ ppm 7.97 (d, 1H), 7.56 (dd, 1H), 6.71 (d, 1H), 4.32 (t, 2H), 3.45 (s, 2H), 2.83 (t, 2H), 2.32 (s, 3H), 1.71 (m, 1H), 0.41/0.28 (m+m, 4H); ¹³C NMR (125 MHz, DMSO-d6) δ ppm 173.4, 162.3, 147.3, 140.8, 125.1, 110.5, 63.5, 56.5, 43.3, 38.7, 38.0, 7.1.

HRMS-ESI (m/z) [M+H]⁺ calcd for C₁₃H₁₈N₂O₃: 251.1390, found 251.1393.

### Step B: N-(5-cyano-1,2-dimethyl-1H-pyrrol-3-yl)-5-{2-[2-(6-{2-[cyclopropyl(methyl)amino]ethoxy]pyridin-3-yl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step A (150.0 mg, 4.6 eq.) as the appropriate carboxylic acid and Preparation VIIb (100 mg, 0.13 mmol) as the appropriate amine, afforded the title compound (71.0 mg, 62%).

HRMS-ESI (m/z) [M+H]⁺ calcd for C₅₃H₅₇N₈O₅: 885.4446, found 885.4447.

### Example 62: N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-{1-methylpyrazolo[3,4-b]pyridin-5-yl}pyrrole-3-carboxamide

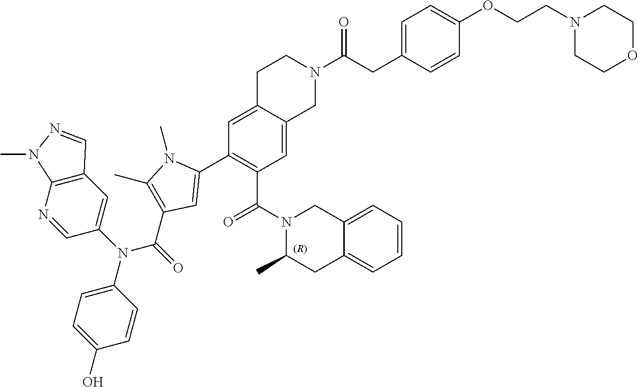

### Step A: N-{4-[(tert-butyldimethylsilyl)oxy]phenyl}-1-methylpyrazolo[3,4-b]pyridin-5-amine

Using General procedure 1a and 5-bromo-1-methyl-1H-pyrazolo[3,4-b]pyridine (1 g, 4.72 mmol, 1 eq.) as the appropriate aryl bromide and Preparation IIa (1.16 g, 5.19 mmol, 1.1 eq.) as the appropriate aniline afforded the title product (732 mg, 2.06 mmol, 44%).

LRMS calcd for C₁₉H₂₆N₄₀Si: 354.2, found 355.2 [M+H]⁺

¹H NMR (400 MHz, DMSO-d6) δ ppm: 8.32 (d, J=2.5 Hz, 1H), 7.96 (s, 1H), 7.92 (s, 1H), 7.71 (d, J=2.5 Hz, 1H), 6.99-6.92 (m, 2H), 6.79-6.73 (m, 2H), 4.01 (s, 3H), 0.95 (s, 9H), 0.17 (s, 6H).

### Step B: N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-{1-methylpyrazolo[3,4-b]pyridin-5-yl}pyrrole-3-carboxamide

Using General procedure 2a and Preparation VIa (175 mg, 0.25 mmol, 1 eq.) as the appropriate acid and the product from Step A (135 mg, 0.38 mmol, 1.5 eq.) as the appropriate aniline afforded an intermediate which was treated according to General procedure 3b to afford the title product (37.4 mg, 0.04 mmol, 16%).

HRMS calcd for C₅₄H₅₆N₈O₆: 912.4323 found, 913.4403 [M+H]⁺

### Example 63: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[(3R or S)-3-(morpholin-4-yl)pyrrolidin-1-yl]phenyl}acetyl)-1,2,3,4-tetrahydroisoquinolin-6-yl}-1H-pyrrole-3-carboxamide (Diastereomer 1)

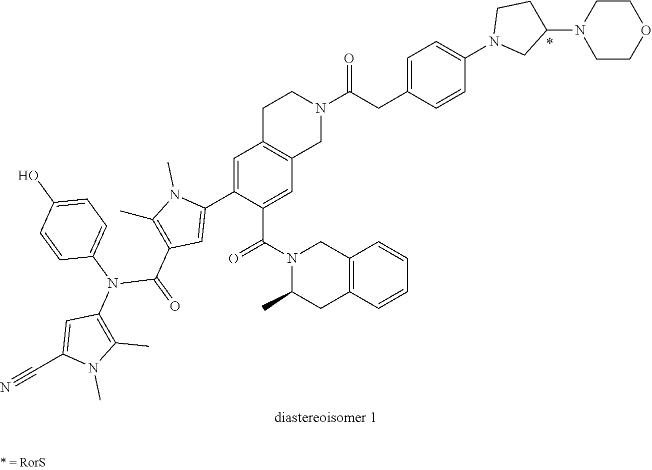 [SMILES: Cc1c(N(C(=O)c2cc(-c3cc4c(cc3C(=O)N3Cc5ccccc5C[C@H]3C)CN(C(=O)Cc3ccc(N5CCC(N6CCOCC6)C5)cc3)CC4)n(C)c2C)c2ccc(O)cc2)cc(C#N)n1C]

### Step A: 2-[4-((3R or S)-3-morpholinopyrrolidin-1-yl)phenyl]acetic acid (Enantiomer 1)

To a degassed solution of methyl 2-(4-bromophenyl)acetate (350 mg, 1.53 mmol) and 4-pyrrolin-3-ylmorpholine (358 mg, 1.5 eq.) in 1,4-dioxane (5 mL/mmol) were added chloro(2-dicyclohexylphosphino-2′,4′,6′-tri-i-propyl-1,1′-biphenyl)[2-(2-aminoethyl)phenyl]palladium(II) [XPhos Palladacycle Gen. 1](113 mg, 0.1 eq.) and Cs₂CO₃ (996 mg, 2 eq.) and the mixture was stirred at 110° C. under N₂ for 2 h. The mixture was allowed to cool to room temperature, was filtered through a pad of Celite and was purified by automated flash chromatography using DCM and methanol as eluents to obtain the racemic product as a brown solid (369 mg, 79%). Enantiomers of the desired product were separated on a Daicel OD chiral column (50×500 mm, 20 m) using 15:85 IPA-heptane+0.05% DEA as eluents to give Enantiomer 1 and Enantiomer 2. Enantiomer 1 was deprotected using General Procedure 8. Purification by automated flash chromatography using DCM and MeOH (1% TEA) as eluents and freeze-drying with hydrochloric acid afforded the HCl salt of the title product as a brown solid (225 mg, quant.).

¹H NMR (500 MHz, DMSO-d6) δ ppm 11.91 (brs, 1H), 7.05 (m, 2H), 6.5 (m, 2H), 3.64 (brs, 4H), 3.44/3.34/3.22/3.09 (brm, 4H), 3.40 (s, 2H), 2.98 (brs, 1H), 2.49 (brs, 4H), 2.18/1.88 (brs, 2H).

HRMS-ESI (m/z) [M+H]⁺ calcd for C₁₆H₂₃N₂O₃: 291.1703, found 291.1704.

### Step B: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[(3R or S)-3-(morpholin-4-yl)pyrrolidin-1-yl]phenyl}acetyl)-1,2,3,4-tetrahydroisoquinolin-6-yl}-1H-pyrrole-3-carboxamide (Diastereomer 1)

Using General procedure 4c, starting from the product of Step A (85 mg, 2.5 eq.) as the appropriate carboxylic acid and Preparation VIIb (80 mg, 0.10 mmol) as the appropriate amine, afforded the title product (64 mg, 66%).

HRMS-ESI (m/z) [M+H]⁺ calcd for C₅₆H₆₁N₈O₅: 925.4759, found 925.4758.

### Example 64: N-(5-cyano-1-isopropyl-2-methyl-pyrrol-3-yl)-N-(4-hydroxyphenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

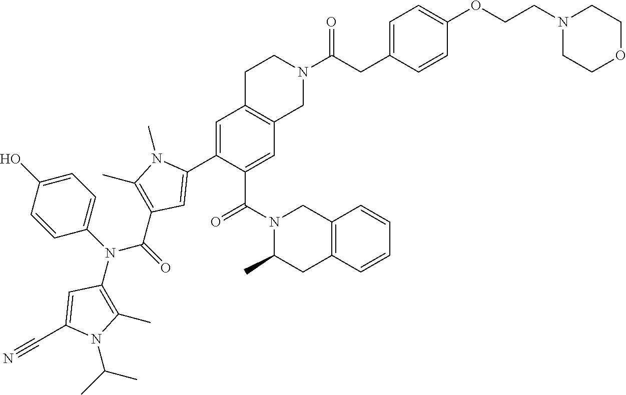 [SMILES: Cc1c(C(=O)N(c2ccc(O)cc2)c2cc(C#N)n(C(C)C)c2C)cc(-c2cc3c(cc2C(=O)N2Cc4ccccc4C[C@H]2C)CN(C(=O)Cc2ccc(OCCN4CCOCC4)cc2)CC3)n1C]

### Step A: 4-bromo-1-isopropyl-5-methyl-pyrrole-2-carbonitrile

Using General procedure 7c, starting from 4-bromo-5-methyl-1H-pyrrole-2-carbonitrile (70 mg, 0.38 mmol) and propan-2-ol (0.3 ml, 2.2 eq) as the appropriate alcohol, afforded the title product (45 mg, 51%).

¹H NMR (500 MHz, DMSO-d6) δ ppm 7.12 (s, 1H), 4.61 (sept., 1H), 2.27 (s, 3H), 1.49 (d, 6H); ¹³C NMR (125 MHz, DMSO-d6) δ ppm 134.1, 122.6, 114.7, 99.7, 95.3, 50.8, 22.3, 11.5.

GC-MS (m/z) [M+H]⁺ calcd for C₉H₁₂BrN₂: 227, found 226/228.

### Step B: 4-[4-[tert-butyl(dimethyl)silyl]oxyanilino]-1-isopropyl-5-methyl-pyrrole-2-carbonitrile

Using General procedure 1b, starting from the product of Step A (40 mg, 0.18 mmol) as the appropriate aryl bromide and Preparation IIIa (59 mg, 1.5 eq.) as the appropriate aniline, afforded the title product (41 mg, 63%).

¹H NMR (500 MHz, DMSO-d6) δ ppm 6.83 (s, 1H), 6.78 (s, 1H), 6.60 (d, 2H), 6.49 (d, 2H), 4.56 (m, 1H), 2.12 (s, 3H), 1.51 (d, 6H), 0.92 (s, 9H), 0.11 (s, 6H); ¹³C NMR (125 MHz, DMSO-d6) δ ppm 146.7, 142.6, 130.2, 124.5, 120.6, 117.7, 116, 114.3, 96.5, 49.5, 26.1, 22.5, 18.4, 10.2, −4.0.

HPLC-MS-ESI [M+H]⁺ calcd for C₂₁H₃₂N₃OSi: 370.2, found 370.2.

### Step C. N-[4-[tert-butyl(dimethyl)silyl]oxyphenyl]-N-(5-cyano-1-isopropyl-2-methyl-pyrrol-3-yl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

The title compound was obtained using the method described in Preparation VIIb and the product from Step B as the appropriate aniline for the amide formation.

¹H NMR (500 MHz, DMSO-d6) δ ppm 7.30-6.36 (m, 11H), 5.51-4.89 (s, 1H), 5.40-1.80 (m, 12H), 4.50 (brm, 1H), 3.50-2.99 (s, 3H), 2.44-1.80 (s, 6H), 1.46 (brd, 6H), 1.08-0.44 (d, 3H), 0.84 (s, 9H), −0.04 (s, 6H).

HPLC-ESI-MS (m/z) [M+H]⁺ calcd for C₄₈H₅₉N₆O₃Si: 795.4, found 795.4.

### Step D: N-(5-cyano-1-isopropyl-2-methyl-pyrrol-3-yl)-N-(4-hydroxyphenyl)-1,2-dimethyl-5-[7-[(3R)-3-methyl-3,4-dihydro-1H-isoquinoline-2-carbonyl-2-[2-[4-(2-morpholinoethoxy)phenyl]acetyl]-3,4-dihydro-1H-isoquinolin-6-yl]pyrrole-3-carboxamide

Using General procedure 4c, starting from Preparation Va as the appropriate acid (46 mg, 0.157 mmol, 2.5 eq.) and the product of Step C (50 mg, 0.063 mmol, 1 eq.) as the appropriate amine, the desired product was obtained (24.7 mg, 42%).

HRMS-ESI (m/z) [M+H]⁺ calcd for C₅₆H₆₂N₇O₆: 928.4756, found 928.4755.

### Example 65: N-(5-cyano-1,2-dimethylpyrrol-3-yl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-phenylpyrrole-3-carboxamide

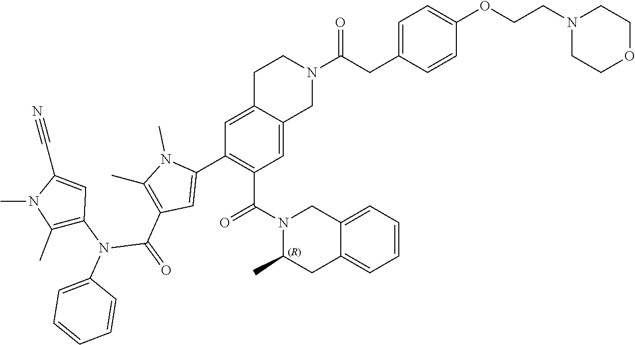

### Step A: 1,5-dimethyl-4-(phenylamino)pyrrole-2-carbonitrile

Using General procedure 1a and the product from Preparation IVa, Step A (10 g, 50.24 mmol, 1 eq.) as the appropriate aryl bromide and aniline (5.5 mL, 60.29 mmol, 1.2 eq.) afforded the title product (6.36 g, 30.12 mmol, 60%).

LRMS calcd for C₁₃H₁₃N₃: 211.1, found 212.0 [M+H]⁺

¹H NMR (400 MHz, DMSO-d6) δ ppm: 7.17 (s, 1H), 7.10-7.02 (m, 2H), 6.77 (s, 1H), 6.61-6.54 (m, 3H), 3.63 (s, 3H), 2.09 (s, 3H).

### Step B: N-(5-cyano-1,2-dimethylpyrrol-3-yl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-phenylpyrrole-3-carboxamide

Using General procedure 2a and Preparation VIa (163 mg, 0.24 mmol, 1 eq.) as the appropriate acid and the product from Step A (75 mg, 0.35 mmol, 1.5 eq.) as the appropriate aniline, followed with a purification step by preparative HPLC automated flash chromatography at pH 9 eluting with a gradient of 5-95% MeCN in water afforded the title product (10.3 mg, 0.01 mmol, 5%).

HRMS calcd for C₅₄H₅₇N₇O₅: 883.4421, found 884.4511 [M+H]⁺.

### Example 66: N-(5-cyano-1,2-dimethylpyrrol-3-yl)-N-(4-hydroxyphenyl)-1,2-dimethyl-5-[2-(2-{4-[2-(oxan-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]pyrrole-3-carboxamide

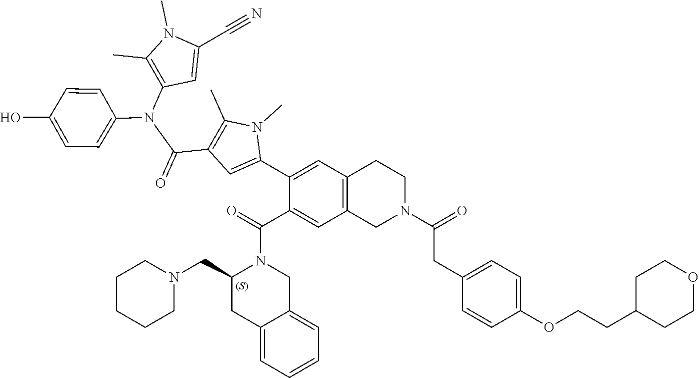

Using General procedure 4a and Preparation IVb (142 mg, 0.19 mmol, 1 eq.) as the appropriate amine and Preparation Vc (56 mg, 0.21 mmol, 1.1 eq.) as the appropriate carboxylic acid afforded the title product (49.7 mg, 0.05 mmol, 26%).

HRMS calcd for C₆₀H₆₇N₇O₆: 981.5153, found 982.5237 [M+H]⁺

### Example 67: N-(5-cyano-1,2-dimethylpyrrol-3-yl)-N-(4-hydroxyphenyl)-1,2-dimethyl-5-[2-(2-{4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]pyrrole-3-carboxamide

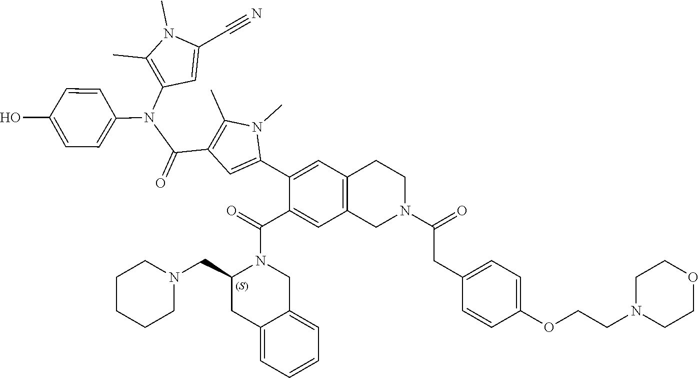

Using General procedure 4a and Preparation IVb (142 mg, 0.19 mmol, 1 eq.) as the appropriate amine and Preparation Va (56 mg, 0.21 mmol, 1.1 eq.) as the appropriate carboxylic acid afforded the title product (71 mg, 0.07 mmol, 37%).

HRMS calcd for C₅₉H₆₆N₈O₆: 982.5105, found 983.5185 [M+H]⁺

### Example 68: N-[5-cyano-1-(2-methoxyethyl)-2-methyl-pyrrol-3-yl]-N-(4-hydroxyphenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

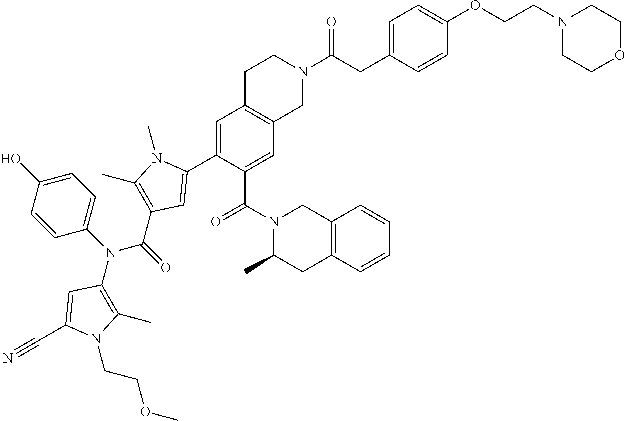 [SMILES: COCCn1c(C#N)cc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(O)cc2)c1C]

### Step A: 4-bromo-1-(2-methoxyethyl)-5-methyl-pyrrole-2-carbonitrile

Using General procedure 7c, starting from 4-bromo-5-methyl-1H-pyrrole-2-carbonitrile (100 mg, 0.54 mmol) and 2-methoxyethanol (80 mg, 2 eq) as the appropriate alcohol, afforded the title product (100 mg, 71%).

¹H NMR (500 MHz, DMSO-d6) δ ppm 7.07 (s, 1H), 4.20 (t, 2H), 3.56 (t, 2H), 3.22 (s, 3H), 2.24 (s, 3H); ¹³C NMR (125 MHz, DMSO-d6) δ ppm 134.8, 120.8, 113.7, 103.4, 95.4, 71.1, 58.8, 47.2, 11.2.

GC-MS (m/z) [M+H]⁺ calcd for C₉H₁₂BrN₂O: 243, found 243.

### Step B: 4-[4-[tert-butyl(dimethyl)silyl]oxyanilino]-1-(2-methoxyethyl)-5-methyl-pyrrole-2-carbonitrile

Using General procedure 1b, starting the product of Step A (100 mg, 0.41 mmol) as the appropriate aryl bromide and Preparation IIIa (138 mg, 1.5 eq.) as the appropriate aniline, afforded the title product (120 mg, 76%).

¹H NMR (500 MHz, DMSO-d6) δ ppm 6.88 (s, 1H), 6.75 (s, 1H), 6.60 (dm, 2H), 6.49 (dm, 2H), 4.15 (t, 2H), 3.59 (t, 2H), 3.24 (s, 3H), 2.10 (s, 3H), 0.92 (s, 9H), 0.12 (s, 6H); ¹³C NMR (125 MHz, DMSO-d6) δ ppm 146.7, 142.5, 130.7, 124.8, 120.6, 115.8, 114.9, 114.2, 100.1, 71.4, 58.8, 46.2, 26.1, 9.9, −4.1.

HRMS-ESI (m/z) [M+H]⁺ calcd for C₂₁H₃₂N₃O₂Si: 386.2259, found 386.2259.

### Step C: N-[4-[tert-butyl(dimethyl)silyl]oxyphenyl]-N-[5-cyano-1-(2-methoxyethyl)-2-methyl-pyrrol-3-yl]-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

The title compound was obtained using the method described in Preparation VIIb and the product from Step B as the appropriate aniline for the amide formation.

¹H NMR (500 MHz, DMSO-d6) δ ppm 9.47-9.27 (s, 1H), 7.28-6.40 (m, 11H), 5.54-5.09 (s, 1H), 5.35-1.95 (m, 16H), 3.41-3.01 (s, 3H), 3.12-3.02 (s, 3H), 2.45-1.80 (s, 6H), 1.09-0.42 (d, 3H).

HRMS-ESI (m/z) [M+H]⁺ calcd for C₄₂H₄₅N₆O₄: 697.3497, found 697.3496.

### Step D: N-[5-cyano-1-(2-methoxyethyl)-2-methyl-pyrrol-3-yl]-N-(4-hydroxyphenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

Using General procedure 4c, starting from Preparation Va as the appropriate acid (41 mg, 2.5 eq.) and the product of Step C (45 mg, 0.06 mmol) as the appropriate amine, the desired product was obtained (15 mg, 29%).

HRMS-ESI (m/z) [M+H]⁺ calcd for C₅₆H₆₂N₇O₇: 944.4705, found 944.4706.

### Example 69: N-(5-cyano-1,2-dimethylpyrrol-3-yl)-5-[2-(2-{2,5-difluoro-4-[2-(morpholin-4-yl)ethoxy]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

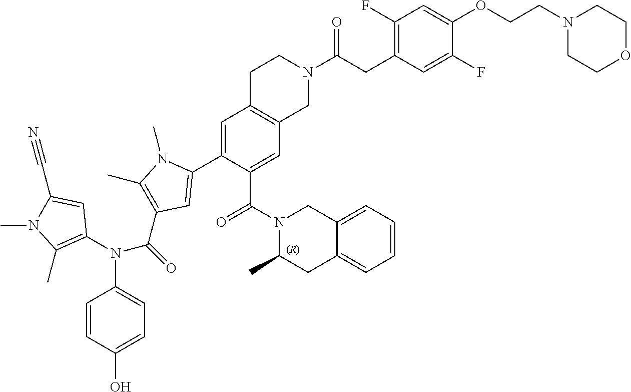

### Step A: ethyl 2-(2,5-difluoro-4-methoxyphenyl)acetate

A vessel containing a 0.6 M solution of ethyl bromoacetate (17.1 mL, 154 mmol, 3 eq.) in THE (250 mL) was placed under N₂.

A cartridge supplied by OmniFit™ was loaded with zinc granules mesh 300 (12 g excess).

A second vessel containing a 0.25 M solution of 4-bromo-2,5-difluoroanisole in THE (200 mL, 49.32 mmol, 1 eq.) was placed under N₂. To this solution was added X-Phos (0.59 g, 1.23 mmol, 0.03 eq.) and Pd₂dba₃ (0.56 g, 0.62 mmol, 0.01 eq.) and the mixture sparged with N₂ for 20 mins and then sonicated to form a homogenous mixture.

A Syrris Asia Flow Chemistry System™ was set up with a column heater into which the cartridge containing the zinc was mounted and heated to 40° C. The zinc was pre-activated using a procedure analogous to the method according to Berton, M., Huck, L. & Alcázar, J. On-demand synthesis of organozinc halides under continuous flow conditions. Nat Protoc 13, 324-334 (2018).

A T-mixer (head-to-head) was placed prior to the tube reactor and reagent feeds to the Asia pumps were set up under N₂ in the Asia Reagent Store. A 16 mL fluoropolymer tube reactor was mounted on an Asia heater module and was surrounded by 450 nm (blue) LED lights (18 W). The tube heater was set to 80° C.

The system back pressure regulator was set to 2 bar. The solution of ethyl bromoacetate flow rate was set to 250 μL min⁻¹. The 4-bromo-2,5-difluoroanisole solution flow rate was set at 250 μL min⁻¹ and the resultant combined flow rate in the tube reactor was 500 μL min⁻¹ giving a reaction residence time of 32 mins.

The collected crude product solution was passed through a celite cartridge to remove spent palladium and washed through with further THF. The combined filtrate was diluted with EtOAc, washed with sat. aq. NH₄Cl solution, brine and then dried over MgSO₄ and concentrated in vacuo. Purification by automated flash chromatography eluting with a gradient of heptane-42% DCM in heptane afforded the title product (10.9 g, 47 mmol, 95%).

LRMS calcd for C₁₁H₁₂F₂O₃: 230.1, found 231.2 [M+H]⁺

¹H NMR (400 MHz, DMSO-d6) δ ppm: 7.24 (dd, J=11.8, 7.1 Hz, 1H), 7.11 (dd, J=11.2, 7.4 Hz, 1H), 4.08 (q, J=7.1 Hz, 2H), 3.83 (s, 3H), 3.63 (d, J=1.5 Hz, 2H), 1.18 (t, J=7.1 Hz, 3H).

### Step B: ethyl 2-(2,5-difluoro-4-hydroxyphenyl)acetate

A solution of 1 M BBr₃ in DCM (14.53 mL, 14.53 mmol, 1.5 eq.) was added dropwise over 15 mins to a suspension of the product from Step A (2.23 g, 9.69 mmol, 1 eq.) in anhydrous DCM (40 mL), cooled to −78° C. under N₂. The mixture was allowed to warm to rt and stirred for 3 h. 100 g of crushed ice was added and the mixture was stirred vigorously for 15 mins. The DCM was removed in vacuo and the aqueous was extracted with EtOAc. The organic phase was washed with water, brine, dried over MgSO₄ and concentrated in vacuo. Purification by automated flash chromatography eluting with a gradient of 0-50% EtOAc in heptane afforded the title product (1.22 g, 5.64 mmol, 58%).

LRMS calcd for C₁₀H10F₂O₃: 216.1, found 215.0 [M−H]⁻

¹H NMR (400 MHz, DMSO-d6) δ ppm: 10.28 (s, 1H), 7.15 (dd, J=11.5, 7.2 Hz, 1H), 6.74 (dd, J=10.8, 7.4 Hz, 1H), 4.08 (q, J=7.1 Hz, 2H), 3.57 (d, J=1.4 Hz, 2H), 1.17 (t, J=7.1 Hz, 3H).

### Step C: ethyl 2-{2,5-difluoro-4-[2-(morpholin-4-yl)ethoxy]phenyl]acetate

4-(2-Chloroethyl)morpholine hydrochloride (2.25 g, 12.07 mmol, 2 eq.) was added to a mixture of the product from Step B (1.22 g, 6.03 mmol, 1 eq.) and KI (0.1 g, 0.6 mmol, 0.1 eq.) in acetone. K₂CO₃ (4.17 g, 30.17 mmol, 5 eq.) was added and the mixture was heated at 60° C. for 2 days. The mixture was allowed to cool to rt and then concentrated in vacuo. The residue was partitioned between EtOAc and water. The phases were separated, and the organic phase was washed with brine, dried over MgSO₄ and concentrated in vacuo. Purification by automated flash chromatography eluting with a gradient of 0-9% MeOH in DCM afforded the title product (1.46 g, 4.63 mmol, 77%).

¹H NMR (400 MHz, DMSO-d6) δ ppm: 7.23 (dd, J=11.7, 7.1 Hz, 1H), 7.15 (dd, J=11.3, 7.3 Hz, 1H), 4.16 (t, J=5.7 Hz, 2H), 4.08 (q, J=7.1 Hz, 2H), 3.62 (d, J=1.4 Hz, 2H), 3.59-3.52 (m, 4H), 2.70 (t, J=5.7 Hz, 2H), 2.49-2.44 (m, 4H), 1.18 (t, J=7.1 Hz, 3H).

### Step D: [2,5-difluoro-4-[2-(morpholin-4-yl)ethoxy]phenyl}acetic acid

Using General procedure 8 and the product from Step C (1.46 g, 4.63 mmol, 1 eq.) as the appropriate ester afforded the title product (621 mg, 2.06 mmol, 45%).

LRMS calcd for C₁₄H₁₇F₂NO₄: 301.1, found 302.2 [M+H]⁺

¹H NMR (400 MHz, DMSO-d6) δ ppm: 7.12 (dd, J=12.1, 7.1 Hz, 1H), 7.00 (dd, J=11.1, 7.4 Hz, 1H), 4.12 (t, J=5.7 Hz, 2H), 3.60-3.53 (m, 4H), 3.20 (d, J=1.5 Hz, 2H), 2.68 (t, J=5.7 Hz, 2H), 2.49-2.43 (m, 4H).

### Step E: N-(5-cyano-1,2-dimethylpyrrol-3-yl)-5-[2-(2-[2,5-difluoro-4-[2-(morpholin-4-yl)ethoxy]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and Preparation IVa (132 mg, 0.2 mmol, 1 eq.) as the appropriate amine and the product from Step D (73 mg, 0.24 mmol, 1.2 eq.) as the appropriate carboxylic acid afforded the title product (51 mg, 0.05 mmol, 27%).

HRMS calcd for C₅₄H₅₅F₂N₇O₆: 935.4182, found 936.4262 [M+H]⁺

### Example 70: N-(5-cyano-1,2-dimethyl-pyrrol-3-yl)-5-[2-[2-[2-fluoro-4-[(1S,2R) or (1R,2S)-2-morpholinocyclopropyl]phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereomer 1)

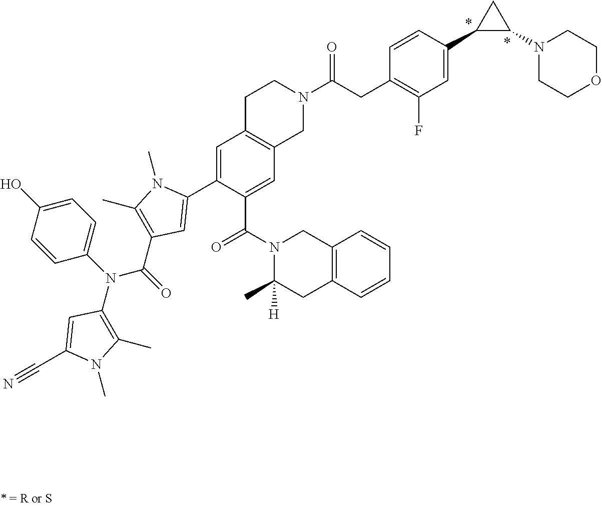 [SMILES: Cc1c(N(C(=O)c2cc(-c3cc4c(cc3C(=O)N3Cc5ccccc5C[C@H]3C)CN(C(=O)Cc3ccc([C@H]5C[C@@H]5N5CCOCC5)cc3F)CC4)n(C)c2C)c2ccc(O)cc2)cc(C#N)n1C]

### Step A: trans-4-[2-(4-bromo-3-fluorophenyl)cyclopropyl]morpholine (mixture of (1S,2R) and (1R,2S) stereoisomers)

1-bromo-2-(2-bromoethoxy)ethane (0.39 mL, 3.1 mmol, 1.1 eq.) was added to suspension of trans-2-(4-bromo-3-fluorophenyl)cyclopropan-1-amine hydrochloride (750 mg, 2.81 mmol, 1 eq.) and K₂CO₃ (1.17 g, 8.44 mmol, 3 eq.) in MeCN (10 mL) and the mixture stirred at 60° C. under N₂ for 18 h. The mixture was allowed to cool to rt and partitioned between DCM and brine. The phases were separated, and the organic phase was dried (PTFE phase separator) and concentrated in vacuo. Purification by automated flash chromatography eluting with a gradient of 0-70% EtOAc in heptane afforded the title product (469 mg, 56%).

LRMS calcd for C₁₃H₁₅BrFNO: 299.0, found 300.2 [M+H]⁺

¹H NMR (400 MHz, DMSO-d6) δ ppm: 7.53 (dd, J=8.3, 7.6 Hz, 1H), 7.11 (dd, J=10.5, 2.1 Hz, 1H), 6.92 (dd, J=8.3, 2.1 Hz, 1H), 3.60-3.47 (m, 4H), 2.58-2.47 (m, 4H), 2.04-1.96 (m, 1H), 1.91-1.84 (m, 1H), 1.12-1.01 (m, 2H).

### Step B: ethyl 2-{2-fluoro-4-[(1S,2R)-2-(morpholin-4-yl)cyclopropyl]phenyl}acetate and ethyl 2-{2-fluoro-4-[(1R,2S)-2-(morpholin-4-yl)cyclopropyl]phenyl]acetate

Using the procedure described in Example 69, Step A and a solution of the product from Step A in THE (0.25 M, 6.22 mL, 1.56 mmol, 1 eq.), followed with a purification step by automated flash chromatography eluting with a gradient of 0-100% EtOAc in heptane afforded the title product (144 mg, 30%).

LRMS calcd for C₁₇H₂₂FNO₃: 307.2, found 308.2 [M+H]⁺

¹H NMR (400 MHz, DMSO-d6) δ ppm: 7.21-7.15 (m, 1H), 6.93-6.86 (m, 2H), 4.07 (q, J=7.1 Hz, 2H), 3.65-3.61 (m, 2H), 3.59-3.48 (m, 4H), 2.59-2.49 (m, 4H), 2.01-1.92 (m, 1H), 1.90-1.82 (m, 1H), 1.17 (t, J=7.1 Hz, 3H), 1.09-0.96 (m, 2H).

Stereomers of the desired product were separated on an OD chiral column (50×500 mm, 20 m) using 15:85 2-propanol/heptane+0.05% DEA as eluents to give Stereoisomer 1 and Stereoisomer 2.

### Step C: 2-[2-fluoro-4-[(1S,2R) or (1R,2S)-2-morpholinocyclopropyl]phenyl]acetic acid (from Stereoisomer 1)

Using the General procedure 8, starting from the Enantiomer 1 of Step B (51 mg, 0.166 mmol), the desired product was obtained (44 mg, 95%).

¹H NMR (500 MHz, DMSO-d6) δ ppm 12 (brs, 1H), 7.15 (t, 1H), 6.87 (dd, 1H), 6.86 (m, 1H), 3.53 (m, 4H), 3.49 (s, 2H), 2.54 (m, 4H), 1.95 (m, 1H), 1.84 (m, 1H), 1.04/1 (m+m, 2H); ¹³C NMR (125 MHz, DMSO-d6) δ ppm 172.5, 132, 121.9, 112.6, 66.6, 53.3, 49.8, 34.9, 23.7, 16.5.

HRMS-ESI (m/z) [M+H]⁺ calcd for C₁₅H₁₉FNO₃: 280.1343, found 280.1343.

### Step D: N-(5-cyano-1,2-dimethyl-pyrrol-3-yl)-5-[2-[2-[2-fluoro-4-[(1S,2R) or (1R,2S)-2-morpholinocyclopropyl]phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereomer 1)

Using General procedure 4c, starting from the product of Step C (44 mg, 1.5 eq.) as the appropriate carboxylic acid and Preparation VIIb (79 mg, 0.102 mmol) as the appropriate amine, afforded the title compound (29.0 mg, 20%).

HRMS-ESI (m/z) [M+H]⁺ calcd for C₅₅H₅₇FN₇O₅: 914.4400, found 914.4408.

### Example 71: N-(5-cyano-1-cyclopropyl-2-methyl-pyrrol-3-yl)-N-(4-hydroxyphenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

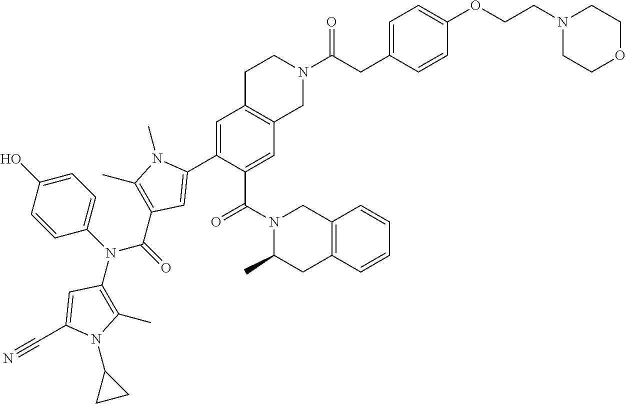 [SMILES: Cc1c(C(=O)N(c2ccc(O)cc2)c2cc(C#N)n(C3CC3)c2C)cc(-c2cc3c(cc2C(=O)N2Cc4ccccc4C[C@H]2C)CN(C(=O)Cc2ccc(OCCN4CCOCC4)cc2)CC3)n1C]

### Step A: 4-bromo-1-cyclopropyl-5-methyl-pyrrole-2-carbonitrile

A 4 mL screw-cup vial was charged with 4-bromo-5-methyl-1H-pyrrole-2-carbonitrile (200 mg, 1.08 mmol), bromocyclopropane (326.9 mg, 2.5 eq) and cesium carbonate (2.7 g, 2 eq.) in DMF (2 mL/mmol). The reaction mixture was stirred at 160° C. under N₂ for 24 hours. The volatiles of the reaction mixture were evaporated, the residue was dissolved in DCM (5 mL), and it was concentrated to Celite. Purification via automated flash chromatography, using DCM and EtOAc as eluents afforded the title compound (124 mg, 51%).

¹H NMR (500 MHz, DMSO-d6) δ ppm 7.05 (s, 1H), 3.35 (m, 1H), 1.15-1.00 (m, 4H); ¹³C NMR (125 MHz, DMSO-d6) δ ppm 136.8, 121.1, 113.8, 104.3, 95.3, 28.7, 11.7, 7.5.

GC-MS (m/z) [M+2H]⁺ calcd for C₉H₁₁BrN₂: 226, found 226.

### Step B: 4-[4-[tert-butyl(dimethyl)silyl]oxyanilino]-1-cyclopropyl-5-methyl-pyrrole-2-carbonitrile

Using General procedure 1b and the product of Step A (120 mg, 0.533 mmol) as the appropriate aryl bromide and Preparation IIa (178 mg, 1.5 eq.) as the appropriate aniline, afforded the title product (102 mg, 52%).

¹H NMR (500 MHz, DMSO-d6) δ ppm 6.87 (s, 1H), 6.72 (s, 1H), 6.60 (d, 2H), 6.51 (d, 2H), 3.29 (m, 1H), 2.15 (s, 3H), 1.09/1.02 (m+m, 4H), 0.92 (s, 9H), 0.11 (s, 6H); ¹³C NMR (125 MHz, DMSO-d6) δ ppm 146.7, 142.4, 132.3, 124.8, 120.6, 116.1, 115.1, 114.3, 101.2, 27.9, 26.1, 18.4, 10.4, 7.4, −4.0.

HPLC-MS-ESI (m/z) [M+H]⁺ calcd for C₂₁H₃₀N₃OSi: 368.2, found 368.2.

### Step C. N-[4-[tert-butyl(dimethyl)silyl]oxyphenyl]-N-(5-cyano-1-cyclopropyl-2-methyl-pyrrol-3-yl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

The title compound was obtained using the method described in Preparation VIIb and the product from Step B as the appropriate aniline for the amide formation.

HPLC-MS-ESI (m/z) [M+H]⁺ calcd for C₄₈H₅₇N₆O₃Si: 793.4, found 793.4.

### Step D: N-(5-cyano-1-cyclopropyl-2-methyl-pyrrol-3-yl)-N-(4-hydroxyphenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

Using General procedure 4c, starting from Preparation Va as the appropriate acid (45 mg, 2.5 eq.) and the product of Step C (48 mg, 0.06 mmol) as the appropriate amine, the desired product was obtained (8 mg, 14%).

HRMS-ESI (m/z) [M+H]⁺ calcd for C₅₆H₆₀N₇O₆: 926.4600, found 926.4606.

### Example 72: N-(5-fluoro-6-methoxypyridin-3-yl)-N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxamide

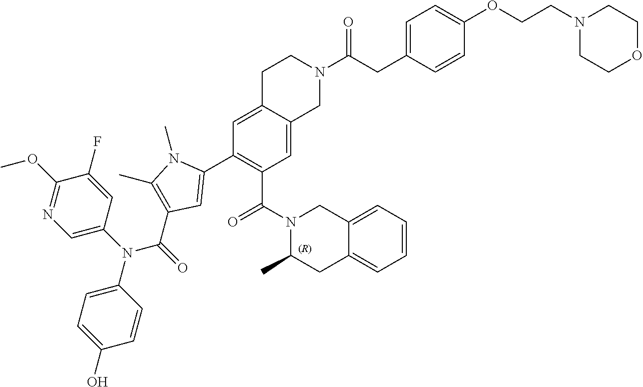

### Step A: N-{4-[(tert-butyldimethylsilyl)oxy]phenyl}-5-fluoro-6-methoxypyridin-3-amine

Using General procedure 1a and 5-bromo-3-fluoro-2-methoxypyridine (1 g, 4.85 mmol, 1 eq.) as the appropriate aryl bromide and Preparation IIa (1.14 g, 5.1 mmol, 1.05 eq.) as the appropriate aniline afforded the title product (1.1 g, 3.16 mmol, 65%).

LRMS calcd for C₁₈H₂₅FN₂O₂ Si: 348.2, found 349.2 [M+H]⁺

¹H NMR (400 MHz, DMSO-d6) δ ppm: 7.87 (s, 1H), 7.67 (d, J=2.4 Hz, 1H), 7.30 (dd, J=12.4, 2.4 Hz, 1H), 6.95-6.88 (m, 2H), 6.78-6.71 (m, 2H), 3.86 (s, 3H), 0.94 (s, 9H), 0.16 (s, 6H).

### Step B: N-(5-fluoro-6-methoxypyridin-3-yl)-N-(4-hydroxyphenyl)-1,2-dimethyl-5-(7-{[(3R)-3-methyl-3,4-dihydro-1H-isoquinolin-2-yl]carbonyl]-2-(2-{4-[2-(morpholin-4-yl)ethoxy]phenyl]acetyl)-3,4-dihydro-1H-isoquinolin-6-yl)pyrrole-3-carboxamide

Using General procedure 2a and Preparation VIa (175 mg, 0.25 mmol, 1 eq.) as the appropriate acid and the product from Step A (132 mg, 0.38 mmol, 1.5 eq.) as the appropriate aniline afforded an intermediate which was treated according to General procedure 3b to afford the title product (17 mg, 0.02 mmol, 7%).

HRMS calcd for C₅₃H₅₅FN₆O₇: 906.4116, found 907.4208 [M+H]⁺

### Example 73: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-{2-[4-(2-{6-oxa-9-azaspiro[4.5]decan-9-yl}ethoxy)phenyl]acetyl}-1,2,3,4-tetrahydroisoquinolin-6-yl}-1H-pyrrole-3-carboxamide

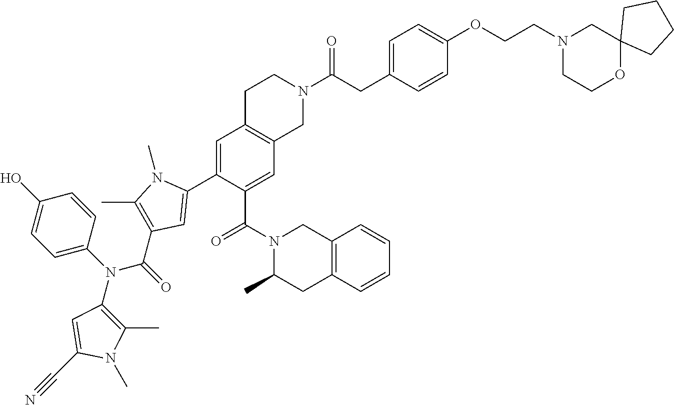 [SMILES: Cc1c(N(C(=O)c2cc(-c3cc4c(cc3C(=O)N3Cc5ccccc5C[C@H]3C)CN(C(=O)Cc3ccc(OCCN5CCOC6(CCCC6)C5)cc3)CC4)n(C)c2C)c2ccc(O)cc2)cc(C#N)n1C]

### Step A: 2-[4-[2-(6-oxa-9-azaspiro[4.5]decan-9-yl)ethoxy]phenyl]acetic acid

Using the General procedure 7d and then General procedure 8, starting from methyl 2-[4-(2-bromoethoxy)phenyl]acetate (150 mg, 0.55 mmol) as the appropriate alkyl halide and 6-oxa-9-azaspiro[4.5]decane (155 mg, 2.0 eq.) as the appropriate amine, the desired product was obtained (169 mg, 96%).

¹H NMR (500 MHz, DMSO-d6) δ ppm 11.97 (brs, 1H), 7.17 (m, 2H), 6.89 (m, 2H), 4.08 (brs, 2H), 3.58 (brs, 2H), 3.48 (s, 2H), 2.66 (brs, 2H), 2.43 (brs, 2H), 2.36 (brs, 2H), 1.80-1.42 (brm, 8H).

HRMS-ESI (m/z) [M+H]⁺ calcd for C18H₂₆NO₄: 320.1856, found 320.1858.

### Step B: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-{2-[4-(2-{6-oxa-9-azaspiro[4.5]decan-9-yl}ethoxy)phenyl]acetyl}-1,2,3,4-tetrahydroisoquinolin-6-yl}-1H-pyrrole-3-carboxamide

Using General procedure 4c, starting from the product of Step A (67.0 mg, 2.0 eq.) as the appropriate carboxylic acid and Preparation VIIb (80 mg, 0.10 mmol) as the appropriate amine, afforded the title compound (70.0 mg, 70%).

HRMS-ESI (m/z) [M+H]⁺ calcd for C₅₈H₆₄N₇O₆: 954.4918, found 954.4915.

### Example 74: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-[2-(piperidin-1-yl)ethyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

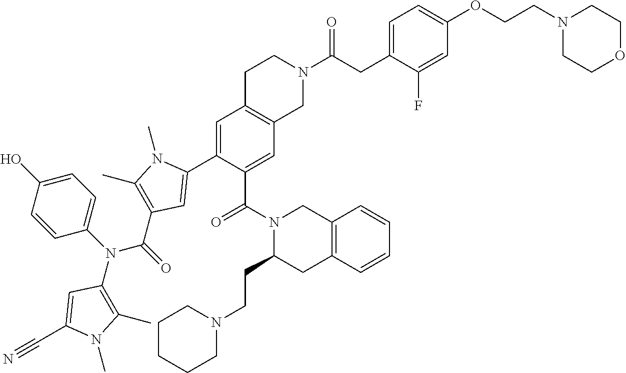 [SMILES: Cc1c(N(C(=O)c2cc(-c3cc4c(cc3C(=O)N3Cc5ccccc5C[C@H]3CCN3CCCCC3)CN(C(=O)Cc3ccc(OCCN5CCOCC5)cc3F)CC4)n(C)c2C)c2ccc(O)cc2)cc(C#N)n1C]

### Step A: tert-butyl (3S)-3-[2-oxo-2-(1-piperidyl)ethyl]-3,4-dihydro-1H-isoquinoline-2-carboxylate

Using General procedure 5a, starting from 2-[(3S)-2-tert-butoxycarbonyl-3,4-dihydro-]H-isoquinolin-3-yl]acetic acid (300 mg, 1.03 mmol) as the appropriate carboxylic acid and piperidine (123 mg, 1.4 eq.) as the appropriate amine, followed with a purification step via automated flash chromatography with DCM and methanol as eluents provided the desired compound (332 mg, 90%).

¹H NMR (500 MHz, DMSO-d6) δ ppm 7.25-7.09 (m, 4H), 4.79-4.59 (br., 1H), 4.70-3.20 (br., 6H), 2.99/2.72 (dd+d, 2H), 2.46-2.20 (br., 2H), 1.66-1.31 (br., 6H), 1.42 (s, 9H).

HRMS-ESI (m/z) [M+H]⁺ calcd for C₂₁H₃₁N₂O₃: 359.2329, found 359.2333.

### Step B: (3S)-3-[2-(1-piperidyl)ethyl]-1,2,3,4-tetrahydroisoquinoline

Using General procedure 6c, the product of Step A (324 mg, 0.90 mmol) was deprotected, then the title product was prepared according to a modified General procedure 6a using MTBE, THF and DCM as solvents. The desired product was obtained (160 mg, 72%).

¹H NMR (500 MHz, DMSO-d6) δ ppm 7.07 (m, 1H), 7.06 (m, 1H), 7.03 (m, 1H), 6.99 (m, 1H), 3.86 (s, 2H), 2.73 (m, 1H), 2.68/2.41 (dd+dd, 2H), 2.43/2.32 (m+m, 2H), 2.33/2.29 (m+m, 4H), 1.59/1.53 (m+m, 2H), 1.48 (qn, 4H), 1.37 (m, 2H); ¹³C NMR (125 MHz, DMSO-d6) δ ppm 136.7, 135.5, 129.3, 126.3, 126, 125.8, 56.4, 54.7, 52.7, 48.3, 35.6, 33.7, 26.1, 24.7.

HRMS-ESI (m/z) [M+H]⁺ calcd for C₁₆H₂₅N₂: 245.2012, found 245.2015.

### Step C. N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-[2-(piperidin-1-yl)ethyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from Preparation IX (50 mg, 0.064 mmol) as the appropriate carboxylic acid and the product of Step B (35 mg, 1.4 eq.) as the appropriate amine, the title product was obtained (18.1 mg, 28%).

HRMS-ESI (m/z) [M+H]⁺ calcd for C₆₀H₆₈FN₈O₆: 1015.5240, found 1015.5241.

### Example 75: N-(5-cyano-1,2-dimethyl-1H-pyrrol-3-yl)-5-[2-(2-{2-fluoro-4-[2-((2S or R)-2-methylmorpholin-4-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 1)

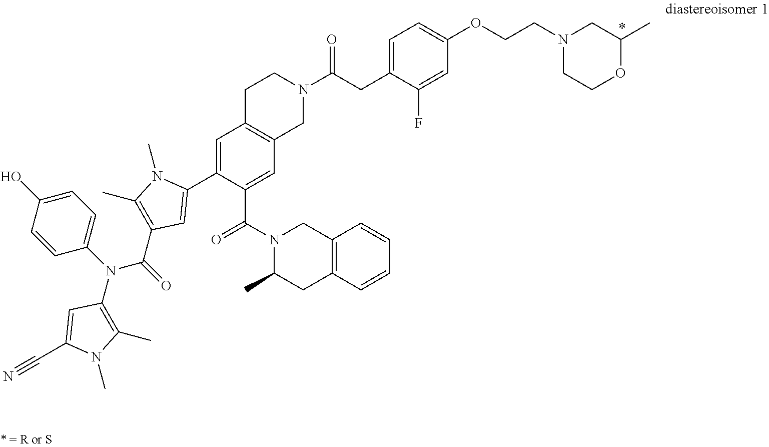 [SMILES: Cc1c(N(C(=O)c2cc(-c3cc4c(cc3C(=O)N3Cc5ccccc5C[C@H]3C)CN(C(=O)Cc3ccc(OCCN5CCOC(C)C5)cc3F)CC4)n(C)c2C)c2ccc(O)cc2)cc(C#N)n1C]

### Step A: methyl 2-[2-fluoro-4-[2-((2S)-2-methylmorpholin-4-yl)ethoxy]phenyl]acetate and methyl 2-[2-fluoro-4-[2-((2R)-2-methylmorpholin-4-yl)ethoxy]phenyl]acetate

Using the General procedure 7d, starting from Preparation X (500 mg, 1.72 mmol) as the appropriate alkyl halide and 2-methylmorpholine (380 mg, 2.2 eq.) as the appropriate amine, the desired product was obtained (510 mg, 95%).

¹H NMR (500 MHz, DMSO-d6) δ ppm 7.22 (t, 1H), 6.83 (dd, 1H), 6.75 (t, 2H), 6.75 (dd, 1H), 3.72/3.48 (dm+td, 2H), 3.63 (s, 2H), 3.61 (s, 3H), 3.49 (m, 1H), 2.81/1.78 (dm+dd, 2H), 2.74/2.08 (dm+td, 2H), 2.66 (t, 2H), 1.03 (d, 3H); ¹³C NMR (125 MHz, DMSO-d6) δ ppm 171.5, 161.5, 159.5, 132.7, 113.7, 111.1, 102.3, 71.5, 66.3, 66.1, 60.5, 57.1, 53.3, 52.3, 33.6, 19.5.

HRMS-ESI (m/z) [M+H]⁺ calcd for C₁₆H₂₃FNO₄: 312.1606, found 312.1608.

Enantiomers of the desired product was separated on an AD-3 chiral column (100*500 mm, 20 m) using 30:70 EtOH-heptane+0.1% DEA as eluents to give the Enantiomer 1 (99.9% ee) and Enantiomer 2 (99.8% ee).

### Step B: 2-[2-fluoro-4-[2-((2S or R)-2-methylmorpholin-4-yl)ethoxy]phenyl]acetic acid (from Enantiomer 1)

Using the General procedure 8, starting from the Enantiomer 1 of Step A (255 mg, 0.82 mmol), the desired product was obtained (146 mg, 60%).

¹H NMR (500 MHz, DMSO-d6) δ ppm 12.38 (br., 1H), 7.20 (t, 1H), 6.81 (dd, 1H), 6.73 (dd, 1H), 4.07 (t, 2H), 3.72/3.48 (dm+td, 2H), 3.52 (s, 2H), 3.50 (m, 1H), 2.81/1.78 (dm+dd, 2H), 2.74/2.08 (dm+td, 2H), 2.66 (t, 2H), 1.03 (d, 3H); ¹³C NMR (125 MHz, DMSO-d6) δ ppm 172.5, 161.6, 159.3, 132.7, 114.5, 111.0, 102.2, 71.5, 66.3, 66.1, 60.5, 57.1, 53.3, 34, 19.5.

HRMS-ESI (m/z) [M+H]⁺ calcd for C₁₅H₂₁FNO₄: 298.1449, found 298.1450.

### Step C: N-(5-cyano-1,2-dimethyl-1H-pyrrol-3-yl)-5-[2-(2-{2-fluoro-4-[2-((2S or R)-2-methylmorpholin-4-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 1)

Using General procedure 4c, starting from the product of Step B (140.0 mg, 4.5 eq.) as the appropriate carboxylic acid and Preparation VIIb (80 mg, 0.104 mmol) as the appropriate amine, afforded the title compound (79.0 mg, 81%).

HRMS-ESI (m/z) [M+H]⁺ calcd for C₅₅H₅₉FN₇O₆: 932.4511, found 932.4505.

### Example 76: N-(5-cyano-1,2-dimethylpyrrol-3-yl)-5-[2-(2-{4-[2-(4-fluoropiperidin-1-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

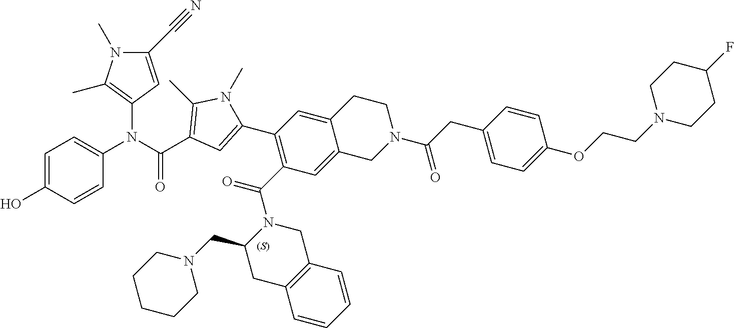

Using General procedure 4a and Preparation VIIa (150 mg, 0.18 mmol, 1 eq.) as the appropriate amine and the product of Step A in Example 53 (52 mg, 0.18 mmol, 1 eq.) as the appropriate carboxylic acid afforded an intermediate which was treated according to General procedure 3c to afford the title product (67.3 mg, 0.07 mmol, 37%).

HRMS calcd for C₆₀H₆₇FN₈O₅: 998.5218, found 999.5294 [M+H]⁺

### Example 77: N-[5-cyano-1-(difluoromethyl)-2-methylpyrrol-3-yl]-N-(4-hydroxyphenyl)-1,2-dimethyl-5-[2-(2-{4-[2-(morpholin-4-yl)ethoxyphenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]pyrrole-3-carboxamide

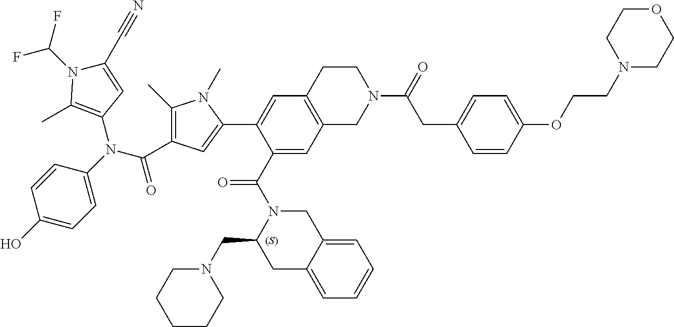

### Step A: 1-(difluoromethyl)-5-methyl-4-({4-[(triisopropylsilyl)oxy]phenyl}amino)pyrrole-2-carbonitrile

To a solution of Preparation IIIb (4.52 g, 17.02 mmol, 2 eq.) in 1,4-dioxane (85 mL) was added 4-bromo-1-(difluoromethyl)-5-methyl-1H-pyrrole-2-carbonitrile (2 g, 8.51 mmol, 1 eq.) and Cs₂CO₃ (8.32 g, 25.53 mmol, 3 eq.) and the mixture was sparged with N₂. BrettPhos Pd G3 (759 mg, 0.85 mmol, 0.1 eq.) was added and the mixture was sparged with N₂ and then stirred at 100° C. for 18 h. The mixture was allowed to cool to rt, diluted with DCM and water and then filtered through a celite cartridge washing with DCM and water. The filtrate layers were separated by the addition of brine. The layers were separated, and the aqueous phase was extracted with DCM. The combined organic extracts were dried over MgSO₄ and concentrated in vacuo. Purification by automated flash chromatography eluting with a gradient of 0-20% EtOAc in heptane afforded the title product (3.11 g, 7.4 mmol, 87%).

LRMS calcd for C₂₂H₃₁F₂N₃OSi. 419.2, found 420.4 [M+H]⁺

¹H NMR (400 MHz, DMSO-d6) δ ppm: 7.79 (t, J=57.5 Hz, 1H), 7.15 (s, 1H), 7.10 (s, 1H), 6.70-6.63 (m, 2H), 6.60-6.53 (m, 2H), 2.24 (s, 3H), 1.25-1.13 (m, 3H), 1.04 (d, J=7.3 Hz, 18H).

### Step B: tert-butyl 6-(4-{[5-cyano-1-(difluoromethyl)-2-methylpyrrol-3-yl]({4-[(triisopropylsilyl)oxy]phenyl})carbamoyl}-1,5-dimethylpyrrol-2-yl)-7-f[(3S)-3-(piperidin-1-ylmethyl)-3,4-dihydro-1H-isoquinolin-2-yl]carbonyl}-3,4-dihydro-1H-isoquinoline-2-carboxylate

Using General procedure 2a and Preparation IIb (253 mg, 0.4 mmol, 1 eq.) as the appropriate acid and the product from Step A (254 mg, 0.61 mmol, 1.5 eq.) as the appropriate aniline afforded the title product (220 mg, 0.21 mmol, 53%).

LRMS calcd for C₅₉H₇₅F₂N₇O₅Si: 1027.6, found 1028.6 [M+H]⁺

### Step C: N-[5-cyano-1-(difluoromethyl)-2-methylpyrrol-3-yl]-1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)-N-{4-[(triisopropylsilyl)oxy]phenyl}pyrrole-3-carboxamide

Using General procedure 3b and the product from Step B (220 mg, 0.21 mmol, 1 eq.) as the appropriate silyl-derivative afforded the title product (115 mg, 0.12 mmol, 58%).

LRMS calcd for C₅₄H₆₇F₂N₇O₃Si: 927.5, found 928.6 [M+H]⁺

### Step D: N-[5-cyano-1-(difluoromethyl)-2-methylpyrrol-3-yl]-1,2-dimethyl-5-[2-(2-{4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-{4-[(triisopropylsilyl)oxy]phenyl}pyrrole-3-carboxamide

Using General procedure 4a and the product from Step C (57.5 mg, 0.06 mmol, 1 eq.) as the appropriate amine and Preparation Va (18 mg, 0.07 mmol, 1.1 eq.) as the appropriate carboxylic acid afforded the title product (65 mg, 0.06 mmol, 89%).

LRMS calcd for C₆₈H₈₄F₂N₈O₆Si: 1174.6, found 1175.8 [M+H]⁺

### Step E: N-[5-cyano-1-(difluoromethyl)-2-methylpyrrol-3-yl]-N-(4-hydroxyphenyl)-1,2-dimethyl-5-[2-(2-{4-[2-(morpholin-4-yl)ethoxy]phenyl}acetyl)-7-f[(3S)-3-(piperidin-1-ylmethyl)-3,4-dihydro-1H-isoquinolin-2-yl]carbonyl}-3,4-dihydro-1H-isoquinolin-6-yl]pyrrole-3-carboxamide

Using General procedure 3c and the product from Step D (65 mg, 0.06 mmol, 1 eq.) as the appropriate silyl-derivative afforded the title product (13.3 mg, 0.01 mmol, 24%).

HRMS calcd for C₅₉H₆₄F₂N₈O₆: 1018.4917, found 1019.4988 [M+H]⁺

### Example 78: N-[5-cyano-1-(difluoromethyl)-2-methylpyrrol-3-yl]-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

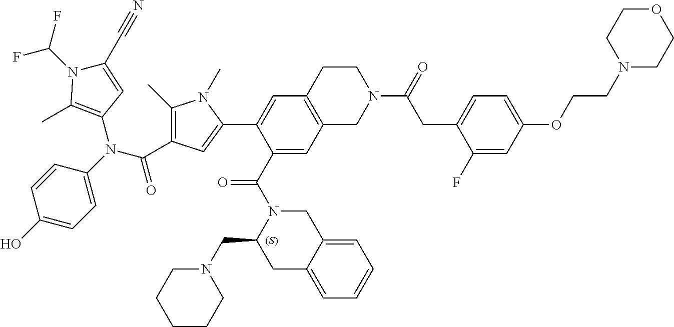

Using General procedure 4a and the product from Example 77, Step C (57.5 mg, 0.06 mmol, 1 eq.) as the appropriate amine and Preparation Vb (19 mg, 0.07 mmol, 1.1 eq.) as the appropriate carboxylic acid afforded an intermediate which was treated according to General procedure 3c to afford the title product (6.1 mg, 0.01 mmol, 10%).

HRMS calcd for C₅₉H₆₃F₃N₈O₆: 1036.4823, found 1037.4903 [M+H]⁺

### Example 79: N-(5-cyano-1,2-dimethylpyrrol-3-yl)-5-(7-{[(3S)-3-{[(3R)-3-fluoropyrrolidin-1-yl]methyl}-3,4-dihydro-1H-isoquinolin-2-yl]carbonyl}-2-(2-{4-[2-(oxan-4-yl)ethoxy]phenyl}acetyl)-3,4-dihydro-1H-isoquinolin-6-yl)-N-(4-hydroxyphenyl)-1,2-dimethylpyrrole-3-carboxamide

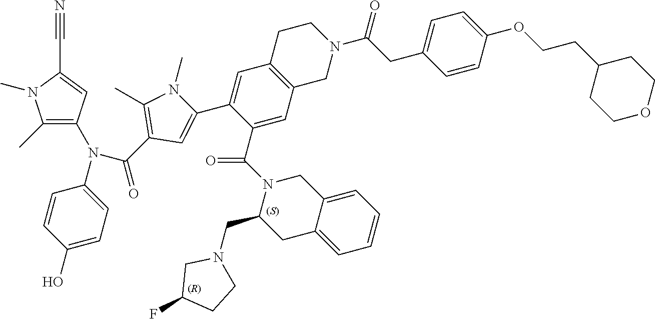

### Step A: tert-butyl (3S)-3-{[(3R)-3-fluoropyrrolidin-1-yl]carbonyl}-3,4-dihydro-1H-isoquinoline-2-carboxylate

Using General procedure 5 and Boc-(3S)-1,2,3,4-tetrahydroisoquinoline-3-carboxylic acid (3 g, 10.82 mmol, 1 eq.) and (R)-(−)-3-fluoropyrrolidine hydrochloride (1.49 g, 11.9 mmol, 1.1 eq.) as the appropriate amine afforded the title product (3.06 g, 8.78 mmol, 81%).

LRMS calcd for C₁₉H₂₅FN₂O₃: 348.2; found 249.2 [M-Boc+H]⁺

¹H NMR (400 MHz, DMSO-d6) δ ppm: 7.24-7.15 (m, 4H), 5.52-5.18 (m, 1H), 5.00-4.20 (m, 3H), 3.96-2.75 (m, 6H), 2.34-1.86 (m, 2H), 1.47-1.29 (m, 9H).

### Step B: (3S)-3-{[(3R)-3-fluoropyrrolidin-1-yl]methyl}-1,2,3,4-tetrahydroisoquinoline dihydrochloride

Using General procedure 6 and the product from Step A (2.96 g, 8.5 mmol, 1 eq.) as the appropriate THIQ amide afforded the title product (2.61 g, quant.).

LRMS calcd for C₁₄H₁₉FN₂: 234.2; found 235.2 [M+H]⁺

¹H NMR (400 MHz, DMSO-d6) δ ppm: 11.94-9.38 (br m, 3H), 7.36-7.10 (m, 4H), 5.63-5.29 (m, 1H), 4.71-3.01 (m, 11H), 2.60-1.94 (m, 2H).

### Step C. benzyl 6-[4-(ethoxycarbonyl)-1,5-dimethylpyrrol-2-yl]-7-{[(3S)-3-{[(3R)-3-fluoropyrrolidin-1-yl]methyl}-3,4-dihydro-1H-isoquinolin-2-yl]carbonyl}-3,4-dihydro-1H-isoquinoline-2-carboxylate

Using General procedure 4b and the product from Step B (2.61 g, 8.5 mmol, 1 eq.) as the appropriate amine and Preparation I (4.05 g, 8.5 mmol, 1 eq.) as the appropriate carboxylic acid, followed with a purification step by automated flash chromatography eluting with a gradient of 40-100% EtOAc in heptane afforded the title product (2.9 g, 4.19 mmol, 49%).

LRMS calcd for C₄₁H₄₅FN₄O₅: 692.3, found 693.4 [M+H]⁺

### Step D: 5-(7-{[(3S)-3-{[(3R)-3-fluoropyrrolidin-1-yl]methyl}-3,4-dihydro-1H-isoquinolin-2-yl]carbonyl}-1,2,3,4-tetrahydroisoquinolin-6-yl)-1,2-dimethylpyrrole-3-carboxylic acid

To a solution of the product from Step C (2.9 g, 4.19 mmol, 1 eq.) in MeOH (40 mL) and water (10 mL) was added 5 M aq. NaOH solution (8.37 mL, 41.86 mmol, 10 eq.) and the mixture was refluxed for 2 days. The mixture was allowed to cool to rt and MeOH removed in vacuo. Water was added (90 mL) and the mixture was neutralised and extracted with 3:1 DCM:IPA. The pH was adjusted to pH 5 and extracted again with 3:1 DCM:IPA. The combined organic extracts were dried over MgSO₄ and concentrated in vacuo. Purification by reverse phase automated flash chromatography eluting with a gradient of 10-100% MeCN in water afforded the title product (1.35 g, 2.54 mmol, 61%).

LRMS calcd for C₃₁H₃₅FN₄O₃: 530.3, found 531.4 [M+H]⁺

### Step E: 5-[2-(tert-butoxycarbonyl)-7-{[(3S)-3-{[(3R)-3-fluoropyrrolidin-1-yl]methyl}-3,4-dihydro-1H-isoquinolin-2-yl]carbonyl}-3,4-dihydro-1H-isoquinolin-6-yl]-1,2-dimethylpyrrole-3-carboxylic acid

To a solution of the product from Step D (1.29 g, 2.42 mmol, 1 eq.) in THE (30 mL) and water (3 mL) was added di-tert-butyl dicarbonate (0.54 mL, 2.54 mmol, 1.05 eq.) followed by the dropwise addition of TEA (0.67 mL, 4.84 mmol, 2 eq.). The mixture was stirred at rt for 18 h. THF was removed in vacuo and the residue partitioned between EtOAc and sat. aq. NH₄Cl solution. The phases were separated, and the organic phase was washed with brine, dried over MgSO₄ and concentrated in vacuo. Purification by automated flash chromatography eluting with a gradient of 0-10% MeOH in DCM afforded the title product (1.36 g, 2.16 mmol, 89%).

LRMS calcd for C₃₆H₄₃FN₄O₅: 630.3, found 631.6 [M+H]⁺

### Step F: tert-butyl 6-[4-({4-[(tert-butyldimethylsilyl)oxy]phenyl}(5-cyano-1,2-dimethylpyrrol-3-yl)carbamoyl)-1,5-dimethylpyrrol-2-yl]-7-{[(3S)-3-{[(3R)-3-fluoropyrrolidin-1-yl]methyl}-3,4-dihydro-1H-isoquinolin-2-yl]carbonyl}-3,4-dihydro-1H-isoquinoline-2-carboxylate

Using General procedure 2a and the product from Step E (1.36 g, 2.16 mmol, 1 eq.) as the appropriate acid and Preparation IVa, Step B (1.1 g, 3.23 mmol, 1.5 eq.) as the appropriate aniline afforded the title product (0.61 g, 0.63 mmol, 29%).

LRMS calcd for C₅₅H₆₈FN₇O₅Si: 953.5, found 954.4 [M+H]⁺

### Step G: N-(5-cyano-1,2-dimethylpyrrol-3-yl)-5-(7-{[(3S)-3-{[(3R)-3-fluoropyrrolidin-1-yl]methyl}-3,4-dihydro-1H-isoquinolin-2-yl]carbonyl}-1,2,3,4-tetrahydroisoquinolin-6-yl)-N-(4-hydroxyphenyl)-1,2-dimethylpyrrole-3-carboxamide dihydrochloride

Using General procedure 3b and the product from Step F (605 mg, 0.63 mmol, 1 eq.) as the appropriate silyl-derivative afforded the title product (520 mg, 100%).

LRMS calcd for C₄₄H₄₆FN₇O₃: 739.4, found 740.3 [M+H]⁺

### Step H. N-(5-cyano-1,2-dimethylpyrrol-3-yl)-5-(7-{[(3S)-3-{[(3R)-3-fluoropyrrolidin-1-yl]methyl}-3,4-dihydro-1H-isoquinolin-2-yl]carbonyl}-2-(2-{4-[2-(oxan-4-yl)ethoxy]phenyl}acetyl)-3,4-dihydro-1H-isoquinolin-6-yl)-N-(4-hydroxyphenyl)-1,2-dimethylpyrrole-3-carboxamide

Using General procedure 4a and the product from Step G (161 mg, 0.2 mmol, 1 eq.) as the appropriate amine and Preparation Vc (63 mg, 0.24 mmol, 1.2 eq.) as the appropriate carboxylic acid afforded the title product (39 mg, 0.04 mmol, 20%).

HRMS calcd for C₅₉H₆₄FN₇O₆: 985.4902, found 986.4980 [M+H]⁺

### Example 80: N-(5-cyano-1,2-dimethylpyrrol-3-yl)-5-[2-(2-{2-fluoro-4-[2-(morpholin-4-yl)ethoxy]phenyl}acetyl)-7-{[(3S)-3-{[(3R)-3-fluoropyrrolidin-1-yl]methyl}-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

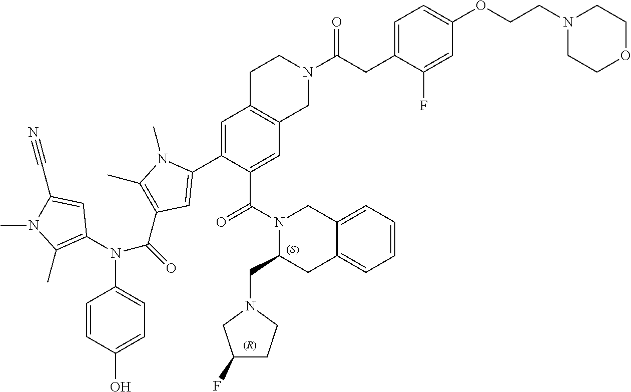

Using General procedure 4a and the product from Example 79, Step G (159 mg, 0.2 mmol, 1 eq.) as the appropriate amine and Preparation Vb (67 mg, 0.23 mmol, 1.2 eq.) as the appropriate carboxylic acid afforded the title product (43 mg, 0.04 mmol, 22%).

HRMS calcd for C₅₈H₆₂F₂N₈O₆: 1004.4760, found 1005.4836 [M+H]⁺

### Example 81: N-(3-cyano-4-methoxyphenyl)-N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxamide

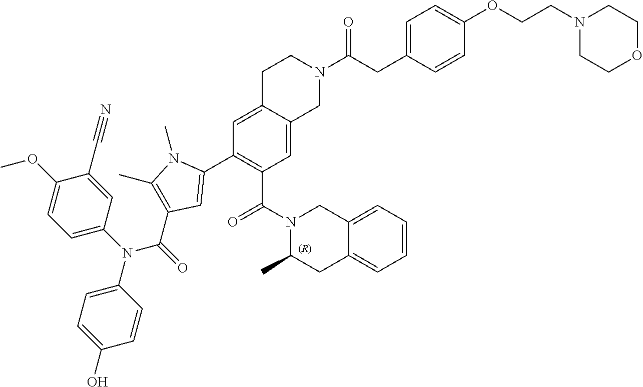

### Step A: 5-({4-[(tert-butyldimethylsilyl)oxy]phenyl}amino)-2-methoxybenzonitrile

Using General procedure 1a and 5-bromo-2-methoxybenzonitrile (162 mg, 0.76 mmol, 1 eq.) as the appropriate aryl bromide and Preparation IIa (188 mg, 0.84 mmol, 1.1 eq.) as the appropriate aniline afforded the title product (140 mg, 0.39 mmol, 52%).

LRMS calcd for C₂₀H₂₆N₂O₂Si: 354.2, found 355.0 [M+H]⁺

¹H NMR (400 MHz, DMSO-d6) δ ppm: 7.87 (s, 1H), 7.24 (dd, J=9.1, 2.9 Hz, 1H), 7.17-7.10 (m, 2H), 6.95-6.89 (m, 2H), 6.79-6.73 (m, 2H), 3.83 (s, 3H), 0.95 (s, 9H), 0.16 (s, 6H).

### Step B: N-(3-cyano-4-methoxyphenyl)-N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxamide

Using General procedure 2a and Preparation VIa (175 mg, 0.25 mmol, 1 eq.) as the appropriate acid and the product from Step A (135 mg, 0.38 mmol, 1.5 eq.) as the appropriate aniline afforded an intermediate which was treated according to General procedure 3b to afford the title product (12 mg, 0.01 mmol, 5%).

HRMS calcd for C₅₅H₅₆N₆O₇: 912.4210, found 913.4286 [M+H]⁺

### Example 82: N-(5-cyano-1,2-dimethylpyrrol-3-yl)-5-[2-(2-{4-[2-(3,3-difluoropyrrolidin-1-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

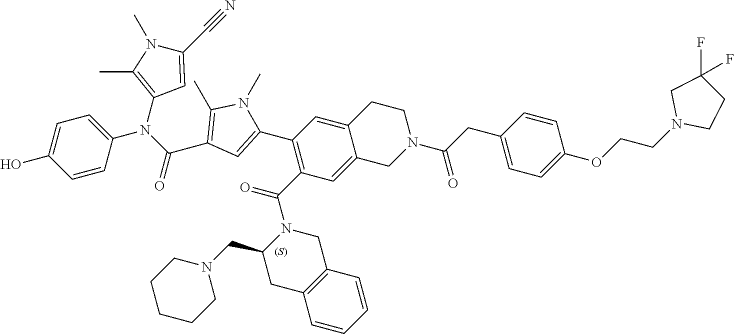

Using General procedure 4a and Preparation IVb (144 mg, 0.19 mmol, 1 eq.) as the appropriate amine and the product of Step A of Example 50 (72 mg, 0.25 mmol, 1.3 eq.) as the appropriate carboxylic acid afforded the title product (34.3 mg, 0.03 mmol, 18%).

HRMS calcd for C₅₉H₆₄F₂N₈O₅: 1002.4968, found 1003.5047 [M+H]⁺

### Example 83: N-(5-cyano-1,2-dimethylpyrrol-3-yl)-5-[2-(2-{2,5-difluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

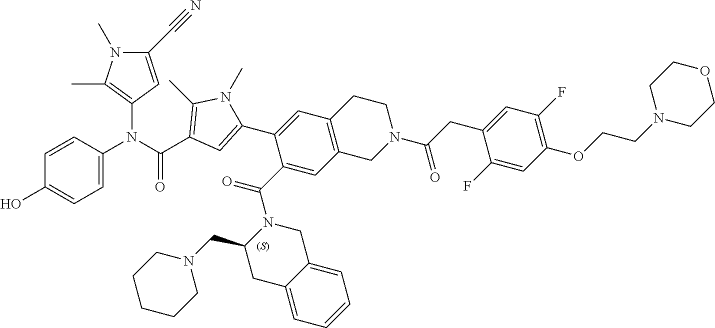

Using General procedure 4a and Preparation IVb (143.5 mg, 0.19 mmol, 1 eq) as the appropriate amine and the product from Example 69, Step D (65 mg, 0.21 mmol, 1.1 eq.) as the appropriate carboxylic acid afforded the title product (54.4 mg, 0.05 mmol, 27%).

HRMS calcd for C₅₉H₆₄F₂N₈O₆: 1018.4917, found 1019.4992 [M+H]⁺

### Example 84: N-[5-cyano-2-methyl-1-((3S or R)-tetrahydrofuran-3-ylmethyl)pyrrol-3-yl]-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereomer 2)

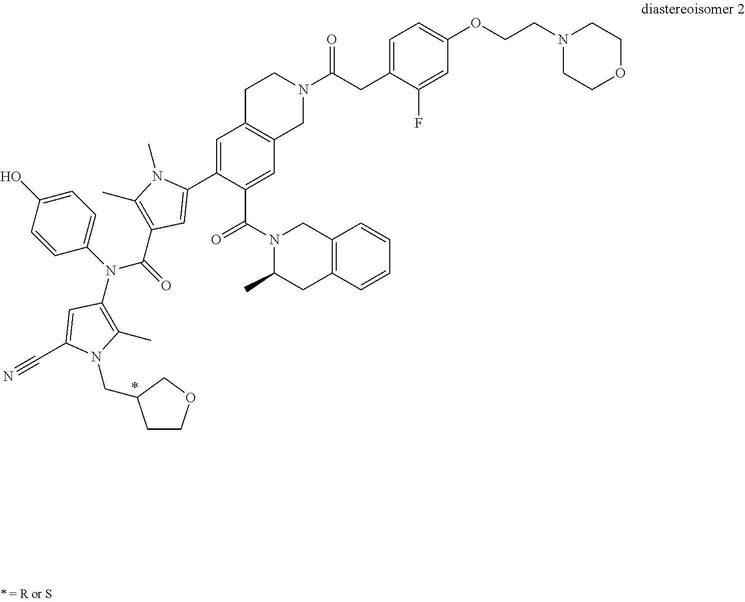 [SMILES: Cc1c(C(=O)N(c2ccc(O)cc2)c2cc(C#N)n(CC3CCOC3)c2C)cc(-c2cc3c(cc2C(=O)N2Cc4ccccc4C[C@H]2C)CN(C(=O)Cc2ccc(OCCN4CCOCC4)cc2F)CC3)n1C]

### Step A: 4-bromo-5-methyl-1-((3S)-tetrahydrofuran-3-ylmethyl)pyrrole-2-carbonitrile and 4-bromo-5-methyl-1-((3R)-tetrahydrofuran-3-ylmethyl)pyrrole-2-carbonitrile

Using the General procedure 7c, starting from the tetrahydrofuran-3-ylmethanol (1.0 mL, 2.0 eq.) as the appropriate alcohol, afforded the title compound (1.44 g, 99%).

¹H NMR (500 MHz, DMSO-d6) δ ppm 7.12 (q, 1H), 4.07/4.01 (dd+dd, 2H), 3.82/3.65 (m+m, 2H), 3.62/3.42 (dd+dd, 2H), 2.64 (m, 1H), 2.27 (d, 3H), 1.91/1.58 (m+m, 2H); ¹³C NMR (125 MHz, DMSO-d6) δ ppm 134.7, 121.0, 113.8, 103.0, 95.8, 70.0, 67.1, 49.2, 40.1, 29.3, 11.3.

HRMS-ESI (m/z) [M+NH₄]⁺ calcd for C₁₁H₁₄BrN₂O: 286.0549, found 286.0549.

Enantiomers of the desired product was separated on an AD chiral column (100*500 mm, 20 m) using 10:90 EtOH/heptane as eluents to give the Enantiomer 1 (99.9% ee) and Enantiomer 2 (99.2% ee).

### Step B: 4-[4-[tert-butyl(dimethyl)silyl]oxyanilino]-5-methyl-1-((3S or R)-tetrahydrofuran-3-ylmethyl)pyrrole-2-carbonitrile (from Enantiomer 2)

Using General procedure 1b, starting from the Enantiomer 2 of Step A (700 mg, 2.60 mmol) as the appropriate aryl-bromide and Preparation IIa (875 mg, 1.5 eq.) as the appropriate aniline, afforded the title compound (538 mg, 50%).

¹H NMR (500 MHz, DMSO-d6) δ ppm 6.89 (s, 1H), 6.78 (s, 1H), 6.61 (dm, 2H), 6.50 (dm, 2H), 4.01/3.96 (dd+dd, 2H), 3.84/3.67 (m+m, 2H), 3.63/3.44 (dd+dd, 2H), 2.66 (m, 1H), 2.12 (s, 3H), 1.93/1.61 (m+m, 2H), 0.92 (s, 9H), 0.12 (s, 6H); ¹³C NMR (125 MHz, DMSO-d6) δ ppm 146.8, 142.4, 130.4, 125.1, 120.6, 115.9, 115.1, 114.3, 99.8, 70.1, 67.1, 48.3, 40.3, 29.3, 26.1, 10.0, −4.1.

HRMS-ESI (m/z) [M+H]⁺ calcd for C₂₃H₃₄N₃O₂Si: 412.2415, found 412.2418.

### Step C. 9H-fluoren-9-ylmethyl 6-[4-[[4-[tert-butyl(dimethyl)silyl]oxyphenyl]-[5-cyano-2-methyl-1-((3S or R)-tetrahydrofuran-3-ylmethyl)pyrrol-3-yl]carbamoyl]-1,5-dimethyl-pyrrol-2-yl]-7-[(3R)-3-methyl-3,4-dihydro-1H-isoquinoline-2-carbonyl]-3,4-dihydro-1H-isoquinoline-2-carboxylate (Diastereomer 2)

Using General procedure 2b, starting from the Step B of Preparation VIIb as the appropriate acid (300 mg, 0.45 mmol) and the product of Step B (270 mg, 1.4 eq.) as the appropriate aniline, afforded the title compound (200 mg, 42%).

¹H NMR (500 MHz, DMSO-d6) δ ppm 8.05-6.21 (m, 19H), 5.57-5.00 (s, 1H), 5.36-1.29 (m, 23H), 3.48-3.06 (s, 3H), 2.45-1.86 (s, 6H), 1.12-0.47 (brd, 3H), 0.88 (s, 9H), 0.13 (s, 6H).

HRMS-ESI (m/z) [M+H]⁺ calcd for C₆₅H₇₁N₆O₆Si: 1059.5199, found 1059.5198.

### Step D: N-[5-cyano-2-methyl-1-((3S or R)-tetrahydrofuran-3-ylmethyl)pyrrol-3-yl]-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereomer 2)

To the product of Step C (120 mg, 0.11 mmol) in DMF (1 mL) was added DBU (0.003 mL, 0.2 eq.). The reaction mixture was stirred until the deprotection was completed. The mixture was treated with Preparation Vb (50 mg, 0.18 mmol) as the appropriate acid according to General procedure 4c, the title compound was obtained (45 mg, 40%).

HRMS-ESI (m/z) [M+H]⁺ calcd for C₅₈H₆₃FN₇O₇: 988.4767, found 988.4770.

### Example 85: N-(4-hydroxyphenyl)-1,2-dimethyl-5-(7-{[(3R)-3-methyl-3,4-dihydro-1H-isoquinolin-2-yl]carbonyl}-2-(2-{4-[2-(oxan-4-yl)ethoxy]phenyl}acetyl)-3,4-dihydro-1H-isoquinolin-6-yl)-N-(1-methylpyrazol-4-yl)pyrrole-3-carboxamide

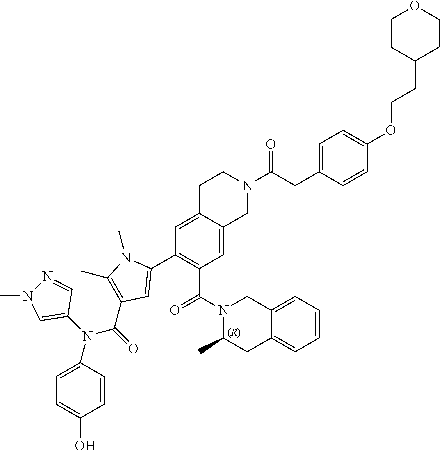

### Step A: N-{4-[(tert-butyldimethylsilyl)oxy]phenyl}-1-methylpyrazol-4-amine

Using General procedure 1a and 4-bromo-1-methyl-1H-pyrazole (0.64 mL, 6.21 mmol, 1 eq.) as the appropriate aryl bromide and Preparation IIa (1.66 g, 7.45 mmol, 1.2 eq.) as the appropriate aniline afforded the title product (1.23 g, 4.06 mmol, 65%).

LRMS calcd for C₁₆H₂₅N₃OSi: 303.2, found 304.2 [M+H]⁺

¹H NMR (400 MHz, DMSO-d6) δ ppm: 7.55 (d, J=0.9 Hz, 1H), 7.23 (d, J=0.9 Hz, 1H), 7.19 (s, 1H), 6.69-6.60 (m, 4H), 3.77 (s, 3H), 0.93 (s, 9H), 0.12 (s, 6H).

### Step B: N-(4-hydroxyphenyl)-1,2-dimethyl-5-(7-{[(3R)-3-methyl-3,4-dihydro-1H-isoquinolin-2-yl]carbonyl}-2-(2-{4-[2-(oxan-4-yl)ethoxy]phenyl}acetyl)-3,4-dihydro-1H-isoquinolin-6-yl)-N-(1-methylpyrazol-4-yl)pyrrole-3-carboxamide

Using General procedure 2a and Preparation VIb (175 mg, 0.25 mmol, 1 eq.) as the appropriate acid and the product from Step A (115 mg, 0.38 mmol, 1.5 eq.) as the appropriate aniline afforded an intermediate which was treated according to General procedure 3b to afford the title product (92 mg, 0.11 mmol, 42%).

HRMS calcd for C₅₂H₅₆N₆O₆: 860.4261, found 861.4336 [M+H]⁺

### Example 86: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-[(4-fluoropiperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

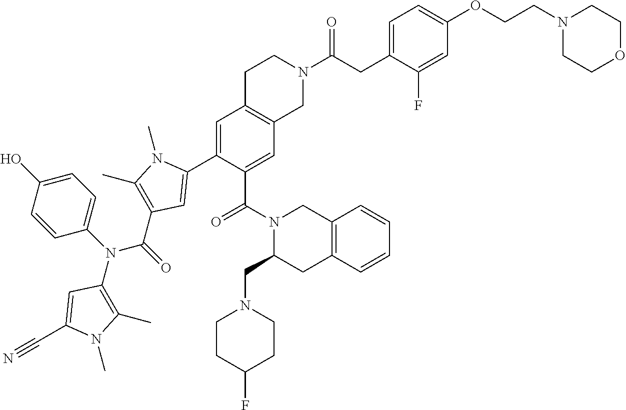 [SMILES: Cc1c(N(C(=O)c2cc(-c3cc4c(cc3C(=O)N3Cc5ccccc5C[C@H]3CN3CCC(F)CC3)CN(C(=O)Cc3ccc(OCCN5CCOCC5)cc3F)CC4)n(C)c2C)c2ccc(O)cc2)cc(C#N)n1C]

### Step A: tert-butyl (3S)-3-(4-fluoropiperidine-1-carbonyl)-3,4-dihydro-1H-isoquinoline-2-carboxylate

Using the modified General procedure 5a, starting from (3S)-2-tert-butoxycarbonyl-3,4-dihydro-]H-isoquinoline-3-carboxylic acid (800 mg, 2.885 mmol) as the appropriate carboxylic acid and 4-fluoropiperidine hydrochloride (484 mg, 1.2 eq.) as the appropriate amine in DCM (50 mL), followed with a purification step via automated flash chromatography with heptane and IPA as eluents provided the desired compound (735 mg, 70%).

¹H NMR (500 MHz, DMSO-d6) δ ppm 7.26-7.07 (m, 4H), 5.20/4.95 (dd, 1H), 4.89 (dm, 1H), 4.67/4.64/4.39/4.32 (d+d, 2H), 3.81-3.14 (m, 4H), 3.12/3.08/2.88/2.81 (dd+dd, 2H), 2.12-1.46 (m, 4H), 1.43/1.36 (brs, 9H).

HRMS-ESI (m/z) [M+H]⁺ calcd for C₂₀H₂₈FN₂O₃: 363.2078, found 363.2079.

### Step B: (3S)-3-[(4-fluoro-1-piperidyl)methyl]-1,2,3,4-tetrahydroisoquinoline

To a solution of the product of Step A (735 mg, 2.03 mmol) in ACN (20 mL) was added HCl (12M aq. solution, 1 mL, 6 eq.). The reaction mixture was stirred at rt for 1.5 h then concentrated under reduced pressure and azeotroped with toluene. The residue was dissolved in a mixture of DCM (6 mL) and THE (40 mL). This solution was treated with 1M LiAlH₄ solution in THF (4.05 mL, 2 eq.) according to General procedure 6a. Purification via flash chromatography using heptane and heptane/IPA=3/2 (0.25% TEA) as eluents afforded the desired product (345 mg, 66%).

¹H NMR (500 MHz, DMSO-d6) δ ppm 7.13-6.95 (m, 4H), 4.68 (dm, 1H), 3.90 (s, 2H), 2.92 (m, 1H), 2.65-2.22 (br., 4H), 2.65/2.37 (dd+m, 2H), 2.32 (m, 2H), 1.94-1.65 (m, 4H); ¹³C NMR (125 MHz, DMSO-d6) δ ppm 136.7, 135.2, 89.2, 63.8, 51, 50.3, 48.4, 34.1, 31.8.

HRMS-ESI (m/z) [M+H]⁺ calcd for C₁₅H₂₂FN₂: 249.1762, found 249.1763.

### Step C. N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-[(4-fluoropiperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from Preparation IX (80 mg, 0.09 mmol.) as the appropriate carboxylic acid and the product from Step B (33 mg, 1.5 eq.) as the appropriate amine, afforded the desired compound (62.6 mg, 69%).

HRMS-ESI (m/z) [M+H]⁺ calcd for C₅₉H₆₅F₂N₈O₆: 1019.4990, found 1019.4985.

### Example 87: N-(5-cyano-1,2-dimethyl-1H-pyrrol-3-yl)-N-(4-hydroxyphenyl)-5-(2-{2-[4-(methoxymethyl)phenyl]acetyl}-7-[(3R)-3-methyl-1,2,3,4-tetrahydroisoquinoline-2-carbonyl]-1,2,3,4-tetrahydroisoquinolin-6-yl)-1,2-dimethyl-1H-pyrrole-3-carboxamide

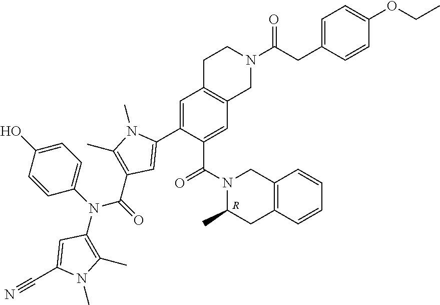

Using General procedure 4c, starting from 2-[4-(methoxymethyl)phenyl]acetic acid (53 mg, 1.5 eq.) as the appropriate carboxylic acid and Preparation VIIb (150 mg, 0.20 mmol.) as the appropriate amine, afforded the title product (110 mg, 69%).

HRMS-ESI (m/z) [M+H]⁺ calcd for C₅₀H₅₁N₆O₅: 815.3915, found 815.3913.

### Example 88: N-(5-cyano-1,2-dimethylpyrrol-3-yl)-5-{2-[2-(4-{2-[cyclopropyl(methyl)amino]ethoxy}-2-fluorophenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

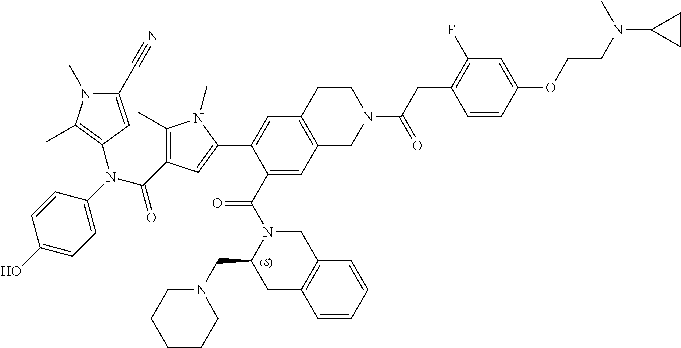

### Step A: methyl 2-(4-{2-[cyclopropyl(methyl)amino]ethoxy}-2-fluorophenyl)acetate

Using General procedure 7b and the product from Preparation Vb, Step A (270 mg, 1.47 mmol, 1 eq.) as the appropriate phenol and 2-[cyclopropyl(methyl)amino]ethan-1-ol (186 mg, 1.61 mmol, 1.1 eq.) as the appropriate alcohol afforded the title product (410.4 mg, 1.46 mmol, 100%).

LRMS calcd for C₁₅H₂₀FNO₃: 281.1, found 282.0 [M+H]⁺

¹H NMR (400 MHz, DMSO-d6) δ ppm: 7.22 (t, J=8.7 Hz, 1H), 6.80 (dd, J=12.0, 2.5 Hz, 1H), 6.76-6.71 (m, 1H), 4.07 (t, J=5.9 Hz, 2H), 3.64-3.62 (m, 2H), 3.61 (s, 3H), 2.84 (t, J=5.9 Hz, 2H), 2.33 (s, 3H), 1.77-1.69 (m, 1H), 0.45-0.39 (m, 2H), 0.32-0.27 (m, 2H).

### Step B: (4-{2-[cyclopropyl(methyl)amino]ethoxy}-2-fluorophenyl)acetic acid

To a solution of the product from Step A (410 mg, 1.46 mmol, 1 eq.) in MeOH (15 mL) was added 2 M aq. NaOH solution (1.46 mL, 2.92 mmol, 2 eq.) and the mixture was stirred at rt for 72 h. The MeOH was removed in vacuo and the aqueous residue was neutralised with 2 M aq. HCl solution and concentrated in vacuo. The residue was stirred in DCM and filtered. The filtrate was concentrated in vacuo to afford the title product which was used directly in the subsequent step without further purification (366.8 mg, 1.37 mmol, 94%).

LRMS calcd for C₁₄H₁₈FNO₃: 267.1, found 268.0 [M+H]⁺

¹H NMR (400 MHz, DMSO-d6) δ ppm: 7.18-7.10 (m, 1H), 6.69-6.60 (m, 2H), 4.03 (t, J=5.9 Hz, 2H), 3.20-3.17 (m, 2H), 2.83 (t, J=5.9 Hz, 2H), 2.32 (s, 2H), 0.45-0.38 (m, 2H), 0.32-0.27 (m, 2H).

### Step C: N-(5-cyano-1,2-dimethylpyrrol-3-yl)-5-{2-[2-(4-{2-[cyclopropyl(methyl)amino]ethoxy]-2-fluorophenyl)acetyl]-7-f[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

Using General procedure 4a and Preparation IVb (200 mg, 0.27 mmol, 1 eq.) as the appropriate amine and the product from Step B (80 mg, 0.3 mmol, 1.1 eq.) as the appropriate carboxylic acid afforded the title product (66.8 mg, 0.07 mmol, 25%).

HRMS calcd for C₅₉H₆₅FN₈O₅: 984.5062, found 985.5136 [M+H]⁺

### Example 89: N-(5-cyano-1,2-dimethylpyrrol-3-yl)-5-{2-[2-(2-fluoro-4-{[(3S)-1-(2-fluoroethyl)pyrrolidin-3-yl]oxy}phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

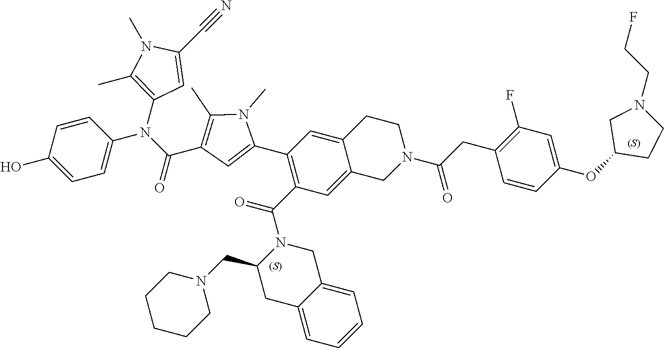

### Step A: tert-butyl (3S)-3-[3-fluoro-4-(2-methoxy-2-oxoethyl)phenoxy]pyrrolidine-1-carboxylate

Using General procedure 7b and the product from Preparation Vb, Step A (1.61 g, 8.74 mmol, 1 eq.) as the appropriate phenol and (R)-1-N-Boc-3-hydroxypyrrolidine (1.8 g, 9.62 mmol, 1.1 eq.) as the appropriate alcohol afforded the title product (3.11 g, 8.71 mmol, 100%).

LRMS calcd for C18H₂₄FNO₅: 353.2, found 254.0 [M-Boc+H]⁺

¹H NMR (400 MHz, DMSO-d6) δ ppm: 7.25 (t, J=8.7 Hz, 1H), 6.85 (dd, J=12.0, 2.5 Hz, 1H), 6.75 (dd, J=8.4, 2.5 Hz, 1H), 5.05-4.96 (m, 1H), 3.64 (d, J=1.3 Hz, 2H), 3.61 (s, 3H), 3.59-3.49 (m, 1H), 3.46-3.25 (m, 3H), 2.21-1.96 (m, 2H), 1.45-1.33 (m, 9H).

### Step B: methyl 2-{2-fluoro-4-[(3S)-pyrrolidin-3-yloxy]phenyl}acetate hydrochloride

A solution of the product from Step A (3.11 g, 8.8 mmol, 1 eq.) in 3 M HCl in MeOH (29 mL) was stirred at rt for 18 h. The solvent was removed in vacuo and then azeotroped with toluene to afford the title product (2.55 g, 8.8 mmol, 100%).

LRMS calcd for C₁₃H₁₆FNO₃: 253.1, found 254.0 [M+H]⁺

¹H NMR (400 MHz, DMSO-d6) δ ppm: 9.71-9.51 (br m, 1H), 9.51-9.30 (br m, 1H), 7.32-7.24 (m, 1H), 6.92-6.85 (m, 1H), 6.82-6.76 (m, 1H), 5.18-5.11 (m, 1H), 3.68-3.64 (m, 2H), 3.61 (s, 3H), 3.51-3.40 (m, 1H), 3.37-3.18 (m, 3H), 2.25-2.06 (m, 2H).

### Step C: (2-fluoro-4-{[(3S)-1-(2-fluoroethyl)pyrrolidin-3-yl]oxy}phenyl)acetic acid

To a solution of the product from Step B (1.28 g, 4.4 mmol, 1 eq.) in DMF (10 mL), cooled to 0° C., was added TEA (0.61 mL, 4.4 mmol, 1 eq.) followed by NaH (60% dispersion in mineral oil; 0.21 g, 5.28 mmol, 1.2 eq.) and the mixture was stirred at 0° C. for 15 mins. 1-Fluoro-2-iodoethane (0.39 mL, 4.84 mmol, 1.1 eq.) was added and the mixture was allowed to warm to rt and stirred for 18 h. The mixture was cooled to 0° C. and quenched with MeOH (15 mL). The solvent was removed in vacuo to afford an intermediate which was treated according to General procedure 8 to afford the title product (331 mg, 1.16 mmol, 71%).

LRMS calcd for C₁₄H₁₇F₂N₃: 285.1, found 286.2 [M+H]⁺

¹H NMR (400 MHz, DMSO-d6) δ ppm: 12.33 (br s, 1H), 7.21 (t, J=8.7 Hz, 1H), 6.75 (dd, J=12.0, 2.5 Hz, 1H), 6.68 (dd, J=8.5, 2.5 Hz, 1H), 4.93-4.84 (m, 1H), 4.53 (dt, J=47.7, 4.9 Hz, 2H), 3.54-3.49 (m, 2H), 2.99-2.67 (m, 5H), 2.58-2.52 (m, 1H), 2.35-2.22 (m, 1H) 1.83-1.70 (m, 1H).

### Step D: N-(5-cyano-1,2-dimethylpyrrol-3-yl)-5-{2-[2-(2-fluoro-4-{[(3S)-1-(2-fluoroethyl)pyrrolidin-3-yl]oxy}phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

Using General procedure 4a and Preparation IVb (200 mg, 0.27 mmol, 1 eq.) as the appropriate amine and the product from Step C (85 mg, 0.3 mmol, 1.1 eq.) as the appropriate carboxylic acid afforded the title product (110.7 mg, 0.11 mmol, 41%).

HRMS calcd for C₅₉H₆₄F₂N₈O₅: 1002.4968, found 1003.5041 [M+H]⁺

### Example 90: N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-(1-methylindazol-5-yl)pyrrole-3-carboxamide

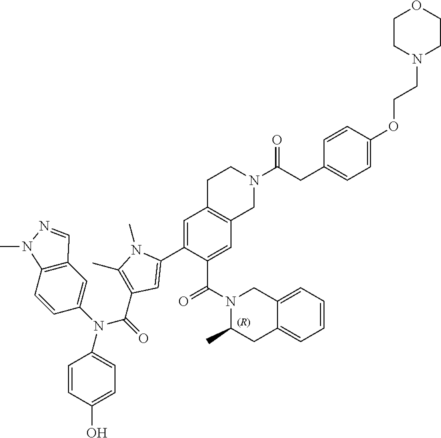

### Step A: N-{4-[(tert-butyldimethylsilyl)oxy]phenyl}-1-methylindazol-5-amine

Using General procedure 1a and 5-bromo-1-methyl-1H-indazole (400 mg, 1.9 mmol, 1 eq.) as the appropriate aryl bromide and Preparation IIIa (466 mg, 2.08 mmol, 1.1 eq.) as the appropriate aniline afforded the title product (213 mg, 0.6 mmol, 32%).

LRMS calcd for C₂₀H₂₇N₃OSi: 353.2, found 354.2 [M+H]⁺

¹H NMR (400 MHz, DMSO-d6) δ ppm: 7.82 (d, J=1.0 Hz, 1H), 7.71 (s, 1H), 7.52-7.47 (m, 1H), 7.24 (dd, J=2.1, 0.8 Hz, 1H), 7.11 (dd, J=8.9, 2.1 Hz, 1H), 6.97-6.90 (m, 2H), 6.76-6.70 (m, 2H), 3.98 (s, 3H), 0.95 (s, 9H), 0.16 (s, 6H).

### Step B: N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-(1-methylindazol-5-yl)pyrrole-3-carboxamide

Using General procedure 2a and Preparation VIa (175 mg, 0.25 mmol, 1 eq) as the appropriate acid and the product from Step A (134 mg, 0.38 mmol, 1.5 eq) as the appropriate aniline afforded an intermediate which was treated according to General procedure 3b to afford the title product (7.5 mg, 0.01 mmol, 3%).

HRMS calcd for C₅₅H₅₇N₇O₆: 911.4370, found 912.4442 [M+H]⁺

### Example 91: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[methyl(propan-2-yl)amino]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

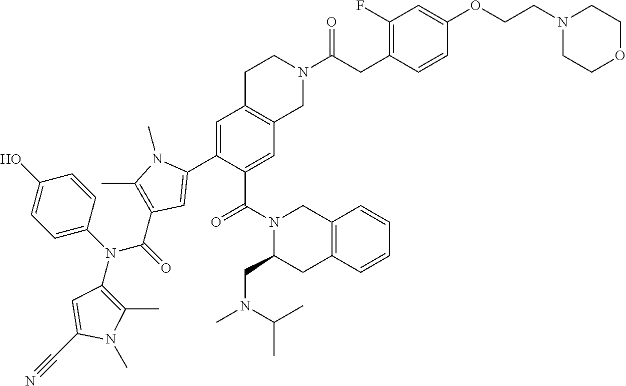 [SMILES: Cc1c(N(C(=O)c2cc(-c3cc4c(cc3C(=O)N3Cc5ccccc5C[C@H]3CN(C)C(C)C)CN(C(=O)Cc3ccc(OCCN5CCOCC5)cc3F)CC4)n(C)c2C)c2ccc(O)cc2)cc(C#N)n1C]

### Step A: tert-butyl (3S)-3-[isopropyl(methyl)carbamoyl]-3,4-dihydro-1H-isoquinoline-2-carboxylate

Using the General procedure 5a, starting from (3S)-2-tert-butoxycarbonyl-3,4-dihydro-]H-isoquinoline-3-carboxylic acid (800 mg 2.88 mmol) as the appropriate carboxylic acid and N-methylpropan-2-amine (0.360 mL, 1.2 eq.) as the appropriate amine, afforded the desired product (450 mg, 44%).

¹H NMR (500 MHz, DMSO-d6) δ ppm 7.22-7.10 (m, 4H), 5.50-4.00 (brs, 2H), 4.69/4.35 (d+d, 2H), 3.30-2.50 (brm, 5H), 1.40 (s, 9H), 1.30-0.80 (brs, 6H).

HRMS-ESI (m/z) [M+H]⁺ calcd for C₁₉H₂₉N₂O₃: 333.2173, found 333.2174.

### Step B: N-methyl-N-[[(3S)-1,2,3,4-tetrahydroisoquinolin-3-yl]methyl]propan-2-amine

To a solution of the product of Step A (450 mg, 1.35 mmol) in ACN (20 mL) was added HCl (12M aq. solution, 1.0 mL, 8.8 eq.). The reaction mixture was stirred until the deprotection was complete, then concentrated under reduced pressure and azeotroped with toluene. The residue was dissolved in a mixture of DCM (5 mL) and MTBE (7 mL). This solution was treated with 1M LiAlH₄ solution in THE (2.7 mL, 2 eq.) following General procedure 6a, affording the title compound (110 mg, 35%).

¹H NMR (500 MHz, DMSO-d6) δ ppm 7.08 (m, 1H), 7.07 (m, 1H), 7.06 (m, 1H), 7.01 (m, 1H), 3.90 (s, 2H), 2.83 (m, 1H), 2.77 (m, 1H), 2.64/2.37 (dd+dd, 2H), 2.35/2.31 (dd+dd, 2H), 2.16 (s, 3H), 0.95 (d, 6H); ¹³C NMR (125 MHz, DMSO-d6) δ ppm 129.4, 126.5, 126.1, 125.8, 58.7, 54.1, 51.6, 48.6, 37.8, 34.2, 18.3/17.9.

HRMS-ESI (m/z) [M+H]⁺ calcd for C₁₄H₂₃N₂: 219.1856, found 219.1858.

### Step C. N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[methyl(propan-2-yl)amino]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from Preparation IX (90 mg, 0.10 mmol) as the appropriate carboxylic acid and the product of Step B (44 mg, 2.0 eq.) as the appropriate amine, afforded the title compound (55 mg, 56%).

HRMS-ESI (m/z) [M+H]⁺ calcd for C₅₈H₆₆FN₈O₆: 989.5089, found 989.5086.

### Example 92: N-(5-cyano-1,2-dimethyl-pyrrol-3-yl)-5-[2-[2-[2-fluoro-4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-1,2-dimethyl-N-phenyl-pyrrole-3-carboxamide

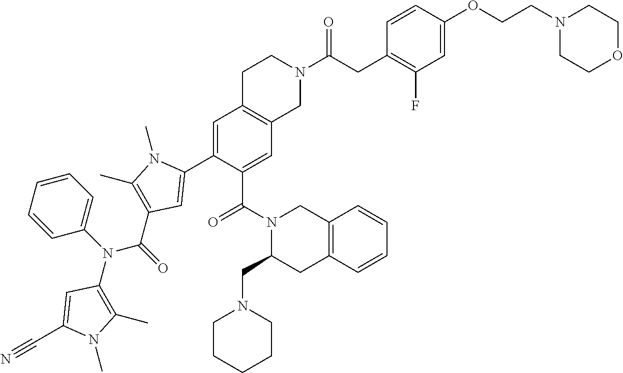 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCOCC5)cc3F)CC4)n(C)c2C)c2ccccc2)cc(C#N)n1C]

### Step A: 2-benzyloxycarbonyl-6-[4-[(5-cyano-1,2-dimethyl-pyrrol-3-yl)-phenyl-carbamoyl]-1,5-dimethyl-pyrrol-2-yl]-3,4-dihydro-1H-isoquinoline-7-carboxylic acid

Using General procedure 2b, starting from the product of Step B of Preparation IX (350 mg, 0.69 mmol) as the appropriate acid and the product of Step A of Example 65 (176 mg, 1.2 eq.) as the appropriate amine, afforded the title compound as major product (160 mg, 36%) and 02-benzyl 07-tert-butyl 6-[4-[(5-cyano-1,2-dimethyl-pyrrol-3-yl)-phenyl-carbamoyl]-1,5-dimethyl-pyrrol-2-yl]-3,4-dihydro-]H-isoquinoline-2,7-dicarboxylate (60 mg, 12%) as minor product.

¹H NMR (400 MHz, DMSO-d6) δ ppm 12.74 (brs, 1H), 7.61-6.79 (m, 13H), 5.15 (s, 1H), 5.14 (s, 2H), 4.69-2.78 (m, 6H), 3.58/3.15 (s+s, 6H), 2.39/1.98 (s+s, 6H); ¹³C NMR (100 MHz, DMSO-d6) δ ppm 108.1, 66.7, 33.2/31.6, 11.9/10.1.

HPLC-MS-ESI (m/z) [M+H]⁺ calcd for C₃₈H₃₆N₅O₅: 642.3, found 642.4.

### Step B: benzyl 6-[4-[(5-cyano-1,2-dimethyl-pyrrol-3-yl)-phenyl-carbamoyl]-1,5-dimethyl-pyrrol-2-yl]-7-[(3S)-3-(1-piperidylmethyl)-3,4-dihydro-1H-isoquinoline-2-carbonyl]-3,4-dihydro-1H-isoquinoline-2-carboxylate

Using a modified General Procedure 4c without the deprotection step, starting from the major carboxylic acid product of Step B (155 mg, 0.24 mmol) as the appropriate acid and the product of Step B of Preparation IIb (109 mg, 1.5 eq.) as the appropriate amine, afforded the title compound (123 mg, 60%).

¹H NMR (400 MHz, DMSO-d6) δ ppm 7.47-6.82 (m, 16H), 6.78-6.48 (s, 1H), 5.51-5.20 (s, 1H), 5.20-5.01 (s, 2H), 5.10-1.60 (m, 17H), 3.65-3.03 (s, 6H), 2.44-1.79 (s, 6H), 1.58-1.12 (m, 6H).

HPLC-ESI-MS (m/z) [M+H]⁺ calcd for C₅₃H₅₆N₇O₄: 854.4, found 854.4.

### Step C: N-(5-cyano-1,2-dimethyl-pyrrol-3-yl)-1,2-dimethyl-N-phenyl-5-[7-[(3S)-3-(1-piperidylmethyl)-3,4-dihydro-1H-isoquinoline-2-carbonyl]-1,2,3,4-tetrahydroisoquinolin-6-yl]pyrrole-3-carboxamide

Using General procedure 6b, starting from the product of Step C (112 mg. 0.13 mmol) afforded the title product (66 mg, 66%).

¹H NMR (400 MHz, DMSO-d6) δ ppm 7.24-6.49 (m, 12H), 5.50-5.20 (s, 1H), 5.06-1.22 (m, 23H), 3.60-3.06 (s, 6H), 2.41-1.82 (s, 6H).

HPLC-MS-ESI (m/z) [M+H]⁺ calcd for C₄₅H₅₀N₇O₂: 720.4, found 720.4.

### Step D: N-(5-cyano-1,2-dimethyl-pyrrol-3-yl)-5-[2-[2-[2-fluoro-4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-1,2-dimethyl-N-phenyl-pyrrole-3-carboxamide

Using a modified General Procedure 4c without the deprotection step, starting from Preparation Vb as the appropriate acid (42 mg, 2 eq.) and the product of Step C (60 mg, 0.083 mmol) as the appropriate amine, the desired product was obtained (22.4 mg, 27%).

HRMS-ESI (m/z) [M+H]⁺ calcd for C₅₉H₆₆FN₈O₅: 985.5135, found 985.5137.

### Example 93: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-{2-[4-(2-{4-oxa-7-azaspiro[2.5]octan-7-yl}ethoxy)phenyl]acetyl}-1,2,3,4-tetrahydroisoquinolin-6-yl}-1H-pyrrole-3-carboxamide

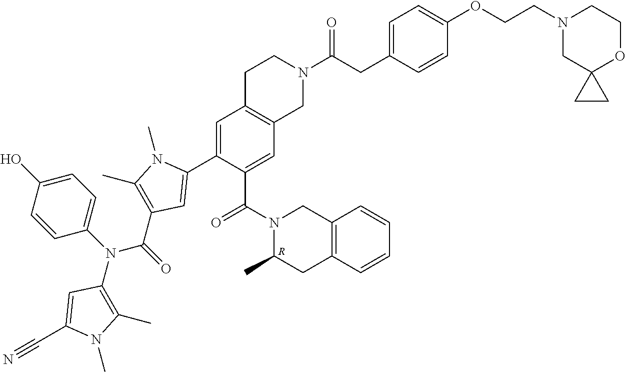

### Step A: 2-[4-[2-(4-oxa-7-azaspiro[2.5]octan-7-yl)ethoxy]phenyl]acetic acid

Using General procedure 7d, then General procedure 8, starting from methyl 2-[4-(2-bromoethoxy)phenyl]acetate (300 mg, 1.1 mmol) and 4-oxa-7-azaspiro[2.5]octane hydrochloride (197 mg, 1.2 eq.) as reactants, afforded the title product (186 mg, 58%).

¹H NMR (500 MHz, DMSO-d6) δ ppm 12.27/11.35 (brs, 2H), 7.18 (m, 2H), 6.91 (m, 2H), 4.8-2.1 (brm, 10H), 3.49 (s, 2H), 1.00-0.30 (brm, 4H).

HRMS-ESI (m/z) [M+H]⁺ calcd for C₁₆H₂₂NO₄: 292.1543, found 292.1544.

### Step B: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-{2-[4-(2-{4-oxa-7-azaspiro[2.5]octan-7-yl}ethoxy)phenyl]acetyl}-1,2,3,4-tetrahydroisoquinolin-6-yl}-1H-pyrrole-3-carboxamide

Using General procedure 4c, starting from the product of Step A (85 mg, 1.5 eq.) as the appropriate carboxylic acid and Preparation VIIb (150 mg, 0.20 mmol) as the appropriate amine, afforded the title compound (47 mg, 26%).

HRMS-ESI (m/z) [M+H]⁺ calcd for C₅₆H₆₀N₇O₆: 926.4600, found 926.4603.

### Example 94: N-[5-cyano-1-(difluoromethyl)-2-methylpyrrol-3-yl]-5-[2-(2-{2-fluoro-4-[2-(morpholin-4-yl)ethoxy]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

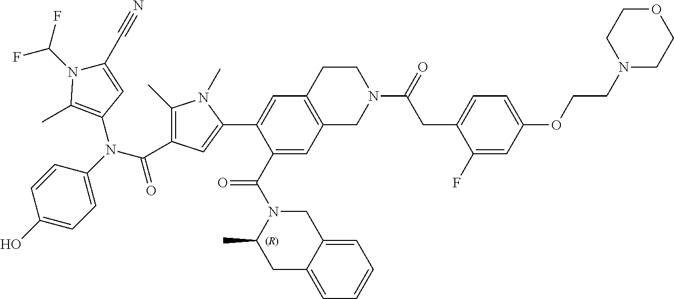

### Step A: tert-butyl 6-(4-{[5-cyano-1-(difluoromethyl)-2-methylpyrrol-3-yl]({4-[(triisopropylsilyl)oxy]phenyl})carbamoyl}-1,5-dimethylpyrrol-2-yl)-7-{[(3R)-3-methyl-3,4-dihydro-1H-isoquinolin-2-yl]carbonyl}-3,4-dihydro-1H-isoquinoline-2-carboxylate

Using General procedure 2a and Preparation IIa (1.19 g, 2.19 mmol, 1 eq.) as the appropriate acid and the product from Example 77, Step A (1.38 g, 3.29 mmol, 1.5 eq.) as the appropriate aniline afforded the title product (1.19 g, 1.26 mmol, 57%).

LRMS calcd for C₅₄H₆₆F₂N₆O₅Si: 944.5, found 945.6 [M+H]⁺

### Step B: N-[5-cyano-1-(difluoromethyl)-2-methylpyrrol-3-yl]-1,2-dimethyl-5-(7-{[(3R)-3-methyl-3,4-dihydro-1H-isoquinolin-2-yl]carbonyl}-1,2,3,4-tetrahydroisoquinolin-6-yl)-N-{4-[(triisopropylsilyl)oxy]phenyl}pyrrole-3-carboxamide

Using General procedure 3b and the product from Step A (1.19 g, 1.26 mmol, 1 eq.) as the appropriate silyl-derivative afforded the title product (700 mg, 0.83 mmol, 66%).

LRMS calcd for C₄₉H₅₈F₂N₆O₃Si: 844.4, found 845.6 [M+H]⁺

### Step C: N-[5-cyano-1-(difluoromethyl)-2-methylpyrrol-3-yl]-5-[2-(2-{2-fluoro-4-[2-(morpholin-4-yl)ethoxy]phenyl}acetyl)-7-{[(3R)-3-methyl-3,4-dihydro-1H-isoquinolin-2-yl]carbonyl}-3,4-dihydro-1H-isoquinolin-6-yl]-1,2-dimethyl-N-{4-[(triisopropylsilyl)oxy]phenyl}pyrrole-3-carboxamide

Using General procedure 4a and the product from Step B (233 mg, 0.28 mmol, 1 eq.) as the appropriate amine and Preparation Vb (87 mg, 0.308 mmol, 1.1 eq.) as the appropriate carboxylic acid afforded the title product (309 mg, 0.28 mmol, 99%).

LRMS calcd for C₆₃H₇₄F₃N₇O₆Si: 1109.5, found 1110.6 [M+H]⁺

### Step D: N-[5-cyano-1-(difluoromethyl)-2-methylpyrrol-3-yl]-5-[2-(2-{2-fluoro-4-[2-(morpholin-4-yl)ethoxy]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 3b and the product from Step C (311 mg, 0.28 mmol, 1 eq.) as the appropriate silyl-derivative, and heating at 50° C., afforded the title product (86.4 mg, 0.09 mmol, 32%).

HRMS calcd for C₅₄H₅₄F₃N₇O₆: 953.4088, found 954.4163 [M+H]⁺

### Example 95: N-[5-cyano-1-(difluoromethyl)-2-methylpyrrol-3-yl]-N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxamide

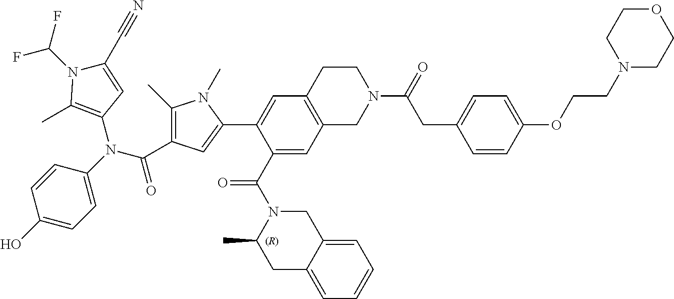

Using General procedure 4a and the product from Example 94, Step B (233 mg, 0.28 mmol, 1 eq.) as the appropriate amine and Preparation Va (82 mg, 0.308 mmol, 1.1 eq.) as the appropriate carboxylic acid afforded an intermediate. This latter was treated according to General procedure 3b and heated at 50° C., to afford the title product (57.8 mg, 0.06 mmol, 22%).

HRMS calcd for C₅₄H₅₅F₂N₇O₆: 935.4182, found 936.4259 [M+H]⁺

### Example 96: 5-{7-[(3S)-3-[(azepan-1-yl)methyl]-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

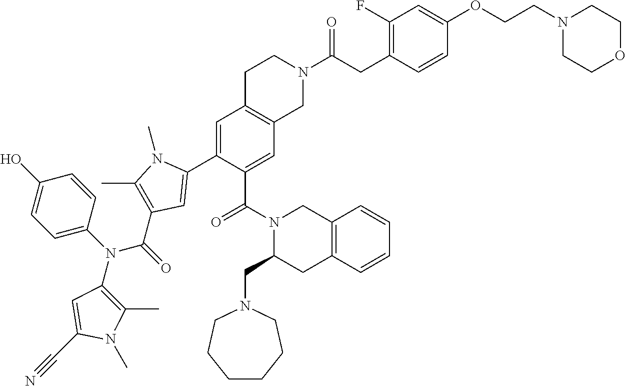 [SMILES: Cc1c(N(C(=O)c2cc(-c3cc4c(cc3C(=O)N3Cc5ccccc5C[C@H]3CN3CCCCCC3)CN(C(=O)Cc3ccc(OCCN5CCOCC5)cc3F)CC4)n(C)c2C)c2ccc(O)cc2)cc(C#N)n1C]

### Step A: tert-butyl (3S)-3-(azepane-1-carbonyl)-3,4-dihydro-1H-isoquinoline-2-carboxylate

Using the General procedure 5a, starting from (3S)-2-tert-butoxycarbonyl-3,4-dihydro-]H-isoquinoline-3-carboxylic acid (400 mg, 1.44 mmol) as the appropriate carboxylic acid and azepane (286 mg, 2.0 eq.) as the appropriate amine, afforded the desired product (238 mg, 46%).

¹H NMR (500 MHz, DMSO-d6) δ ppm 7.25-7.08 (m, 4H), 5.10/4.89 (t, 1H), 4.68/4.68/4.40/4.31 (d+d, 2H), 3.74-3.07 (m, 4H), 3.11/2.87 (dd+dd, 2H), 1.86-1.36 (m, 8H), 1.43/1.37 (s, 9H); ¹³C NMR (125 MHz, DMSO-d6) δ ppm 51.7/50.3, 51.7/50.3, 31.1, 28.6.

HRMS-ESI (m/z) [M+H]⁺ calcd for C₂₁H₃₁N₂O₃: 359.2329, found 359.2329.

### Step B: (3S)-3-(azepan-1-ylmethyl)-1,2,3,4-tetrahydroisoquinoline hydrochloride

To a solution of the product of Step A (250 mg, 0.70 mmol) in ACN (7 mL) was added HCl (12M aq. solution, 1.0 mL, 8.8 eq.). The reaction mixture was stirred until the deprotection was complete, then concentrated. The mixture was diluted with toluene and concentrated. The residue was dissolved in DCM (5 mL) and treated with 1M solution of LiAlH₄ in THF (1.4 mL, 2 eq.) followed by the General procedure 6a, to give the title compound (150 mg, 83%).

¹H NMR (400 MHz, DMSO-d6) δ ppm 7.20-6.90 (m, 4H), 3.93/3.88 (d+d, 2H), 2.83 (m, 1H), 2.70-2.54 (m, 4H), 2.65/2.36 (dd+dd, 2H), 2.52/2.42 (dd+dd, 2H), 1.66-1.48 (br., 8H); ¹³C NMR (100 MHz, DMSO-d6) δ ppm 63.4, 55.9, 52.0, 48.5, 34.1, 28.5/27.1.

HRMS-ESI (m/z) [M+H]⁺ calcd for C₁₆H₂₅N₂: 245.2011, found 245.2012.

### Step C: 5-{7-[(3S)-3-[(azepan-1-yl)methyl]-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

Using General procedure 4c, starting from Preparation IX (90 mg, 0.10 mmol) as the appropriate carboxylic acid and the product of Step B (26 mg, 1.0 eq.) as the appropriate amine, afforded the title compound (47 mg, 46%).

HRMS-ESI (m/z) [M+H]⁺ calcd for C₆₀H₆₈FN₈O₆: 1015.5246, found 1015.5241.

### Example 97: N-(5-cyano-1,2-dimethylpyrrol-3-yl)-5-[2-(2-{2,6-difluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

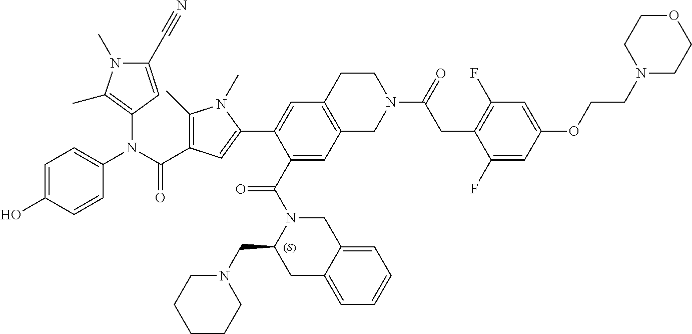

### Step A: (2,6-difluoro-4-hydroxyphenyl)acetic acid

A solution of 1 M BBr₃ in DCM (7.42 mL, 7.42 mmol, 1.5 eq) was added dropwise over 20 mins to a suspension of 2,6-difluoro-4-methoxyphenylacetic acid (1 g, 4.95 mmol, 1 eq.) in anhydrous DCM (20 mL), cooled to −78° C. under N₂. The mixture was allowed to warm to rt and stirred for 2.5 h. 30 g of crushed ice was added and the mixture was stirred vigorously for 10 mins. The phases were separated, and the aqueous phase was extracted with EtOAc. The combined organic extracts were washed with water, dried over MgSO₄ and concentrated in vacuo. Purification by reverse phase automated flash chromatography at pH 4 eluting with a gradient of 10-100% MeCN in water afforded the title product (477 mg, 2.54 mmol, 51%).

LRMS calcd for C₈H₆F₂O₃: 188.0, found 187.0 [M−H]⁻

¹H NMR (400 MHz, DMSO-d6) δ ppm: 12.51 (br s, 1H), 10.28 (br s, 1H), 6.49-6.40 (m, 2H), 3.50-3.43 (m, 2H).

### Step B: methyl 2-(2,6-difluoro-4-hydroxyphenyl)acetate

Concentrated H₂SO₄ (30 μL) was added to a solution of the product from Step A (474 mg, 2.52 mmol, 1 eq.) in MeOH (20 mL) and the mixture was refluxed for 18 h. The mixture was allowed to cool to rt, and the solvent was removed in vacuo. The residue was dissolved in EtOAc and washed with water, dried over MgSO₄ and concentrated in vacuo to afford the title product (493 mg, 2.44 mmol, 97%).

LRMS calcd for C₉H₈F₂O₃: 202.0, found 201.0 [M−H]⁻

¹H NMR (400 MHz, DMSO-d6) δ ppm: 10.34 (s, 1H), 6.51-6.41 (m, 2H), 3.62 (s, 3H), 3.58 (d, J=1.3 Hz, 2H).

### Step C: methyl 2-{2,6-difluoro-4-[2-(morpholin-4-yl)ethoxy]phenyl]acetate

Using General procedure 7a and the product from Step B (493 mg, 2.44 mmol, 1 eq.) as the appropriate phenol and 4-(2-chloroethyl)morpholine hydrochloride (908 mg, 4.88 mmol, 2 eq.) as the appropriate alkyl halide, followed with a purification step by reverse phase automated flash chromatography at pH 4 eluting with a gradient of 10-100% MeCN in water afforded the title product (540 mg, 1.71 mmol, 70%).

LRMS calcd for C₁₅H₁₉F₂NO₄: 315.1, found 316.2 [M+H]⁺

¹H NMR (400 MHz, DMSO-d6) δ ppm: 6.81-6.72 (m, 2H), 4.11 (t, J=5.7 Hz, 2H), 3.65-3.61 (m, 5H), 3.60-3.53 (m, 4H), 2.67 (t, J=5.7 Hz, 2H), 2.49-2.42 (m, 4H).

### Step D: 2-{2,6-difluoro-4-[2-(morpholin-4-yl)ethoxy]phenyl}acetic acid

Using General procedure 8 and the product from Step C (538 mg, 1.71 mmol, 1 eq.) as the appropriate ester afforded the title product (315 mg, 1.05 mmol, 61%).

LRMS calcd for C₁₄H₁₇F₂NO₄: 301.1, found 302.2 [M+H]⁺

¹H NMR (400 MHz, DMSO-d6) δ ppm: 6.74-6.63 (m, 2H), 4.08 (t, J=5.7 Hz, 2H), 3.60-3.53 (m, 4H), 3.42-3.34 (m, 2H), 2.66 (t, J=5.7 Hz, 2H), 2.49-2.40 (m, 4H).

### Step E: N-(5-cyano-1,2-dimethylpyrrol-3-yl)-5-[2-(2-{2,6-difluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and Preparation IVb (128 mg, 0.17 mmol, 1 eq.) as the appropriate amine and the product from Step D (58 mg, 0.19 mmol, 1.1 eq.) as the appropriate carboxylic acid afforded the title product (22.2 mg, 0.02 mmol, 13%).

HRMS calcd for C₅₉H₆₄F₂N₈O₆: 1018.4917, found 1019.4995 [M+H]⁺

### Example 98: N-(5-cyano-1,2-dimethylpyrrol-3-yl)-5-{2-[2-(4-{2-[cis-2,6-dimethylmorpholin-4-yl]ethoxy}phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

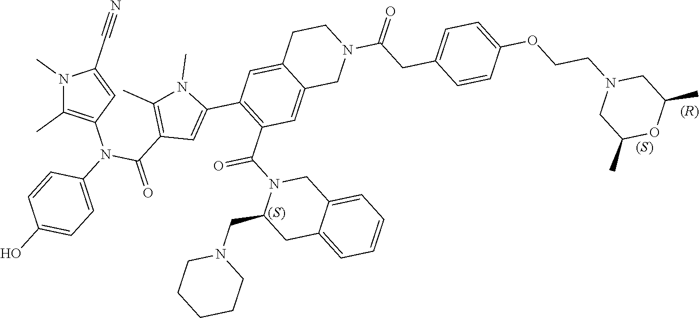

Using General procedure 4a and Preparation IVb (128 mg, 0.17 mmol, 1 eq) as the appropriate amine and the product from Example 39, Step B (56 mg, 0.19 mmol, 1.1 eq.) as the appropriate carboxylic acid afforded the title product (42 mg, 0.04 mmol, 24%).

HRMS calcd for C₆₁H₇₀N₈O₆: 1010.5418, found 1011.5491 [M+H]⁺

### Example 99: N-(5-cyano-1,2-dimethylpyrrol-3-yl)-N-(4-hydroxyphenyl)-1,2-dimethyl-5-[2-(2-{4-[2-(oxan-4-yl)ethoxy]phenyl}acetyl)-7-{[(3S)-3-(pyrrolidin-1-ylmethyl)-3,4-dihydro-1H-isoquinolin-2-yl]carbonyl}-3,4-dihydro-1H-isoquinolin-6-yl]pyrrole-3-carboxamide

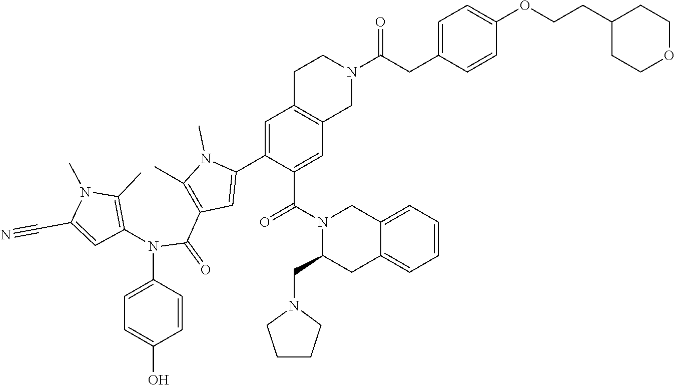 [SMILES: Cc1c(N(C(=O)c2cc(-c3cc4c(cc3C(=O)N3Cc5ccccc5C[C@H]3CN3CCCC3)CN(C(=O)Cc3ccc(OCCC5CCOCC5)cc3)CC4)n(C)c2C)c2ccc(O)cc2)cc(C#N)n1C]

Using General procedure 4a and Preparation IVc (209 mg, 0.29 mmol, 1 eq.) as the appropriate amine and Preparation Vc (84 mg, 0.319 mmol, 1.1 eq.) as the appropriate carboxylic acid afforded the title product (122.1 mg, 0.126 mmol, 43%).

HRMS calcd for C₅₉H₆₅N₇O₆: 967.4996, found 968.5074 [M+H]⁺

### Example 100: N-(5-cyano-1,2-dimethylpyrrol-3-yl)-5-[2-(2-{2-fluoro-4-[2-(morpholin-4-yl)ethoxy]phenyl}acetyl)-7-{[(3S)-3-(pyrrolidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

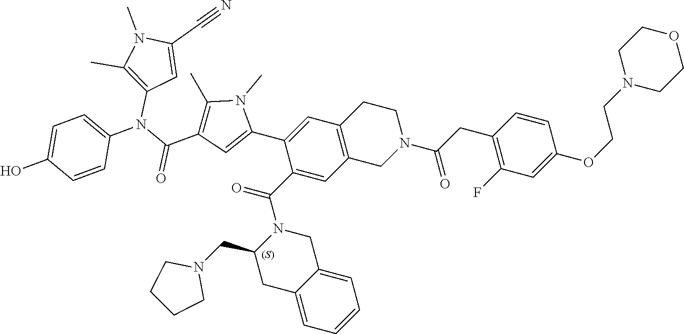

Using General procedure 4a and Preparation IVc (209 mg, 0.29 mmol, 1 eq.) as the appropriate amine and Preparation Vb (90 mg, 0.319 mmol, 1.1 eq.) as the appropriate carboxylic acid afforded the title product (113 mg, 0.114 mmol, 39%).

HRMS calcd for C₅₈H₆₃FN₈O₆: 986.4855, found 987.4926 [M+H]⁺

### Example 101: N-(5-cyano-1,2-dimethylpyrrol-3-yl)-5-{2-[2-(4-{2-[cyclopropyl(methyl)amino]ethoxy}-2-fluorophenyl)acetyl]-7-{[(3S)-3-(pyrrolidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

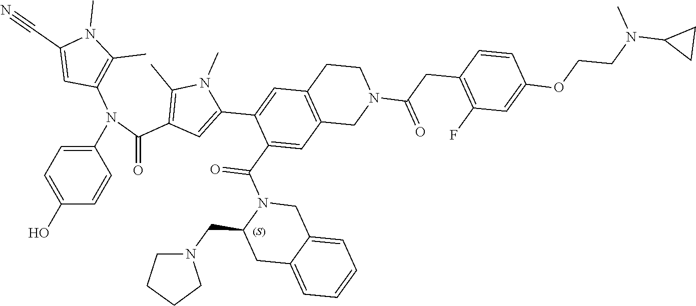

Using General procedure 4a and Preparation IVc (209 mg, 0.29 mmol, 1 eq.) as the appropriate amine and the product from Example 88, Step B (85 mg, 0.319 mmol, 1.1 eq.) as the appropriate carboxylic acid afforded the title product (141.5 mg, 0.146 mmol, 50%).

HRMS calcd for C₅₈H₆₃FN₈O₅: 970.4905, found 971.4978 [M+H]⁺

### Example 102: N-(5-cyano-1,2-dimethyl-1H-pyrrol-3-yl)-5-{2-[2-(4-{2-[cyclopropyl(methyl)amino]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

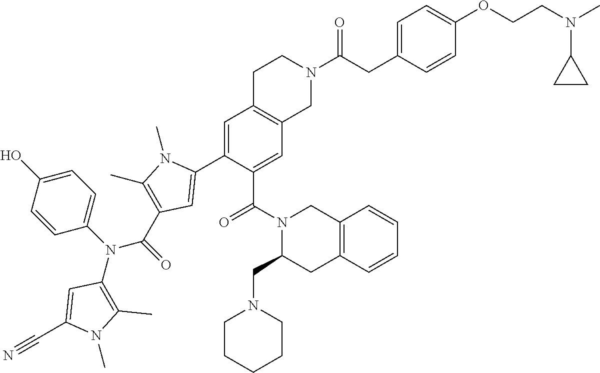 [SMILES: Cc1c(N(C(=O)c2cc(-c3cc4c(cc3C(=O)N3Cc5ccccc5C[C@H]3CN3CCCCC3)CN(C(=O)Cc3ccc(OCCN(C)C5CC5)cc3)CC4)n(C)c2C)c2ccc(O)cc2)cc(C#N)n1C]

Using General procedure 4c, starting from the product of Step G of Example 35 (59 mg, 2.5 eq.) as the appropriate carboxylic acid and Preparation VIIa (80 mg, 0.09 mmol) as the appropriate amine, afforded the title compound (64 mg, 70%).

HRMS-ESI (m/z) [M+H]⁺ calcd for C₅₉H₆₇N₈O₅: 967.5229, found 967.5227.

### Example 103: N-(5-cyano-1,2-dimethyl-1H-pyrrol-3-yl)-5-(2-{2-[2-fluoro-4-(2-{methyl[(oxetan-3-yl)methyl]amino}ethoxy)phenyl]acetyl}-7-[(3S)-3-[(piperidin-1-yl)methyl]-1,2,3,4-tetrahydroisoquinoline-2-carbonyl]-1,2,3,4-tetrahydroisoquinolin-6-yl)-N-(4-hydroxyphenyl)-1,2-dimethyl-1H-pyrrole-3-carboxamide

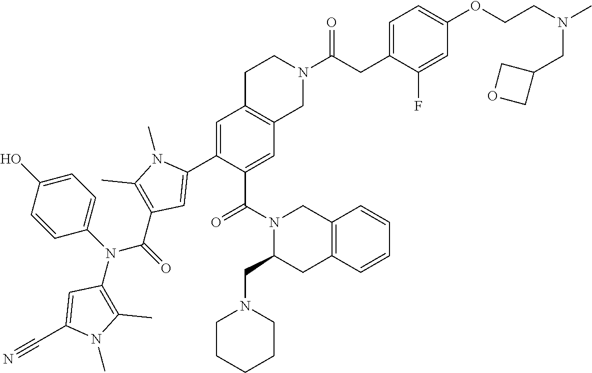 [SMILES: Cc1c(N(C(=O)c2cc(-c3cc4c(cc3C(=O)N3Cc5ccccc5C[C@H]3CN3CCCCC3)CN(C(=O)Cc3ccc(OCCN(C)CC5COC5)cc3F)CC4)n(C)c2C)c2ccc(O)cc2)cc(C#N)n1C]

### Step A: 2-[2-fluoro-4-[2-[methyl(oxetan-3-ylmethyl)amino]ethoxy]phenyl]acetic acid

Using the General procedure 7d and then General procedure 8, starting from Preparation X (200 mg, 0.69 mmol) as the appropriate alkyl halide and N-methyl-1-(oxetan-3-yl)methanamine (140 mg, 2.0 eq.) as the appropriate amine, the desired product was obtained (176 mg, 96%).

¹H NMR (500 MHz, DMSO-d6) δ ppm 11.90 (brs, 1H), 7.19 (t, 1H), 6.79 (dd, 1H), 6.71 (dd, 1H), 4.61/4.23 (dd+t, 4H), 4.02 (t, 2H), 3.48 (s, 2H), 3.14 (m, 1H), 2.70 (d, 2H), 2.67 (t, 2H), 2.17 (s, 3H); ¹³C NMR (125 MHz, DMSO-d6) δ ppm 172.7, 161.5, 159.1, 132.6, 114.9, 110.9, 102.2, 75.5, 66.7, 61.1, 56.2, 42.9, 34.4, 33.2.

HRMS-ESI (m/z) [M+H]⁺ calcd for C₁₅H₂₁FNO₄: 298.1449, found 298.1455.

### Step B: N-(5-cyano-1,2-dimethyl-1H-pyrrol-3-yl)-5-(2-{2-[2-fluoro-4-(2-{methyl[(oxetan-3-yl)methyl]amino]ethoxy)phenyl]acetyl}-7-[(3S)-3-[(piperidin-1-yl)methyl]-1,2,3,4-tetrahydroisoquinoline-2-carbonyl]-1,2,3,4-tetrahydroisoquinolin-6-yl)-N-(4-hydroxyphenyl)-1,2-dimethyl-1H-pyrrole-3-carboxamide

Using General procedure 4c, starting from the product of Step A (87 mg, 2.5 eq.) as the appropriate carboxylic acid and Preparation VIIa (100 mg, 0.12 mmol) as the appropriate amine, afforded the title compound (79 mg, 69%).

HRMS-ESI (m/z) [M+H]⁺ calcd for C₆₀H₆₈FN₈O₆: 1015.5240, found 1015.5251.

### Example 104: N-(5-cyano-1,2-dimethyl-1H-pyrrol-3-yl)-N-(4-hydroxyphenyl)-1,2-dimethyl-5-(2-{2-[4-(2-{methyl[(oxetan-3-yl)methyl]amino}ethoxy)phenyl]acetyl}-7-[(3S)-3-[(piperidin-1-yl)methyl]-1,2,3,4-tetrahydroisoquinoline-2-carbonyl]-1,2,3,4-tetrahydroisoquinolin-6-yl)-1H-pyrrole-3-carboxamide

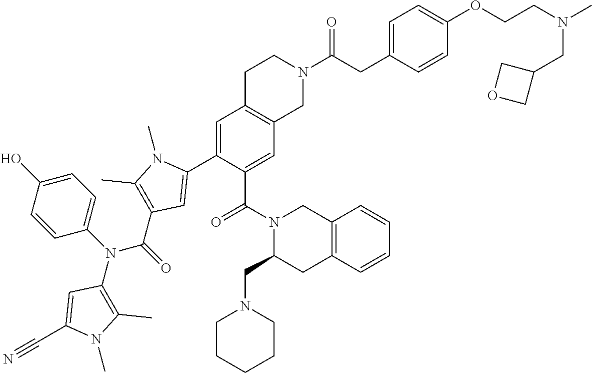 [SMILES: Cc1c(N(C(=O)c2cc(-c3cc4c(cc3C(=O)N3Cc5ccccc5C[C@H]3CN3CCCCC3)CN(C(=O)Cc3ccc(OCCN(C)CC5COC5)cc3)CC4)n(C)c2C)c2ccc(O)cc2)cc(C#N)n1C]

### Step A: 2-[4-[2-[methyl(oxetan-3-ylmethyl)amino]ethoxy]phenyl]acetic acid

Using the General procedure 7d and then General procedure 8, starting from methyl 2-[4-(2-bromoethoxy)phenyl]acetate (250 mg, 0.91 mmol) as the appropriate alkyl halide and N-methyl-1-(oxetan-3-yl)methanamine (185 mg, 2.0 eq.) as the appropriate amine, the desired product was obtained (188 mg, 54%).

¹H NMR (500 MHz, DMSO-d6) δ ppm 11.20 (br., 1H), 7.15 (dm, 2H), 6.86 (dm, 2H), 4.61/4.24 (dd+t, 4H), 4.01 (t, 2H), 3.47 (s, 2H), 3.14 (m, 1H), 2.07 (d, 2H), 2.67 (t, 2H), 2.18 (s, 3H); ¹³C NMR (125 MHz, DMSO-d6) δ ppm 173.5, 157.6, 130.8, 127.5, 114.7, 75.5, 66.2, 61.2, 56.3, 42.9, 40.3, 33.2.

HRMS-ESI (m/z) [M+H]⁺ calcd for C₁₅H₂₂NO₄: 280.1543, found 280.1544.

### Step B: N-(5-cyano-1,2-dimethyl-1H-pyrrol-3-yl)-N-(4-hydroxyphenyl)-1,2-dimethyl-5-(2-{2-[4-(2-{methyl[(oxetan-3-yl)methyl]amino]ethoxy)phenyl]acetyl}-7-[(3S)-3-[(piperidin-1-yl)methyl]-1,2,3,4-tetrahydroisoquinoline-2-carbonyl]-1,2,3,4-tetrahydroisoquinolin-6-yl)-1H-pyrrole-3-carboxamide

Using General procedure 4c, starting from the product of Step A (82 mg, 2.5 eq.) as the appropriate carboxylic acid and Preparation VIIa (100 mg, 0.12 mmol) as the appropriate amine, afforded the title compound (73 mg, 62%).

HRMS-ESI (m/z) [M+H]⁺ calcd for C₆₀H₆₉N₈O₆: 997.5335, found 997.5340.

### Example 105: N-(5-cyano-1,2-dimethyl-1H-pyrrol-3-yl)-5-{2-[2-(2-fluoro-4-{2-[methyl(oxetan-3-yl)amino]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

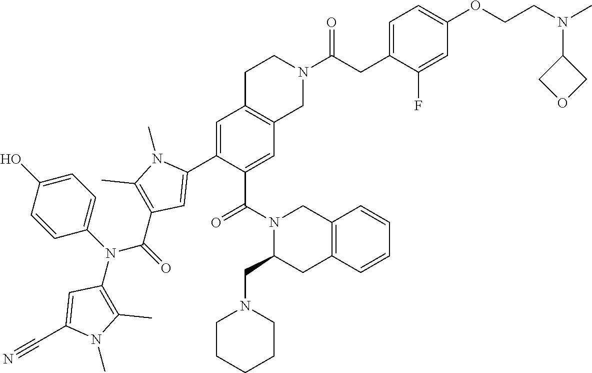 [SMILES: Cc1c(N(C(=O)c2cc(-c3cc4c(cc3C(=O)N3Cc5ccccc5C[C@H]3CN3CCCCC3)CN(C(=O)Cc3ccc(OCCN(C)C5COC5)cc3F)CC4)n(C)c2C)c2ccc(O)cc2)cc(C#N)n1C]

### Step A: 2-[2-fluoro-4-[2-[methyl(oxetan-3-yl)amino]ethoxy]phenyl]acetic acid

Using the General procedure 7d and then General procedure 8, starting from Preparation X (150 mg, 0.55 mmol) as the appropriate alkyl halide and N-methyloxetan-3-amine (90 mg, 2.0 eq.) as the appropriate amine, the desired product was obtained (57 mg, 39%).

¹H NMR (400 MHz, DMSO-d6) δ ppm 12.39 (br., 1H), 7.21 (t, 1H), 6.81 (dd, 1H), 6.73 (dd, 1H), 4.52 (t, 2H), 4.41 (t, 2H), 4.02 (t, 2H), 3.63 (m, 1H), 3.52 (s, 2H), 2.62 (t, 2H), 2.16 (s, 3H).

LCMS-ESI (m/z) [M+H]⁺ calcd for C₁₄H₁₉FNO₄: 284.3, found 284.2.

### Step B: N-(5-cyano-1,2-dimethyl-1H-pyrrol-3-yl)-5-{2-[2-(2-fluoro-4-{2-[methyl(oxetan-3-yl)amino]ethoxy]phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step A (53.3 mg, 2.0 eq.) as the appropriate carboxylic acid and Preparation VIIa (80 mg, 0.09 mmol) as the appropriate amine, afforded the title compound (60 mg, 64%).

HRMS-ESI (m/z) [M+H]⁺ calcd for C₅₉H₆₆FN₈O₆: 1001.5089, found 1001.5079.

### Example 106: N-(5-cyano-1,2-dimethyl-1H-pyrrol-3-yl)-N-(4-hydroxyphenyl)-1,2-dimethyl-5-{2-[2-(4-{2-[methyl(oxetan-3-yl)amino]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-1H-pyrrole-3-carboxamide

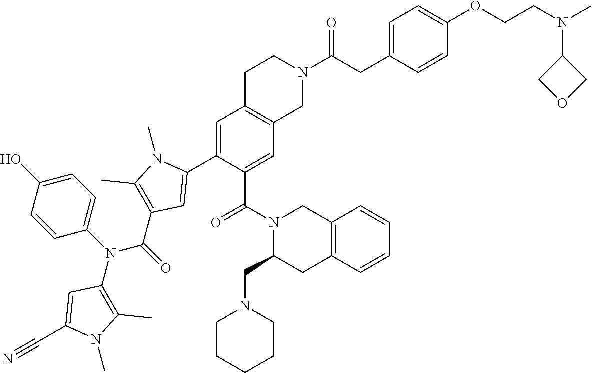 [SMILES: Cc1c(N(C(=O)c2cc(-c3cc4c(cc3C(=O)N3Cc5ccccc5C[C@H]3CN3CCCCC3)CN(C(=O)Cc3ccc(OCCN(C)C5COC5)cc3)CC4)n(C)c2C)c2ccc(O)cc2)cc(C#N)n1C]

Using General procedure 4c, starting from the product of Step A of Example 60 (50 mg, 2.0 eq.) as the appropriate carboxylic acid and Preparation VIIa (80 mg, 0.09 mmol) as the appropriate amine, afforded the title compound (57 mg, 62%).

HRMS-ESI (m/z) [M+H]⁺ calcd for C₅₉H₆₇N₈O₆: 983.5183, found 983.5175.

### Example 107: N-(5-cyano-1,2-dimethyl-1H-pyrrol-3-yl)-5-[2-(2-{2-fluoro-4-[2-(morpholin-4-yl)ethoxyphenyl}acetyl)-7-[(3S)-3-{[(3S)-3-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

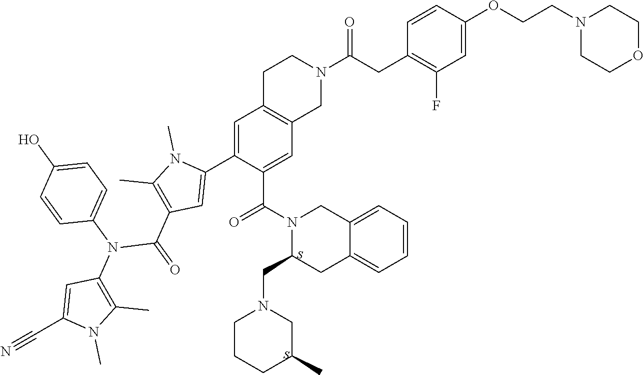

### Step A: (3S)-3-[[(3S)-3-methyl-1-piperidyl]methyl]-1,2,3,4-tetrahydroisoquinoline

(3S)-2-tert-butoxycarbonyl-3,4-dihydro-]H-isoquinoline-3-carboxylic acid (700 mg, 2.5 mmol) and (3S)-3-methylpiperidine hydrochloride (450 mg, 1.4 eq) were reacted using General procedure 5a then the crude intermediate was deprotected using General procedure 6c and then treated according to General procedure 6a to afford the title product (140 mg, 22%).

¹H NMR (400 MHz, DMSO-d6) δ ppm 7.14-6.93 (m, 4H), 3.91 (s, 2H), 2.95 (m, 1H), 2.79/1.52 (d+t, 2H), 2.72/1.92 (d+td, 2H), 2.65/2.37 (dd+dd, 2H), 2.33/2.26 (dd+dd, 2H), 1.64/0.84 (dm+m, 2H), 1.62-1.42 (m, 2H), 1.58 (m, 1H), 0.83 (d, 3H); ¹³C NMR (100 MHz, DMSO-d6) δ ppm 64.5, 62.2, 55.3, 50.9, 48.3, 34.1, 33.2, 31.1, 25.6, 20.1.

HRMS-ESI (m/z) [M+H]⁺ calcd for C₁₆H₂₅N₂: 245.2012, found 245.2016.

### Step B: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(3S)-3-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from Preparation IX (90 mg, 0.10 mmol) as the appropriate carboxylic acid and the product from Step A (28 mg, 1 eq.) as the appropriate amine, afforded the desired product (38.3 mg, 38%).

HRMS-ESI (m/z) [M+H]⁺ calcd for C₆₀H₆₈FN₈O₆: 1015.5240, found 1015.5239.

### Example 108: N-(5-cyano-1,2-dimethyl-1H-pyrrol-3-yl)-5-[2-(2-{2-fluoro-4-[2-((2S or R)-2-methylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 1)

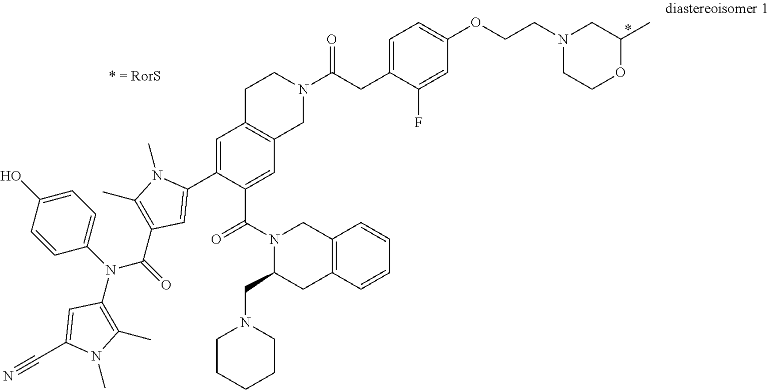 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCOC(C)C5)cc3F)CC4)n(C)c2C)c2ccc(O)cc2)cc(C#N)n1C]

Using General procedure 4c, starting from the product of Step B of Example 75 (23 mg, 2.2 eq.) as the appropriate carboxylic acid and Preparation VIIa (30 mg, 0.04 mmol) as the appropriate amine, afforded the title compound (23 mg, 64%).

HRMS-ESI (m/z) [M+H]⁺ calcd for C₆₀H₆₈FN₈O₆: 1015.5240, found 1015.5251.

### Example 109: N-(5-cyano-1,2-dimethyl-1H-pyrrol-3-yl)-5-[2-(2-{2-fluoro-4-[2-((2R or S)-2-methylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 2)

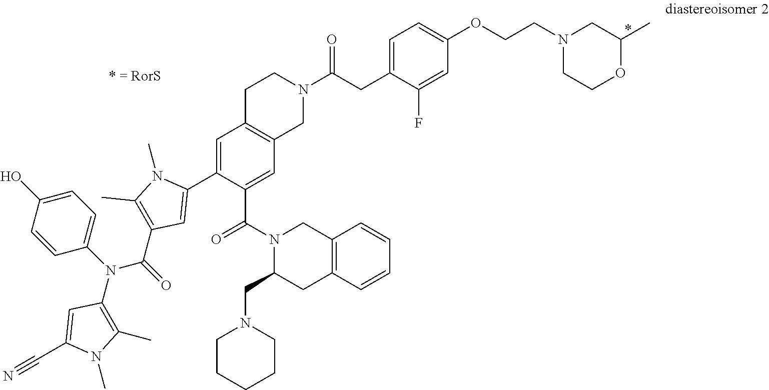 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCOC(C)C5)cc3F)CC4)n(C)c2C)c2ccc(O)cc2)cc(C#N)n1C]

### Step A: 2-[2-fluoro-4-[2-((2R or S)-2-methylmorpholin-4-yl)ethoxy]phenyl]acetic acid (from Enantiomer 2)

Using the General procedure 8 starting from the Enantiomer 2 of Example 75, Step A (390 mg, 1.25 mmol), the desired product was obtained (325 mg, 87%).

¹H NMR (500 MHz, DMSO-d6) δ ppm 12.38 (br., 1H), 7.20 (t, 1H), 6.81 (dd, 1H), 6.73 (dd, 1H), 4.07 (t, 2H), 3.72/3.48 (dm+td, 2H), 3.52 (s, 2H), 3.50 (m, 1H), 2.81/1.78 (dm+dd, 2H), 2.74/2.08 (dm+td, 2H), 2.66 (t, 2H), 1.03 (d, 3H); ¹³C NMR (125 MHz, DMSO-d6) δ ppm 172.5, 161.6, 159.3, 132.7, 114.5, 111.0, 102.2, 71.5, 66.3, 66.1, 60.5, 57.1, 53.3, 34.0, 19.5.

HRMS-ESI (m/z) [M+H]⁺ calcd for C₁₅H₂₁FNO₄: 298.1449, found 298.1450.

### Step B: N-(5-cyano-1,2-dimethyl-1H-pyrrol-3-yl)-5-[2-(2-{2-fluoro-4-[2-((2R or S)-2-methylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 2)

Using General procedure 4c, starting from the product of Step A (87 mg, 2.5 eq.) as the appropriate carboxylic acid and Preparation VIIa (100 mg, 0.12 mmol) as the appropriate amine, afforded the title compound (75 mg, 63%).

HRMS-ESI (m/z) [M+H]⁺ calcd for C₆₀H₆₈FN₈O₆: 1015.5240, found 1015.5246.

### Example 110: N-[5-cyano-1-(difluoromethyl)-2-methylpyrrol-3-yl]-N-(4-hydroxyphenyl)-1,2-dimethyl-5-[2-(2-{2-fluoro-4-[2-(morpholin-4-yl)ethoxyphenyl}acetyl)-7-{[(3S)-3-(pyrrolidin-1-ylmethyl)-3,4-dihydro-1H-isoquinolin-2-yl]carbonyl}-3,4-dihydro-1H-isoquinolin-6-yl]pyrrole-3-carboxamide

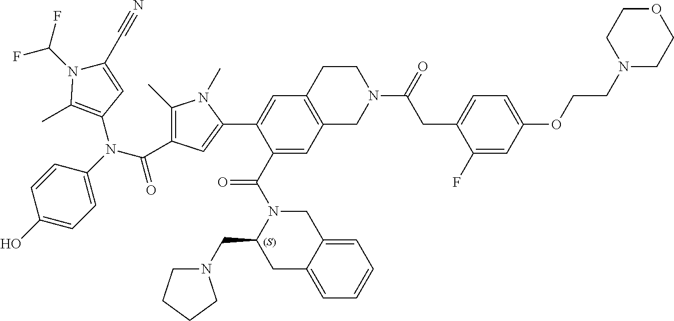

### Step A: tert-butyl 6-(4-{[5-cyano-1-(difluoromethyl)-2-methylpyrrol-3-yl]({4-[(triisopropylsilyl)oxy]phenyl})carbamoyl}-1,5-dimethylpyrrol-2-yl)-7-f[(3S)-3-(pyrrolidin-1-ylmethyl)-3,4-dihydro-1H-isoquinolin-2-yl]carbonyl}-3,4-dihydro-1H-isoquinoline-2-carboxylate

Using General procedure 2a and Preparation IIc (0.8 g, 1.31 mmol, 1 eq.) as the appropriate acid and the product from Example 77, Step A (0.82 g, 1.96 mmol, 1.5 eq.) as the appropriate aniline afforded the title product (0.79 g, 0.78 mmol, 60%).

LRMS calcd for C₅₈H₇₃F₂N₇O₅Si: 1013.5, found 1014.8 [M+H]⁺

### Step B: N-[5-cyano-1-(difluoromethyl)-2-methylpyrrol-3-yl]-1,2-dimethyl-5-(7-f[(3S)-3-(pyrrolidin-1-ylmethyl)-3,4-dihydro-1H-isoquinolin-2-yl]carbonyl}-1,2,3,4-tetrahydroisoquinolin-6-yl)-N-{4-[(triisopropylsilyl)oxy]phenyl}pyrrole-3-carboxamide

Using General procedure 3b and the product from Step A (790 mg, 0.78 mmol, 1 eq.) as the appropriate silyl-derivative afforded the title product (405 mg, 0.44 mmol, 57%).

LRMS calcd for C₅₃H₆₅F₂N₇O₃Si: 913.5, found 914.6 M+H]⁺

### Step C: N-[5-cyano-1-(difluoromethyl)-2-methylpyrrol-3-yl]-N-(4-hydroxyphenyl)-1,2-dimethyl-5-[2-(2-{2-fluoro4-[2-(morpholin-4-yl)ethoxy]phenyl}acetyl)-7-{[(3S)-3-(pyrrolidin-1-ylmethyl)-3,4-dihydro-]H-isoquinolin-2-yl]carbonyl}-3,4-dihydro-1H-isoquinolin-6-yl]pyrrole-3-carboxamide

Using General procedure 4a and the product from Step B (265 mg, 0.29 mmol, 1 eq.) as the appropriate amine and Preparation Vb (90 mg, 0.319 mmol, 1.1 eq.) as the appropriate carboxylic acid afforded an intermediate which was treated according to General procedure 3b with heating at 50° C., to afford the title product (115.8 mg, 0.11 mmol, 39%).

HRMS calcd for C₅₈H₆₁F₃N₈O₆: 1022.4666, found 1023.4739 [M+H]⁺

### Example 111: 5-[2-(2-{2-fluoro-4-[2-(morpholin-4-yl)ethoxy]phenyl}acetyl)-7-{[(3R)-3-methyl-3,4-dihydro-1H-isoquinolin-2-yl]carbonyl}-3,4-dihydro-1H-isoquinolin-6-yl]-N-(5-fluoro-6-methoxypyridin-3-yl)-N-(4-hydroxyphenyl)-1,2-dimethylpyrrole-3-carboxamide

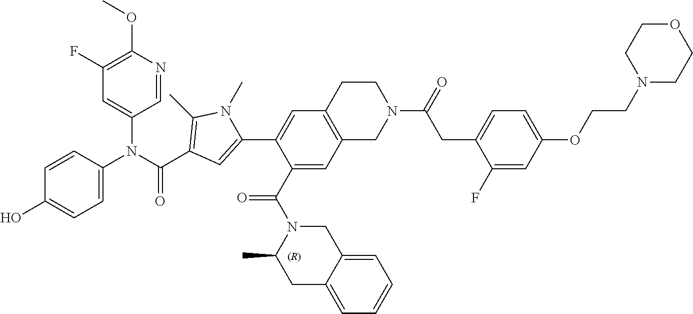

### Step A: tert-butyl 6-[4-({4-[(tert-butyldimethylsilyl)oxy]phenyl}(5-fluoro-6-methoxypyridin-3-yl)carbamoyl)-1,5-dimethylpyrrol-2-yl]-7-{[(3R)-3-methyl-3,4-dihydro-1H-isoquinolin-2-yl]carbonyl}-3,4-dihydro-1H-isoquinoline-2-carboxylate

Using General procedure 2a and Preparation Ha (350 mg, 0.64 mmol, 1 eq.) as the appropriate acid and the product from Example 72, Step A (337 mg, 0.97 mmol, 1.5 eq.) as the appropriate aniline afforded the title product (493 mg, 0.56 mmol, 88%).

LRMS calcd for C₅₀H₆₀FN₅O₆Si: 873.4, found 874.7 [M+H]⁺

### Step B: N-(5-fluoro-6-methoxypyridin-3-yl)-N-(4-hydroxyphenyl)-1,2-dimethyl-5-(7-{[(3R)-3-methyl-3,4-dihydro-1H-isoquinolin-2-yl]carbonyl}-1,2,3,4-tetrahydroisoquinolin-6-yl)pyrrole-3-carboxamide

Using General procedure 3b and the product from Step A (493 mg, 0.56 mmol, 1 eq.) as the appropriate silyl-derivative afforded the title product (188 mg, 0.28 mmol, 51%).

LRMS calcd for C₃₉H₃₈FN₅O₄: 659.3, found 660.4 [M+H]⁺

### Step C: 5-[2-(2-{2-fluoro-4-[2-(morpholin-4-yl)ethoxy]phenyl}acetyl)-7-{[(3R)-3-methyl-3,4-dihydro-1H-isoquinolin-2-yl]carbonyl}-3,4-dihydro-1H-isoquinolin-6-yl]-N-(5-fluoro-6-methoxypyridin-3-yl)-N-(4-hydroxyphenyl)-1,2-dimethylpyrrole-3-carboxamide

Using General procedure 4a and the product from Step B (188 mg, 0.28 mmol, 1 eq.) as the appropriate amine and Preparation Vb (121 mg, 0.43 mmol, 1.5 eq.) as the appropriate carboxylic acid afforded the title product (33 mg, 0.04 mmol, 13%).

HRMS calcd for C₅₃H₅₄F₂N₆O₇: 924.4022, found 925.4098 [M+H]⁺.

### Example 112: N-(5-cyano-1,2-dimethylpyrrol-3-yl)-5-{2-[2-(4-{2-[4-(2,2-difluoroethyl)piperazin-1-yl]ethoxy}phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

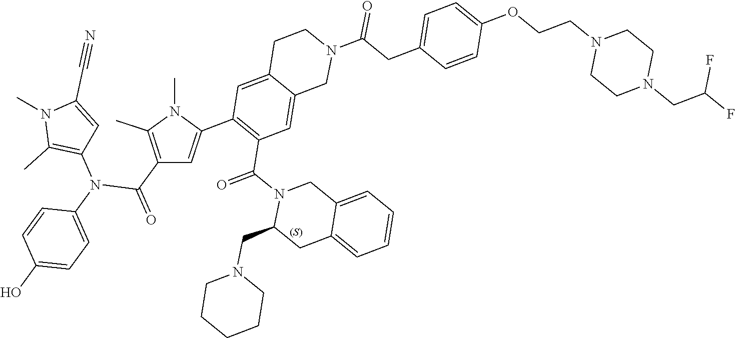

### Step A: methyl 2-[4-(2-chloroethoxy)phenyl]acetate

Using General procedure 7b and methyl 4-hydroxyphenylacetate (500 mg, 3.01 mmol, 1 eq.) as the appropriate phenol and 2-chloroethanol (0.3 mL, 4.51 mmol, 1.5 eq.) as the appropriate alcohol afforded the title product (587.5 mg, 2.57 mmol, 85%).

¹H NMR (400 MHz, DMSO-d6) δ ppm: 7.21-7.15 (m, 2H), 6.94-6.88 (m, 2H), 4.26-4.20 (m, 2H), 3.96-3.90 (m, 2H), 3.63-3.57 (m, 5H).

### Step B: methyl 2-(4-{2-[4-(2,2-difluoroethyl)piperazin-1-yl]ethoxy]phenyl)acetate

A mixture of the product from Step A (280 mg, 1.22 mmol, 1 eq.), K₂CO₃ (677 mg, 4.9 mmol, 4 eq.), NaI (92 mg, 0.61 mmol, 0.5 eq.) and 1-(2,2-difluoroethyl)piperazine (202 mg, 1.35 mmol, 1.1 eq.) in MeCN (20 mL) was refluxed for 18 h. The mixture was allowed to cool to rt, filtered and washed with EtOAc. The filtrate was concentrated in vacuo and purified by automated flash chromatography eluting with a gradient of 0-100% EtOAc in heptane to afford the title product (113 mg, 0.33 mmol, 27%).

LRMS calcd for C₁₇H₂₄F₂N₂O₃: 342.2, found 343.2 [M+H]⁺

¹H NMR (400 MHz, DMSO-d6) δ ppm: 7.18-7.12 (m, 2H), 6.90-6.84 (m, 2H), 6.11 (tt, J=55.8, 4.4 Hz, 1H), 4.03 (t, J=5.8 Hz, 2H), 3.59 (s, 3H), 3.59 (s, 2H), 2.76-2.62 (m, 4H), 2.57-2.41 (br m, 8H).

### Step C: (4-{2-[4-(2,2-difluoroethyl)piperazin-1-yl]ethoxy]phenyl)acetic acid

Using General procedure 8 and the product from Step B (113 mg, 0.33 mmol, 1 eq.) as the appropriate ester afforded the title product (89.3 mg, 0.27 mmol, 82%).

LRMS calcd for C₁₆H₂₂F₂N₂O₃: 328.2, found 329.4 [M+H]⁺

¹H NMR (400 MHz, DMSO-d6) δ ppm: 12.24 (s, 1H), 7.25-7.09 (m, 2H), 7.00-6.82 (m, 2H), 6.13 (tt, J=55.6, 4.3 Hz, 1H), 4.47-3.91 (br m, 2H), 3.74-2.25 (br m, 14H).

### Step D: N-(5-cyano-1,2-dimethylpyrrol-3-yl)-5-{2-[2-(4-{2-[4-(2,2-difluoroethyl)piperazin-1-yl]ethoxy]phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

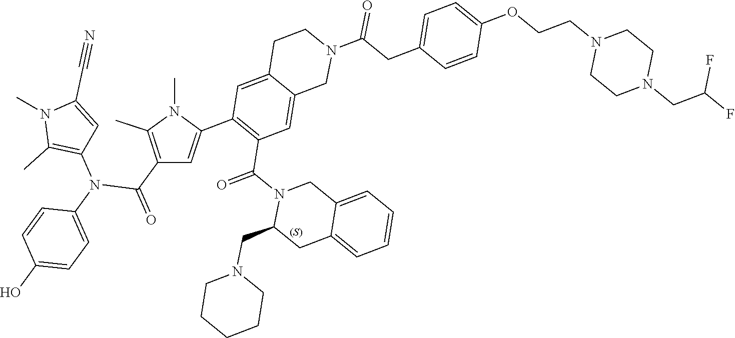

Using General procedure 4a and Preparation IVb (125 mg, 0.17 mmol, 1 eq.) as the appropriate amine and the product from Step C (61 mg, 0.19 mmol, 1.1 eq.) as the appropriate carboxylic acid afforded the title product (99.4 mg, 0.1 mmol, 56%).

HRMS calcd for C₆₁H₆₉F₂N₉O₅: 1045.5390, found 1046.5466 [M+H]⁺

### Example 113: N-(5-cyano-1,2-dimethyl-1H-pyrrol-3-yl)-N-(4-hydroxyphenyl)-1,2-dimethyl-5-[2-(2-{2-methyl-4-[2-(morpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

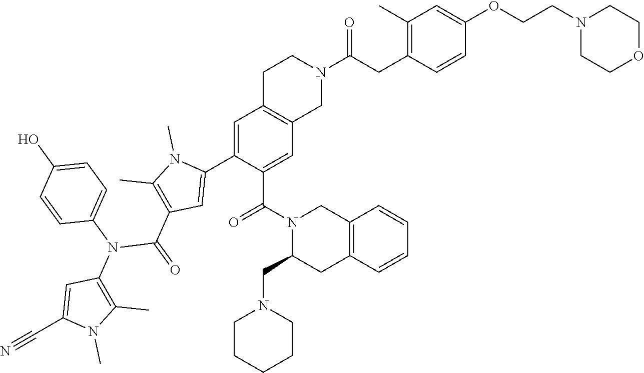 [SMILES: Cc1cc(OCCN2CCOCC2)ccc1CC(=O)N1CCc2cc(-c3cc(C(=O)N(c4ccc(O)cc4)c4cc(C#N)n(C)c4C)c(C)n3C)c(C(=O)N3Cc4ccccc4C[C@H]3CN3CCCCC3)cc2C1]

### Step A: 2-[2-methyl-4-(2-morpholinoethoxy)phenyl]acetic acid

Using the General procedure 7b and then General procedure 8, starting from methyl 2-(4-hydroxy-2-methylphenyl)acetate (257 mg, 1.43 mmol) as the appropriate phenol and 2-morpholinoethanol (281 mg, 1.5 eq.) as the appropriate alcohol, followed with a purification step by automated flash chromatography using DCM and MeOH (1% TEA) as eluents afforded the title product (490 mg, 90%, TEA salt).

¹H NMR (500 MHz, DMSO-d6) δ ppm 7.06 (d, 1H), 6.76 (d, 1H), 6.69 (dd, 1H), 4.07/3.69 (brt+br, 4H), 3.62/2.73 (br+br, 4H), 3.6 (br, 2H), 3.48 (s, 2H), 2.52 (br, 2H), 2.19 (s, 3H); ¹³C NMR (125 MHz, DMSO-d6) δ ppm 131.7, 116.5, 112, 66.4, 65.3, 57.4, 53.9, 38.4, 19.8.

HRMS-ESI (m/z) [M+H]⁺ calcd for C₁₅H₂₂NO₄: 280.1543, found 280.1544.

### Step B: N-(5-cyano-1,2-dimethyl-1H-pyrrol-3-yl)-N-(4-hydroxyphenyl)-1,2-dimethyl-5-[2-(2-{2-methyl-4-[2-(morpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

Using General procedure 4c, starting from the product of Step A (90 mg, 2.5 eq.) as the appropriate carboxylic acid and Preparation VIIa (80 mg, 0.09 mmol) as the appropriate amine, afforded the title compound (65 mg, 69%).

HRMS-ESI (m/z) [M+H]⁺ calcd for C₆₀H₆₉N₈O₆: 997.5338, found 997.5337.

### Example 114: N-(5-cyano-1,2-dimethyl-1H-pyrrol-3-yl)-N-(4-hydroxyphenyl)-5-[2-(2-{2-methoxy-4-[2-(morpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1,2-dimethyl-1H-pyrrole-3-carboxamide

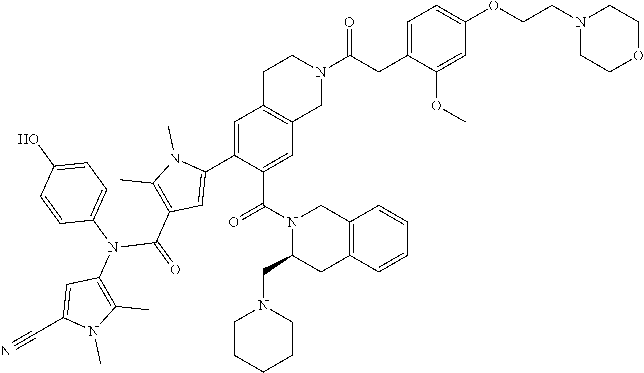 [SMILES: COc1cc(OCCN2CCOCC2)ccc1CC(=O)N1CCc2cc(-c3cc(C(=O)N(c4ccc(O)cc4)c4cc(C#N)n(C)c4C)c(C)n3C)c(C(=O)N3Cc4ccccc4C[C@H]3CN3CCCCC3)cc2C1]

### Step A: 2-[2-methoxy-4-(2-morpholinoethoxy)phenyl]acetic acid

Using the General procedure 7b and then General procedure 8, starting from methyl 2-(4-hydroxy-2-methoxyphenyl)acetate (180 mg, 0.92 mmol) as the appropriate phenol and 2-morpholinoethanol (181 mg, 1.5 eq.) as the appropriate alcohol, followed with a purification step by automated flash chromatography using DCM and MeOH (1% TEA) as eluents afforded the title product (248 mg, 92%).

¹H NMR (500 MHz, DMSO-d6) δ ppm 12.00/10.04 (brs, 2H), 7.04 (d, 1H), 6.53 (d, 1H), 6.46 (dd, 1H), 4.08 (brt, 2H), 3.73 (s, 3H), 3.59 (brm, 4H), 3.4 (s, 2H), 2.70-2.30 (brs, 4H), 2.69 (brs, 2H); ¹³C NMR (125 MHz, DMSO-d6) δ ppm 131.7, 105.4, 92.2, 66.6, 65.7, 57.5, 55.9, 54.1, 35.2.

HRMS-ESI (m/z) [M+H]⁺ calcd for C₁₅H₂₂NO₅: 296.1492, found 296.1493.

### Step B: N-(5-cyano-1,2-dimethyl-1H-pyrrol-3-yl)-N-(4-hydroxyphenyl)-5-[2-(2-{2-methoxy-4-[2-(morpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1,2-dimethyl-1H-pyrrole-3-carboxamide

Using General procedure 4c, starting from the product of Step A (56 mg, 2 eq.) as the appropriate carboxylic acid and Preparation VIIa (80 mg, 0.09 mmol) as the appropriate amine, afforded the title product (64 mg, 67%).

HRMS-ESI (m/z) [M+H]⁺ calcd for C₆₀H₆₉N₈O₇: 1013.5284, found 1013.5285.

### Example 115: N-(5-cyano-1,2-dimethyl-1H-pyrrol-3-yl)-5-[2-(2-{2-fluoro-4-[2-(4-fluoropiperidin-1-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

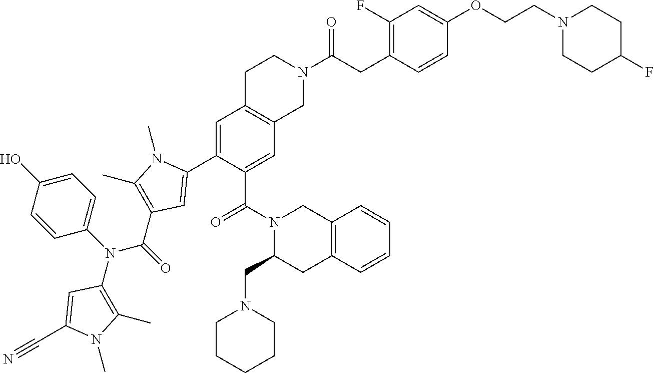 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCC(F)CC5)cc3F)CC4)n(C)c2C)c2ccc(O)cc2)cc(C#N)n1C]

### Step A: 2-[2-fluoro-4-[2-(4-fluoro-1-piperidyl)ethoxy]phenyl]acetic acid

Using the General procedure 7d and then General procedure 8, starting from methyl 2-[4-(2-bromoethoxy)-2-fluoro-phenyl]acetate (200 mg, 0.68 mmol) as the appropriate alkyl halide and 4-fluoropiperidine (200 mg, 2.1 eq.) as the appropriate amine, followed with a purification step by automated flash chromatography using DCM and MeOH (1% TEA) as eluents afforded the title product (170 mg, 88%).

¹H NMR (500 MHz, DMSO-d6) δ ppm 7.19 (t, 1H), 6.79 (dd, 1H), 6.72 (dd, 1H), 4.66 (dm, 1H), 4.05 (t, 2H), 3.48 (s, 2H), 2.68 (t, 2H), 2.61/2.39 (br+br., 4H), 1.92-1.61 (m, 4H); ¹³C NMR (125 MHz, DMSO-d6) δ ppm 172.7, 161.5, 159.1, 132.6, 115, 110.9, 102.2, 88.9, 66.5, 56.7, 50.0, 34.4, 31.7.

HRMS-ESI (m/z) [M+H]⁺ calcd for C₁₅H₂₀F₂NO₃: 300.1406, found 300.1407.

### Step B: N-(5-cyano-1,2-dimethyl-1H-pyrrol-3-yl)-5-[2-(2-{2-fluoro-4-[2-(4-fluoropiperidin-1-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from the product of Step A (75 mg, 2 eq.) as the appropriate carboxylic acid and Preparation VIIa (80 mg, 0.09 mmol) as the appropriate amine, afforded the title compound (59 mg, 62%).

HRMS-ESI (m/z) [M+H]⁺ calcd for C₆₀H₆₇F₂N₈O₅: 1017.5197, found 1017.5201.

### Example 116: N-(5-cyano-1,2-dimethyl-1H-pyrrol-3-yl)-5-{2-[2-(4-{2-[(2-fluoroethyl)(methyl)amino]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

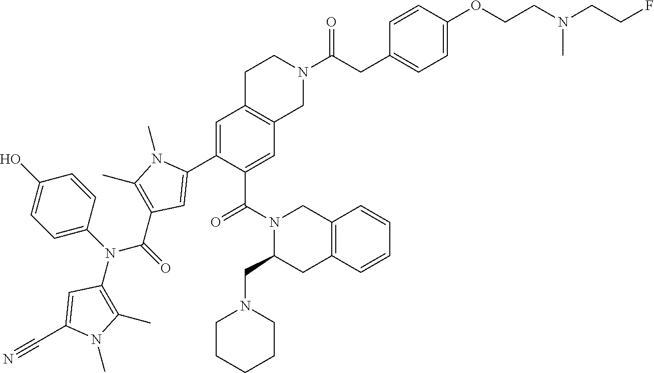 [SMILES: Cc1c(N(C(=O)c2cc(-c3cc4c(cc3C(=O)N3Cc5ccccc5C[C@H]3CN3CCCCC3)CN(C(=O)Cc3ccc(OCCN(C)CCF)cc3)CC4)n(C)c2C)c2ccc(O)cc2)cc(C#N)n1C]

### Step A: 2-[4-[2-[2-fluoroethyl(methyl)amino]ethoxy]phenyl]acetic acid

Using the General procedure 7d and then General procedure 8, starting from methyl 2-[4-(2-bromoethoxy)phenyl]acetate (300 mg, 1.10 mmol) as the appropriate alkyl halide and 2-fluoro-N-methyl-ethanamine, hydrogen chloride (1:1) (300 mg, 1.6 eq.) as the appropriate amine, the desired product was obtained (260 mg, 88%).

¹H NMR (500 MHz, DMSO-d6) δ ppm 7.15 (dm, 2H), 6.87 (dm, 2H), 4.55 (dt, 2H), 4.05 (t, 2H), 3.48 (s, 2H), 2.85/2.78 (br+br., 2H), 2.85 (br., 2H), 2.36 (br., 3H); ¹³C NMR (125 MHz, DMSO-d6) δ ppm 173.4, 157.6, 130.9, 127.5, 114.7, 82.4, 65.9, 57.3, 56.3, 42.9, 40.3.

HRMS-ESI (m/z) [M+H]⁺ calcd for C₁₃H₁₉FNO₃: 256.1343, found 256.1345.

### Step B: N-(5-cyano-1,2-dimethyl-1H-pyrrol-3-yl)-5-{2-[2-(4-{2-[(2-fluoroethyl)(methyl)amino]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step A (100 mg, 3.0 eq.) as the appropriate carboxylic acid and Preparation VIIa (80 mg, 0.09 mmol) as the appropriate amine, afforded the title compound (66 mg, 72%).

HRMS-ESI (m/z) [M+H]⁺ calcd for C₅₈H₆₆FN₈O₅: 973.5140, found 973.5140.

### Example 117: N-(5-cyano-1,2-dimethyl-1H-pyrrol-3-yl)-5-{2-[2-(2-fluoro-4-{2-[4-(2-fluoroethyl)piperazin-1-yl]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

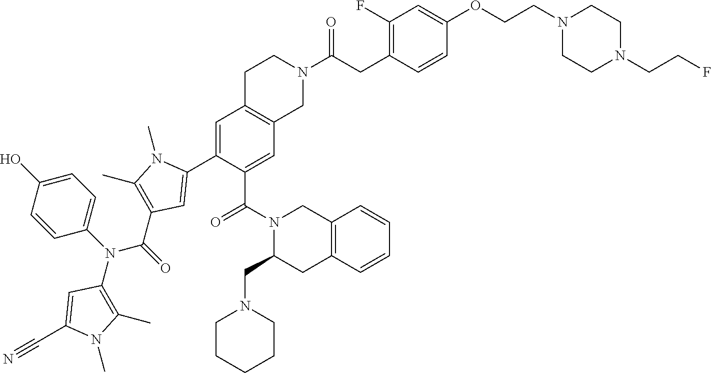 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCN(CCF)CC5)cc3F)CC4)n(C)c2C)c2ccc(O)cc2)cc(C#N)n1C]

### Step A: 2-[2-fluoro-4-[2-[4-(2-fluoroethyl)piperazin-1-yl]ethoxy]phenyl]acetic acid

Using the General procedure 7d and then General procedure 8, starting from Preparation X (150 mg, 0.55 mmol) as the appropriate alkyl halide and 1-(2-fluoroethyl)piperazine (170 mg, 2.5 eq.) as the appropriate amine, followed with a step of salt formation using hydrogen chloride in Et₂O afforded the dihydrochloride of the desired product (168 mg, 89%).

¹H NMR (400 MHz, DMSO-d6) δ ppm 7.27 (t, 1H), 6.90 (dd, 1H), 6.81 (dd, 1H), 4.84 (brd., 2H), 4.39 (br., 2H), 4.20-2.80 (br., 12H), 3.55 (s, 2H); ¹³C NMR (100 MHz, DMSO-d6) δ ppm 172.4, 132.8, 111.1, 102.8, 79.4, 63.5, 34.0.

HRMS-ESI (m/z) [M+H]⁺ calcd for C₁₆H₂₃F₂N₂O₃: 329.1671, found 329.1671.

### Step B: N-(5-cyano-1,2-dimethyl-1H-pyrrol-3-yl)-5-{2-[2-(2-fluoro-4-{2-[4-(2-fluoroethyl)piperazin-1-yl]ethoxy]phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step A (69 mg, 2 eq.) as the appropriate carboxylic acid and Preparation VIIa (80 mg, 0.09 mmol) as the appropriate amine, afforded the title compound (50 mg, 51%).

HRMS-ESI (m/z) [M+H]⁺ calcd for C₆₁H₇₀F₂N₉O₅: 1046.5462, found 1046.5462.

### Example 118: N-(5-cyano-1,2-dimethyl-1H-pyrrol-3-yl)-N-(4-hydroxyphenyl)-1,2-dimethyl-5-[2-(2-{6-[2-(oxan-4-yl)ethoxy]pyridin-3-yl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

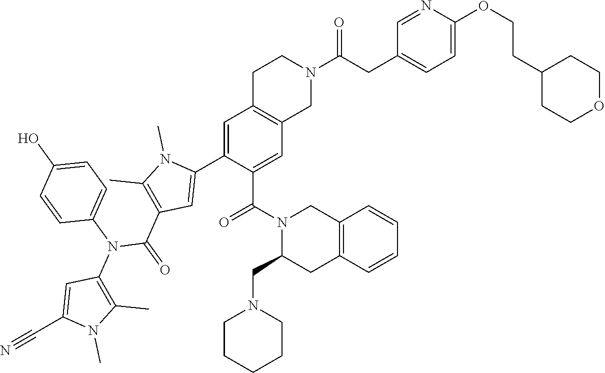 [SMILES: Cc1c(N(C(=O)c2cc(-c3cc4c(cc3C(=O)N3Cc5ccccc5C[C@H]3CN3CCCCC3)CN(C(=O)Cc3ccc(OCCC5CCOCC5)nc3)CC4)n(C)c2C)c2ccc(O)cc2)cc(C#N)n1C]

### Step A: 2-[6-(2-tetrahydropyran-4-ylethoxy)-3-pyridyl]acetic acid

2-(6-chloro-3-pyridyl)acetic acid (163 mg, 0.95 mmol), 2-tetrahydropyran-4-ylethanol (239 mg, 2 eq.) and sodium tert-butoxide (280 mg, 3 eq.) were dissolved in 1,4-dioxane (1 mL/mmol), then the reaction mixture was heated in a sealed tube at 150° C. under microwave irradiation for 20 minutes. Then the mixture was purified directly by reverse phase automated flash chromatography using water and MeCN as eluents to obtain the title product (158 mg, 63%).

¹H NMR (500 MHz, DMSO-d6) δ ppm 7.99 (d, 1H), 7.57 (dd, 1H), 6.74 (d, 1H), 4.27 (t, 2H), 3.81/3.27 (dd+td, 4H), 3.51 (s, 2H), 1.65 (m, 1H), 1.64 (q, 2H), 1.60/1.20 (d+dd, 4H); ¹³C NMR (125 MHz, DMSO-d6) δ ppm 173.2, 162.7, 147.4, 140.9, 124.3, 110.5, 67.5, 63.3, 37.3, 35.9, 33.1, 32.0.

HRMS-ESI (m/z) [M+H]⁺ calcd for C₁₄H₂₀NO₄: 266.1387, found 266.1386.

### Step B: N-(5-cyano-1,2-dimethyl-1H-pyrrol-3-yl)-N-(4-hydroxyphenyl)-1,2-dimethyl-5-[2-(2-{6-[2-(oxan-4-yl)ethoxy]pyridin-3-yl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

Using General procedure 4c, starting from the product of Step A (150 mg, 6.0 eq.) as the appropriate carboxylic acid and Preparation VIIa (80 mg, 0.09 mmol) as the appropriate amine, afforded the title product (62 mg, 67%).

HRMS-ESI (m/z) [M+H]⁺ calcd for C₅₉H₆₆N₈O₆: 983.5178, found 983.5181.

### Example 119: N-(5-cyano-1,2-dimethyl-1H-pyrrol-3-yl)-N-(4-hydroxyphenyl)-1,2-dimethyl-5-[2-(2-{4-[2-(4-methylpiperazin-1-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

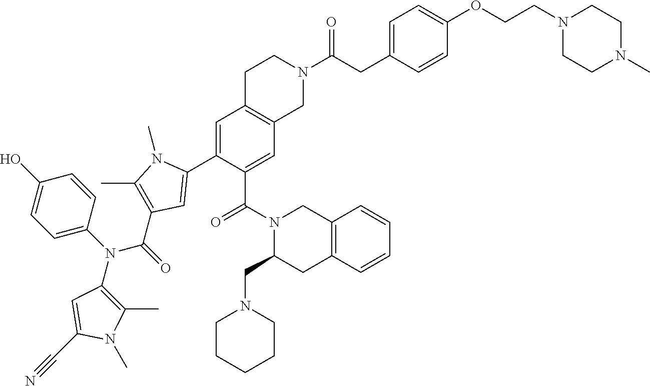 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCN(C)CC5)cc3)CC4)n(C)c2C)c2ccc(O)cc2)cc(C#N)n1C]

### Step A: 2-[4-[2-(4-methylpiperazin-1-yl)ethoxy]phenyl]acetic acid

Using the General procedure 7b and then General procedure 8, starting from methyl 2-(4-hydroxyphenyl)acetate (1.00 g, 6.02 mmol) as the appropriate phenol and 2-(4-methylpiperazin-1-yl)ethanol (0.95 g, 1.1 eq.) as the appropriate alcohol, the desired product was obtained (940 mg, 56%).

¹H NMR (500 MHz, DMSO-d6) δ ppm 12.42/12.18 (brs, 2H), 7.20 (m, 2H), 6.95 (m, 2H), 4.42 (brt, 2H), 3.95-3.45 (brm, 8H), 3.60 (brm, 2H), 3.50 (s, 2H), 2.81 (brs, 3H); ¹³C NMR (125 MHz, DMSO-d6) δ ppm 130.9, 115.0, 62.7, 55.0, 42.6, 40.2.

HRMS-ESI (m/z) [M+H]⁺ calcd for C₁₅H₂₃N₂O₃: 279.1703, found 279.1709.

### Step B: N-(5-cyano-1,2-dimethyl-1H-pyrrol-3-yl)-N-(4-hydroxyphenyl)-1,2-dimethyl-5-[2-(2-{4-[2-(4-methylpiperazin-1-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

Using General procedure 4c, starting from the product of Step A (59 mg, 2.0 eq.) as the appropriate carboxylic acid and Preparation VIIa (90 mg, 0.11 mmol) as the appropriate amine, afforded the title compound (74 mg, 70%).

HRMS-ESI (m/z) [M+H]⁺ calcd for C₆₀H₇₀N₉O₅: 996.5490, found 996.5495.

### Example 120: 5-{7-[(3S)-3-{[cis-3-azabicyclo[3.1.0]hexan-3-yl]methyl}-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

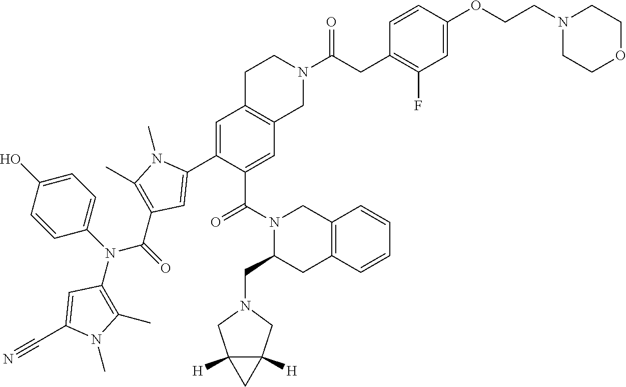 [SMILES: Cc1c(N(C(=O)c2cc(-c3cc4c(cc3C(=O)N3Cc5ccccc5C[C@H]3CN3C[C@H]5C[C@H]5C3)CN(C(=O)Cc3ccc(OCCN5CCOCC5)cc3F)CC4)n(C)c2C)c2ccc(O)cc2)cc(C#N)n1C]

### Step A: benzyl (3S)-3-[cis-3-azabicyclo[3.1.0]hexane-3-carbonyl]-3,4-dihydro-1H-isoquinoline-2-carboxylate

Using the General procedure 5a, starting from (3S)-2-benzyloxycarbonyl-3,4-dihydro-]H-isoquinoline-3-carboxylic acid (934 mg, 3.00 mmol) as the appropriate carboxylic acid and cis-3-azabicyclo[3.1.0]hexane hydrochloride (431 mg, 1.2 eq.) as the appropriate amine, afforded the desired product (873 mg, 76%).

¹H NMR (400 MHz, DMSO-d6) δ ppm 7.46-7.1 (m, 9H), 5.17-4.91 (m, 2H), 4.87-4.51 (m, 1H), 4.83-4.25 (m, 2H), 3.84-3.08 (m, 4H), 3.19-2.72 (m, 2H), 1.68-1.36 (br., 2H), 0.76-−0.57 (br., 2H).

HRMS-ESI (m/z) [M+H]⁺ calcd for C₂₃H₂₅N₂O₃: 377.1860, found 377.1862.

### Step B: [cis-3-azabicyclo[3.1.0]hexan-3-yl]-[(3S)-1,2,3,4-tetrahydroisoquinolin-3-yl]methanone

Using the General procedure 6b, starting from the solution of the product from Step A (893 mg, 2.37 mmol) in EtOAc (50 mL) afforded the desired product (570 mg, 98%).

¹H NMR (500 MHz, DMSO-d6) δ ppm 7.14-6.95 (m, 4H), 3.96-3.8 (d+d, 2H), 3.75-3.21 (m, 2H), 3.61 (m, 1H), 2.86-2.61 (m, 2H), 2.36 (brs, 1H), 1.6/1.54 (m, 1H), 0.69/0.08 (m+m, 2H).

HRMS-ESI (m/z) [M+H]⁺ calcd for C₁₅H₁₉N₂O: 243.1492, found 243.1493.

### Step C: (3S)-3-[[cis-3-azabicyclo[3.1.0]hexan-3-yl]methyl]-1,2,3,4-tetrahydroisoquinoline

The solution of the product from Step B (560 mg, 2.31 mmol) was dissolved in THE (20 mL) and treated with 1M solution of LiAlH₄ in THE (3.0 mL, 1.3 eq.) followed by the General procedure 6a to give the title compound (75 mg, 13%).

¹H NMR (500 MHz, DMSO-d6) δ ppm 7.13-6.95 (m, 4H), 3.91 (s, 2H), 2.98/2.92/2.34/2.23 (d+d/d+d, 4H), 2.86 (m, 1H), 2.65/2.38 (dd+dd, 2H), 2.5/2.38 (m+m, 2H), 1.35 (m, 2H), 0.68/0.31 (m+m, 2H); ¹³C NMR (125 MHz, DMSO-d6) δ ppm 60.4, 55.8/54.8, 52.5, 48.3, 33.9, 15.6/15.4, 7.

HRMS-ESI (m/z) [M+H]⁺ calcd for C₁₅H₂₁N₂: 229.1699, found 229.1699.

### Step D: 5-{7-[(3S)-3-{[cis-3-azabicyclo[3.1.0]hexan-3-yl]methyl}-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

Using General procedure 4c, starting from Preparation IX (80 mg, 0.089 mmol) as the appropriate carboxylic acid and the product of Step C (70 mg, 3.5 eq.) as the appropriate amine, afforded the title compound (22 mg, 24%).

HRMS-ESI (m/z) [M+H]⁺ calcd for C₅₉H₆₃FN₈O₆: 998.4855, found 999.4929.

### Example 121: N-(5-cyano-1,2-dimethyl-1H-pyrrol-3-yl)-5-[2-(2-{4-[2-(2,2-dimethylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

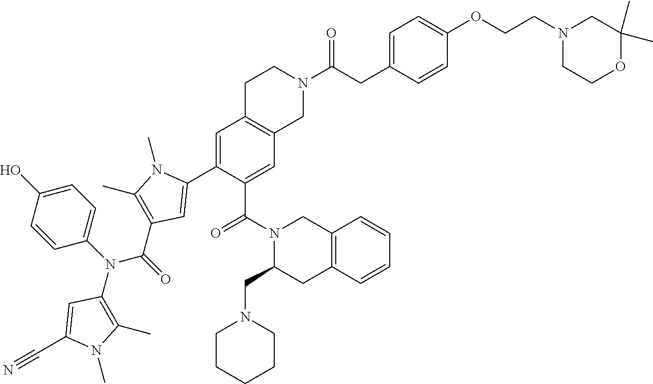 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCOC(C)(C)C5)cc3)CC4)n(C)c2C)c2ccc(O)cc2)cc(C#N)n1C]

### Step A: 2-[4-[2-(2,2-dimethylmorpholin-4-yl)ethoxy]phenyl]acetic acid

Using the General procedure 7d and then General procedure 8, starting from methyl 2-[4-(2-bromoethoxy)phenyl]acetate (250 mg, 0.91 mmol) as the appropriate alkyl halide and 2,2-dimethylmorpholine (126 mg, 1.2 eq.) as the appropriate amine, the desired product was obtained (101 mg, 38%).

¹H NMR (500 MHz, DMSO-d6) δ ppm 10.70 (brs, 1H), 7.15 (m, 2H), 6.88 (m, 2H), 4.05 (t, 2H), 3.58 (t, 2H), 3.48 (s, 2H), 2.63 (t, 2H), 2.39 (m, 2H), 2.26 (s, 2H), 1.14 (s, 6H).

LCMS-ESI (m/z) [M+H]⁺ calcd for C₁₆H₂₄NO₄: 294.2, found 294.2.

### Step B: N-(5-cyano-1,2-dimethyl-1H-pyrrol-3-yl)-5-[2-(2-{4-[2-(2,2-dimethylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from the product of Step A (78 mg, 2.5 eq.) as the appropriate carboxylic acid and Preparation VIIa (90 mg, 0.11 mmol) as the appropriate amine, afforded the title compound (29 mg, 27%).

HRMS-ESI (m/z) [M+H]⁺ calcd for C₆₁H₇₁N₈O₆: 1011.5490, found 1011.5485.

### Example 122: N-(5-cyano-1,2-dimethyl-1H-pyrrol-3-yl)-5-{2-[2-(4-{2-[4-(2-fluoroethyl)piperazin-1-yl]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

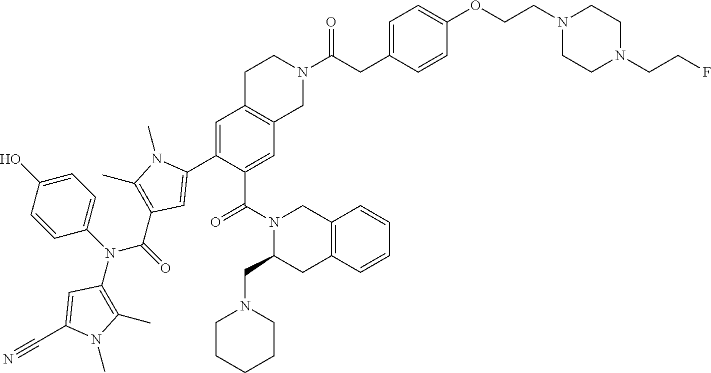 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCN(CCF)CC5)cc3)CC4)n(C)c2C)c2ccc(O)cc2)cc(C#N)n1C]

### Step A: 2-[4-[2-[4-(2-fluoroethyl)piperazin-1-yl]ethoxy]phenyl]acetic acid, dihydrochloride

Using the General procedure 7d and then General procedure 8, starting from methyl 2-[4-(2-bromoethoxy)phenyl]acetate (150 mg, 0.55 mmol) as the appropriate alkyl halide and 1-(2-fluoroethyl)piperazine (181 mg, 2.5 eq.) as the appropriate amine, the desired product was obtained (180 mg, 86%).

¹H NMR (500 MHz, DMSO-d6) δ ppm 12.18 (brs, 1H), 7.15 (m, 2H), 6.87 (m, 2H), 4.51 (dt, 2H), 4.04 (t, 2H), 3.47 (s, 2H), 2.68 (brm, 2H), 2.65-2.30 (brs, 8H), 2.57 (dm, 2H); ¹³C NMR (125 MHz, DMSO-d6) δ ppm 130.8, 114.7, 82.3, 65.8, 57.9, 57.0, 40.2.

HRMS-ESI (m/z) [M+H]⁺ calcd for C₁₆H₂₄FN₂O₃: 311.1766, found 311.1767.

### Step B: N-(5-cyano-1,2-dimethyl-1H-pyrrol-3-yl)-5-{2-[2-(4-{2-[4-(2-fluoroethyl)piperazin-1-yl]ethoxy]phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step A (72 mg, 2.0 eq.) as the appropriate carboxylic acid and Preparation VIIa (80 mg, 0.09 mmol) as the appropriate amine, afforded the title compound (64 mg, 66%).

HRMS-ESI (m/z) [M+H]⁺ calcd for C₆₁H₇₁FN₉O₅: 1028.5562, found 1028.5558.

### Example 123: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(2R)-2-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

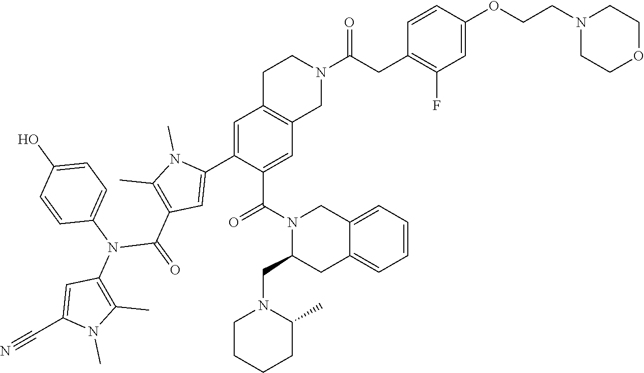 [SMILES: Cc1c(N(C(=O)c2cc(-c3cc4c(cc3C(=O)N3Cc5ccccc5C[C@H]3CN3CCCC[C@H]3C)CN(C(=O)Cc3ccc(OCCN5CCOCC5)cc3F)CC4)n(C)c2C)c2ccc(O)cc2)cc(C#N)n1C]

### Step A: (3S)-3-[[(2R)-2-methyl-1-piperidyl]methyl]-1,2,3,4-tetrahydroisoquinoline

(3S)-2-tert-butoxycarbonyl-3,4-dihydro-1H-isoquinoline-3-carboxylic acid (700 mg, 2.5 mmol) and (2R)-2-methylpiperidine (350 mg, 1.4 eq) were reacted using General procedure 5a then the crude intermediate was deprotected using General procedure 6c and then treated according to General procedure 6a to afford the title product (163 mg, 27%).

¹H NMR (400 MHz, DMSO-d6) δ ppm 7.13-6.95 (m, 4H), 3.90 (s, 2H), 2.85 (m, 1H), 2.76/2.16 (m+m, 2H), 2.7/2.34 (dd+dd, 2H), 2.62/2.15 (dd+dd, 2H), 2.33 (m, 1H), 1.66-1.16 (m, 6H), 0.99 (d, 3H); ¹³C NMR (100 MHz, DMSO-d6) δ ppm 60.1, 56.8, 52.5, 52.3, 48.4, 34.2, 18.4.

HRMS-ESI (m/z) [M+H]⁺ calcd for C₁₆H₂₅N₂: 245.2012, found 245.2013.

### Step B: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(2R)-2-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from Preparation IX (90 mg, 0.10 mmol) as the appropriate carboxylic acid and the product from Step A (28 mg, 1 eq.) as the appropriate amine, afforded the desired compound (37.5 mg, 37%).

HRMS-ESI (m/z) [M+H]⁺ calcd for C₆₀H₆₈FN₈O₆: 1015.5240, found 1015.5240.

### Example 124: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(3R)-3-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

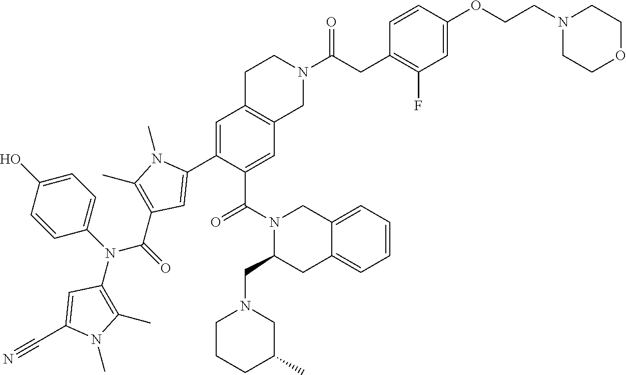 [SMILES: Cc1c(N(C(=O)c2cc(-c3cc4c(cc3C(=O)N3Cc5ccccc5C[C@H]3CN3CCC[C@@H](C)C3)CN(C(=O)Cc3ccc(OCCN5CCOCC5)cc3F)CC4)n(C)c2C)c2ccc(O)cc2)cc(C#N)n1C]

### Step A: (3S)-3-[[(3R)-3-methyl-1-piperidyl]methyl]-1,2,3,4-tetrahydroisoquinoline

(3S)-2-tert-butoxycarbonyl-3,4-dihydro-1H-isoquinoline-3-carboxylic acid (700 mg, 2.5 mmol) and (3R)-3-methylpiperidine hydrochloride (314 mg, 1.4 eq) were reacted using General procedure 5a. Then the crude intermediate was deprotected using General procedure 6c and treated according to General procedure 6a to afford the title product (73 mg, 12%).

¹H NMR (400 MHz, DMSO-d6) δ ppm 7.12-6.98 (m, 4H), 3.91 (s, 2H), 2.95 (m, 1H), 2.82/1.79 (m+m, 2H), 2.71/1.63 (m+m, 2H), 2.65/2.37 (dd+dd, 2H), 2.31/2.27 (dd+dd, 2H), 1.65/0.84 (m+m, 2H), 1.6 (m, 1H), 1.57/1.47 (m+m, 2H), 0.83 (d, 3H); ¹³C NMR (100 MHz, DMSO-d6) δ ppm 64.5, 63.3, 54, 50.9, 48.4, 34.1, 33.2, 31.2, 25.6, 20.1.

HRMS-ESI (m/z) [M+H]⁺ calcd for C₁₆H₂₅N₂: 245.2012, found 245.2013.

### Step B: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(3R)-3-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from Preparation IX (90 mg, 0.10 mmol) as the appropriate carboxylic acid and the product from Step A (28 mg, 1 eq.) as the appropriate amine, afforded the desired compound (50.4 mg, 50%).

HRMS-ESI (m/z) [M+H]⁺ calcd for C₆₀H₆₈FN₈O₆: 1015.5240, found 1015.5236.

### Example 125: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(2S)-2-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

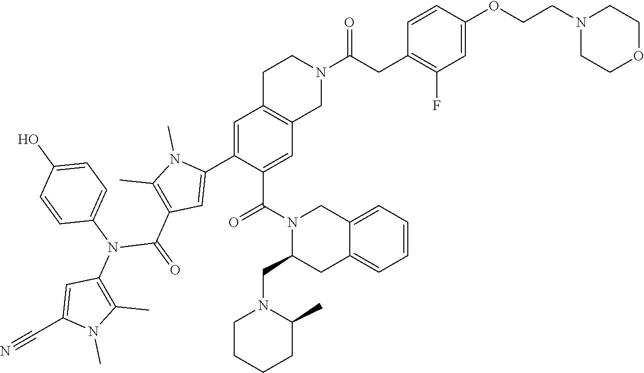 [SMILES: Cc1c(N(C(=O)c2cc(-c3cc4c(cc3C(=O)N3Cc5ccccc5C[C@H]3CN3CCCC[C@@H]3C)CN(C(=O)Cc3ccc(OCCN5CCOCC5)cc3F)CC4)n(C)c2C)c2ccc(O)cc2)cc(C#N)n1C]

### Step A: (3S)-3-[[(2S)-2-methyl-1-piperidyl]methyl]-1,2,3,4-tetrahydroisoquinoline

(3S)-2-tert-butoxycarbonyl-3,4-dihydro-]H-isoquinoline-3-carboxylic acid (700 mg, 2.5 mmol) and (2S)-2-methylpiperidine (350 mg, 1.4 eq) were reacted using General procedure 5a. Then the crude intermediate was deprotected using General procedure 6c and treated according to General procedure 6a to afford the title product (173 mg, 28%).

¹H NMR (400 MHz, DMSO-d6) δ ppm 7.15-7.00 (m, 4H), 3.95 (s, 2H), 2.99 (m, 1H), 2.92/2.05 (m+m, 2H), 2.71/2.16 (dd+dd, 2H), 2.67/2.43 (dd+dd, 2H), 2.28 (m, 1H), 1.68-1.15 (m, 6H), 1.02 (d, 3H); ¹³C NMR (100 MHz, DMSO-d6) δ ppm 58.8, 56.4, 52.9, 51.2, 48.1, 33.6, 19.5.

HRMS-ESI (m/z) [M+H]⁺ calcd for C₁₆H₂₅N₂ 245.2012, found 245.2015.

### Step B: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(2S)-2-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from Preparation IX (90 mg, 0.10 mmol) as the appropriate carboxylic acid and the product from Step A (28 mg, 1 eq.) as the appropriate amine, afforded the desired compound (56.2 mg, 56%).

HRMS-ESI (m/z) [M+H]⁺ calcd for C₆₀H₆₈FN₈O₆: 1015.5240, found 1015.5241.

### Example 126: 5-{7-[(3S)-3-({6-azaspiro[2.5]octan-6-yl}methyl)-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

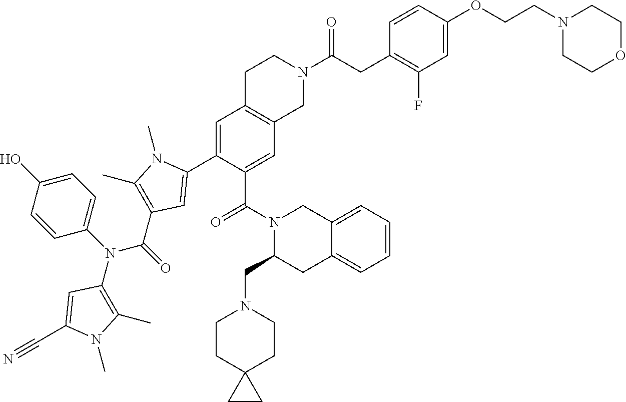 [SMILES: Cc1c(N(C(=O)c2cc(-c3cc4c(cc3C(=O)N3Cc5ccccc5C[C@H]3CN3CCC5(CC3)CC5)CN(C(=O)Cc3ccc(OCCN5CCOCC5)cc3F)CC4)n(C)c2C)c2ccc(O)cc2)cc(C#N)n1C]

### Step A: 6-azaspiro[2.5]octan-6-yl-[(3S)-1,2,3,4-tetrahydroisoquinolin-3-yl]methanone

General procedure 5a, starting from (3S)-2-benzyloxycarbonyl-3,4-dihydro-1H-isoquinoline-3-carboxylic acid (934 mg, 3 mmol) and 6-azaspiro[2.5]octane (400 mg, 1.2 eq.) as reactants, followed with a deprotection step according to General procedure 6b, afforded the title compound (764 mg, 80%).

¹H NMR (400 MHz, DMSO-d6) δ ppm 7.14-6.96 (m, 4H), 3.90 (t, 1H), 3.90 (s, 2H), 3.66-3.45 (m, 4H), 2.80/2.68 (dd+dd, 2H), 2.39 (br., 1H), 1.48-1.22 (m, 4H), 0.34 (s, 4H); ¹³C NMR (100 MHz, DMSO-d6) δ ppm 171.1, 52.8, 47.2, 45.6/42.0, 35.9/34.9, 31.3, 18.3, 11.6.

HRMS-ESI (m/z) [M+H]⁺ calcd for C₁₇H₂₃N₂O: 271.1805, found 271.1806.

### Step B: (3S)-3-(6-azaspiro[2.5]octan-6-ylmethyl)-1,2,3,4-tetrahydroisoquinoline

Using General procedure 6a, starting from the product of Step A (660 mg, 2.44 mmol) the title product was obtained (315 mg, 50%).

¹H NMR (400 MHz, DMSO-d6) δ ppm 7.11-6.97 (m, 4H), 3.91 (s, 2H), 2.95 (m, 1H), 2.66/2.39 (dd+dd, 2H), 2.48/2.38 (m+m, 4H), 2.37/2.32 (dd+dd, 2H), 1.34 (brs, 4H), 0.25 (s, 4H); ¹³C NMR (100 MHz, DMSO-d6) δ ppm 64.4, 54, 50.9, 48.4, 35.2, 34.2, 11.7.

HRMS-ESI (m/z) [M+H]⁺ calcd for C₁₇H₂₅N₂: 257.2012, found 257.2013.

### Step C: 5-{7-[(3S)-3-({6-azaspiro[2.5]octan-6-yl}methyl)-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

Using General procedure 4c, starting from Preparation IX (90 mg, 0.10 mmol) as the appropriate carboxylic acid and the product from Step B (30 mg, 1 eq.) as the appropriate amine, afforded the desired product (59.8 mg, 58%).

HRMS-ESI (m/z) [M+H]⁺ calcd for C₆₁H₆₈FN₈O₆: 1027.5240, found 1027.5235.

### Example 127: N-(5-cyano-1,2-dimethyl-1H-pyrrol-3-yl)-N-(4-hydroxyphenyl)-1,2-dimethyl-5-[2-(2-{4-[(2S or R)-2-(morpholin-4-yl)propoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide (Diastereomer 1)

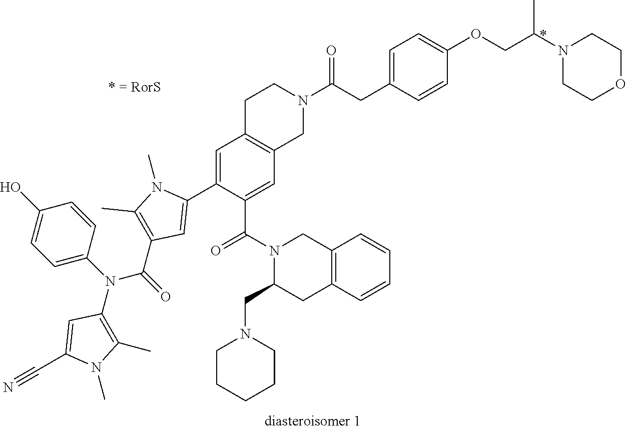 [SMILES: Cc1c(N(C(=O)c2cc(-c3cc4c(cc3C(=O)N3Cc5ccccc5C[C@H]3CN3CCCCC3)CN(C(=O)Cc3ccc(OCC(C)N5CCOCC5)cc3)CC4)n(C)c2C)c2ccc(O)cc2)cc(C#N)n1C]

### Step A: methyl 2-[4-((2S)-2-morpholinopropoxy)phenyl]acetate and methyl 2-[4-((2R)-2-morpholinopropoxy)phenyl]acetate

Using the General procedure 7b, starting from methyl 2-(4-hydroxyphenyl)acetate (1.40 g, 8.4 mmol) as the appropriate phenol and 1-morpholinopropan-2-ol (1.10 g, 1.1 eq.) as the appropriate alcohol, the mixture of enantiomers were obtained, which were separated by HILIC chromatography (using ACN and 5 mM aq. NH₄HCO₃ solution as eluent) to give methyl-2-[4-(1-methyl-2-morpholino-ethoxy)phenyl]acetic acetate (560 mg, 25%) and methyl 2-[4-(2-morpholinopropoxy)phenyl]acetate (660 mg, 30%).

¹H NMR (500 MHz, DMSO-d6) δ ppm 7.16 (m, 2H), 6.89 (m, 2H), 4.05/3.87 (dd+dd, 2H), 3.62 (s, 3H), 3.58 (s, 2H), 3.57 (m, 4H), 2.89 (m, 1H), 2.57 (m, 4H), 1.09 (d, 3H); ¹³C NMR (125 MHz, DMSO-d6) δ ppm 172.2, 130.7, 115.2, 70.4, 67.2, 58.4, 51.9, 49.9, 39.9, 13.3.

HRMS-ESI (m/z) [M+H]⁺ calcd for C₁₆H₂₄NO₄: 294.1699, found 294.1702.

Enantiomers of methyl 2-[4-(2-morpholinopropoxy)phenyl]acetate was separated on a Daicel IC chiral column (50*500 mm, 20 m) using 2:8:90 CH₂Cl₂/2-PrOH/Heptane+0.1% DEA as eluents to give the Enantiomer 1 (99.9% ee) and Enantiomer 2 (99.0% ee).

### Step B: 2-[4-((2S or R)-2-morpholinopropoxy)phenyl]acetic acid (from Enantiomer 1)

Using the General procedure 8, starting from the Enantiomer 1 of Step A (325 mg, 1.11 mmol), the desired product was obtained (349 mg, quant.).

¹H NMR (500 MHz, DMSO-d6) δ ppm 11.81 (br., 1H), 7.20 (dm, 2H), 6.95 (dm, 2H), 4.34 (d, 2H), 4.04-3.90 (m, 4H), 3.73 (m, 1H), 3.50 (s, 2H), 3.40/3.32-3.15 (d+m, 4H), 1.44 (d, 3H); ¹³C NMR (125 MHz, DMSO-d6) δ ppm 173.3, 156.8, 130.9, 128.4, 115.0, 66.9, 63.6, 60.4, 49.6/49.1, 40.2, 12.0.

HRMS-ESI (m/z) [M+H]⁺ calcd for C₁₅H₂₂NO₄: 280.1544, found 280.1544.

### Step C: N-(5-cyano-1,2-dimethyl-1H-pyrrol-3-yl)-N-(4-hydroxyphenyl)-1,2-dimethyl-5-[2-(2-{4-[(2S or R)-2-(morpholin-4-yl)propoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide (Diastereomer 1)

Using General procedure 4c, starting from the product of Step B (160.0 mg, 4.8 eq.) as the appropriate carboxylic acid and Preparation VIIa (90 mg, 0.11 mmol) as the appropriate amine, afforded the title compound (69.0 mg, 65%).

HRMS-ESI (m/z) [M+H]⁺ calcd for C₆₀H₆₉N₈O₆: 997.5340, found 997.5333.

### Example 128: N-(5-cyano-1,2-dimethyl-1H-pyrrol-3-yl)-N-(4-hydroxyphenyl)-1,2-dimethyl-5-[2-(2-{4-[(2R or S)-2-(morpholin-4-yl)propoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide (Diastereomer 2)

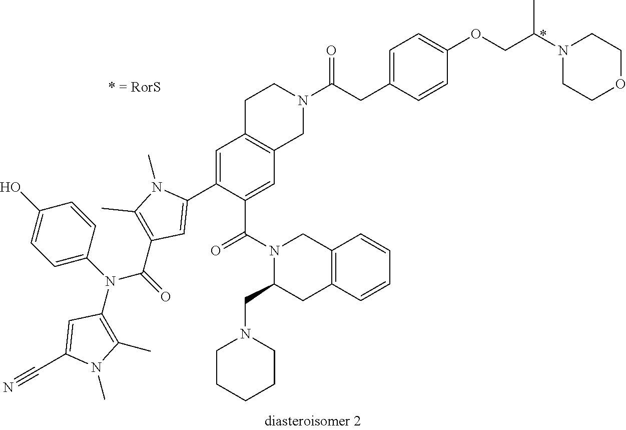 [SMILES: Cc1c(N(C(=O)c2cc(-c3cc4c(cc3C(=O)N3Cc5ccccc5C[C@H]3CN3CCCCC3)CN(C(=O)Cc3ccc(OCC(C)N5CCOCC5)cc3)CC4)n(C)c2C)c2ccc(O)cc2)cc(C#N)n1C]

### Step A: 2-[4-((2R or S)-2-morpholinopropoxy)phenyl]acetic acid (from Enantiomer 2)

Using the General procedure 8, starting from the Enantiomer 2 of Step A of Example 127 (328 mg, 1.12 mmol), the desired product was obtained (353 mg, quant.).

¹H NMR (500 MHz, DMSO-d6) δ ppm 11.81 (br., 1H), 7.20 (dm, 2H), 6.95 (dm, 2H), 4.34 (d, 2H), 4.04-3.90 (m, 4H), 3.73 (m, 1H), 3.50 (s, 2H), 3.40/3.32-3.15 (d+m, 4H), 1.44 (d, 3H); ¹³C NMR (125 MHz, DMSO-d6) δ ppm 173.3, 156.8, 130.9, 128.4, 115.0, 66.9, 63.6, 60.4, 49.6/49.1, 40.2, 12.0.

HRMS-ESI (m/z) [M+H]⁺ calcd for C₁₅H₂₂NO₄: 280.1544, found 280.1544.

### Step B: N-(5-cyano-1,2-dimethyl-1H-pyrrol-3-yl)-N-(4-hydroxyphenyl)-1,2-dimethyl-5-[2-(2-{4-[(2R or S)-2-(morpholin-4-yl)propoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide (Diastereomer 2)

Using General procedure 4c, starting from the product of Step A (200 mg, 6.0 eq.) as the appropriate carboxylic acid and Preparation VIIa (90 mg, 0.11 mmol) as the appropriate amine, afforded the title compound (69 mg, 65%).

HRMS-ESI (m/z) [M+H]⁺ calcd for C₆₀H₆₉N₈O₆: 997.5340, found 997.5333.

### Example 129: N-[5-cyano-1-(difluoromethyl)-2-methylpyrrol-3-yl]-5-{2-[2-(4-{2-[cis-2,6-dimethylmorpholin-4-yl]ethoxy}phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

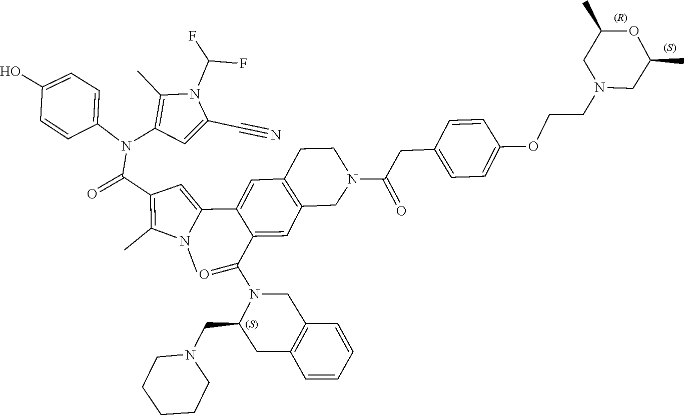

Using General procedure 4a and the product from Example 77, Step C (143 mg, 0.15 mmol, 1 eq.) as the appropriate amine and the product from Example 39, Step B (50 mg, 0.17 mmol, 1.1 eq.) as the appropriate carboxylic acid afforded an intermediate which was treated according to General procedure 3b with heating at 50° C. to afford the title product (55.9 mg, 0.05 mmol, 36%).

HRMS calcd for C₆₁H₆₈F₂N₈O₆: 1046.5230, found 1047.5304 [M+H]⁺

### Example 130: N-(5-cyano-1,2-dimethyl-1H-pyrrol-3-yl)-5-{7-[(3S)-3-[(4,4-difluoropiperidin-1-yl)methyl]-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(4-hydroxyphenyl)-1,2-dimethyl-1H-pyrrole-3-carboxamide

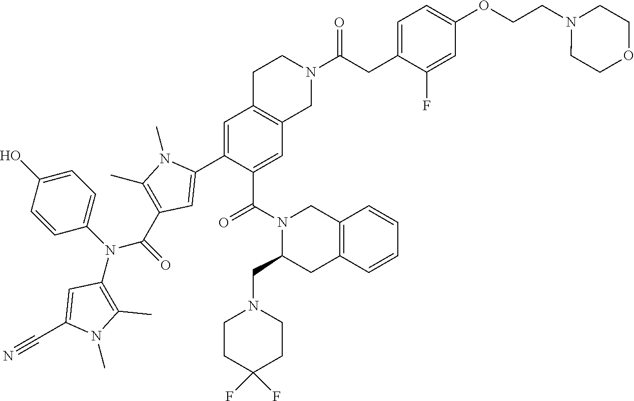 [SMILES: Cc1c(N(C(=O)c2cc(-c3cc4c(cc3C(=O)N3Cc5ccccc5C[C@H]3CN3CCC(F)(F)CC3)CN(C(=O)Cc3ccc(OCCN5CCOCC5)cc3F)CC4)n(C)c2C)c2ccc(O)cc2)cc(C#N)n1C]

### Step A: (4,4-difluoro-1-piperidyl)-[(3S)-1,2,3,4-tetrahydroisoquinolin-3-yl]methanone

Using the General procedure 5a, starting from (3S)-2-benzyloxycarbonyl-3,4-dihydro-]H-isoquinoline-3-carboxylic acid (934 mg, 3.00 mmol) as the appropriate carboxylic acid and 4,4-difluoropiperidine hydrochloride (567 mg, 1.2 eq) as the appropriate amine, followed with a deprotection step according to General procedure 6b, afforded the title compound (705 mg, 84%).

¹H NMR (400 MHz, DMSO-d6) δ ppm 7.12 (m, 1H), 7.11 (m, 1H), 7.10 (m, 1H), 7.02 (m, 1H), 3.94 (dd, 1H), 3.93/3.88 (d+d, 2H), 3.74/3.67/3.54/3.54 (m+m/m+m, 4H), 2.87/2.70 (dd+dd, 2H), 2.06/1.95 (m/m, 4H); ¹³C NMR (100 MHz, DMSO-d6) δ ppm 129.7, 126.2, 126.1, 126.0, 50.0, 47.3, 42.3/38.9, 34.8/33.7, 30.9.

HRMS-ESI (m/z) [M+H]⁺ calcd for C₁₅H₁₉F₂N₂O: 281.1460, found 281.1463.

### Step B: (3S)-3-[(4,4-difluoro-1-piperidyl)methyl]-1,2,3,4-tetrahydroisoquinoline

Using General procedure 6a, starting from the product of Step A (700 mg, 2.50 mmol), afforded the title compound (552 mg, 83%).

¹H NMR (400 MHz, DMSO-d6) δ ppm 7.17-6.94 (m, 4H), 3.90 (s, 2H), 2.93 (m, 1H), 2.67/2.39 (dd+m, 2H), 2.57/2.50 (m+m, 4H), 2.40 (m, 2H), 1.97 (m, 4H); ¹³C NMR (100 MHz, DMSO-d6) δ ppm 123.4, 62.7, 51.1, 50.6, 48.3, 34.0, 34.0.

HRMS-ESI (m/z) [M+H]⁺ calcd for C₁₅H₂₁F₂N₂: 267.1667, found 267.1669.

### Step C. N-(5-cyano-1,2-dimethyl-1H-pyrrol-3-yl)-5-{7-[(3S)-3-[(4,4-difluoropiperidin-1-yl)methyl]-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(4-hydroxyphenyl)-1,2-dimethyl-1H-pyrrole-3-carboxamide

Using General procedure 4c, starting from Preparation IX (80 mg, 0.09 mmol) as the appropriate carboxylic acid and the product of Step B (35 mg, 1.5 eq.) as the appropriate amine, afforded the title compound (40 mg, 43%).

HRMS-ESI (m/z) [M+H]⁺ calcd for C₅₉H₆₄F₃N₈O₆: 1037.4895, found 1037.4894.

### Example 131: N-(5-cyano-1,2-dimethylpyrrol-3-yl)-5-[2-(2-{4-[2-(4,4-difluoropiperidin-1-yl)ethyl]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

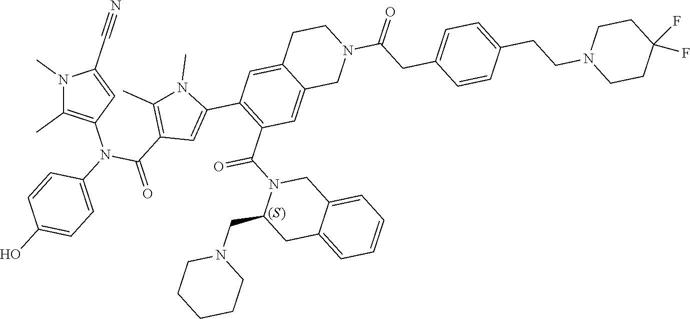

Using General procedure 4a and Preparation IVb (125 mg, 0.17 mmol, 1 eq.) as the appropriate amine and the product from Example 51, Step B (53 mg, 0.19 mmol, 1.1 eq.) as the appropriate carboxylic acid afforded the title product (25.4 mg, 0.03 mmol, 15%).

HRMS calcd for C₆₀H₆₆F₂N₈O₄: 1000.5175, found 1001.5248 [M+H]⁺

### Example 132: N-(5-cyano-1,2-dimethyl-1H-pyrrol-3-yl)-N-(4-hydroxyphenyl)-1,2-dimethyl-5-{2-[2-(4-{[2-methyl-1-(morpholin-4-yl)propan-2-yl]oxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-1H-pyrrole-3-carboxamide

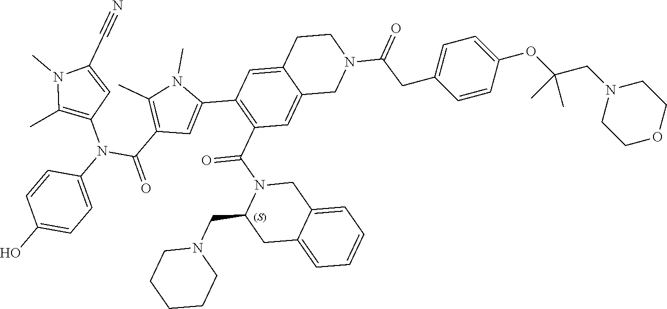

### Step A: methyl 2-(4-{[2-methyl-1-(morpholin-4-yl)propan-2-yl]oxy]phenyl)acetate

Using General procedure 7b and methyl 4-hydroxyphenylacetate (500 mg, 3.01 mmol, 1 eq.) as the appropriate phenol and 2-methyl-1-(morpholin-4-yl)propan-2-ol (719 mg, 4.51 mmol, 1.5 eq.) as the appropriate alcohol afforded the title product (0.92 g, 3.0 mmol, quant.).

LRMS calcd for C₁₇H₂₅NO₄: 307.2, found 308.4 [M+H]⁺

¹H NMR (400 MHz, DMSO-d6) δ ppm: 7.18-7.12 (m, 2H), 6.93-6.88 (m, 2H), 3.62-3.55 (m, 9H), 2.58-2.51 (m, 4H), 2.47 (s, 2H), 1.22 (s, 6H).

### Step B: 2-(4-{[2-methyl-1-(morpholin-4-yl)propan-2-yl]oxy}phenyl)acetic acid

Using General procedure 8 and the product from Step A (920 mg, 2.99 mmol, 1 eq.) as the appropriate ester afforded the title product (862.3 mg, 2.94 mmol, 98%).

LRMS calcd for C₁₆H₂₃NO₄: 293.2, found 294.0 [M+H]⁺

¹H NMR (400 MHz, DMSO-d6) δ ppm: 12.34 (br s, 1H), 7.27-6.76 (br m, 4H), 4.32-2.14 (br m, 12H), 1.30 (br s, 6H).

### Step C: N-(5-cyano-1,2-dimethyl-1H-pyrrol-3-yl)-N-(4-hydroxyphenyl)-1,2-dimethyl-5-{2-[2-(4-{[2-methyl-1-(morpholin-4-yl)propan-2-yl]oxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-1H-pyrrole-3-carboxamide

Using General procedure 4a and Preparation IVb (125 mg, 0.17 mmol, 1 eq.) as the appropriate amine and the product from Step B (55 mg, 0.19 mmol, 1.1 eq.) as the appropriate carboxylic acid afforded the title product (49.6 mg, 0.05 mmol, 29%).

HRMS calcd for C₆₁H₇₀N₈O₆: 1010.5418, found 1011.5493 [M+H]⁺

### Example 133: N-(5-cyano-1,2-dimethyl-1H-pyrrol-3-yl)-N-(4-hydroxyphenyl)-1,2-dimethyl-5-[2-(2-phenoxyacetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

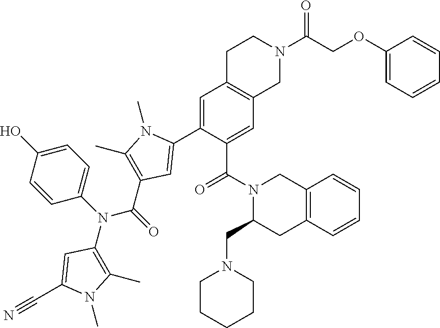 [SMILES: Cc1c(N(C(=O)c2cc(-c3cc4c(cc3C(=O)N3Cc5ccccc5C[C@H]3CN3CCCCC3)CN(C(=O)COc3ccccc3)CC4)n(C)c2C)c2ccc(O)cc2)cc(C#N)n1C]

Using General procedure 4c, starting from 2-phenoxyacetic acid (150 mg, 9.5 eq.) as the appropriate carboxylic acid and Preparation VIIa (88 mg, 0.10 mmol) as the appropriate amine afforded the title compound (52 mg, 58%).

HRMS-ESI (m/z) [M+H]⁺ calcd for C₅₃H₅₅N₇O₅: 870.4337, found 870.4336.

### Example 134: N-(5-cyano-1,2-dimethyl-1H-pyrrol-3-yl)-5-{2-[2-(2-fluoro-4-{2-[(2-fluoroethyl)(methyl)amino]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

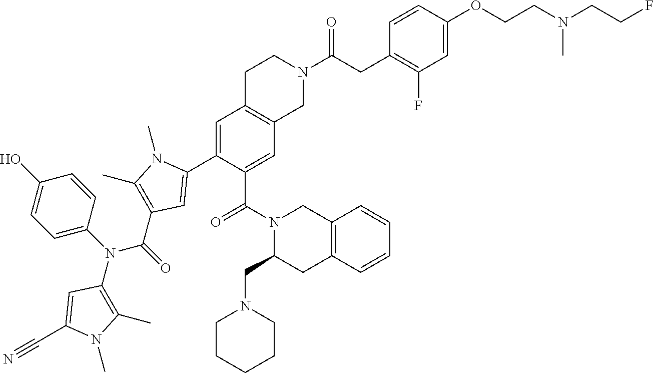 [SMILES: Cc1c(N(C(=O)c2cc(-c3cc4c(cc3C(=O)N3Cc5ccccc5C[C@H]3CN3CCCCC3)CN(C(=O)Cc3ccc(OCCN(C)CCF)cc3F)CC4)n(C)c2C)c2ccc(O)cc2)cc(C#N)n1C]

### Step A: 2-[2-fluoro-4-[2-[2-fluoroethyl(methyl)amino]ethoxy]phenyl]acetic acid

Using the General procedure 7d and then General procedure 8, starting from Preparation X (250 mg, 0.86 mmol) as the appropriate alkyl halide and 2-fluoro-N-methyl-ethanamine, hydrogen chloride (1:1) (200 mg, 2.0 eq.) as the appropriate amine, the desired product was obtained (162 mg, 69%).

¹H NMR (500 MHz, DMSO-d6) δ ppm 7.20 (t, 1H), 6.80 (dd, 1H), 6.73 (dd, 1H), 4.51 (dt, 2H), 4.05 (t, 2H), 3.51 (s, 2H), 2.78 (t, 2H), 2.74 (dt, 2H), 2.30 (s, 3H); ¹³C NMR (125 MHz, DMSO-d6) δ ppm 172.5, 161.6, 159.3, 132.7, 114.5, 110.9, 102.2, 82.7, 66.7, 57.4, 56.3, 43.1, 34.0.

HRMS-ESI (m/z) [M+H]⁺ calcd for C₁₃H₁₈F₂NO₃: 274.1249, found 274.1251.

### Step B: N-(5-cyano-1,2-dimethyl-1H-pyrrol-3-yl)-5-{2-[2-(2-fluoro-4-{2-[(2-fluoroethyl)(methyl)amino]ethoxy}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide

Using General procedure 4c, starting from the product of Step A (80 mg, 2.8 eq.) as the appropriate carboxylic acid and Preparation VIIa (90 mg, 0.11 mmol) as the appropriate amine, afforded the title compound (59 mg, 56%).

HRMS-ESI (m/z) [M+H]⁺ calcd for C₅₈H₆₅F₂N₈O₅: 991.5046, found 991.5036.

### Example 135: N-(5-cyano-1,2-dimethyl-1H-pyrrol-3-yl)-5-[2-(2-{2-fluoro-4-[(2S or R)-2-(morpholin-4-yl)propoxyphenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 1)

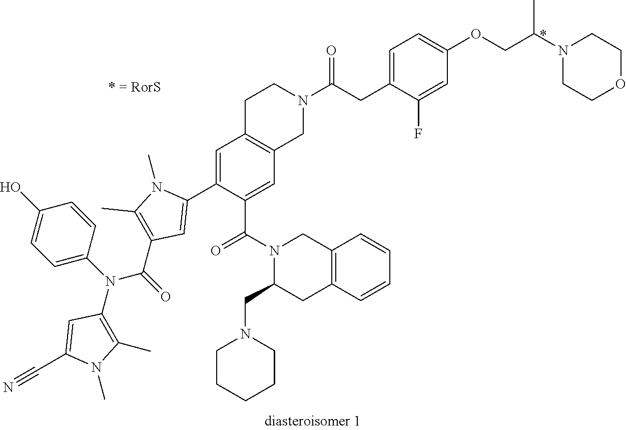 [SMILES: Cc1c(N(C(=O)c2cc(-c3cc4c(cc3C(=O)N3Cc5ccccc5C[C@H]3CN3CCCCC3)CN(C(=O)Cc3ccc(OCC(C)N5CCOCC5)cc3F)CC4)n(C)c2C)c2ccc(O)cc2)cc(C#N)n1C]

### Step A: methyl 2-[2-fluoro-4-((2S)-2-morpholinopropoxy)phenyl]acetate and methyl 2-[2-fluoro-4-((2R)-2-morpholinopropoxy)phenyl]acetate

Using the General procedure 7b, starting from methyl 2-(2-fluoro-4-hydroxy-phenyl)acetate (1.0 g, 5.43 mmol) as the appropriate phenol and 1-morpholinopropan-2-ol (1.02 g, 1.3 eq.) as the appropriate alcohol, the mixture of enantiomers were obtained, which were separated by HILIC chromatography (using ACN:NH₄HCO₃ as eluent) to give methyl 2-[2-fluoro-4-(1-methyl-2-morpholino-ethoxy)phenyl]acetate (570 mg, 34%) and methyl 2-[2-fluoro-4-(2-morpholinopropoxy)phenyl]acetate (820 mg, 48%).

¹H NMR (400 MHz, DMSO-d6) δ ppm 7.22 (t, 1H), 6.83 (dd, 1H), 6.75 (dd, 1H), 4.05/3.86 (dd+dd, 2H), 3.63 (s, 2H), 3.61 (s, 3H), 3.55 (t, 4H), 2.89 (m, 1H), 2.55 (m, 4H), 1.06 (d, 3H); ¹³C NMR (100 MHz, DMSO-d6) δ ppm 171.5, 132.7, 111.2, 102.4, 70.3, 67.1, 58.2, 52.3, 49.7, 33.6, 13.2.

HRMS-ESI (m/z) [M+H]⁺ calcd for C₁₆H₂₃FNO₄: 312.1605, found 312.1607.

Enantiomers of methyl 2-[2-fluoro-4-(2-morpholinopropoxy)phenyl]acetate was separated on an OJ chiral column (250*4.6 mm, 5 m) using 20:80 2-PrOH/HEPTANE+0.1% DEA as eluents to give the Enantiomer 1 (99.9% ee) and Enantiomer 2 (97.0% ee).

### Step B: 2-[2-fluoro-4-((2S or R)-2-morpholinopropoxy)phenyl]acetic acid (from Enantiomer 1)

Using the General procedure 8, starting from the Enantiomer 1 of Step A (400 mg, 1.28 mmol), the desired product was obtained (300 mg, 79%).

¹H NMR (400 MHz, DMSO-d6) δ ppm 12.41 (br., 1H), 7.20 (t, 1H), 6.81 (dd, 1H), 6.74 (dd, 1H), 4.05/3.86 (dd+dd, 2H), 3.55 (t, 4H), 3.51 (s, 2H), 2.86 (m, 1H), 2.55 (m, 4H), 1.06 (d, 3H); ¹³C NMR (100 MHz, DMSO-d6) δ ppm 172.5, 161.5, 159.4, 132.6, 114.6, 111.0, 102.3, 70.3, 67.1, 58.2, 49.7, 34.0, 13.2.

HRMS-ESI (m/z) [M+H]⁺ calcd for C₁₅H₂₁FNO₄: 298.1449, found 298.1450.

### Step C: N-(5-cyano-1,2-dimethyl-1H-pyrrol-3-yl)-5-[2-(2-{2-fluoro-4-[(2S or R)-2-(morpholin-4-yl)propoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 1)

Using General procedure 4c, starting from the product of Step B (60.0 mg, 2.1 eq.) as the appropriate carboxylic acid and Preparation VIIa (80 mg, 0.09 mmol) as the appropriate amine, afforded the title compound (65.0 mg, 68%).

HRMS-ESI (m/z) [M+H]⁺ calcd for C₆₀H₆₈FN₈O₆: 1015.5246, found 1015.5230.

### Example 136: N-(5-cyano-1,2-dimethyl-pyrrol-3-yl)-3-[2-[2-[2-fluoro-4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)indolizine-1-carboxamide

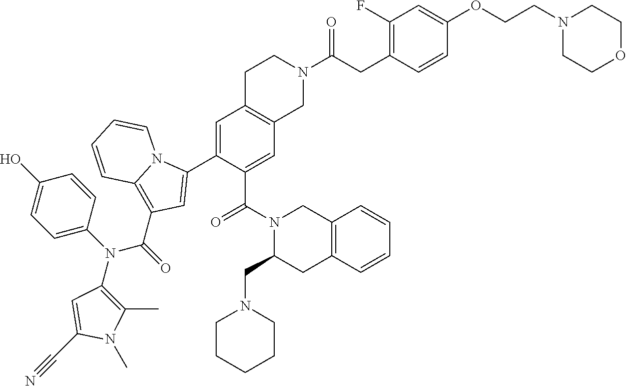 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCOCC5)cc3F)CC4)n3ccccc23)c2ccc(O)cc2)cc(C#N)n1C]

### Step A: methyl indolizine-1-carboxylate

To indolizine-1-carboxylic acid (1.7 g, 10.7 mmol) in DCM (160 mL) was added 1-chloro-N,N,2-trimethyl-prop-1-en-1-amine (1.7 g, 10.7 mmol). After 30 min, the reaction mixture was treated with MeOH (8.6 mL, 20 eq.) and stirred until complete conversion. The mixture was concentrated and the residue was purified by flash chromatography using heptane and EtOAc as eluents to give the title compound (1.6 g, 84%).

¹H NMR (500 MHz, DMSO-d6) δ ppm 8.48 (dm, 1H), 8.03 (dm, 1H), 7.60 (dd, 1H), 7.16 (ddd, 1H), 7.14 (d, 1H), 6.85 (td, 1H), 3.78 (s, 3H); ¹³C NMR (125 MHz, DMSO-d6) δ ppm 164.7, 135.4, 127.8, 123.5, 119, 115.8, 115.3, 112.9, 102.7, 51.1.

HRMS-ESI (m/z) [M+H]⁺ calcd for C₁₀H₁₀NO₂: 176.0706, found 176.0706.

### Step B: benzyl 7-formyl-6-(1-methoxycarbonylindolizin-3-yl)-3,4-dihydro-1H-isoquinoline-2-carboxylate

To benzyl 6-bromo-7-formyl-3,4-dihydro-]H-isoquinoline-2-carboxylate (2.4 g, 6.6 mmol) and the product of Step A (1.7 g, 1.5 eq.) in DMF (8.5 mL) was added K₂CO₃ (439 mg, 2.0 eq.) and diacetoxypalladium (147 mg, 0.1 eq.) and the mixture was stirred at 100° C. until complete conversion was observed. The reaction mixture was concentrated and purified by flash chromatography using heptane and EtOAc as eluents, to give the title compound (2.1 g, 70%).

¹H NMR (500 MHz, DMSO-d6) δ ppm 9.74 (s, 1H), 8.17 (dm, 1H), 8.04 (d, 1H), 7.91 (s, 1H), 7.49 (s, 1H), 7.46-7.30 (m, 5H), 7.26 (ddd, 1H), 7.24 (s, 1H), 6.86 (td, 1H), 5.16 (s, 2H), 4.80/4.74 (br/br., 2H), 3.81 (s, 3H), 3.71 (br., 2H), 2.96 (t, 2H); ¹³C NMR (125 MHz, DMSO-d6) δ ppm 191.6, 164.6, 155.1, 132.8, 128.8, 125.2, 124.1, 119.3, 118.3, 113.8, 66.9, 51.2, 45.6, 41.3/41.0, 29.0/28.8.

HRMS-ESI (m/z) [M+H]⁺ calcd for C₂₈H₂₅N₂O₅: 469.1758, found 469.1756.

### Step C: 2-benzyloxycarbonyl-6-(1-methoxycarbonylindolizin-3-yl)-3,4-dihydro-1H-isoquinoline-7-carboxylic acid

To the product of Step B (2.1 g, 4.48 mmol) and 2-methylbut-2-ene (3.6 mL, 7.5 eq.) in 2-methylpropan-2-ol (21 mL) and THE (14 mL) was added dropwise NaH₂PO₄ (2.7 g, 5.0 eq.) in water (18 mL). After cooling to 14° C., the mixture was treated with NaClO₂ (1.2 g, 3.0 eq.) in water (18 mL) dropwise, the mixture was stirred at 20° C. until complete conversion was observed. After the reaction mixture was cooled to 5-10° C., treated dropwise with Na₂S₂O₃ (945 mg, 1.3 eq.) in water (14 mL), and diluted with MTBE (100 mL), the organic phase was washed with brine (100 mL), dried, concentrated, and purified by flash chromatography using heptane and EtOAc as eluents, to give the title compound (1.6 g, 74%).

¹H NMR (500 MHz, DMSO-d6) δ ppm 12.73 (brs, 1H), 8.11 (d, 1H), 7.89 (s, 1H), 7.80 (d, 1H), 7.43-7.31 (m, 5H), 7.33 (s, 1H), 7.19 (dd, 1H), 7.06 (s, 1H), 6.80 (t, 1H), 5.15 (s, 2H), 4.76/4.70 (brs/brs, 2H), 3.80 (s, 3H), 3.70 (brt, 2H), 2.90 (t, 2H); ¹³C NMR (125 MHz, DMSO-d6) δ ppm 167.7, 164.8, 155.1, 139.7, 137.3, 135.6, 133.6, 130.3, 129.1, 128.7, 126.0, 125.9, 125.0, 123.3, 119.1, 115.6, 113.0, 102.7, 66.9, 51.1, 45.6/45.5, 41.4/41.1, 28.6/28.4.

HRMS-ESI (m/z) [M+H]⁺ calcd for C₂₈H₂₅N₂O₆: 485.1710, found 485.1708.

### Step D: 3-[7-[(3S)-3-(1-piperidylmethyl)-3,4-dihydro-1H-isoquinoline-2-carbonyl]-1,2,3,4-tetrahydroisoquinolin-6-yl]indolizine-1-carboxylic acid

Using the procedure described at Step A of Preparation VIIa, starting from the product of Step C (322 mg, 0.66 mmol) as the appropriate carboxylic acid and (3S)-3-(1-piperidylmethyl)-1,2,3,4-tetrahydroisoquinoline, hydrogen chloride (1:1) (213 mg, 1.2 eq.) as the appropriate amine, the title compound was obtained (400 mg, 88%).

¹H NMR (500 MHz, DMSO-d6) δ ppm 11.76 (brs, 1H), 9.95 (brs, 1H), 7.76-6.50 (m, 11H), 5.32-1.28 (m, 23H).

HRMS-ESI (m/z) [M+H]⁺ calcd for C₃₄H₃₇N₄O₃: 549.2860, found 549.2862.

### Step E: 3-[2-(9H-fluoren-9-ylmethoxycarbonyl)-7-[(3S)-3-(1-piperidylmethyl)-3,4-dihydro-1H-isoquinoline-2-carbonyl]-3,4-dihydro-1H-isoquinolin-6-yl]indolizine-1-carboxylic acid

To a mixture of the product of Step D (400 mg, 0.73 mmol.) and NaHCO₃ (141 mg, 2.3 eq.) in 1,4-dioxane (4 mL) and water (4 mL) was added 9H-fluoren-9-ylmethyl carbonochloridate (208 mg, 1.1 eq.). The reaction mixture was stirred until complete conversion was observed.

After the addition of a 2 M solution of HCl (7 mL) and stirring for 15 min, the reaction mixture was diluted with DCM (50 mL). The organic phase was washed with brine (50 ml), dried, concentrated, and purified by flash chromatography using DCM and MeOH as eluents, to give the title compound (240 mg, 43%).

¹H NMR (400 MHz, DMSO-d6) δ ppm 11.82 (brs, 1H), 9.93 (brs, 1H), 8.23-6.52 (m, 19H), 5.40-1.28 (m, 26H).

HRMS-ESI (m/z) [M+H]⁺ calcd for C₄₉H₄₇N₄O₅: 771.3541, found 771.3543.

### Step F. 9H-fluoren-9-ylmethyl 6-[1-[[4-[tert-butyl(dimethyl)silyl]oxyphenyl]-(5-cyano-1,2-dimethyl-pyrrol-3-yl)carbamoyl]indolizin-3-yl]-7-[(3S)-3-(1-piperidylmethyl)-3,4-dihydro-1H-isoquinoline-2-carbonyl]-3,4-dihydro-1H-isoquinoline-2-carboxylate

Using General procedure 2b, starting from the product of Step E (240 mg, 0.31 mmol) as the appropriate acid and the product from Step B of Preparation IVa (128 mg, 1.2 eq.) as the appropriate aniline, the title compound was obtained (233 mg, 64%).

¹H NMR (400 MHz, DMSO-d6) δ ppm 8.23-6.51 (m, 23H), 5.86-5.60 (s, 1H), 5.01-1.08 (m, 26H), 3.65-3.53 (s, 3H), 2.08-1.82 (s, 3H), 0.87/0.86 (s/s, 9H), 0.14-0.09 (s, 6H).

HRMS-ESI (m/z) [M+H]⁺ calcd for C₆₈H₇₂N₇O₅Si: 1094.5351, found 1094.5353.

### Step G: N-[4-[tert-butyl(dimethyl)silyl]oxyphenyl]-N-(5-cyano-1,2-dimethyl-pyrrol-3-yl)-3-[7-[(3S)-3-(1-piperidylmethyl)-3,4-dihydro-1H-isoquinoline-2-carbonyl]-1,2,3,4-tetrahydroisoquinolin-6-yl]indolizine-1-carboxamide

The product of Step F (230 mg, 0.20 mmol) in morpholine (1 mL) and DCM (1 mL) was stirred until complete conversion was observed. The reaction mixture was concentrated and purified by flash chromatography using DCM and MeOH as eluents, to give the title compound (145 mg, 85%).

¹H NMR (400 MHz, DMSO-d6) δ ppm 8.21-6.48 (m, 15H), 5.76/5.62 (s, 1H), 4.96-1.22 (m, 24H), 3.65-3.55 (s, 3H), 2.04/1.84 (s, 3H), 0.89 (s, 9H), 0.15-0.11 (s, 6H).

HRMS-ESI (m/z) [M+H]⁺ calcd for C₅₃H₆₂N₇O₃Si: 872.4670, found 872.4671.

### Step H: N-(5-cyano-1,2-dimethyl-pyrrol-3-yl)-3-[2-[2-[2-fluoro-4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)indolizine-1-carboxamide

Using General procedure 4c, starting from the product of Preparation Vb (40 mg, 1.5 eq.) as the appropriate carboxylic acid and the product of Step G (80 mg, 0.09 mmol) as the appropriate amine, the title compound was obtained (41 mg, 44%).

HRMS-ESI (m/z) [M+H]⁺ calcd for C₆₁H₆₄FN₈O₆: 1023.4922, found 1023.4924.

### Example 137: N-(5-cyano-1,2-dimethyl-1H-pyrrol-3-yl)-5-{7-[(3S)-3-[(diethylamino)methyl]-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(4-hydroxyphenyl)-1,2-dimethyl-1H-pyrrole-3-carboxamide

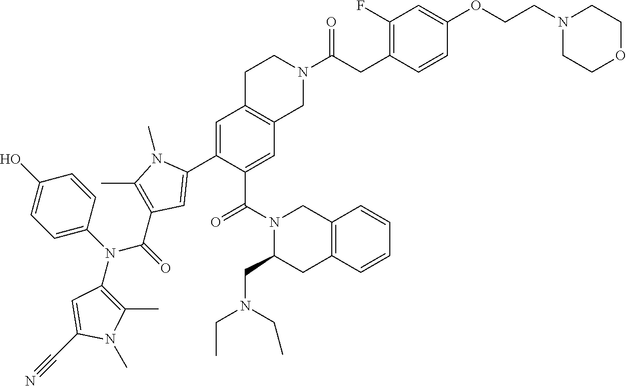 [SMILES: CCN(CC)C[C@@H]1Cc2ccccc2CN1C(=O)c1cc2c(cc1-c1cc(C(=O)N(c3ccc(O)cc3)c3cc(C#N)n(C)c3C)c(C)n1C)CCN(C(=O)Cc1ccc(OCCN3CCOCC3)cc1F)C2]

### Step A: tert-butyl (3S)-3-(diethylcarbamoyl)-3,4-dihydro-1H-isoquinoline-2-carboxylate

Using the General procedure 5a, starting from (3S)-2-tert-butoxycarbonyl-3,4-dihydro-]H-isoquinoline-3-carboxylic acid (850 mg, 3.06 mmol, 1.0 eq.) as the appropriate carboxylic acid and diethylamine (448 mg, 6.13 mmol, 2.0 eq.) as the appropriate amine, afforded the title product (940 mg, 85%).

¹H NMR (400 MHz, DMSO-d6) δ ppm 7.27-7.05 (m, 4H), 5.05/4.77 (m, 1H), 4.7/4.68/4.37/4.26 (d+d, 2H), 3.58-2.96 (m, 4H), 3.11/3.07/2.87/2.81 (dd+dd, 2H), 1.42/1.36 (s, 9H), 1.24-0.88 (t, 6H).

HRMS-ESI (m/z) [M+H]⁺ calcd for C₁₉H₂₉N₂O₃: 333.2173, found 333.2172.

### Step B: N-ethyl-N-[[(3S)-1,2,3,4-tetrahydroisoquinolin-3-yl]methyl]ethanamine

To a solution of the product of Step A (940 mg, 2.60 mmol 1.0 eq.) in ACN (26 mL) was added HCl (12 M aq. solution, 1.0 mL, 5.0 eq.). The reaction mixture was stirred until the deprotection was complete and concentrated. The mixture was diluted with toluene and concentrated. The residue was treated according to General procedure 6a, to give the title compound (496 mg, 83%).

¹H NMR (500 MHz, DMSO-d6) δ ppm 7.13-6.96 (m, 4H), 3.91/3.90 (d+d, 2H), 2.83 (m, 1H), 2.66/2.36 (dd+dd, 2H), 2.52/2.47 (m+m, 4H), 2.40/2.37 (m+m, 2H), 0.97 (t, 6H); ¹³C NMR (125 MHz, DMSO-d6) δ ppm 59.1, 52.0, 48.5, 47.6, 34.2, 12.4.

HRMS-ESI (m/z) [M+H]⁺ calcd for C₁₄H₂₃N₂: 219.1856, found 219.1855.

### Step C. N-(5-cyano-1,2-dimethyl-1H-pyrrol-3-yl)-5-{7-[(3S)-3-[(diethylamino)methyl]-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(4-hydroxyphenyl)-1,2-dimethyl-1H-pyrrole-3-carboxamide

Using General procedure 4c, starting from Preparation IX (80 mg, 0.09 mmol) as the appropriate carboxylic acid and the product of Step B (20 mg, 1.0 eq.) as the appropriate amine, afforded the title compound (57 mg, 65%).

HRMS-ESI (m/z) [M+H]⁺ calcd for C₅₈H₆₆FN₈O₆: 989.5089, found 989.5083.

### Example 138: N-(5-cyano-1,2-dimethyl-pyrrol-3-yl)-5-[2-[2-[4-[2-[cyclopropyl(methyl)amino]ethoxy]phenyl]acetyl]-7-[(3S)-3-[[isopropyl(methyl)amino]methyl]-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

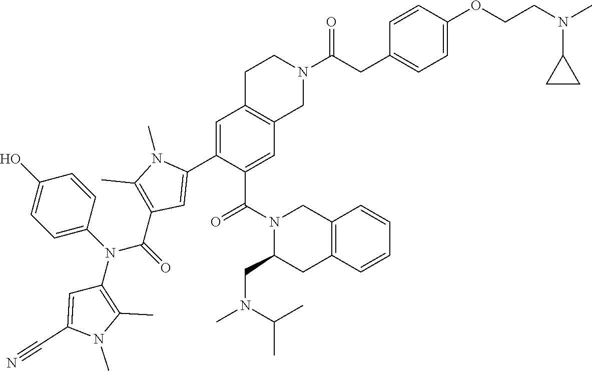 [SMILES: Cc1c(N(C(=O)c2cc(-c3cc4c(cc3C(=O)N3Cc5ccccc5C[C@H]3CN(C)C(C)C)CN(C(=O)Cc3ccc(OCCN(C)C5CC5)cc3)CC4)n(C)c2C)c2ccc(O)cc2)cc(C#N)n1C]

### Step A: tert-butyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-2-[2-[4-[2-[cyclopropyl(methyl)amino]ethoxy]phenyl]acetyl]-3,4-dihydro-1H-isoquinoline-7-carboxylate

According to the amide coupling step of General Procedure 4c, starting from the product of Step G of Example 35 (92 mg, 1.5 eq.) as the appropriate acid and the product of Step D of Preparation IX (415 mg, 0.598 mmol) as the appropriate amine, followed with a purification step via automated flash chromatography using DCM and methanol as eluents afforded the title product (550 mg, 99%).

HRMS-ESI (m/z) [M+H]⁺ calcd for C₅₄H₆₉N₆O₆Si: 925.5042, found 925.5038.

### Step B: 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-2-[2-[4-[2-[cyclopropyl(methyl)amino]ethoxy]phenyl]acetyl]-3,4-dihydro-1H-isoquinoline-7-carboxylic acid

Using General Procedure 3a, starting from the product of Step A (550 mg, 0.59 mmol), followed with a purification step via automated flash chromatography using DCM and methanol as eluents afforded the title product (150 mg, 29%).

¹H NMR (500 MHz, DMSO-d6) δ ppm 12.5 (brs, 1H), 9.83 (brs, 1H), 7.70-6.71 (m, 11H), 5.10 (s, 1H), 5.00-2.10 (m, 16H), 3.57/3.13 (s, 6H), 2.38/1.97 (m, 6H), 1.12-0.68 (m, 4H), 0.90 (s, 9H), 0.14 (s, 6H).

HRMS-ESI (m/z) [M+H]⁺ calcd for C₅₀H₆₁N₆O₆Si: 869.4416, found 869 4416.

### Step C: N-(5-cyano-1,2-dimethyl-pyrrol-3-yl)-5-[2-[2-[4-[2-[cyclopropyl(methyl)amino]ethoxy]phenyl]acetyl]-7-[(3S)-3-[[isopropyl(methyl)amino]methyl]-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

Using General Procedure 4c, starting from the product of Step B (75 mg, 0.086 mmol) as the appropriate acid and the compound obtained in Step B of Example 91 (25 mg, 1.3 eq.) as the appropriate amine, afforded the title product (49 mg, 59%).

HRMS-ESI (m/z) [M+H]⁺ calcd for C₅₈H₆₇N₈O₅: 955.5228, found 955.5219.

### Example 139: N-(5-cyano-1,2-dimethyl-pyrrol-3-yl)-5-[2-[2-[4-[2-[cyclopropyl(methyl)amino]ethoxy]phenyl]acetyl]-7-[(3S)-3-[[(3S)-3-methyl-1-piperidyl]methyl]-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

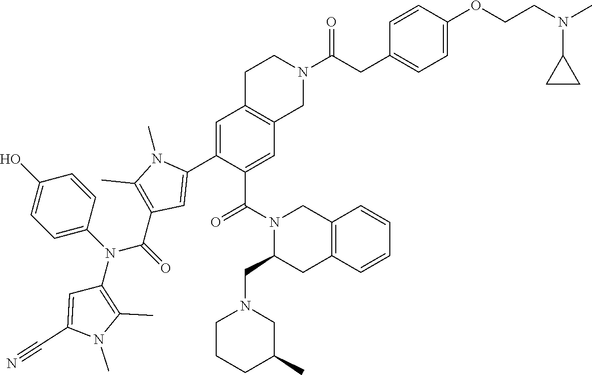 [SMILES: Cc1c(N(C(=O)c2cc(-c3cc4c(cc3C(=O)N3Cc5ccccc5C[C@H]3CN3CCC[C@H](C)C3)CN(C(=O)Cc3ccc(OCCN(C)C5CC5)cc3)CC4)n(C)c2C)c2ccc(O)cc2)cc(C#N)n1C]

Using General Procedure 4c, starting from the compound obtained in Step B of Example 138 (75 mg, 0.086 mmol) as the appropriate acid and the compound obtained in Step A of Example 107 (30 mg. 1.2 eq.) as the appropriate amine, afforded the title product (38 mg, 49%).

HRMS-ESI (m/z) [M+H]⁺ calcd for C₆₀H₆₉N₈O₅: 981.5385, found 981.5386.

### Example 140: N-(5-cyano-1,2-dimethyl-1H-pyrrol-3-yl)-5-[2-(2-{2-fluoro-4-[2-(morpholin-4-yl)ethoxyphenyl}acetyl)-7-[(3S)-3-[(4-methylpiperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

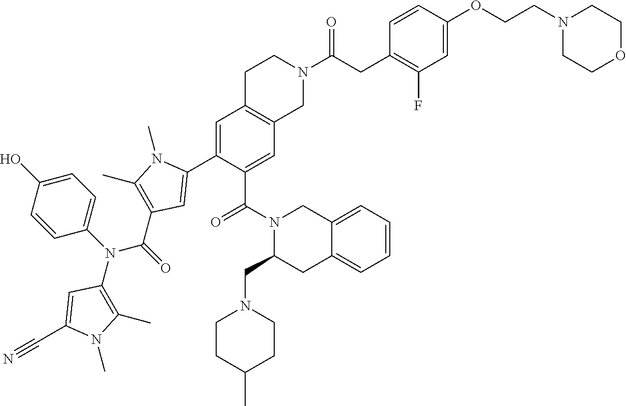 [SMILES: Cc1c(N(C(=O)c2cc(-c3cc4c(cc3C(=O)N3Cc5ccccc5C[C@H]3CN3CCC(C)CC3)CN(C(=O)Cc3ccc(OCCN5CCOCC5)cc3F)CC4)n(C)c2C)c2ccc(O)cc2)cc(C#N)n1C]

### Step A: tert-butyl (3S)-3-(4-methylpiperidine-1-carbonyl)-3,4-dihydro-1H-isoquinoline-2-carboxylate

Using General procedure 5a, starting from (3S)-2-tert-butoxycarbonyl-3,4-dihydro-]H-isoquinoline-3-carboxylic acid (700 mg, 2.53 mmol) and 4-methylpiperidine (300 mg, 1.2 eq), the title compound (642 mg, 71%) was obtained.

¹H NMR (500 MHz, DMSO-d6) δ ppm 7.37-6.97 (m, 4H), 5.28-4.80 (dd, 1H), 4.76-2.38 (brm, 8H), 1.85-0.80 (m, 4H), 1.61 (m, 1H), 1.50-1.27 (brs, 9H), 0.92 (brd, 3H).

HRMS-ESI (m/z) [M+H]⁺ calcd for C₂₁H₃₁N₂O₃: 359.2329, found 359.2323.

### Step B: (4-methyl-1-piperidyl)-[(3S)-1,2,3,4-tetrahydroisoquinolin-3-yl]methanone hydrochloride

Using General procedure 6c, starting from the product of Step A (632 mg, 1.76 mmol) the desired product (483 mg, 93%) was obtained.

¹H NMR (500 MHz, DMSO-d6) δ ppm 9.98/9.37 (m, 2H), 7.32-7.19 (m, 4H), 4.77 (dd, 1H), 4.37/3.88/3.10/2.69 (m+m, 4H), 4.29 (m, 2H), 3.20/3.15/2.97/2.91 (dd+dd, 2H), 1.80-0.92 (m, 4H), 1.66 (m, 1H), 0.95/0.92 (d, 3H).

HRMS-ESI (m/z) [M+H]⁺ calcd for C₁₆H₂₃N₂O: 259.1805, found 259.1799.

### Step C: (3S)-3-[(4-methyl-1-piperidyl)methyl]-1,2,3,4-tetrahydroisoquinoline

Using General procedure 6a, starting from the product of Step B (470 mg, 1.6 mmol), the title product (218 mg, 56%) was obtained.

¹H NMR (500 MHz, DMSO-d6) δ ppm 7.12-6.98 (m, 4H), 3.91 (s, 2H), 3.38/2.66 (dd+dd, 2H), 2.95 (m, 1H), 2.87/2.78/1.99/1.86 (m+m, 4H), 2.34/2.28 (dd+dd, 2H), 1.57/1.16 (m+m, 4H), 1.32 (m, 1H), 0.89 (d, 3H); ¹³C NMR (125 MHz, DMSO-d6) δ ppm 64.3, 55.2/53.9, 50.9, 48.3, 34.6/34.5, 34.1, 30.9, 22.4.

HRMS-ESI (m/z) [M+H]⁺ calcd for C₁₆H₂₅N₂: 245.2012, found 245.2010.

### Step D: N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-[(4-methylpiperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide

Using General procedure 4c, starting from Preparation IX (97 mg, 0.11 mmol) as the appropriate carboxylic acid and the product from Step C (31 mg, 1 eq.) as the appropriate amine, afforded the title product (71.1 mg, 65%).

HRMS-ESI (m/z) [M+H]⁺ calcd for C₆₀H₆₈FN₈O₆: 1015.5240, found 1015.5233.

### Example 141: N-(5-cyano-1,2-dimethyl-1H-pyrrol-3-yl)-5-[2-(2-{2-fluoro-4-[(2R or S)-2-(morpholin-4-yl)propoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 2)

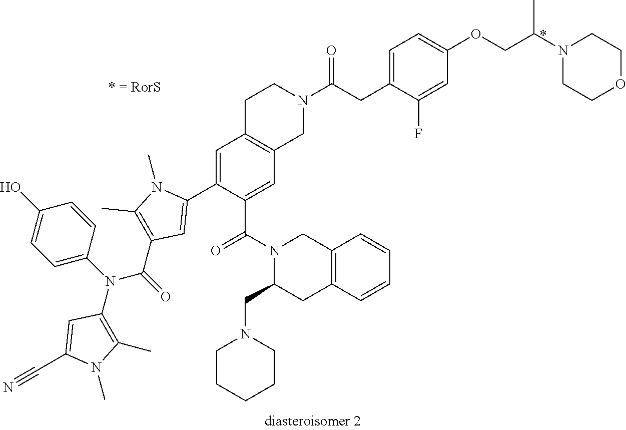 [SMILES: Cc1c(N(C(=O)c2cc(-c3cc4c(cc3C(=O)N3Cc5ccccc5C[C@H]3CN3CCCCC3)CN(C(=O)Cc3ccc(OCC(C)N5CCOCC5)cc3F)CC4)n(C)c2C)c2ccc(O)cc2)cc(C#N)n1C]

### Step A: 2-[2-fluoro-4-((2R or S)-2-morpholinopropoxy)phenyl]acetic acid (from Enantiomer 2)

Using the General procedure 8, starting from the Enantiomer 2 of Step A of Example 135 (380 mg, 1.22 mmol), the desired product was obtained (290 mg, 79%).

¹H NMR (400 MHz, DMSO-d6) δ ppm 12.41 (br., 1H), 7.20 (t, 1H), 6.81 (dd, 1H), 6.74 (dd, 1H), 4.05/3.86 (dd+dd, 2H), 3.55 (t, 4H), 3.51 (s, 2H), 2.86 (m, 1H), 2.55 (m, 4H), 1.06 (d, 3H); ¹³C NMR (100 MHz, DMSO-d6) δ ppm 172.5, 161.5, 159.4, 132.6, 114.6, 111.0, 102.3, 70.3, 67.1, 58.2, 49.7, 34.0, 13.2.

HRMS-ESI (m/z) [M+H]⁺ calcd for C₁₅H₂₁FNO₄: 298.1449, found 298.1450.

### Step B: N-(5-cyano-1,2-dimethyl-1H-pyrrol-3-yl)-5-[2-(2-{2-fluoro-4-[(2R or S)-2-(morpholin-4-yl)propoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide (Diastereomer 2)

Using General procedure 4c, starting from the product of Step A (80 mg, 2.9 eq.) as the appropriate carboxylic acid and Preparation VIIa (80 mg, 0.09 mmol) as the appropriate amine, afforded the title compound (53 mg, 55%).

HRMS-ESI (m/z) [M+H]⁺ calcd for C₆₀H₆₈FN₈O₆: 1015.5246, found 1015.5245.

### Example 142: N-(4-hydroxyphenyl)-1,2-dimethyl-N-[5-methyl-1-(oxan-4-yl)pyrazol-4-yl]-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxamide

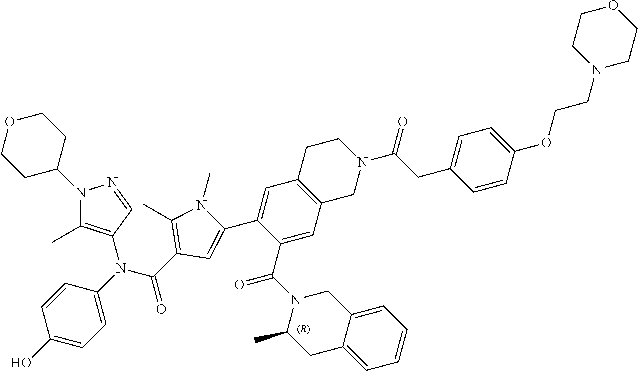

### Step A: N-{4-[(tert-butyldimethylsilyl)oxy]phenyl}-5-methyl-1-(oxan-4-yl)pyrazol-4-amine

Using General procedure 1a and a mixture of 4-bromo-3-methyl-1-(oxan-4-yl)pyrazole and 4-bromo-5-methyl-1-(oxan-4-yl)pyrazole (300 mg, 0.61 mmol, 1 eq.) as the appropriate aryl bromide and Preparation IIa (273 mg, 1.22 mmol, 2 eq.) as the appropriate aniline, a mixture of products was obtained. Purification by automated flash chromatography eluting with a gradient of 0-60% EtOAc in heptane afforded the title product (158 mg, 0.41 mmol, 67%).

LRMS calcd for C₂₁H₃₃N₃O₂Si: 387.2, found 388.3 [M+H]⁺

¹H NMR (400 MHz, DMSO-d6) δ ppm: 7.28 (s, 1H), 6.70 (s, 1H), 6.61-6.56 (m, 2H), 6.49-6.44 (m, 2H), 4.38-4.27 (m, 1H), 4.00-3.91 (m, 2H), 3.53-3.43 (m, 2H), 2.12 (s, 3H), 2.08-1.95 (m, 2H), 1.82-1.74 (m, 2H), 0.92 (s, 9H), −0.11 (s, 6H).

### Step B: tert-butyl 6-[4-({4-[(tert-butyldimethylsilyl)oxy]phenyl}[5-methyl-1-(oxan-4-yl)pyrazol-4-yl]carbamoyl)-1,5-dimethylpyrrol-2-yl]-7-{[(3R)-3-methyl-3,4-dihydro-1H-isoquinolin-2-yl]carbonyl}-3,4-dihydro-1H-isoquinoline-2-carboxylate

Using General procedure 2a and Preparation Ha (350 mg, 0.64 mmol, 1 eq.) as the appropriate acid and the product from Step A (374 mg, 0.97 mmol, 1.5 eq.) as the appropriate aniline afforded the title product (447 mg, 0.49 mmol, 76%).

LRMS calcd for C₅₃H₆₈N₆O₆Si: 912.5, found 913.6 [M+H]⁺

### Step C: N-(4-hydroxyphenyl)-1,2-dimethyl-N-[5-methyl-1-(oxan-4-yl)pyrazol-4-yl]-5-(7-{[(3R)-3-methyl-3,4-dihydro-1H-isoquinolin-2-yl]carbonyl}-1,2,3,4-tetrahydroisoquinolin-6-yl)pyrrole-3-carboxamide

Using General procedure 3b and the product from Step B (447 mg, 0.49 mmol, 1 eq.) as the appropriate silyl-derivative afforded the title product (236 mg, 0.34 mmol, 69%).

LRMS calcd for C₄₂H₄₆N₆O₄: 698.4, found 699.6 [M+H]⁺

### Step D: N-(4-hydroxyphenyl)-1,2-dimethyl-N-[5-methyl-1-(oxan-4-yl)pyrazol-4-yl]-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxamide

Using General procedure 4a and the product from Step C (236 mg, 0.34 mmol, 1 eq.) as the appropriate amine and Preparation Va (108 mg, 0.41 mmol, 1.2 eq.) as the appropriate carboxylic acid afforded the title product (9.9 mg, 0.01 mmol, 3%).

HRMS calcd for C₅₆H₆₃N₇O₇: 945.4789, found 946.4865 [M+H]⁺

### Example 143: N-(4-hydroxyphenyl)-N-[1-(2-methoxyethyl)pyrazol-4-yl]-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxamide

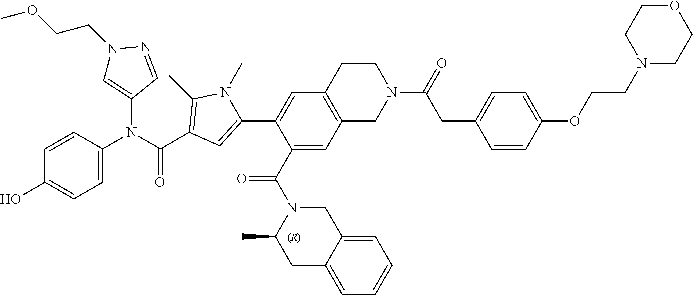

### Step A: N-{4-[(tert-butyldimethylsilyl)oxy]phenyl}-1-(2-methoxyethyl)pyrazol-4-amine

Using General procedure 1a and 4-bromo-1-(2-methoxyethyl)-1H-pyrazole (350 mg, 1.71 mmol, 1 eq.) as the appropriate aryl bromide and Preparation IIIa (419 mg, 1.88 mmol, 1.1 eq.) as the appropriate aniline afforded the title product (305 mg, 0.88 mmol, 51%).

LRMS calcd for C18H₂₉N₃O₂Si: 347.2, found 348.4 [M+H]⁺

¹H NMR (400 MHz, DMSO-d6) δ ppm: 7.56 (d, J=0.9 Hz, 1H), 7.26 (d, J=0.9 Hz, 1H), 7.21 (s, 1H), 6.69-6.61 (m, 4H), 4.18 (t, J=5.4 Hz, 2H), 3.66 (t, J=5.4 Hz, 2H), 3.23 (s, 3H), 0.93 (s, 9H), 0.13 (s, 6H).

### Step B: tert-butyl 6-[4-({4-[(tert-butyldimethylsilyl)oxy]phenyl}[1-(2-methoxyethyl)pyrazol-4-yl]carbamoyl)-1,5-dimethylpyrrol-2-yl]-7-{[(3R)-3-methyl-3,4-dihydro-1H-isoquinolin-2-yl]carbonyl}-3,4-dihydro-1H-isoquinoline-2-carboxylate

Using General procedure 2a and Preparation Ha (400 mg, 0.74 mmol, 1 eq.) as the appropriate acid and the product from Step A (384 mg, 1.1 mmol, 1.5 eq.) as the appropriate aniline afforded the title product (591 mg, 0.68 mmol, 92%).

LRMS calcd for C₅₀H₆₄N₆O₆Si: 872.5, found 873.6 [M+H]⁺

### Step C: N-(4-hydroxyphenyl)-N-[1-(2-methoxyethyl)pyrazol-4-yl]-1,2-dimethyl-5-(7-{[(3R)-3-methyl-3,4-dihydro-1H-isoquinolin-2-yl]carbonyl}-1,2,3,4-tetrahydroisoquinolin-6-yl)pyrrole-3-carboxamide

Using General procedure 3b and the product from Step B (591 mg, 0.68 mmol, 1 eq.) as the appropriate silyl-derivative afforded the title product (347 mg, 0.53 mmol, 78%).

LRMS calcd for C₃₉H₄₂N₆O₄: 658.3, found 659.6 [M+H]⁺

### Step D: N-(4-hydroxyphenyl)-N-[1-(2-methoxyethyl)pyrazol-4-yl]-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxamide

Using General procedure 4a and the product from Step C (347 mg, 0.53 mmol, 1 eq.) as the appropriate amine and Preparation Va (168 mg, 0.63 mmol, 1.2 eq.) as the appropriate carboxylic acid afforded the title product (66 mg, 0.07 mmol, 14%).

HRMS calcd for C₅₃H₅₉N₇O₇: 905.4476, found 906.4550 [M+H]⁺

### Example 144: N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-[1-(2-methylpropyl)pyrazol-4-yl]pyrrole-3-carboxamide

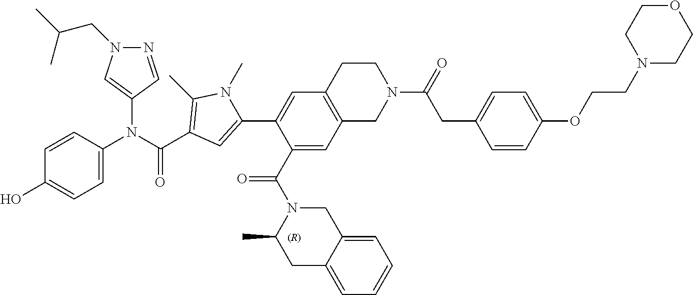

### Step A: N-{4-[(tert-butyldimethylsilyl)oxy]phenyl}-1-(2-methylpropyl)pyrazol-4-amine

Using General procedure 1a and 4-bromo-1-isobutylpyrazole (300 mg, 1.48 mmol, 1 eq.) as the appropriate aryl bromide and Preparation IIa (363 mg, 1.62 mmol, 1.1 eq.) as the appropriate aniline afforded the title product (256 mg, 0.74 mmol, 50%).

LRMS calcd for C₁₉H₃₁N₃OSi: 345.2, found 346.4 [M+H]⁺

¹H NMR (400 MHz, DMSO-d6) δ ppm: 7.57 (d, J=0.9 Hz, 1H), 7.25 (d, J=0.9 Hz, 1H), 7.19 (s, 1H), 6.68-6.61 (m, 4H), 3.83 (d, J=7.3 Hz, 2H), 2.16-2.01 (m, 1H), 0.93 (s, 9H), 0.83 (d, J=6.7 Hz, 6H), 0.12 (s, 6H).

### Step B: tert-butyl 6-[4-({4-[(tert-butyldimethylsilyl)oxy]phenyl}[1-(2-methylpropyl)pyrazol-4-yl]carbamoyl)-1,5-dimethylpyrrol-2-yl]-7-{[(3R)-3-methyl-3,4-dihydro-1H-isoquinolin-2-yl]carbonyl}-3,4-dihydro-1H-isoquinoline-2-carboxylate

Using General procedure 2a and Preparation IIa (350 mg, 0.64 mmol, 1 eq.) as the appropriate acid and the product from Step A (267 mg, 0.77 mmol, 1.2 eq.) as the appropriate aniline afforded the title product (386 mg, 0.44 mmol, 69%).

LRMS calcd for C₅₁H₆₆N₆O₅Si: 870.5, found 871.6 [M+H]⁺

### Step C: N-(4-hydroxyphenyl)-1,2-dimethyl-5-(7-{[(3R)-3-methyl-3,4-dihydro-1H-isoquinolin-2-yl]carbonyl}-1,2,3,4-tetrahydroisoquinolin-6-yl)-N-[1-(2-methylpropyl)pyrazol-4-yl]pyrrole-3-carboxamide

Using General procedure 3b and the product from Step B (386 mg, 0.22 mmol, 1 eq) as the appropriate silyl-derivative afforded the title product (106 mg, 0.16 mmol, 73%).

LRMS calcd for C₄₀H₄₄N₆O₃: 656.3, found 657.4 [M+H]⁺

### Step D: N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-[1-(2-methylpropyl)pyrazol-4-yl]pyrrole-3-carboxamide

Using General procedure 4a and the product from Step C (106 mg, 0.16 mmol, 1 eq.) as the appropriate amine and Preparation Va (51 mg, 0.19 mmol, 1.2 eq.) as the appropriate carboxylic acid afforded the title product (14 mg, 0.02 mmol, 10%).

HRMS calcd for C₅₄H₆₁N₇O₆: 903.4683, found 904.4758 [M+H]⁺

### Example 145: 5-{7-[(3S)-3-({5-azaspiro[2.3]hexan-5-yl}methyl)-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

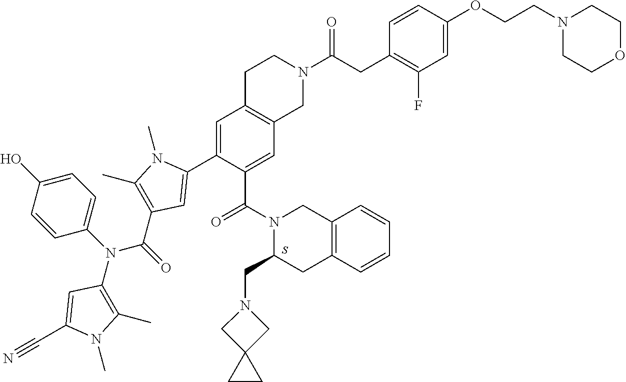

### Step A: tert-butyl (3S)-3-(5-azaspiro[2.3]hexane-5-carbonyl)-3,4-dihydro-1H-isoquinoline-2-carboxylate

Using General procedure 5a, starting from (3S)-2-tert-butoxycarbonyl-3,4-dihydro-1H-isoquinoline-3-carboxylic acid (700 mg, 2.53 mmol) and 5-azaspiro[2.3]hexane hydrochloride (400 mg, 1.3 eq) the title product (730 mg, 84%) was obtained.

¹H NMR (500 MHz, DMSO-d6) δ ppm 7.28-7.12 (m, 4H), 4.79/4.39 (dd, 1H), 4.65/4.65/4.40/4.26 (d+d, 2H), 4.32-3.76 (m, 4H), 3.16-2.82 (dd+dd, 2H), 1.44/1.41 (brs, 9H), 0.71-0.56 (m, 4H).

HRMS-ESI (m/z) [M+H]⁺ calcd for C₂₀H₂₇N₂O₃: 343.2016, found 343.2015.

### Step B: 5-azaspiro[2.3]hexan-5-yl-[(3S)-1,2,3,4-tetrahydroisoquinolin-3-yl]methanone hydrochloride

Using General procedure 6c, starting from the product of Step A (721 mg, 2.1 mmol), the title product (528 mg, 90%) was obtained.

¹H NMR (500 MHz, DMSO-d6) δ ppm 10.13/9.52 (br+br., 2H), 7.36-7.17 (m, 4H), 4.40/4.36/4.08/4.01 (d+d/d+d, 4H), 4.37 (br., 1H), 4.32-4.26 (br., 2H), 3.26/2.98 (dd+dd, 2H), 0.69 (br., 4H); ¹³C NMR (125 MHz, DMSO-d6) δ ppm 167.4, 58.2/56.2, 51.6, 44.3, 28.5, 15-6, 9.6.

HRMS-ESI (m/z) [M+H]⁺ calcd for C₁₅H₁₉N₂O: 243.1492, found 243.1492.

### Step C: (3S)-3-(5-azaspiro[2.3]hexan-5-ylmethyl)-1,2,3,4-tetrahydroisoquinoline

Using General procedure 6a, starting from the product of Step B (490 mg, 1.75 mmol), the title compound (223 mg, 55%) was obtained.

¹H NMR (400 MHz, DMSO-d6) δ ppm 7.13-6.96 (m, 4H), 3.89 (s, 2H), 3.31/3.27 (d+d, 4H), 2.71 (m, 1H), 2.67/2.41 (dd+dd, 2H), 2.56/2.53 (dd+dd, 2H), 0.48 (s, 4H); ¹³C NMR (100 MHz, DMSO-d6) δ ppm 65.2, 63, 52.4, 48.2, 33.7, 16.6, 9.3.

HRMS-ESI (m/z) [M+H]⁺ calcd for C₁₅H₂₁N₂: 229.1699, found 229.1697.

### Step D: 5-{7-[(3S)-3-({5-azaspiro[2.3]hexan-5-yl}methyl)-1,2,3,4-tetrahydroisoquinoline-2-carbonyl]-2-(2-{2-fluoro-4-[2-(morpholin-4-yl)ethoxy]phenyl}acetyl)-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide

Using General procedure 4c, starting from Preparation IX (95 mg, 0.11 mmol) as the appropriate carboxylic acid and the product from Step C (29 mg, 1 eq.) as the appropriate amine, afforded the title compound (67.2 mg, 64%).

HRMS-ESI (m/z) [M+H]⁺ calcd for C₅₉H₆₄FN₈O₆: 999.4927, found 999.4930.

### Example 146: N-(5-cyano-1,2-dimethylpyrrol-3-yl)-5-(7-{[(3S)-3-[(3,3-dimethylpyrrolidin-1-yl)methyl]-3,4-dihydro-1H-isoquinolin-2-yl]carbonyl}-2-(2-{2-fluoro-4-[2-(morpholin-4-yl)ethoxy]phenyl}acetyl)-3,4-dihydro-1H-isoquinolin-6-yl)-N-(4-hydroxyphenyl)-1,2-dimethylpyrrole-3-carboxamide

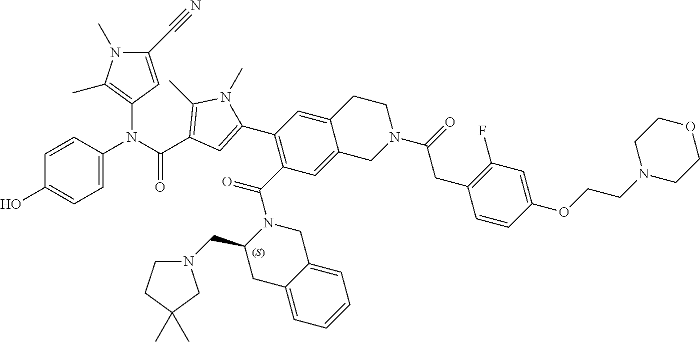

### Step A: tert-butyl (3S)-3-[(3,3-dimethylpyrrolidin-1-yl)carbonyl]-3,4-dihydro-1H-isoquinoline-2-carboxylate

Using General procedure 5 and Boc-(3S)-1,2,3,4-tetrahydroisoquinoline-3-carboxylic acid (500 mg, 1.8 mmol, 1 eq.) and 3,3-dimethylpyrrolidine (0.23 mL, 1.89 mmol, 1.05 eq.) as the appropriate amine afforded the title product (646 mg, quant.).

LRMS calcd for C₂₁H₃₀N₂O₃: 358.2, found 359.4 [M+H]⁺

¹H NMR (400 MHz, DMSO-d6) δ ppm: 7.27-7.08 (m, 4H), 4.92-4.20 (m, 3H), 3.70-2.76 (m, 6H), 1.83-1.49 (m, 2H), 1.49-1.28 (m, 9H), 1.13-0.99 (m, 6H).

### Step B: (3S)-3-[(3,3-dimethylpyrrolidin-1-yl)methyl]-1,2,3,4-tetrahydroisoquinoline dihydrochloride

Using General procedure 6 and the product from Step A (646 mg, 1.8 mmol, 1 eq.) as the appropriate THIQ amide afforded the title product (572 mg, quant.).

LRMS calcd for C₁₆H₂₄N₂: 244.2, found 245.2 [M+H]⁺

¹H NMR (400 MHz, DMSO-d6) δ ppm: 11.38-10.67 (br s 1H), 10.50-9.81 (br s, 2H), 7.38-7.14 (m, 4H), 4.51-4.33 (m, 2H), 4.12-2.84 (m, 9H), 2.01-1.66 (m, 2H), 1.21-1.12 (m, 6H).

### Step C. N-(5-cyano-1,2-dimethylpyrrol-3-yl)-5-(7-{[(3S)-3-[(3,3-dimethylpyrrolidin-1-yl)methyl]-3,4-dihydro-1H-isoquinolin-2-yl]carbonyl}-2-(2-{2-fluoro-4-[2-(morpholin-4-yl)ethoxy]phenyl}acetyl)-3,4-dihydro-1H-isoquinolin-6-yl)-N-(4-hydroxyphenyl)-1,2-dimethylpyrrole-3-carboxamide

Using General procedure 4a and Preparation IX (100 mg, 0.11 mmol, 1 eq.) as the appropriate carboxylic acid and the product from Step B (39 mg, 0.12 mmol, 1.1 eq.) as the appropriate amine afforded an intermediate which was treated according to General procedure 3c to afford the title product (16 mg, 0.02 mmol, 14%).

HRMS calcd for C₆₀H₆₇FN₈O₆: 1014.5168, found 1015.5237 [M+H]⁺

### Example 147: N-(4-hydroxyphenyl)-1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-{4H,5H,6H,7H-pyrazolo[1,5-a]pyridin-3-yl}pyrrole-3-carboxamide

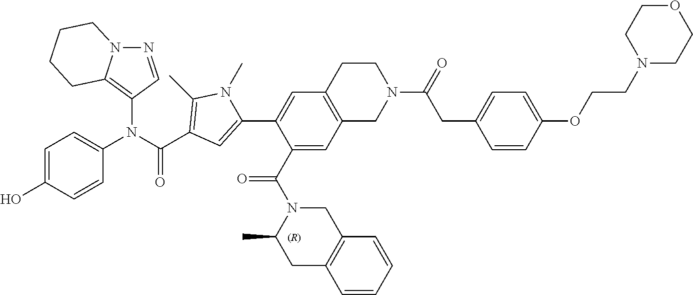

### Step A: N-{4-[(tert-butyldimethylsilyl)oxy]phenyl}-4H,5H,6H, 7H-pyrazolo[1,5-a]pyridin-3-amine

Using General procedure 1a and 3-bromo-4H,5H,6H,7H-pyrazolo[1,5-a]pyridine (314 mg, 1.56 mmol, 1 eq.) as the aryl bromide and Preparation IIIa (384 mg, 1.72 mmol, 1.1 eq.) as the appropriate aniline afforded the title product (323 mg, 0.94 mmol, 60%).

LRMS calcd for C₁₉H₂₉N₃OSi: 343.2, found 344.4 [M+H]⁺

¹H NMR (400 MHz, DMSO-d6) δ ppm: 7.25 (s, 1H), 6.69 (s, 1H), 6.62-6.56 (m, 2H), 6.51-6.46 (m, 2H), 4.04-3.98 (m, 2H), 1.98-1.89 (m, 2H), 1.80-1.70 (m, 2H), 0.92 (s, 9H), 0.11 (s, 6H).

### Step B: tert-butyl 6-[4-({4-[(tert-butyldimethylsilyl)oxy]phenyl}({4H,5H,6H,7H-pyrazolo[1,5-a]pyridin-3-yl})carbamoyl)-1,5-dimethylpyrrol-2-yl]-7-{[(3R)-3-methyl-3,4-dihydro-1H-isoquinolin-2-yl]carbonyl}-3,4-dihydro-1H-isoquinoline-2-carboxylate

Using General procedure 2a and Preparation Ha (350 mg, 0.64 mmol, 1 eq.) as the appropriate acid and the product from Step A (310 mg, 0.9 mmol, 1.4 eq.) as the appropriate aniline afforded the title product (480 mg, 0.55 mmol, 86%).

LRMS calcd for C₅₁H₆₄N₆O₅Si: 868.5, found 869.6 [M+H]⁺

### Step C: N-(4-hydroxyphenyl)-1,2-dimethyl-5-(7-f[(3R)-3-methyl-3,4-dihydro-1H-isoquinolin-2-yl]carbonyl}-1,2,3,4-tetrahydroisoquinolin-6-yl)-N-{4H,5H,6H, 7H-pyrazolo[1,5-a]pyridin-3-yl}pyrrole-3-carboxamide

Using General procedure 3b and the product from Step B (480 mg, 0.55 mmol, 1 eq.) as the appropriate silyl-derivative afforded the title product (306 mg, 0.47 mmol, 85%).

LRMS calcd for C₄₀H₄₂N₆O₃: 654.3, found 655.4 [M+H]⁺

### Step D: N-(4-hydroxyphenyl)-1,2-dimethyl-5-(7-f[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)-N-{4H,5H,6H, 7H-pyrazolo[1,5-a]pyridin-3-yl}pyrrole-3-carboxamide

Using General procedure 4a and the product from Step C (306 mg, 0.47 mmol, 1 eq.) as the appropriate amine and Preparation Va (124 mg, 0.47 mmol, 1 eq.) as the appropriate carboxylic acid afforded the title product (28.3 mg, 0.03 mmol, 7%).

HRMS calcd for C₅₄H₅₉N₇O₆: 901.4527, found 902.4598 [M+H]⁺

### Example 148: N-(5-cyano-1,2-dimethylpyrrol-3-yl)-5-[2-(2-{4-[2-(3,3-dimethylmorpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

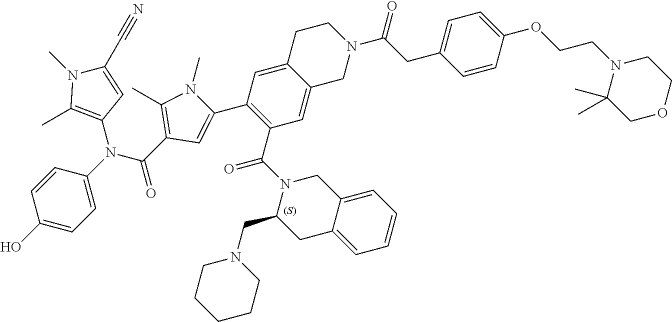

### Step A: methyl 2-{4-[2-(3,3-dimethylmorpholin-4-yl)ethoxy]phenyl}acetate

A mixture of the product from Example 112, Step A (830 mg, 3.63 mmol, 1 eq.), K₂CO₃ (2.01 g, 14.52 mmol, 4 eq.), NaI (272 mg, 1.81 mmol, 0.5 eq.) and 3,3-dimethylmorpholine (460 mg, 3.99 mmol, 1.1 eq.) in MeCN (35 mL) was refluxed for 18 h. The mixture was allowed to cool to rt, filtered and washed with EtOAc. The filtrate was concentrated in vacuo and purified by automated flash chromatography eluting with a gradient of 0-100% EtOAc in heptane to afford the title product (69.5 mg, 0.23 mmol, 6%).

LRMS calcd for C₁₇H₂₅NO₄: 307.2, found 308.4 [M+H]⁺

¹H NMR (400 MHz, DMSO-d6) δ ppm: 7.18-7.11 (m, 2H), 6.89-6.82 (m, 2H), 3.94 (t, J=6.3 Hz, 2H), 3.62-3.55 (m, 7H), 3.20 (s, 2H), 2.69-2.62 (m, 2H), 2.62-2.56 (m, 2H), 0.95 (s, 6H).

### Step B: 2-{4-[2-(3,3-dimethylmorpholin-4-yl)ethoxy]phenyl}acetic acid

Using General procedure 8 and the product from Step A (69.5 mg, 0.23 mmol, 1 eq.) as the appropriate ester afforded the title product (49.2 mg, 0.17 mmol, 74%).

LRMS calcd for C₁₆H₂₃NO₄: 293.2, found 294.2 [M+H]⁺

¹H NMR (400 MHz, DMSO-d6) δ ppm: 9.64-9.47 (m, 1H), 7.21-7.15 (m, 2H), 6.94-6.87 (m, 2H), 4.36-4.20 (m, 2H), 3.99-3.89 (m, 1H), 3.80-3.68 (m, 2H), 3.67-3.50 (m, 3H), 3.49-3.44 (m, 2H), 3.43-3.19 (m, 2H), 1.35 (s, 3H), 1.28 (s, 3H).

### Step C. N-(5-cyano-1,2-dimethylpyrrol-3-yl)-5-[2-(2-{4-[2-(3,3-dimethylmorpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and Preparation IVb (125 mg, 0.17 mmol, 1 eq.) as the appropriate amine and the product from Step B (50 mg, 0.17 mmol, 1.1 eq.) as the appropriate carboxylic acid afforded the title product (76.3 mg, 0.08 mmol, 44%).

HRMS calcd for C₆₁H₇₀N₈O₆: 1010.5418, found 1011.5492 [M+H]⁺

### Example 149: N-(5-cyano-1,2-dimethyl-1H-pyrrol-3-yl)-N-(4-hydroxyphenyl)-1,2-dimethyl-5-{2-[2-(4-{[(oxan-4-yl)methoxy]methyl}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-1H-pyrrole-3-carboxamide

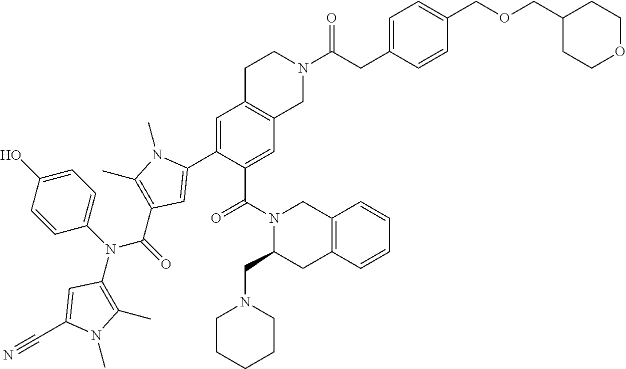 [SMILES: Cc1c(N(C(=O)c2cc(-c3cc4c(cc3C(=O)N3Cc5ccccc5C[C@H]3CN3CCCCC3)CN(C(=O)Cc3ccc(COCC5CCOCC5)cc3)CC4)n(C)c2C)c2ccc(O)cc2)cc(C#N)n1C]

### Step A: tert-butyl-[2-[4-(chloromethyl)phenyl]ethoxy]-dimethyl-silane

To a solution of 2-[4-(chloromethyl)phenyl]ethanol (1.00 g, 5.86 mmol) in DCM (5 mL/mmol) was added imidazole (1.60 g, 4 eq.) at 0° C., followed by the addition of tert-butyl-chloro-dimethyl-silane (1.77 g, 2 eq.). The reaction mixture was allowed to warm to room temperature was stirred for 2 h. After completion, the reaction mixture was diluted with DCM (75 mL), washed with sat. NaHCO₃ solution (2×50 mL), water (75 mL) and brine (75 mL). The organic layer was dried over sodium sulphate, filtered and evaporated to obtain the title product (1.65 g 99%).

¹H NMR (400 MHz, DMSO-d6) δ ppm 7.33 (dm, 2H), 7.22 (dm, 2H), 4.72 (s, 2H), 3.76 (t, 2H), 2.74 (t, 2H), 0.82 (s, 9H), −0.05 (s, 6H); ¹³C NMR (100 MHz, DMSO-d6) δ ppm 139.8, 135.8, 129.8, 129.1, 64.1, 46.6, 39, 26.3, −5.0.

HRMS-ESI (m/z) [M-C4H9]⁺ calcd for C₁₁H₁₆ClOSi: 227.0653, found 227.0650.

### Step B: 2-[4-(tetrahydropyran-4-ylmethoxymethyl)phenyl]ethanol

To a suspension of sodium hydride (60% dispersion in mineral oil) (90 mg, 1.5 eq.) in DMF (5.7 mL) was added a solution of tetrahydropyran-4-ylmethanol (260 mg, 1.5 eq.) in DMF (1.7 mL) and the mixture was stirred for 30 min. Then the mixture was cooled to 0° C. and a solution of the compound obtained in Step A (430 mg, 1.50 mmol) in DMF (2.5 mL) was added slowly, then the mixture was stirred for 16 h (by the end of this period the silyl group cleaved). The reaction mixture was quenched with sat. ammonium chloride solution, extracted with DCM (3×15 mL), the combined organic layers were washed with brine (20 mL), dried over sodium sulphate, filtered and evaporated. The crude product was purified by flash chromatography using DCM and methanol as eluents to afford the title product (184 mg, 49%).

¹H NMR (400 MHz, DMSO-d6) δ ppm 7.21 (m, 2H), 7.18 (m, 2H), 4.63 (brs, 1H), 4.41 (s, 2H), 3.83/3.27 (m+m, 4H), 3.58 (t, 2H), 3.26 (d, 2H), 2.7 (t, 2H), 1.80 (m, 1H), 1.57/1.19 (m+m, 4H); ¹³C NMR (100 MHz, DMSO-d6) δ ppm 129.2, 127.8, 75, 72.4, 67.2, 62.7, 39.2, 35.4, 30.1.

HRMS-ESI (m/z) [M+H]⁺ calcd for C₁₅H₂₃O₃: 251.1642, found 251.1642.

### Step C: 2-[4-(tetrahydropyran-4-ylmethoxymethyl)phenyl]acetic acid

The product obtained in Step B (184 mg, 0.74 mmol) was dissolved in acetone (6.60 mL), then Jones reagent (0.66 mL, 2.4 eq.) was added dropwise to the solution and the mixture was stirred for 1 h. After completion, the excess Jones reagent was quenched with a few drops of IPA then water (15 mL, 20 mL/mmol substrate) was added. The volatiles were removed under reduced pressure and the aqueous residue was extracted with DCM (5×15 mL), the combined organic layers were dried over magnesium sulphate, filtered and evaporated. The residue was suspended in water (20 mL), the suspension was made basic with 2M aq. sodium hydroxide, and extracted with DCM (2×15 mL) to remove the impurities. Then the aqueous layer was acidified with 2M hydrochloric acid and extracted with DCM (3×15 mL). The combined organic layers were washed with brine (1×15 mL), dried over sodium sulphate, filtered and evaporated to afford the title compound (148 mg, 71%).

¹H NMR (500 MHz, DMSO-d6) δ ppm 12.32 (brs, 1H), 7.25 (m, 2H), 7.23 (m, 2H), 4.43 (s, 2H), 3.82/3.27 (m+m, 4H), 3.55 (s, 2H), 3.27 (d, 2H), 1.81 (m, 1H), 1.58/1.20 (m+m, 4H); ¹³C NMR (125 MHz, DMSO-d6) δ ppm 129.7, 127.8, 75.1, 72.2, 67.2, 40.9, 35.4, 30.0.

HRMS-ESI (m/z) [M+H]⁺ calcd for C₁₅H₂₁O₄: 265.1435, found 265.1434.

### Step D: N-(5-cyano-1,2-dimethyl-1H-pyrrol-3-yl)-N-(4-hydroxyphenyl)-1,2-dimethyl-5-{2-[2-(4-{[(oxan-4-yl)methoxy]methyl}phenyl)acetyl]-7-[(3S)-3-[(piperidin-1-yl)methyl]-1,2,3,4-tetrahydroisoquinoline-2-carbonyl]-1,2,3,4-tetrahydroisoquinolin-6-yl}-1H-pyrrole-3-carboxamide

Using General procedure 4c, starting from the product of Step C (50 mg, 2 eq.) as the appropriate carboxylic acid and Preparation VIIa (80 mg, 0.09 mmol) as the appropriate amine, afforded the title product (48 mg, 52%).

HRMS-ESI (m/z) [M+H]⁺ calcd for C₆₀H₆₈N₇O₆: 982.5226, found 982.5247.

### Example 150: N-(5-cyano-1,2-dimethyl-1H-pyrrol-3-yl)-N-(4-hydroxyphenyl)-1,2-dimethyl-5-[2-(2-{4-[2-((2S)-2-methylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

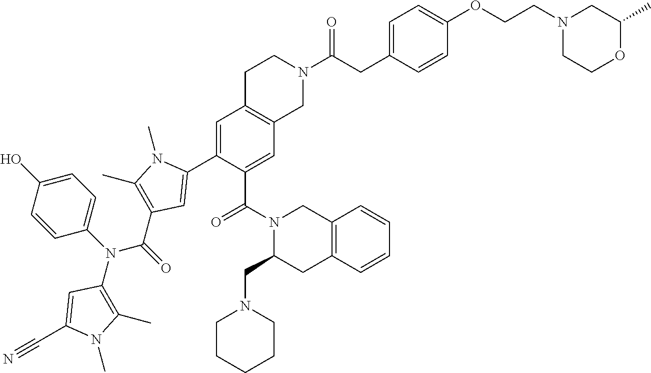 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCO[C@@H](C)C5)cc3)CC4)n(C)c2C)c2ccc(O)cc2)cc(C#N)n1C]

Using General procedure 4c, starting from the compound described at Step A of Example 57 (122 mg, 4 eq.) as the appropriate carboxylic acid and Preparation VIIa (80 mg, 0.09 mmol) as the appropriate amine, afforded the desired compound (51 mg, 55%).

HRMS-ESI (m/z) [M+H]⁺ calcd for C₆₀H₆₉N₈O₆: 997.5334, found 997.5328.

### Example 151: N-(5-cyano-1,2-dimethyl-1H-pyrrol-3-yl)-N-(4-hydroxyphenyl)-1,2-dimethyl-5-[2-(2-{4-[2-((2R)-2-methylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-1H-pyrrole-3-carboxamide

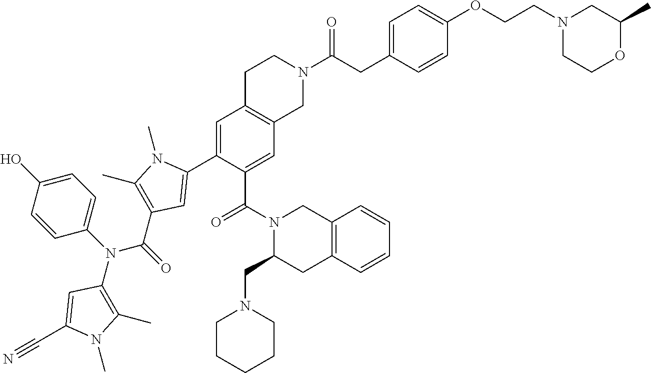 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCO[C@H](C)C5)cc3)CC4)n(C)c2C)c2ccc(O)cc2)cc(C#N)n1C]

Using General procedure 4c, starting from the compound described at Step B of Example 56 (59 mg, 2.0 eq.) as the appropriate carboxylic acid and Preparation VIIa (80 mg, 0.09 mmol) as the appropriate amine, afforded the desired product (36 mg, 39%).

HRMS-ESI (m/z) [M+H]⁺ calcd for C₆₀H₆₉N₈O₆: 997.5334, found 997.5330.

### Example 152: N-(5-cyano-1,2-dimethylpyrrol-3-yl)-5-(7-{[(3S)-3-[(diisopropylamino)methyl]-3,4-dihydro-1H-isoquinolin-2-yl]carbonyl}-2-(2-{2-fluoro-4-[2-(morpholin-4-yl)ethoxy]phenyl}acetyl)-3,4-dihydro-1H-isoquinolin-6-yl)-N-(4-hydroxyphenyl)-1,2-dimethylpyrrole-3-carboxamide

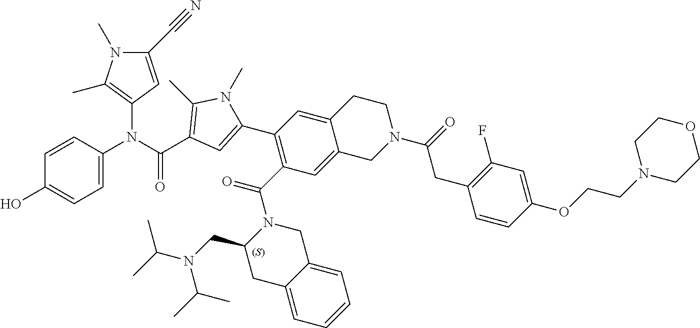

### Step A: tert-butyl (3S)-3-(diisopropylcarbamoyl)-3,4-dihydro-1H-isoquinoline-2-carboxylate

Using General procedure 5 and Boc-(3S)-1,2,3,4-tetrahydroisoquinoline-3-carboxylic acid (500 mg, 1.8 mmol, 1 eq.) and diisopropylamine (0.27 mL, 1.89 mmol, 1.05 eq.) as the appropriate amine afforded the title product (492.7 mg, 1.37 mmol, 76%).

LRMS calcd for C₂₁H₃₂N₂O₃: 360.2, found 361.4 [M+H]⁺

¹H NMR (400 MHz, DMSO-d6) δ ppm: 7.25-7.06 (m, 4H), 4.91-4.05 (m, 3H), 3.52-2.65 (m, 4H), 1.54-1.07 (m, 21H).

### Step B: diisopropyl[(3S)-1,2,3,4-tetrahydroisoquinolin-3-ylmethyl]amine dihydrochloride

Using General procedure 6 and the product from Step A (493 mg, 1.37 mmol, 1 eq.) as the appropriate THIQ amide afforded the title product (436.4 mg, 100%).

LRMS calcd for C₁₆H₂₆N₂: 246.2, found 247.4 [M+H]⁺

### Step C: N-(5-cyano-1,2-dimethylpyrrol-3-yl)-5-(7-{[(3S)-3-[(diisopropylamino)methyl]-3,4-dihydro-1H-isoquinolin-2-yl]carbonyl}-2-(2-{2-fluoro-4-[2-(morpholin-4-yl)ethoxy]phenyl}acetyl)-3,4-dihydro-1H-isoquinolin-6-yl)-N-(4-hydroxyphenyl)-1,2-dimethylpyrrole-3-carboxamide

Using General procedure 4a and Preparation IX (100 mg, 0.11 mmol, 1 eq.) as the appropriate carboxylic acid and the product from Step B (39 mg, 0.12 mmol, 1.1 eq.) as the appropriate amine afforded an intermediate which was treated according to General procedure 3c to afford the title product (4.78 mg, 4.69 μmol, 4%).

HRMS calcd for C₆₀H₆₉FN₈O₆: 1016.5324, found 1017.5397 [M+H]⁺

### Example 153: N-(5-cyano-1,2-dimethyl-pyrrol-3-yl)-N-(4-hydroxyphenyl)-1,2-dimethyl-5-[7-[(3S)-3-[[(3S)-3-methyl-1-piperidyl]methyl]-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

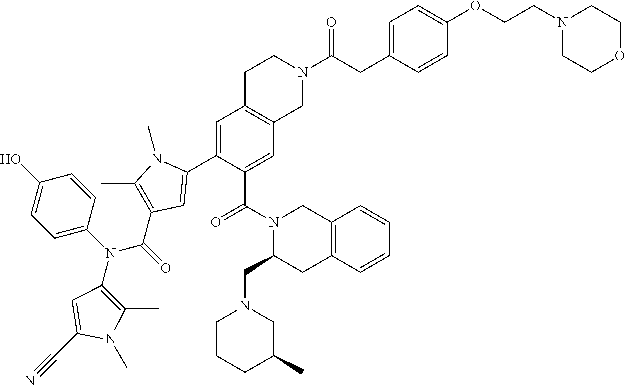 [SMILES: Cc1c(N(C(=O)c2cc(-c3cc4c(cc3C(=O)N3Cc5ccccc5C[C@H]3CN3CCC[C@H](C)C3)CN(C(=O)Cc3ccc(OCCN5CCOCC5)cc3)CC4)n(C)c2C)c2ccc(O)cc2)cc(C#N)n1C]

### Step A: tert-butyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl-2-[2-[4-(2-morpholinoethoxy)phenyl]acetyl]-3,4-dihydro-1H-isoquinoline-7-carboxylate

Using General procedure 4c, without silyl deprotection step, starting from Preparation Va (193 mg, 1.2 eq.) as the appropriate carboxylic acid and the product from Step D of Preparation IX (400 mg, 0.58 mmol) as the appropriate amine, the title compound was obtained (400 mg, 74%).

¹H NMR (500 MHz, DMSO-d6) δ ppm 7.61-6.69 (m, 11H), 6.37/3.71 (t, 2H), 5.11/5.09 (s, 1H), 4.76/4.65 (s, 2H), 4.05/4.03 (t, 2H), 3.72 (s, 2H), 3.59/3.13 (s, 6H), 3.56 (m, 4H), 2.75/2.70 (t, 2H), 2.67 (t, 2H), 2.46 (m, 4H), 2.41/2.04 (s, 6H), 1.28/1.27 (s, 9H), 0.85/0.84 (s, 9H), 0.09/0.08 (s, 6H).

HRMS-ESI (m/z) [M+H]⁺ calcd for C₅₄H₆₉N₆O₇Si: 941.4991, found 941.4994.

### Step B: 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-2-[2-[4-(2-morpholinoethoxy)phenyl]acetyl]-3,4-dihydro-1H-isoquinoline-7-carboxylic acid

Using General procedure 3a starting from the product of Step A (400 mg, 0.41 mmol), the title compound was obtained (100 mg, 25%).

¹H NMR (400 MHz, DMSO-d6) δ ppm 12.64 (brs, 1H), 7.68-6.73 (m, 11H), 5.10 (s, 1H), 4.80-2.30 (m, 20H), 3.58/3.14 (s+s, 6H), 2.39/1.99 (s+s, 6H), 0.90 (s, 9H), 0.14 (s, 6H); ¹³C NMR (100 MHz, DMSO-d6) δ ppm 108.3, 33.2/31.5, 26.0, 11.9/10.1, −4.1.

HRMS-ESI (m/z) [M+H]⁺ calcd for C₅₀H₆₁N₆O₇Si: 885.4360, found 885.4357.

### Step C: N-(5-cyano-1,2-dimethyl-pyrrol-3-yl)-N-(4-hydroxyphenyl)-1,2-dimethyl-5-[7-[(3S)-3-[[(3S)-3-methyl-1-piperidyl]methyl]-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

Using General procedure 4c, starting from the product of Step B (80 mg, 0.09 mmol) as the appropriate carboxylic acid and (3S)-3-[[(2S)-2-methyl-1-piperidyl]methyl]-1,2,3,4-tetrahydroisoquinoline—which was obtained according to an analogous fashion to Example 125, Step A—(26 mg, 1.1 eq.) as the appropriate amine, the title compound was obtained (48 mg, 53%).

HRMS-ESI (m/z) [M+H]⁺ calcd for C₆₀H₆₉N₈O₆: 997.5334, found 997.5327.

### Example 154: N-(5-cyano-1,2-dimethylpyrrol-3-yl)-5-{2-[2-(4-{2-[4-(2,2-difluoroethyl)piperazin-1-yl]ethoxy}phenyl)acetyl]-7-{[(3S)-3-(pyrrolidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

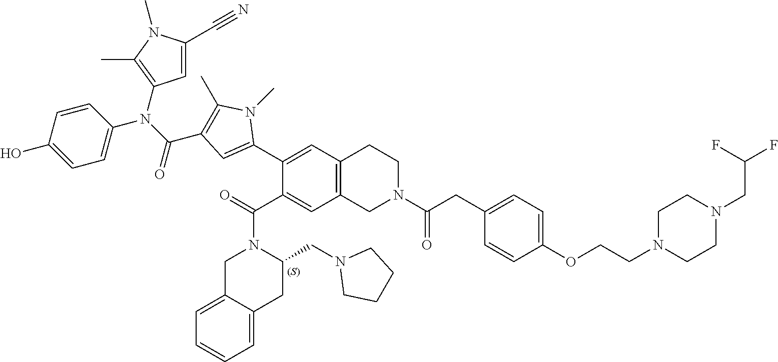

Using General procedure 4a and Preparation IVc (158 mg, 0.22 mmol, 1 eq.) as the appropriate amine and the product from Example 112, Step C (79 mg, 0.24 mmol, 1.1 eq.) as the appropriate carboxylic acid afforded the title product (100 mg, 0.1 mmol, 44%).

HRMS calcd for C₆₀H₆₇F₂N₉O₅: 1031.5233, found 1032.5309 [M+H]⁺

### Example 155: N-(5-cyano-1,2-dimethylpyrrol-3-yl)-5-(7-{[(3S)-3-{[ethyl(isopropyl)amino]methyl}-3,4-dihydro-1H-isoquinolin-2-yl]carbonyl}-2-(2-{2-fluoro-4-[2-(morpholin-4-yl)ethoxy]phenyl}acetyl)-3,4-dihydro-1H-isoquinolin-6-yl)-N-(4-hydroxyphenyl)-1,2-dimethylpyrrole-3-carboxamide

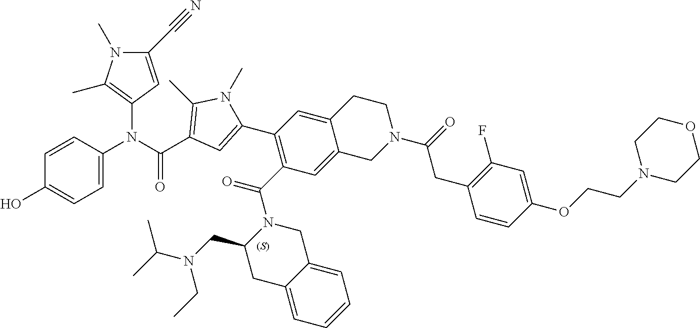

### Step A: tert-butyl (3S)-3-[ethyl(isopropyl)carbamoyl]-3,4-dihydro-1H-isoquinoline-2-carboxylate

Using General procedure 5 and Boc-(3S)-1,2,3,4-tetrahydroisoquinoline-3-carboxylic acid (500 mg, 1.8 mmol, 1 eq.) and N-ethylisopropylamine (0.23 mL, 1.89 mmol, 1.05 eq.) as the appropriate amine afforded the title product (534 mg, 1.54 mmol, 85%).

LRMS calcd for C₂₀H₃₀N₂O₃: 346.2, found: 347.4 [M+H]⁺

¹H NMR (400 MHz, DMSO-d6) δ ppm: 7.25-7.07 (m, 4H), 5.25-4.14 (m, 4H), 3.46-2.62 (m, 4H), 1.49-1.31 (m, 9H), 1.30-0.93 (m, 9H).

### Step B: ethyl(isopropyl)[(3S)-1,2,3,4-tetrahydroisoquinolin-3-ylmethyl]amine dihydrochloride

Using General procedure 6 and the product from Step A (534 mg, 1.54 mmol, 1 eq.) as the appropriate THIQ amide afforded the title product (470 mg, 100%).

LRMS calcd for C₁₅H₂₄N₂: 232.2, found 233.4 [M+H]⁺

¹H NMR (400 MHz, DMSO-d6) δ ppm: 10.69-10.02 (br m, 3H), 7.34-7.14 (m, 4H), 4.50-4.29 (m. 2H). 4.23-4.06 (m. 1H). 3.90-3.00 (m. 7H). 1.49-1.21 (m. 9H).

### Step C: N-(5-cyano-1,2-dimethylpyrrol-3-yl)-5-(7-{[(3S)-3-{[ethyl(isopropyl)amino]methyl}-3,4-dihydro-1H-isoquinolin-2-yl]carbonyl}-2-(2-{2-fluoro-4-[2-(morpholin-4-yl)ethoxy]phenyl}acetyl)-3,4-dihydro-1H-isoquinolin-6-yl)-N-(4-hydroxyphenyl)-1,2-dimethylpyrrole-3-carboxamide

Using General procedure 4a and Preparation IX (100 mg, 0.11 mmol, 1 eq.) as the appropriate carboxylic acid and the product from Step B (37 mg, 0.12 mmol, 1.1 eq.) as the appropriate amine afforded an intermediate which was treated according to General procedure 3c to afford the title product (33.4 mg, 0.03 mmol, 30%).

HRMS calcd for C₅₉H₆₇FN₈O₆: 1002.5168, found 1003.5177 [M+H]⁺

### Example 156: N-(5-cyano-1,2-dimethylpyrrol-3-yl)-5-[2-(2-{2-fluoro-4-[2-(morpholin-4-yl)ethoxy]phenyl}acetyl)-7-{[(3S)-3-{[(3R)-3-methylpyrrolidin-1-yl]methyl}-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

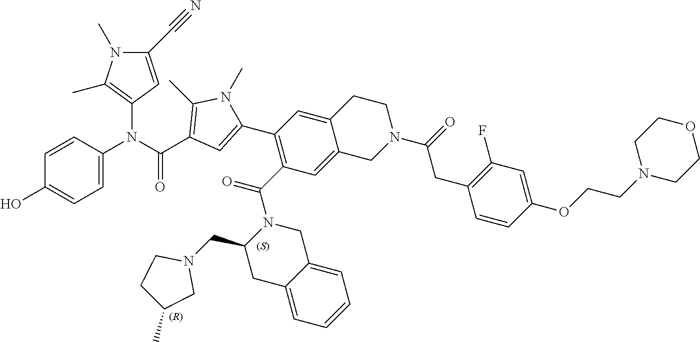

### Step A: tert-butyl (3S)-3-{[(3R)-3-methylpyrrolidin-1-yl]carbonyl}-3,4-dihydro-1H-isoquinoline-2-carboxylate

Using General procedure 5 and Boc-(3S)-1,2,3,4-tetrahydroisoquinoline-3-carboxylic acid (500 mg, 1.8 mmol, 1 eq.) and (R)-3-methyl-pyrrolidine hydrochloride (241 mg, 1.98 mmol, 1.1 eq.) as the appropriate amine afforded the title product (380 mg, 1.1 mmol, 61%).

LRMS calcd for C₂₀H₂₈N₂O₃: 344.2, found 345.4 [M+H]⁺

¹H NMR (400 MHz, DMSO-d6) δ ppm: 7.27-7.08 (m, 4H), 4.96-4.17 (m, 3H), 3.78-2.65 (m, 6H), 2.43-1.85 (m, 2H), 1.73-1.46 (m, 10H), 1.08-0.94 (m, 3H).

### Step B: (3S)-3-{[(3R)-3-methylpyrrolidin-1-yl]methyl}-1,2,3,4-tetrahydroisoquinoline dihydrochloride

Using General procedure 6 and the product from Step A (529 mg, 1.54 mmol, 1 eq.) as the appropriate THIQ amide afforded the title product (465 mg, 1.53 mmol, 100%).

LRMS calcd for C₁₅H₂₂N₂: 230.2, found 231.2 [M+H]⁺

¹H NMR (400 MHz, DMSO-d6) δ ppm: 11.19-10.79 (br m, 1H), 10.29-9.72 (br m, 2H), 7.35-7.12 (m, 4H), 4.51-4.34 (m, 2H), 4.11-3.97 (m, 1H), 3.88-2.94 (m, 8H), 2.81-2.05 (m, 2H), 1.74-1.41 (m, 1H), 1.08 (d, J=6.7 Hz, 3H).

### Step C: N-(5-cyano-1,2-dimethylpyrrol-3-yl)-5-[2-(2-{2-fluoro-4-[2-(morpholin-4-yl)ethoxy]phenyl]acetyl)-7-{[(3S)-3-{[(3R)-3-methylpyrrolidin-1-yl]methyl]-3,4-dihydro-1H-isoquinolin-2-yl]carbonyl]-3,4-dihydro-1H-isoquinolin-6-yl]-N-(4-hydroxyphenyl)-1,2-dimethylpyrrole-3-carboxamide

Using General procedure 4a and Preparation IX (100 mg, 0.11 mmol, 1 eq.) as the appropriate carboxylic acid and the product from Step B (37 mg, 0.12 mmol, 1.1 eq.) as the appropriate amine afforded an intermediate which was treated according to General procedure 3c to afford the title product (17.9 mg, 0.02 mmol, 18%).

HRMS calcd for C₅₉H₆₅FN₈O₆: 1000.5011, found 1001.5085 [M+H]⁺

### Example 157: N-(5-cyano-1,2-dimethylpyrrol-3-yl)-5-[2-(2-{2-fluoro-4-[2-(morpholin-4-yl)ethoxy]phenyl}acetyl)-7-{[(3S)-3-{[(3S)-3-methylpyrrolidin-1-yl]methyl}-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

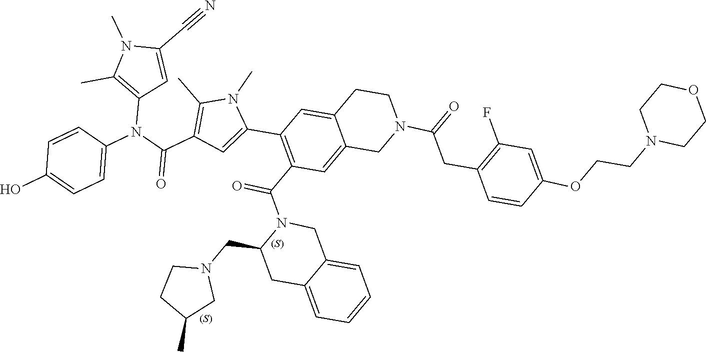

### Step A: tert-butyl (3S)-3-{[(3S)-3-methylpyrrolidin-1-yl]carbonyl}-3,4-dihydro-1H-isoquinoline-2-carboxylate

Using General procedure 5 and Boc-(3S)-1,2,3,4-tetrahydroisoquinoline-3-carboxylic acid (350 mg, 1.26 mmol, 1 eq.) and (S)-3-methyl-pyrrolidine hydrochloride (153 mg, 1.26 mmol, 1.1 eq.) as the appropriate amine afforded the title product (435 mg, quant.).

LRMS calcd for C₂₀H₂₈N₂O₃: 344.2, found 345.4 [M+H]⁺

¹H NMR (400 MHz, DMSO-d6) δ ppm: 7.28-7.08 (m, 4H), 4.95-4.17 (m, 3H), 3.79-2.70 (m, 6H), 2.40-1.86 (m, 2H), 1.65-1.24 (m, 10H), 1.11-0.93 (m, 3H).

### Step B: (3S)-3-{[(3S)-3-methylpyrrolidin-1-yl]methyl}-1,2,3,4-tetrahydroisoquinoline dihydrochloride

Using General procedure 6 and the product from Step A (435 mg, 1.26 mmol, 1 eq.) as the appropriate THIQ amide afforded the title product (383 mg, quant.).

LRMS calcd for C₁₅H₂₂N₂: 230.2, found 231.2 [M+H]⁺

¹H NMR (400 MHz, DMSO-d6) δ ppm: 11.17-10.81 (br m, 1H), 10.30-9.79 (br m, 2H), 7.33-7.17 (m, 4H), 4.50-4.34 (m, 2H), 4.12-2.94 (m, 9H), 2.81-2.03 (m, 2H), 1.75-1.47 (m, 1H), 1.08 (d, J=6.7 Hz, 3H).

### Step C: N-(5-cyano-1,2-dimethylpyrrol-3-yl)-5-[2-(2-{2-fluoro-4-[2-(morpholin-4-yl)ethoxy]phenyl]acetyl)-7-{[(3S)-3-{[(3S)-3-methylpyrrolidin-1-yl]methyl]-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and Preparation IX (123 mg, 0.14 mmol, 1 eq.) as the appropriate carboxylic acid and the product from Step B (45 mg, 0.15 mmol, 1.1 eq.) as the appropriate amine afforded an intermediate which was treated according to General procedure 3c to afford the title product (17.6 mg, 0.02 mmol, 11%).

HRMS calcd for C₅₉H₆₅FN₈O₆: 1000.5011, found 1001.5088 [M+H]⁺

### Example 158: N-[5-cyano-1-(difluoromethyl)-2-methylpyrrol-3-yl]-5-[2-(2-{2-fluoro-4-[2-(4-fluoropiperidin-1-yl)ethoxyphenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

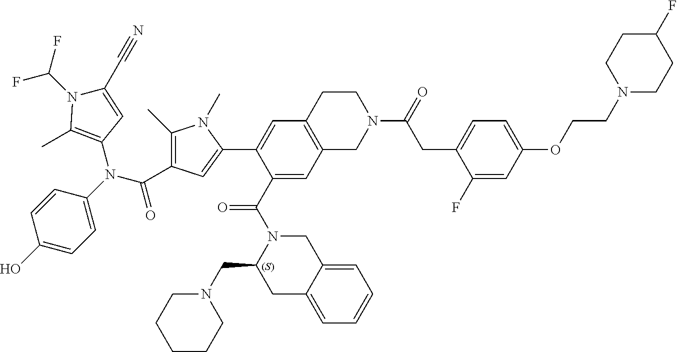

### Step A: methyl 2-[4-(2-chloroethoxy)-2-fluorophenyl]acetate

Using General procedure 7b and the product from Preparation Vb, Step A (1.5 g, 8.14 mmol, 1 eq.) as the appropriate phenol and 2-chloroethanol (0.82 mL, 12.22 mmol, 1.5 eq.) as the appropriate alcohol afforded the title product (1.48 g, 5.98 mmol, 73%).

LRMS calcd for C₁H₁₂ClFO₃: 246.0, found 247.2 [M+H]⁺

¹H NMR (400 MHz, DMSO-d6) δ ppm: 7.25 (t, J=8.7 Hz, 1H), 6.86 (dd, J=11.9, 2.5 Hz, 1H), 6.78 (ddd, J=8.5, 2.5, 0.7 Hz, 1H), 4.30-4.23 (m, 2H), 3.97-3.91 (m, 2H), 3.67-3.63 (m, 2H), 3.61 (s, 3H).

### Step B: methyl 2-{2-fluoro-4-[2-(4-fluoropiperidin-1-yl)ethoxy]phenyl}acetate

To a solution of the product from Step A (480 mg, 1.95 mmol, 1 eq.) and 4-fluoropiperidine (0.22 mL, 2.14 mmol, 1.1 eq) in MeCN (32 mL) were added K₂CO₃ (1.08 g, 7.78 mmol, 4 eq.) and NaI (583 mg, 3.89 mmol, 2 eq.). The mixture was heated at 80° C. under N₂ with vigorous stirring for 18 h. The reaction mixture was allowed to cool to rt and filtered, washing with EtOAc. The filtrate was concentrated under reduced pressure and purified by automated flash chromatography eluting with a gradient of 0-100% EtOAc in heptane to afford the title product (179 mg, 0.57 mmol, 29%).

LRMS calcd for C₁₆H₂₁F₂NO₃: 313.1, found 314.2 [M+H]⁺

¹H NMR (400 MHz, DMSO-d6) δ ppm: 7.22 (t, J=8.7 Hz, 1H), 6.82 (dd, J=12.1, 2.5 Hz, 1H), 6.76-6.72 (m, 1H), 4.77-4.56 (m, 1H), 4.06 (t, J=5.8 Hz, 2H), 3.64-3.62 (m, 2H), 3.61 (s, 3H), 2.68 (t, J=5.8 Hz, 2H), 2.66-2.57 (m, 2H), 2.43-2.34 (m, 2H), 1.93-1.77 (m, 2H), 1.75-1.62 (m, 2H).

### Step C: 2-{2-fluoro-4-[2-(4-fluoropiperidin-1-yl)ethoxy]phenyl}acetic acid

Using General procedure 8 and the product from Step B (179 mg, 0.57 mmol, 1 eq.) as the appropriate ester afforded the title product (116 mg, 68%).

LRMS calcd for C₁₅H₁₉F₂NO₃: 299.1, found 300.4 [M+H]⁺

¹H NMR (400 MHz, DMSO-d6) δ ppm: 7.16-7.09 (m, 1H), 6.68-6.58 (m, 2H), 4.77-4.55 (m, 1H), 4.02 (t, J=5.9 Hz, 2H), 3.10-3.06 (m, 2H), 2.67 (t, J=5.9 Hz, 2H), 2.65-2.57 (m, 2H), 2.43-2.34 (m, 2H), 1.93-1.77 (m, 2H), 1.75-1.62 (m, 2H).

### Step D: N-[5-cyano-1-(difluoromethyl)-2-methylpyrrol-3-yl]-5-[2-(2-{2-fluoro-4-[2-(4-fluoropiperidin-1-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl-N-(4-hydroxyphenyl)-1,2-dimethylpyrrole-3-carboxamide

Using General procedure 4a and the product from Example 77, Step C (181 mg, 0.19 mmol, 1 eq.) as the appropriate amine and the product from Step C (64 mg, 0.21 mmol, 1.1 eq.) as the appropriate carboxylic acid afforded an intermediate which was treated according to General procedure 3b to afford the title product (43.6 mg, 0.04 mmol, 21%).

HRMS calcd for C₆₀H₆₄F₄N₈O₅: 1052.4936, found 1053.5009 [M+H]⁺

### Example 159: N-[5-cyano-1-(difluoromethyl)-2-methylpyrrol-3-yl]-5-{2-[2-(2-fluoro-4-{2-[4-(2-fluoroethyl)piperazin-1-yl]ethoxy}phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

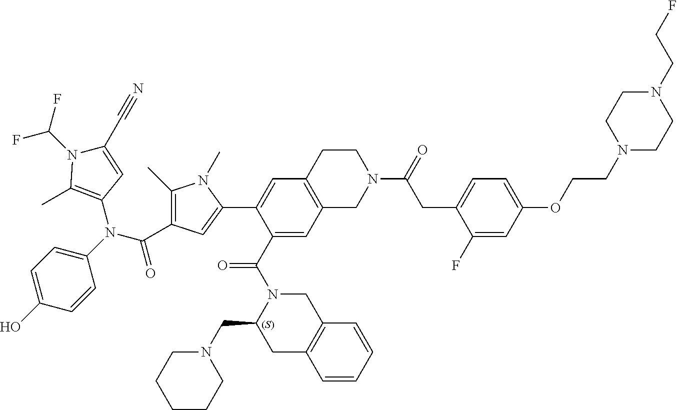

### Step A: N-[5-cyano-1-(difluoromethyl)-2-methylpyrrol-3-yl]-N-(4-hydroxyphenyl)-1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxamide

Using General procedure 3b and Example 77, Step B (397 mg, 0.39 mmol, 1 eq.) as the appropriate silyl-derivative and heating at 50° C. afforded the title product (216 mg, 0.28 mmol, 72%).

LRMS calcd for C₄₅H₄₇F₂N₇O₃: 771.4, found 772.6 [M+H]⁺

### Step B: N-[5-cyano-1-(difluoromethyl)-2-methylpyrrol-3-yl]-5-{2-[2-(2-fluoro-4-{2-[4-(2-fluoroethyl)piperazin-1-yl]ethoxy]phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

Using General procedure 4a and the product from Step A (216 mg, 0.28 mmol, 1 eq.) as the appropriate amine and the product of Step A of Example 117 (101 mg, 0.31 mmol, 1.1 eq.) as the appropriate carboxylic acid afforded the title product (124.6 mg, 0.12 mmol, 41%).

HRMS calcd for C₆₁H₆₇F₄N₉O₅: 1081.5201, found 1082.5278 [M+H]⁺

### Example 160: N-[5-cyano-1-(difluoromethyl)-2-methylpyrrol-3-yl]-5-[2-(2-{2-fluoro-4-[2-(4-methylpiperazin-1-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

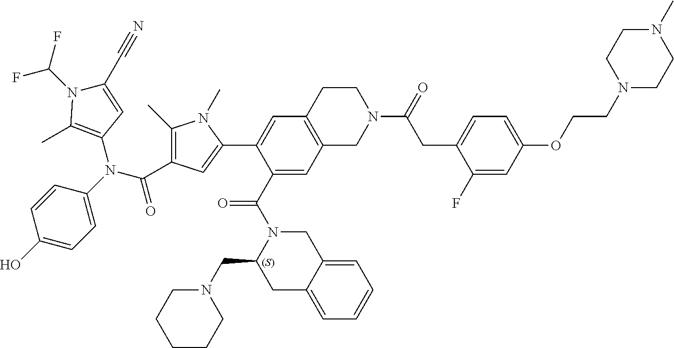

### Step A: methyl 2-{2-fluoro-4-[2-(4-methylpiperazin-1-yl)ethoxy]phenyl}acetate

Using General procedure 7b and the product from Preparation Vb, Step A (220 mg, 1.19 mmol, 1 eq.) as the appropriate phenol and 1-(2-hydroxyethyl)-4-methylpiperazine (258 mg, 1.79 mmol, 1.5 eq.) as the appropriate alcohol afforded the title product (343 mg, 1.11 mmol, 93%).

LRMS calcd for C₁₆H₂₃FN₂O₃: 310.2, found 311.2 [M+H]⁺

¹H NMR (400 MHz, DMSO-d6) δ ppm: 7.25-7.18 (m, 1H), 6.82 (dd, J=12.0, 2.5 Hz, 1H), 6.76-6.72 (m, 1H), 4.06 (t, J=5.8 Hz, 2H), 3.64-3.62 (m, 2H), 3.61 (s, 3H), 2.66 (t, J=5.8 Hz, 2H), 2.56-2.19 (br m, 8H), 2.14 (s, 3H).

### Step B: 2-{2-fluoro-4-[2-(4-methylpiperazin-1-yl)ethoxy]phenyl}acetic acid

Using General procedure 8 and the product from Step A (343 mg, 1.11 mmol, 1 eq.) as the appropriate ester afforded the title product (249 mg, 0.84 mmol, 76%).

LRMS calcd for C₁₅H₂₁FN₂O₃: 296.2, found: 297.2 [M+H]⁺

¹H NMR (400 MHz, DMSO-d6) δ ppm: 7.17 (t, J=8.8 Hz, 1H), 6.76 (dd, J=11.9, 2.5 Hz, 1H), 6.72-6.67 (m, 1H), 4.05 (t, J=5.8 Hz, 2H), 3.44-3.38 (m, 2H), 2.65 (t, J=5.8 Hz, 2H), 2.56-2.18 (m, 8H), 2.14 (s, 3H).

### Step C: N-[5-cyano-1-(difluoromethyl)-2-methylpyrrol-3-yl]-5-[2-(2-{2-fluoro-4-[2-(4-methylpiperazin-1-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl-N-(4-hydroxyphenyl)-1,2-dimethylpyrrole-3-carboxamide

Using General procedure 4a and Example 77, Step C (181 mg, 0.19 mmol, 1 eq.) as the appropriate amine and the product from Step B (64 mg, 0.21 mmol, 1.1 eq.) as the appropriate carboxylic acid afforded an intermediate which was treated according to General procedure 3b to afford the title product (51.6 mg, 0.05 mmol, 25%).

HRMS calcd for C₆₀H₆₆F₃N₉O₅: 1049.5139, found 1050.5215 [M+H]⁺

### Example 161: 5-[2-[2-[2-chloro-4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(5-cyano-1,2-dimethyl-pyrrol-3-yl)-N-(4-hydroxyphenyl)-1,2-dimethyl-pyrrole-3-carboxamide

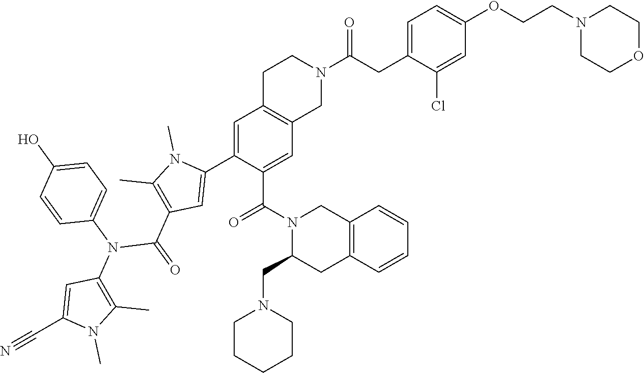 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCOCC5)cc3Cl)CC4)n(C)c2C)c2ccc(O)cc2)cc(C#N)n1C]

Using General Procedure 4c, starting from the product obtained in Example 46, Step A (76 mg, 1.8 eq.) as the appropriate acid and Preparation VIIa (90 mg. 0.106 mmol) as the appropriate amine, afforded the title product (50.4 mg, 47%).

HRMS-ESI (m/z) [M+H]⁺ calcd for C₅₉H₆₆C₁N₈O: 1017.4788, found 1017.4783.

### Example 162: N-[5-cyano-1-(difluoromethyl)-2-methyl-pyrrol-3-yl]-5-[2-[2-[2-fluoro-4-[2-((2R or S)-2-methylmorpholin-4-yl)ethoxy]phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereomer 2)

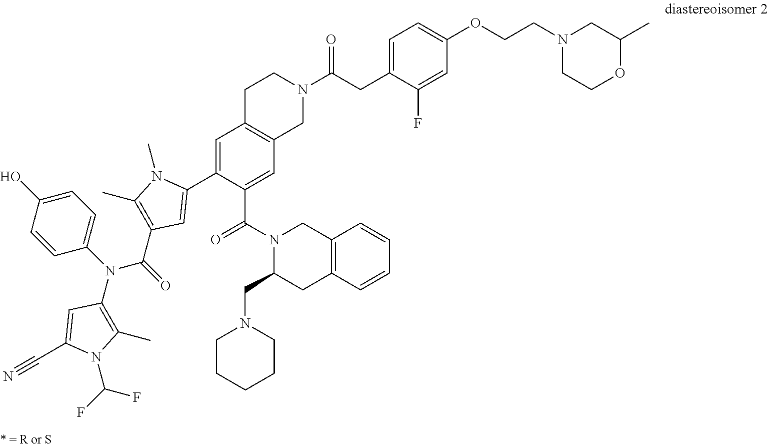 [SMILES: Cc1c(C(=O)N(c2ccc(O)cc2)c2cc(C#N)n(C(F)F)c2C)cc(-c2cc3c(cc2C(=O)N2Cc4ccccc4C[C@H]2CN2CCCCC2)CN(C(=O)Cc2ccc(OCCN4CCOC(C)C4)cc2F)CC3)n1C]

### Step A: 4-(4-benzyloxyanilino)-1-(difluoromethyl)-5-methyl-pyrrole-2-carbonitrile

Using General procedure 1b, starting from 4-bromo-]-(difluoromethyl)-5-methyl-pyrrole-2-carbonitrile (1.0 g, 4.25 mmol) as the appropriate aryl bromide and 4-benzyloxyaniline (1.1 g, 1.3 eq.), afforded the title product (1.01 g, 67%).

¹H NMR (400 MHz, DMSO-d6) δ ppm 7.79 (t, 1H), 7.45-7.27 (m, 5H), 7.15 (s, 1H), 7.11 (s, 1H), 6.83 (m, 2H), 6.61 (m, 2H), 4.98 (s, 2H), 2.24 (s, 3H); ¹³C NMR (100 MHz, DMSO-d6) δ ppm 128.4, 127.3, 121.1, 116.2, 115, 112.9, 109.7, 97.7, 70.1, 9.8.

HRMS-ESI (m/z) [M+H]⁺ calcd for C₂₀H₁₈F₂N₃O: 354.1412, found 354.1413.

### Step B: 9H-fluoren-9-ylmethyl 6-[4-[(4-benzyloxyphenyl)-[5-cyano-1-(difluoromethyl)-2-methyl-pyrrol-3-yl]carbamoyl]-1,5-dimethyl-pyrrol-2-yl]-7-[(3S)-3-(1-piperidylmethyl)-3,4-dihydro-1H-isoquinoline-2-carbonyl]-3,4-dihydro-1H-isoquinoline-2-carboxylate

Using General procedure 2b, starting from the compound obtained in Step B of Preparation VIIa (500 mg, 0.67 mmol) as the appropriate acid and the product of Step A (280 mg, 1.2 eq.) as the appropriate aniline, the title compound was obtained (600 mg, 83%).

¹H NMR (500 MHz, DMSO-d6) δ ppm 8.02-6.71 (m, 24H), 7.81 (t, 1H), 5.53-5.02 (s, 1H), 5.45-1.16 (m, 28H), 3.53-2.95 (s, 3H), 2.44-1.89 (s, 6H).

HRMS-ESI (m/z) [M+H]⁺ calcd for C₆₇H₆₄F₂N₇O: 1084.4932, found 1084.4929.

### Step C. N-(4-benzyloxyphenyl)-N-[5-cyano-1-(difluoromethyl)-2-methyl-pyrrol-3-yl]-5-[2-[2-[2-fluoro-4-[2-((2R or S)-2-methylmorpholin-4-yl)ethoxy]phenyl]acetyl]-7-[(3S)-3-(1-piperidylmethyl)-3,4-dihydro-1H-isoquinoline-2-carbonyl]-3,4-dihydro-1H-isoquinolin-6-yl]-1,2-dimethyl-pyrrole-3-carboxamide

To a solution of the product of Step B (155 mg, 0.143 mmol) in DMF (7 mL/mmol) was added DBU (10 mg, 0.5 eq.) and the mixture was stirred until the Fmoc protecting group was completely removed. Then, using General Procedure 4c without the silyl deprotection step, starting from the compound obtained in Step A of Example 109 (55 mg, 1.3 eq.) as the appropriate acid and the mixture described above containing the appropriate amine, afforded the title product (100 mg, 61%).

¹H NMR (500 MHz, DMSO-d6) δ ppm 7.79 (t, 1H), 7.46-6.68 (m, 19H), 5.7-5.1 (s, 1H), 5.12-1.70 (m, 13H), 5.11-4.88 (s, 2H), 4.06 (t, 2H), 3.71/3.47 (m+m, 2H), 3.49 (m, 1H), 3.35-3.06 (s, 3H), 2.81/1.78 (m+m, 2H), 2.73/2.07 (m+m, 2H), 2.65 (t, 2H), 2.55-1.60 (m, 6H), 2.43-1.91 (s, 6H), 1.58-1.18 (m, 6H), 1.07-1.00 (d, 3H).

HRMS-ESI (m/z) [M+H]⁺ calcd for C₆₇H₇₂F₃N₈O: 1141.5521, found 1141.5514.

### Step D: N-[5-cyano-1-(difluoromethyl)-2-methyl-pyrrol-3-yl]-5-[2-[2-[2-fluoro-4-[2-((2R or S)-2-methylmorpholin-4-yl)ethoxy]phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

The compound obtained in Step C (100 mg, 0.088 mol) was dissolved in methanol (57 mL/mmol), the flask was evacuated and backfilled with nitrogen, then Pd catalyst (10% on activated carbon) was added (15 mg, 0.16 eq.) under nitrogen, the mixture was evacuated and the flask was stirred vigorously under an atmosphere of hydrogen until complete conversion was observed. Then, the mixture was filtered through a pad of Celite, the pad was washed with methanol, the filtrate was concentrated and the product was purified via preparative reversed phase chromatography to afford the title product (59 mg, 64%).

HRMS-ESI (m/z) [M+H]⁺ calcd for C₆₀H₆₆F₃N₈O: 1051.5052, found 1051.5048.

### Example 163: N-[5-cyano-1-(difluoromethyl)-2-methyl-pyrrol-3-yl]-5-[2-[2-[4-[2-[cyclopropyl(methyl)amino]ethoxy]phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

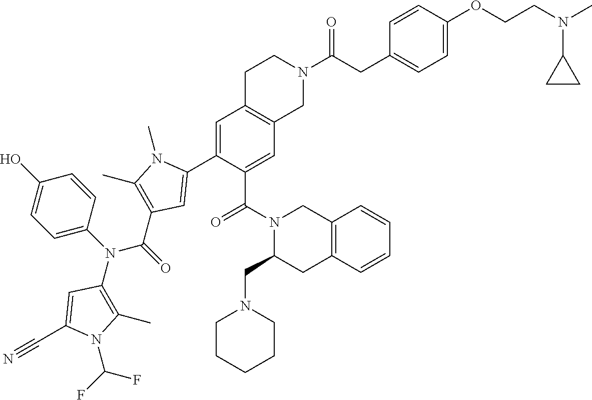 [SMILES: Cc1c(C(=O)N(c2ccc(O)cc2)c2cc(C#N)n(C(F)F)c2C)cc(-c2cc3c(cc2C(=O)N2Cc4ccccc4C[C@H]2CN2CCCCC2)CN(C(=O)Cc2ccc(OCCN(C)C4CC4)cc2)CC3)n1C]

### Step A: N-(4-benzyloxyphenyl)-N-[5-cyano-1-(difluoromethyl)-2-methyl-pyrrol-3-yl]-5-[2-[2-[4-[2-[cyclopropyl(methyl)amino]ethoxy]phenyl]acetyl]-7-[(3S)-3-(1-piperidylmethyl)-3,4-dihydro-1H-isoquinoline-2-carbonyl]-3,4-dihydro-1H-isoquinolin-6-yl-1,2-dimethyl-pyrrole-3-carboxamide

To a solution of the product of Step B of Example 162 (155 mg, 0.143 mmol) in DMF (7 mL/mmol) was added DBU (10 mg, 0.5 eq.) and the mixture was stirred until the Fmoc protecting group was completely removed. Then, using General Procedure 4c without the silyl deprotection step, starting from the compound obtained in Step G of Example 35 (60 mg, 1.7 eq.) as the appropriate acid and the mixture described above containing the appropriate amine, afforded the title product (110 mg, 70%).

¹H NMR (500 MHz, DMSO-d6) δ ppm 7.80 (t, 1H), 7.46-6.73 (m, 20H), 5.70-5.08 (s, 1H), 5.15-1.68 (m, 13H), 5.10-4.88 (s, 2H), 4.03 (t, H), 3.34-3.03 (s, 3H), 2.83 (t, 2H), 2.50-1.60 (m, 6H), 2.42-1.90 (s, 6H), 2.32 (s, 3H), 1.72 (m, 1H), 1.58-1.16 (m, 6H), 0.84-0.22 (m, 4H).

HRMS-ESI (m/z) [M+H]⁺ calcd for C₆₆H₇₁F₂N₈O₅:1093.5510, found 1093.5516.

### Step B: N-[5-cyano-1-(difluoromethyl)-2-methyl-pyrrol-3-yl]-5-[2-[2-[4-[2-[cyclopropyl(methyl)amino]ethoxy]phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

The compound obtained in Step A (110 mg, 0.10 mol) was dissolved in methanol (57 mL/mmol), the flask was evacuated and backfilled with nitrogen, then Pd catalyst (10% on activated carbon) was added (15 mg) under nitrogen, the mixture was evacuated and the flask was stirred vigorously under an atmosphere of hydrogen until complete conversion was observed. Then, the mixture was filtered through a pad of Celite, the pad was washed with methanol, the filtrate was concentrated and the product was purified via preparative reversed phase chromatography to afford the title product (44 mg, 44%).

HRMS-ESI (m/z) [M+H]⁺ calcd for C₅₉H₆₅F₂N₈O₅: 1003.5040, found 1003.5042.

### Example 164: 5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(5-fluoro-6-methoxypyridin-3-yl)-N-(4-hydroxyphenyl)-1,2-dimethylpyrrole-3-carboxamide

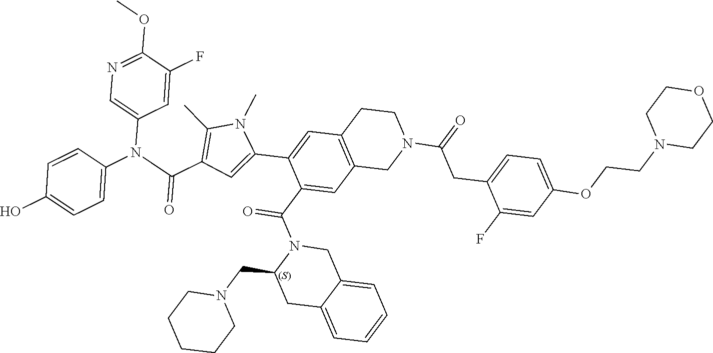

### Step A: 5-fluoro-6-methoxy-N-{4-[(triisopropylsilyl)oxy]phenyl}pyridin-3-amine

Using General procedure 1a and 5-bromo-3-fluoro-2-methoxypyridine (415 mg, 2.01 mmol, 1 eq.) as the appropriate aryl bromide and Preparation IIb (588 mg, 2.22 mmol, 1.1 eq.) as the appropriate aniline afforded the title product (450 mg, 1.15 mmol, 57%).

LRMS calcd for C₂₁H₃₁FN₂O₂Si: 390.2, found 391.4 [M+H]⁺

¹H NMR (400 MHz, DMSO-d6) δ ppm: 7.87 (s, 1H), 7.66 (d, J=2.4 Hz, 1H), 7.28 (dd, J=12.4, 2.4 Hz, 1H), 6.97-6.88 (m, 2H), 6.82-6.74 (m, 2H), 3.86 (s, 3H), 1.29-1.14 (m, 3H), 1.06 (d, J=7.2 Hz, 18H).

### Step B: tert-butyl 6-{4-[(5-fluoro-6-methoxypyridin-3-yl)({4-[(triisopropylsilyl)oxy]phenyl})carbamoyl]-1,5-dimethylpyrrol-2-yl}-7-f[(3S)-3-(piperidin-1-ylmethyl)-3,4-dihydro-1H-isoquinolin-2-yl]carbonyl}-3,4-dihydro-1H-isoquinoline-2-carboxylate

Using General procedure 2a and Preparation IIb (300 mg, 0.48 mmol, 1 eq.) as the appropriate acid and the product from Step A (280 mg, 0.72 mmol, 1.5 eq.) as the appropriate aniline afforded the title product (200.7 mg, 0.2 mmol, 42%).

LRMS calcd for C₅₈H₇₅FN₆O₆Si: 998.6, found 999.8 [M+H]⁺

### Step C: N-(5-fluoro-6-methoxypyridin-3-yl)-N-(4-hydroxyphenyl)-1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxamide

Using General procedure 3b and the product from Step B (200 mg, 0.2 mmol, 1 eq.) as the appropriate silyl-derivative afforded the title product (101 mg, 0.14 mmol, 68%).

LRMS calcd for C₄₄H₄₇FN₆O₄: 742.4, found 743.4 [M+H]⁺

### Step D: 5-[2-(2-{2-fluoro-4-[2-(morpholin-4-yl)ethoxy]phenyl}acetyl)-7-f[(3S)-3-(piperidin-1-ylmethyl)-3,4-dihydro-1H-isoquinolin-2-yl]carbonyl}-3,4-dihydro-1H-isoquinolin-6-yl]-N-(5-fluoro-6-methoxypyridin-3-yl)-N-(4-hydroxyphenyl)-1,2-dimethylpyrrole-3-carboxamide

Using General procedure 4a and the product from Step C (101 mg, 0.14 mmol, 1 eq.) as the appropriate amine and Preparation Vb (46 mg, 0.16 mmol, 1.2 eq.) as the appropriate carboxylic acid afforded the title product (33.9 mg, 0.03 mmol, 25%).

HRMS calcd for C₅₈H₆₃F₂N₇O₇: 1007.4757, found 1008.4828 [M+H]⁺

### Example 165: 5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-N-(6-methoxypyridin-3-yl)-1,2-dimethylpyrrole-3-carboxamide

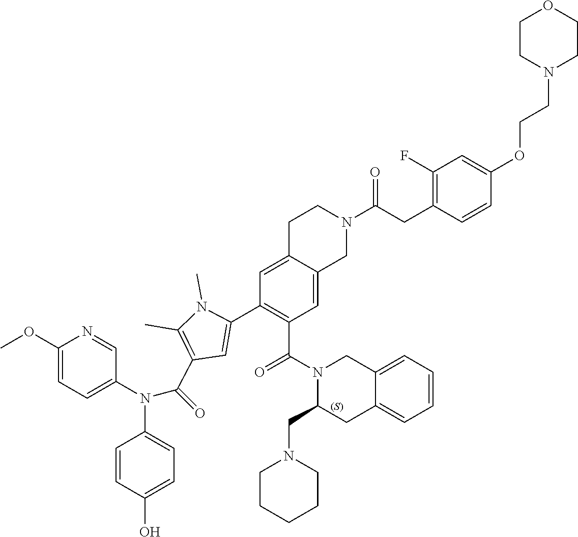

### Step A: 6-methoxy-N-{4-[(triisopropylsilyl)oxy]phenyl}pyridin-3-amine

Using General procedure 1a and 5-bromo-2-methoxypyridine (0.69 mL, 5.32 mmol, 1 eq.) as the appropriate aryl bromide and Preparation IIIb (1.48 g, 5.58 mmol, 1.05 eq.) as the appropriate aniline afforded the title product (774 mg, 2.08 mmol, 39%).

LRMS calcd for C₂₁H₃₂N₂O₂Si: 372.2, found 373.2 [M+H]⁺

¹H NMR (400 MHz, DMSO-d6) δ ppm: 7.85 (dd, J=2.9, 0.7 Hz, 1H), 7.64 (s, 1H), 7.39 (dd, J=8.8, 2.9 Hz, 1H), 6.87-6.81 (m, 2H), 6.77-6.69 (m, 3H), 3.78 (s, 3H), 1.29-1.14 (m, 3H), 1.06 (d, J=7.3 Hz, 18H).

### Step B: tert-butyl 6-{4-[(6-methoxypyridin-3-yl)([4-[(triisopropylsilyl)oxy]phenyl})carbamoyl]-1,5-dimethylpyrrol-2-yl}-7-f[(3S)-3-(piperidin-1-ylmethyl)-3,4-dihydro-1H-isoquinolin-2-yl]carbonyl}-3,4-dihydro-1H-isoquinoline-2-carboxylate

Using General procedure 2a and Preparation IIb (310 mg, 0.49 mmol, 1 eq.) as the appropriate acid and the product from Step A (295 mg, 0.79 mmol, 1.6 eq.) as the appropriate aniline afforded the title product (202 mg, 0.21 mmol, 42%).

LRMS calcd for C₅₈H₇₆N₆O₆Si: 980.6, found 981.8 [M+H]⁺

### Step C. N-(4-hydroxyphenyl)-N-(6-methoxypyridin-3-yl)-1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxamide

Using General procedure 3b and the product from Step B (201 mg, 0.2 mmol, 1 eq.) as the appropriate silyl-derivative afforded the title product (30 mg, 0.04 mmol, 20%).

LRMS calcd for C₄₄H₄₈N₆O₄: 724.4, found: 725.4 [M+H]⁺

### Step D: 5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-N-(6-methoxypyridin-3-yl)-1,2-dimethylpyrrole-3-carboxamide

Using General procedure 4a and the product from Step C (30 mg, 0.04 mmol, 1 eq) as the appropriate amine and Preparation Vb (12 mg, 0.04 mmol, 1.05 eq.) as the appropriate carboxylic acid afforded the title product (7.9 mg, 0.01 mmol, 19%).

HRMS calcd for C₅₈H₆₄FN₇O₇: 989.4851, found: 990.4924 [M+H]⁺

### Example 166: N-(5-cyano-1,2-dimethylpyrrol-3-yl)-5-{2-[2-(2-fluoro-4-{2-[(3R)-3-methylmorpholin-4-yl]ethoxy}phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

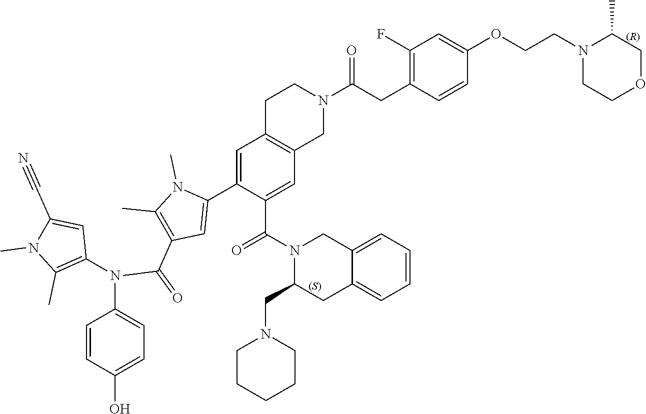

### Step A: methyl 2-(2-fluoro-4-{2-[(3R)-3-methylmorpholin-4-yl]ethoxy}phenyl)acetate

To a mixture of (R)-3-methylmorpholine (349 μL, 3.1 mmol, 1.2 eq.), Example 158, Step A (638 mg, 2.59 mmol, 1 eq.) and K₂CO₃ (715 mg, 5.17 mmol, 2 eq.) in MeCN (40 mL) was added KI (86 mg, 0.52 mmol, 0.2 eq.) and then the mixture was refluxed for 48 h. The mixture was allowed to cool to rt and the solids filtered and washed with EtOAc. The combined filtrate was evaporated and partitioned between EtOAc and water. The phases were separated, and the organic phase was washed with water and brine, dried over MgSO₄ and concentrated in vacuo. Purification by automated flash chromatography eluting with a gradient of 0-100% EtOAc in heptane afforded the title product (411 mg, 1.32 mmol, 51%).

LRMS calcd for C₁₆H₂₂FNO₄: 311.2, found 312.2 [M+H]⁺

¹H NMR (400 MHz, DMSO-d6) δ ppm: 7.22 (t, J=8.7 Hz, 1H), 6.82 (dd, J=12.1, 2.5 Hz, 1H), 6.78-6.71 (m, 1H), 4.06 (t, J=5.9 Hz, 2H), 3.71-3.55 (m, 7H), 3.51-3.43 (m, 1H), 3.12-2.97 (m, 2H), 2.81-2.75 (m, 1H), 2.63-2.54 (m, 1H), 2.49-2.32 (m, 2H), 0.92 (d, J=6.3 Hz, 3H).

### Step B: 2-(2-fluoro-4-{2-[(3R)-3-methylmorpholin-4-yl]ethoxy]phenyl)acetic acid

Using General procedure 8 and the product from Step A (409 mg, 1.31 mmol, 1 eq.) as the appropriate ester afforded the title product (158 mg, 0.53 mmol, 40%).

LRMS calcd for C₁₅H₂₀FNO₄: 297.1, found 298.2 [M+H]⁺

¹H NMR (400 MHz, DMSO-d6) δ ppm: 12.36 (br s, 1H), 11.46-11.00 (m, 1H), 7.27 (t, J=8.7 Hz, 1H), 6.94-6.86 (m, 1H), 6.85-6.76 (m, 1H), 4.51-4.34 (m, 2H), 4.02-3.19 (m, 11H), 1.34/1.28 (d, 3H).

### Step C: N-(5-cyano-1,2-dimethylpyrrol-3-yl)-5-{2-[2-(2-fluoro-4-{2-[(3R)-3-methylmorpholin-4-yl]ethoxy]phenyl)acetyl]-7-{[(3S)-3-(piperidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide

Using General procedure 4a and Preparation VIIa (149 mg, 0.18 mmol, 1 eq.) as the appropriate amine and the product from Step B (63 mg, 0.21 mmol, 1.2 eq.) as the appropriate carboxylic acid afforded an intermediate which was treated according to General procedure 3b to afford the title product (42 mg, 0.04 mmol, 24%).

HRMS calcd for C₆₀H₆₇FN₈O₆: 1014.5168, found 1015.5240 [M+H]⁺

### Example 167: N-(3-cyano-4-methoxyphenyl)-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

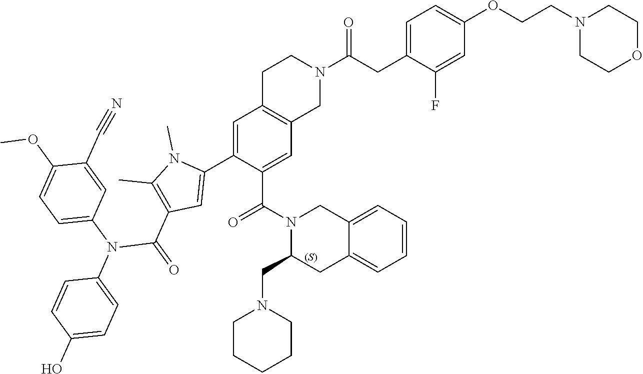

### Step A: 2-methoxy-5-({4-[(triisopropylsilyl)oxy]phenyl}amino)benzonitrile

Using General procedure 1a and 5-bromo-2-methoxybenzonitrile (300 mg, 1.41 mmol, 1 eq.) as the appropriate aryl bromide and Preparation IIIb (413 mg, 1.56 mmol, 1.1 eq.) as the appropriate aniline afforded the title product (341 mg, 0.86 mmol, 61%).

LRMS calcd for C₂₃H₃₂N₂O₂Si: 396.2, found 397.4 [M+H]⁺

¹H NMR (400 MHz, DMSO-d6) δ ppm: 7.87 (s, 1H), 7.22 (dd, J=9.0, 2.9 Hz, 1H), 7.15-7.09 (m, 2H), 6.96-6.90 (m, 2H), 6.82-6.76 (m, 2H), 3.83 (s, 3H), 1.29-1.14 (m, 3H), 1.06 (d, J=7.3 Hz, 18H).

### Step B: tert-butyl 6-{4-[(3-cyano-4-methoxyphenyl)({4-[(triisopropylsilyl)oxy]phenyl})carbamoyl]-1,5-dimethylpyrrol-2-yl}-7-{[(3S)-3-(piperidin-1-ylmethyl)-3,4-dihydro-1H-isoquinolin-2-yl]carbonyl}-3,4-dihydro-1H-isoquinoline-2-carboxylate

Using General procedure 2a and Preparation IIb (300 mg, 0.48 mmol, 1 eq.) as the appropriate acid and the product from Step A (285 mg, 0.72 mmol, 1.5 eq.) as the appropriate aniline afforded the title product (285.5 mg, 0.28 mmol, 59%).

LRMS calcd for C₆₀H₇₆N₆O₆Si: 1004.6, found 1005.6 [M+H]⁺

### Step C: N-(3-cyano-4-methoxyphenyl)-N-(4-hydroxyphenyl)-1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxamide

Using General procedure 3b and the product from Step B (285 mg, 0.28 mmol, 1 eq.) as the appropriate silyl-derivative afforded the title product (108.7 mg, 0.15 mmol, 51%).

LRMS calcd for C₄₆H₄₈N₆O₄: 748.4, found 749.6 [M+H]⁺

### Step D: N-(3-cyano-4-methoxyphenyl)-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and the product from Step C (108 mg, 0.14 mmol, 1 eq.) as the appropriate amine and Preparation Vb (49 mg, 0.17 mmol, 1.2 eq.) as the appropriate carboxylic acid afforded the title product (17.7 mg, 0.02 mmol, 12%).

HRMS calcd for C₆₀H₆₄FN₇O₇: 1013.4851, found 1014.4924 [M+H]⁺

### Example 168: N-(3-cyano-4-isopropoxyphenyl)-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

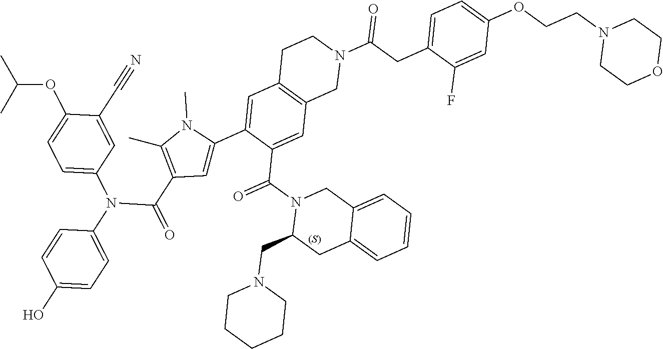

### Step A: 2-Isopropoxy-5-({4-[(triisopropylsilyl)oxy]phenyl}amino)benzonitrile

Using General procedure 1a and 5-bromo-2-isopropoxybenzonitrile (200 mg, 0.83 mmol, 1 eq.) as the appropriate aryl bromide and Preparation IIIb (243 mg, 0.92 mmol, 1.1 eq.) as the appropriate aniline afforded the title product (226.5 mg, 0.53 mmol, 64%).

LRMS calcd for C₂₅H₃₆N₂O₂Si: 424, found 425 [M+H]⁺

¹H NMR (400 MHz, DMSO-d6) δ ppm: 7.88 (s, 1H), 7.23-7.07 (m, 3H), 6.98-6.90 (m, 2H), 6.82-6.75 (m, 2H), 4.64-4.54 (m, 1H), 1.30-1.16 (m, 9H), 1.06 (d, J=7.3 Hz, 18H).

### Step B: tert-butyl 6-{4-[(3-cyano-4-isopropoxyphenyl)([4-[(triisopropylsilyl)oxy]phenyl})carbamoyl]-1,5-dimethylpyrrol-2-yl}-7-f[(3S)-3-(piperidin-1-ylmethyl)-3,4-dihydro-1H-isoquinolin-2-yl]carbonyl}-3,4-dihydro-1H-isoquinoline-2-carboxylate

Using General procedure 2a and Preparation IIb (140 mg, 0.22 mmol, 1 eq.) as the appropriate acid and the product from Step A (142 mg, 0.34 mmol, 1.5 eq.) as the appropriate aniline afforded the title product (103 mg, 0.1 mmol, 45%).

LRMS calcd for C₆₂H₈₀N₆O₆Si: 1032.6, found 1033.8 [M+H]⁺

### Step C: N-(3-cyano-4-isopropoxyphenyl)-N-(4-hydroxyphenyl)-1,2-dimethyl-5-(7-f[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxamide

Using General procedure 3b and the product from Step B (103 mg, 0.1 mmol, 1 eq.) as the appropriate silyl-derivative afforded the title product which was used in the subsequent step without further purification (73 mg, 0.09 mmol, 94%).

LRMS calcd for C₄₈H₅₂N₆O₄: 776.4, found 777.6 [M+H]⁺

### Step D: N-(3-cyano-4-isopropoxyphenyl)-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and the product from Step C (73 mg, 0.09 mmol, 1 eq.) as the appropriate amine and Preparation Vb (40 mg, 0.14 mmol, 1.5 eq.) as the appropriate carboxylic acid afforded the title product (6.2 mg, 0.01 mmol, 6%).

HRMS calcd for C₆₂H₆₈FN₇O₇: 1041.5164, found 1042.5239 [M+H]⁺

### Example 169: N-(5-cyano-1,2-dimethyl-pyrrol-3-yl)-5-[7-[(3S)-3-(ethoxymethyl)-3,4-dihydro-1H-isoquinoline-2-carbonyl]-2-[2-[2-fluoro-4-(2-morpholinoethoxy)phenyl]acetyl]-3,4-dihydro-1H-isoquinolin-6-yl]-N-(4-hydroxyphenyl)-1,2-dimethyl-pyrrole-3-carboxamide

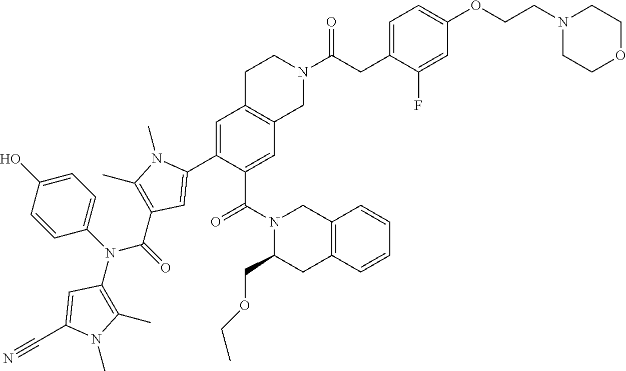 [SMILES: CCOC[C@@H]1Cc2ccccc2CN1C(=O)c1cc2c(cc1-c1cc(C(=O)N(c3ccc(O)cc3)c3cc(C#N)n(C)c3C)c(C)n1C)CCN(C(=O)Cc1ccc(OCCN3CCOCC3)cc1F)C2]

### Step A: [(3S)-2-(p-tolylsulfonyl)-3,4-dihydro-1H-isoquinolin-3-yl]methanol

To a solution of [(3S)-1,2,3,4-tetrahydroisoquinolin-3-yl]methanol (1.06 g, 6.49 mmol, 1 eq.) and DIPEA (1.50 mL, 8.61 mmol, 1.33 eq.) in DCM (20 mL) was added 4-methylbenzenesulfonyl chloride (1.325 g, 6.95 mmol, 1.07 eq.) portionwise over 5 min. After stirring at rt for 1 h, the mixture was diluted with DCM (50 mL) then the organic solution was washed with sat. aq. Na₂CO₃ solution (2×10 mL). The organic phase was dried over MgSO₄ and concentrated to furnish the targeted sulphonamide product which was further purified by automated flash chromatography using DCM and DCM/MeOH=9/1 as eluents to give the title compound (1.68 g, 82%).

¹H NMR (500 MHz, DMSO-d6) δ ppm 7.65 (m, 2H), 7.3 (m, 2H), 7.15-7.01 (m, 4H), 4.89 (dd, 1H), 4.5/4.25 (d+d, 2H), 4.03 (m, 1H), 3.36/3.17 (m+m, 2H), 2.78/2.61 (dd+dd, 2H), 2.32 (s, 3H); ¹³C NMR (125 MHz, DMSO-d6) δ ppm 130.1, 127.4, 61.7, 53.7, 44.3, 28.8, 21.4.

HRMS-ESI (m/z) [M+H]⁺ calcd for C₁₇H₂₀NO₃S: 318.1158, found 318.1158.

### Step B: (3S)-3-(ethoxymethyl)-2-(p-tolylsulfonyl)-3,4-dihydro-1H-isoquinoline

To the product from Step A (300 mg, 0.94 mmol) in DMF (3.8 mL) was added NaH (60 m/m %) (57 mg, 1.5 eq.) at 0° C. After 30 min, iodoethane (0.099 mL, 1.3 eq.) was added to the mixture, which was warmed to rt and stirred until complete conversion was observed. After the addition of water (50 mL), the mixture was extracted with EtOAc and the organic phase was washed with brine, dried, and concentrated to give the title compound (231 mg, 71%).

¹H NMR (500 MHz, DMSO-d6) δ ppm 7.67 (d, 2H), 7.31 (d, 2H), 7.15-7.11 (m, 3H), 7.06 (m, 1H), 4.50/4.25 (d+d, 2H), 4.23 (m, 1H), 3.36/3.20 (dd+dd, 2H), 3.16/3.13 (dd+dd, 2H), 2.72/2.69 (dd+dd, 2H), 2.33 (s, 3H), 0.88 (m, 1H), 0.40/0.09 (dd+dd, 4H); ¹³C NMR (125 MHz, DMSO-d6) δ ppm 130.1, 129.1, 127.5, 75.0, 70.1, 51.5, 44.2, 29.6, 21.4, 10.8, 3.3/3.2.

LC-MS (m/z) [M+H]⁺ calcd for C₂₁H₂₆NO₃S: 372.15, found 372.

### Step C: (3S)-3-(ethoxymethyl)-1,2,3,4-tetrahydroisoquinoline

To a solution of the product of Step A (300 mg, 0.84 mmol) in MeOH (13 mL) was added Mg powder (184 mg, 9.0 eq.) and the reaction mixture was stirred until the deprotection was complete. After filtration through a pad of Celite and concentration, the product was purified by flash chromatography using DCM and MeOH as eluents to give the title compound (125 mg, 58%).

¹H NMR (500 MHz, DMSO-d6) δ ppm 7.11-7.04 (m, 4H), 3.83/3.77 (d+d, 2H), 3.49/3.24 (dd+dd, 2H), 3.36 (q, 2H), 3.22 (m, 1H), 2.89/2.62 (dd+dd, 2H), 1.06 (t, 3H); ¹³C NMR (125 MHz, DMSO-d6) δ ppm 70.2, 66.1, 54.7, 49.4, 30.3, 15.6.

LC-MS (m/z) [M+H]⁺ calcd for C₁₂H₁₈NO: 192.13, found 192.

### Step D: N-(5-cyano-1,2-dimethyl-pyrrol-3-yl)-5-[7-[(3S)-3-(ethoxymethyl)-3,4-dihydro-1H-isoquinoline-2-carbonyl]-2-[2-[2-fluoro-4-(2-morpholinoethoxy)phenyl]acetyl]-3,4-dihydro-1H-isoquinolin-6-yl]-N-(4-hydroxyphenyl)-1,2-dimethyl-pyrrole-3-carboxamide

Using General procedure 4c, starting from Preparation IX (100 mg, 0.11 mmol) as the appropriate carboxylic acid and the product of Step C (34 mg, 1.2 eq.) as the appropriate amine, the title compound was obtained (49 mg, 46%).

HRMS-ESI (m/z) [M+H]⁺ calcd for C₅₆H₆₁FN₇O₇: 962.4611, found 962.4606.

### Example 170: N-(5-cyano-1,2-dimethyl-pyrrol-3-yl)-5-[7-[(3S)-3-(cyclopropylmethoxymethyl)-3,4-dihydro-1H-isoquinoline-2-carbonyl]-2-[2-[2-fluoro-4-(2-morpholinoethoxy)phenyl]acetyl]-3,4-dihydro-1H-isoquinolin-6-yl]-N-(4-hydroxyphenyl)-1,2-dimethyl-pyrrole-3-carboxamide

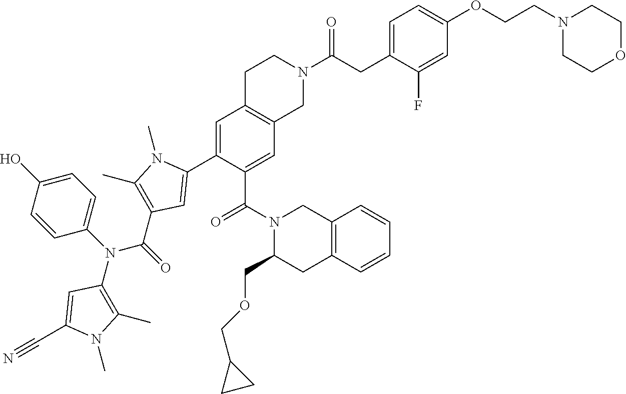 [SMILES: Cc1c(N(C(=O)c2cc(-c3cc4c(cc3C(=O)N3Cc5ccccc5C[C@H]3COCC3CC3)CN(C(=O)Cc3ccc(OCCN5CCOCC5)cc3F)CC4)n(C)c2C)c2ccc(O)cc2)cc(C#N)n1C]

### Step A: (3S)-3-(cyclopropylmethoxymethyl)-2-(p-tolylsulfonyl)-3,4-dihydro-1H-isoquinoline

To the product of Step A of Example 169 (500 mg, 1.57 mmol) in DMF (6.3 mL) was added NaH (60 m/m %) (95 mg, 1.5 eq.) at 0° C. After 30 min, bromomethylcyclopropane (276 mg, 1.3 eq.) was added to the mixture, which was warmed to rt and stirred until complete conversion was observed. After the addition of water (50 mL), the mixture was extracted with EtOAc and the organic phase was washed with brine, dried, and concentrated to give the title compound (505 mg, 86%).

¹H NMR (500 MHz, DMSO-d6) δ ppm 7.67 (m, 2H), 7.31 (m, 2H), 7.17-7.02 (m, 4H), 4.51/4.24 (d+d, 2H), 4.21 (m, 1H), 3.34 (m, 2H), 3.32/3.19 (dd+dd, 2H), 2.70 (d, 2H), 2.33 (s, 3H), 1.01 (t, 3H); ¹³C NMR (125 MHz, DMSO-d6) δ ppm 130.1, 127.5, 70.2, 66.1, 51.5, 44.1, 29.6, 21.4, 15.4.

LC-MS (m/z) [M+H]⁺ calcd for C₁₉H₂₄NO₃S: 346.14, found 346.0.

### Step B: (3S)-3-(cyclopropylmethoxymethyl)-1,2,3,4-tetrahydroisoquinoline

To a solution of the product of Step A (505 mg, 1.32 mmol) in MeOH (20 mL) was added Mg powder (288 mg, 9.0 eq.) and the reaction mixture was stirred until the deprotection was complete. After filtration through a pad of Celite and concentration the product was purified by flash chromatography using DCM and MeOH as eluents to give the title compound (270 mg, 66%).

¹H NMR (500 MHz, DMSO-d6) δ ppm 7.11-7.03 (m, 4H), 3.83/3.77 (d+d, 2H), 3.51/3.27 (dd+dd, 2H), 3.21 (dd+dd, 1H), 3.19/3.14 (dd+dd, 2H), 2.89/2.62 (m, 2H), 0.93 (m, 4H), 0.40/0.11 (dd+dd, 4H); ¹³C NMR (125 MHz, DMSO-d6) δ ppm 75.0, 70.2, 54.9, 49.4, 30.3, 11.0, 3.3.

LC-MS (m/z) [M+H]⁺ calcd for C₁₄H₂₀NO: 218.15, found 218.

### Step C. N-(5-cyano-1,2-dimethyl-pyrrol-3-yl)-5-[7-[(3S)-3-(cyclopropylmethoxymethyl)-3,4-dihydro-1H-isoquinoline-2-carbonyl]-2-[2-[2-fluoro-4-(2-morpholinoethoxy)phenyl]acetyl]-3,4-dihydro-1H-isoquinolin-6-yl]-N-(4-hydroxyphenyl)-1,2-dimethyl-pyrrole-3-carboxamide

Using General procedure 4c, starting from Preparation IX (100 mg, 0.11 mmol) as the appropriate carboxylic acid and the product of Step B (38 mg, 1.2 eq.) as the appropriate amine, the title compound was obtained (52 mg, 47%).

HRMS-ESI (m/z) [M+H]⁺ calcd for C₅₈H₆₃FN₇O₇: 988.4767, found 988.4771.

### Example 171: N-(5-cyano-1,2-dimethyl-pyrrol-3-yl)-5-[2-[2-[2,6-difluoro-4-(2-morpholinoethoxy)phenyl]acetyl]-7-[(3S)-3-(pyrrolidin-1-ylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

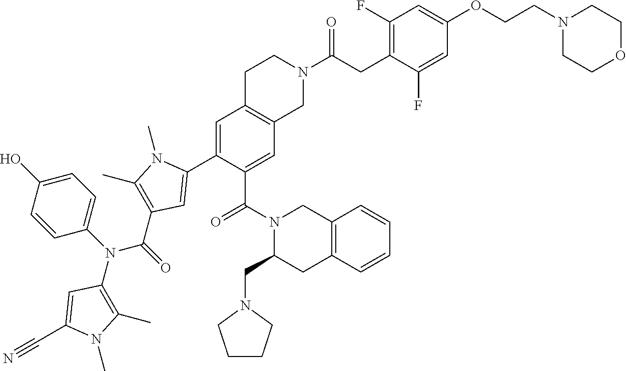 [SMILES: Cc1c(N(C(=O)c2cc(-c3cc4c(cc3C(=O)N3Cc5ccccc5C[C@H]3CN3CCCC3)CN(C(=O)Cc3c(F)cc(OCCN5CCOCC5)cc3F)CC4)n(C)c2C)c2ccc(O)cc2)cc(C#N)n1C]

### Step A: 1,2-dimethyl-5-[7-[(3S)-3-(pyrrolidin-1-ylmethyl)-3,4-dihydro-1H-isoquinoline-2-carbonyl]-1,2,3,4-tetrahydroisoquinolin-6-yl]pyrrole-3-carboxylic acid

Using the procedure described in Step A of Preparation VIIa, starting from Preparation I (1.5 g, 3.1 mmol) as the appropriate acid and the product of Step B of Preparation IIc (1.01 g, 1.1 eq.) as the appropriate amine, the title product was obtained (1.36 g, 84%).

¹H NMR (400 MHz, DMSO-d6) δ ppm 7.48-6.86 (m, 6H), 6.38-6.13 (s, 1H), 5.20-2.69 (m, 17H), 3.46-3.12 (s, 3H), 2.42-1.93 (s, 3H), 1.85-1.57 (m, 4H).

HRMS-ESI (m/z) [M+H]⁺ calcd for C₃₁H₃₇N₄O₃: 513.2860, found 513.2862.

### Step B: 5-[2-(9H-fluoren-9-ylmethoxycarbonyl)-7-[(3S)-3-(pyrrolidin-1-ylmethyl)-3,4-dihydro-1H-isoquinoline-2-carbonyl]-3,4-dihydro-1H-isoquinolin-6-yl-1,2-dimethyl-pyrrole-3-carboxylic acid hydrochloride

Using the procedure described in Step B of Preparation VIIa, starting from the product from Step A (1.36 g, 2.65 mmol), the title product was obtained as a hydrochloride salt (0.73 g, 36%).

¹H NMR (400 MHz, DMSO-d6) δ ppm 11.52 (brs, 1H), 10.65-10.16 (brm, 1H), 8.11-6.72 (m, 14H), 6.43-6.01 (brs, 1H), 5.45-1.76 (m, 24H), 3.48/3.09 (s, 3H), 1.91 (s, 3H).

HRMS-EST (m/z) [M+H]⁺ calcd for C₄₆H₄₇N₄O₅: 735.3541, found 735.3545.

### Step C. 9H-fluoren-9-ylmethyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-7-[(3S)-3-(pyrrolidin-1-ylmethyl)-3,4-dihydro-1H-isoquinoline-2-carbonyl]-3,4-dihydro-1H-isoquinoline-2-carboxylate

Using General procedure 2b, starting from the product of Step B (356 mg, 0.46 mmol) as the appropriate acid and the product of Step B of Preparation IVa (189 mg, 1.2 eq.) as the appropriate amine, afforded the title product (318 mg, 65%).

¹H NMR (500 MHz, DMSO-d6) δ ppm 8.03-6.43 (m, 19H), 5.41-1.37 (m, 24H), 5.40-4.80 (brs, 1H), 3.68-2.95 (s, 6H), 2.48-1.74 (s, 6H), 0.83 (s, 9H), 0.13-0.05 (s, 6H).

HRMS-ESI (m/z) [M+H]⁺ calcd for C₆₅H₇₂N₇O₅Si 1058.5359, found 1058.5353

### Step D: N-[4-[tert-butyl(dimethyl)silyl]oxyphenyl]-N-(5-cyano-1,2-dimethyl-pyrrol-3-yl)-1,2-dimethyl-5-[7-[(3S)-3-(pyrrolidin-1-ylmethyl)-3,4-dihydro-1H-isoquinoline-2-carbonyl]-1,2,3,4-tetrahydroisoquinolin-6-yl]pyrrole-3-carboxamide

Using the procedure described in Step D of Preparation VIIa, starting from the product obtained in Step C (318 mg, 0.30 mmol), the title product was obtained (167 mg, 66%).

¹H NMR (500 MHz, DMSO-d6) δ ppm 7.29-6.40 (m, 11H), 5.47-5.01 (s, 1H), 5.14-1.43 (m, 16H), 3.64-2.98 (s, 6H), 2.60-1.46 (m, 6H), 2.43-1.73 (s, 6H), 0.93-0.81 (s, 9H), 0.14-−0.07 (s, 6H).

HRMS-ESI (m/z) [M+H]⁺ calcd for C₅₀H₆₂N₇O₃Si 836.4678, found 836.4682.

### Step E: N-(5-cyano-1,2-dimethyl-pyrrol-3-yl)-5-[2-[2-[2,6-difluoro-4-(2-morpholinoethoxy)phenyl]acetyl]-7-[(3S)-3-(pyrrolidin-1-ylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

Using General Procedure 4c, starting from the product obtained in Example 97, Step D (46 mg, 1.6 eq.) as the appropriate acid and the product of Step E (80 mg. 0.096 mmol) as the appropriate amine, afforded the title product (54.1 mg, 56%).

HRMS-ESI (m/z) [M+H]⁺ calcd for C₅₈H₆₃F₂N₈O₆: 1005.4833, found 1005.4828.

### Example 172: N-[5-cyano-1-(difluoromethyl)-2-methyl-pyrrol-3-yl]-N-(4-hydroxyphenyl)-5-[7-[(3S)-3-(methoxymethyl)-3,4-dihydro-1H-isoquinoline-2-carbonyl]-2-[2-[4-(2-morpholinoethoxy)phenyl]acetyl]-3,4-dihydro-1H-isoquinolin-6-yl]-1,2-dimethyl-pyrrole-3-carboxamide

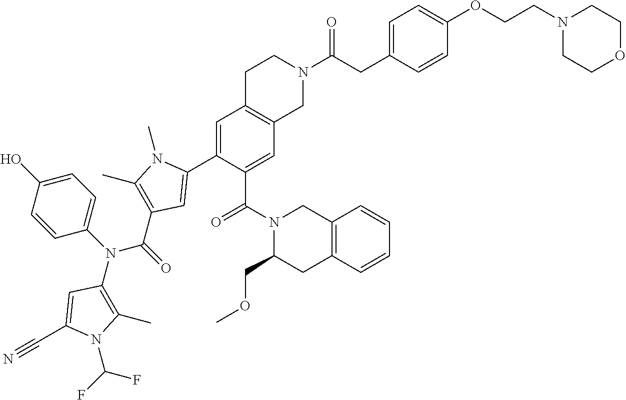 [SMILES: COC[C@@H]1Cc2ccccc2CN1C(=O)c1cc2c(cc1-c1cc(C(=O)N(c3ccc(O)cc3)c3cc(C#N)n(C(F)F)c3C)c(C)n1C)CCN(C(=O)Cc1ccc(OCCN3CCOCC3)cc1)C2]

### Step A: 02-benzyl 07-tert-butyl 6-[4-[(4-benzyloxyphenyl)-[5-cyano-1-(difluoromethyl)-2-methyl-pyrrol-3-yl]carbamoyl]-1,5-dimethyl-pyrrol-2-yl]-3,4-dihydro-1H-isoquinoline-2,7-dicarboxylate

Using General procedure 2b, starting from the product of Step B of Preparation IX (1.2 g, 2.38 mmol) as the appropriate acid and the product of Step A of Example 162 (1.0 g, 1.2 eq.) as the appropriate aniline, the title compound was obtained (1.1 g, 58%).

¹H NMR (500 MHz, DMSO-d6) δ ppm 7.81 (t, 1H), 7.62 (s, 1H), 7.42-7.28 (m, 10H), 7.21 (s, 1H), 7.11 (d, 2H), 6.95 (d, 2H), 6.87 (s, 1H), 5.19 (s, 1H), 5.13 (s, 2H), 5.06 (s, 2H), 4.68/4.62 (brs/brs, 2H), 3.63 (brm, 2H), 3.14 (s, 3H), 2.79 (t, 2H), 2.41 (s, 3H), 2.18 (s, 3H), 1.28 (s, 9H); ¹³C NMR (125 MHz, DMSO-d6) δ ppm 166.4, 166.3, 156.9, 155.1, 138.4, 137.4, 137.3, 137.3, 135.8, 134.3, 133.0, 132.3, 131.7, 131.2, 130.3, 128.8, 128.5, 128.1, 123.9, 115.5, 113.7, 112.5, 109.6, 108.2, 98.0, 81.2, 69.9, 66.9, 45.5/45.4, 41.4/41.0, 31.5, 28.5/28.3, 27.8, 12.0, 10.0.

HRMS-ESI (m/z) [M+H]⁺ calcd for C₄₉H₄₈F₂N₅O₆: 840.3567, found 840.3567.

### Step B: 2-benzyloxycarbonyl-6-[4-[(4-benzyloxyphenyl)-[5-cyano-1-(difluoromethyl)-2-methyl-pyrrol-3-yl]carbamoyl]-1,5-dimethyl-pyrrol-2-yl]-3,4-dihydro-1H-isoquinoline-7-carboxylic acid

Using General Procedure 3a, starting from the product of Step A (350 mg, 0.42 mmol) the title compound was obtained (250 mg, 76%).

¹H NMR (500 MHz, DMSO-d6) δ ppm 12.67 (brs, 1H), 7.76 (t, 1H), 7.74-6.72 (m, 17H), 5.13 (s, 1H), 5.13/5.08 (s, 4H), 4.76-4.56 (brs, 2H), 3.64 (brm, 2H), 3.13 (brs, 3H), 2.78 (brt, 2H), 2.41-2.12 (s, 6H).

HRMS-ESI (m/z) [M+H]⁺ calcd for C₄₅H₄₀F₂N₅O₆: 784.2941, found 784.2944.

### Step C. benzyl 6-[4-[(4-benzyloxyphenyl)-[5-cyano-1-(difluoromethyl)-2-methyl-pyrrol-3-yl]carbamoyl]-1,5-dimethyl-pyrrol-2-yl]-7-[(3S)-3-(methoxymethyl)-3,4-dihydro-1H-isoquinoline-2-carbonyl]-3,4-dihydro-1H-isoquinoline-2-carboxylate

Using General procedure 4c without the silyl deprotection step, starting from the product of

### Step B (250 mg, 0.32 mmol) as the appropriate carboxylic acid and the product of Step C of Example 52 (82 mg, 1.2 eq.) as the appropriate amine, the title compound was obtained (227 mg, 75%)

¹H NMR (500 MHz, DMSO-d6) δ ppm 7.81 (t, 1H), 7.47-6.77 (m, 21H), 5.70-5.12 (s, 1H), 5.38-1.38 (m, 11H), 5.21-4.91 (s, 4H), 3.39-2.75 (m, 2H), 3.38-3.05 (s, 3H), 3.29-2.88 (s, 3H), 2.46-1.92 (s, 6H).

HRMS-ESI (m/z) [M+H]⁺ calcd for C₅₆H₅₃F₂N₆O₆: 943.3989, found 943.3987.

### Step D: N-[5-cyano-1-(difluoromethyl)-2-methyl-pyrrol-3-yl]-N-(4-hydroxyphenyl)-5-[7-[(3S)-3-(methoxymethyl)-3,4-dihydro-1H-isoquinoline-2-carbonyl]-1,2,3,4-tetrahydroisoquinolin-6-yl]-1,2-dimethyl-pyrrole-3-carboxamide

To the product of Step C (220 mg, 0.23 mmol) in methanol (3.5 mL) was added 10% Pd on charcoal (12 mg, 0.5 eq.). The reaction mixture was stirred under hydrogen atmosphere until complete conversion was observed. The mixture was filtered through a pad of Celite, concentrated, and dried to give the title product (125 mg, 74%).

¹H NMR (500 MHz, DMSO-d6) δ ppm 9.61-9.29 (brs, 1H), 7.80 (t, 1H), 7.28-6.52 (m, 11H), 5.65-5.05 (s, 1H), 5.37-1.80 (m, 12H), 3.37-2.80 (s, 2H), 3.36-3.02 (s, 3H), 3.27-2.91 (s, 3H), 2.44-1.90 (s, 6H).

HRMS-ESI (m/z) [M+H]⁺ calcd for C₄₁H₄₁F₂N₆O₄: 719.3152, found 719.3148.

### Step E: N-[5-cyano-1-(difluoromethyl)-2-methyl-pyrrol-3-yl]-N-(4-hydroxyphenyl)-5-[7-[(3S)-3-(methoxymethyl)-3,4-dihydro-1H-isoquinoline-2-carbonyl-2-[2-[4-(2-morpholinoethoxy)phenyl]acetyl]-3,4-dihydro-1H-isoquinolin-6-yl-1,2-dimethyl-pyrrole-3-carboxamide

Using General procedure 4c, starting from the Preparation Va (48 mg, 1.1 eq.) as the appropriate carboxylic acid and the product of Step D (118 mg, 1.0 mmol) as the appropriate amine, the title compound was obtained (70 mg, 44%).

HRMS-ESI (m/z) [M+H]⁺ calcd for C₅₅H₅₈F₂N₇O₇: 966.4360, found 966.4364.

### Example 173: N-(5-cyano-1,2-dimethylpyrrol-3-yl)-5-[2-(2-{2-fluoro-4-[2-(morpholin-4-yl)ethoxy]phenyl}acetyl)-7-{[(3S)-3-{[(2S)-2-methylpyrrolidin-1-yl]methyl}-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

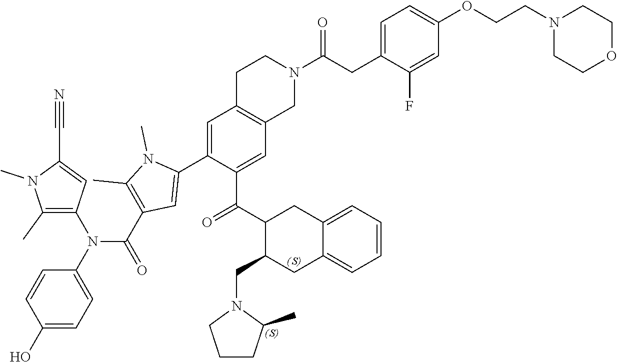

### Step A: tert-butyl (3S)-3-{[(2S)-2-methylpyrrolidin-1-yl]carbonyl}-3,4-dihydro-1H-isoquinoline-2-carboxylate

Using General procedure 5 and Boc-(3S)-1,2,3,4-tetrahydroisoquinoline-3-carboxylic acid (3 g, 10.82 mmol, 1 eq.) and (S)-2-methyl-pyrrolidine hydrochloride (1.38 g, 11.36 mmol, 1.05 eq.) as the appropriate amine afforded the title product (3.36 g, 9.75 mmol, 90%). LRMS calcd for C₂₀H₂₈N₂O₃: 344.2, found 345.4 [M+H]⁺

¹H NMR (400 MHz, DMSO-d6) δ ppm: 7.27-7.06 (m, 4H), 4.97-4.46 (m, 2H), 4.45-3.83 (m, 2H), 3.64-2.73 (m, 4H), 2.05-0.95 (m, 16H).

### Step B: (3S)-3-{[(2S)-2-methylpyrrolidin-1-yl]methyl}-1,2,3,4-tetrahydroisoquinoline dihydrochloride

Using General procedure 6 and the product from Step A (3.36 g, 9.75 mmol, 1 eq.) as the appropriate THIQ amide afforded the title product (2.58 g, 8.5 mmol, 87%).

LRMS calcd for C₁₅H₂₂N₂: 230.2, found 231.2 [M+H]⁺

¹H NMR (400 MHz, DMSO-d6) δ ppm: 10.87 (br s, 1H), 10.63 (br s, 1H), 10.37 (br s, 1H), 7.36-7.15 (m, 4H), 4.47-4.33 (m, 2H), 4.14-4.02 (m, 1H), 4.01-3.80 (m, 2H), 3.61-3.02 (m, 5H), 2.32-2.18 (m, 1H), 2.11-1.89 (m, 2H), 1.74-1.57 (m, 1H), 1.57-1.38 (m, 3H).

### Step C: N-(5-cyano-1,2-dimethylpyrrol-3-yl)-5-[2-(2-{2-fluoro-4-[2-(morpholin-4-yl)ethoxy]phenyl}acetyl)-7-{[(3S)-3-{[(2S)-2-methylpyrrolidin-1-yl]methyl}-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and the product from Step B (53 mg, 0.17 mmol, 1.05 eq.) as the appropriate amine and Preparation IX (150 mg, 0.17 mmol, 1 eq) as the appropriate carboxylic acid afforded an intermediate which was treated according to General procedure 3b to afford the title product (66.8 mg, 0.07 mmol, 40%).

HRMS calcd for C₅₉H₆₅FN₈O₆: 1000.5011, found 1001.5082 [M+H]⁺

### Example 174: N-(5-cyano-1,2-dimethyl-pyrrol-3-yl)-5-[2-[2-[4-[2-((2S or R)-1,4-dioxan-2-yl)ethoxy]-2-fluoro-phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereoisomer 1)

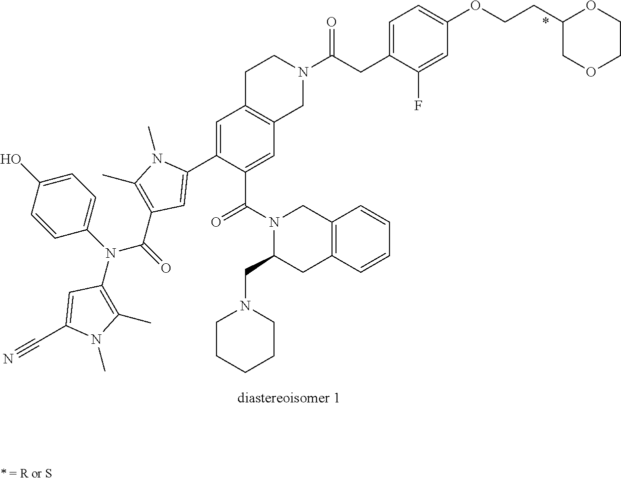 [SMILES: Cc1c(N(C(=O)c2cc(-c3cc4c(cc3C(=O)N3Cc5ccccc5C[C@H]3CN3CCCCC3)CN(C(=O)Cc3ccc(OCCC5COCCO5)cc3F)CC4)n(C)c2C)c2ccc(O)cc2)cc(C#N)n1C]

### Step A: methyl 2-[4-[2-((2S)-1,4-dioxan-2-yl)ethoxy]-2-fluoro-phenyl]acetate and methyl 2-[4-[2-((2R)-1,4-dioxan-2-yl)ethoxy]-2-fluoro-phenyl]acetate

Using the General procedure 7c, starting from methyl 2-(2-fluoro-4-hydroxy-phenyl)acetate (0.30 g, 1.62 mmol) as the appropriate phenol and 2-(1,4-dioxan-2-yl)ethanol (0.32 g, 1.5 eq.) as the appropriate alcohol, the desired product was obtained (470 mg, 97%).

¹H NMR (500 MHz, DMSO-d6) δ ppm 7.22 (t, 1H), 6.81 (dd, 1H), 6.74 (dm, 1H), 4.04/4.02 (m+m, 2H), 3.72/3.22 (dd+dd, 2H), 3.70/3.56 (m+m, 2H), 3.64 (m, 1H), 3.63 (brs, 2H), 3.63/3.45 (m+m, 2H), 3.61 (s, 3H), 1.77/1.73 (m+m, 2H); ¹³C NMR (125 MHz, DMSO-d6) δ ppm 171.5, 132.7, 111.0, 102.3, 72.4, 70.9, 66.6, 66.3, 64.6, 52.3, 33.6, 31.2.

HRMS-ESI (m/z) [M+H]⁺ calcd for C₁₅H₂₀FO₅: 299.1285, found 299.1287.

The enantiomers of the desired product was separated on a Regis (R,R) WHELK-O chiral column (250*4.6 mm, 5 m) using 10:90 EtOH-heptane as eluents to give the Enantiomer 1 (99.8% ee) and Enantiomer 2 (95.6% ee).

### Step B: 2-[4-[2-((2S or R)-1,4-dioxan-2-yl)ethoxy]-2-fluoro-phenyl]acetic acid (from Enantiomer 1)

Using the General procedure 8, starting from the Enantiomer 1 of Step A (190 mg, 0.64 mmol), the desired product was obtained (170 mg, 94%).

¹H NMR (400 MHz, DMSO-d6) δ ppm 12.37 (s, 1H), 7.21 (t, 1H), 6.79 (dd, 1H), 6.72 (dd, 1H), 4.03 (t, 2H), 3.77-3.41 (m, 4H), 3.73/3.22 (dd+dd, 2H), 3.65 (m, 1H), 3.52 (s, 2H), 1.84-1.65 (m, 2H); ¹³C NMR (100 MHz, DMSO-d6) δ ppm 172.5, 132.7, 110.9, 102.2, 72.4, 70.9, 66.6/66.3, 64.5, 34.0, 31.2.

HRMS-ESI (m/z) [M+NH₄]⁺ calcd for C₁₄H₁₈FO₅: 302.1398, found 302.1399.

### Step C: N-(5-cyano-1,2-dimethyl-pyrrol-3-yl)-5-[2-[2-[4-[2-((2S or R)-1,4-dioxan-2-yl)ethoxy]-2-fluoro-phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereoisomer 1)

Using General procedure 4c, starting from the product of Step B (32 mg, 1.2 eq.) as the appropriate carboxylic acid and Preparation VIIa (80 mg, 0.09 mmol) as the appropriate amine, the title compound was obtained (70 mg, 74%).

HRMS-ESI (m/z) [M+H]⁺ calcd for C₅₉H₆₅FN₇O₇: 1002.4924, found 1002.4928.

### Example 175: N-(5-cyano-1,2-dimethyl-pyrrol-3-yl)-5-[2-[2-[4-[2-((2R or S)-1,4-dioxan-2-yl)ethoxy]-2-fluoro-phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereoisomer 2)

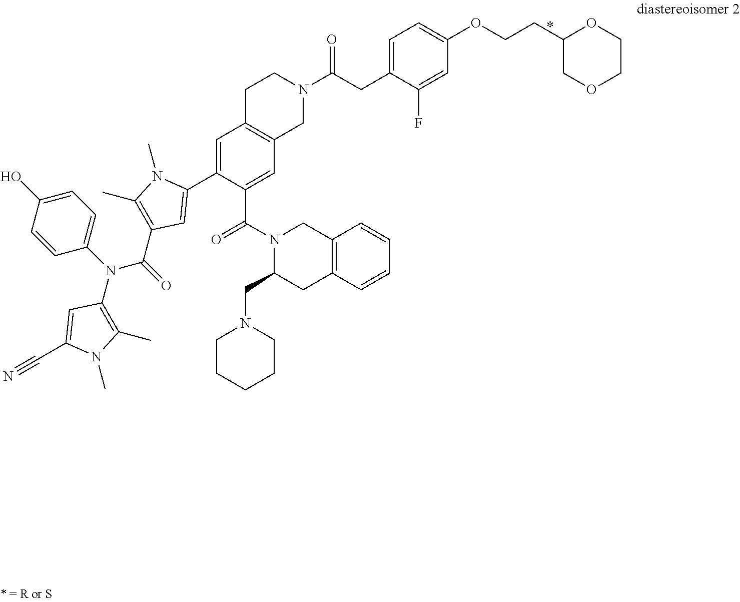 [SMILES: Cc1c(N(C(=O)c2cc(-c3cc4c(cc3C(=O)N3Cc5ccccc5C[C@H]3CN3CCCCC3)CN(C(=O)Cc3ccc(OCCC5COCCO5)cc3F)CC4)n(C)c2C)c2ccc(O)cc2)cc(C#N)n1C]

### Step A: 2-[4-[2-((2R or S)-1,4-dioxan-2-yl)ethoxy]-2-fluoro-phenyl]acetic acid (from Enantiomer 2)

Using the General procedure 8, starting from the Enantiomer 2 of Step A of Example 174 (210 mg, 0.68 mmol), the desired product was obtained (150 mg, 56%).

¹H NMR (400 MHz, DMSO-d6) δ ppm 12.37 (s, 1H), 7.21 (t, 1H), 6.79 (dd, 1H), 6.72 (dd, 1H), 4.03 (t, 2H), 3.77-3.41 (m, 4H), 3.73/3.22 (dd+dd, 2H), 3.65 (m, 1H), 3.52 (s, 2H), 1.84-1.65 (m, 2H); ¹³C NMR (100 MHz, DMSO-d6) δ ppm 172.5, 132.7, 110.9, 102.2, 72.4, 70.9, 66.6/66.3, 64.5, 34.0, 31.2.

HRMS-ESI (m/z) [M+NH₄]⁺ calcd for C₁₄H₁₈FO₅: 302.1398, found 302.1399.

### Step B: N-(5-cyano-1,2-dimethyl-pyrrol-3-yl)-5-[2-[2-[4-[2-((2R or S)-1,4-dioxan-2-yl)ethoxy]-2-fluoro-phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide (Diastereoisomer 2)

Using General procedure 4c, starting from the product of Step A (32 mg, 1.2 eq.) as the appropriate carboxylic acid and Preparation VIIa (80 mg, 0.09 mmol) as the appropriate amine, the title compound was obtained (63 mg, 67%).

HRMS-ESI (m/z) [M+H]⁺ calcd for C₅₉H₆₅FN₇O₇: 1002.4924, found 1002.4929.

### Example 176: 5-[7-[(3S)-3-(5-azaspiro[2.4]heptan-5-ylmethyl)-3,4-dihydro-1H-isoquinoline-2-carbonyl]-2-[2-[2-fluoro-4-(2-morpholinoethoxy)phenyl]acetyl]-3,4-dihydro-1H-isoquinolin-6-yl]-N-(5-cyano-1,2-dimethyl-pyrrol-3-yl)-N-(4-hydroxyphenyl)-1,2-dimethyl-pyrrole-3-carboxamide

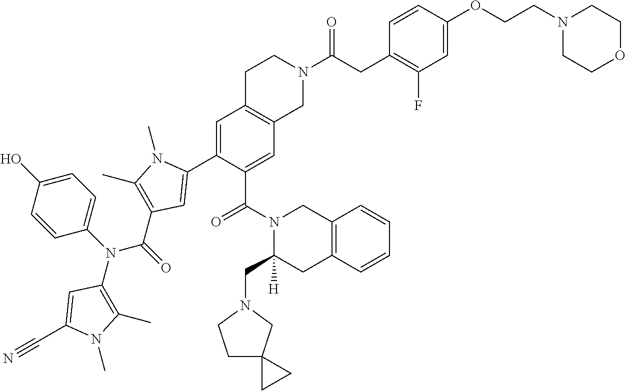 [SMILES: Cc1c(N(C(=O)c2cc(-c3cc4c(cc3C(=O)N3Cc5ccccc5C[C@H]3CN3CCC5(CC5)C3)CN(C(=O)Cc3ccc(OCCN5CCOCC5)cc3F)CC4)n(C)c2C)c2ccc(O)cc2)cc(C#N)n1C]

### Step A: 5-azaspiro[2.4]heptan-5-yl-[(3S)-1,2,3,4-tetrahydroisoquinolin-3-yl]methanone

Using General procedure 5a, starting from (3S)-2-benzyloxycarbonyl-3,4-dihydro-1H-isoquinoline-3-carboxylic acid (600 mg, 1.93 mmol) and 5-azaspiro[2.4]heptane hydrochloride (310 mg, 1.2 eq.) as reactants the title product was obtained after deprotection according to

General procedure 6b (360 mg, 91%).

¹H NMR (500 MHz, DMSO-d6) δ ppm 7.15-6.96 (m, 4H), 3.98-3.82 (m, 2H), 3.89-3.43 (m, 2H), 3.71/3.58 (m, 1H), 3.57/3.42/3.26/3.21 (d+d, 2H), 2.87-2.66 (m, 2H), 2.31-2.19 (m, 1H), 1.92-1.67 (m, 2H), 0.64-0.52 (m, 4H).

HRMS-ESI (m/z) [M+H]⁺ calcd for C₁₆H₂₁N₂O: 257.1648, found 256.1647.

### Step B: (3S)-3-(5-azaspiro[2.4]heptan-5-ylmethyl)-1,2,3,4-tetrahydroisoquinoline

Using General procedure 6a, starting from the product of Step A (350 mg, 1.37 mmol), the title product was obtained (300 mg, 91%).

¹H NMR (500 MHz, DMSO-d6) δ ppm 7.12-6.97 (m, 4H), 3.90 (s, 2H), 2.85 (m, 1H), 2.71/2.63 (m+m, 2H), 2.7/2.39 (dd+dd, 2H), 2.51/2.39 (dd+dd, 2H), 2.50/2.43 (d+d, 2H), 2.40 (brs, 1H), 1.75/1.72 (m+m, 2H), 0.59-0.45 (m, 4H); ¹³C NMR (125 MHz, DMSO-d6) δ ppm 63.6, 62.4, 55.7, 52.5, 48.5, 34.5, 34.3.

HRMS-ESI (m/z) [M+H]⁺ calcd for C₁₆H₂₃N₂: 243.1856, found 243.1857.

### Step C. 5-[7-[(3S)-3-(5-azaspiro[2.4]heptan-5-ylmethyl)-3,4-dihydro-1H-isoquinoline-2-carbonyl]-2-[2-[2-fluoro-4-(2-morpholinoethoxy)phenyl]acetyl]-3,4-dihydro-1H-isoquinolin-6-yl]-N-(5-cyano-1,2-dimethyl-pyrrol-3-yl)-N-(4-hydroxyphenyl)-1,2-dimethyl-pyrrole-3-carboxamide

Using General procedure 4c, starting from Preparation IX (80 mg, 0.089 mmol) as the appropriate carboxylic acid and the product from Step B (30 mg, 1.4 eq.) as the appropriate amine, afforded the desired product (53 mg, 59%).

HRMS-ESI (m/z) [M+H]⁺ calcd for C₆₀H₆₆FN₈O₆: 1013.5084, found 1013.5082.

### Example 177: N-(5-cyano-6-methyl-3-pyridyl)-5-[2-[2-[2-fluoro-4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

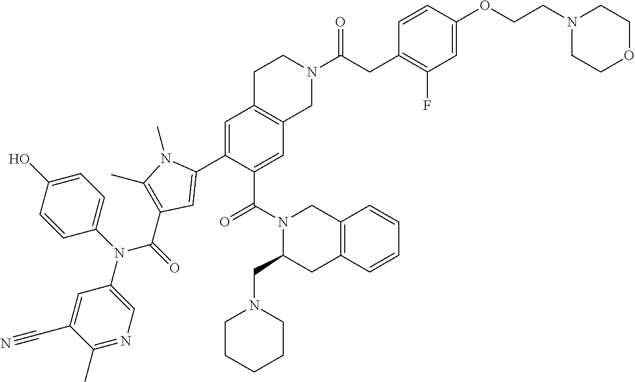 [SMILES: Cc1ncc(N(C(=O)c2cc(-c3cc4c(cc3C(=O)N3Cc5ccccc5C[C@H]3CN3CCCCC3)CN(C(=O)Cc3ccc(OCCN5CCOCC5)cc3F)CC4)n(C)c2C)c2ccc(O)cc2)cc1C#N]

### Step A: 2-methyl-5-({4-[(triisopropylsilyl)oxy]phenyl}amino)pyridine-3-carbonitrile

Using General procedure 1a and 5-bromo-2-methylnicotinonitrile (690 mg, 3.5 mmol, 1 eq.) as the appropriate aryl bromide and Preparation IIIb (976 mg, 3.68 mmol, 1.05 eq.) as the appropriate aniline afforded the title product (419 mg, 1.1 mmol, 31%).

LRMS calcd for C₂₂H₃₁N₃OSi: 381.2, found 382.2 [M+H]⁺

¹H NMR (400 MHz, DMSO-d6) δ ppm: 8.36 (s, 1H), 8.34 (d, J=2.9 Hz, 1H), 7.52 (d, J=2.9 Hz, 1H), 7.09-7.02 (m, 2H), 6.88-6.81 (m, 2H), 2.53 (s, 3H), 1.32-1.18 (m, 3H), 1.07 (d, J=7.3 Hz, 18H).

### Step B: tert-butyl 6-{4-[(5-cyano-6-methylpyridin-3-yl)({4-[(triisopropylsilyl)oxy]phenyl})carbamoyl]-1,5-dimethylpyrrol-2-yl}-7-f[(3S)-3-(piperidin-1-ylmethyl)-3,4-dihydro-1H-isoquinolin-2-yl]carbonyl}-3,4-dihydro-1H-isoquinoline-2-carboxylate

Using General procedure 2a and Preparation IIb (300 mg, 0.48 mmol, 1 eq.) as the appropriate acid and the product from Step A (274 mg, 0.72 mmol, 1.5 eq.) as the appropriate aniline afforded the title product (49 mg, 0.05 mmol, 10%).

LRMS calcd for C₅₉H₇₅N₇O₅Si: 989.6, found 990.6 [M+H]⁺

### Step C: N-(5-cyano-6-methylpyridin-3-yl)-N-(4-hydroxyphenyl)-1,2-dimethyl-5-(7-{[(3S)-3-(piperidin-1-ylmethyl)-3,4-dihydro-1H-isoquinolin-2-yl]carbonyl}-1,2,3,4-tetrahydroisoquinolin-6-yl)pyrrole-3-carboxamide dihydrochloride

Using General procedure 3b and the product from Step B (49 mg, 0.05 mmol, 1 eq.) as the appropriate silyl-derivative afforded the title product (39 mg, 0.05 mmol, 98%).

LRMS calcd for C₄₅H₄₇N₇O₃: 733.4, found 734.4 [M+H]⁺

### Step D: N-(5-cyano-6-methylpyridin-3-yl)-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide

Using General procedure 4a and the product from Step C (36 mg, 0.05 mmol, 1 eq.) as the appropriate amine and Preparation Vb (21 mg, 0.07 mmol, 1.5 eq.) as the appropriate carboxylic acid afforded the title product (7.1 mg, 0.01 mmol, 14%).

HRMS calcd for C₅₉H₆₃FN₈O₆: 998.4855, found 999.4924 [M+H]⁺

### Example 178: N-(5-cyano-1,2-dimethyl-pyrrol-3-yl)-5-[2-[2,2-difluoro-2-[4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

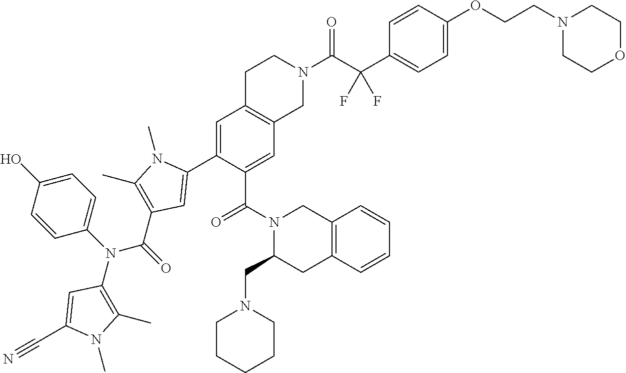 [SMILES: Cc1c(N(C(=O)c2cc(-c3cc4c(cc3C(=O)N3Cc5ccccc5C[C@H]3CN3CCCCC3)CN(C(=O)C(F)(F)c3ccc(OCCN5CCOCC5)cc3)CC4)n(C)c2C)c2ccc(O)cc2)cc(C#N)n1C]

### Step A: ethyl 2-[4-(2-bromoethoxy)phenyl]-2-oxo-acetate

To a suspension of AlCl₃ (2.00 g, 15 mmol) in CHCl₃ (35 mL) was added ethyl 2-chloro-2-oxo-acetate (1.00 g, 7.6 mmol) and a solution 2-bromoethoxybenzene (1.50 g, 7.5 mmol) in CHCl₃ (10 mL) under vigorous stirring at room temperature for 3 h. The reaction mixture was poured onto ice (35 mL) and then 20 mL of concentrated hydrochloric acid was added. The organic layer was dried over sodium sulphate then concentrated and the residue was purified by automated flash chromatography using heptane and EtOAc as eluents to give the title compound (0.959 g, 37%).

¹H NMR (500 MHz, DMSO-d6) δ ppm 7.94 (d, 2H), 7.18 (d, 2H), 4.48 (t, 2H), 4.41 (q, 2H), 3.85 (t, 2H), 1.32 (t, 3H); ¹³C NMR (125 MHz, DMSO-d6) δ ppm 185.7, 164.7, 164.0, 132.8, 125.3, 115.8, 68.8, 62.7, 31.5, 14.4.

HRMS-ESI (m/z) [M+H]⁺ calcd for C₁₂H₁₄BrO₄: 301.0070, found 301.0066.

### Step B: ethyl 2-[4-(2-bromoethoxy)phenyl]-2,2-difluoro-acetate

To the solution of the product from Step A (951 mg, 3.16 mmol) in DCM (29 mL) was added DAST (0.835 mL, 6.32 mmol) dropwise at 0° C. The reaction mixture was allowed to warm to room temperature and was stirred overnight. The reaction mixture was poured into ice water then partitioned between DCM and water, then the combined organic phases were dried (Na₂SO₄) and concentrated in vacuo. The crude material was purified by automated flash chromatography using heptane and EtOAc as eluents to give the title compound (0.726 g, 65%).

¹H NMR (500 MHz, DMSO-d6) δ ppm 7.52 (m, 2H), 7.11 (m, 2H), 4.39 (m, 2H), 4.30 (q, 2H), 3.82 (m, 2H), 1.22 (t, 3H); ¹³C NMR (125 MHz, DMSO-d6) δ ppm 163.9, 160.6, 127.4, 124.8, 115.5, 114.0, 68.4, 63.8, 31.7, 14.1.

HRMS-EI (m/z) [M+H]⁺ calcd for C₁₂H₁₃BrF₂O₃: 322.0016, found 322.0005.

### Step C: 2,2-difluoro-2-[4-(2-morpholinoethoxy)phenyl]acetic acid

Using General procedure 7d and then General procedure 8, starting from the product of Step B (323 mg, 1.00 mmol) as the appropriate alkyl halide and morpholine (0.262 mL, 3 mmol) as the appropriate amine, the title product was obtained (258 mg, 85%).

¹H NMR (500 MHz, DMSO-d6) δ ppm 13.31 (brs, 1H), 7.44 (m, 2H), 6.88 (m, 2H), 4.23 (t, 2H), 3.79 (m, 4H), 3.28 (t, 2H), 3.07 (brm, 4H); ¹³C NMR (125 MHz, DMSO-d6) δ ppm 127.2, 115.3, 114.6, 64.5, 63.6, 55.7, 52.6.

HRMS-ESI (m/z) [M+H]⁺ calcd for C₁₄H₁₈F₂NO₄: 302.1198, found 302.1200.

### Step D: N-(5-cyano-1,2-dimethyl-pyrrol-3-yl)-5-[2-[2,2-difluoro-2-[4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

Using General procedure 4c, starting from the product of Step C (42.5 mg, 1.5 eq.) as the appropriate carboxylic acid and Preparation VIIa (80 mg, 0.09 mmol) as the appropriate amine, the title compound was obtained (23 mg, 24%).

HRMS-ESI (m/z) [M+H]⁺ calcd for C₅₉H₆₅F₂N₈O₆: 1019.4990, found 1019.4994.

### Example 179: N-(3-cyano-4-methoxy-phenyl)-5-[2-[2-[2-fluoro-4-(2-morpholinoethoxy)phenyl]acetyl]-7-[(3S)-3-[[isopropyl(methyl)amino]methyl]-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

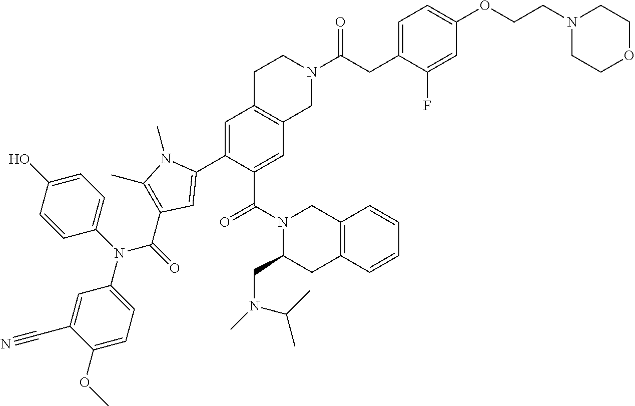 [SMILES: COc1ccc(N(C(=O)c2cc(-c3cc4c(cc3C(=O)N3Cc5ccccc5C[C@H]3CN(C)C(C)C)CN(C(=O)Cc3ccc(OCCN5CCOCC5)cc3F)CC4)n(C)c2C)c2ccc(O)cc2)cc1C#N]

### Step A: 02-benzyl 07-tert-butyl 6-[4-[[4-[tert-butyl(dimethyl)silyl]oxyphenyl]-(3-cyano-4-methoxy-phenyl)carbamoyl]-1,5-dimethyl-pyrrol-2-yl]-3,4-dihydro-1H-isoquinoline-2,7-dicarboxylate

Using General procedure 2b, starting from the product of Step B of Preparation IX (700 mg, 1.39 mmol) as the appropriate acid and the product of Step A of Example 81 (590 mg, 1.2 eq.) as the appropriate aniline, the title compound was obtained (860 mg, 74%).

¹H NMR (500 MHz, DMSO-d6) δ ppm 7.60 (s, 1H), 7.55 (d, 1H), 7.46 (dd, 1H), 7.42-7.29 (m, 5H), 7.21 (d, 1H), 7.09 (dm, 2H), 6.86 (s, 1H), 6.77 (dm, 2H), 5.12 (s, 2H), 5.03 (s, 1H), 4.66/4.60 (br/br., 2H), 3.89 (s, 3H), 3.63 (br., 2H), 3.14 (s, 3H), 2.78 (t, 2H), 2.43 (s, 3H), 1.28 (s, 9H), 0.83 (s, 9H), 0.07 (s, 6H); ¹³C NMR (125 MHz, DMSO-d6) δ ppm 166.5, 166.3, 159.0, 155.1, 154.0, 138.4, 138.2, 137.3, 134.7, 132.8, 132.5, 129.8, 128.1, 120.9, 116.4, 113.3, 108.8, 100.7, 66.8, 57.0, 45.5, 41.4/41.1, 31.5, 28.6/28.4, 27.8, 25.9, 12.2, −4.2.

HRMS-ESI (m/z) [M+H]⁺ calcd for C₄₉H₅₇N₄O₇Si: 841.3991, found 841.3995.

### Step B: tert-butyl 6-[4-[[4-[tert-butyl(dimethyl)silyl]oxyphenyl]-(3-cyano-4-methoxy-phenyl)carbamoyl]-1,5-dimethyl-pyrrol-2-yl]-1,2,3,4-tetrahydroisoquinoline-7-carboxylate

Using General procedure 6b, starting from the product of Step A (850 mg, 1.01 mmol) afforded the title product (693 mg, 97%).

¹H NMR (500 MHz, DMSO-d6) δ ppm 7.54 (d, 1H), 7.46 (dd, 1H), 7.42 (s, 1H), 7.21 (d, 1H), 7.09 (dm, 2H), 6.78 (dm, 2H), 6.72 (s, 1H), 5.02 (s, 1H), 3.89 (s, 3H), 3.85 (s, 2H), 3.12 (s, 3H), 2.91 (t, 2H), 2.64 (t, 2H), 2.43 (s, 3H), 1.27 (s, 9H), 0.86 (s, 9H), 0.09 (s, 6H); ¹³C NMR (125 MHz, DMSO-d6) δ ppm 166.5, 166.4, 159.0, 154.0, 138.3, 138.2, 134.7, 133.2, 132.5, 129.8, 127.9, 120.9, 116.4, 113.3, 108.6, 100.7, 57.0, 47.8, 43.3, 31.4, 29.2, 27.9, 25.9, 12.2, −4.2.

HRMS-ESI (m/z) [M+H]⁺ calcd for C₄₁H₅₁N₄O₅Si: 707.3623, found 707.3623.

### Step C. tert-butyl 6-[4-[[4-[tert-butyl(dimethyl)silyl]oxyphenyl]-(3-cyano-4-methoxy-phenyl)carbamoyl]-1,5-dimethyl-pyrrol-2-yl]-2-[2-[2-fluoro-4-(2-morpholinoethoxy)phenyl]acetyl]-3,4-dihydro-1H-isoquinoline-7-carboxylate

Using the procedure described in Preparation IX, Step E and replacing the product from Preparation Vb, Step C with the product of Step B (690 mg, 0.976 mmol) as the appropriate amine afforded the title compound (926 mg, 98%).

¹H NMR (500 MHz, DMSO-d6) δ ppm 7.60/7.59 (s/s, 1H), 7.55 (d, 1H), 7.46 (dd, 1H), 7.21 (d, 1H), 7.13 (dd, 1H), 7.09 (d, 2H), 6.88/6.87 (s/s, 1H), 6.79/6.78 (dd/dd, 1H), 6.78 (d, 2H), 6.72/6.70 (dd/dd, 1H), 5.05/5.04 (s/s, 1H), 4.81/4.65 (s/s, 2H), 4.07/4.06 (t/t, 2H), 3.90 (s, 3H), 3.75/3.67 (t/t, 2H), 3.73 (s, 2H), 3.57 (t, 4H), 3.15/3.14 (s/s, 3H), 2.82/2.75 (t/t, 2H), 2.67 (t, 2H), 2.45 (t, 4H), 2.44/2.43 (s/s, 3H), 1.29/1.28 (s/s, 9H), 0.85/0.84 (s/s, 9H), 0.09/0.08 (s/s, 6H); ¹³C NMR (125 MHz, DMSO-d6) δ ppm 134.7, 132.7/132.6, 132.5, 132.5, 129.8, 128.2/128.1, 120.9, 113.3, 110.9, 108.8, 102.2, 66.6, 66.2, 57.4, 57, 54.1, 46.6/43.9, 42.8/39.4, 33.3/33.0, 31.5, 29.0/28.3, 27.8, 26.0, 12.2, −4.2.

HRMS-ESI (m/z) [M+H]⁺ calcd for C₅₅H₆₇FN₅O₈Si: 972.4737, found 972.4746.

### Step D: 6-[4-[[4-[tert-butyl(dimethyl)silyl]oxyphenyl]-(3-cyano-4-methoxy-phenyl)carbamoyl]-1,5-dimethyl-pyrrol-2-yl]-2-[2-[2-fluoro-4-(2-morpholinoethoxy)phenyl]acetyl]-3,4-dihydro-1H-isoquinoline-7-carboxylic acid

Using General Procedure 3a, starting from the product of Step C (920 mg, 0.823 mmol), followed with a purification step via automated flash chromatography using DCM and methanol as eluents afforded the title product (645 mg, 86%).

¹H NMR (500 MHz, DMSO-d6) δ ppm 12.63 (brs, 1H), 7.71-6.67 (m, 12H), 5.06 (s, 1H), 4.81/4.66 (s, 2H), 4.60-2.20 (brm, 12H), 3.90 (s, 3H), 3.77/3.68 (t, 2H), 3.75 (s, 2H), 3.14 (s, 3H), 2.82/2.74 (t, 2H), 2.40 (s, 3H), 0.89 (s, 9H), 0.12 (s, 6H).

HRMS-ESI (m/z) [M+H]⁺ calcd for C₅₁H₅₉FN₅O₈Si: 916.4111, found 916.4118.

### Step E: N-(3-cyano-4-methoxy-phenyl)-5-[2-[2-[2-fluoro-4-(2-morpholinoethoxy)phenyl]acetyl]-7-[(3S)-3-[[isopropyl(methyl)amino]methyl]-3,4-dihydro-1H-isoquinoline-2-carbonyl]-3,4-dihydro-1H-isoquinolin-6-yl-N-(4-hydroxyphenyl)-1,2-dimethyl-pyrrole-3-carboxamide

Using General Procedure 4c, starting from the product of Step D (90 mg, 0.098 mmol) as the appropriate acid and the compound obtained in Step B of Example 91 (26 mg, 1.2 eq.) as the appropriate amine, afforded the title product (70 mg, 71%).

HRMS-ESI (m/z) [M+H]⁺ calcd for C₅₉H₆₅FN₇O₇: 1002.4924, found 1002.4923.

### Example 180: 5-[7-[(3S)-3-(2-azaspiro[3.3]heptan-2-ylmethyl)-3,4-dihydro-1H-isoquinoline-2-carbonyl]-2-[2-[4-(2-tetrahydropyran-4-ylethoxy)phenyl]acetyl]-3,4-dihydro-1H-isoquinolin-6-yl]-N-[5-cyano-1-(difluoromethyl)-2-methyl-pyrrol-3-yl]-N-(4-hydroxyphenyl)-1,2-dimethyl-pyrrole-3-carboxamide

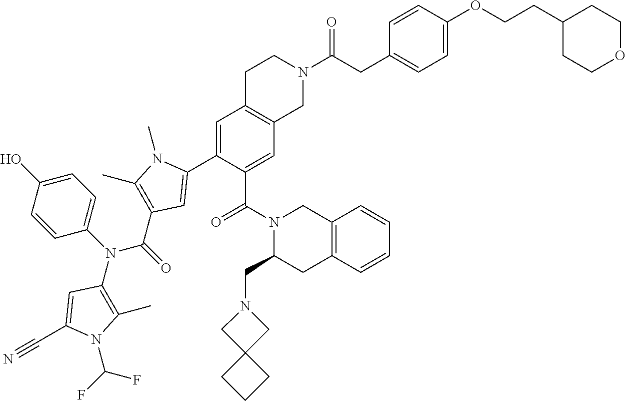 [SMILES: Cc1c(C(=O)N(c2ccc(O)cc2)c2cc(C#N)n(C(F)F)c2C)cc(-c2cc3c(cc2C(=O)N2Cc4ccccc4C[C@H]2CN2CC4(CCC4)C2)CN(C(=O)Cc2ccc(OCCC4CCOCC4)cc2)CC3)n1C]

### Step A: 4-[4-[tert-butyl(dimethyl)silyl]oxyanilino]-1-(difluoromethyl)-5-methyl-pyrrole-2-carbonitrile

Using General procedure 1b and 4-bromo-]-(difluoromethyl)-5-methyl-pyrrole-2-carbonitrile (1.26 g, 5.36 mmol) as the appropriate aryl bromide and Preparation IIa (1.32 g, 1.1 eq.) as the appropriate aniline, afforded the title product (1.36 g, 67%).

¹H NMR (500 MHz, DMSO-d6) δ ppm 7.79 (t, 1H), 7.16 (s, 1H), 7.12 (s, 1H), 6.64 (d, 2H), 6.56 (d, 2H), 2.24 (s, 3H), 0.93 (s, 9H), 0.12 (s, 6H); ¹³C NMR (125 MHz, DMSO-d6) δ ppm 147.4, 141.3, 128.3, 127.2, 121.2, 120.7, 114.9, 113.0, 109.7, 97.7, 26.1, 18.3, 9.7, −4.0.

HRMS-ESI (m/z) [M+H]⁺ calcd for C₁₉H₂₆F₂N₃OSi: 378.1808, found 378.1807.

### Step B: 02-benzyl 07-tert-butyl 6-[4-[[4-[tert-butyl(dimethyl)silyl]oxyphenyl]-[5-cyano-1-(difluoromethyl)-2-methyl-pyrrol-3-yl]carbamoyl]-1,5-dimethyl-pyrrol-2-yl]-3,4-dihydro-1H-isoquinoline-2,7-dicarboxylate

Using General procedure 2b, starting from the product of Step B of Preparation IX (1.05 g, 2.08 mmol) as the appropriate acid and the product of Step A (0.943 g, 1.2 eq.) as the appropriate aniline, the title compound was obtained (0.93 g, 51%).

¹H NMR (500 MHz, DMSO-d6) δ ppm 7.82 (t, 1H), 7.60 (s, 1H), 7.40-7.30 (m, 5H), 7.20 (s, 1H), 7.06 (d, 2H), 6.87 (s, 1H), 6.76 (d, 2H), 5.14/5.06 (s/s, 1H), 5.13 (s, 2H), 4.67/4.61 (s/s, 2H), 3.64 (br, 2H), 3.14 (s, 3H), 2.78 (t, 2H), 2.42 (s, 3H), 2.19 (s, 3H), 1.28 (s, 9H), 0.84 (s, 9H), 0.08 (s, 6H); ¹³C NMR (125 MHz, DMSO-d6) δ ppm 132.8, 128.7, 128.1, 123.8, 120.8, 109.6, 108.4, 66.8, 45.4, 41.5/41.1, 31.4, 28.5/28.4, 27.8, 26, 12.0, 10.0, −4.2.

HRMS-ESI (m/z) [M+H]⁺ calcd for C₄₈H₅₆F₂N₅O₆Si: 864.3962, found 864.3965.

### Step C. tert-butyl 6-[4-[[4-[tert-butyl(dimethyl)silyl]oxyphenyl]-[5-cyano-1-(difluoromethyl)-2-methyl-pyrrol-3-yl]carbamoyl]-1,5-dimethyl-pyrrol-2-yl]-1,2,3,4-tetrahydroisoquinoline-7-carboxylate

According to General procedure 6b the product from Step B (930 mg, 1.07 mmol, 1.0 eq.) in EtOAc (12 mL) was hydrogenated using 30 mg Pd catalyst (10% on charcoal) affording the title product (0.629 g, 80%).

¹H NMR (500 MHz, DMSO-d6) δ ppm 7.82 (t, 1H), 7.44 (s, 1H), 7.21 (s, 1H), 7.07 (m, 2H), 6.77 (m, 2H), 6.75 (s, 1H), 5.06 (s, 1H), 3.89 (s, 2H), 3.13 (s, 3H), 2.95 (t, 2H), 2.67 (t, 2H), 2.42 (s, 3H), 2.18 (s, 3H), 1.27 (s, 9H), 0.87 (s, 9H), 0.11 (s, 6H).

HRMS-ESI (m/z) [M+H]⁺ calcd for C₄₀H₅₀F₂N₅O₄Si: 730.3595, found 730.3599.

### Step D: 6-[4-[[4-[tert-butyl(dimethyl)silyl]oxyphenyl]-[5-cyano-1-(difluoromethyl)-2-methyl-pyrrol-3-yl]carbamoyl]-1,5-dimethyl-pyrrol-2-yl]-2-[2-[4-(2-tetrahydropyran-4-ylethoxy)phenyl]acetyl]-3,4-dihydro-1H-isoquinoline-7-carboxylic acid

Using General procedure 4c without the deprotection step, starting from Preparation Vc (45 mg, 1.1 eq.) as the appropriate carboxylic acid and the product from Step C (110 mg, 0.151 mmol) as the appropriate amine afforded the title compound after deprotection using General Procedure 3a (31 mg, 22%).

¹H NMR (500 MHz, DMSO-d6) δ ppm 12.65 (brs, 1H), 7.77/7.76 (t/t, 1H), 7.68-6.68 (m, 11H), 5.14-5.08 (s, 1H), 4.78-1.16 (m, 21H), 3.12/3.11 (s, 3H), 2.40-2.12 (s+s, 6H), 0.92-0.83 (s, 9H), 0.14-−0.05 (s, 6H).

HRMS-ESI (m/z) [M+H]⁺ calcd for C₅₁H₆₀F₂N₅O₇Si: 920.4224, found 920.4223.

### Step E: tert-butyl (3S)-3-(2-azaspiro[3.3]heptane-2-carbonyl)-3,4-dihydro-1H-isoquinoline-2-carboxylate

Using General procedure 5a, starting from (3S)-2-tert-butoxycarbonyl-3,4-dihydro-]H-isoquinoline-3-carboxylic acid (1.70 g, 6.1 mmol) and 2-azaspiro[3.3]heptane oxalic acid salt (1.00 g, 1.2 eq) the title product (2.25 mg, 99%) was obtained.

¹H NMR (500 MHz, DMSO-d6) δ ppm 7.28-7.06 (m, 4H), 4.74/4.33 (br/t, 1H), 4.68-4.20 (m, 2H), 4.20-3.68 (m, 4H), 3.11-2.76 (m, 2H), 2.21-2.01 (br., 4H), 1.83-1.68 (br., 2H); ¹³C NMR (125 MHz, DMSO-d6) δ ppm 62.7/60.6, 52.2/50.8, 44.8/44.1, 32.8, 30.9/30.4, 16.0.

HRMS-ESI (m/z) [M+Na]⁺ calcd for C₂₁H₂₈N₂O₃Na: 379.1992, found 379.1995.

### Step F: 2-azaspiro[3.3]heptan-2-yl-[(3S)-1,2,3,4-tetrahydroisoquinolin-3-yl]methanone

Using General procedure 6c, starting from the product of Step E (2.25 mg, 6.31 mmol), the title product (1.72 mg, 83%) was obtained.

¹H NMR (500 MHz, DMSO-d6) δ ppm 9.80/9.47 (brs+brs, 2H), 7.28-7.22 (m, 4H), 4.32 (dd, 1H), 4.29/4.26 (d+d, 2H), 4.28/4.27/3.99/3.95 (d+d/d+d, 4H), 3.22/2.92 (dd+dd, 2H), 2.19/2.16 (m/m, 4H), 1.79 (m, 2H); ¹³C NMR (125 MHz, DMSO-d6) δ ppm 62.5/60.7, 51.7, 44.3, 32.9/32.6, 28.5, 16.0.

HRMS-ESI (m/z) [M+Na]⁺ calcd for C₁₆H₂₀N₂ONa: 279.1468, found 279.1469.

### Step G: (3S)-3-(2-azaspiro[3.3]heptan-2-ylmethyl)-1,2,3,4-tetrahydroisoquinoline

Using General procedure 6a, starting from the product of Step F (1.72 g, 4.99 mmol), the title compound (0.888 g, 68%) was obtained.

¹H NMR (400 MHz, DMSO-d6) δ ppm 7.11-6.95 (m, 4H), 3.86 (brs, 2H), 3.13/3.09 (d+d, 4H), 2.64 (m, 1H), 2.61/2.35 (m+m, 2H), 2.39/2.33 (dd+dd, 2H), 2.28 (brs, 1H), 2.03 (t, 4H), 1.75 (m, 2H).

HRMS-ESI (m/z) [M+H]⁺ calcd for C₁₆H₂₃N₂: 243.1856, found 243.1855.

### Step H. 5-[7-[(3S)-3-(2-azaspiro[3.3]heptan-2-ylmethyl)-3,4-dihydro-1H-isoquinoline-2-carbonyl]-2-[2-[4-(2-tetrahydropyran-4-ylethoxy)phenyl]acetyl]-3,4-dihydro-1H-isoquinolin-6-yl]-N-[5-cyano-1-(difluoromethyl)-2-methyl-pyrrol-3-yl]-N-(4-hydroxyphenyl)-1,2-dimethyl-pyrrole-3-carboxamide

Using General Procedure 4c, starting from the product of Step D (30 mg, 0.033 mmol) as the appropriate acid and the compound obtained in Step G (9.1 mg, 1.1 eq.) as the appropriate amine, afforded the title product (23 mg, 68%).

HRMS-ESI (m/z) [M+H]⁺ calcd for C₆₁H₆₆F₂N₇O₆: 1030.5037, found 1030.5044.

### Example 181: 5-[7-[(3S)-3-(2-azaspiro[3.3]heptan-2-ylmethyl)-3,4-dihydro-1H-isoquinoline-2-carbonyl]-2-[2-[2-fluoro-4-(2-morpholinoethoxy)phenyl]acetyl]-3,4-dihydro-1H-isoquinolin-6-yl]-N-(3-cyano-4-methoxy-phenyl)-N-(4-hydroxyphenyl)-1,2-dimethyl-pyrrole-3-carboxamide

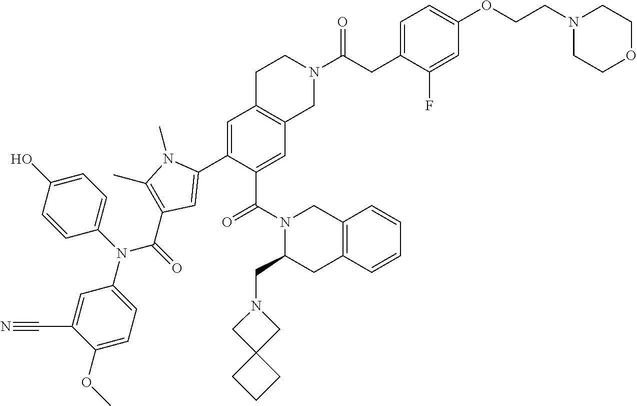 [SMILES: COc1ccc(N(C(=O)c2cc(-c3cc4c(cc3C(=O)N3Cc5ccccc5C[C@H]3CN3CC5(CCC5)C3)CN(C(=O)Cc3ccc(OCCN5CCOCC5)cc3F)CC4)n(C)c2C)c2ccc(O)cc2)cc1C#N]

### Step A: 02-benzyl 07-tert-butyl 6-[4-[[4-[tert-butyl(dimethyl)silyl]oxyphenyl]-(3-cyano-4-methoxy-phenyl)carbamoyl]-1,5-dimethyl-pyrrol-2-yl]-3,4-dihydro-1H-isoquinoline-2,7-dicarboxylate

Using General procedure 2b, starting from the product of Step B of Preparation IX (700 mg, 1.387 mmol) as the appropriate acid and the product of Step A in Example 81 (590 mg, 1.2 eq.) as the appropriate aniline, the title compound was obtained (860 mg, 74%).

¹H NMR (500 MHz, DMSO-d6) δ ppm 7.60 (s, 1H), 7.55 (d, 1H), 7.46 (dd, 1H), 7.42-7.29 (m, 5H), 7.21 (d, 1H), 7.09 (dm, 2H), 6.86 (s, 1H), 6.77 (dm, 2H), 5.12 (s, 2H), 5.03 (s, 1H), 4.66/4.60 (br/br., 2H), 3.89 (s, 3H), 3.63 (br., 2H), 3.14 (s, 3H), 2.78 (t, 2H), 2.43 (s, 3H), 1.28 (s, 9H), 0.83 (s, 9H), 0.07 (s, 6H); ¹³C NMR (125 MHz, DMSO-d6) δ ppm 166.5, 166.3, 159.0, 155.1, 154.0, 138.4, 138.2, 137.3, 134.7, 132.8, 132.5, 129.8, 128.1, 120.9, 116.4, 113.3, 108.8, 100.7, 66.8, 57.0, 45.5, 41.4/41.1, 31.5, 28.6/28.4, 27.8, 25.9, 12.2, −4.2.

HRMS-ESI (m/z) [M+H]⁺ calcd for C₄₉H₅₇N₄O₇Si:: 841.3991, found 841.3995.

### Step B: tert-butyl 6-[4-[[4-[tert-butyl(dimethyl)silyl]oxyphenyl]-(3-cyano-4-methoxy-phenyl)carbamoyl]-1,5-dimethyl-pyrrol-2-yl]-1,2,3,4-tetrahydroisoquinoline-7-carboxylate

According to General procedure 6b the product from Step A (850 mg, 1.01 mmol) in methanol (12 mL) was hydrogenated using 22 mg Pd catalyst (10% on charcoal) affording the title product (0.693 g, 97%).

¹H NMR (500 MHz, DMSO-d6) δ ppm 7.54 (d, 1H), 7.46 (dd, 1H), 7.42 (s, 1H), 7.21 (d, 1H), 7.09 (dm, 2H), 6.78 (dm, 2H), 6.72 (s, 1H), 5.02 (s, 1H), 3.89 (s, 3H), 3.85 (s, 2H), 3.12 (s, 3H), 2.91 (t, 2H), 2.64 (t, 2H), 2.43 (s, 3H), 1.27 (s, 9H), 0.86 (s, 9H), 0.09 (s, 6H); ¹³C NMR (125 MHz, DMSO-d6) δ ppm 166.5, 166.4, 159.0, 154.0, 138.3, 138.2, 134.7, 133.2, 132.5, 129.8, 127.9, 120.9, 116.4, 113.3, 108.6, 100.7, 57.0, 47.8, 43.3, 31.4, 29.2, 27.9, 25.9, 12.2, −4.2.

HRMS-ESI (m/z) [M+H]⁺ calcd for C₄₁H₅₁N₄O₅Si: 707.3623, found 707.3623.

### Step C: 6-[4-[[4-[tert-butyl(dimethyl)silyl]oxyphenyl]-(3-cyano-4-methoxy-phenyl)carbamoyl]-1,5-dimethyl-pyrrol-2-yl]-2-[2-[2-fluoro-4-(2-morpholinoethoxy)phenyl]acetyl]-3,4-dihydro-1H-isoquinoline-7-carboxylic acid

Using General procedure 4c without the deprotection step, starting from Preparation Vb (332 mg, 1.2 eq.) as the appropriate carboxylic acid and the product from Step B (690 mg, 0.976 mmol) as the appropriate amine afforded the title compound after deprotection using General Procedure 3a (645 mg, 72%).

¹H NMR (500 MHz, DMSO-d6) δ ppm 12.63 (brs, 1H), 7.71-6.67 (m, 12H), 5.06 (s, 1H), 4.81/4.66 (s, 2H), 4.60-2.20 (brm, 12H), 3.90 (s, 3H), 3.77/3.68 (t, 2H), 3.75 (s, 2H), 3.14 (s, 3H), 2.82/2.74 (t, 2H), 2.40 (s, 3H), 0.89 (s, 9H), 0.12 (s, 6H).

HRMS-ESI (m/z) [M+H]⁺ calcd for C₅₁H₅₉FN₅O₈Si: 916.4111, found 916.4118.

### Step D: 5-[7-[(3S)-3-(2-azaspiro[3.3]heptan-2-ylmethyl)-3,4-dihydro-1H-isoquinoline-2-carbonyl]-2-[2-[2-fluoro-4-(2-morpholinoethoxy)phenyl]acetyl]-3,4-dihydro-1H-isoquinolin-6-yl]-N-(3-cyano-4-methoxy-phenyl)-N-(4-hydroxyphenyl)-1,2-dimethyl-pyrrole-3-carboxamide

Using General Procedure 4c, starting from the product of Step C (90 mg, 0.098 mmol) as the appropriate acid and the compound obtained in Step G in Example 180 (29 mg, 1.2 eq.) as the appropriate amine, afforded the title product (54 mg, 54%).

HRMS-ESI (m/z) [M+H]⁺ calcd for C₆₁H₆₅FN₇O₇: 1026.4924, found 1026.4922.

### Example 182: N-(3-cyano-4-methoxy-phenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

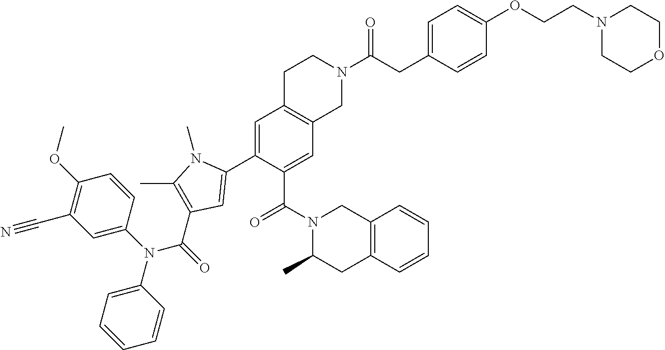 [SMILES: COc1ccc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cc1C#N]

### Step A: 5-anilino-2-methoxy-benzonitrile

Using General procedure 1b and 5-bromo-2-methoxy-benzonitrile (1.20 g, 5.66 mmol) as the appropriate aryl bromide and aniline (791 mg, 1.5 eq.) afforded the title product (1.04 g, 81%).

¹H NMR (500 MHz, DMSO-d6) δ ppm 8.14 (s, 1H), 7.37 (dd, 1H), 7.30 (d, 1H), 7.22 (t, 2H), 7.17 (d, 1H), 6.98 (d, 2H), 6.81 (t, 1H), 3.86 (s, 3H); ¹³C NMR (125 MHz, DMSO-d6) δ ppm 155.4, 144.1, 137.6, 129.8, 125.2, 121.9, 120.2, 117.0, 116.5, 114.0, 101.0, 56.8.

HRMS-ESI (m/z) [M+H]⁺ calcd for C₁₄H₁₃N₂O: 225.1022, found 225.1023.

### Step B: N-(3-cyano-4-methoxy-phenyl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.63-6.78 (m, 14H), 5.60-4.94 (s, 1H), 5.25-2.02 (m, 12H), 3.93-3.78 (s, 3H), 3.39-3.03 (s, 3H), 2.44-2.04 (s, 3H), 1.09-0.43 (d, 3H).

HRMS-ESI (m/z) [M+H]⁺ calcd for C₄₁H₄₀N₅O₃: 650.3126, found 650.3128.

### Step C. N-(3-cyano-4-methoxy-phenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without the deprotection step, starting from Preparation Va as the appropriate acid (56 mg, 1.5 eq.) and the product of Step B (85 mg, 0.131 mmol) as the appropriate amine, the desired product was obtained (74 mg, 62%).

HRMS-ESI (m/z) [M+H]⁺ calcd for C₅₅H₅₇N₆O₆: 897.4334, found 897.4329.

### Example 183: N-(5-fluoro-6-methoxy-3-pyridyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

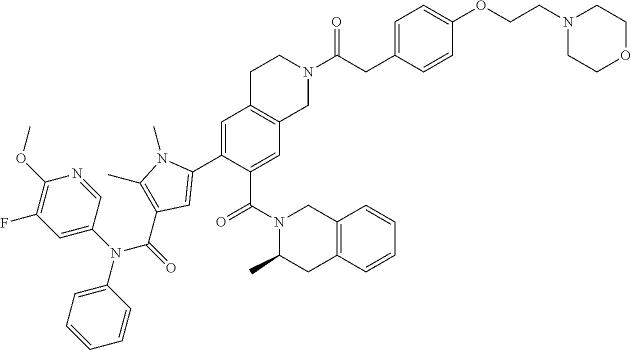 [SMILES: COc1ncc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cc1F]

### Step A: 5-fluoro-6-methoxy-N-phenyl-pyridin-3-amine

Using General procedure 1b and 5-bromo-3-fluoro-2-methoxy-pyridine (1.16 g, 5.63 mmol) as the appropriate aryl bromide and aniline (787 mg, 1.5 eq.) afforded the title product (548 mg, 43%).

¹H NMR (500 MHz, DMSO-d6) δ ppm 8.15 (s, 1H), 7.78 (d, 1H), 7.45 (dd, 1H), 7.22 (t, 2H), 6.97 (d, 2H), 6.81 (t, 1H), 3.89 (s, 3H); ¹³C NMR (125 MHz, DMSO-d6) δ ppm 147.3, 146.8, 144.2, 135.3, 131.3, 129.8, 120.1, 116.0, 116.0, 53.8.

HRMS-ESI (m/z) [M+H]⁺ calcd for C₁₂H₁₂FN₂O: 219.0928, found 219.0928.

### Step B: N-(5-fluoro-6-methoxy-3-pyridyl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.89-6.75 (m, 13H), 5.65-5.01 (s, 1H), 5.27-1.99 (m, 12H), 4.00-3.75 (s, 3H), 3.40-2.62 (s, 3H), 2.42-2.02 (s, 3H), 1.10-0.39 (d, 3H).

HRMS-ESI (m/z) [M+H]⁺ calcd for C₃₉H₃₉FN₅O₃: 644.3031, found 644.3032.

### Step C: N-(5-fluoro-6-methoxy-3-pyridyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without the deprotection step, starting from Preparation Va as the appropriate acid (58 mg, 1.5 eq.) and the product of Step B (85 mg, 0.132 mmol) as the appropriate amine, the desired product was obtained (69 mg, 58%).

HRMS-ESI (m/z) [M+H]⁺ calcd for C₅₃H₅₆FN₆O₆: 891.4240, found 891.4246.

### Example 184: 1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-(1-methylpyrazol-4-yl)-N-phenyl-pyrrole-3-carboxamide

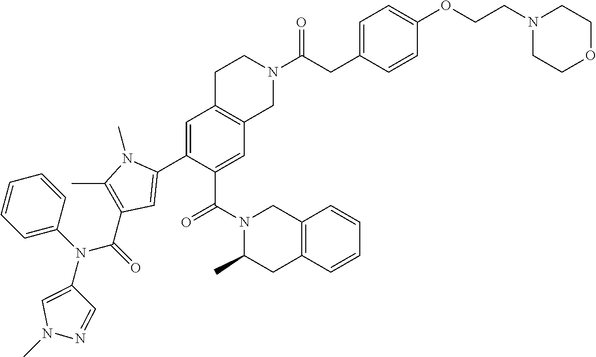 [SMILES: Cc1c(C(=O)N(c2ccccc2)c2cnn(C)c2)cc(-c2cc3c(cc2C(=O)N2Cc4ccccc4C[C@H]2C)CN(C(=O)Cc2ccc(OCCN4CCOCC4)cc2)CC3)n1C]

### Step A: 1-methyl-N-phenyl-pyrazol-4-amine

Using General procedure 1b and 4-bromo-1-methyl-pyrazole (600 mg, 3.73 mmol) as the appropriate aryl bromide and aniline (521 mg, 1.5 eq.) afforded the title product (548 mg, 43%).

¹H NMR (500 MHz, DMSO-d6) δ ppm 7.62 (d, 1H), 7.48 (s, 1H), 7.28 (d, 1H), 7.10 (t, 2H), 6.75 (d, 2H), 6.60 (t, 1H), 3.79 (s, 3H); ¹³C NMR (125 MHz, DMSO-d6) δ ppm 147.2, 133.0, 129.5, 125.4, 123.0, 117.3, 113.2, 39.2.

HRMS-ESI (m/z) [M+H]⁺ calcd for C₁₀H₁₂N₃: 174.1026, found 174.1026.

### Step B: 1,2-dimethyl-5-[7-[(3R)-3-methyl-3,4-dihydro-1H-isoquinoline-2-carbonyl]-1,2,3,4-tetrahydroisoquinolin-6-yl]-N-(1-methylpyrazol-4-yl)-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 8.52 (brs, 2H), 7.73-6.79 (m, 13H), 5.55-5.00 (s, 1H), 5.3-1.94 (m, 11H), 3.81-3.05 (s, 6H), 2.44-2.02 (s, 3H), 1.07-0.42 (d, 3H).

HRMS-ESI (m/z) [M+H]⁺ calcd for C₃₇H₃₉N₆O₂: 599.3129, found 599.3131.

### Step C. 1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-(1-methylpyrazol-4-yl)-N-phenyl-pyrrole-3-carboxamide

Using General procedure 4c without the deprotection step, starting from Preparation Va as the appropriate acid (69 mg, 1.1 eq.) and the product of Step B (138 mg, 0.230 mmol) as the appropriate amine, the desired product was obtained (90 mg, 46%).

HRMS-ESI (m/z) [M+H]⁺ calcd for C₅₁H₅₆N₇O₅: 846.4337, found 846.4339.

### Example 185: N-(5-cyano-1,2-dimethyl-pyrrol-3-yl)-N-(4-fluorophenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

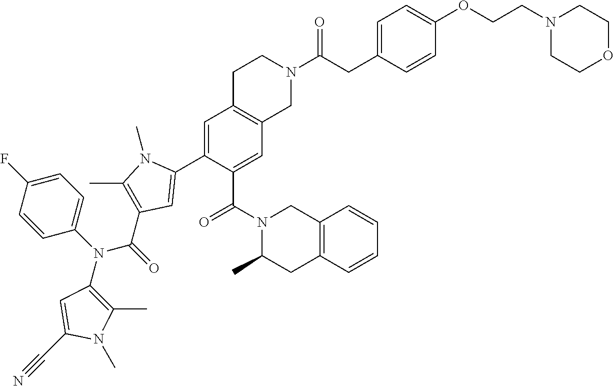 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(F)cc2)cc(C#N)n1C]

### Step A: 4-(4-fluoroanilino)-1,5-dimethyl-pyrrole-2-carbonitrile

Using General procedure 1b and 4-bromo-1,5-dimethyl-pyrrole-2-carbonitrile (500 mg, 2.51 mmol) as the appropriate aryl bromide and 4-fluoroaniline (307 mg, 1.1 eq.) as the appropriate aniline afforded the title product (406 mg, 71%).

¹H NMR (500 MHz, DMSO-d6) δ ppm 7.15 (s, 1H), 6.91 (t, 2H), 6.77 (s, 1H), 6.57 (dd, 2H), 3.62 (s, 3H), 2.08 (s, 3H); ¹³C NMR (125 MHz, DMSO-d6) δ ppm 155.1, 144.8, 131.5, 124.0, 115.8, 115.7, 114.8, 113.9, 100.2, 33.2, 9.9.

HRMS-ESI (m/z) [M+H]⁺ calcd for C₁₃H₁₃FN₃: 230.1088, found 230.1087.

### Step B: N-(5-cyano-1,2-dimethyl-pyrrol-3-yl)-N-(4-fluorophenyl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

The title compound was obtained using the method described in Preparation VIIb and the product from Step A as the appropriate aniline for the amide formation.

¹H NMR (500 MHz, DMSO-d6) δ ppm 7.32-6.47 (m, 11H), 5.57-5.10 (s, 1H), 5.36-1.90 (m, 12H), 3.70-2.97 (s, 6H), 2.47-1.80 (s, 6H), 1.11-0.40 (d, 3H).

HRMS-ESI (m/z) [M+H]⁺ calcd for C₄₀H₄₀FN₆O₂: 655.3191, found 655.3192.

### Step C. N-(5-cyano-1,2-dimethyl-pyrrol-3-yl)-N-(4-fluorophenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

Using General procedure 4c without the deprotection step, starting from Preparation Va as the appropriate acid (66 mg, 1.5 eq.) and the product of Step B (108 mg, 0.165 mmol) as the appropriate amine, the desired product was obtained (112 mg, 75%).

HRMS-ESI (m/z) [M+H]⁺ calcd for C₅₄H₅₇FN₇O₅: 902.4400, found 902.4407.

### Example 186: N-[1-(difluoromethyl)pyrazol-4-yl]-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

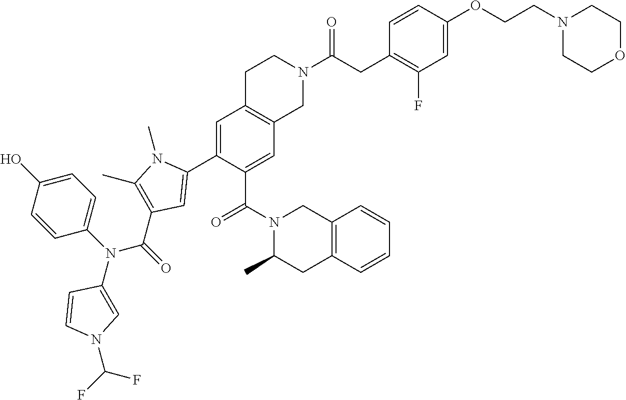 [SMILES: Cc1c(C(=O)N(c2ccc(O)cc2)c2ccn(C(F)F)c2)cc(-c2cc3c(cc2C(=O)N2Cc4ccccc4C[C@H]2C)CN(C(=O)Cc2ccc(OCCN4CCOCC4)cc2F)CC3)n1C]

### Step A: N-[4-[tert-butyl(dimethyl)silyl]oxyphenyl]-1-(difluoromethyl)pyrazol-4-amine

Using General procedure 1b, starting from 4-bromo-]-(difluoromethyl)pyrazole (1.00 g, 5.08 mmol) as the appropriate aryl-bromide and Preparation IIa (1.19 g, 1.05 eq.) as the appropriate aniline, afforded the title compound (1.19 g, 69%).

¹H NMR (500 MHz, DMSO-d6) δ ppm 7.99 (s, 1H), 7.72 (s, 1H), 7.67 (t, 1H), 7.63 (s, 1H), 6.79 (d, 2H), 6.70 (d, 2H), 0.93 (s, 9H), 0.14 (s, 6H); ¹³C NMR (125 MHz, DMSO-d6) δ ppm 147.7, 139.7, 136.3, 129.6, 120.8, 115.5, 115.4, 111.3, 26.1, 18.4, −4.1.

HRMS-ESI (m/z) [M+H]⁺ calcd for C₁₆H₂₄F₂N₃OSi: 340.1651, found 340.1650.

### Step B: N-[4-[tert-butyl(dimethyl)silyl]oxyphenyl]-N-[1-(difluoromethyl)pyrazol-4-yl]-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

The title compound was obtained using the method described in Preparation VIIb and the product from Step A as the appropriate aniline for the amide formation.

¹H NMR (500 MHz, DMSO-d6) δ ppm 8.24-8.02 (s, 1H), 7.73 (t, 1H), 7.54-7.37 (s, 1H), 7.26-6.55 (m, 10H), 5.30-4.67 (s, 1H), 5.26-2.09 (m, 12H), 3.35-3.02 (s, 3H), 2.46-2.08 (s, 3H), 1.04-0.52 (d, 3H), 0.86 (s, 9H), 0.17-0.04 (s, 6H).

HRMS-ESI (m/z) [M+H]⁺ calcd for C₄₃H₅₁F₂N₆O₃Si: 765.3754, found 765.3756.

### Step C: N-[1-(difluoromethyl)pyrazol-4-yl]-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

Using General procedure 4c, starting from Preparation Vb as the appropriate acid (36 mg, 1.2 eq.) and the product of Step B (80 mg, 0.105 mmol) as the appropriate amine, the desired product was obtained (42 mg, 44%).

HRMS-ESI (m/z) [M+H]⁺ calcd for C₅₁H₅₃F₃N₇O₆: 916.4004, found 916.4007.

### Example 187: N-(1,3-dimethylpyrazol-4-yl)-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

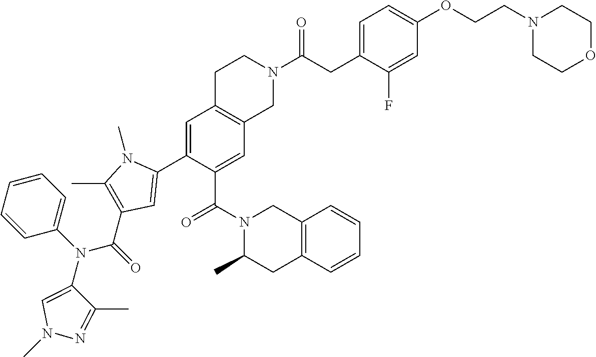 [SMILES: Cc1nn(C)cc1N(C(=O)c1cc(-c2cc3c(cc2C(=O)N2Cc4ccccc4C[C@H]2C)CN(C(=O)Cc2ccc(OCCN4CCOCC4)cc2F)CC3)n(C)c1C)c1ccccc1]

### Step A: 1,3-dimethyl-N-phenyl-pyrazol-4-amine

Using General procedure 1b and 4-bromo-1,3-dimethyl-pyrazole (300 mg, 1.714 mmol) as the appropriate aryl bromide and aniline (239 mg, 1.5 eq.) afforded the title product (210 mg, 59%).

¹H NMR (500 MHz, DMSO-d6) δ ppm 7.54 (s, 1H), 7.07 (m, 2H), 7.04 (s, 1H), 6.60 (m, 2H), 6.56 (m, 1H), 3.72 (s, 3H), 1.98 (s, 3H); ¹³C NMR (125 MHz, DMSO-d6) δ ppm 148.2, 143.0, 129.4, 126.3, 121.9, 117.0, 113.0, 39.0, 11.4.

HRMS-ESI (m/z) [M+H]⁺ calcd for C₁₁H₁₄N₃: 188.1182, found 188.1184.

### Step B: N-(1,3-dimethylpyrazol-4-yl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.64-7.43 (s, 1H), 7.37-6.83 (m, 11H), 5.68-5.22 (s, 1H), 5.29-1.95 (m, 12H), 3.76-3.03 (s, 6H), 2.43-1.57 (s, 6H), 1.07-0.41 (d, 3H).

HRMS-ESI (m/z) [M+H]⁺ calcd for C₃₈H₄₁N₆O₂: 613.3286, found 613.3289.

### Step C: N-(1,3-dimethylpyrazol-4-yl)-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

Using General procedure 4c without the deprotection step, starting from Preparation Vb as the appropriate acid (69 mg, 1.5 eq.) and the product of Step B (100 mg, 0.163 mmol) as the appropriate amine, the desired product was obtained (75 mg, 52%).

HRMS-ESI (m/z) [M+H]⁺ calcd for C₅₂H₅₇FN₇O₅: 878.4400, found 878.4399.

### Example 188: N-(1,3-dimethylpyrazol-4-yl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

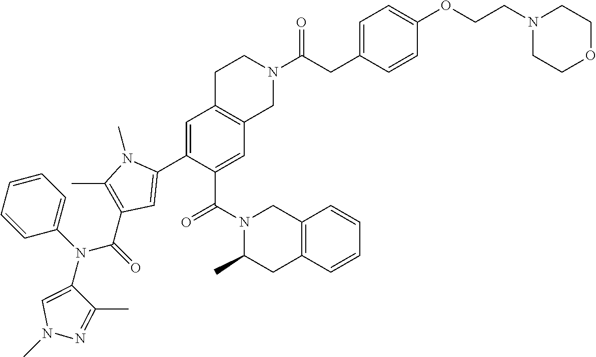 [SMILES: Cc1nn(C)cc1N(C(=O)c1cc(-c2cc3c(cc2C(=O)N2Cc4ccccc4C[C@H]2C)CN(C(=O)Cc2ccc(OCCN4CCOCC4)cc2)CC3)n(C)c1C)c1ccccc1]

Using General procedure 4c without the deprotection step, starting from Preparation Va as the appropriate acid (65 mg, 1.5 eq.) and the product of Step B in Example 187 (100 mg, 0.163 mmol) as the appropriate amine, the desired product was obtained (57 mg, 40%).

HRMS-ESI (m/z) [M+H]⁺ calcd for C₅₂H₅₈N₇O₅: 860.4494, found 860.4497.

### Example 189: N-(5-cyano-1,2-dimethyl-pyrrol-3-yl)-5-[2-(2-methoxyacetyl)-7-[(3S)-3-(1-piperidylmethyl)-3,4-dihydro-1H-isoquinoline-2-carbonyl]-3,4-dihydro-1H-isoquinolin-6-yl]-1,2-dimethyl-N-phenyl-pyrrole-3-carboxamide

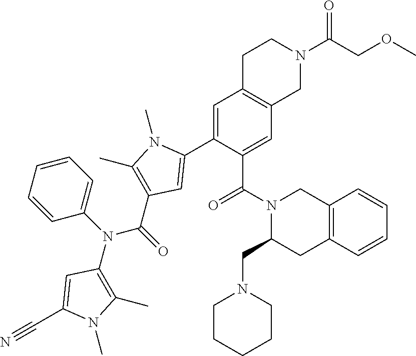 [SMILES: COCC(=O)N1CCc2cc(-c3cc(C(=O)N(c4ccccc4)c4cc(C#N)n(C)c4C)c(C)n3C)c(C(=O)N3Cc4ccccc4C[C@H]3CN3CCCCC3)cc2C1]

Using General procedure 4c without the deprotection step, starting from 2-methoxyacetic acid as the appropriate acid (19 mg, 1.5 eq.) and the product of Example 92, Step C (100 mg, 0.139 mmol) as the appropriate amine, the desired product was obtained (64 mg, 58%).

HRMS-ESI (m/z) [M+H]⁺ calcd for C₄₈H₅₄N₇O₄: 792.4232, found 792.4236.

### Example 190: N-[1-(difluoromethyl)pyrazol-4-yl]-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

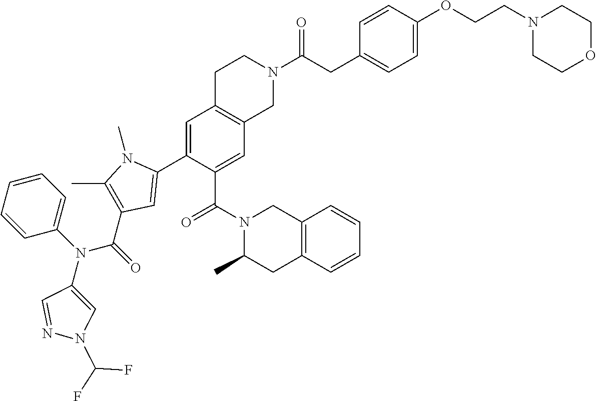 [SMILES: Cc1c(C(=O)N(c2ccccc2)c2cnn(C(F)F)c2)cc(-c2cc3c(cc2C(=O)N2Cc4ccccc4C[C@H]2C)CN(C(=O)Cc2ccc(OCCN4CCOCC4)cc2)CC3)n1C]

### Step A: 1-(difluoromethyl)-N-phenyl-pyrazol-4-amine

Using General procedure 1b and 4-bromo-]-(difluoromethyl)pyrazole (1.00 g, 5.077 mmol) as the appropriate aryl bromide and aniline (0.496 g, 1.05 eq.) afforded the title product (855 mg, 81%).

¹H NMR (500 MHz, DMSO-d6) δ ppm 8.09 (s, 1H), 7.97 (s, 1H), 7.7 (t, 1H), 7.69 (s, 1H), 7.18 (m, 2H), 6.87 (dm, 2H), 6.71 (tm, 1H); ¹³C NMR (125 MHz, DMSO-d6) δ ppm 145.4, 136.8, 129.7, 128.5, 118.5, 116.8, 114.1, 111.2.

HRMS-ESI (m/z) [M+H]⁺ calcd for C₁₀H₁₀F₂N₃: 210.0837, found 210.0838.

### Step B: N-[1-(difluoromethyl)pyrazol-4-yl]-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 8.19-7.98 (s, 1H), 7.71 (t, 1H), 7.61-6.75 (m, 12H), 5.41-4.85 (s, 1H), 5.27-1.92 (m, 12H), 3.37-3.01 (s, 3H), 2.48-2.04 (s, 3H), 1.07-0.42 (d, 3H).

HRMS-ESI (m/z) [M+H]⁺ calcd for C₃₇H₃₇F₂N₆O₂: 635.2938, found 635.2938.

### Step C. N-[1-(difluoromethyl)pyrazol-4-yl]-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without the deprotection step, starting from Preparation Va as the appropriate acid (35 mg, 1.2 eq.) and the product of Step B (70 mg, 0.110 mmol) as the appropriate amine, the desired product was obtained (65 mg, 67%).

HRMS-ESI (m/z) [M+H]⁺ calcd for C₅₁H₅₄F₂N₇O₅: 882.4149, found 882.4153.

### Example 191: N-(6-methoxy-3-pyridyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

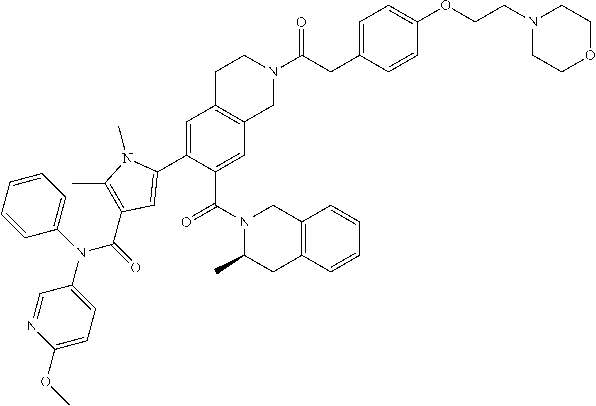 [SMILES: COc1ccc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cn1]

### Step A: 6-methoxy-N-phenyl-pyridin-3-amine

Using General procedure 1b and 5-bromo-2-methoxy-pyridine (300 mg, 1.596 mmol) as the appropriate aryl bromide and aniline (223 mg, 1.5 eq.) afforded the title product (264 mg, 64%).

¹H NMR (500 MHz, DMSO-d6) δ ppm 7.96 (d, 1H), 7.93 (s, 1H), 7.51 (dd, 1H), 7.17 (m, 2H), 6.89 (m, 2H), 6.77 (d, 1H), 6.74 (m, 1H), 3.81 (s, 3H); ¹³C NMR (125 MHz, DMSO-d6) δ ppm 159.0, 145.3, 137.9, 134.1, 132.1, 129.7, 119.2, 115.2, 111.0, 53.5.

HRMS-ESI (m/z) [M+H]⁺ calcd for C₁₂H₁₃N₂O: 201.1022, found 201.1023.

### Step B: N-(6-methoxy-3-pyridyl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.97-7.73 (d, 1H), 7.54-6.64 (m, 13H), 5.64-4.99 (s, 1H), 5.27-2.00 (m, 12H), 3.52-3.06 (s, 3H), 3.51-3.45 (s, 3H), 2.44-2.03 (s, 3H), 1.11-0.41 (d, 3H).

HRMS-ESI (m/z) [M+H]⁺ calcd for C₃₉H₄₀N₅O₃: 626.3126, found 626.3133.

### Step C: N-(6-methoxy-3-pyridyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without the deprotection step, starting from Preparation Va as the appropriate acid (38 mg, 1.5 eq.) and the product of Step B (60 mg, 0.096 mmol) as the appropriate amine, the desired product was obtained (43 mg, 51%).

HRMS-ESI (m/z) [M+H]⁺ calcd for C₅₃H₅₇N₆O₆: 873.4334, found 873.4338.

### Example 192: 5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-N-(6-methoxy-3-pyridyl)-1,2-dimethyl-N-phenyl-pyrrole-3-carboxamide

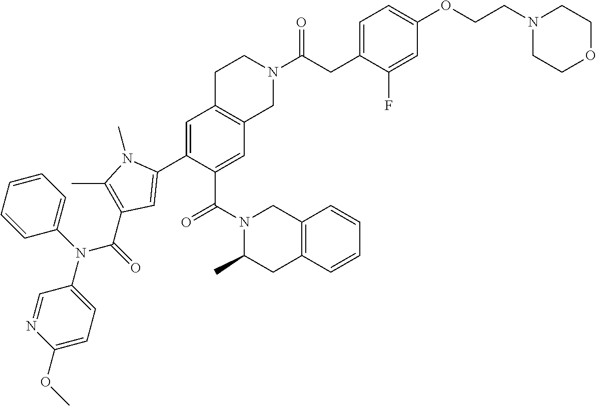 [SMILES: COc1ccc(N(C(=O)c2cc(-c3cc4c(cc3C(=O)N3Cc5ccccc5C[C@H]3C)CN(C(=O)Cc3ccc(OCCN5CCOCC5)cc3F)CC4)n(C)c2C)c2ccccc2)cn1]

Using General procedure 4c without the deprotection step, starting from Preparation Vb as the appropriate acid (41 mg, 1.5 eq.) and the product of Example 191, Step B (60 mg, 0.096 mmol) as the appropriate amine, the desired product was obtained (45 mg, 53%).

HRMS-ESI (m/z) [M+H]⁺ calcd for C₅₃H₅₆FN₆O₆: 891.4240, found 891.4240.

### Example 193: N-[1-(difluoromethyl)pyrazol-4-yl]-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

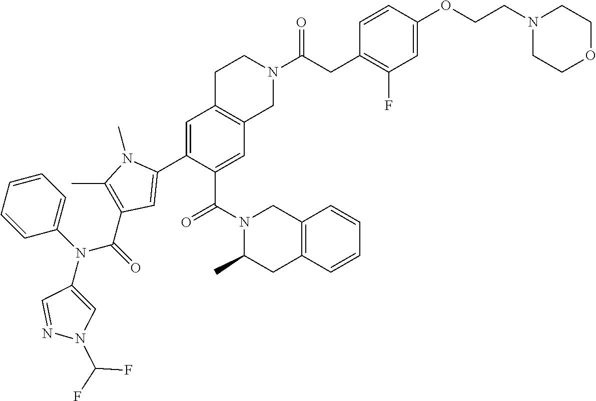 [SMILES: Cc1c(C(=O)N(c2ccccc2)c2cnn(C(F)F)c2)cc(-c2cc3c(cc2C(=O)N2Cc4ccccc4C[C@H]2C)CN(C(=O)Cc2ccc(OCCN4CCOCC4)cc2F)CC3)n1C]

Using General procedure 4c without the deprotection step, starting from Preparation Vb as the appropriate acid (37.5 mg, 1.2 eq.) and the product of Example 190, Step B (70 mg, 0.110 mmol) as the appropriate amine, the desired product was obtained (24 mg, 22%).

HRMS-ESI (m/z) [M+H]⁺ calcd for C₅₁H₅₃F₃N₇O₅: 900.4055, found 900.4058.

### Example 194: 5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-(1-methylpyrazolo[3,4-b]pyridin-5-yl)-N-phenyl-pyrrole-3-carboxamide

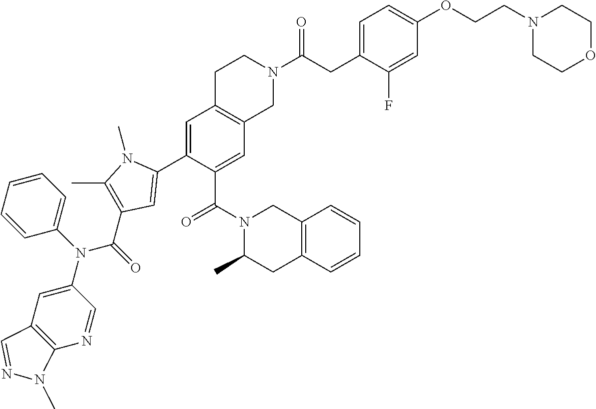 [SMILES: Cc1c(C(=O)N(c2ccccc2)c2cnc3c(cnn3C)c2)cc(-c2cc3c(cc2C(=O)N2Cc4ccccc4C[C@H]2C)CN(C(=O)Cc2ccc(OCCN4CCOCC4)cc2F)CC3)n1C]

### Step A: 1-methyl-N-phenyl-pyrazolo[3,4-b]pyridin-5-amine

Using General procedure 1b and 5-bromo-1-methyl-pyrazolo[3,4-b]pyridine (500 mg, 2.358 mmol) as the appropriate aryl bromide and aniline (242 mg, 1.1 eq.) afforded the title product (416 mg, 79%).

¹H NMR (500 MHz, DMSO-d6) δ ppm 8.39 (d, 1H), 8.22 (s, 1H), 7.98 (s, 1H), 7.89 (d, 1H), 7.22 (m, 2H), 7.00 (m, 2H), 6.79 (m, 1H), 4.03 (s, 3H); ¹³C NMR (125 MHz, DMSO-d6) δ ppm 147.0, 145.1, 144.7, 134.3, 131.2, 129.8, 119.7, 117.0, 115.6, 115.5, 34.2.

HRMS-ESI (m/z) [M+H]⁺ calcd for C₁₃H₁₃N₄: 225.1135, found 225.1135.

### Step B: 1,2-dimethyl-5-[7-[(3R)-3-methyl-3,4-dihydro-1H-isoquinoline-2-carbonyl]-1,2,3,4-tetrahydroisoquinolin-6-yl]-N-(1-methylpyrazolo[3,4-b]pyridin-5-yl)-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 8.41-6.74 (m, 14H), 5.6-5.02 (s, 1H), 5.13-2.07 (m, 11H), 4.06-3.08 (s, 6H), 2.43-2.09 (s, 3H), 1.05-0.36 (d, 3H).

HRMS-ESI (m/z) [M+H]⁺ calcd for C₄₀H₄₀N₇O₂: 650.3238, found 650.3237.

### Step C: 5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-(1-methylpyrazolo[3,4-b]pyridin-5-yl)-N-phenyl-pyrrole-3-carboxamide

Using General procedure 4c without the deprotection step, starting from Preparation Vb as the appropriate acid (59 mg, 1.5 eq.) and the product of Step B (90 mg, 0.139 mmol) as the appropriate amine, the desired product was obtained (84 mg, 66%).

HRMS-ESI (m/z) [M+H]⁺ calcd for C₅₄H₅₆FN₈O₅: 915.4352, found 915.4349.

### Example 195: 1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-(1-methylpyrazolo[3,4-b]pyridin-5-yl)-N-phenyl-pyrrole-3-carboxamide

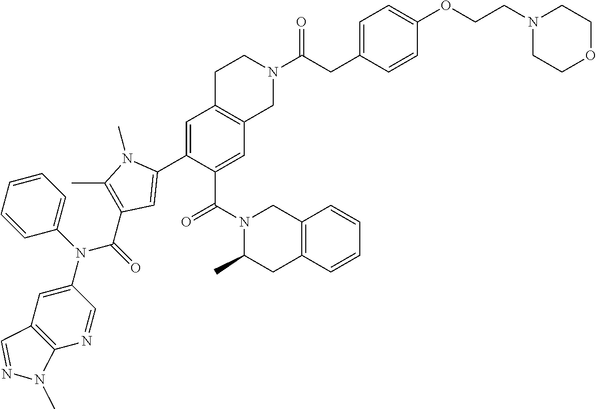 [SMILES: Cc1c(C(=O)N(c2ccccc2)c2cnc3c(cnn3C)c2)cc(-c2cc3c(cc2C(=O)N2Cc4ccccc4C[C@H]2C)CN(C(=O)Cc2ccc(OCCN4CCOCC4)cc2)CC3)n1C]

Using General procedure 4c without the deprotection step, starting from Preparation Va as the appropriate acid (55 mg, 1.5 eq.) and the product of Example 194, Step B (90 mg, 0.139 mmol) as the appropriate amine, the desired product was obtained (89 mg, 72%).

HRMS-ESI (m/z) [M+H]⁺ calcd for C₅₄H₅₇N₈O₅: 897.4446, found 897.4444.

### Example 196: N-[1-(2-methoxyethyl)pyrazol-4-yl]-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

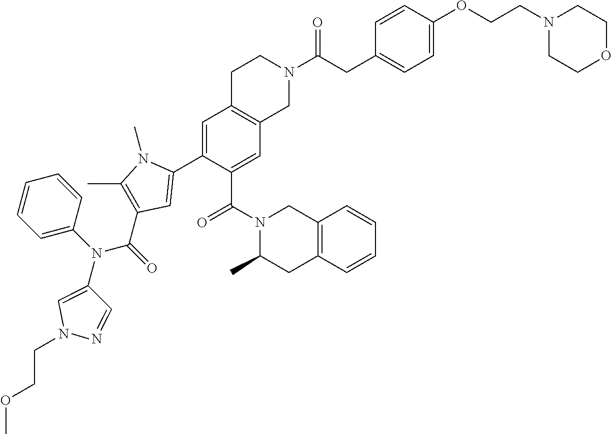 [SMILES: COCCn1cc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cn1]

### Step A: 1-(2-methoxyethyl)-N-phenyl-pyrazol-4-amine

Using General procedure 1b, starting from bromobenzene (445 mg, 2.83 mmol) as the appropriate aryl bromide and 1-(2-methoxyethyl)pyrazol-4-amine (400 mg, 1.0 eq.) as the appropriate amine, afforded the title product (139 mg, 23%).

LRMS (m/z) [M+H]⁺ calcd for C₁₂H₁₆N₃O: 218.1, found 218.0.

### Step B: N-[1-(2-methoxyethyl)pyrazol-4-yl]-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.73-6.83 (m, 13H), 5.49-5.00 (s, 1H), 4.96-2.35 (m, 15H), 3.34-3.07 (s, 6H), 2.41-2.06 (s, 3H), 1.03-0.47 (d, 3H).

HRMS-ESI: (m/z) [M+H]⁺ calcd for C₃₉H₄₃N₆O₃: 643.3391, found 643.3394.

### Step C. N-[1-(2-methoxyethyl)pyrazol-4-yl]-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without silyl deprotection step, starting from Preparation Va as the appropriate acid (44 mg, 1.5 eq.) and the product of Step B (70 mg, 0.11 mmol) as the appropriate amine, the desired product was obtained (37.0 mg, 38%).

HRMS-ESI (m/z) [M+H]⁺ calcd for C₅₃H₆₀N₇O₆:890.4599, found 890.4602.

### Example 197: 5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-(1-methylpyrazol-4-yl)-N-phenyl-pyrrole-3-carboxamide

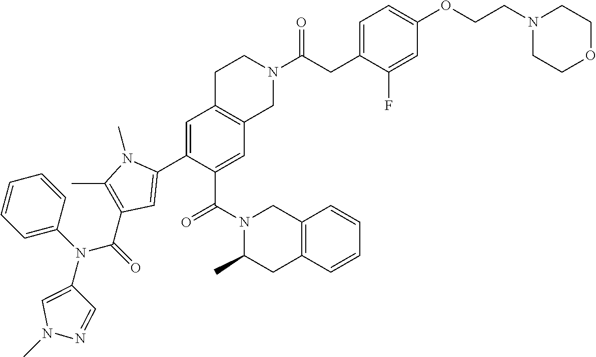 [SMILES: Cc1c(C(=O)N(c2ccccc2)c2cnn(C)c2)cc(-c2cc3c(cc2C(=O)N2Cc4ccccc4C[C@H]2C)CN(C(=O)Cc2ccc(OCCN4CCOCC4)cc2F)CC3)n1C]

Using General procedure 4c without silyl deprotection step, starting from Preparation Vb as the appropriate acid (46 mg, 1.5 eq.) and the product of Example 184, Step B (65 mg, 0.11 mmol) as the appropriate amine, the desired product was obtained (48.0 mg, 51%).

HRMS-ESI (m/z) [M+H]⁺ calcd for C₅₁H₅₅FN₇O₅:864.4243, found 864.4248.

### Example 198: N-(5-fluoro-6-methoxy-3-pyridyl)-1,2-dimethyl-5-[2-[2-[3-(6-methyl-2,6-diazaspiro[3.3]heptan-2-yl)phenyl]acetyl]-7-[(3R)-3-methyl-3,4-dihydro-1H-isoquinoline-2-carbonyl]-3,4-dihydro-1H-isoquinolin-6-yl]-N-phenyl-pyrrole-3-carboxamide

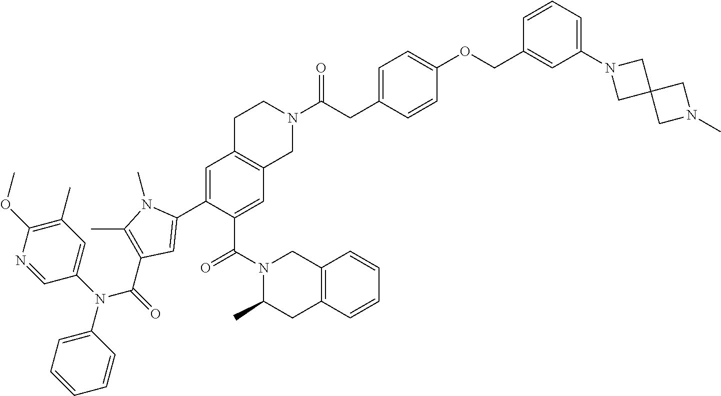 [SMILES: COc1ncc(N(C(=O)c2cc(-c3cc4c(cc3C(=O)N3Cc5ccccc5C[C@H]3C)CN(C(=O)Cc3ccc(OCc5cccc(N6CC7(CN(C)C7)C6)c5)cc3)CC4)n(C)c2C)c2ccccc2)cc1C]

### Step A: 2-[3-(6-methyl-2,6-diazaspiro[3.3]heptan-2-yl)phenyl]acetic acid

To a solution of methyl 2-(3-bromophenyl)acetate (0.60 g, 2.60 mmol) and 2-methyl-2,6-diazaspiro[3.3]heptane, 2,2,2-trifluoroacetic acid (1:2) (0.80 mL, 0.8 eq.) in 1,4-dioxane (5.0 mL) was added Cs₂CO₃ (1.20 g, 1.4 eq.) and [2-(2-aminoethyl)phenyl]-chloro-palladium, dicyclohexyl-[2-(2,4,6-triisopropylphenyl)phenyl]phosphane (1:1) (37 mg, 0.02 eq.), and the mixture was stirred until complete conversion was observed. After filtration, the crude product was hydrolysed using the General procedure 8, to give the desired product (279 mg, 43%).

¹H NMR (500 MHz, DMSO-d6) δ ppm 7.02 (t, 1H), 6.54 (d, 1H), 6.29 (brs., 1H), 6.22 (dd, 1H), 3.74 (s, 4H), 3.28 (s, 2H), 3.25 (s, 4H), 2.18 (s, 3H); ¹³C NMR (125 MHz, DMSO-d6) δ ppm 174.0, 151.9, 138.4, 128.7, 118.9, 112.9, 109.6, 66.1, 62.1, 45.9, 44.1, 34.4.

HRMS-ESI (m/z) [M+H]⁺ calcd for C₁₄H₁₉N₂O₂: 247.1441, found 247.1442.

### Step B: N-(5-fluoro-6-methoxy-3-pyridyl)-1,2-dimethyl-5-[2-[2-[3-(6-methyl-2,6-diazaspiro[3.3]heptan-2-yl)phenyl]acetyl]-7-[(3R)-3-methyl-3,4-dihydro-1H-isoquinoline-2-carbonyl]-3,4-dihydro-1H-isoquinolin-6-yl]-N-phenyl-pyrrole-3-carboxamide

Using General procedure 4c without silyl deprotection step, starting from the product of Step A (50 mg, 1.6 eq.) as the appropriate carboxylic acid and the product of Example 183, Step B (80 mg, 0.12 mmol) as the appropriate amine, afforded the title compound (35 mg, 32%).

HRMS-ESI (m/z) [M+H]⁺ calcd for C₅₃H₅₅FN₇O₄: 872.4294, found 872.4300.

### Example 199: N-[1-(difluoromethyl)pyrazol-4-yl]-5-[2-[2-[4-[2-(dimethylamino)ethoxy]phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

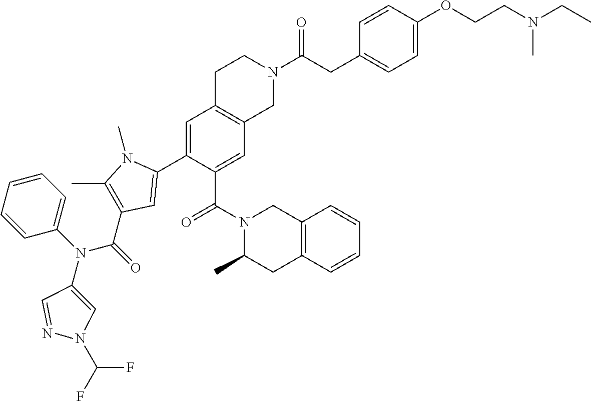 [SMILES: CCN(C)CCOc1ccc(CC(=O)N2CCc3cc(-c4cc(C(=O)N(c5ccccc5)c5cnn(C(F)F)c5)c(C)n4C)c(C(=O)N4Cc5ccccc5C[C@H]4C)cc3C2)cc1]

### Step A: 2-[4-[2-(dimethylamino)ethoxy]phenyl]acetic acid

Using the General procedure 7c and then General procedure 8, starting from methyl 2-(4-hydroxyphenyl)acetate (0.85 g, 5.11 mmol) as the appropriate phenol and 2-(dimethylamino)ethanol (0.68 g, 1.5 eq.) as the appropriate alcohol, the desired product was obtained (0.52 g, 53%).

¹H NMR (500 MHz, DMSO-d6) δ ppm 7.15 (d, 2H), 6.86 (d, 2H), 4.01 (t, 2H), 3.46 (s, 2H), 2.62 (t, 2H), 2.22 (s, 6H); ¹³C NMR (125 MHz, DMSO-d6) δ ppm 173.6, 157.6, 130.8, 127.7, 114.6, 66.1, 58.1, 45.9, 40.4.

HRMS-ESI (m/z) [M+H]⁺ calcd for C₁₂H₁₈NO₃: 224.1287, found 224.1283.

### Step B: N-[1-(difluoromethyl)pyrazol-4-yl]-5-[2-[2-[4-[2-(dimethylamino)ethoxy]phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

Using General procedure 4c without silyl deprotection step, starting from the product of Step A (30 mg, 1.2 eq.) as the appropriate carboxylic acid and the product of Example 190, Step B (70 mg, 0.11 mmol) as the appropriate amine, afforded the title compound (56 mg, 60%).

HRMS-ESI (m/z) [M+H]⁺ calcd for C₄₉H₅₂F₂N₇O₄: 840.4049, found 840.4042.

### Example 200: N-(4-fluorophenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-(1-methylpyrazol-4-yl)pyrrole-3-carboxamide

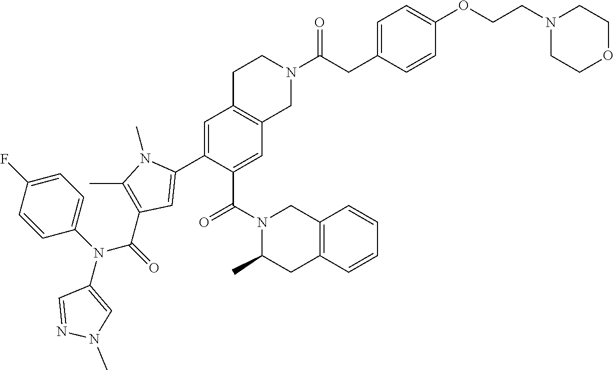 [SMILES: Cc1c(C(=O)N(c2ccc(F)cc2)c2cnn(C)c2)cc(-c2cc3c(cc2C(=O)N2Cc4ccccc4C[C@H]2C)CN(C(=O)Cc2ccc(OCCN4CCOCC4)cc2)CC3)n1C]

### Step A: N-(4-fluorophenyl)-1-methyl-pyrazol-4-amine

Using General procedure 1b, starting from 4-bromo-1-methyl-pyrazole (500 mg, 3.11 mmol) as the appropriate aryl bromide and 4-fluoroaniline (380 mg, 1.1 eq.) as the appropriate amine, afforded the title product (493 mg, 80%).

¹H NMR (500 MHz, DMSO-d6) δ ppm 7.61 (s, 1H), 7.44 (brs., 1H), 7.27 (s, 1H), 6.94 (m, 2H), 6.73 (m, 2H), 3.78 (s, 3H); ¹³C NMR (125 MHz, DMSO-d6) δ ppm 155.2, 143.9, 133.0, 125.7, 123.0, 115.9, 114.1, 39.2.

HRMS-ESI (m/z) [M+H]⁺ calcd for C₁₀H₁₁FN₃: 192.0933, found 192.0931.

### Step B: N-(4-fluorophenyl)-1,2-dimethyl-5-[7-[(3R)-3-methyl-3,4-dihydro-1H-isoquinoline-2-carbonyl]-1,2,3,4-tetrahydroisoquinolin-6-yl]-N-(1-methylpyrazol-4-yl)pyrrole-3-carboxamide

The title compound was obtained using the method described in Preparation VIIb and the product from Step A as the appropriate aniline for the amide formation.

¹H NMR (500 MHz, DMSO-d6) δ ppm 7.70-6.85 (m, 12H), 5.45-4.98 (s, 1H), 4.95-2.34 (m, 11H), 3.77-3.67 (s, 3H), 3.35-3.05 (s, 3H), 2.40-2.05 (s, 3H), 1.03-0.45 (d, 3H).

HRMS-ESI: (m/z) [M+H]⁺ calcd for C₃₇H₃₈FN₆O₂: 617.3040, found 617.3041.

### Step C. N-(4-fluorophenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-(1-methylpyrazol-4-yl)pyrrole-3-carboxamide

Using General procedure 4c without silyl deprotection step, starting from Preparation Va as the appropriate acid (52 mg, 1.5 eq.) and the product of Step B (80 mg, 0.13 mmol) as the appropriate amine, the desired product was obtained (83 mg, 74%).

HRMS-ESI (m/z) [M+H]⁺ calcd for C₅₁H₅₅FN₇O₅:864.4243, found 864.4242.

### Example 201: N-(5-cyano-6-methoxy-3-pyridyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

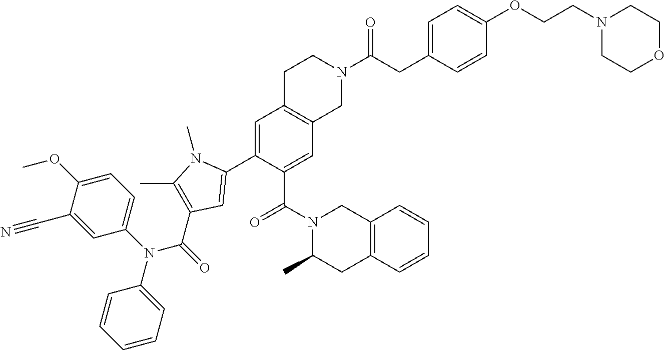 [SMILES: COc1ccc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cc1C#N]

### Step A: 5-anilino-2-methoxy-pyridine-3-carbonitrile

Using General procedure 1b, starting from 5-bromo-2-methoxy-pyridine-3-carbonitrile (770 mg, 3.61 mmol) as the appropriate aryl bromide and aniline (409 mg, 1.2 eq.) afforded the title product (710 mg, 87%).

¹H NMR (500 MHz, DMSO-d6) δ ppm 8.26 (s, 1H), 8.23 (d, 1H), 7.93 (d, 1H), 7.23 (m, 2H), 6.98 (m, 2H), 6.84 (m, 1H), 3.94 (s, 3H); ¹³C NMR (125 MHz, DMSO-d6) δ ppm 158.5, 143.8, 142.3, 134.7, 133.1, 129.9, 120.5, 116.2, 115.7, 95.6, 54.7.

HRMS-ESI (m/z) [M+H]⁺ calcd for C₁₃H₁₂N₃O: 226.0980, found 226.0972.

### Step B: N-(5-cyano-6-methoxy-3-pyridyl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 8.37-6.78 (m, 13H), 5.55/5.45/5.39/5.03 (s, 1H), 5.25-2.90 (m, 6H), 4.94/4.79/3.71 (br., 1H), 3.98/3.95/3.89 (s, 3H), 3.34/3.31/3.06 (s, 3H), 3.03-2.00 (m, 4H), 2.40/2.37/2.30/2.07 (s, 3H), 1.02/0.84/0.82/0.48 (d, 3H).

HRMS-ESI: (m/z) [M+H]⁺ calcd for C₄₀H₃₉N₆O₃: 651.3084, found 651.3081.

### Step C. N-(5-cyano-6-methoxy-3-pyridyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without silyl deprotection step, starting from Preparation Va as the appropriate acid (50 mg, 1.2 eq.) and the product of Step B (100 mg, 0.14 mmol) as the appropriate amine, the desired product was obtained (83 mg, 60%).

HRMS-ESI (m/z) [M+H]⁺ calcd for C₅₄H₅₆N₇O₆:898.4292, found 898.4287.

### Example 202: N-(5-cyano-6-methoxy-3-pyridyl)-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

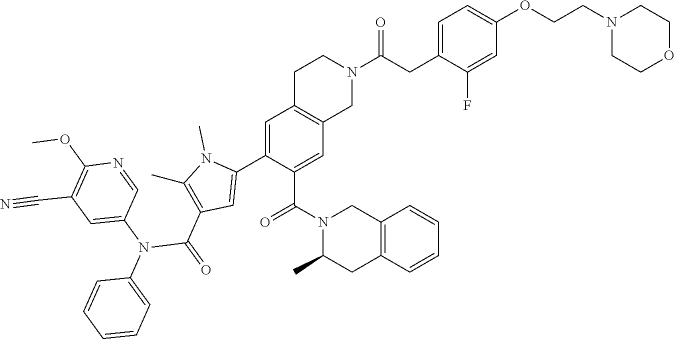 [SMILES: COc1ncc(N(C(=O)c2cc(-c3cc4c(cc3C(=O)N3Cc5ccccc5C[C@H]3C)CN(C(=O)Cc3ccc(OCCN5CCOCC5)cc3F)CC4)n(C)c2C)c2ccccc2)cc1C#N]

Using General procedure 4c without silyl deprotection step, starting from Preparation Vb as the appropriate acid (65 mg, 1.2 eq.) and the product of Example 201, Step B (120 mg, 0.18 mmol) as the appropriate amine, the desired product was obtained (121 mg, 72%).

HRMS-ESI (m/z) [M+H]⁺ calcd for C₅₄H₅₅FN₇O₆:916.4198, found 916.4189.

### Example 203: N-(3-cyano-4-methyl-phenyl)-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

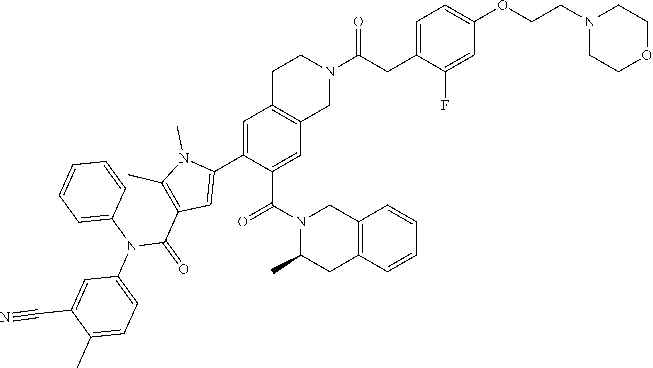 [SMILES: Cc1ccc(N(C(=O)c2cc(-c3cc4c(cc3C(=O)N3Cc5ccccc5C[C@H]3C)CN(C(=O)Cc3ccc(OCCN5CCOCC5)cc3F)CC4)n(C)c2C)c2ccccc2)cc1C#N]

### Step A: 5-anilino-2-methyl-benzonitrile

Using General procedure 1b, starting from bromobenzene (570 mg, 1.2 eq.) as the appropriate aryl bromide and 5-amino-2-methyl-benzonitrile (400 mg, 3.03 mmol) as the appropriate amine, afforded the title product (350 mg, 55%).

¹H NMR (500 MHz, DMSO-d6) δ ppm 8.38 (s, 1H), 7.29 (d, 1H), 7.28 (d, 1H), 7.27 (td, 2H), 7.27 (dd, 1H), 7.07 (dt, 2H), 6.89 (tt, 1H), 2.37 (s, 3H); ¹³C NMR (125 MHz, DMSO-d6) δ ppm 142.8, 142.6, 132.0, 131.8, 129.8, 121.6, 121.2, 119.0, 118.7, 117.9, 112.6, 19.4.

HRMS-ESI (m/z) [M+H]⁺ calcd for C₁₄H₁₃N₂: 209.1079, found 209.1074.

### Step B: N-(3-cyano-4-methyl-phenyl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.55-6.82 (m, 14H), 5.55-5.00 (s, 1H), 4.98-2.36 (m, 11H), 3.35-3.08 (s, 3H), 2.44-2.08 (s, 6H), 1.04-0.46 (d, 3H).

HRMS-ESI: (m/z) [M+H]⁺ calcd for C₄₁H₄₀N₅O₂: 634.3182, found 634.3179.

### Step C: N-(3-cyano-4-methyl-phenyl)-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

Using General procedure 4c without silyl deprotection step, starting from Preparation Vb as the appropriate acid (54 mg, 1.5 eq.) and the product of Step B (80 mg, 0.13 mmol) as the appropriate amine, the desired product was obtained (74 mg, 66%).

HRMS-ESI (m/z) [M+H]⁺ calcd for C₅₅H₅₆FN₆O₅: 899.4291, found 899.4296.

### Example 204: N-(3-cyano-4-methyl-phenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

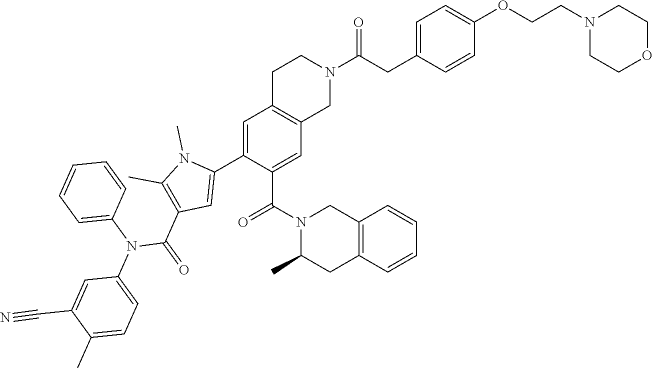 [SMILES: Cc1ccc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cc1C#N]

Using General procedure 4c without silyl deprotection step, starting from Preparation Va as the appropriate acid (57 mg, 1.5 eq.) and the product of Example 203, Step B (90 mg, 0.14 mmol) as the appropriate amine, the desired product was obtained (84 mg, 67%).

HRMS-ESI (m/z) [M+H]⁺ calcd for C₅₅H₅₇N₆O₅:881.4390, found 881.4386.

### Example 205: N-[1-(difluoromethyl)pyrazol-4-yl]-5-[7-[(3S)-3-(methoxymethyl)-3,4-dihydro-1H-isoquinoline-2-carbonyl]-2-[2-[4-(2-morpholinoethoxy)phenyl]acetyl]-3,4-dihydro-1H-isoquinolin-6-yl]-1,2-dimethyl-N-phenyl-pyrrole-3-carboxamide

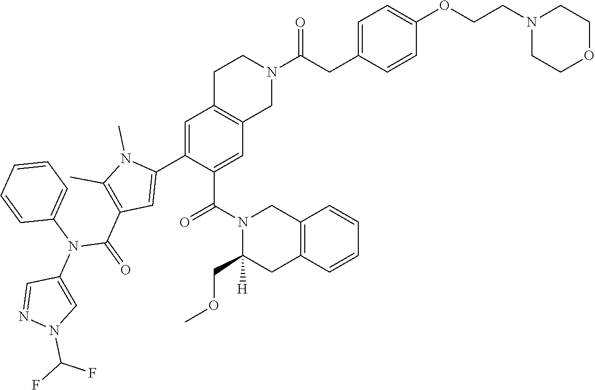 [SMILES: COC[C@@H]1Cc2ccccc2CN1C(=O)c1cc2c(cc1-c1cc(C(=O)N(c3ccccc3)c3cnn(C(F)F)c3)c(C)n1C)CCN(C(=O)Cc1ccc(OCCN3CCOCC3)cc1)C2]

### Step A: 02-benzyl 07-tert-butyl 6-[4-[[1-(difluoromethyl)pyrazol-4-yl]-phenyl-carbamoyl]-1,5-dimethyl-pyrrol-2-yl]-3,4-dihydro-1H-isoquinoline-2,7-dicarboxylate

Using General procedure 2b, starting from the product of Step B of Preparation IX (500 mg, 2.08 mmol) as the appropriate acid and the product of Example 190, Step A (249 mg, 1.2 eq.) as the appropriate aniline, the title compound was obtained (540 mg, 78%).

¹H NMR (500 MHz, DMSO-d6) δ ppm 8.18 (d, 1H), 7.89 (s, 1H), 7.72 (t, 1H), 7.42-7.27 (m, 10H), 7.39 (d, 1H), 6.80 (s, 1H), 5.13 (s, 2H), 5.00 (s, 1H), 4.66/4.60 (brs/brs, 2H), 3.63 (br, 2H), 3.12 (s, 3H), 2.78 (t, 2H), 2.45 (s, 3H), 1.26 (s, 9H); ¹³C NMR (125 MHz, DMSO-d6) δ ppm 132.9, 130.0, 128.1, 122.5, 110.9, 108.3, 66.8, 45.5, 41.1, 31.5, 28.4, 27.8, 12.2.

HRMS-ESI (m/z) [M+H]⁺ calcd for C₃₉H₄₀F₂N₅O₅: 696.2997, found 696.2995.

### Step B: tert-butyl 6-[4-[[1-(difluoromethyl)pyrazol-4-yl]-phenyl-carbamoyl]-1,5-dimethyl-pyrrol-2-yl]-1,2,3,4-tetrahydroisoquinoline-7-carboxylate

According to General procedure 6b, the product from Step A (530 mg, 0.76 mmol) in MeOH (8 mL) was hydrogenated using 16 mg of Pd catalyst (10% on charcoal) affording the title product (415 mg, 97%).

¹H NMR (500 MHz, DMSO-d6) δ ppm 8.18 (d, 1H), 7.73 (t, 1H), 7.58 (d, 1H), 7.42 (s, 1H), 7.40 (t, 2H), 7.32 (t, 1H), 7.29 (d, 2H), 6.68 (s, 1H), 4.97 (s, 1H), 3.85 (s, 2H), 3.11 (s, 3H), 2.92 (t, 2H), 2.64 (t, 2H), 2.45 (s, 3H), 1.25 (s, 9H)); ¹³C NMR (125 MHz, DMSO-d6) δ ppm 138.1, 133.3, 130.0, 128.7, 128.0, 127.8, 122.5, 110.9, 108.1, 47.7, 43.2, 31.4, 28.9, 27.8, 12.2.

HRMS-ESI (m/z) [M+H]⁺ calcd for C₃₁H₃₄F₂N₅O₃: 562.2630, found 562.2626.

### Step C. 6-[4-[[1-(difluoromethyl)pyrazol-4-yl]-phenyl-carbamoyl]-1,5-dimethyl-pyrrol-2-yl]-2-[2-[4-(2-morpholinoethoxy)phenyl]acetyl]-3,4-dihydro-1H-isoquinoline-7-carboxylic acid

Using General procedure 4c without silyl deprotection step, starting from Preparation Va as the appropriate acid (230 mg, 1.2 eq.) and the product from Step B (405 mg, 0.72 mmol) as the appropriate amine afforded the title compound after deprotection using General Procedure 3a (287 mg, 63%).

¹H NMR (500 MHz, DMSO-d6) δ ppm 12.61 (brs, 1H), 8.17 (s, 1H), 7.73 (t, 1H), 7.62/7.61 (s/s, 1H), 7.60 (s, 1H), 7.40 (t, 2H), 7.32 (t, 1H), 7.27 (d, 2H), 7.16/7.15 (d/d, 2H), 6.88/6.84 (d/d, 2H), 6.71 (s, 1H), 4.99/4.98 (s/s, 1H), 4.74/4.65 (s/s, 2H), 4.06/4.03 (t/t, 2H), 3.72/3.71 (s/s, 2H), 3.71/3.68 (t/t, 2H), 3.57 (t, 4H), 3.13/3.12 (s/s, 3H), 2.72/2.67 (t/t, 2H), 2.68/2.66 (t/t, 2H), 2.46 (brt, 4H), 2.42/2.41 (s/s, 3H); ¹³C NMR (125 MHz, DMSO-d6) δ ppm 138.2, 132.9/132.8, 130.5/130.5, 129.9, 128.6, 128.4/128.3, 127.6, 122.7/122.6, 114.9/114.7, 110.9, 108.5, 66.6, 65.7/65.6, 57.5, 54.1, 47/43.8, 43/39.2, 39.4, 31.5, 29/28.2, 12.0.

HRMS-ESI (m/z) [M+H]⁺ calcd for C₄₁H₄₃F₂N₆O₆: 753.3212, found 753.3211.

### Step D: N-[1-(difluoromethyl)pyrazol-4-yl]-5-[7-[(3S)-3-(methoxymethyl)-3,4-dihydro-1H-isoquinoline-2-carbonyl]-2-[2-[4-(2-morpholinoethoxy)phenyl]acetyl]-3,4-dihydro-1H-isoquinolin-6-yl]-1,2-dimethyl-N-phenyl-pyrrole-3-carboxamide

Using General Procedure 4c without silyl deprotection step, starting from the product of Step C (90 mg, 0.12 mmol) as the appropriate acid and the compound of Example 52, Step C (25 mg, 1.2 eq.) as the appropriate amine, afforded the title product (65 mg, 60%).

HRMS-ESI (m/z) [M+H]⁺ calcd for C₅₂H₅₆F₂N₇O₆: 912.4260, found 912.4256.

### Example 206: 5-[7-[(3S)-3-(2-azaspiro[3.3]heptan-2-ylmethyl)-3,4-dihydro-1H-isoquinoline-2-carbonyl]-2-[2-[4-(2-morpholinoethoxy)phenyl]acetyl]-3,4-dihydro-1H-isoquinolin-6-yl]-N-[1-(difluoromethyl)pyrazol-4-yl]-1,2-dimethyl-N-phenyl-pyrrole-3-carboxamide

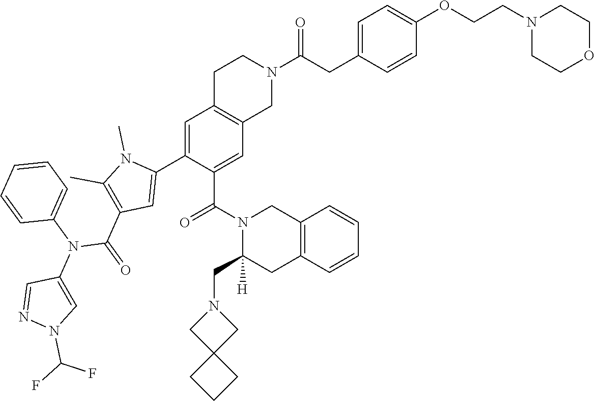 [SMILES: Cc1c(C(=O)N(c2ccccc2)c2cnn(C(F)F)c2)cc(-c2cc3c(cc2C(=O)N2Cc4ccccc4C[C@H]2CN2CC4(CCC4)C2)CN(C(=O)Cc2ccc(OCCN4CCOCC4)cc2)CC3)n1C]

Using General Procedure 4c without silyl deprotection step, starting from the product of Example 205, Step C (90 mg, 0.12 mmol) as the appropriate acid and the compound of Example 180, Step G (25 mg, 1.2 eq.) as the appropriate amine, afforded the title product (65 mg, 60%).

HRMS-ESI (m/z) [M+H]⁺ calcd for C₅₇H₆₃F₂N₈O₅: 977.4889, found 977.4887.

### Example 207: N-[l-[2-(dimethylamino)ethyl]pyrazol-4-yl]-1,2-dimethyl-5-[7-[(3R)-3-methyl-3,4-dihydro-1H-isoquinoline-2-carbonyl]-2-[2-[4-(2-tetrahydropyran-4-ylethoxy)phenyl]acetyl]-3,4-dihydro-1H-isoquinolin-6-yl]-N-phenyl-pyrrole-3-carboxamide

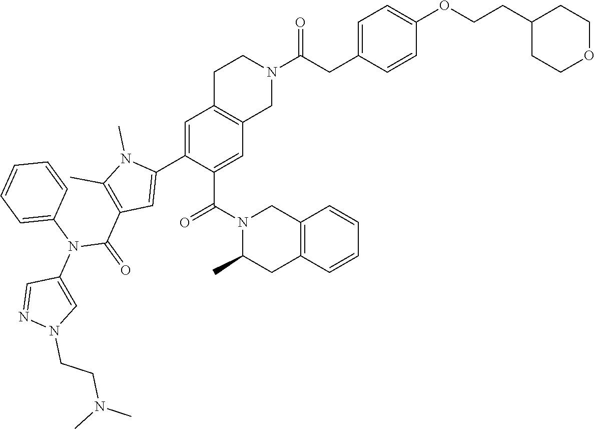 [SMILES: Cc1c(C(=O)N(c2ccccc2)c2cnn(CCN(C)C)c2)cc(-c2cc3c(cc2C(=O)N2Cc4ccccc4C[C@H]2C)CN(C(=O)Cc2ccc(OCCC4CCOCC4)cc2)CC3)n1C]

### Step A: 2-(4-bromopyrazol-1-yl)-N,N-dimethyl-ethanamine

To 4-bromo-1H-pyrazole (1.0 g, 6.8 mmol) and 2-chloro-N,N-dimethyl-ethanamine, hydrogen chloride (1:1) (1.47 g, 1.5 eq.) in DMF (5 mL) was added K₂CO₃ (2.35 g, 2.5 eq.) and the mixture was stirred at rt until complete conversion was observed. The reaction mixture was poured into water, extracted with DCM, dried over MgSO₄, filtered and concentrated, then purified by flash chromatography using DCM and MeOH as eluents, to give the title compound (0.57 g, 39%).

¹H NMR (500 MHz, DMSO-d6) δ ppm 7.96 (d, 1H), 7.51 (d, 1H), 4.17 (t, 2H), 2.60 (t, 2H), 2.14 (s, 6H); ¹³C NMR (125 MHz, DMSO-d6) δ ppm 139.1, 130.9, 91.6, 58.8, 50.4, 45.5.

HRMS-ESI (m/z) [M+H]⁺ calcd for C₇H₁₃N₃Br: 218.0293, found 218.0287.

### Step B: 1-[2-(dimethylamino)ethyl]-N-phenyl-pyrazol-4-amine

Using General procedure 1b, starting from the product from Step A (558 mg, 2.56 mmol) as the appropriate aryl bromide and aniline (286 mg, 1.2 eq.) as the appropriate amine, afforded the title product (424 mg, 72%).

¹H NMR (500 MHz, DMSO-d6) δ ppm 7.67 (d, 1H), 7.49 (s, 1H), 7.29 (d, 1H), 7.11 (t, 2H), 6.75 (d, 2H), 6.60 (t, 1H), 4.13 (t, 2H), 2.63 (t, 2H), 2.16 (s, 6H); ¹³C NMR (125 MHz, DMSO-d6) δ ppm 147.3, 132.9, 129.5, 125.1, 122.5, 117.3, 113.2, 59.2, 50.2, 45.6.

HRMS-ESI (m/z) [M+H]⁺ calcd for C₁₃H₁₉N₄: 231.1610, found 231.1605.

### Step C. N-[1-[2-(dimethylamino)ethyl]pyrazol-4-yl]-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step B as the appropriate aniline for the amide formation.

¹H NMR (500 MHz, DMSO-d6) δ ppm 7.75-6.81 (m, 13H), 5.46-4.96 (s, 1H), 4.95-2.04 (m, 15H), 3.33-3.05 (s, 3H), 2.41-2.06 (s, 9H), 1.03-0.47 (d, 3H).

HRMS-ESI: (m/z) [M+H]⁺ calcd for C₄₀H₄₆N₇O₂: 656.3713, found 656.3708.

### Step D: N-[1-[2-(dimethylamino)ethyl]pyrazol-4-yl]-1,2-dimethyl-5-[7-[(3R)-3-methyl-3,4-dihydro-1H-isoquinoline-2-carbonyl-2-[2-[4-(2-tetrahydropyran-4-ylethoxy)phenylacetyl]-3,4-dihydro-1H-isoquinolin-6-yl]-N-phenyl-pyrrole-3-carboxamide

Using General procedure 4c without silyl deprotection step, starting from Preparation Vc as the appropriate acid (35 mg, 1.5 eq.) and the product of Step C (58 mg, 0.09 mmol) as the appropriate amine, the desired product was obtained (58 mg, 73%).

HRMS-ESI (m/z) [M+H]⁺ calcd for C₅₅H₆₄N₇O₅:902.4969, found 902.4961.

### Example 208: 5-[2-[2-[4-[2-(dimethylamino)ethoxy]phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-(1-methylpyrazol-4-yl)-N-phenyl-pyrrole-3-carboxamide

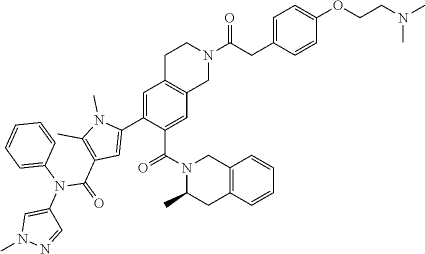

Using General procedure 4c without silyl deprotection step, starting from the product of Example 199, Step A (36 mg, 1.5 eq.) as the appropriate carboxylic acid and the product of Example 184, Step B (65 mg, 0.11 mmol) as the appropriate amine, afforded the title compound (37 mg, 43%).

HRMS-ESI (m/z) [M+H]⁺ calcd for C₄₉H₅₄N₇O₄: 804.4237, found 804.4234.

### Example 209: N-(3-cyano-4-methoxy-phenyl)-N-(4-fluorophenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

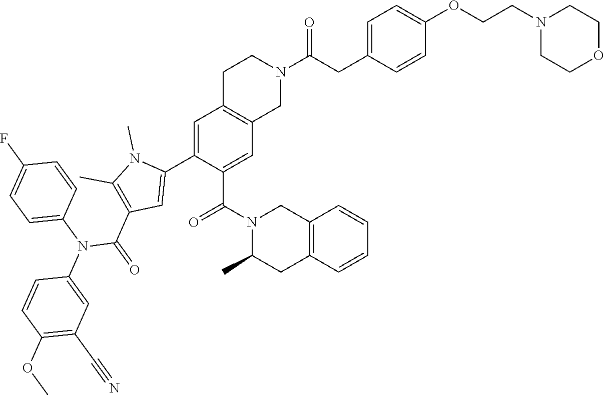 [SMILES: COc1ccc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(F)cc2)cc1C#N]

### Step A: 5-(4-fluoroanilino)-2-methoxy-benzonitrile

Using General procedure 1b, starting from 5-bromo-2-methoxy-benzonitrile (500 mg, 1.0 eq.) as the appropriate aryl bromide and 4-fluoroaniline (262 mg, 2.36 mmol) as the appropriate amine, afforded the title product (347 mg, 61%).

LRMS (m/z) [M+H]⁺ calcd for C₁₄H₁₂FN₂O: 243.09, found 243.0.

### Step B: N-(3-cyano-4-methoxy-phenyl)-N-(4-fluorophenyl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

The title compound was obtained using the method described in Preparation VIIb and the product from Step A as the appropriate aniline for the amide formation.

LRMS (m/z) [M+H]⁺ calcd for C₅₆H₄₉FN₅O₅: 890.37, found 890.0.

### Step C: N-(3-cyano-4-methoxy-phenyl)-N-(4-fluorophenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]pyrrole-3-carboxamide

Using General procedure 4c without silyl deprotection step, starting from Preparation Va as the appropriate acid (52 mg, 1.5 eq.) and the product of Step B (88 mg, 0.13 mmol) as the appropriate amine, the desired product was obtained (76 mg, 63%).

HRMS-ESI (m/z) [M+H]⁺ calcd for C₅₅H₅₆FN₆O₆:915.4245, found 915.4243.

### Example 210: N-(1-ethyl-3-methyl-pyrazol-4-yl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

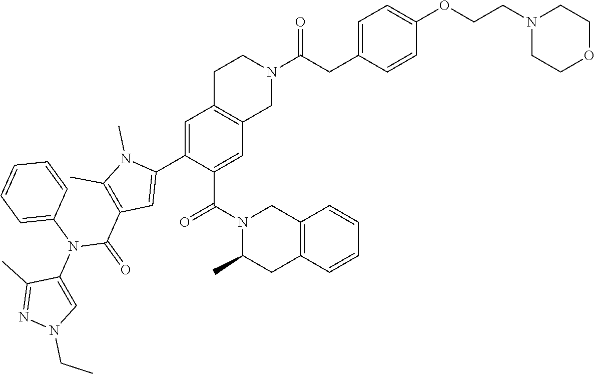 [SMILES: CCn1cc(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)c(C)n1]

### Step A: 1-ethyl-3-methyl-N-phenyl-pyrazol-4-amine

Using General procedure 1b, starting from 4-bromo-1-ethyl-3-methyl-pyrazole (400 mg, 1.0 eq.) as the appropriate aryl bromide and aniline (197 mg, 2.11 mmol) afforded the title product (330 mg, 77%).

LRMS (m/z) [M+H]⁺ calcd for C₁₂H₁₆N₃: 202.1, found 202.0.

### Step B: N-(1-ethyl-3-methyl-pyrazol-4-yl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.66-6.85 (m, 12H), 5.62-5.20 (s, 1H), 4.98-2.35 (m, 13H), 3.35-3.06 (s, 3H), 2.40-1.65 (s, 6H), 1.33-1.22 (t, 3H), 1.04-0.46 (d, 3H).

HRMS-ESI (m/z) [M+H]⁺ calcd for C₃₉H₄₃N₆O₂: 627.3447, found 627.3441.

### Step C. N-(1-ethyl-3-methyl-pyrazol-4-yl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without silyl deprotection step, starting from Preparation Va as the appropriate acid (46 mg, 1.5 eq.) and the product of Step B (72 mg, 0.11 mmol) as the appropriate amine, the desired product was obtained (77 mg, 76%).

HRMS-ESI (m/z) [M+H]⁺ calcd for C₅₃H₆₀N₇O₅:874.4656, found 874.4649.

### Example 211: N-[1-(difluoromethyl)pyrazol-4-yl]-5-[7-[(3S)-3-(fluoromethyl)-3,4-dihydro-1H-isoquinoline-2-carbonyl]-2-[2-[4-(2-morpholinoethoxy)phenyl]acetyl]-3,4-dihydro-1H-isoquinolin-6-yl]-1,2-dimethyl-N-phenyl-pyrrole-3-carboxamide

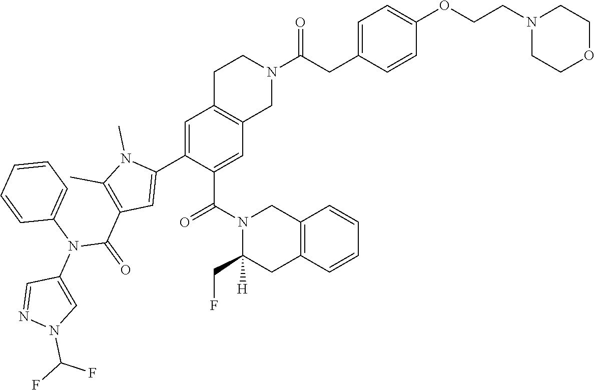 [SMILES: Cc1c(C(=O)N(c2ccccc2)c2cnn(C(F)F)c2)cc(-c2cc3c(cc2C(=O)N2Cc4ccccc4C[C@H]2CF)CN(C(=O)Cc2ccc(OCCN4CCOCC4)cc2)CC3)n1C]

### Step A: (3S)-3-(fluoromethyl)-2-(p-tolylsulfonyl)-3,4-dihydro-1H-isoquinoline

To the product of Example 169, Step A (4.2 g, 13.2 mmol) in DCM (120 mL) was added DAST (3.5 mL, 2.0 eq.) at −78° C. After 2 hours, the reaction mixture was warmed to rt and stirred until complete conversion was observed. The reaction mixture was quenched with sat. NaHCO₃ was extracted with DCM and the organic phase was washed with brine, dried, and concentrated filtered and concentrated, then purified by flash chromatography using Heptane and EtOAc as eluents, to give the title compound (1.3 g, 30%).

¹H NMR (500 MHz, DMSO-d6) δ ppm 7.65 (m, 2H), 7.28 (m, 2H), 7.16-7.02 (m, 4H), 4.52/4.30 (d+d, 2H), 4.48-4.22 (m, 2H), 4.34 (m, 1H), 2.78/2.70 (m+m, 2H), 2.32 (s, 3H); ¹³C NMR (125 MHz, DMSO-d6) δ ppm 130.1, 127.5, 83.4, 51.9, 44.4, 28.5, 21.4.

HRMS-ESI (m/z) [M+H]⁺ calcd for C₁₇H₁₉FNO₂S: 320.1120, found 320.1115.

### Step B: (3S)-3-(fluoromethyl)-1,2,3,4-tetrahydroisoquinoline

To a solution of the product from Step A (1.3 g, 4.05 mmol) in MeOH (55 mL) was added Mg powder (0.89 g, 9.0 eq.) and the reaction mixture was stirred until complete deprotection. After filtration through a pad of Celite and concentration the title compound was obtained (0.52 g, 78%).

¹H NMR (500 MHz, DMSO-d6) δ ppm 7.13-7.00 (m, 4H), 4.47/4.38 (ddd+ddd, 2H), 3.91/3.88 (d+d, 2H), 3.09 (m, 1H), 2.67/2.50 (dd+dd, 2H), 2.51 (brs, 1H); ¹³C NMR (125 MHz, DMSO-d6) δ ppm 86.8, 53.2, 47.7, 30.1.

HRMS-ESI (m/z) [M+H]⁺ calcd for C₁₀H₁₃FN: 166.1032, found 166.1026.

### Step C. N-[1-(difluoromethyl)pyrazol-4-yl]-5-[7-[(3S)-3-(fluoromethyl)-3,4-dihydro-1H-isoquinoline-2-carbonyl]-2-[2-[4-(2-morpholinoethoxy)phenyl]acetyl]-3,4-dihydro-1H-isoquinolin-6-yl]-1,2-dimethyl-N-phenyl-pyrrole-3-carboxamide

Using General Procedure 4c without silyl deprotection step, starting from the product of Example 205, Step C (90 mg, 0.12 mmol) as the appropriate acid and the compound from Step B (24 mg, 1.2 eq.) as the appropriate amine, afforded the title product (56 mg, 52%).

HRMS-ESI (m/z) [M+H]⁺ calcd for C₅₁H₅₃F₃N₇O₅: 900.4060, found 900.4059.

### Example 212: N-[1-(difluoromethyl)pyrazol-4-yl]-1,2-dimethyl-5-[7-[(3R)-3-methyl-3,4-dihydro-1H-isoquinoline-2-carbonyl]-2-[2-[4-(3-morpholinoprop-1-ynyl)phenyl]acetyl]-3,4-dihydro-1H-isoquinolin-6-yl]-N-phenyl-pyrrole-3-carboxamide

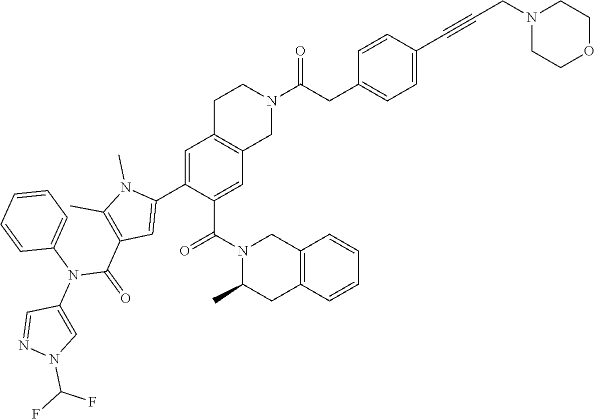 [SMILES: Cc1c(C(=O)N(c2ccccc2)c2cnn(C(F)F)c2)cc(-c2cc3c(cc2C(=O)N2Cc4ccccc4C[C@H]2C)CN(C(=O)Cc2ccc(C#CCN4CCOCC4)cc2)CC3)n1C]

### Step A: 2-[4-(3-morpholinoprop-1-ynyl)phenyl]acetic acid

To the solution of 3-bromoprop-1-yne (0.93 g, 3 eq.) and morpholine (0.5 g, 2.2 eq.) in 1,4-dioxane (5 mL) was added K₂CO₃ (2.00 g, 5.5 eq.) and the mixture was stirred at rt until complete conversion was observed. The reaction mixture was filtered and concentrated. To the residue, methyl 2-(4-bromophenyl)acetate (0.60 g, 2.62 mmol) in 1,4-dioxane (5.0 mL), iodocopper (0.10 g, 0.8 eq), Cs₂CO₃ (1.20 g, 1.4 eq.) and [2-(2-aminoethyl)phenyl]-chloro-palladium, dicyclohexyl-[2-(2,4,6-triisopropylphenyl)phenyl]phosphane (1:1) (37 mg, 0.02 eq.) were added, and the mixture was stirred at 120° C. until complete conversion was observed. After filtration the crude product was hydrolysed using the General procedure 8 to afford the desired product (279 mg, 41%).

¹H NMR (500 MHz, DMSO-d6) δ ppm 12.38 (brs, 1H), 7.40 (d, 2H), 7.27 (d, 2H), 3.65 (br, 4H), 3.60 (s, 2H), 2.61 (br, 6H); ¹³C NMR (125 MHz, DMSO-d6) δ ppm 131.8, 130.3, 66.2, 52.1, 40.9.

HRMS-ESI (m/z) [M+H]⁺ calcd for C₁₅H₁₈NO₃: 260.1287, found 260.1282.

### Step B: N-[1-(difluoromethyl)pyrazol-4-yl]-1,2-dimethyl-5-[7-[(3R)-3-methyl-3,4-dihydro-1H-isoquinoline-2-carbonyl]-2-[2-[4-(3-morpholinoprop-1-ynyl)phenyl]acetyl]-3,4-dihydro-1H-isoquinolin-6-yl]-N-phenyl-pyrrole-3-carboxamide

Using General procedure 4c without silyl deprotection step, starting from the product of Step A (44 mg, 1.2 eq.) as the appropriate carboxylic acid and the product of Example 190, Step B (90 mg, 0.14 mmol) as the appropriate amine, afforded the title compound (18 mg, 14%).

HRMS-ESI (m/z) [M+H]⁺ calcd for C₅₂H₅₂F₂N₇O₄: 876.4049, found 876.4041.

### Example 213: N-(5-cyano-1,2-dimethyl-pyrrol-3-yl)-5-[2-[2-[4-[2-(dimethylamino)ethoxy]-2-fluoro-phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

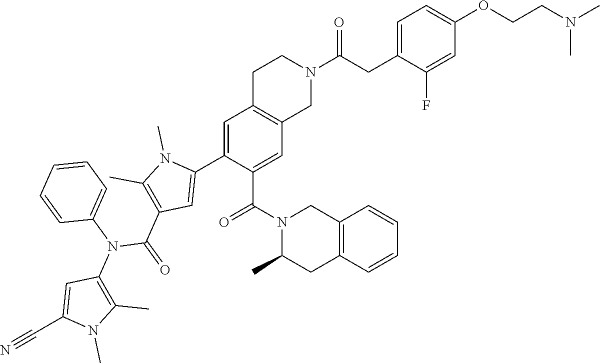 [SMILES: Cc1c(N(C(=O)c2cc(-c3cc4c(cc3C(=O)N3Cc5ccccc5C[C@H]3C)CN(C(=O)Cc3ccc(OCCN(C)C)cc3F)CC4)n(C)c2C)c2ccccc2)cc(C#N)n1C]

### Step A: 2-[4-[2-(dimethylamino)ethoxy]-2-fluoro-phenyl]acetic acid

Using the General procedure 7c and then General procedure 8, starting from methyl 2-(2-fluoro-4-hydroxy-phenyl)acetate (0.85 g, 4.61 mmol) as the appropriate phenol and 2-(dimethylamino)ethanol (0.62 g, 1.5 eq.) as the appropriate alcohol, the desired product was obtained (0.6 g, 54%).

¹H NMR (500 MHz, DMSO-d6) δ ppm 10.89 (brs, 1H), 7.25 (t, 1H), 6.89 (dd, 1H), 6.80 (dd, 1H), 4.35 (t, 2H), 3.54 (s, 2H), 3.36 (br, 2H), 2.73 (s, 6H); ¹³C NMR (125 MHz, DMSO-d6) δ ppm 132.8, 111.1, 102.6, 63.6, 55.6, 43.3, 34.1.

HRMS-ESI (m/z) [M+H]⁺ calcd for C₁₂H₁₇FNO₃: 242.1192, found 242.1187.

### Step B: N-(5-cyano-1,2-dimethyl-pyrrol-3-yl)-5-[2-[2-[4-[2-(dimethylamino)ethoxy]-2-fluoro-phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

Using General procedure 4c without silyl deprotection step, starting from the product of Step A (87 mg, 1.8 eq.) as the appropriate carboxylic acid and the product of Step D of Example 3 (94 mg, 0.14 mmol) as the appropriate amine, afforded the title compound (18 mg, 15%).

HRMS-ESI (m/z) [M+H]⁺ calcd for C₅₂H₅₅FN₇O₄: 860.4299, found 860.4294.

### Example 214: N-[5-cyano-1-(difluoromethyl)-2-methyl-pyrrol-3-yl]-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

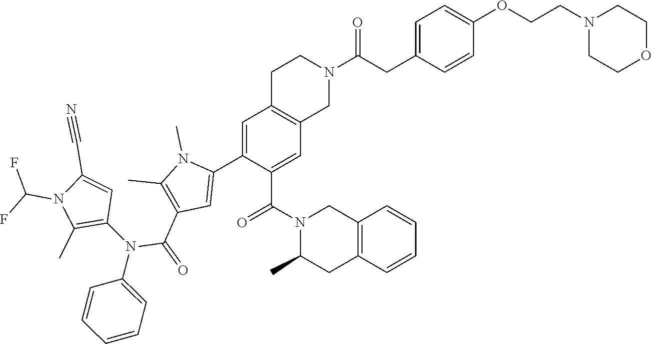 [SMILES: Cc1c(C(=O)N(c2ccccc2)c2cc(C#N)n(C(F)F)c2C)cc(-c2cc3c(cc2C(=O)N2Cc4ccccc4C[C@H]2C)CN(C(=O)Cc2ccc(OCCN4CCOCC4)cc2)CC3)n1C]

### Step A: 4-anilino-1-(difluoromethyl)-5-methyl-pyrrole-2-carbonitrile

Using General procedure 1b, starting from 4-bromo-]-(difluoromethyl)-5-methyl-pyrrole-2-carbonitrile (1.0 g, 4.25 mmol) as the appropriate aryl bromide and aniline (0.54 g, 1.5 eq.) as the appropriate amine, afforded the title product (0.67 g, 63%).

¹H NMR (400 MHz, DMSO-d6) δ ppm 7.81 (t, 1H), 7.39 (s, 1H), 7.22 (s, 1H), 7.11 (m, 2H), 6.65 (m, 1H), 6.63 (m, 2H), 2.25 (s, 3H); ¹³C NMR (100 MHz, DMSO-d6) δ ppm 147.1, 129.6, 129.5, 126.0, 121.9, 118.0, 113.6, 112.9, 109.7, 97.9, 9.8.

HRMS-ESI (m/z) [M+H]⁺ calcd for C₁₃H₁₂F₂N₃: 248.0999, found 248.0992.

### Step B: N-[5-cyano-1-(difluoromethyl)-2-methyl-pyrrol-3-yl]-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.84/7.80 (t/t, 1H), 7.33-6.86 (m, 12H), 5.57-5.09 (s, 1H), 5.27-2.03 (m, 11H), 3.36-3.05 (s, 3H), 2.41-1.97 (s, 6H), 1.05-0.48 (d, 3H).

HRMS-ESI (m/z) [M+H]⁺ calcd for C₄₀H₃₉F₂N₆O₂: 673.3103, found 673.3102.

### Step C. N-[5-cyano-1-(difluoromethyl)-2-methyl-pyrrol-3-yl-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without silyl deprotection step, starting from Preparation Va as the appropriate acid (37 mg, 1.2 eq.) and the product of Step B (70 mg, 0.10 mmol) as the appropriate amine, the desired product was obtained (71 mg, 74%).

HRMS-ESI (m/z) [M+H]⁺ calcd for C₅₄H₅₆F₂N₇O₅:920.4311, found 920.4309.

### Example 215: N-[1-(difluoromethyl)-5-methyl-pyrazol-4-yl]-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

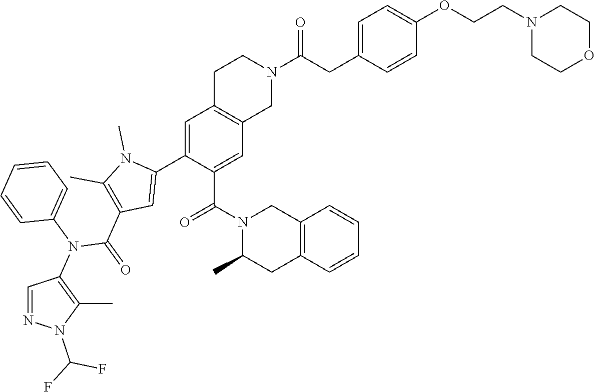 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cnn1C(F)F]

### Step A: 1-(difluoromethyl)-5-methyl-N-phenyl-pyrazol-4-amine

Using General procedure 1b, starting from 4-bromo-]-(difluoromethyl)-5-methyl-pyrazole (530 mg, 2.50 mmol) as the appropriate aryl bromide and aniline (280 mg, 1.2 eq.), afforded the title product (427 mg, 77%).

¹H NMR (500 MHz, DMSO-d6) δ ppm 7.75 (t, 1H), 7.67 (s, 1H), 7.31 (s, 1H), 7.11 (td, 2H), 6.64 (tt, 1H), 6.63 (dt, 2H), 2.26 (s, 3H); ¹³C NMR (125 MHz, DMSO-d6) δ ppm 147.4, 139.3, 132.8, 129.6, 125.1, 117.8, 113.3, 112.2, 8.8.

HRMS-ESI (m/z) [M+H]⁺ calcd for C₃₈H₃₉F₂N₆O₂: 224.0994, found 224.0995.

### Step B: N-[1-(difluoromethyl)-5-methyl-pyrazol-4-yl]-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.72 (t, 1H), 7.64-6.82 (m, 12H), 5.62-5.05 (s, 1H), 5.27-2.00 (m, 11H), 3.40-3.01 (s, 3H), 2.85 (brs, 1H), 2.44-1.92 (s, 6H), 1.07-0.42 (d, 3H)

HRMS-ESI: (m/z) [M+H]⁺ calcd for C₃₈H₃₉F₂N₆O₂: 649.3097, found 649.3101.

### Step C: N-[1-(difluoromethyl)-5-methyl-pyrazol-4-yl]-1,2-dimethyl-5-[7-[(3R)-3-methyl-3,4-dihydro-1H-isoquinoline-2-carbonyl-2-[2-[4-(2-morpholinoethoxy)phenyl]acetyl]-3,4-dihydro-1H-isoquinolin-6-yl]-N-phenyl-pyrrole-3-carboxamide

Using General procedure 4c without deprotection, starting from Preparation Va as the appropriate acid (44 mg, 1.5 eq.) and the product of Step B (80 mg, 0.11 mmol) as the appropriate amine, the desired product was obtained (73.1 mg, 73%).

HRMS-ESI (m/z) [M+H]⁺ calcd for C₅₂H₅₆F₂N₇O₅:896.4306, found 896.4311.

### Example 216: 1,2-dimethyl-5-[7-[(3R)-3-methyl-3,4-dihydro-1H-isoquinoline-2-carbonyl]-2-[2-[3-(1-methyl-4-piperidyl)phenyl]acetyl]-3,4-dihydro-1H-isoquinolin-6-yl]-N-(1-methylpyrazol-4-yl)-N-phenyl-pyrrole-3-carboxamide

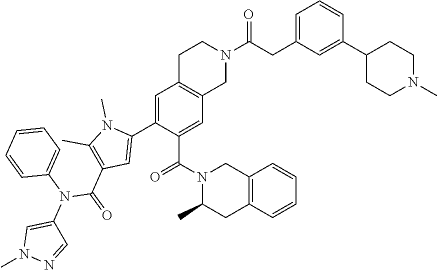

### Step A: tert-butyl 4-[3-(2-methoxy-2-oxo-ethyl)phenyl]-3,6-dihydro-2H-pyridine-1-carboxylate

Methyl 2-(3-bromophenyl)acetate (500 mg, 2.2 mmol), tert-butyl 4-(4,4,5,5-tetramethyl-1,3,2-dioxaborolan-2-yl)-3,6-dihydro-2H-pyridine-1-carboxylate (742 mg, 1.1 eq.) and potassium carbonate (905 mg, 3.0 eq.) were dissolved in a mixture of THF and water (10/1, 3.3 mL). The mixture was purged with nitrogen, then [1,1′-bis(diphenylphosphino)ferrocene]dichloropalladium (160 mg, 0.1 eq.) was added. Then, the reaction was heated to 110° C. and stirred for 0.5 h under microwave irradiation. Then the mixture was diluted with DCM (50 mL), washed with water (2×15 mL), the combined aqueous layers were extracted with DCM (1×15 mL), the combined organic layers were washed with brine (1×15 mL), dried over sodium sulphate, filtered and evaporated under reduced pressure. The crude product was purified by flash chromatography to yield the title product (381 mg, 52%).

¹H NMR (500 MHz, DMSO-d6) δ ppm 7.33 (m, 1H), 7.32 (dm, 1H), 7.29 (t, 1H), 7.16 (dm, 1H), 6.13 (br., 1H), 3.99 (br., 2H), 3.68 (s, 2H), 3.61 (s, 3H), 3.53 (t, 2H), 2.45 (br., 2H), 1.43 (s, 9H); ¹³C NMR (125 MHz, DMSO-d6) δ ppm 172.1, 154.4, 140.6, 134.9, 134.8, 128.9, 128.7, 126.3, 123.8, 121.6, 52.2, 43.8, 40.9/39.7, 40.6, 28.6, 27.1.

HRMS-ESI: (m/z) [M+Na]⁺ calcd for C₁₉H₂₅NNaO₄Na: 354.1676, found 354.1679.

### Step B: tert-butyl 4-[3-(2-methoxy-2-oxo-ethyl)phenyl]piperidine-1-carboxylate

The compound obtained in Step A (381 mg, 1.2 mmol) was dissolved in methanol (15 mL) and it was hydrogenated at atmospheric pressure using 24 mg of Pd catalyst (10% on charcoal) until complete conversion was observed. The mixture was filtered through a pad of Celite then it was concentrated under reduced pressure to afford the title compound (377 mg, 95%).

¹H NMR (500 MHz, DMSO-d6) δ ppm 7.24 (t, 1H), 7.13 (t, 1H), 7.13 (dt, 1H), 7.09 (dt, 1H), 4.07/2.79 (br+br, 4H), 3.65 (s, 2H), 3.61 (s, 3H), 2.65 (td, 1H), 1.73/1.46 (d+dd, 4H), 1.41 (s, 9H); ¹³C NMR (125 MHz, DMSO-d6) δ ppm 128.9, 128.3, 127.6, 125.7, 52.1, 44.3, 42, 40.6, 33.3, 28.6.

HRMS-ESI (m/z) [M+H]⁺ calcd for C₁₉H₂₇NNaO₄:356.1832, found 356.1835.

### Step C. methyl 2-[3-(4-piperidyl)phenyl]acetate

The compound obtained in Step B (357 mg, 1.1 mmol) was dissolved in 4 M HCl in dioxane (7 mL) and the reaction mixture was stirred until conversion was observed. The mixture was concentrated under reduced pressure and the crude product was purified by flash chromatography to afford the title compound (223 mg, 89%).

¹H NMR (500 MHz, DMSO-d6) δ ppm 7.23 (t, 1H), 7.11 (dt, 1H), 7.10 (t, 1H), 7.07 (dt, 1H), 3.65 (s, 2H), 3.61 (s, 3H), 3.00/2.56 (dt+td, 4H), 2.53 (td, 1H), 1.65/1.48 (d+dd, 4H); ¹³C NMR (125 MHz, DMSO-d6) δ ppm 128.8, 128.2, 127.3, 125.6, 52.2, 47.1, 43.0, 40.7, 34.7.

HRMS-ESI (m/z) [M+H]⁺ calcd for C₁₄H₂₀NO₂:234.1488, found 234.1489.

### Step D: 2-[3-(1-methyl-4-piperidyl)phenyl]acetic acid

To a solution of the compound obtained in Step C (213 mg, 0.91 mmol) in methanol (8 mL) was added formaldehyde (0.09 mL, 1.3 eq.) and acetic acid (0.9 mL 1.6 eq.) and the mixture was stirred at 60° C. for 30 min. Sodium cyanoborohydride (126 mg, 2.2 eq.) was added and the mixture was stirred at 60° C. for 2 h then it was poured into 20 mL of water and extracted with DCM (3×20 mL). The combined organic layers were washed with brine (1×20 mL), dried over magnesium sulphate, filtered and evaporated then purified by flash chromatography to provide the ester intermediate which was hydrolysed using General procedure 8 to afford the title product (108 mg, 48%)

¹H NMR (500 MHz, DMSO-d6) δ ppm 7.15 (t, 1H), 7.08 (dt, 1H), 7.04 (dt, 1H), 7.02 (t, 1H), 3.33 (s, 2H), 2.85/1.94 (dt+td, 4H), 2.38 (td, 1H), 2.18 (s, 3H), 1.69/1.63 (d+dd, 4H); ¹³C NMR (125 MHz, DMSO-d6) δ ppm 174.1, 146.1, 138.3, 128.2, 128.2, 127.5, 124.3, 56.3, 46.7, 44.4, 41.8, 33.5.

HRMS-ESI (m/z) [M+H]⁺ calcd for C₄H₂₀NO₂:234.1488, found 234.1488.

### Step E: 1,2-dimethyl-5-[7-[(3R)-3-methyl-3,4-dihydro-1H-isoquinoline-2-carbonyl]-2-[2-[3-(1-methyl-4-piperidyl)phenyl]acetyl]-3,4-dihydro-1H-isoquinolin-6-yl-N-(1-methylpyrazol-4-yl)-N-phenyl-pyrrole-3-carboxamide

Using General procedure 4c without the deprotection step, starting from the product of Step D as the appropriate acid (108 mg, 3.9 eq.) and the product obtained in Example 184, Step B (80 mg, 0.15 mmol) as the appropriate amine, the desired product was obtained (46 mg, 46%).

HRMS-ESI (m/z) [M+H]⁺ calcd for C₅₁H₅₆N₇O₃:814.4439, found 814.4443.

### Example 217: 1,2-dimethyl-5-[2-[2-[3-(6-methyl-2,6-diazaspiro[3.3]heptan-2-yl)phenyl]acetyl]-7-[(3R)-3-methyl-3,4-dihydro-1H-isoquinoline-2-carbonyl]-3,4-dihydro-1H-isoquinolin-6-yl]-N-(1-methylpyrazol-4-yl)-N-phenyl-pyrrole-3-carboxamide

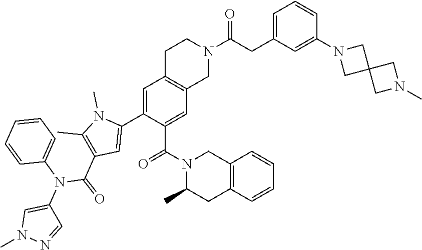

Using General procedure 4c without the deprotection step, starting from the product of Step A of Example 198 as the appropriate acid (46 mg, 1.5 eq.) and the product obtained in Example 184, Step B (75 mg, 0.13 mmol) as the appropriate amine, the desired product was obtained (72 mg, 68%).

HRMS-ESI (m/z) [M+H]⁺ calcd for C₅₁H₅₅N₈O₃:827.4392, found 827.4388.

### Example 218: N-(3-cyanophenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

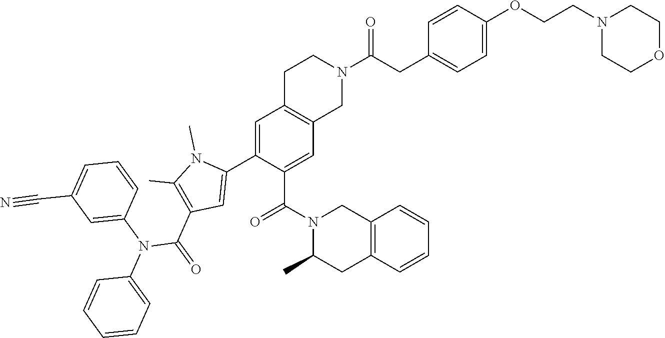 [SMILES: Cc1c(C(=O)N(c2ccccc2)c2cccc(C#N)c2)cc(-c2cc3c(cc2C(=O)N2Cc4ccccc4C[C@H]2C)CN(C(=O)Cc2ccc(OCCN4CCOCC4)cc2)CC3)n1C]

### Step A: 3-anilinobenzonitrile

Using General procedure 1b, starting from 3-bromobenzonitrile (1.0 g, 5.49 mmol) as the appropriate aryl bromide and aniline (768 mg, 1.5 eq.) afforded the title product (807 mg, 73%).

¹H NMR (500 MHz, DMSO-d6) δ ppm 8.55 (s, 1H), 7.39 (tm, 1H), 7.32 (dm, 1H), 7.32 (m, 1H), 7.3 (m, 2H), 7.18 (dm, 1H), 7.13 (dm, 2H), 6.95 (tm, 1H); ¹³C NMR (125 MHz, DMSO-d6) δ ppm 145.2, 142.2, 131.0, 129.9, 122.8, 121.9, 120.7, 119.6, 118.8, 118.1, 112.2.

HRMS-ESI (m/z) [M+H]⁺ calcd for C₁₃H₁₁N₂: 195.0917, found 195.0918.

### Step B: N-(3-cyanophenyl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.67-6.83 (m, 15H), 5.55-4.99 (s, 1H), 4.99-2.35 (m, 11H), 3.33-3.09 (s, 3H), 2.42-2.09 (s, 3H), 1.05/0.47 (d, 3H)

HRMS-ESI: (m/z) [M+H]⁺ calcd for C₄₀H₃₈N₅O₂: 620.3020, found 620.3024.

### Step C. N-(3-cyanophenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without deprotection, starting from Preparation Va as the appropriate acid (46 mg, 1.2 eq.) and the product of Step B (80 mg, 0.13 mmol) as the appropriate amine, the desired product was obtained (68 mg, 60%).

HRMS-ESI (m/z) [M+H]⁺ calcd for C₅₄H₅₅N₆O₅:867.4228, found 867.4227.

### Example 219: 5-[2-[2-[4-[2-(dimethylamino)ethoxy]-2-fluoro-phenyl]acetyl]-7-[(3R)-3-methyl-3,4-dihydro-1H-isoquinoline-2-carbonyl]-3,4-dihydro-1H-isoquinolin-6-yl]-N-(5-fluoro-6-methoxy-3-pyridyl)-1,2-dimethyl-N-phenyl-pyrrole-3-carboxamide

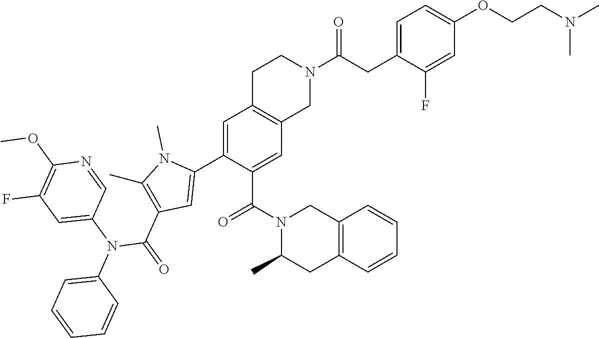

Using General procedure 4c, starting from the product obtained in Example 213, Step A as the appropriate acid (39 mg, 1.2 eq.) and the product of Example 183, Step B (87 mg, 0.14 mmol) as the appropriate amine, the desired product was obtained (55 mg, 46%).

HRMS-ESI (m/z) [M+H]⁺ calcd for C₅₁H₅₃F₂N₆O₅: 867.4040, found 867.4041.

### Example 220: 1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-(2-methylpyrazol-3-yl)-N-phenyl-pyrrole-3-carboxamide

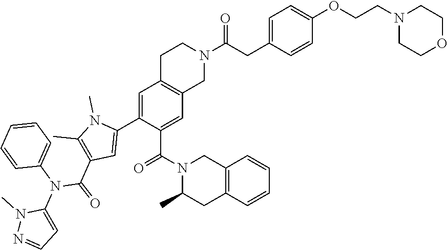 [SMILES: Cc1c(C(=O)N(c2ccccc2)c2ccnn2C)cc(-c2cc3c(cc2C(=O)N2Cc4ccccc4C[C@H]2C)CN(C(=O)Cc2ccc(OCCN4CCOCC4)cc2)CC3)n1C]

### Step A: 2-methyl-N-phenyl-pyrazol-3-amine

Using General procedure 1b, starting from 5-bromo-1-methyl-pyrazole (300 mg, 1.86 mmol) as the appropriate aryl bromide and aniline (260 mg, 1.5 eq.) afforded the title product (210 mg, 61%).

¹H NMR (500 MHz, DMSO-d6) δ ppm 7.94 (s, 1H), 7.34 (d, 1H), 7.19 (t, 2H), 6.82 (d, 2H), 6.76 (t, 1H), 5.97 (d, 1H), 3.63 (s, 3H); ¹³C NMR (125 MHz, DMSO-d6) δ ppm 145.2, 141.5, 138.1, 129.7, 119.4, 114.8, 96.3, 35.3.

HRMS-ESI (m/z) [M+H]⁺ calcd for C₁₀H₁₂N₃: 174.1026, found 174.1024.

### Step B: 1,2-dimethyl-5-[7-[(3R)-3-methyl-3,4-dihydro-1H-isoquinoline-2-carbonyl]-1,2,3,4-tetrahydroisoquinolin-6-yl]-N-(2-methylpyrazol-3-yl)-N-phenyl-pyrrole-3-carboxamide

The title compound was obtained using the method described in Preparation VIIb and the product from Step A as the appropriate aniline for the amide formation.

LRMS-ES: (m/z) [M+H]⁺ calcd for C₃₇H₃₉N₆O₂: 599.3, found 599.4.

### Step C. 1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-(2-methylpyrazol-3-yl)-N-phenyl-pyrrole-3-carboxamide

Using General procedure 4c without deprotection, starting from Preparation Va as the appropriate acid (17 mg, 1.5 eq.) and the product of Step B (26 mg, 0.04 mmol) as the appropriate amine, the desired product was obtained (32 mg, 87%).

HRMS-ESI (m/z) [M+H]⁺ calcd for C₅₁H₅₆N₇O₅:846.4337, found 846.4335.

### Example 221: 5-[2-[2-[4-[2-(dimethylamino)ethoxy]phenyl]acetyl]-7-[(3R)-3-methyl-3,4-dihydro-1H-isoquinoline-2-carbonyl]-3,4-dihydro-1H-isoquinolin-6-yl]-N-(1,3-dimethylpyrazol-4-yl)-1,2-dimethyl-N-phenyl-pyrrole-3-carboxamide

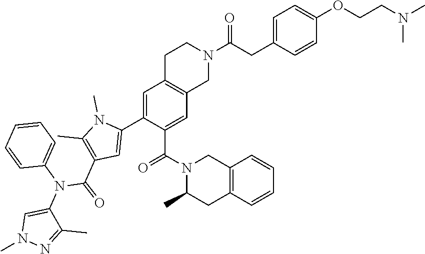

Using General procedure 4c, starting from the product obtained in Example 199, Step A as the appropriate acid (30 mg, 1.5 eq.) and the product of Example 187, Step B (55 mg, 0.09 mmol) as the appropriate amine, the desired product was obtained (38 mg, 52%).

HRMS-ESI (m/z) [M+H]⁺ calcd for C₅₀H₅₆N₇O₄:818.4388, found 818.4393.

### Example 222: 5-[2-[2-[4-[2-(dimethylamino)ethoxy]-2-fluoro-phenyl]acetyl]-7-[(3R)-3-methyl-3,4-dihydro-1H-isoquinoline-2-carbonyl]-3,4-dihydro-1H-isoquinolin-6-yl]-N-(1,3-dimethylpyrazol-4-yl)-1,2-dimethyl-N-phenyl-pyrrole-3-carboxamide

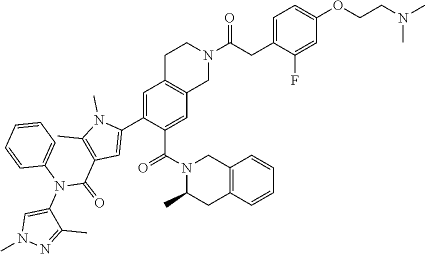

Using General procedure 4c, starting from the product obtained in Example 213, Step A as the appropriate acid (47 mg, 1.5 eq.) and the product of Example 187, Step B (80 mg, 0.13 mmol) as the appropriate amine, the desired product was obtained (80 mg, 71%).

HRMS-ESI (m/z) [M+Na]⁺ calcd for C₅₀H₅₄FN₇NaO₄Na: 858.4114, found 858.4113.

### Example 223: N-(5-cyano-1,2-dimethyl-pyrrol-3-yl)-5-[2-[2-[5-fluoro-2-methyl-4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

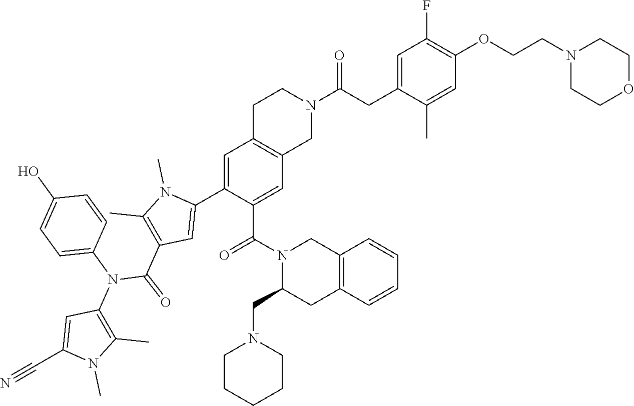 [SMILES: Cc1cc(OCCN2CCOCC2)c(F)cc1CC(=O)N1CCc2cc(-c3cc(C(=O)N(c4ccc(O)cc4)c4cc(C#N)n(C)c4C)c(C)n3C)c(C(=O)N3Cc4ccccc4C[C@H]3CN3CCCCC3)cc2C1]

### Step A: 2-(5-fluoro-4-hydroxy-2-methyl-phenyl)acetic acid

A 30 mL-microwave reaction vial was charged with 2-(5-fluoro-4-methoxy-2-methyl-phenyl)acetic acid (500 mg, 2.52 mmol) and 2M hydrogen bromide (12.6 mL, 10.0 eq.) then the mixture was heated at 150° C. under microwave irradiation for 5 min. The mixture was allowed to cool to rt and was poured into 150 mL of ice. The product was extracted with DCM (3×50 mL), the combined organic layers were dried over magnesium sulphate, filtered and evaporated (460 mg, 99%).

¹H NMR (500 MHz, DMSO-d6) δ ppm 12.26 (brs, 1H), 9.56 (s, 1H), 6.94 (d, 1H), 6.73 (d, 1H), 3.45 (s, 2H), 2.09 (s, 3H); ¹³C NMR (125 MHz, DMSO-d6) δ ppm 173.0, 149.2, 143.5, 133.2, 125.1, 119.5, 118.1, 38.1, 18.9.

HRMS-ESI (m/z) [M−H]⁺ calcd for C₉H₈FO₃:183.0463, found 183.0462.

### Step B: methyl 2-(5-fluoro-4-hydroxy-2-methyl-phenyl)acetate

The product obtained in Step A (460 mg, 2.50 mmol) was dissolved in methanol (12.5 mL) then concentrated hydrochloric acid (0.006 mL, 0.1 eq.) was added and the reaction mixture was stirred until complete conversion. The solvent was evaporated under reduced pressure to obtain the title product (480 mg. 97%).

¹H NMR (500 MHz, DMSO-d6) δ ppm 9.61 (br., 1H), 6.96 (d, 1H), 6.74 (d, 1H), 3.60 (s, 3H), 3.56 (s, 2H), 2.08 (s, 3H); ¹³C NMR (125 MHz, DMSO-d6) δ ppm 172.0, 149.3, 143.7, 133.3, 124.4, 119.6, 118.2, 52.1, 37.7, 18.9.

HRMS-ESI (m/z) [M+H]⁺ calcd for C₁₀H₁₂FO₃: 199.0765, found 199.0766.

### Step C: 2-[5-fluoro-2-methyl-4-(2-morpholinoethoxy)phenyl]acetic acid

Using the General procedure 7c and then General procedure 8, starting from the product obtained in Step B (0.48 g, 2.42 mmol) as the appropriate phenol and 2-morpholinoethanol (0.35 mL, 1.2 eq.) as the appropriate alcohol, the desired product was obtained (0.56 g, 78%).

¹H NMR (500 MHz, DMSO-d6) δ ppm 12.35 (br., 1H), 7.02 (d, 1H), 7 (d, 1H), 4.12 (t, 2H), 3.57 (t, 4H), 3.49 (s, 2H), 2.69 (t, 2H), 2.47 (t, 4H), 2.17 (s, 3H); ¹³C NMR (125 MHz, DMSO-d6) δ ppm 172.9, 150.0, 145, 133.5, 127.0, 118.1, 117.1, 67.1, 66.6, 57.4, 54.1, 38.2, 19.1.

HRMS-ESI (m/z) [M+H]⁺ calcd for C₁₅H₂₁FNO₄: 298.1449, found 298.1450.

### Step D: N-(5-cyano-1,2-dimethyl-pyrrol-3-yl)-5-[2-[2-[5-fluoro-2-methyl-4-(2-morpholinoethoxy)phenyl]acetyl]-7-[(3S)-3-(1-piperidylmethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

Using General procedure 4c, starting from the product of Step C (26 mg, 1.5 eq.) as the appropriate carboxylic acid and Preparation VIIa (50 mg, 0.06 mmol) as the appropriate amine, afforded the title compound (15 mg, 25%).

HRMS-ESI (m/z) [M+H]⁺ calcd for C₆₀H₆₈FN₈O₆: 1015.5240, found 1015.5238.

### Example 224: N-(5-cyano-1,2-dimethyl-pyrrol-3-yl)-5-[2-[2-[5-fluoro-2-methyl-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

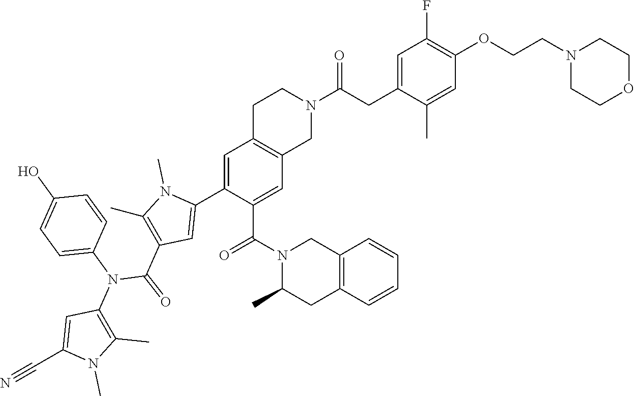 [SMILES: Cc1cc(OCCN2CCOCC2)c(F)cc1CC(=O)N1CCc2cc(-c3cc(C(=O)N(c4ccc(O)cc4)c4cc(C#N)n(C)c4C)c(C)n3C)c(C(=O)N3Cc4ccccc4C[C@H]3C)cc2C1]

Using General procedure 4c, starting from the product obtained in Example 223, Step C as the appropriate acid (47 mg, 1.5 eq.) and Preparation VIIb (80 mg, 0.10 mmol) as the appropriate amine, the desired product was obtained (55 mg, 50%).

HRMS-ESI (m/z) [M+H]⁺ calcd for C₅₅H₅₉FN₇O₆:932.4505, found 932.4500.

### Example 225: 5-[2-[2-[5-fluoro-2-methyl-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-(1-methylpyrazol-4-yl)-N-phenyl-pyrrole-3-carboxamide

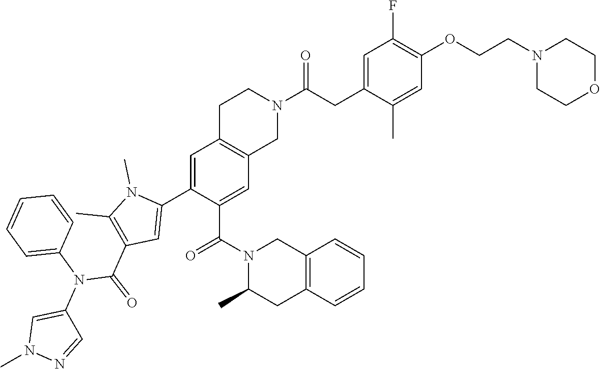 [SMILES: Cc1cc(OCCN2CCOCC2)c(F)cc1CC(=O)N1CCc2cc(-c3cc(C(=O)N(c4ccccc4)c4cnn(C)c4)c(C)n3C)c(C(=O)N3Cc4ccccc4C[C@H]3C)cc2C1]

Using General procedure 4c without deprotection, starting from the product obtained in Example 223, Step C as the appropriate acid (48 mg, 1.5 eq.) and the product obtained in Example 184, Step B (65 mg, 0.11 mmol) as the appropriate amine, the desired product was obtained (58 mg, 61%).

HRMS-ESI (m/z) [M+H]⁺ calcd for C₅₂H₅₇FN₇O₅:878.4400, found 878.4402.

### Example 226: N-(3-cyano-5-fluoro-phenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

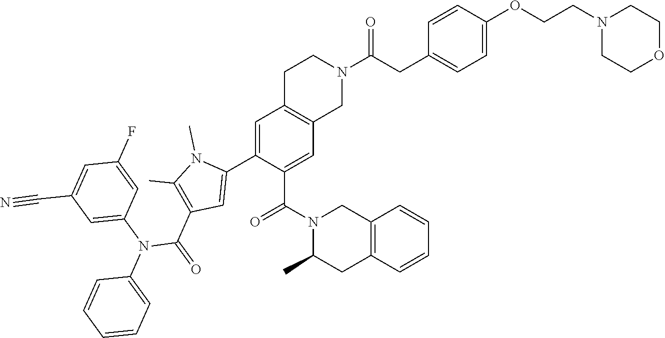 [SMILES: Cc1c(C(=O)N(c2ccccc2)c2cc(F)cc(C#N)c2)cc(-c2cc3c(cc2C(=O)N2Cc4ccccc4C[C@H]2C)CN(C(=O)Cc2ccc(OCCN4CCOCC4)cc2)CC3)n1C]

### Step A: 3-anilino-5-fluoro-benzonitrile

Using General procedure 1b, starting from 3-bromo-5-fluoro-benzonitrile (1.0 g, 5.0 mmol) as the appropriate aryl bromide and aniline (698 mg, 1.5 eq.) afforded the title product (786 mg, 72%).

¹H NMR (500 MHz, DMSO-d6) δ ppm 8.81 (s, 1H), 7.34 (t, 2H), 7.17 (d, 2H), 7.14 (t, 1H), 7.11 (dt, 1H), 7.05 (dt, 1H), 7.02 (t, 1H); ¹³C NMR (125 MHz, DMSO-d6) δ ppm 163.2, 147.5, 141.1, 130.0, 122.9, 119.9, 118.5, 114.8, 113.6, 108.9, 106.4.

HRMS-ESI (m/z) [M+H]⁺ calcd for C₁₃H₁₀FN₂: 213.0822, found 223.0822.

### Step B: N-(3-cyano-5-fluoro-phenyl)-1,2-dimethyl-5-[7-[(3R)-3-methyl-3,4-dihydro-1H-isoquinoline-2-carbonyl]-1,2,3,4-tetrahydroisoquinolin-6-yl]-N-phenyl-pyrrole-3-carboxamide

The title compound was obtained using the method described in Preparation VIIb and the product from Step A as the appropriate aniline for the amide formation.

¹H NMR (500 MHz, DMSO-d6) δ ppm 7.72-6.83 (m, 14H), 5.56-5.00 (s, 1H), 5.20-2.35 (m, 11H), 3.36-3.09 (s, 3H), 2.42-2.08 (s, 3H), 1.05-0.47 (d, 3H).

HRMS-ESI: (m/z) [M+H]⁺ calcd for C₄₀H₃₇FN₅O₂: 638.2926, found 638.2928.

### Step C. N-(3-cyano-5-fluoro-phenyl)-1,2-dimethyl-5-[7-[(3R)-3-methyl-3,4-dihydro-1H-isoquinoline-2-carbonyl]-2-[2-[4-(2-morpholinoethoxy)phenyl]acetyl]-3,4-dihydro-1H-isoquinolin-6-yl]-N-phenyl-pyrrole-3-carboxamide

Using General procedure 4c without deprotection, starting from Preparation Va as the appropriate acid (44 mg, 1.5 eq.) and the product of Step B (80 mg, 0.11 mmol) as the appropriate amine, the desired product was obtained (73 mg, 73%).

HRMS-ESI (m/z) [M+Na]⁺ calcd for C₅₄H₅₃FN₆NaO₅:907.3954, found 907.3953.

### Example 227: N-(3-cyano-5-fluoro-phenyl)-5-[2-[2-[2-fluoro-4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-1,2-dimethyl-N-phenyl-pyrrole-3-carboxamide

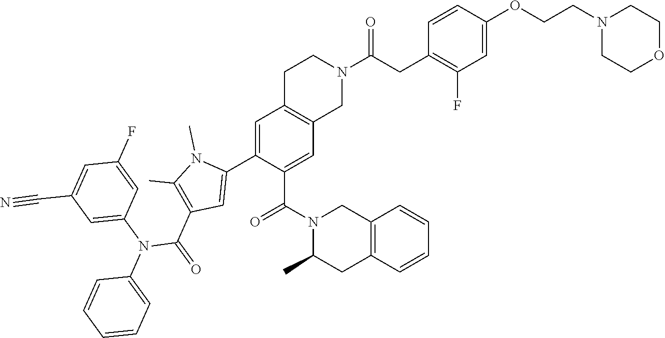 [SMILES: Cc1c(C(=O)N(c2ccccc2)c2cc(F)cc(C#N)c2)cc(-c2cc3c(cc2C(=O)N2Cc4ccccc4C[C@H]2C)CN(C(=O)Cc2ccc(OCCN4CCOCC4)cc2F)CC3)n1C]

Using General procedure 4c without deprotection, starting from the Preparation Vb as the appropriate acid (47 mg, 1.2 eq.) and the product obtained in Step B of Example 226 as the appropriate amine (80 mg, 0.13 mmol) as the appropriate amine, the desired product was obtained (37 mg, 33%).

HRMS-ESI (m/z) [M+Na]⁺ calcd for C₅₄H₅₂F₂N₆NaO₅: 925.3859, found 925.3855.

### Example 228: N-[1-(difluoromethyl)pyrazol-4-yl]-5-[2-[2-[4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

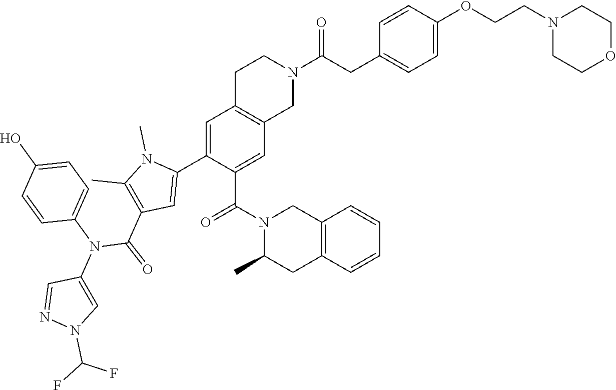 [SMILES: Cc1c(C(=O)N(c2ccc(O)cc2)c2cnn(C(F)F)c2)cc(-c2cc3c(cc2C(=O)N2Cc4ccccc4C[C@H]2C)CN(C(=O)Cc2ccc(OCCN4CCOCC4)cc2)CC3)n1C]

### Step A: N-[4-[tert-butyl(dimethyl)silyl]oxyphenyl]-1-(difluoromethyl)pyrazol-4-amine

Using General procedure 1b, starting from 4-bromo-]-(difluoromethyl)pyrazole (1.00 g, 5.08 mmol) as the appropriate aryl-bromide and Preparation IIa (1.19 g, 1.05 eq.) as the appropriate aniline, afforded the title compound (1.19 g, 69%).

¹H NMR (500 MHz, DMSO-d6) δ ppm 7.99 (s, 1H), 7.72 (s, 1H), 7.67 (t, 1H), 7.63 (s, 1H), 6.79 (d, 2H), 6.70 (d, 2H), 0.93 (s, 9H), 0.14 (s, 6H); ¹³C NMR (125 MHz, DMSO-d6) δ ppm 147.7, 139.7, 136.3, 129.6, 120.8, 115.5, 115.4, 111.3, 26.1, 18.4, −4.1.

HRMS-ESI (m/z) [M+H]⁺ calcd for C₁₆H₂₄F₂N₃OSi: 340.1651, found 340.1650.

### Step B: N-[1-(difluoromethyl)pyrazol-4-yl]-5-[2-[2-[4-(2-morpholinoethoxy)phenyl]acetyl]-7-[(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

Using General procedure 2a and Preparation VIa (175 mg, 0.25 mmol, 1 eq.) as the appropriate acid and the product from Step A (129 mg, 0.38 mmol, 1.5 eq.) as the appropriate aniline afforded an intermediate which was treated according to General procedure 3b to afford the title product (19.5 mg, 0.02 mmol, 9%).

HRMS-ESI (m/z) [M+H]⁺ calcd for C₅₁H₅₄F₂N₇O₆: 898.4104, found 898.4152.

The following compounds were prepared using procedures similar to those described above:

| | Structure | HRMS-ESI (m/z) |
|---|---|---|
| Example 229 | 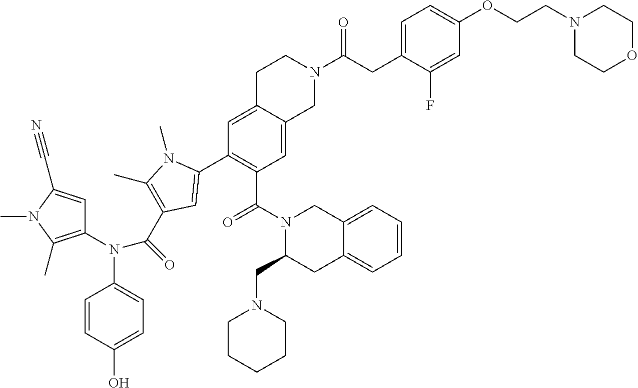 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCOCC5)cc3F)CC4)n(C)c2C)c2ccc(O)cc2)cc(C#N)n1C] | [M + H]⁺ calcd for C₅₉H₆₆FN₈O₆: 1001.5084; Found 1001.5072 |
| Example 230 | 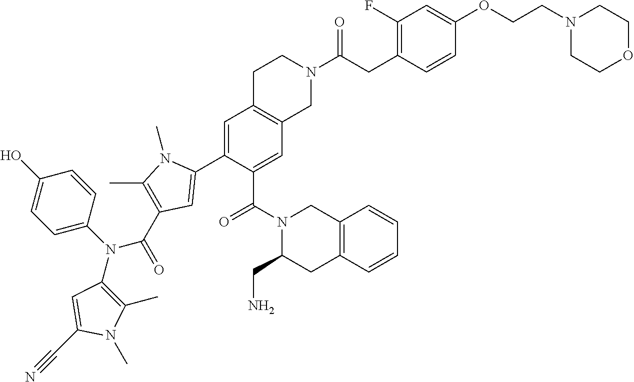 [SMILES: Cc1c(N(C(=O)c2cc(-c3cc4c(cc3C(=O)N3Cc5ccccc5C[C@H]3CN)CN(C(=O)Cc3ccc(OCCN5CCOCC5)cc3F)CC4)n(C)c2C)c2ccc(O)cc2)cc(C#N)n1C] | [M + H]⁺ calcd for C₅₄H₅₈FN₈O₆: 933.4458; Found 933.4461 |
| Example 231 | 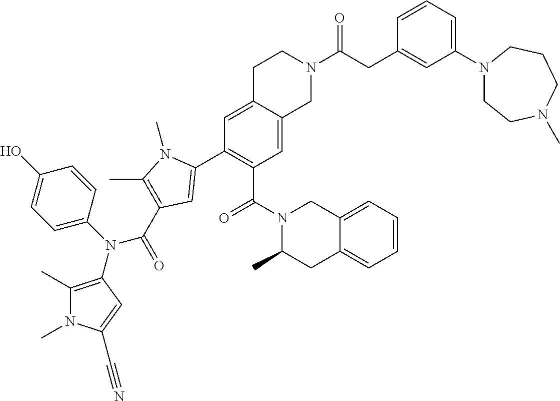 [SMILES: Cc1c(N(C(=O)c2cc(-c3cc4c(cc3C(=O)N3Cc5ccccc5C[C@H]3C)CN(C(=O)Cc3cccc(N5CCCN(C)CC5)c3)CC4)n(C)c2C)c2ccc(O)cc2)cc(C#N)n1C] | [M + H]⁺ calcd for C₅₄H₅₉N₈O₄: 883.4654; found 883.4651 |
| Example 232 | 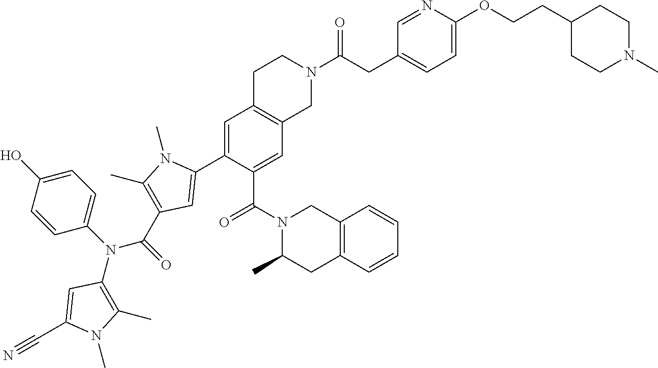 [SMILES: Cc1c(N(C(=O)c2cc(-c3cc4c(cc3C(=O)N3Cc5ccccc5C[C@H]3C)CN(C(=O)Cc3ccc(OCCC5CCN(C)CC5)nc3)CC4)n(C)c2C)c2ccc(O)cc2)cc(C#N)n1C] | [M + H]⁺ calcd for C₅₅H₆₁N₈O₅: 913.4759; found 913.4763 |
| Example 233 | 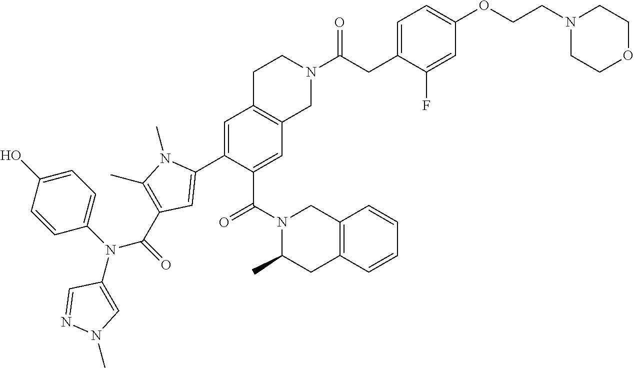 [SMILES: Cc1c(C(=O)N(c2ccc(O)cc2)c2cnn(C)c2)cc(-c2cc3c(cc2C(=O)N2Cc4ccccc4C[C@H]2C)CN(C(=O)Cc2ccc(OCCN4CCOCC4)cc2F)CC3)n1C] | [M + H]⁺ calcd for C₅₁H₅₅FN₇O₆: 880.4192; found 880.419 |
| Example 234 | 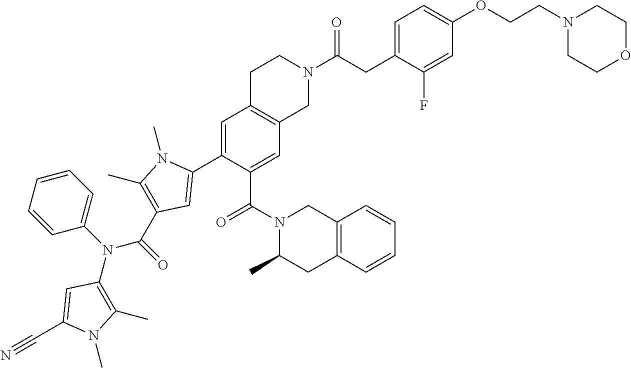 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3F)CC4)n(C)c2C)c2ccccc2)cc(C#N)n1C] | [M + H]⁺ calcd for C₅₄H₅₇FN₇O₅: 902.4400; Found 902.4400 |
| Example 235 | 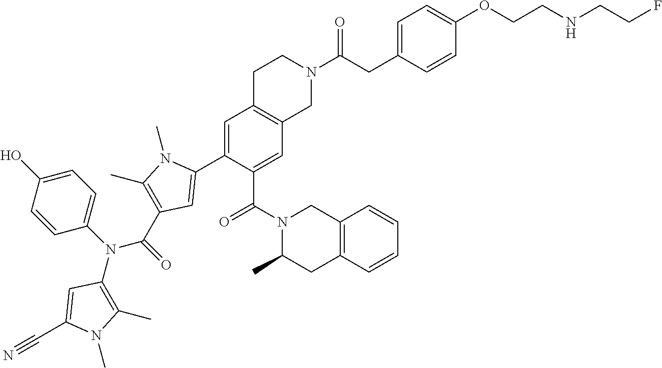 [SMILES: Cc1c(N(C(=O)c2cc(-c3cc4c(cc3C(=O)N3Cc5ccccc5C[C@H]3C)CN(C(=O)Cc3ccc(OCCNCCF)cc3)CC4)n(C)c2C)c2ccc(O)cc2)cc(C#N)n1C] | [M + H]⁺ calcd for C₅₂H₅₅FN₇O₅: 876.4243; found 876.4240 |
| Example 236 | 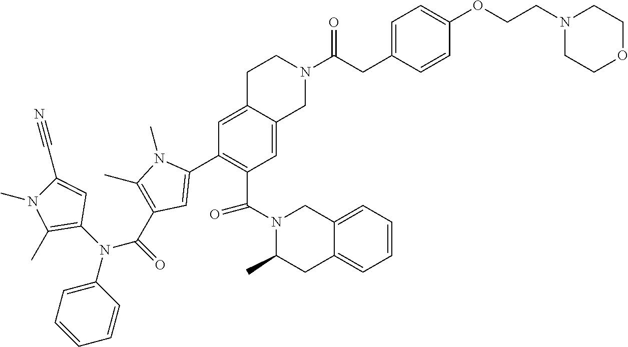 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cc(C#N)n1C] | [M + H]⁺ calcd for C₅₄H₅₈N₇O₅: 884.4494; found 884.4497 |
| Example 237 | 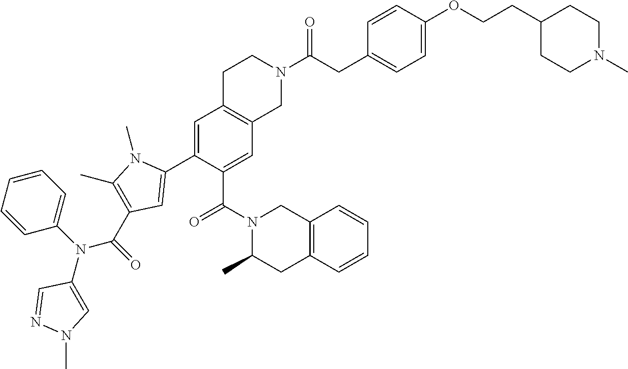 [SMILES: Cc1c(C(=O)N(c2ccccc2)c2cnn(C)c2)cc(-c2cc3c(cc2C(=O)N2Cc4ccccc4C[C@H]2C)CN(C(=O)Cc2ccc(OCCC4CCN(C)CC4)cc2)CC3)n1C] | [M + H]⁺ calcd for C₅₃H₆₀N₇O₄: 858.4701; found 858.4702 |
| Example 238 | 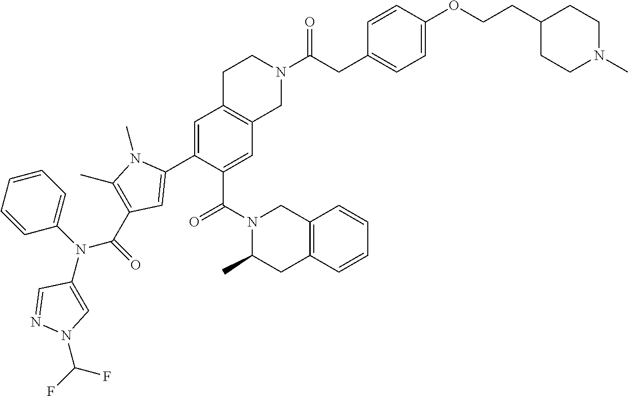 [SMILES: Cc1c(C(=O)N(c2ccccc2)c2cnn(C(F)F)c2)cc(-c2cc3c(cc2C(=O)N2Cc4ccccc4C[C@H]2C)CN(C(=O)Cc2ccc(OCCC4CCN(C)CC4)cc2)CC3)n1C] | [M + H]⁺ calcd for C₅₃H₅₈F₂N₇O₄: 894.4513; found 894.4517 |
| Example 239 | 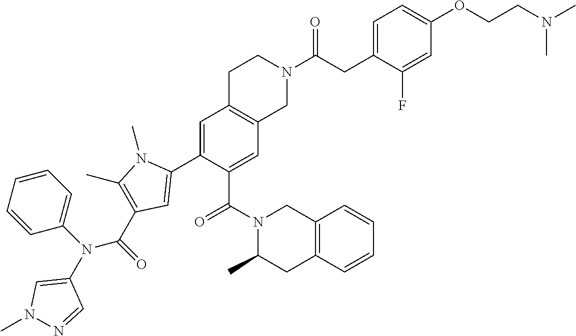 | [M + H]⁺ calcd for C₄₉H₅₃FN₇O₄: 822.4138; found 822.4142 |
| Example 240 | 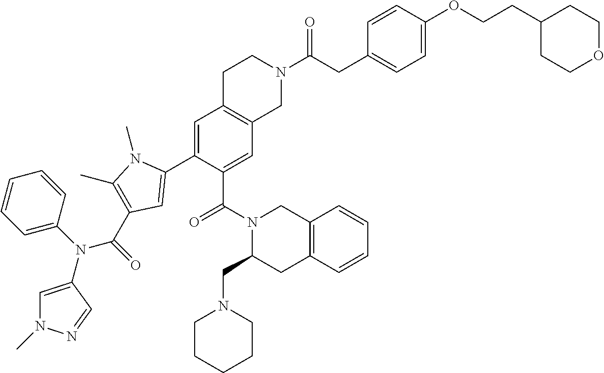 [SMILES: Cc1c(C(=O)N(c2ccccc2)c2cnn(C)c2)cc(-c2cc3c(cc2C(=O)N2Cc4ccccc4C[C@H]2CN2CCCCC2)CN(C(=O)Cc2ccc(OCCC4CCOCC4)cc2)CC3)n1C] | [M + H]⁺ calcd for C₅₇H₆₆N₇O₅: 928.5120; found 928.5123 |
| Example 241 | 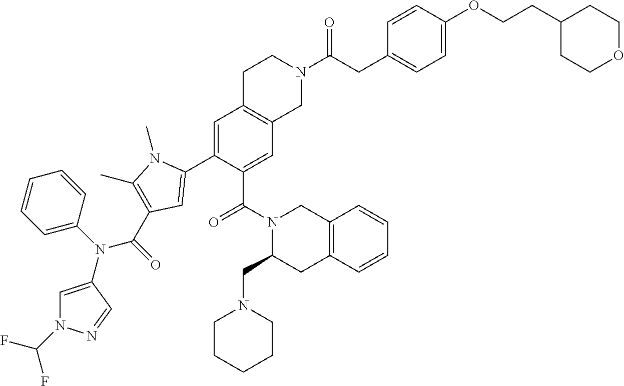 [SMILES: Cc1c(C(=O)N(c2ccccc2)c2cnn(C(F)F)c2)cc(-c2cc3c(cc2C(=O)N2Cc4ccccc4C[C@H]2CN2CCCCC2)CN(C(=O)Cc2ccc(OCCC4CCOCC4)cc2)CC3)n1C] | [M + H]⁺ calcd for C₅₇H₆₄F₂N₇O₅: 964.4932; found 964.4928 |

### Pharmacological Study

### Example A: BCL2 WT, BCL2 G101V, BCL2 D103Y, BCL2 F104I, BCL2 D111A and BCLXL WT TR-FRET Binding Assays

The relative binding potency of each compound was determined in a competitive binding assay using time resolved-fluorescence resonance energy transfer (TR-FRET). The method utilised a fluorescein labelled peptide derived from PUMA (5Flu-Ahx-QWAREIGAQLRRMADDLNAQYERR-NH2 wherein Ahx means 6-hexanoic acid monomer and the amino acid sequence is referred to as SEQ ID:01) which binds to a terbium labelled BCL2 or BCLXL protein. The degree of binding or corresponding compound inhibition can be quantified by the TR-FRET signal between the terbium donor and fluorescein acceptor.

The human BCL2_wild-type (also named BCL2_wt), BCL2_G101V mutant and BCLXL_wild-type were E. coli expressed as a N-terminal Halo-fusion proteins and singly labelled with Terbium on the Halo protein to afford the following amino acid sequences:

(i) Tb-Halo-(TEVcys)-huBCL2(1-207)-8His
(SEQ ID: 02):
AEIGTGFPFDPHYVEVLGERMHYVDVGPRDGTPVLFLHGNPTSSYVW
RNIIPHVAPTHRCIAPDLIGMGKSDKPDLGYFFDDHVRFMDAFIEAL
GLEEVVLVIHDWGSALGFHWAKRNPERVKGIAFMEFIRPIPTWDEWP
EFARETFQAFRTTDVGRKLIIDQNVFIEGTLPMGVVRPLTEVEMDHY
REPFLNPVDREPLWRFPNELPIAGEPANIVALVEEYMDWLHQSPVPK
LLFWGTPGVLIPPAEAARLAKSLPNCKAVDIGPGLNLLQEDNPDLIG
SEIARWLSTLEISGGGSENLYFQCMAHAGRTGYDNREIVMKYIHYKL
SQRGYEWDAGDVGAAPPGAAPAPGIFSSQPGHTPHPAASRDPVARTS
PLQTPAAPGAAAGPALSPVPPVVHLTLRQAGDDFSRRYRRDFAEMSS
QLHLTPFTARGRFATVVEELFRDGVNWGRIVAFFEFGGVMCVESVNR
EMSPLVDNIALWMTEYLNRHLHTWIQDNGGWDAFVELYGPSMRHHHH
HHHH
(ii) Tb-Halo-(TEVcys)-huBCL2(1-207)[G101V]-8His
(SEQ ID: 03)
AEIGTGFPFDPHYVEVLGERMHYVDVGPRDGTPVLFLHGNPTSSYVW
RNIIPHVAPTHRCIAPDLIGMGKSDKPDLGYFFDDHVRFMDAFIEAL
GLEEVVLVIHDWGSALGFHWAKRNPERVKGIAFMEFIRPIPTWDEWP
EFARETFQAFRTTDVGRKLIIDQNVFIEGTLPMGVVRPLTEVEMDHY
REPFLNPVDREPLWRFPNELPIAGEPANIVALVEEYMDWLHQSPVPK
LLFWGTPGVLIPPAEAARLAKSLPNCKAVDIGPGLNLLQEDNPDLIG
SEIARWLSTLEISGGGSENLYFQCMAHAGRTGYDNREIVMKYIHYKL
SQRGYEWDAGDVGAAPPGAAPAPGIFSSQPGHTPHPAASRDPVARTS
PLQTPAAPGAAAGPALSPVPPVVHLTLRQAVDDFSRRYRRDFAEMSS
QLHLTPFTARGRFATVVEELFRDGVNWGRIVAFFEFGGVMCVESVNR
EMSPLVDNIALWMTEYLNRHLHTWIQDNGGWDAFVELYGPSMRHHHH
HHHH
(iii) Tb-Halo-(TEVcys)-huBCL2(1-207)[D103Y]-
8His (SEQ ID: 04)
AEIGTGFPFDPHYVEVLGERMHYVDVGPRDGTPVLFLHGNPTSSYVW
RNIIPHVAPTHRCIAPDLIGMGKSDKPDLGYFFDDHVRFMDAFIEAL
GLEEVVLVIHDWGSALGFHWAKRNPERVKGIAFMEFIRPIPTWDEWP
EFARETFQAFRTTDVGRKLIIDQNVFIEGTLPMGVVRPLTEVEMDHY
REPFLNPVDREPLWRFPNELPIAGEPANIVALVEEYMDWLHQSPVPK
LLFWGTPGVLIPPAEAARLAKSLPNCKAVDIGPGLNLLQEDNPDLIG
SEIARWLSTLEISGGGSENLYFQCMAHAGRTGYDNREIVMKYIHYKL
SQRGYEWDAGDVGAAPPGAAPAPGIFSSQPGHTPHPAASRDPVARTS
PLQTPAAPGAAAGPALSPVPPVVHLTLRQAGDYFSRRYRRDFAEMSS
QLHLTPFTARGRFATVVEELFRDGVNWGRIVAFFEFGGVMCVESVNR
EMSPLVDNIALWMTEYLNRHLHTWIQDNGGWDAFVELYGPSMRHHHH
HHHH
(iv) Tb-Halo-(TEVcys)-huBCL2(1-207)[F104I]-
8His (SEQ ID: 05)
AEIGTGFPFDPHYVEVLGERMHYVDVGPRDGTPVLFLHGNPTSSYVW
RNIIPHVAPTHRCIAPDLIGMGKSDKPDLGYFFDDHVRFMDAFIEAL
GLEEVVLVIHDWGSALGFHWAKRNPERVKGIAFMEFIRPIPTWDEWP
EFARETFQAFRTTDVGRKLIIDQNVFIEGTLPMGVVRPLTEVEMDHY
REPFLNPVDREPLWRFPNELPIAGEPANIVALVEEYMDWLHQSPVPK
LLFWGTPGVLIPPAEAARLAKSLPNCKAVDIGPGLNLLQEDNPDLIG
SEIARWLSTLEISGGGSENLYFQCMAHAGRTGYDNREIVMKYIHYKL
SQRGYEWDAGDVGAAPPGAAPAPGIFSSQPGHTPHPAASRDPVARTS
PLQTPAAPGAAAGPALSPVPPVVHLTLRQAGDDISRRYRRDFAEMSS
QLHLTPFTARGRFATVVEELFRDGVNWGRIVAFFEFGGVMCVESVNR
EMSPLVDNIALWMTEYLNRHLHTWIQDNGGWDAFVELYGPSMRHHHH
HHHH
(v) Tb-Halo-(TEVcys)-huBCL2(1-207)[D111A]-
8His (SEQ ID: 06)
AEIGTGFPFDPHYVEVLGERMHYVDVGPRDGTPVLFLHGNPTSSYVW
RNIIPHVAPTHRCIAPDLIGMGKSDKPDLGYFFDDHVRFMDAFIEAL
GLEEVVLVIHDWGSALGFHWAKRNPERVKGIAFMEFIRPIPTWDEWP
EFARETFQAFRTTDVGRKLIIDQNVFIEGTLPMGVVRPLTEVEMDHY
REPFLNPVDREPLWRFPNELPIAGEPANIVALVEEYMDWLHQSPVPK
LLFWGTPGVLIPPAEAARLAKSLPNCKAVDIGPGLNLLQEDNPDLIG
SEIARWLSTLEISGGGSENLYFQCMAHAGRTGYDNREIVMKYIHYKL
SQRGYEWDAGDVGAAPPGAAPAPGIFSSQPGHTPHPAASRDPVARTS
PLQTPAAPGAAAGPALSPVPPVVHLTLRQAGDDFSRRYRRAFAEMSS
QLHLTPFTARGRFATVVEELFRDGVNWGRIVAFFEFGGVMCVESVNR
EMSPLVDNIALWMTEYLNRHLHTWIQDNGGWDAFVELYGPSMRHHHH
HHHH
(vi) Tb-Halo-(TEVcys)-huBCLXL(2-196)-8His
(SEQ ID: 07)
AEIGTGFPFDPHYVEVLGERMHYVDVGPRDGTPVLFLHGNPTSSYVW
RNIIPHVAPTHRCIAPDLIGMGKSDKPDLGYFFDDHVRFMDAFIEAL
GLEEVVLVIHDWGSALGFHWAKRNPERVKGIAFMEFIRPIPTWDEWP
EFARETFQAFRTTDVGRKLIIDQNVFIEGTLPMGVVRPLTEVEMDHY
REPFLNPVDREPLWRFPNELPIAGEPANIVALVEEYMDWLHQSPVPK
LLFWGTPGVLIPPAEAARLAKSLPNCKAVDIGPGLNLLQEDNPDLIG
SEIARWLSTLEISGGGSENLYFQCSQSNRELVVDFLSYKLSQKGYSW
SQFSDVEENRTEAPEGTESEMETPSAINGNPSWHLADSPAVNGATGH
SSSLDAREVIPMAAVKQALREAGDEFELRYRRAFSDLTSQLHITPGT
AYQSFEQVVNELFRDGVNWGRIVAFFSFGGALCVESVDKEMQVLVSR
IAAWMATYLNDHLEPWIQENGGWDTFVELYGHHHHHHHH

wherein the BCL2(1-207) sequence corresponds to UniProtKB® primary accession number P10415 and the BCLXL(2-196) sequence corresponds to UniProtKB® primary accession number Q07817-1.

The assay was performed in a 40 μL volume in white 384-standard well plates (Corning #3574). An 11-point serial dilution of each compound was prepared in DMSO and transferred directly to plate followed by addition of fluorescein labelled peptide and lastly protein. Final assay conditions were 0.5 nM BCL2_wt, BCL2_G101V, BCL2_D103Y, BCL2_F104I, BCL2_D111A or BCLXL_wt, 2 or 20 nM fluorescein labelled peptide, 50 mM Hepes pH 7.5, 150 mM NaCl, 0.5 mM TCEP (Tris(2-carboxyethyl)phosphine), 0.05% Tween 20, 5% DMSO. The mixture was incubated for 2 hours at 23° C.

TR-FRET measurements were performed on a Biotek Synergy Neo plate reader. TR-FRET was measured by excitation of the Terbium-donor at 340 nm and subsequent (delay time 100 μs) measurement of terbium and fluorescein emission at 495 nm and 520 nm, respectively, over a collection time of 300 μs. The TR-FRET signal was calculated as the emission-ratio at 520 nm over 495 nm.

The TR-FRET ratio readout for test compounds was normalized against 0% inhibition controls wells and 100% inhibition control wells. Test compound potency (IC₅₀) was estimated by nonlinear regression using the sigmoidal dose-response (variable slope) using Xlfit 4 (IDBS, Guildford, Surrey, UK, model 205). Were the IC₅₀ could not be determined the % inhibition at the highest tested concentration is given.

y=(A+((B−A)/(1+((C/x)\hat{ }D))))

where y is the normalized TR-TRET ratio measurement for a given concentration of test compound, x is the concentration of test compound, A is the estimated efficacy (% inhibition) at infinite compound dilution, and B is the maximal efficacy (% inhibition). C is the IC₅₀ value and D is the Hill slope coefficient.

The K₁ values were determined from the IC₅₀ values according to Cer et al, Nucleic Acids Res, 2009, July 1; 37 (WebServer issue): W441-W445.

The results are summarised in Table 1.

The compounds of the invention inhibited the interaction between the Bcl-2 protein (wild-type or one of the mutants selected from G101V, D103Y, and F104I) and the fluorescent peptide described hereinbefore. The compounds of the invention demonstrated an overall better on-target affinity for the Bcl-2 proteins as compared to Bcl-xL protein. In conclusion, the compounds showed a selective inhibition on Bcl-2 wild-type as well as on a panel of Bcl-2 mutants.

TABLE 1

| | | | | TR_FRET | TR_FRET | | | |
|---|---|---|---|---|---|---|---|---|
| | | TR_FRET | | EKI | EKI | TR_FRET | TR_FRET | TR_FRET |
| | TR_FRET | EKI_Bcl2 | TR_FRET | G101V/WT | BCLxL/WT | EKI_Bcl2 | EKI_Bcl2 | EKI_Bcl2 |
| Example | EKI BclxL | G101V | EKI_Bcl2 | selectivty | selectivty | D103Y | F104I | D111A |
| No | (uM) | (uM) | WT (uM) | ratio | ratio | (uM) | (uM) | (uM) |
| 1 | | 0.0022 | 0.00034 | 6.5 | N/A | | 0.00612 | |
| 2 | 0.0021 | 0.0005 | 0.00017 | 3.0 | 12.7 | 0.00109 | 0.00308 | |
| 3 | 0.0013 | 0.0004 | 0.00003 | 11.7 | 43.3 | 0.00012 | 0.00165 | |
| 4 | | 0.0032 | 0.00011 | 30.2 | N/A | | 0.01187 | |
| 5 | | 0.0013 | 0.00018 | 7.3 | N/A | | 0.00445 | |
| 6 | 0.0011 | 0.0007 | 0.00013 | 5.1 | 8.5 | 0.00067 | 0.00203 | |
| 7 | | 0.0008 | 0.00014 | 5.7 | N/A | 0.00088 | 0.00260 | |
| 8 | 0.0028 | 0.0012 | 0.00006 | 20.3 | 47.46 | | 0.00346 | |
| 9 | | 0.0028 | 0.00048 | 5.8 | N/A | 0.00127 | 0.01788 | |
| 10 | 0.0312 | 0.0057 | 0.00220 | 2.6 | 14.2 | 0.00298 | | |
| 11 | 0.0048 | 0.0013 | 0.00034 | 3.8 | 14.1 | 0.00055 | | |
| 12 | | 0.0017 | 0.00022 | 7.7 | N/A | 0.00040 | | |
| 13 | 0.0046 | 0.0020 | 0.00100 | 2.0 | 4.6 | 0.00235 | | |
| 14 | 0.053 | 0.0049 | 0.00090 | 5.4 | 58.9 | 0.00370 | | |
| 15 | 0.0084 | 0.0061 | 0.00067 | 9.1 | 12.5 | 0.00301 | | |
| 17 | 0.0096 | 0.0040 | 0.00065 | 6.2 | 14.8 | 0.00410 | 0.03386 | 0.00334 |
| 18 | 0.0302 | 0.0042 | 0.00030 | 14.0 | 100.7 | 0.00123 | 0.05355 | 0.00265 |
| 19 | 0.0074 | 0.0019 | 0.00040 | 4.8 | 18.5 | 0.00097 | | |
| 20 | 0.0066 | 0.0027 | 0.00053 | 5.0 | 12.5 | 0.00068 | | |
| 21 | 0.0011 | 0.0003 | 0.00011 | 3.1 | 10.0 | 0.00035 | 0.00029 | 0.00019 |
| 22 | 0.0023 | 0.0010 | 0.00020 | 5.0 | 11.5 | 0.00082 | | |
| 23 | 0.0485 | 0.0035 | 0.00058 | 6.0 | 83.6 | 0.00109 | 0.01973 | 0.00154 |
| 24 | 0.0061 | 0.0018 | 0.00053 | 3.4 | 11.5 | 0.00102 | | |
| 25 | 0.0032 | 0.0013 | 0.00017 | 7.6 | 18.8 | 0.00048 | | |
| 26 | 0.0232 | 0.0053 | 0.00110 | 4.8 | 21.1 | 0.00083 | | |
| 27 | 0.0101 | 0.0012 | 0.00080 | 1.5 | 12.6 | 0.00073 | 0.00236 | 0.00167 |
| 28 | 0.0056 | 0.0012 | 0.00025 | 4.8 | 22.4 | 0.00034 | 0.00647 | 0.00059 |
| 29 | 0.0131 | 0.0069 | 0.00067 | 10.3 | 19.6 | 0.00098 | 0.07803 | 0.00129 |
| 30 | 0.0011 | 0.0002 | 0.00010 | 1.9 | 11.6 | 0.00019 | 0.00026 | 0.00012 |
| 31 | 0.0013 | 0.0003 | 0.00011 | 2.6 | 11.8 | 0.00040 | 0.00052 | 0.00026 |
| 32 | 0.0067 | 0.0017 | 0.00064 | 2.6 | 10.5 | 0.00104 | | |
| 33 | 0.0037 | 0.0005 | 0.00015 | 3.3 | 24.7 | 0.00022 | 0.00050 | 0.00010 |
| 34 | 0.0053 | 0.0009 | 0.00034 | 2.6 | 15.6 | 0.00052 | 0.00027 | 0.00020 |
| 35 | 0.0026 | 0.0007 | 0.00026 | 2.6 | 10.0 | 0.00135 | 0.00077 | 0.00027 |
| 36 | 0.0014 | 0.0003 | 0.00011 | 2.5 | 12.7 | 0.00033 | 0.00016 | 0.00006 |
| 37 | 0.0013 | 0.0003 | 0.00010 | 2.7 | 13.0 | 0.00028 | | |
| 38 | 0.001 | 0.0005 | 0.00010 | 4.8 | 10.0 | 0.00032 | 0.00082 | 0.00015 |
| 39 | 0.0023 | 0.0007 | 0.00020 | 3.5 | 11.5 | 0.00071 | 0.00046 | 0.00018 |
| 40 | 0.007 | 0.0030 | 0.00068 | 4.4 | 10.3 | 0.00211 | | |
| 41 | 0.002 | 0.0004 | 0.00012 | 3.1 | 16.7 | 0.00055 | | |
| 42 | 0.003 | 0.0003 | 0.00018 | 1.8 | 16.7 | 0.00062 | 0.00076 | 0.00036 |
| 43 | 0.0022 | 0.0002 | 0.00004 | 5.0 | 55.0 | 0.00014 | 0.00016 | 0.00010 |
| 44 | 0.0022 | 0.0005 | 0.00006 | 8.5 | 36.1 | 0.00035 | 0.00134 | 0.00024 |
| 45 | 0.0025 | 0.0004 | 0.00008 | 4.8 | 31.6 | 0.00015 | 0.00165 | 0.00019 |
| 46 | 0.0015 | 0.0003 | 0.00014 | 2.4 | 10.7 | 0.00023 | 0.00035 | 0.00020 |
| 47 | 0.002 | 0.0002 | 0.00011 | 2.0 | 18.9 | 0.00024 | 0.00046 | 0.00016 |
| 48 | 0.0094 | 0.0013 | 0.00050 | 2.6 | 18.8 | 0.00079 | 0.01017 | 0.00163 |
| 49 | 0.0013 | 0.0002 | 0.00010 | 2.3 | 13.0 | 0.00017 | | |
| 50 | 0.0058 | 0.0006 | 0.00026 | 2.3 | 22.3 | 0.00058 | 0.00466 | 0.00146 |
| 51 | 0.0044 | 0.0010 | 0.00022 | 4.5 | 20.0 | 0.00092 | 0.00491 | 0.00089 |
| 52 | 0.003 | 0.0005 | 0.00020 | 2.3 | 15.2 | 0.00023 | 0.00077 | 0.00064 |
| 53 | 0.0012 | 0.0002 | 0.00007 | 2.8 | 18.5 | 0.00018 | 0.00035 | 0.00018 |
| 54 | 0.0023 | 0.0003 | 0.00010 | 2.7 | 24.0 | 0.00011 | 0.00052 | 0.00040 |
| 55 | 0.0026 | 0.0002 | 0.00010 | 2.4 | 26.5 | 0.00016 | 0.00030 | 0.00025 |
| 56 | 0.0016 | 0.0002 | 0.00006 | 2.8 | 26.7 | 0.00019 | 0.00028 | 0.00017 |
| 57 | 0.0017 | 0.0002 | 0.00005 | 4.1 | 34.7 | 0.00015 | 0.00047 | 0.00021 |
| 58 | 0.0023 | 0.0017 | 0.00021 | 8.1 | 11.0 | 0.00051 | | |
| 59 | 0.003 | 0.0007 | 0.00016 | 4.6 | 19.1 | 0.00040 | | |
| 60 | 0.001 | 0.0002 | 0.00005 | 3.5 | 19.2 | 0.00014 | 0.00021 | 0.00009 |
| 61 | 0.0015 | 0.0002 | 0.00004 | 5.0 | 35.7 | 0.00013 | 0.00102 | 0.00018 |
| 62 | 0.0024 | 0.0003 | 0.00006 | 5.5 | 38.7 | 0.00047 | 0.00038 | 0.00009 |
| 63 | 0.0017 | 0.0009 | 0.00011 | 7.8 | 15.5 | 0.00046 | 0.00284 | 0.00042 |
| 64 | 0.0028 | 0.0005 | 0.00011 | 4.5 | 25.5 | 0.00079 | | |
| 65 | 0.0041 | 0.0007 | 0.00008 | 9.4 | 52.6 | 0.00080 | 0.00112 | 0.00014 |
| 66 | 0.004 | 0.0005 | 0.00008 | 5.7 | 47.6 | 0.00023 | 0.00047 | 0.00023 |
| 67 | 0.0026 | 0.0002 | 0.00004 | 6.3 | 68.4 | 0.00016 | 0.00023 | 0.00009 |
| 68 | 0.0016 | 0.0004 | 0.00007 | 5.7 | 24.6 | 0.00023 | | |
| 69 | 0.0043 | 0.0020 | 0.00025 | 7.9 | 17.2 | 0.00050 | | |
| 70 | 0.0023 | 0.0009 | 0.00011 | 8.4 | 20.9 | 0.00029 | | |
| 71 | 0.0016 | 0.0012 | 0.00015 | 8.0 | 10.7 | 0.00068 | 0.00102 | 0.00031 |
| 72 | 0.0037 | 0.0009 | 0.00014 | 6.1 | 26.4 | 0.00037 | 0.00117 | 0.00021 |
| 73 | 0.0089 | 0.0027 | 0.00050 | 5.4 | 17.8 | 0.00174 | | |
| 74 | 0.0011 | 0.0001 | 0.00002 | 2.8 | 52.4 | 0.00007 | 0.00004 | 0.00009 |
| 75 | 0.0014 | 0.0005 | 0.00013 | 3.7 | 10.8 | 0.00028 | | |
| 76 | 0.0018 | 0.0003 | 0.00006 | 5.5 | 32.7 | 0.00018 | | |
| 77 | 0.008 | 0.0003 | 0.00006 | 5.1 | 135.6 | 0.00014 | 0.00047 | 0.00020 |
| 78 | 0.0071 | 0.0004 | 0.00005 | 6.8 | 134.0 | 0.00014 | 0.00043 | 0.00026 |
| 79 | 0.0039 | 0.0015 | 0.00026 | 5.8 | 15.0 | 0.00075 | | |
| 80 | 0.0012 | 0.0003 | 0.00006 | 4.9 | 21.1 | 0.00014 | | |
| 81 | 0.0053 | 0.0006 | 0.00019 | 2.9 | 27.9 | 0.00046 | 0.00052 | 0.00040 |
| 82 | 0.0063 | 0.0012 | 0.00019 | 6.7 | 33.9 | 0.00035 | | |
| 83 | 0.0093 | 0.0009 | 0.00009 | 10.9 | 109.4 | 0.00021 | | |
| 84 | 0.0018 | 0.0010 | 0.00016 | 5.9 | 11.3 | 0.00043 | | |
| 85 | 0.0098 | 0.0011 | 0.00032 | 3.3 | 30.6 | 0.00039 | | |
| 86 | 0.0016 | 0.0005 | 0.00013 | 3.9 | 12.3 | 0.00042 | 0.00037 | 0.00023 |
| 87 | 0.0013 | 0.0005 | 0.00012 | 4.2 | 10.8 | 0.00046 | | |
| 88 | 0.0017 | 0.0006 | 0.00010 | 5.9 | 17.0 | 0.00027 | | |
| 89 | 0.0019 | 0.0003 | 0.00007 | 4.3 | 29.2 | 0.00018 | 0.00043 | 0.00026 |
| 90 | 0.0045 | 0.0008 | 0.00025 | 3.4 | 18.0 | 0.00088 | | |
| 91 | 0.0024 | 0.0003 | 0.00004 | 6.8 | 60.0 | 0.00017 | 0.00025 | 0.00008 |
| 92 | 0.0085 | 0.0009 | 0.00003 | 30.7 | 303.6 | 0.00011 | | |
| 93 | 0.0024 | 0.0011 | 0.00019 | 5.5 | 12.6 | 0.00034 | | |
| 94 | 0.0033 | 0.0011 | 0.00022 | 5.0 | 15.0 | 0.00066 | | |
| 95 | 0.0039 | 0.0006 | 0.00018 | 3.2 | 21.7 | 0.00046 | | |
| 96 | 0.0024 | 0.0004 | 0.00006 | 5.6 | 37.5 | 0.00020 | 0.00020 | 0.00020 |
| 97 | 0.0019 | 0.0002 | 0.00005 | 4.3 | 41.3 | 0.00018 | 0.00016 | 0.00014 |
| 98 | 0.0047 | 0.0004 | 0.00007 | 4.9 | 66.2 | 0.00024 | 0.00060 | 0.00025 |
| 99 | 0.0028 | 0.0003 | 0.00005 | 5.2 | 52.8 | 0.00014 | | |
| 100 | 0.0023 | 0.0002 | 0.00002 | 8.9 | 127.8 | 0.00006 | | |
| 101 | 0.0019 | 0.0001 | 0.00002 | 4.7 | 105.6 | 0.00007 | | |
| 102 | 0.0028 | 0.0004 | 0.00006 | 6.5 | 49.1 | 0.00021 | 0.00073 | 0.00024 |
| 103 | 0.0011 | 0.0002 | 0.00003 | 6.5 | 35.5 | 0.00009 | | |
| 104 | 0.0026 | 0.0004 | 0.00007 | 5.8 | 40.0 | 0.00017 | | |
| 105 | 0.0016 | 0.0002 | 0.00005 | 4.3 | 32.7 | 0.00011 | 0.00022 | 0.00012 |
| 106 | 0.0018 | 0.0003 | 0.00005 | 6.2 | 36.0 | 0.00019 | | |
| 107 | 0.0078 | 0.0004 | 0.00009 | 4.3 | 87.6 | 0.00032 | 0.00034 | 0.00022 |
| 108 | 0.0021 | 0.0004 | 0.00007 | 5.1 | 30.9 | 0.00014 | 0.00019 | 0.00016 |
| 109 | 0.0039 | 0.0002 | 0.00004 | 5.5 | 90.7 | 0.00015 | 0.00025 | 0.00012 |
| 110 | 0.0041 | 0.0003 | 0.00004 | 6.3 | 102.5 | 0.00011 | 0.00015 | 0.00009 |
| 111 | 0.0072 | 0.0008 | 0.00015 | 5.2 | 48.0 | 0.00059 | 0.00079 | 0.00040 |
| 112 | 0.0071 | 0.0003 | 0.00005 | 5.6 | 134.0 | 0.0001 | | |
| 113 | 0.0036 | 0.0003 | 0.00005 | 5.2 | 69.2 | 0.00015 | 0.00027 | 0.00013 |
| 114 | 0.0043 | 0.0002 | 0.00005 | 3.9 | 84.3 | 0.00010 | | |
| 115 | 0.0028 | 0.0002 | 0.00003 | 5.6 | 100.0 | 0.00008 | 0.00030 | 0.00017 |
| 116 | 0.0033 | 0.0004 | 0.00005 | 7.0 | 61.1 | 0.00019 | | |
| 117 | 0.0017 | 0.0003 | 0.00005 | 5.3 | 36.2 | 0.00011 | 0.00018 | 0.00011 |
| 118 | 0.0052 | 0.0005 | 0.00010 | 5.1 | 52.5 | 0.00014 | | |
| 119 | 0.0022 | 0.0002 | 0.00004 | 5.6 | 56.4 | 0.00014 | | |
| 120 | 0.0053 | 0.0005 | 0.00010 | 5.2 | 53.0 | 0.00021 | 0.00037 | 0.00030 |
| 121 | 0.006 | 0.0005 | 0.00016 | 3.2 | 38.5 | 0.00034 | 0.00056 | 0.00023 |
| 122 | 0.0037 | 0.0003 | 0.00005 | 5.1 | 69.8 | 0.00013 | | |
| 123 | 0.0026 | 0.0003 | 0.00007 | 4.2 | 35.1 | 0.00028 | 0.00018 | 0.00013 |
| 124 | 0.0058 | 0.0007 | 0.00012 | 5.8 | 48.3 | 0.00058 | 0.00034 | 0.00028 |
| 125 | 0.0043 | 0.0004 | 0.00007 | 5.4 | 61.4 | 0.00019 | 0.00017 | 0.00013 |
| 126 | 0.0077 | 0.0007 | 0.00012 | 5.4 | 64.2 | 0.00037 | 0.00049 | 0.00038 |
| 127 | 0.0036 | 0.0002 | 0.00005 | 4.3 | 76.6 | 0.00016 | 0.00026 | 0.00013 |
| 128 | 0.0054 | 0.0004 | 0.00006 | 6.9 | 93.1 | 0.00021 | | |
| 129 | 0.0144 | 0.0008 | 0.00015 | 5.5 | 96.0 | 0.00024 | | |
| 130 | 0.0061 | 0.0023 | 0.00040 | 5.8 | 15.3 | 0.00133 | | |
| 131 | 0.0087 | 0.0015 | 0.00023 | 6.5 | 37.8 | 0.00053 | | |
| 132 | 0.012 | 0.0029 | 0.00014 | 20.4 | 85.7 | 0.00037 | | |
| 133 | 0.0019 | 0.0006 | 0.00007 | 9.4 | 29.2 | 0.00038 | | |
| 134 | 0.0026 | 0.0003 | 0.00006 | 5.8 | 45.6 | 0.00018 | 0.00033 | 0.00017 |
| 135 | 0.0045 | 0.0004 | 0.00007 | 5.8 | 60.8 | 0.00021 | 0.00034 | 0.00017 |
| 136 | 0.0023 | 0.0006 | 0.00013 | 4.2 | 17.7 | 0.00032 | 0.00017 | 0.00035 |
| 137 | 0.0014 | 0.0003 | 0.00006 | 4.6 | 23.7 | 0.00016 | | |
| 138 | 0.0022 | 0.0005 | 0.00008 | 6.2 | 27.2 | 0.00024 | | |
| 139 | 0.0057 | 0.0010 | 0.00012 | 8.3 | 47.5 | 0.00051 | | |
| 140 | 0.0028 | 0.0004 | 0.00008 | 5.1 | 35.9 | 0.00017 | | |
| 141 | 0.0011 | 0.0003 | 0.00006 | 4.7 | 17.7 | 0.00020 | | |
| 142 | 0.0081 | 0.0013 | 0.00013 | 10.0 | 62.3 | 0.00031 | | |
| 143 | 0.003 | 0.0004 | 0.00004 | 12.2 | 83.3 | 0.00010 | | |
| 144 | 0.0077 | 0.0011 | 0.00010 | 11.0 | 77.0 | 0.00018 | | |
| 145 | 0.0011 | 0.0002 | 0.00003 | 5.4 | 39.3 | 0.00011 | 0.00014 | 0.00007 |
| 146 | 0.0035 | 0.0003 | 0.00004 | 7.2 | 81.4 | 0.00012 | 0.00027 | 0.00015 |
| 147 | 0.001 | 0.0003 | 0.00006 | 5.9 | 17.2 | 0.00014 | | |
| 148 | 0.0015 | 0.0003 | 0.00006 | 4.2 | 25.0 | 0.00018 | 0.00029 | 0.00015 |
| 149 | 0.0019 | 0.0011 | 0.00010 | 11.2 | 19.4 | 0.00023 | | |
| 150 | 0.0026 | 0.0002 | 0.00004 | 6.2 | 70.3 | 0.00008 | | |
| 151 | 0.0027 | 0.0003 | 0.00005 | 6.4 | 57.4 | 0.00010 | | |
| 152 | 0.0039 | 0.0011 | 0.00016 | 6.9 | 24.4 | 0.00041 | 0.00064 | 0.00036 |
| 153 | 0.0052 | 0.0004 | 0.00013 | 3.5 | 41.6 | 0.00038 | | |
| 154 | 0.0041 | 0.0003 | 0.00004 | 8.4 | 110.8 | 0.00014 | | |
| 155 | 0.0017 | 0.0004 | 0.00004 | 8.8 | 39.5 | 0.00011 | | |
| 156 | 0.0027 | 0.0002 | 0.00005 | 4.0 | 50.9 | 0.00010 | | |
| 157 | 0.002 | 0.0003 | 0.00004 | 7.7 | 45.5 | 0.00009 | | |
| 158 | 0.0054 | 0.0009 | 0.00010 | 9.4 | 54.0 | 0.00047 | | |
| 159 | 0.0037 | 0.0003 | 0.00005 | 5.1 | 69.8 | 0.00013 | | |
| 160 | 0.0015 | 0.0002 | 0.00003 | 6.3 | 55.6 | 0.00018 | | |
| 161 | 0.0029 | 0.0003 | 0.00005 | 7.0 | 61.7 | 0.00014 | | |
| 162 | 0.0092 | 0.0006 | 0.00013 | 4.8 | 70.8 | 0.00030 | | |
| 163 | 0.0083 | 0.0008 | 0.00016 | 5.3 | 51.9 | 0.00064 | | |
| 164 | 0.0093 | 0.0006 | 0.00008 | 7.5 | 120.8 | 0.00013 | | |
| 165 | 0.01 | 0.0008 | 0.00004 | 21.1 | 277.8 | 0.00013 | | |
| 166 | 0.0027 | 0.0003 | 0.00004 | 5.8 | 62.8 | 0.00016 | | |
| 167 | 0.0084 | 0.0003 | 0.00005 | 6.5 | 171.4 | 0.00015 | | |
| 168 | 0.011 | 0.0005 | 0.00011 | 4.7 | 100.0 | 0.00022 | | |
| 169 | 0.0012 | 0.0004 | 0.00009 | 4.6 | 12.8 | 0.00036 | | |
| 170 | 0.0045 | 0.0013 | 0.00030 | 4.3 | 15.0 | 0.00106 | | |
| 171 | 0.0013 | 0.0002 | 0.00002 | 7.1 | 61.9 | 0.00009 | | |
| 172 | 0.0032 | 0.0005 | 0.00011 | 4.4 | 29.1 | 0.00039 | | |
| 173 | 0.004 | 0.0003 | 0.00003 | 9.0 | 133.3 | 0.00012 | | |
| 174 | 0.0026 | 0.0005 | 0.00007 | 7.8 | 38.8 | 0.00030 | | |
| 175 | 0.0031 | 0.0005 | 0.00008 | 7.0 | 40.8 | 0.00034 | | |
| 176 | 0.0023 | 0.0002 | 0.00003 | 5.8 | 74.2 | 0.00016 | | |
| 177 | 0.0124 | 0.0005 | 0.00005 | 10.0 | 229.0 | 0.00012 | | |
| 178 | 0.0119 | 0.0013 | 0.00009 | 15.3 | 137.8 | 0.00016 | | |
| 179 | 0.0061 | 0.0006 | 0.00004 | 14.7 | 148.0 | 0.00013 | | |
| 180 | 0.0172 | 0.0003 | 0.00007 | 4.5 | 252.5 | 0.00015 | | |
| 181 | 0.0090 | 0.0004 | 0.00004 | 9.9 | 248.7 | 0.00009 | | |
| 182 | 0.0118 | 0.0015 | 0.00011 | 13.3 | 107.1 | 0.00030 | | |
| 183 | 0.0151 | 0.0056 | 0.00013 | 43.8 | 117.3 | 0.00035 | | |
| 184 | 0.0123 | 0.0020 | 0.00003 | 66.2 | 411.3 | 0.00009 | | |
| 185 | 0.0059 | 0.0015 | 0.00006 | 23.4 | 92.8 | 0.00013 | | |
| 186 | 0.0053 | 0.0002 | 0.00001 | 13.2 | 410.0 | 0.00004 | | |
| 187 | 0.0173 | 0.0027 | 0.00003 | 78.9 | 507.9 | 0.00008 | | |
| 188 | 0.0239 | 0.0022 | 0.00003 | 71.6 | 769.6 | 0.00009 | | |
| 189 | 0.0334 | 0.0174 | 0.00019 | 92.0 | 176.8 | 0.00199 | | |
| 190 | 0.0168 | 0.0036 | 0.00005 | 73.4 | 342.7 | 0.00012 | | |
| 191 | 0.0095 | 0.0024 | 0.00004 | 65.3 | 264.0 | 0.00007 | | |
| 192 | 0.0077 | 0.0026 | 0.00004 | 58.9 | 175.0 | 0.00011 | | |
| 193 | 0.0110 | 0.0031 | 0.00004 | 72.0 | 256.9 | 0.00010 | | |
| 194 | 0.0033 | 0.0010 | 0.00003 | 35.7 | 115.0 | 0.00008 | | |
| 195 | 0.0050 | 0.0009 | 0.000014 | 63.6 | 359.1 | 0.00005 | | |
| 196 | 0.0096 | 0.0027 | 0.00003 | 83.2 | 301.1 | 0.00009 | | |
| 197 | 0.0099 | 0.0012 | 0.00002 | 63.9 | 523.3 | 0.00005 | | |
| 198 | 0.0006 | 0.0014 | 0.00001 | 138.7 | 58.6 | 0.00007 | | |
| 199 | 0.0088 | 0.0046 | 0.00008 | 60.1 | 116.3 | 0.00019 | | |
| 200 | 0.0260 | 0.0055 | 0.00006 | 88.0 | 419.8 | 0.00015 | | |
| 201 | 0.0164 | 0.0012 | 0.00005 | 23.3 | 322.5 | 0.00009 | | |
| 202 | 0.0109 | 0.0012 | 0.00005 | 22.9 | 214.7 | 0.00011 | | |
| 203 | 0.0085 | 0.0005 | 0.00002 | 23.3 | 404.3 | 0.00006 | | |
| 204 | 0.0096 | 0.0019 | 0.00006 | 30.8 | 152.4 | 0.00017 | | |
| 205 | 0.0200 | 0.0074 | 0.00007 | 100.3 | 270.3 | 0.00019 | | |
| 206 | 0.1070 | 0.0045 | 0.000014 | 321.5 | 7642.9 | 0.00009 | | |
| 207 | 0.0030 | 0.0014 | 0.000020 | 68.8 | 150.0 | 0.00004 | | |
| 208 | 0.0088 | 0.0037 | 0.00004 | 83.8 | 200.0 | 0.00012 | | |
| 209 | 0.0230 | 0.0038 | 0.00008 | 47.5 | 291.1 | 0.00021 | | |
| 210 | 0.0290 | 0.0041 | 0.00004 | 118.3 | 828.6 | 0.00011 | | |
| 211 | 0.0260 | 0.0053 | 0.00007 | 72.1 | 351.4 | 0.00018 | | |
| 212 | 0.0048 | 0.0017 | 0.00005 | 37.0 | 104.3 | 0.00013 | | |
| 213 | 0.0010 | 0.0004 | 0.00001 | 46.6 | 108.9 | 0.00003 | | |
| 214 | 0.0086 | 0.0007 | 0.00005 | 13.3 | 168.6 | 0.00012 | | |
| 215 | 0.0160 | 0.0046 | 0.00006 | 80.9 | 280.7 | 0.00013 | | |
| 216 | 0.0026 | 0.0064 | 0.00005 | 125.5 | 50.1 | 0.00033 | | |
| 217 | 0.0006 | 0.0020 | 0.00002 | 88.7 | 24.9 | 0.00009 | | |
| 218 | 0.0138 | 0.0015 | 0.00003 | 45.3 | 418.1 | 0.00008 | | |
| 219 | 0.0029 | 0.0046 | 0.00005 | 89.8 | 57.8 | 0.00013 | | |
| 220 | 0.0423 | 0.0143 | 0.00013 | 109.9 | 325.6 | 0.00037 | | |
| 221 | 0.0283 | 0.0191 | 0.00024 | 79.5 | 118.0 | 0.00053 | | |
| 222 | 0.0125 | 0.0063 | 0.00009 | 69.6 | 138.9 | 0.00019 | | |
| 223 | 0.0043 | 0.0016 | 0.00018 | 9.3 | 24.6 | 0.00026 | | |
| 224 | 0.0013 | 0.0008 | 0.000073 | 10.5 | 17.7 | 0.00018 | | |
| 225 | 0.0236 | 0.0256 | 0.00011 | 233.0 | 214.3 | 0.00014 | | |
| 226 | 0.0418 | 0.0084 | 0.00026 | 32.2 | 160.8 | 0.00076 | | |
| 227 | 0.0372 | 0.0108 | 0.00017 | 63.7 | 219.1 | 0.00033 | | |
| 228 | 0.0149 | 0.00084 | 0.00006 | 14 | 248.3 | 0.00021 | 0.0016 | 0.0002 |
| 229 | 0.0018 | 0.00022 | 0.00004 | 5.5 | 45 | 0.00011 | 0.000164 | 0.0001 |
| 230 | 0.0009 | 0.000067 | 0.000014 | 4.8 | 64.3 | 0.000066 | | |
| 233 | 0.00274 | 0.00032 | 0.000039 | 8.2 | 70.3 | 0.00008 | | |
| 234 | 0.00368 | 0.00076 | 0.000086 | 8.9 | 42.8 | 0.00016 | | |
| 235 | 0.00089 | 0.00013 | 0.000031 | 4.2 | 28.7 | 0.00011 | | |
| 236 | 0.00415 | 0.00073 | 0.000078 | 9.3 | 53.1 | 0.000786 | 0.00112 | 0.000132 |
| 237 | 0.0017 | 0.00052 | 0.000032 | 16.3 | 53.1 | 0.000057 | | |
| 238 | 0.0028 | 0.00107 | 0.000064 | 16.7 | 43.8 | 0.00013 | | |
| 239 | 0.0043 | 0.00525 | 0.000066 | 79.5 | 65.2 | 0.000132 | | |
| 240 | 0.0375 | 0.0067 | 0.000116 | 57.8 | 323 | 0.00015 | | |
| 241 | 0.0527 | 0.0392 | 0.000244 | 160.7 | 216 | 0.00064 | | |

N/A: not applicable

### Example B: In Vitro Cytotoxicity of the Bcl-2 Inhibitors in Modified Cells Expressing Either Bcl-2 Wild-Type (KMS-12-PE WT) or Bcl-2 Gly101Val Mutant (KMS-12-PE

### Material and Methods

KMS-12-PE (ACC 606) were purchased from the Leibniz-Institute DSMZ (Braunschweig, Germany) and were grown at 37° C. in a humidified atmosphere with 500 CO2 in media recommended by the suppliers. Lentiviral particles containing Bcl-2 wild type (also named “Bcl2 WT”) and Bcl-2 mutated on G101V (also named “Bcl2 G101V”) were cloned into pcLV-CMV-DEST-IRES-TagRFP. Lentiviral particles (1×106) were mixed with Polybrene at 8 g/ml and transduced by spinoculation for 1 h at 32° C. and incubated overnight. After 8 days, TagRFP positive-cells were sorted by FACS. BCL2 expression was monitored by immunoblotting using anti-Flag and anti-BCL2 antibodies.

Cellular viability was monitored with the cell viability assay (CellTiter-Glo®) on KMS-12-PE WT and G101V cell lines. The CellTiter-Glo® (CTG) Luminescent Cell Viability Assay (from Promega) is a homogeneous method to determine the number of viable cells in culture based on quantitation of the ATP present, which signals the presence of metabolically active cells.

Dose response of compounds are performed with 10 mM stock solutions (DMSO), 11-points curve, in a 384-well compound plate. The assay plate is prepared by stamping 40 nL from compound dilution plate of all different concentrations (e.g. 10 mM to have final 10 pM assay test) into a cell culture microplate, using Echo acoustic liquid handling (Labcyte, Beckman). 40 μl of cell solution is added and cells are incubated with compounds for 48 h at 37° C., 5% CO2. CTG was added to the cells (according to the manufacturer instructions) and luminescence intensity was recorded using the Pherastar plate reader (BMG).

This assay provides in vitro cellular potency of the tested Bcl-2 inhibitors, measuring the C50, defined as the concentration needed to reach 50% of the cellular viability.

The results are summarised in Table 2. They show that the compounds of the invention inhibited cell viability in cell lines overexpressing Bcl-2 wild-type or Bcl-2 G101V. The compounds of the invention demonstrated a cytotoxic profile in the previous cell lines.

TABLE 2

| | KMS-12-PE | KMS-12-PE | |
|---|---|---|---|
| Example | Bcl-2 WT | Bcl-2 G101V | Ratio |
| No | (C50; uM) | (C50; uM) | (G101V/WT) |
| 1 | 0.0087 | 0.0776 | 8.92 |
| 2 | 0.004 | 0.02 | 5.04 |
| 3 | 0.0054 | 0.051 | 9.44 |
| 4 | 0.0048 | 0.124 | 25.9 |
| 5 | 0.0057 | 0.0285 | 4.98 |
| 6 | 0.0106 | 0.0566 | 5.34 |
| 7 | 0.0058 | 0.0713 | 12.2 |
| 8 | 0.0026 | 0.0727 | 27.6 |
| 9 | 0.0254 | 0.178 | 7.01 |
| 10 | 0.014 | 0.0641 | 4.58 |
| 11 | 0.0035 | 0.0249 | 7.05 |
| 12 | 0.0074 | 0.0676 | 9.14 |
| 13 | 0.0131 | 0.0704 | 5.37 |
| 14 | 0.0096 | 0.0852 | 8.86 |
| 15 | 0.0166 | 0.0714 | 4.3 |
| 17 | 0.0032 | 0.0144 | 4.51 |
| 18 | 0.0055 | 0.0477 | 8.67 |
| 19 | 0.0061 | 0.0423 | 6.91 |
| 20 | 0.0101 | 0.0681 | 6.74 |
| 21 | 0.0057 | 0.023 | 4.02 |
| 22 | 0.0102 | 0.0864 | 8.47 |
| 23 | 0.0096 | 0.0898 | 9.34 |
| 24 | 0.0059 | 0.0423 | 7.19 |
| 25 | 0.0063 | 0.0778 | 12.4 |
| 26 | N.D | N.D | N.D |
| 27 | 0.0037 | 0.0143 | 4.23 |
| 28 | 0.0099 | 0.0643 | 6.51 |
| 29 | 0.0164 | 0.0594 | 3.62 |
| 30 | 0.0191 | 0.0909 | 4.76 |
| 31 | 0.0077 | 0.0312 | 4.05 |
| 32 | 0.0073 | 0.0393 | 5.38 |
| 33 | 0.0054 | 0.0491 | 9.09 |
| 34 | 0.0052 | 0.0275 | 5.29 |
| 35 | 0.0068 | 0.0254 | 3.75 |
| 36 | 0.0103 | 0.0386 | 3.75 |
| 37 | 0.0174 | 0.0932 | 5.36 |
| 38 | 0.0091 | 0.0509 | 5.57 |
| 39 | 0.0056 | 0.023 | 4.07 |
| 40 | 0.0094 | 0.0426 | 4.54 |
| 41 | 0.0094 | 0.051 | 5.41 |
| 42 | 0.0046 | 0.0175 | 3.77 |
| 43 | 0.0048 | 0.0232 | 4.82 |
| 44 | 0.0073 | 0.0886 | 12.2 |
| 45 | 0.005 | 0.0324 | 6.47 |
| 46 | 0.0051 | 0.0256 | 5 |
| 47 | 0.008 | 0.0362 | 4.5 |
| 48 | 0.0048 | 0.0268 | 6.44 |
| 49 | 0.0118 | 0.0697 | 5.91 |
| 50 | 0.0052 | 0.0341 | 6.53 |
| 51 | 0.0068 | 0.0362 | 5.07 |
| 52 | 0.0034 | 0.0139 | 4.08 |
| 53 | 0.0072 | 0.0337 | 5.12 |
| 54 | 0.0057 | 0.024 | 4.23 |
| 55 | 0.0062 | 0.026 | 4.22 |
| 56 | 0.0048 | 0.0201 | 4.21 |
| 57 | 0.0048 | 0.0208 | 4.32 |
| 58 | 0.005 | 0.0396 | 7.95 |
| 59 | 0.0064 | 0.0331 | 5.15 |
| 60 | 0.0075 | 0.0367 | 4.91 |
| 61 | 0.0143 | 0.0824 | 5.76 |
| 62 | 0.0118 | 0.0892 | 7.56 |
| 63 | 0.0079 | 0.046 | 5.84 |
| 64 | 0.0156 | 0.0745 | 4.78 |
| 65 | 0.0028 | 0.0414 | 15 |
| 66 | 0.006 | 0.033 | 5.46 |
| 67 | 0.0113 | 0.0562 | 5.2 |
| 68 | 0.0096 | 0.0556 | 5.82 |
| 69 | 0.0047 | 0.043 | 9.23 |
| 70 | 0.0076 | 0.0629 | 8.32 |
| 71 | 0.0092 | 0.0356 | 3.88 |
| 72 | 0.0066 | 0.0427 | 6.42 |
| 73 | 0.0057 | 0.0298 | 5.25 |
| 74 | 0.0375 | 0.0885 | 2.36 |
| 75 | 0.0037 | 0.0147 | 3.93 |
| 76 | 0.0165 | 0.0871 | 5.28 |
| 77 | 0.0084 | 0.0487 | 5.1 |
| 78 | 0.0050 | 0.0299 | 5.98 |
| 79 | 0.0122 | 0.0592 | 4.85 |
| 80 | 0.0094 | 0.0534 | 5.66 |
| 81 | 0.0070 | 0.0395 | 5.6429 |
| 82 | 0.0095 | 0.0572 | 6.45 |
| 83 | 0.0125 | 0.0958 | 8.1 |
| 84 | 0.0073 | 0.0353 | 4.83 |
| 85 | 0.0061 | 0.051 | 8.42 |
| 86 | 0.0048 | 0.0249 | 5.21 |
| 87 | 0.0073 | 0.0394 | 5.43 |
| 88 | 0.0063 | 0.0296 | 4.67 |
| 89 | 0.0069 | 0.0358 | 5.17 |
| 90 | 0.0111 | 0.0657 | 5.92 |
| 91 | 0.0084 | 0.0498 | 5.96 |
| 92 | 0.0085 | 0.0974 | 11.7 |
| 93 | 0.0053 | 0.0182 | 3.45 |
| 94 | 0.0032 | 0.0107 | 3.31 |
| 95 | 0.0048 | 0.0196 | 4.11 |
| 96 | 0.0056 | 0.0291 | 5.15 |
| 97 | 0.0033 | 0.0158 | 4.68 |
| 98 | 0.0087 | 0.0401 | 4.6 |
| 99 | 0.0105 | 0.0763 | 7.27 |
| 100 | 0.0156 | 0.066 | 4.23 |
| 101 | 0.0166 | 0.0806 | 4.86 |
| 102 | 0.0075 | 0.0424 | 6.03 |
| 103 | 0.0060 | 0.0399 | 6.66 |
| 104 | 0.0166 | 0.0893 | 5.38 |
| 105 | 0.0054 | 0.0285 | 5.28 |
| 106 | 0.0107 | 0.0485 | 4.53 |
| 107 | 0.0034 | 0.0166 | 4.47 |
| 108 | 0.0066 | 0.0383 | 5.85 |
| 109 | 0.0048 | 0.022 | 4.38 |
| 110 | 0.0102 | 0.0512 | 5.02 |
| 111 | 0.0048 | 0.0392 | 8.18 |
| 112 | 0.0124 | 0.0707 | 5.7 |
| 113 | 0.0063 | 0.0278 | 4.38 |
| 114 | 0.0099 | 0.0602 | 6.09 |
| 115 | 0.0072 | 0.0458 | 6.05 |
| 116 | 0.0111 | 0.0583 | 5.25 |
| 117 | 0.0081 | 0.033 | 4.09 |
| 118 | 0.0107 | 0.0814 | 7.61 |
| 119 | 0.0284 | 0.073 | 2.57 |
| 120 | 0.0032 | 0.0186 | 5.83 |
| 121 | 0.0076 | 0.0397 | 5.25 |
| 122 | 0.0167 | 0.0662 | 3.96 |
| 123 | 0.0038 | 0.0185 | 4.82 |
| 124 | 0.0041 | 0.0231 | 5.62 |
| 125 | 0.0036 | 0.0178 | 4.94 |
| 126 | 0.0048 | 0.0303 | 6.34 |
| 127 | 0.0077 | 0.0277 | 3.58 |
| 128 | 0.0103 | 0.0613 | 5.95 |
| 129 | 0.0140 | 0.0632 | 4.5143 |
| 130 | 0.0055 | 0.0258 | 4.69 |
| 131 | 0.0361 | 0.244 | 6.759 |
| 132 | 0.0660 | 0.48 | 7.2727 |
| 133 | 0.0100 | 0.0556 | 5.59 |
| 134 | 0.0058 | 0.0265 | 4.6 |
| 135 | 0.0044 | 0.0339 | 7.72 |
| 136 | 0.0037 | 0.0118 | 3.22 |
| 137 | 0.0148 | 0.0487 | 3.2905 |
| 138 | 0.0360 | 0.195 | 5.4167 |
| 139 | 0.0210 | 0.115 | 5.4762 |
| 140 | 0.0055 | 0.034 | 6.2044 |
| 141 | 0.0085 | 0.019 | 2.2379 |
| 142 | 0.1560 | 1.4 | 8.9744 |
| 143 | 0.0480 | 0.605 | 12.604 |
| 144 | 0.0245 | 0.307 | 12.531 |
| 145 | 0.0295 | 0.154 | 5.2203 |
| 146 | 0.0060 | 0.0349 | 5.8167 |
| 147 | 0.0304 | 0.211 | 6.9408 |
| 148 | 0.0070 | 0.0345 | 4.9286 |
| 149 | 0.0239 | 0.261 | 10.921 |
| 150 | 0.0110 | 0.0579 | 5.2636 |
| 151 | 0.0105 | 0.0635 | 6.0476 |
| 152 | 0.0105 | 0.037 | 3.5238 |
| 153 | 0.0095 | 0.051 | 5.3741 |
| 154 | 0.0670 | 0.265 | 3.9552 |
| 155 | 0.0105 | 0.0849 | 8.0857 |
| 156 | 0.0154 | 0.0811 | 5.2662 |
| 157 | 0.0171 | 0.0955 | 5.5848 |
| 158 | 0.0068 | 0.0546 | 8.0412 |
| 159 | 0.0079 | 0.0392 | 4.9873 |
| 160 | 0.0113 | 0.043 | 3.8053 |
| 161 | 0.0092 | 0.0427 | 4.6212 |
| 162 | 0.0063 | 0.0319 | 5.0958 |
| 163 | 0.0100 | 0.0597 | 5.97 |
| 164 | 0.0054 | 0.0497 | 9.1529 |
| 165 | 0.0092 | 0.0828 | 9.0393 |
| 166 | 0.0045 | 0.0298 | 6.5639 |
| 167 | 0.0078 | 0.0669 | 8.5769 |
| 168 | 0.0075 | 0.0558 | 7.4599 |
| 169 | 0.0049 | 0.0248 | 5.082 |
| 170 | 0.0047 | 0.0306 | 6.4831 |
| 171 | 0.0128 | 0.0603 | 4.7109 |
| 172 | 0.0061 | 0.0284 | 4.6557 |
| 173 | 0.0084 | 0.0465 | 5.5095 |
| 174 | 0.0044 | 0.0248 | 5.6751 |
| 175 | 0.0041 | 0.0261 | 6.335 |
| 176 | 0.0093 | 0.0596 | 6.4155 |
| 177 | 0.0052 | 0.0581 | 11.238 |
| 178 | 0.0109 | N.D | N.D |
| 179 | 0.0117 | 0.113 | 9.658 |
| 180 | 0.0078 | N.D | N.D |
| 181 | 0.0194 | N.D | N.D |
| 182 | 0.0097 | N.D | N.D |
| 183 | 0.0143 | N.D | N.D |
| 184 | 0.0061 | N.D | N.D |
| 185 | 0.0051 | N.D | N.D |
| 186 | 0.0047 | N.D | N.D |
| 187 | 0.0069 | N.D | N.D |
| 188 | 0.0113 | N.D | N.D |
| 189 | 0.0299 | N.D | N.D |
| 190 | 0.0077 | N.D | N.D |
| 191 | 0.0131 | N.D | N.D |
| 192 | 0.0083 | N.D | N.D |
| 193 | 0.0062 | N.D | N.D |
| 194 | 0.0026 | N.D | N.D |
| 195 | 0.0041 | N.D | N.D |
| 196 | 0.0089 | N.D | N.D |
| 197 | 0.0023 | N.D | N.D |
| 198 | 0.0349 | N.D | N.D |
| 199 | 0.0244 | N.D | N.D |
| 200 | 0.0155 | N.D | N.D |
| 201 | 0.0394 | N.D | N.D |
| 202 | 0.0256 | N.D | N.D |
| 203 | 0.0091 | N.D | N.D |
| 204 | 0.0111 | N.D | N.D |
| 205 | 0.0126 | N.D | N.D |
| 206 | 0.0470 | N.D | N.D |
| 207 | 0.0230 | N.D | N.D |
| 208 | 0.0301 | N.D | N.D |
| 209 | 0.0486 | N.D | N.D |
| 210 | 0.0395 | N.D | N.D |
| 211 | 0.0157 | N.D | N.D |
| 212 | 0.0060 | N.D | N.D |
| 213 | 0.0053 | N.D | N.D |
| 214 | 0.0035 | N.D | N.D |
| 215 | 0.0147 | N.D | N.D |
| 216 | 0.0449 | N.D | N.D |
| 217 | 0.0453 | N.D | N.D |
| 218 | 0.0142 | N.D | N.D |
| 219 | 0.1312 | N.D | N.D |
| 220 | 0.0907 | N.D | N.D |
| 221 | 0.1212 | N.D | N.D |
| 222 | 0.0550 | N.D | N.D |
| 223 | N.D | N.D | N.D |
| 224 | N.D | N.D | N.D |
| 225 | N.D | N.D | N.D |
| 226 | 0.0365 | N.D | N.D |
| 227 | 0.0254 | N.D | N.D |
| 228 | 0.0146 | 0.1610 | 11.0 |
| 229 | 0.005018 | 0.0222 | 4.4 |
| 230 | 0.351 | 0.964 | 2.7 |
| 231 | 0.03914 | | |
| 232 | 0.0328 | 0.0657 | 2.0 |
| 233 | 0.006936 | | |
| 234 | 0.00221 | | |
| 235 | 0.0208 | 0.0968 | 4.7 |
| 236 | 0.004184 | | |
| 237 | 0.007 | | |
| 238 | 0.009069 | | |
| 239 | 0.01278 | | |
| 240 | 0.04457 | | |
| 241 | 0.05863 | | |

N.D: Not determined

### Example C: In Vitro Cytotoxicity of the Bcl-2 Inhibitors in Modified Cells Expressing G101V, D103Y, D103V, D103E, F104I, F104L, D111A, A113G, R129L V156D Mutants

KMS-12-PE (ACC 606) were purchased from the Leibniz-Institute DSMZ (Braunschweig, Germany) and were grown at 37° C. in a humidified atmosphere with 5% CO₂ in media recommended by the suppliers. Lentiviral particles containing Bcl-2 wild type (also named “Bcl2 WT”), Bcl-2 mutated on G101V (also named “Bcl2 G101V”), Bcl-2 mutated on D103Y (also named “Bcl2 D103Y”), Bcl-2 mutated on D103V (also named “Bcl2 D103V”), Bcl-2 mutated on D103E (also named “Bcl2 D103E”), Bcl-2 mutated on F₁₀₄I (also named “Bcl2 F104I”), Bcl-2 mutated on F104L (also named “Bcl2 F104L”), Bcl-2 mutated on D111A (also named “Bcl2 D111A”), Bcl-2 mutated on A113G (also named “Bcl2 A113G”), Bcl-2 mutated on R₁₂₉L (also named “Bcl2 R₁₂₉L”), Bcl-2 mutated on V156D (also named “Bcl2 V156D”) were cloned into pcLV-CMV-DEST-IRES-TagRFP. Lentiviral particles (1×10⁶) were mixed with Polybrene at 8 μg/ml and transduced by spinoculation for 1 h at 32° C. and incubated overnight. After 8 days, TagRFP positive-cells were sorted by FACS. BCL2 expression was monitored by immunoblotting using anti-Flag and anti-BCL2 antibodies. Cells were seeded into 96-wells plates and treated with 1:3.16 serial dilution of compounds (9 different concentrations). Cell viability was assessed using CellTiterGlo reagent following treatment for 72 h. Results were normalized to the viability of cells without compounds (control wells). The C50 values were calculated using nonlinear regression algorithms in XCell software.

The results are summarised in Table 3. They show that the compounds of the invention inhibited cell viability in cell lines overexpressing Bcl-2 wild-type and a panel of Bcl-2 mutants. The compounds of the invention demonstrated an overall good cytotoxic profile among the different Bcl-2 mutants reported from the clinical data observed post-venetoclax treatment.

TABLE 3

| | | Example | Example | Example | Example |
|---|---|---|---|---|---|
| | ABT199 | 21 | 27 | 43 | 45 |
| Cell line | C₅₀ nM | C₅₀ nM | C₅₀ nM | C₅₀ nM | C₅₀ nM |
| KMS12PE | 5.5 | 0.24 | 0.19 | 0.22 | 0.32 |
| KMS12PE | 30.2 | 2.56 | 1.53 | 1.80 | 3.3 |
| Bcl2 WT | | | | | |
| KMS12PE | 1049.2 | 9.36 | 7.19 | 10.04 | 18.17 |
| Bcl2 G101V | | | | | |
| KMS12PE | 1139.6 | 2.10 | 1.1 | 1.28 | 0.2 |
| Bcl2 D103Y | | | | | |
| KMS12PE | 1301.8 | 44.99 | 29.14 | 29.26 | 114.05 |
| Bcl2 F104I | | | | | |
| KMS12PE | 22.4 | 1.27 | 0.86 | 1.13 | 2.05 |
| Bcl2 D111A | | | | | |
| KMS12PE | 260.4 | 2.36 | 1.44 | 1.44 | 2.7 |
| Bcl2 D103E | | | | | |
| KMS12PE | 120.1 | 4.06 | 2.79 | 2.50 | 8.03 |
| Bcl2 A113G | | | | | |
| KMS12PE | 42.6 | 1.51 | 0.99 | 0.99 | 1.89 |
| Bcl2 R129L | | | | | |
| KMS12PE | 139.4 | 9.77 | 3.84 | 6.63 | 0.96 |
| Bcl2 V156D | | | | | |
| KMS12PE | 589.5 | 3.34 | 1.55 | 2.00 | 2.7 |
| Bcl2 D103V | | | | | |
| KMS12PE | 1418.8 | 15.76 | 5.51 | 8.49 | 22.46 |
| Bcl2 F104L | | | | | |
| | Example | Example | Example | Example | Example |
| | 51 | 52 | 53 | 55 | 66 |
| Cell line | C₅₀ nM | C₅₀ nM | C₅₀ nM | C₅₀ nM | C₅₀ nM |
| KMS12PE | 0.34 | 0.21 | 0.35 | 0.33 | 0.2 |
| KMS12PE | 2.82 | 2.07 | 2.88 | 2.61 | 1.74 |
| Bcl2 WT | | | | | |
| KMS12PE | 20.22 | 10.59 | 16.34 | 13.65 | 10.14 |
| Bcl2 G101V | | | | | |
| KMS12PE | 2.79 | 1.8 | 2.61 | 2.5 | 1.3 |
| Bcl2 D103Y | | | | | |
| KMS12PE | 320.53 | 11.1 | 72.70 | 57.78 | 65.18 |
| Bcl2 F104I | | | | | |
| KMS12PE | 3.21 | 1.21 | 2.19 | 2.2 | 1.57 |
| Bcl2 D111A | | | | | |
| KMS12PE | 2.81 | 1.86 | 2.76 | 2.91 | 1.89 |
| Bcl2 D103E | | | | | |
| KMS12PE | 9.24 | 3.99 | 5.68 | 5.53 | 2.89 |
| Bcl2 A113G | | | | | |
| KMS12PE | 3.22 | 1.89 | 2.38 | 2.46 | 1.32 |
| Bcl2 R129L | | | | | |
| KMS12PE | 31.85 | 10.63 | 21.45 | 14.89 | 7.65 |
| Bcl2 V156D | | | | | |
| KMS12PE | 4.63 | 2.78 | 5.38 | 4.48 | 2.22 |
| Bcl2 D103V | | | | | |
| KMS12PE | 58.89 | 11.1 | 25.51 | 19.73 | 24.14 |
| Bcl2 F104L | | | | | |

| | Example 77 | Example 78 | Example 107 |
|---|---|---|---|
| Cell line | C₅₀ nM | C₅₀ nM | C₅₀ nM |
| KMS12PE | 0.42 | 0.12 | 0.15 |
| KMS12PE | 4.75 | 1.41 | 1.59 |
| Bcl2 WT | | | |
| KMS12PE | 28.25 | 8.35 | 9.13 |
| Bcl2 G101V | | | |
| KMS12PE | 3.31 | 1 | 0.97 |
| Bcl2 D103Y | | | |
| KMS12PE | 156.17 | 21.84 | 23.58 |
| Bcl2 F104I | | | |
| KMS12PE | 3.64 | 1.14 | 1.36 |
| Bcl2 D111A | | | |
| KMS12PE | 4.69 | 1.18 | 1.22 |
| Bcl2 D103E | | | |
| KMS12PE | 7.86 | 2.11 | 2.82 |
| Bcl2 A113G | | | |
| KMS12PE | 2.81 | 0.9 | 1.12 |
| Bcl2 R129L | | | |
| KMS12PE | 19.06 | 5.88 | 5.93 |
| Bcl2 V156D | | | |
| KMS12PE | 6.28 | 1.59 | 1.94 |
| Bcl2 D103V | | | |
| KMS12PE | 31.91 | 8.71 | 7.04 |
| Bcl2 F104L | | | |

### Example D: Pharmacodynamics and Tumor Regression Study

The in vivo therapeutic and pharmacodynamic effects of Bcl-2-targeting small molecules were determined using the following models upon intravenous (IV) administration:
(i) RS4; 11 B-cell Acute Lymphoblastic Leukemia xenograft,
(ii) KMS-12-PE BCL2 WT multiple myeloma xenograft,
(iii) KMS-12-PE BCL2 G101V multiple myeloma xenograft.

### Materials and Methods

The anti-tumor activity of some exemplary compounds of the invention was evaluated in the previous models as follows: cells were subcutaneously grafted into immunosuppressed mice. When the tumor mass had reached an average volume of about 150 mm³, mice were treated with the various compounds (IV). Tumor size was measured three times per week using electronic calipers.

At the end of the treatment cycle, tumor growth inhibition was calculated using the formula:

$$( 1 - \frac{Median ( DTV at Dx in treated group )}{Median ( DTV at Dx in Control group )} ) \times 100$$

DTV (Delta Tumor Volume) at Dx was calculated as: TV at Dx−TV at Randomization.

The compounds of the invention display significant anti-tumor activity during the treatment period.

The ability of some exemplary compounds of the invention to induce apoptosis was also evaluated in vivo using the previous xenograft model as follows: cells were subcutaneously grafted into immunosuppressed mice. When tumors had reached the appropriate volume, mice (3 per group) were treated once with the various compounds (IV). Tumor samples were recovered several hours after dosing and lysed. Cleared lysates were used for cleaved PARP and cleaved caspase 3 quantification (as markers of apoptosis induction) using the Meso Scale Discovery (MSD) Elisa platform test. Results are expressed as the ratio between cleaved PARP or cleaved caspase 3 in treated mice over control mice.

The compounds of the invention efficiently induce apoptosis in the tumors in vivo.

### Example E: Platelet Depletion in SCID Mice

Pharmacokinetic (PK) and platelet toxicity were investigated in SCID mice. Compounds were administered at 25 mg/kg by IV bolus, and formulated in PEG/Ethanol/NaCl 0.9% (40/10/50 v/v/v).

Blood samples (3 animals/time point and 3 time points/animal) were collected over 24 h after dosing: 0.05 h, 0.5 h, 1 h, 3 h, 6 h and 24 h for PK and 6 h and 24 h for platelets.

PK samples were analyzed by liquid chromatography using tandem mass spectrometry detection (LC-MS/MS) and platelet count (109/L) was measured using an hematology analyzer. Percentage of platelet loss was calculated at both time points (T+6 h and 24 h) as compared to a control group of 6 animals. PK parameters (Cinf and AUC₂₄, which correspond to the concentration at the end of bolus administration and the area under curve over 24 h, respectively) were also determined using a non-compartmental analysis.

Platelet count and the associated PK parameters following the administration of exemplary Bcl-2 inhibitors were determined. The results are presented in Table 4.

TABLE 4

PK parameters

| | | | |
|---|---|---|---|
| Example | Platelet loss (%) | Cinf | AUC₂₄ |

| No | 6 h | 24 h | (μM) | (μM · h) |
|---|---|---|---|---|
| 14 | 16.1 | 12.5 | 13.5 | 18.5 |
| 21 | 58.4 | 30.3 | 21.0 | 12.0 |
| 23 | 19.6 | 12.8 | 6.8 | 10.8 |
| 27 | 18.6 | 11.0 | 12.2 | 19.5 |
| 28 | 23.2 | 15.2 | 18.2 | 38.3 |
| 33 | 33.5 | 22.5 | 8.4 | 12.6 |
| 34 | 33.7 | 18.2 | 8.2 | 9.7 |
| 39 | 26.9 | 20.2 | 26.5 | 26.7 |
| 43 | 25.3 | 9.2 | 15.4 | 16.9 |
| 45 | 22.0 | 27.6 | 40.8 | 34.2 |
| 52 | 34.3 | 33.0 | 18.0 | 33.7 |
| 54 | 28.6 | 22.5 | 11.4 | 9.2 |
| 55 | 26.9 | 33.9 | 17.3 | 11.4 |
| 56 | 43.5 | 26.9 | 48.5 | 30.5 |

All animals were highly exposed to the tested compounds. The Cinf and AUC₂₄ ranged between 6.8 μM to 48 μM, and 6 μM·h to 38 μM·h, respectively. In these conditions, we observed that the mean percentage of platelet loss was limited and lower than 30% whatever the time points (6 and 24 h after dosing), and ranged between 16% and 58% at T+6 h and 9% to 34% at T+24 h. These data corroborate the range of Bcl-xL activity observed for the tested compounds in the TR-FRET assay.

### Example F: CYP3A4 Inhibition Determination

The inhibitory potential was determined on CYP3 Å4 enzyme using hepatic human microsomes. A range of concentrations (adapted based on the solubility) of the tested compound were incubated in presence of a substrate having a specific metabolite of CYP3 Å4 enzyme.

Then, the formation of the metabolite was quantified using LC/MS-MS analysis and a curve corresponding to the concentration of the metabolite in function of the concentration of the test compound allowed to the calculation of the IC₅₀ value.

The IC₅₀ values were determined for exemplary Bcl-2 inhibitors. The results are presented in Table 5.

TABLE 5

| Example | IC₅₀ CYP3A4 |
|---|---|
| No | (μM) |
| 21 | 6.2 |
| 25 | 12 |
| 67 | >5 |
| 77 | >5 |
| 78 | >5 |
| 91 | 18 |
| 101 | >5 |
| 109 | >5 |
| 110 | >5 |

The compounds exhibited an IC₅₀ higher than 5 μM and up to 18 μM indicating a limited inhibitory potential towards CYP3 Å4 that allows to mitigate the CYP3 Å4 drug-drug interaction risk in clinical studies.

1-40. (canceled)

41. A compound of formula (I): 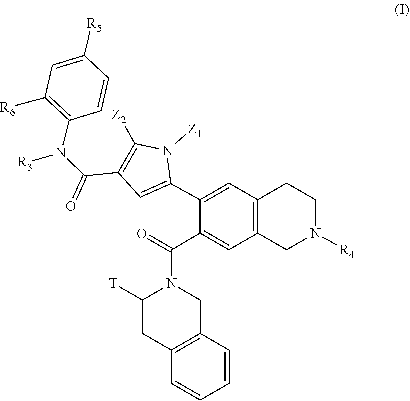
wherein:
Z₁ and Z₂ both represent a methyl group or together with the atoms carrying them form a fused piperidine group,
T represents a hydrogen atom, a linear or branched (C₁-C₆)alkyl group optionally substituted by one to three halogen atoms, a (C₁-C₄)alkylene-NR₁R₂ group, or a (C₁-C₄)alkylene-ORᵢ group,
R₁ and R₂ independently of one another represent a hydrogen atom or a linear or branched (C₁-C₆)alkyl group,
or R₁ and R₂, together with the nitrogen atom carrying them, form a heterocycloalkyl group, which heterocycloalkyl group is optionally substituted by one to three groups selected from (C₁-C₆)alkyl and halogen,
R₃ represents a group selected from: 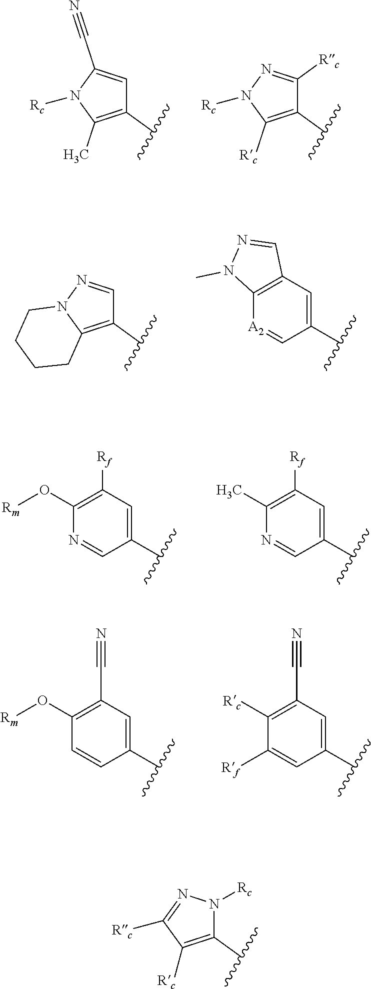
R₄ represents a group selected from: 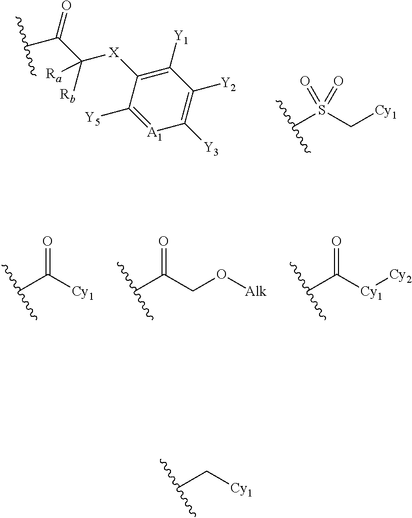
R₅ represents a hydrogen atom, a halogen atom or a hydroxy group,
R₆ represents a hydrogen, a linear or branched (C₁-C₆)alkyl group, or a halogen atom,
Alk represents a linear or branched (C₁-C₆)alkyl group,
A₁ represents C—Y₄ or a nitrogen atom,
A₂ represents C—H or a nitrogen atom,
Cy₁ represents a phenyl, a heteroaryl, a cycloalkyl or a heterocycloalkyl group, wherein the phenyl, the heteroaryl, the cycloalkyl and the heterocycloalkyl groups may be optionally substituted by one to three groups selected from linear or branched (C₁-C₆)alkyl optionally substituted by 1 to 3 halogen atoms, hydroxy, cycloalkyl, and halogen and the heterocycloalkyl group may be optionally further substituted by an oxo group,
Cy₂ represents a phenyl group or a heteroaryl group, wherein the phenyl and the heteroaryl groups may be optionally substituted by one to three groups selected from linear or branched (C₁-C₆)alkyl optionally substituted by 1 to 3 halogen atoms, hydroxy, and halogen,
X represents a bond, —O—, —S— or NRₖ,
Y₁ and Y₅ independently of one another represent a group selected from: a hydrogen atom, a halogen atom, a cyano group, a linear or branched (C₁-C₆)alkyl group, and a linear or branched (C₁-C₆)alkoxy group,
Y₂ and Y₄ independently of one another represent a group selected from a hydrogen atom, a halogen atom, a linear or branched (C₁-C₆)alkyl group, a linear or branched (C₁-C₆)alkoxy group, and a heterocycloalkyl group optionally substituted by linear or branched (C₁-C₆)alkyl,
Y₃ represents a group selected from a hydrogen atom, a halogen atom, a linear or branched (C₁-C₆)alkyl group, a linear or branched (C₁-C₆)alkynyl group, —(C₁-C₄)alkylene-OR₁, a linear or branched (C₁-C₆)alkoxy group, —O-phenyl, —S-phenyl, —O—(C₁-C₄)alkylene-Cy₃, —O—(C₁-C₄)alkylene-Cy₄, —O-Cy₃, —O—(C₁-C₄)alkylene-NR_{g}Rₕ, —(C₁-C₄)alkylene-Cy₃, —(C₁-C₄)alkylene-Cy₄, Cy₃, Cy₄, and: 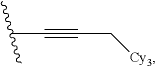
wherein the alkylene moiety of the preceding groups may be linear or branched,
Cy₃ represents a heterocycloalkyl group optionally substituted by one to three groups selected from linear or branched (C₁-C₆)alkyl optionally substituted by 1 to 3 halogen atoms, hydroxy, cycloalkyl, heterocycloalkyl, and halogen,
Cy₄ represents a cycloalkyl group optionally substituted by one to three groups selected from linear or branched (C₁-C₆)alkyl optionally substituted by 1 to 3 halogen atoms, hydroxy, cycloalkyl, heterocycloalkyl, and halogen,
Rₐ and R_{b} independently of one another represent a hydrogen atom or a halogen atom,
R_{c} represents a group selected from hydrogen, a linear or branched (C₁-C₆)alkyl group optionally substituted by 1 to 3 halogen atoms, (C₁-C₆)alkylene-NR_{d}Rₑ, (C₁-C₆)alkylene-ORⱼ, cycloalkyl, heterocycloalkyl, and (C₁-C₆)alkylene-heterocycloalkyl,
R′_{c} and R″ₑ independently of one another represent a hydrogen atom or a linear or branched (C₁-C₆)alkyl group,
R_{d} and Rₑ independently of one another represent a hydrogen atom, a linear or branched (C₁-C₆)alkyl group, a cycloalkyl group or a heterocycloalkyl group,
R_{f} represents a hydrogen atom, a halogen atom or a cyano group,
R′_{f} represents a hydrogen atom or a halogen atom,
R_{g} and Rₕ independently of one another represent a hydrogen atom, a linear or branched (C₁-C₆)alkyl group optionally substituted by one to three halogen atoms, a cycloalkyl group, a heterocycloalkyl group, or a—(C₁-C₆)alkylene-heterocycloalkyl,
Rᵢ, Rⱼ, and Rₖ independently of one another represent a hydrogen atom, a linear or branched (C₁-C₆)alkyl group, or a—(C₁-C₆)alkylene-cycloalkyl group,
Rₗ represents a hydrogen atom, a linear or branched (C₁-C₆)alkyl group or a linear or branched (C₁-C₆)alkylene-heterocycloalkyl group,
Rₘ represents a hydrogen or a linear or branched (C₁-C₆)alkyl group,
its enantiomers and diastereoisomers, and addition salts thereof with a pharmaceutically acceptable acid or base.

42. The compound according to claim 41, which is a compound of formula (I-a): 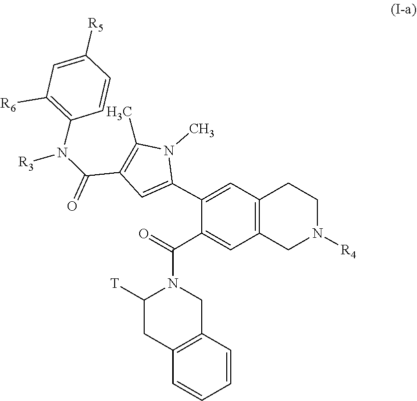
wherein R₃, R₄, R₅, R₆ and T are as defined in claim 41.

43. The compound according to claim 41, wherein R₃ 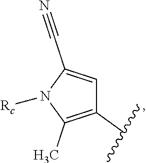
wherein R_{c} represents a group selected from: hydrogen, linear or branched (C₁-C₆)alkyl group optionally substituted by 1 to 3 halogen atoms, (C₁-C₆)alkylene-NR_{d}Rₑ, (C₁-C₆)alkylene-ORⱼ, cycloalkyl, heterocycloalkyl, and (C₁-C₆)alkylene-heterocycloalkyl.

44. The compound according to claim 43, wherein R_{c} represents a methyl group.

45. The compound according to claim 41, wherein R₄ represents 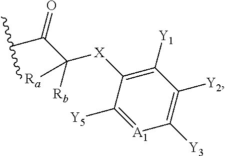
wherein Rₐ, R_{b}, X, A₁, Y₁, Y₂, Y₃, Y₅ are as defined in claim 41.

46. The compound according to claim 41, which is a compound of formula (I-b): 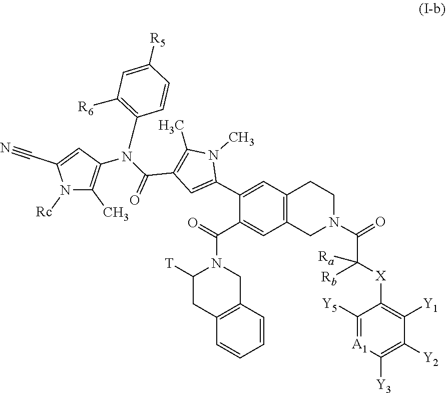
wherein R₅, R₆, Rₐ, R_{b}, R_{c}, X, A₁, Y₁, Y₂, Y₃, Y₅ and T are as defined in claim 41.

47. The compound according to claim 46, wherein R_{c} represents a methyl group.

48. The compound according to claim 41, which is a compound of formula (I-c): 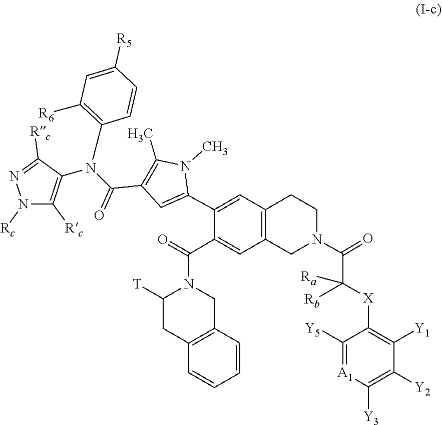
wherein R₅, R₆, Rₐ, R_{b}, Rₑ, R′_{c}, R″_{c}, X, A₁, Y₁, Y₂, Y₃, Y₅ and T are as defined in claim 41.

49. The compound according to claim 41, which is a compound of formula (I-d): 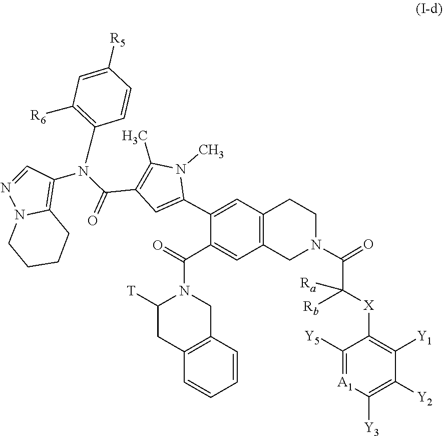
wherein R₅, R₆, Rₐ, R_{b}, X, A₁, Y₁, Y₂, Y₃, Y₅ and T are as defined in claim 41.

50. The compound according to claim 41, which is a compound of formula (I-e): 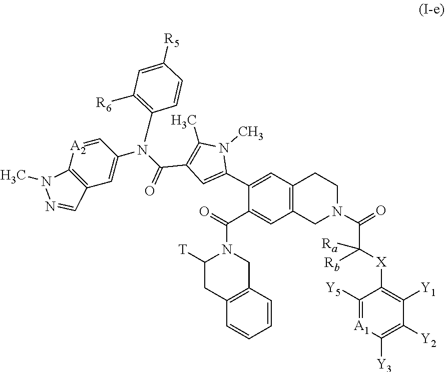
wherein R₅, R₆, Rₐ, R_{b}, X, A₁, A₂, Y₁, Y₂, Y₃, Y₅ and T are as defined in claim 41.

51. The compound according to claim 41, which is a compound of formula (I-f): 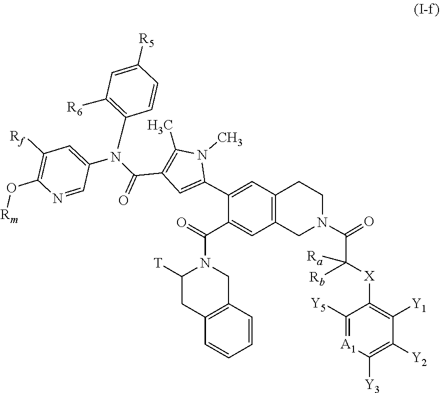
wherein R₅, R₆, Rₐ, R_{b}, R_{f}, Rₘ, X, A₁, Y₁, Y₂, Y₃, Y₅ and T are as defined in claim 41.

52. The compound according to claim 41, which is a compound of formula (I-g): 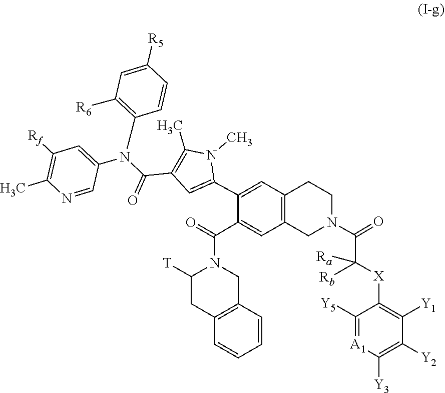
wherein R₅, R₆, Rₐ, R_{b}, R_{f}, X, A₁, Y₁, Y₂, Y₃, Y₅ and T are as defined in claim 41.

53. The compound according to claim 41, which is a compound of formula (I-h): 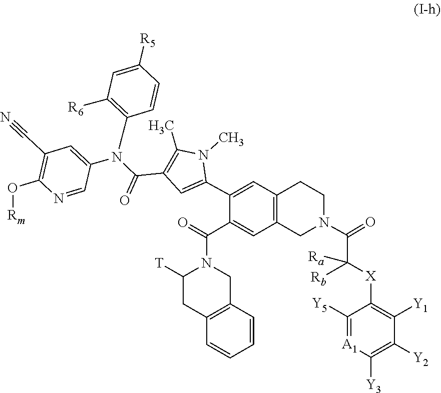
wherein R₅, R₆, Rₐ, R_{b}, Rₘ, X, A₁, Y₁, Y₂, Y₃, Y₅ and T are as defined in claim 41.

54. The compound according to claim 41, which is a compound of formula (I-i): 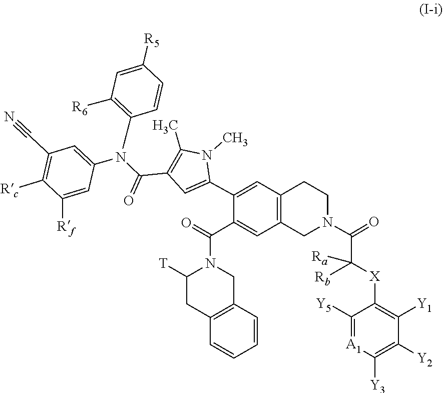
wherein R₅, R₆, Rₐ, R_{b}, R′_{c}, R′_{f}, X, A₁, Y₁, Y₂, Y₃, Y₅ and T are as defined in claim 41.

55. The compound according to claim 41, which is a compound of formula (I-j): 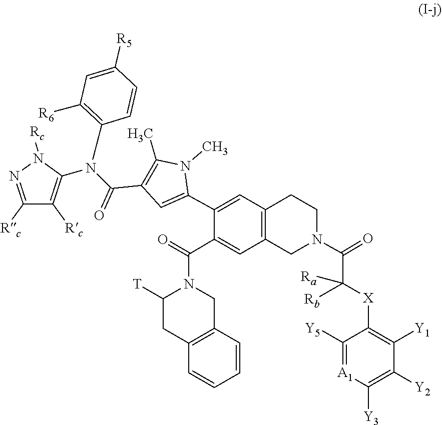
wherein R₅, R₆, Rₐ, R_{b}, R_{c}, R′_{c}, R″, X, A₁, Y₁, Y₂, Y₃, Y₅ and T are as defined in claim 41.

56. The compound according to claim 45, wherein X represents a bond.

57. The compound according to claim 45, wherein A₁ represents C—Y₄.

58. The compound according to claim 45, wherein Rₐ and R_{b} both represent a hydrogen atom.

59. The compound according to claim 45, wherein R₅ represents a hydrogen atom, a hydroxy group or a fluorine atom.

60. The compound according to claim 45, wherein R₆ represents a hydrogen atom or a fluorine atom.

61. The compound according to claim 45, wherein R₅ represents a hydroxy group and R₆ represents a hydrogen atom.

62. The compound according to claim 45, wherein A₁ represents C—H and Y₂ represents a hydrogen atom.

63. The compound according to claim 45, wherein Y₁ and Y₅ both represent a hydrogen atom, or Y₁ and Y₅ represent a fluoro atom and a hydrogen atom, respectively.

64. The compound according to claim 45, wherein Y₃ represents an —O—(C₁-C₄)alkylene-Cy₃ group.

65. The compound according to claim 64, wherein Y₃ represents a group selected from 2-(morpholin-4-yl)ethoxy, 2-(oxan-4-yl)ethoxy, 2-(4-hydroxypiperidin-1-yl)ethoxy, 2-(4-cyclopropylpiperazin-1-yl)ethoxy, 2-[4-(2,2,2-trifluoroethyl)piperazin-1-yl]ethoxy, 2-[(9aS)-octahydropyrazino[2,1-c][1,4]oxazin-8-yl]ethoxy, 2-{2-[4-(2-{1,1-dioxo-1λ⁶-thia-6-azaspiro[3.3]heptan-6-yl}ethoxy, 2-[2,6-dimethylmorpholin-4-yl]ethoxy, 2-[4-(2,2-difluoroethyl)piperazin-1-yl]ethoxy, 2-(3-fluoroazetidin-1-yl)ethoxy, 2-(3,3-difluoropyrrolidin-1-yl)ethoxy, 2-(4-fluoropiperidin-1-yl)ethoxy, 2-(thiomorpholin-4-yl)ethoxy, 2-(2-methylmorpholin-4-yl)ethoxy, 2-{6-oxa-9-azaspiro[4.5]decan-9-yl}ethoxy, 2-{4-oxa-7-azaspiro[2.5]octan-7-yl}ethoxy, 2-[4-(2-fluoroethyl)piperazin-1-yl]ethoxy, 2-(4-methylpiperazin-1-yl)ethoxy, 2-(2,2-dimethylmorpholin-4-yl)ethoxy, 2-(morpholin-4-yl)propoxy, [2-methyl-1-(morpholin-4-yl)propan-2-yl]oxy, 2-(3,3-dimethylmorpholin-4-yl)ethoxy, 2-(3-methylmorpholin-4-yl)ethoxy, and 2-(1,4-dioxan-2-yl)ethoxy.

66. The compound according to claim 41, wherein the group: 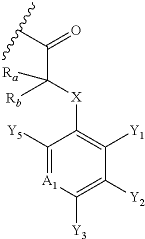
represents

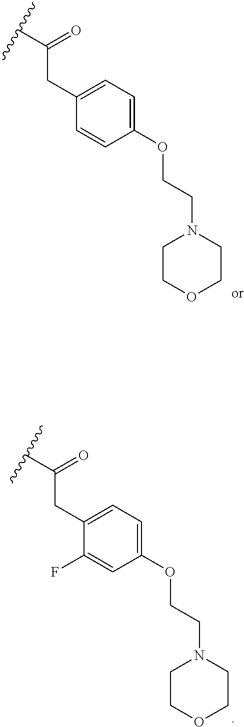 [SMILES: *C(=O)Cc1ccc(OCCN2CCOCC2)cc1 ; *C(=O)Cc1ccc(OCCN2CCOCC2)cc1F]

67. The compound according to claim 41, wherein T represents a linear or branched (C₁-C₆)alkyl group or a (C₁-C₄)alkylene-NR₁R₂ group.

68. The compound according to claim 67, wherein T represents a group selected from: methyl group, (piperidin-1-yl)methyl, (morpholin-4-yl)methyl, (piperidin-1-yl)ethyl, [(3R)-3-fluoropyrrolidin-1-yl]methyl, (4-fluoropiperidin-1-yl)methyl, [methyl(propan-2-yl)amino]methyl, (azepan-1-yl)methyl, (pyrrolidin-1-yl)methyl, [(3S)-3-methylpiperidin-1-yl]methyl, [(3R)-3-methylpiperidin-1-yl]methyl, [(1RS,5SR)-3-azabicyclo[3.1.0]hexan-3-yl]methyl, [(2S)-2-methylpiperidin-1-yl]methyl, {6-azaspiro[2.5]octan-6-yl}methyl, (4,4-difluoropiperidin-1-yl)methyl, (diethylamino)methyl, (4-methylpiperidin-1-yl)methyl, [ethyl(propan-2-yl)amino]methyl, {5-azaspiro[2.3]hexan-5-yl}methyl, (3,3-dimethylpyrrolidin-1-yl)methyl, (diisopropylamino)methyl, [ethyl(isopropyl) amino]methyl, [(3R)-3-methylpyrrolidin-1-yl]methyl, [(3S)-3-methylpyrrolidin-1-yl]methyl, [(2S)-2-methylpyrrolidin-1-yl]methyl, 5-azaspiro[2.4]heptan-5-ylmethyl, 2-azaspiro[3.3]heptan-2-ylmethyl, and aminomethyl.

69. The compound according to claim 41, which is selected from:
5-[2-benzylsulfonyl-7-[(3R)-3-methyl-3,4-dihydro-1H-isoquinoline-2-carbonyl]-3,4-dihydro-1H-isoquinolin-6-yl]-N-(5-cyano-1,2-dimethyl-pyrrol-3-yl)-N-(4-hydroxyphenyl)-1,2-dimethyl-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(morpholin-4-yl)ethoxy]phenyl}acetyl)-1,2,3,4-tetrahydroisoquinolin-6-yl}-1H-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[4-(2,2,2-trifluoroethyl)piperazin-1-yl]phenyl}acetyl)-1,2,3,4-tetrahydroisoquinolin-6-yl}-1H-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(oxan-4-yl)ethoxy]phenyl}acetyl)-1,2,3,4-tetrahydroisoquinolin-6-yl}-1H-pyrrole-3-carboxamide,
N-(5-cyano-1,2-dimethylpyrrol-3-yl)-5-[2-(2-{3-fluoro-4-[1-(2-fluoroethyl)piperidin-4-yl]phenyl}acetyl)-7-{[(3R)-3-methyl-3,4-dihydro-TH-isoquinolin-2-yl]carbonyl}-3,4-dihydro-TH-isoquinolin-6-yl]-N-(4-hydroxyphenyl)-1,2-dimethylpyrrole-3-carboxamide,
N-(5-cyano-1,2-dimethylpyrrol-3-yl)-5-{2-[2-(2-fluoro-4-methoxyphenyl)acetyl]-7-{[(3S)-3-(piperidin-1-ylmethyl)-3,4-dihydro-TH-isoquinolin-2-yl]carbonyl}-3,4-dihydro-1H-isoquinolin-6-yl}-N-(4-hydroxyphenyl)-1,2-dimethylpyrrole-3-carboxamide,
N-(5-cyano-1,2-dimethylpyrrol-3-yl)-5-[2-(2-{2-fluoro-4-[2-(oxan-4-yl)ethoxy]phenyl}acetyl)-7-{[(3S)-3-(piperidin-1-ylmethyl)-3,4-dihydro-TH-isoquinolin-2-yl]carbonyl}-3,4-dihydro-TH-isoquinolin-6-yl]-N-(4-hydroxyphenyl)-1,2-dimethylpyrrole-3-carboxamide,
N-(5-cyano-1,2-dimethyl-TH-pyrrol-3-yl)-5-{2-[2-(4-{2-[cis-2,6-dimethylmorpholin-4-yl]ethoxy}phenyl)acetyl]-7-[(3R)-3-methyl-1,2,3,4-tetrahydroisoquinoline-2-carbonyl]-1,2,3,4-tetrahydroisoquinolin-6-yl}-N-(4-hydroxyphenyl)-1,2-dimethyl-1H-pyrrole-3-carboxamide,
N-(5-cyano-1,2-dimethylpyrrol-3-yl)-5-[2-(2-{2-fluoro-4-[2-(morpholin-4-yl)ethoxy]phenyl}acetyl)-7-{[(3S)-3-(piperidin-1-ylmethyl)-3,4-dihydro-TH-isoquinolin-2-yl]carbonyl}-3,4-dihydro-1H-isoquinolin-6-yl]-N-(4-hydroxyphenyl)-1,2-dimethylpyrrole-3-carboxamide,
5-[2-(2-{4-[(9aS)-octahydropyrazino[2,1-c][1,4]oxazin-8-yl]-2-fluorophenyl}acetyl)-7-[(3R)-3-methyl-1,2,3,4-tetrahydroisoquinoline-2-carbonyl]-1,2,3,4-tetrahydroisoquinolin-6-yl]-N-(5-cyano-1,2-dimethyl-TH-pyrrol-3-yl)-N-(4-hydroxyphenyl)-1,2-dimethyl-1H-pyrrole-3-carboxamide,
N-(5-cyano-1,2-dimethylpyrrol-3-yl)-5-[2-(2-{2-fluoro-4-[2-(oxan-4-yl)ethoxy]phenyl}acetyl)-7-{[(3S)-3-(methoxymethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide,
N-(5-cyano-1,2-dimethyl-pyrrol-3-yl)-5-[2-[2-[2-fluoro-4-(2-tetrahydropyran-4-ylethoxy)phenyl]acetyl]-7-[(3S)-3-(morpholinomethyl)-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(thiomorpholin-4-yl)ethoxy]phenyl}acetyl)-1,2,3,4-tetrahydroisoquinolin-6-yl}-1H-pyrrole-3-carboxamide,
N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-((2R)-2-methylmorpholin-4-yl)ethoxy]phenyl}acetyl)-1,2,3,4-tetrahydroisoquinolin-6-yl}-1H-pyrrole-3-carboxamide.
5-{2-benzoyl-7-[(3R)-3-methyl-1,2,3,4-tetrahydroisoquinoline-2-carbonyl]-1,2,3,4-tetrahydroisoquinolin-6-yl}-N-(5-cyano-1,2-dimethyl-1H-pyrrol-3-yl)-N-(4-hydroxyphenyl)-1,2-dimethyl-1H-pyrrole-3-carboxamide,
N-(5-cyano-1,2-dimethylpyrrol-3-yl)-N-(4-hydroxyphenyl)-1,2-dimethyl-5-[2-(2-{4-[2-(morpholin-4-yl)ethoxy]phenyl}acetyl)-7-{[(3S)-3-(piperidin-1-ylmethyl)-3,4-dihydro-H-isoquinolin-2-yl]carbonyl}-3,4-dihydro-1H-isoquinolin-6-yl]pyrrole-3-carboxamide,
N-[5-cyano-1-(difluoromethyl)-2-methylpyrrol-3-yl]-N-(4-hydroxyphenyl)-1,2-dimethyl-5-[2-(2-{4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]pyrrole-3-carboxamide,
N-[5-cyano-1-(difluoromethyl)-2-methylpyrrol-3-yl]-5-[2-(2-{2-fluoro-4-[2-(morpholin-4-yl)ethoxy]phenyl}acetyl)-7-{[(3S)-3-(piperidin-1-ylmethyl)-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide,
N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[methyl(propan-2-yl)amino]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide,
N-(5-cyano-1,2-dimethylpyrrol-3-yl)-5-{2-[2-(4-{2-[cyclopropyl(methyl)amino]ethoxy}-2-fluorophenyl)acetyl]-7-{[(3S)-3-(pyrrolidin-1-ylmethyl)-3,4-dihydro-1H-isoquinolin-2-yl]carbonyl}-3,4-dihydro-1H-isoquinolin-6-yl}-N-(4-hydroxyphenyl)-1,2-dimethylpyrrole-3-carboxamide,
N-(5-cyano-1,2-dimethyl-1H-pyrrol-3-yl)-5-[2-(2-{2-fluoro-4-[2-((2R or S)-2-methylmorpholin-4-yl)ethoxy]phenyl}acetyl)-7-[(3S)-3-[(piperidin-1-yl)methyl]-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide,
N-[5-cyano-1-(difluoromethyl)-2-methylpyrrol-3-yl]-N-(4-hydroxyphenyl)-1,2-dimethyl-5-[2-(2-{2-fluoro-4-[2-(morpholin-4-yl)ethoxy]phenyl}acetyl)-7-{[(3S)-3-(pyrrolidin-1-ylmethyl)-3,4-dihydro-1H-isoquinolin-2-yl]carbonyl}-3,4-dihydro-1H-isoquinolin-6-yl]pyrrole-3-carboxamide,
N-(5-cyano-1,2-dimethylpyrrol-3-yl)-5-[2-(2-{4-[2-(4,4-difluoropiperidin-1-yl)ethyl]phenyl}acetyl)-7-{[(3R)-3-methyl-3,4-dihydro-1H-isoquinolin-2-yl]carbonyl}-3,4-dihydro-1H-isoquinolin-6-yl]-N-(4-hydroxyphenyl)-1,2-dimethylpyrrole-3-carboxamide, —N-(5-cyano-1,2-dimethyl-TH-pyrrol-3-yl)-5-[2-(2-{4-[2-(4-fluoropiperidin-1-yl)ethoxy]phenyl}acetyl)-7-[(3R)-3-methyl-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide,
N-(5-cyano-1,2-dimethylpyrrol-3-yl)-N-(4-hydroxyphenyl)-1,2-dimethyl-5-[2-(2-{4-[2-(oxan-4-yl)ethoxy]phenyl}acetyl)-7-{[(3S)-3-(piperidin-1-ylmethyl)-3,4-dihydro-TH-isoquinolin-2-yl]carbonyl}-3,4-dihydro-1H-isoquinolin-6-yl]pyrrole-3-carboxamide, and
N-(5-cyano-1,2-dimethyl-1H-pyrrol-3-yl)-5-[2-(2-{2-fluoro-4-[2-(morpholin-4-yl)ethoxy]phenyl}acetyl)-7-[(3S)-3-{[(3S)-3-methylpiperidin-1-yl]methyl}-1,2,3,4-tetrahydroisoquinoline-2-carbonyl]-1,2,3,4-tetrahydroisoquinolin-6-yl]-N-(4-hydroxyphenyl)-1,2-dimethyl-1H-pyrrole-3-carboxamide.

70. A pharmaceutical composition comprising the compound according to claim 41, or an addition salt thereof with a pharmaceutically acceptable acid or base, in combination with one or more pharmaceutically acceptable excipients.

71. A method of treating a condition requiring a pro-apoptotic agent in a subject in need thereof, comprising administration of the compound according to claim 41, alone or in combination with one or more pharmaceutically acceptable excipients.

72. A method of treating a condition selected from cancers, auto-immune diseases or immune system diseases in a subject in need thereof, comprising administration of the compound according to claim 41, alone or in combination with one or more pharmaceutically acceptable excipients.

73. The method according to claim 72, wherein the cancer is a haematological malignancy or a solid tumor.

74. The method according to claim 73, wherein the haematological malignancy is myeloma, including multiple myeloma, lymphoma, including Non-Hodgkin Lymphoma (NHL), or leukemia, including Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML).

75. The method according to claim 73, wherein the solid tumor is selected from bladder, brain, breast, uterus, esophagus and liver cancers, colorectal cancer, renal cancer, melanoma, ovarian cancer, prostate cancer, pancreatic cancer and lung cancer.

76. A method of treating a cancer selected from myeloma, including multiple myeloma, lymphoma, including Non-Hodgkin Lymphoma (NHL), or leukemia, including Chronic Lymphocytic Leukemia (CLL), T-cell Acute Lymphoblastic Leukemia (T-ALL), B-cell Acute Lymphoblastic Leukemia (B-ALL) and Acute Myelogenous Leukemia (AML), bladder, brain, breast, uterus, esophagus and liver cancers, colorectal cancer, renal cancer, melanoma, ovarian cancer, prostate cancer, pancreatic cancer and lung cancer, including non-small-cell lung cancer and small-cell lung cancer in a subject in need thereof, comprising administration of the compound according to claim 41, alone or in combination with one or more pharmaceutically acceptable excipients.

77. A combination of the compound according to claim 41, and an anti-cancer agent selected from genotoxic agents, mitotic poisons, anti-metabolites, proteasome inhibitors, kinase inhibitors and antibodies.

78. A pharmaceutical composition comprising the combination according to claim 37 in combination with one or more pharmaceutically acceptable excipients.

79. A method of treating cancer in a subject in need thereof, comprising administration of the combination according to claim 77, alone or in combination with one or more pharmaceutically acceptable excipients.

80. A method of treating cancer requiring radiotherapy in a subject in need thereof, comprising administration of the compound according to claim 41, alone or in combination with one or more pharmaceutically acceptable excipients, in combination with radiotherapy.


SEQUENCE LISTING (7 sequences)

SEQ ID NO: 1 - protein, 24 aa, Homo sapiens
QWAREIGAQLRRMADDLNAQYERR

SEQ ID NO: 2 - protein, 521 aa, Homo sapiens
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGSENLYFQCMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRHHHHHHHH

SEQ ID NO: 3 - protein, 521 aa, Homo sapiens
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGSENLYFQCMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAVDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRHHHHHHHH

SEQ ID NO: 4 - protein, 521 aa, Homo sapiens
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGSENLYFQCMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDYFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRHHHHHHHH

SEQ ID NO: 5 - protein, 521 aa, Homo sapiens
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGSENLYFQCMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDISRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRHHHHHHHH

SEQ ID NO: 6 - protein, 521 aa, Homo sapiens
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGSENLYFQCMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRAFAEMSSQLHLTPFTARGRFATVVEELFRDGVNWGRIVAFFEFGGVMCVESVNREMSPLVDNIALWMTEYLNRHLHTWIQDNGGWDAFVELYGPSMRHHHHHHHH

SEQ ID NO: 7 - protein, 509 aa, Homo sapiens
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGSENLYFQCSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGHHHHHHHH